(19) United States
(12) Patent Application Publication
(10) Pub. No.: US 2021/0380548 A1
(43) Pub. Date: Dec. 9, 2021

(54) Fused [1,2,4]Thiadiazine Derivatives Which Act as KAT Inhibitors of the MYST Family

(71) Applicant: CTxT Pty Limited, Parkville (AU)

(72) Inventors: Benjamin Joseph Morrow, Parkville (AU); Richard Charles Foitzik, Parkville (AU); Michelle Ang Camerino, Parkville (AU); H. Rachel Lagiakos, Parkville (AU); Scott Raymond Walker, Parkville (AU); Ylva Elisabet Bergman Bozikis, Parkville (AU); Graeme Irvine Stevenson, Parkville (AU); Anthony Nicholas Cuzzupe, Mount Eliza (AU); Paul Anthony Stupple, Parkville (AU)

(21) Appl. No.: 16/642,290

(22) PCT Filed: Aug. 31, 2018
(86) PCT No.: PCT/EP2018/073431

§ 371 Date: Feb. 26, 2020

(30) Foreign Application Priority Data
Aug. 31, 2017 (GB) 1713962.7

(51) Int. Cl. C07D 285/24; A61K 45/06

(52) U.S. Cl. CPC C07D 285/24; A61K 45/06

(57) ABSTRACT

A compound of formula (I):

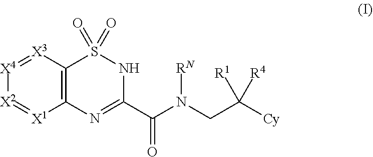

which inhibits the activity of one or more KATs of the MYST family, i.e., TIP60, KAT6B, MOZ, HBO1, and MOF.

The present invention relates to compounds which act as Lysine Acetyl Transferase (KAT) inhibitors of the MYST family.

### BACKGROUND TO THE INVENTION

The MYST family is the largest family of KATs and is named after the founding members in yeast and mammals: MOZ, Ybf2/Sas3, Sas2 and TIP60 (Dekker 2014). MYST proteins mediate many biological functions including gene regulation, DNA repair, cell-cycle regulation and development (Avvakumov 2007; Voss 2009). The KAT proteins of the MYST family play key roles in post-translational modification of histones and thus have a profound effect on chromatin structure in the eukaryotic nucleus (Avvakumov 2007). The family currently comprises five mammalian KATs: TIP60 (KAT5; HTATIP; MIM 601409), MOZ (KAT6A; MIM 601408; MYST3), MORF (KAT6b; QKF; MYST4), HBO (KAT8; HBO1; MYST2) and MOF (KAT8; MYST1) (Voss 2009). These five members of the MYST family are present in humans and malfunction of MYST proteins is known to be associated with cancer (Avvakumov 2007). The most frequently used names for members of the MYST family are:

| Common | MYST | Systematic |
|---|---|---|
| name | name | name |
| MOF | MYST1 | KAT8 |
| HBO | MYST2 | KAT7 |
| MOZ | MYST3 | KAT6A |
| MORF | MYST4 | KAT6B |
| TIP60 | | KAT5 |

MYST Functional Domains

MYST proteins function in multisubunit protein complexes including adaptors such as ING proteins that mediate DNA binding (Avvakumov 2007). For instance, TIP60 is affiliated to the NuA4 multiprotein complex (which embraces more than 16 members) (Zhang 2017). However, there have also been some reports of a helix-turn-helix DNA-binding motif within the structure of the MOZ protein itself (Holbert 2007), which suggests the capacity to bind directly to DNA.

The acetyltransferase activity of MYST proteins is effected by the MYST domain (the catalytic domain). The MYST domain contains an acetyl-coenzyme A binding motif, which is structurally conserved with other HATs, and an unusual C₂HC-type zinc finger (Voss 2009). The highly conserved MYST domain, including the acetyl-CoA binding motif and zinc finger, is considered to be the defining feature of this family of enzymes (Avvakumov 2007).

Role of MYST Proteins

Acetylation of histone residues is generally associated with transcriptional activation. However, in some instances, transcriptional repression has also been attributed to MYST proteins (Voss 2009). The individual members of the MYST family are known to participate in a broad range of important biochemical interactions:

HBO1 positively regulates initiation of DNA replication (Avvakumov 2007; Aggarwal 2004; Doyon 2006; Iizuka 2006) via acetylation of histone substrates, which presumably leads to a more accessible chromatin conformation (Avvakumov 2007, Iizuka 2006). HBO1 is also known to play a role in the pathogenesis of breast cancer by promoting an enrichment of cancer stem-like cells (Duong 2013) and by destabilising the estrogen receptor α (ERα) through ubiquinitiation, which proceeds via the histone-acetylating activity of HBO1 (Iizuka 2013). HBO1 has also been implicated in Acute myeloid leukaemia (AML) (Shi 2015).

TIP60 (KAT5) is the most studied member of the MYST family. TIP60 plays an important role not only in the regulation of transcription but also in the process of DNA damage repair, particularly in DNA double-strand breaks (DSB) (Gil 2017). TIP60 can acetylate p53, ATM and c-Myc. TIP60 and MOF specifically acetylate lysine 120 (K120) of p53 upon DNA damage (Avvakumov 2007). TIP60 has also been implicated in being important for regulatory T-cell (Treg) biology. FOXP3 is the master regulator in the development and function of Tregs and it has been shown that acetylation of FOXP3 by TIP60 is essential for FOXP3 activity (Li 2007, Xiao 2014). Underscoring this, conditional TIP60 deletion in mice leads to a scurfy-like fatal autoimmune disease, mimicking a phenotype seen in FOXP3 knock out mice (Xiao 2014). In cancer, Treg cells can facilitate tumour progression by suppressing adaptive immunity against the tumour.

MOF (“males absent on the first”) was originally identified as one of the components of the dosage compensation in Drosophila, and was classified as a member of the MYST family based on functional studies and sequence analysis (Su 2016). The human ortholog exhibits significant similarity to drosophila MOF; containing an acetyl-CoA-binding site, a chromodomain (which binds histones) and a C₂HC-type zinc finger (Su 2016). MOF is a key enzyme for acetylating histone H4K16, and MOF-containing complexes are implicated in various essential cell functions with links to cancer (Su 2016). Besides the global reduction of histone acetylation, depletion of MOF in mammalian cells can result in abnormal gene transcription, particularly causing abnormal expression of certain tumor suppressor genes or oncogenes, suggesting a critical role of MOF in tumorigenesis (Su 2016). For example, KAT activity of MOF has been shown to be required to sustain MLL-AF9 leukemia and may be important for multiple AML subtypes (Valerio 2017).

KAT6B (Querkopf) was first identified in a mutation screen for genes regulating the balance between proliferation and differentiation during embryonic development (Thomas 2000). Mice homozygous for the KAT6B mutant allele have severe defects in cerebral cortex development resulting from a severe reduction in both proliferation and differentiation of specifically the cortical progenitor population during embryonic development. KAT6B is required for the maintenance of the adult neural stem cell population and is part of a system regulating differentiation of stem cells into neurons (Merson 2006). KAT6B is also mutated in rare forms of leukaemia (Vizmanos 2003).

The MOZ locus ranks as the 12th most commonly amplified region across all cancer types (Zack 2013). MOZ is within the 8p11-p12 amplicon, which is seen at frequencies around 10-15% in various cancers, especially breast and ovarian (Turner-Ivey 2014). MOZ was first identified as a fusion partner of the CREB-binding protein (CBP) during examination of a specific chromosomal translocation in acute myeloid leukaemia (AML) (Avvakumov 2007; Borrow 1996). MOZ KAT activity is necessary for promoting the expression of MEIS1 and HOXa9, proteins that are typically seen overexpressed in some lymphomas and leukaemias. Increased survival of MOZ^{+/-} heterozygote mice in the Eμ-Myc transgenic model of B-cell lymphoma is seen, where loss of a single MOZ allele leads to a biologically relevant reduction in Meis1 and Hoxa9 levels in pre-B-cells (Sheikh 2015).

Inhibitors of some MYSTs are known. For example, the following Anacardic acid derivative is reported (Ghizzoni 2012) as inihibiting TIP60 (IC₅₀=74 μM) and MOF (IC₅₀=47 μM):

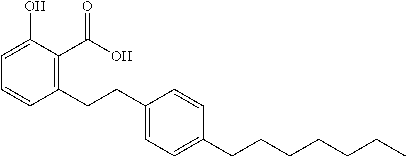 [SMILES: CCCCCCCc1ccc(CCc2cccc(O)c2C(=O)O)cc1]

Other known inhibitors include (Zhang 2017):

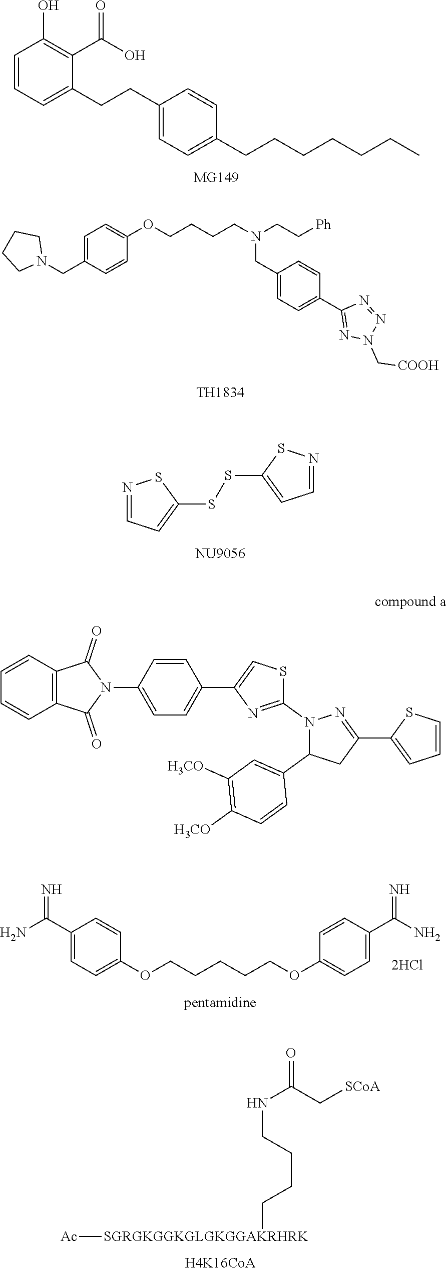

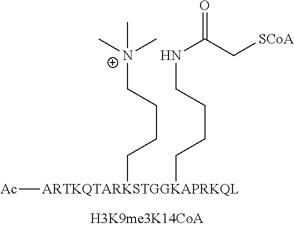

In light of the established role of KATs in general, and MYSTs in particular, in diseases such as cancer, a need exists for new inhibitors of these molecules.

### DISCLOSURE OF THE INVENTION

The present invention provides compounds which inhibit the activity of one or more KATs of the MYST family, i.e., TIP60, KAT6B, MOZ, HBO1 and MOF.

A first aspect of the present invention provides a compound of formula I:

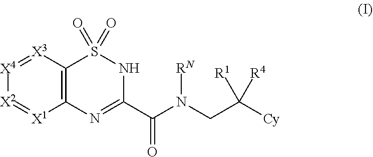

wherein:

R^{N} is H or Me;

X⁴ is selected from CY and N;

X¹, X² and X³ are each selected from CH and N, where none or one of X¹, X², X³ and X⁴ are N; Y is selected from the group consisting of: H; halo; cyano; R², where R² is selected from CH₃, CH₂F, CHF₂ and CF₃; ethynyl; cyclopropyl; OR³, where R³ is selected from H, CH₃, CH₂F, CHF₂ and CF₃; NR^{N1}R^{N2}, where R^{N1} and R^{N2} are independently selected from H and CH₃; COQ¹, where Q¹ is selected from C₁₋₄ alkyl, OH, OC₁₋₄ alkyl and NR^{N1}R^{N2}; NHSO₂Q³, where Q³ is C₁₋₃ alkyl; pyridyl; C₅ heteroaryl, which may be substituted by a group selected from C₁₋₃ alkyl, which itself may be substituted by OH or CONR^{N1}R^{N2}; SO₂Me; C₁₋₃ alkyl, substituted by NHZ, where Z is H, Me, SO₂Me, or COMe; C₁₋₃ alkyl, substituted by OH; Cy is selected from pyridyl, oxazolyl, cyclohexyl and optionally substituted phenyl, where the optional substituents are selected from the group consisting of: R²; OR⁵, where R⁵ is selected from H, CH₃, CH₂F, CHF₂, CF₃ and cyclopropyl; benzyloxy; halo; cyano; amino; C₅ heteroaryl, optionally substituted by methyl, CH₂OH, CH₂OCH₃ or ═O; phenyl; pyridyl, optionally substituted with methyl; COQ⁵, where Q⁵ is selected from OH, OCH₃ and NR^{N1}R^{N2}; and CH₂OQ⁶, where Q⁶ is H or Me;

R¹ is selected from the group consisting of: F; phenyl; pyridyl; C₅ heteroaryl, optionally substituted by methyl, CH₂OCH₃, CH₂CF₃, CHF₂, NH₂, or ═O; C₉ heteroaryl; OH; OMe; OPh; COQ⁴, where Q⁴ is selected from OH, C₁₋₃ alkyloxy, NR^{N5}R^{N6}, where R^{N5} is selected from H and Me, and R^{N5} is selected from C₁₋₄ alkyl, which itself may be substituted by CONHMe, or where R^{N5} and R^{N6} together with the N atom to which they are bound form a C₄₋₆ N-containing heterocyclyl group, (CH₂)ₙ₁CONR^{N7}R^{N8}, where n1 is 1 to 3, and R^{N7} and R^{N8} are independently selected from H and Me, and O(CH₂)ₙ₂CONR^{N9}R^{N10}, where n2 is 1 or 3. And R^{N9} and R^{N10} are independently selected from H and Me; (CH₂)ₙOQ⁷, where n is 1 or 2 and Q⁷ is H or Me; NHCO₂Q⁸, where Q⁸ is C₁₋₃ alkyl; OCONR^{N5}R^{N6};

R⁴ is selected from H, F and methyl; or

R¹ and R⁴ together with the carbon atom to which they are bound may form a C₄₋₆ cycloalkyl; and

when Cy is cyclohexyl, pyridyl or substituted phenyl, R¹ may additionally be selected from H.

A second aspect of the present invention provides a compound of the first aspect for use in a method of therapy. The second aspect also provides a pharmaceutical composition comprising a compound of the first aspect and a pharmaceutically acceptable excipient.

A third aspect of the present invention provides a method of treatment of cancer, comprising administering to a patient in need of treatment, a compound of the first aspect of the invention or a pharmaceutical composition of the first aspect of the invention. The third aspect of the present invention also provides the use of a compound of the first aspect of the invention in the manufacture of a medicament for treating cancer, and a compound of the first aspect of the invention or pharmaceutical composition thereof for use in the treatment of cancer.

As described below, the compound of the first aspect may be administered simultaneously or sequentially with radiotherapy and/or chemotherapy in the treatment of cancer.

A third aspect of the present invention provides the synthesis of compounds of the first aspect of the invention, as described below.

### Definitions

C₅₋₉ heteroaryl: The term “C₅₋₉ heteroaryl” as used herein, pertains to a monovalent moiety obtained by removing a hydrogen atom from an aromatic structure having from 5 to 9 rings atoms, of which from 1 to 3 are ring heteroatoms. The term ‘aromatic structure’ is used to denote a single ring or fused ring systems having aromatic properties, and the term ‘ring heteroatom’ refers to a nitrogen, oxygen or sulphur atom.

In this context, the prefixes (e.g. C₅₋₉, C₅, etc.) denote the number of atoms making up the aromatic structure, or range of number of atoms making up the aromatic structure, whether carbon atoms or heteroatoms.

Examples of C₅₋₉ heteroaryl structures include, but are not limited to, those derived from:

N₁: pyrrole (azole) (C₅), pyridine (azine) (C₆); pyridone (C₆); indole (C₉);

O₁: furan (oxole) (C₅);

S₁: thiophene (thiole) (C₅);

N₁O₁: oxazole (C₅), isoxazole (C₅), isoxazine (C₆);

N₂O₁: oxadiazole (furazan) (C₅);

N₁S₁: thiazole (C₅), isothiazole (C₅);

N₂S₁: thiadiazole (C₅)

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆); benzimidazole (C₉)

N₃: triazole (C₅), triazine (C₆).

Halo: The term “halo” as used herein, refers to a group selected from fluoro, chloro, bromo and iodo.

Cyano: The term “cyano” as used herein, refers to a group —C≡N.

C₁₋₄ alkyl: The term “C₁₋₄ alkyl” as used herein, pertains to a monovalent moiety obtained by removing a hydrogen atom from a carbon atom of a saturated hydrocarbon compound having from 1 to 4 carbon atoms.

Examples of saturated alkyl groups include, but are not limited to, methyl (C₁), ethyl (C₂), propyl (C₃), and butyl (C₄).

Examples of saturated linear alkyl groups include, but are not limited to, methyl (C₁), ethyl (C₂), n-propyl (C₃), and n-butyl (C₄).

Examples of saturated branched alkyl groups include iso-propyl (C₃), iso-butyl (C₄), sec-butyl (C₄) and tert-butyl (C₄).

C₄₋₆ heterocyclyl: The term “C₄₋₆ heterocyclyl” as used herein, pertains to a monovalent moiety obtained by removing a hydrogen atom from a ring atom of a monocyclic heterocyclic compound, which moiety has from 4 to 6 ring atoms; of which from 1 to 2 atoms are heteroatoms, chosen from oxygen or nitrogen.

In this context, the prefixes (e.g. C₄₋₆) denote the number of ring atoms, or range of number of ring atoms, whether carbon atoms or heteroatoms.

Examples of C₄₋₆ heterocyclyl groups include, but are not limited to, those derived from:

N₁: azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

N₂: diazetidine (C₄), imidazolidine (C₅), pyrazolidine (diazolidine) (C₅), imidazoline (C₅), pyrazoline (dihydropyrazole) (C₅), piperazine (C₆);

O₁: oxetane (C₄), tetrahydrofuran (C₅); oxane (C₆);

O₂: dioxetane (C₄), dioxolane (C₅); dioxane (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆).

Where the C₄₋₆ heterocyclyl is defined as being “N-containing” this means one of the ring atoms is N, such that the group may be selected from:

N₁: azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

N₂: diazetidine (C₄), imidazolidine (C₅), pyrazolidine (diazolidine) (C₅), imidazoline (C₆), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆).

Benzyloxy: —OCH₂-Phenyl.

Includes Other Forms

Unless otherwise specified, included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms.

Salts

It may be convenient or desirable to prepare, purify, and/or handle a corresponding salt of the active compound, for example, a pharmaceutically-acceptable salt. Examples of pharmaceutically acceptable salts are discussed in Berge 1977.

For example, if the compound is anionic, or has a functional group which may be anionic (e.g. —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e. NH₄⁺) and substituted ammonium ions (e.g. NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g. —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, trifluoroacetic acid and valeric. Examples of suitable polymeric organic anions include, but are not limited to, those derived from the following polymeric acids: tannic acid, carboxymethyl cellulose.

Solvates

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

Isomers

Certain compounds of the invention may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

In the present invention, the carbon atom to which R¹ and Cy are bound may be a stereochemical centre, i.e. when R¹ is not H and R¹ and Cy are different. The compounds of the present invention may be a racemic mixture, or may be in enantiomeric excess or substantially enantiomerically pure.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g. C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

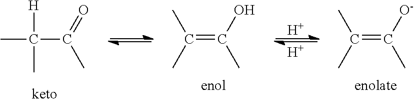

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Note that specifically included in the term “isomer” are compounds with one or more isotopic substitutions. For example, H may be in any isotopic form, including ¹H, ²H (D), and ³H (T); C may be in any isotopic form, including ¹²C, ¹³O, and ¹⁴C; O may be in any isotopic form, including ¹⁶O and ¹⁸O; and the like.

Examples of isotopes that can be incorporated into compounds of the invention include isotopes of hydrogen, carbon, nitrogen, oxygen, phosphorous, fluorine, and chlorine, such as, but not limited to ²H (deuterium, D), ³H (tritium), ¹¹C, ¹³C, ¹⁴C, ¹⁵N, ¹⁸F, ³¹F, ³²F, ³⁵S, ³⁶Cl, and ¹²⁵I. Various isotopically labeled compounds of the present invention, for example those into which radioactive isotopes such as 3H, 13C, and 14C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays, or in radioactive treatment of patients. Deuterium labelled or substituted therapeutic compounds of the invention may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life or reduced dosage requirements. An 18F labeled compound may be useful for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent. Further, substitution with heavier isotopes, particularly deuterium (i.e., 2H or D) may afford certain therapeutic advantages resulting from greater metabolic stability, for example increased in vivo half-life or reduced dosage requirements or an improvement in therapeutic index. It is understood that deuterium in this context is regarded as a substituent. The concentration of such a heavier isotope, specifically deuterium, may be defined by an isotopic enrichment factor. In the compounds of this invention any atom not specifically designated as a particular isotope is meant to represent any stable isotope of that atom.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Inhibition

The compounds of the present invention inhibit the activity of one or more KATs of the MYST family, i.e., TIP60, KAT6B, MOZ, HBO1 and MOF.

The inhibitory activity of the compounds of the invention is likely to vary between the KATs of the MYST family.

The compounds of the present invention may selectively inhibit the activity of one or more KATs of the MYST family over other KATs of the MYST family, i.e. the inhibitory activity of the compound may be higher for one or more of the KATs of the MYST family over one or more of the other KATs of the MYST family.

Compounds of the present invention may (selectively) inhibit the activity of a single HAT of the MYST family. Thus, compounds of the present invention may inhibit the activity of TIP60, MORF, MOZ, HBO1 or MOF.

Compounds of the present invention may inhibit the activity of two KATs of the MYST family, for example TIP60 and HBO1.

Compounds of the present invention may inhibit the activity of three KATs of the MYST family, for example TIP60, HBO1 and MOF.

Compounds of the present invention may inhibit the activity of four KATs of the MYST family, for example TIP60, HBO1, MOF and MOZ.

Compounds of the present invention may inhibit the activity of all five KATs of the MYST family, thus the compounds may inhibit the activity of TIP60, KAT6B, MOZ, HBO1 and MOF.

Therapeutic Indications

Compounds disclosed herein may provide a therapeutic benefit in a number of disorders, in particular, in the treatment or prevention of cancers.

Cancer

Inhibitors of post-translational lysine acetylation mediated by KATs of the MYST family are considered to be promising anti-neoplastic agents and therefore may be useful therapeutic agents, e.g. for use in the treatment of cancer. Such agents may also be useful as therapeutic agents for the treatment of cancers which exhibit overexpression of MYST proteins.

A “cancer” may be any form of cancer. In particular, a cancer can comprise any one or more of the following: leukemia, acute lymphocytic leukemia (ALL), acute myeloid leukemia (AML), chronic lymphocytic leukemia (CLL), chronic myeloid leukemia (CML), non-Hodgkin's lymphoma, Hodgkin's disease, prostate cancer, lung cancer, melanoma, breast cancer, colon and rectal cancer, colon cancer, squamous cell carcinoma and gastric cancer.

Alternatively, the cancer may comprise adrenocortical cancer, anal cancer, bladder cancer, blood cancer, bone cancer, brain tumor, cancer of the female genital system, cancer of the male genital system, central nervous system lymphoma, cervical cancer, childhood rhabdomyosarcoma, childhood sarcoma, endometrial cancer, endometrial sarcoma, esophageal cancer, eye cancer, gallbladder cancer, gastrointestinal tract cancer, hairy cell leukemia, head and neck cancer, hepatocellular cancer, hypopharyngeal cancer, Kaposi's sarcoma, kidney cancer, laryngeal cancer, liver cancer, malignant fibrous histiocytoma, malignant thymoma, mesothelioma, multiple myeloma, myeloma, nasal cavity and paranasal sinus cancer, nasopharyngeal cancer, nervous system cancer, neuroblastoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, rectal cancer, respiratory system, retinoblastoma, salivary gland cancer, skin cancer, small intestine cancer, soft tissue sarcoma, stomach cancer, stomach cancer, testicular cancer, thyroid cancer, urinary system cancer, uterine sarcoma, vaginal cancer, vascular system, Waldenstrom's macroglobulinemia and/or Wilms' tumor.

Cancers may be of a particular type. Examples of types of cancer include lymphoma, melanoma, carcinoma (e.g. adenocarcinoma, hepatocellular carcinoma, medullary carcinoma, papillary carcinoma, squamous cell carcinoma), astrocytoma, glioma, medulloblastoma, myeloma, meningioma, neuroblastoma, sarcoma (e.g. angiosarcoma, chrondrosarcoma, osteosarcoma).

The cancer may be a MYST overexpressing cancer. The cancer may over-express MYST protein relative to non-cancerous tissue. In some cases, the cancer overproduces MYST mRNA relative to non-cancerous tissue. The overexpressed MYST protein or MYST mRNA may be any one KATs of the MYST family, i.e. any one of TIP60, KAT6B, MOZ, HBO1 and MOF. In some embodiments, the cancer may overexpress more than one KATs of the MYST family, e.g. two or more selected from the group consisting of TIP60, KAT6B, MOZ, HBO1 and MOF. The cancer may be a cancer that evades immune recognition, e.g. via tumor-associated Treg cells.

Alternatively or additionally, the cancer may be a bromodomain overexpressing cancer: The cancer cell may overexpress one or more bromodomain-containing proteins (herein referred to as “bromodomain proteins”) relative to non-cancerous tissue. It may overproduce one or more bromodomain mRNA as compared to non-cancerous tissue. In some cases, the level of bromodomain protein and/or mRNA in the cell is at a level approximately equivalent to that of a non-cancerous cell. The cancer may overexpress one or more bromodomain proteins selected from the group consisting of; a bromodomain protein (namely BRD2, BRD3, BRD4, BRD7, BRD8, BRD9 and BRDT), TAF1/TAF1L, TFIID, SMARC2 (also called BRM) and SMARC4 (also called BRG1). For example, some colon cancers overexpress BRD8. Some acute myeloid leukemia cells overexpress BRD4.

Treg Cells as a Cancer Target

Treg cells are immunosuppressive cells, which act to prevent autoimmunity in the healthy mammalian immune system. However, some cancers act to upregulate Treg activity to evade the host immune system. Infiltration of Tregs in many tumour types correlates with poor patient prognoses and Treg cell depletion in tumour models demonstrates increased anti-tumour immune responses (Melero 2015). Tumour-associated Treg suppression of the host immune system has been reported in lung (Joshi 2015), (Tso 2012), breast (Gobert 2009; Yan 2011), prostate (Miller 2006) & pancreatic (Wang X 2016) cancers. FOXP3 is considered to be the master regulator of Treg differentiation, development and function of Treg cells.

Several studies have demonstrated that acetylation of FOXP3 plays a critical role in the stability of the FOXP3 protein and in regulating its ability to access DNA; and FOXP3 acetylation is mediated by KATs (Dhuban 2017). Decreases in TIP60-mediated FOXP3 acetylation has been shown to attenuate Treg development, suggesting a further mechanism by which the inhibition of the acetylating activity of MYST proteins could be used to intervene in diseases such as cancer.

Combination Therapies

The agents described herein may be useful in combination with other anti-cancer therapies. They may act synergistically with chemo- or radiotherapy, and/or with bromodomain targeted drugs. For example, the agents described herein may be useful in combination with a BET inhibitor. BET inhibitors reversibly bind the bromodomains of the BET proteins BRD2, BRD3, BRD4 and BRDT.

Inhibition of HAT proteins of the MYST family, to reduce the extent of lysine acetylation of histones (and other nuclear proteins described herein) will likely sensitize tumour cells to chemo- and radiotherapy by attenuating the process of DNA damage repair, e.g. the repair of DNA double-strand breaks (DSB), thus increasing the frequency of chemo- and radiotherapy induced cancer cell death. Therefore, it is likely that inhibition of HAT proteins of the MYST family would synergize well with low dose chemo- or radiotherapy.

Thus, in some cases, a MYST protein antagonist disclosed herein may be administered in conjunction with a radiotherapeutic or chemotherapeutic regime. It may be administered simultaneously or sequentially with radio and/or chemotherapy. Suitable chemotherapeutic agents and radiotherapy protocols will be readily appreciable to the skilled person. In particular, the compound described herein may be combined with low dose chemo or radio therapy. Appropriate dosages for “low dose” chemo or radio therapy will be readily appreciable to the skilled practitioner.

In particular, where the compounds of the present application are used to abrogate Treg suppression, these may be combined with immune checkpoint inhibitors (Melero 2015, Wang L 2016). Furthermore, where compounds of the present invention which abrogate Treg suppression may be used in combination with radiotherapy, to reduce the depletion of Treg function in tumours (Persa 2015, Jeong 2016)

Methods of Treatment

The compounds of the present invention may be used in a method of therapy. Also provided is a method of treatment, comprising administering to a subject in need of treatment a therapeutically-effective amount of a compound of the invention. The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

As described above, the anti-cancer treatment defined herein may be applied as a sole therapy or may involve, in addition to the compound of the invention, conventional surgery or radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cisplatin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5 fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and docetaxel (Taxotere) and polokinase inhibitors); and topoisomerase inhibitors (for example epipodophyllotoxins like etoposide and teniposide, amsacrine, topotecan and camptothecin);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5*-reductase such as finasteride;

(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661 and 4-((2,4-dichloro-5-methoxyphenyl)amino)-6-methoxy-7-(3-(4-methylpiperazin-1-yl)propoxy)quinoline-3-carbonitrile (bosutinib, SKI-606; Cancer research (2003), 63(2), 375-81), and metalloproteinase inhibitors like marimastat, inhibitors of urokinase plasminogen activator receptor function or antibodies to Heparanase);

(iv) inhibitors of growth factor function: for example such inhibitors include growth factor antibodies and growth factor receptor antibodies (for example the anti erbB2 antibody trastuzumab [HerceptinT], the anti-EGFR antibody panitumumab, the anti erbB1 antibody cetuximab [Erbitux, C225] and any growth factor or growth factor receptor antibodies disclosed by Stern 2005; such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI 774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;

(v) antiangiogenic and antilymphangiogenic agents such as those which inhibit the effects of vascular endothelial growth factor, [for example the anti vascular endothelial cell growth factor A (VEGFA) antibody bevacizumab (AvastinT), the anti vascular endothelial cell growth factor A (VEGFA) antibody ranibizumab, the anti-VEGF aptamer pegaptanib, the anti vascular endothelial growth factor receptor 3 (VEGFR3) antibody IMC-3C5, the anti vascular endothelial cell growth factor C (VEGFC) antibody VGX-100, the anti vascular endothelial cell growth factor D (VEGFD) antibody VGX-200, the soluble form of the vascular endothelial growth factor receptor 3 (VEGFR3) VGX-300 and VEGF receptor tyrosine kinase inhibitors such as 4-(4-bromo-2-fluoroanilino)-6-methoxy-7-(1-methylpiperidin-4-ylmethoxy)quinazoline (vandetanib; ZD6474; Example 2 within WO 01/32651), 4-(4-fluoro-2-methylindol-5-yloxy)-6-methoxy-7-(3-pyrrolidin-1-ylpropoxy)quinazoline (cediranib; AZD2171; Example 240 within WO 00/47212), vatalanib (PTK787; WO 98/35985), pazopanib (GW786034), axitinib (AG013736), sorafenib and sunitinib (SU11248; WO 01/60814), compounds such as those disclosed in International Patent Applications WO97/22596, WO 97/30035, WO 97/32856 and WO 98/13354 and compounds that work by other mechanisms (for example linomide, inhibitors of integrin avb3 function and angiostatin)];

(vi) vascular damaging agents such as Combretastatin A4 and compounds disclosed in International Patent Applications WO 99/02166, WO 00/40529, WO 00/41669, WO 01/92224, WO 02/04434 and WO 02/08213;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene directed enzyme pro drug therapy) approaches such as those using cytosine deaminase, thymidine kinase or a bacterial nitroreductase enzyme and approaches to increase patient tolerance to chemotherapy or radiotherapy such as multi drug resistance gene therapy; and

(ix) immunotherapy approaches, including for example ex vivo and in vivo approaches to increase the immunogenicity of patient tumour cells, such as transfection with cytokines such as interleukin 2, interleukin 4 or granulocyte macrophage colony stimulating factor, approaches to decrease T cell anergy, approaches using transfected immune cells such as cytokine transfected dendritic cells, approaches using cytokine transfected tumour cell lines and approaches using anti idiotypic antibodies

Administration

The active compound or pharmaceutical composition comprising the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intravitreal and intrasternal; by implant of a depot, for example, subcutaneously, intravitreal or intramuscularly. The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-utan, gibbon), or a human.

Formulations

While it is possible for the active compound to be administered alone, it is preferable to present it as a pharmaceutical composition (e.g. formulation) comprising at least one active compound, as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilisers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, losenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or moulding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, sorbic acid). Moulded tablets may be made by moulding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavoured basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required.

Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g. by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/mL to about 10 μg/mL, for example from about 10 ng/ml to about 1 μg/mL. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Dosage

It will be appreciated by one of skill in the art that appropriate dosages of the compound, and compositions comprising the compound, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the active compound is in the range of about 100 ng to about 25 mg (more typically about 1 μg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

In one embodiment, the active compound is administered to a human patient according to the following dosage regime: about 100 mg, 3 times daily.

In one embodiment, the active compound is administered to a human patient according to the following dosage regime: about 150 mg, 2 times daily.

In one embodiment, the active compound is administered to a human patient according to the following dosage regime: about 200 mg, 2 times daily.

However in one embodiment, the active compound is administered to a human patient according to the following dosage regime: about 50 or about 75 mg, 3 or 4 times daily.

In one embodiment, the active compound is administered to a human patient according to the following dosage regime: about 100 or about 125 mg, 2 times daily.

Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

The Subject/Patient

The subject/patient may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus. In one preferred embodiment, the subject/patient is a human.

General Synthesis Methods

The compounds of the invention can be prepared employing the following general methods and using procedures described in detail in the examples. The reaction conditions referred to are illustrative and non-limiting, for example one skilled in the art may use a diverse range of synthetic methods to synthesize the desired compounds such as but not limited to methods described in literature (for example but not limited to March's Advanced Organic Chemistry: Reactions, Mechanisms, and Structure, 7th Edition or Larock's Comprehensive Organic Transformations: Comprehensive Organic Transformations: A Guide to Functional Group Preparations).

Compounds of formula I, as described above, can be prepared by synthetic strategies outlined below, wherein the definitions above apply. The synthetic strategies could be applied to the use of racemic or single enantiomer starting materials.

General Synthesis Method 1

Scheme 1A illustrates the formation of the amide bond by coupling the relevant benzothiadiazinedioxide alkyl ester G1 (R¹⁰=alkyl) with primary amine G2. Methods to form such amides G3 will be apparent to those skilled in the art, but include for example the use of microwave irradiation or conventional heating, either in a reagent-free fashion or with reagents such as NEt₃, DMAP or DIPEA and optionally with the use of a suitable solvent, e.g. ethanol or acetonitrile.

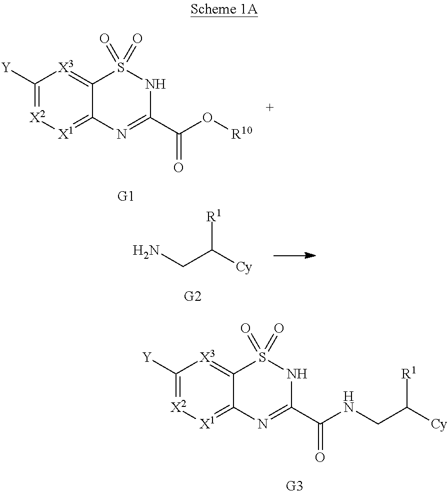

General Synthesis Method 2

Scheme 2A illustrates the formation of the amide bond by coupling the relevant benzothiadiazinedioxide carboxylic acid G4 to primary amine G2. Methods to form such amides G3 will be apparent to those skilled in the art, but include for example, the use of reagents such as EDCIl/DMAP, EDCIl/HOBt, HATU, HBTU and T3P. Alternatively the acid can be activated prior to treatment with the primary amine G2. Such methods include, but are not limited to, acyl chloride formation from G4 (e.g. SOCl₂, POCl₃, oxalyl chloride and DMF in an appropriate solvent), mixed anhydride formation from G4 (ClCO₂CH₃ and Et₃N, iso-butylO₂CCl and Et₃N in an appropriate solvent, e.g. CH₂Cl₂ or MeCN) or acyl imidazolide formation (carbonyl diimidazole and DIPEA in an appropriate solvent).

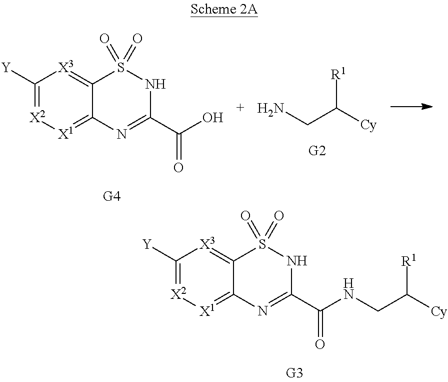

General Synthesis Method 3

Scheme 3A illustrates the formation of the benzothiadiazinedioxide core G1 by acylation of the aminobenzenesulfonamide G5 with ethyl 2-chloro-2-oxoacetate, followed by cyclization of G6 with a base such as sodium hydride to form core G1.

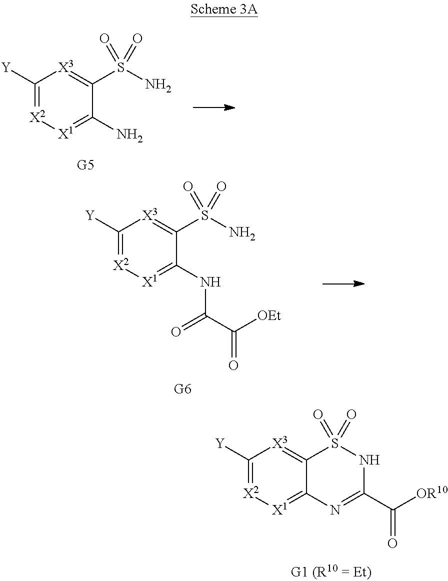

Alternatively G5 can be treated with a reagent such as ethyl carbonocyanidate to form the bicyclic core G1 directly (Scheme 4A).

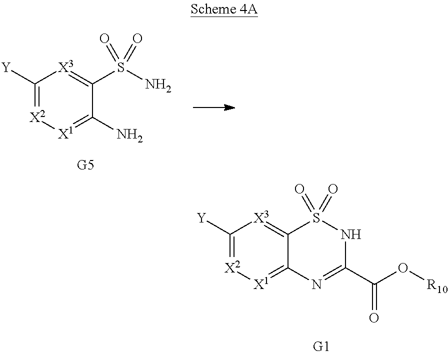

Formation of G5 (Y═Cl, Br or I) can be achieved from G5 (Y═H) using reagents such as N-chlorosuccinimide, Br₂ or ICI, which can then undergo cyclisation to give G1 as shown in Scheme 3A or 4A.

General Synthesis Method 4

Scheme 5A illustrates the formation of primary amines G2 from common intermediate G10. Preparation of versatile intermediate G10 can be achieved through the alkylation of benzylacetate G8 with an alkyl halide, e.g. G7 (where PG is an appropriate protecting group), using a strong base such as LiHMDS followed by the hydrogenation of ester G9. Alternative preparation of G10 can be achieved through the N-protection of an appropriate beta amino acid. Carboxylic acid G10 is a versatile intermediate that can be used to introduce a range of R¹ substituents. Formation of an oxazole can be achieved through activation to the acyl chloride and then treatment with 1,2,3-triazole in sulfolane. Likewise, treatment of the acyl chloride with a suitable hydrazide (e.g. formyl hydrazine), followed by Burgess reagent will furnish a 1,3,4-oxadiazole. The synthesis of other aromatic heterocycles from G10 can be achieved by those skilled in the art, using methods described in Hereocyclic Chemistry (J. A. Joule and K. Mills, Blackwell Science). Carboxylic acid G10 can be converted to amides using a suitable primary or secondary amine and an appropriate coupling agent (e.g. T3P, HATU, HBTU, EDCI, etc.). Curtius rearrangement can be achieved through treatment of carboxylic acid G10 with an appropriate azido reagent, e.g. DPPA. The resulting isocyanate can be trapped with a suitable alcohol to give a carbamate. If a Boc-protected amine is introduced, the protecting group can be removed to furnish a primary amine, which itself could be further derivatised using methods known to those skilled in the art.

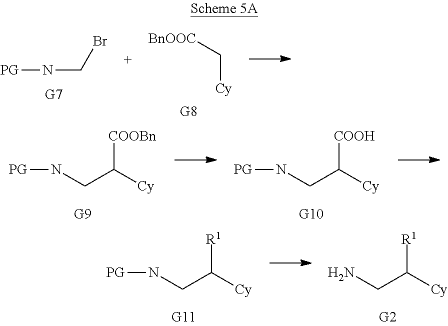

Deprotection of these materials G11 yields primary amines G2, which can then be coupled following general synthesis methods 1 or 2. Conditions for the removal of the protecting group are dependent on the type of protecting group employed, and may include but are not limited to such methods as acid or base hydrolysis, transition metal catalysed cleavage and hydrogenation over transition metal catalysts. Other suitable protecting groups and removal methods will be known to those skilled in the art (for example Greene's Protective Groups in Organic Synthesis, 4th Edition). The use of such a protecting group could be relevant in the other Schemes described.

General Synthesis Method 5

Scheme 6A shows the conversion of intermediate G12 (where R¹⁰ is alkyl or H) and R¹¹ is a halogen (e.g. I, Br or Cl) to G1 with a range of substituents Y. Suzuki coupling from G12 can be used to introduce heteroaromatic rings through the use of an appropriate boronic acid or boronate ester and an appropriate catalyst (e.g. Pd^{II} or Pd⁰) optionally with a suitable ligand. Y═CN can be introduced through treatment of G12 with a suitable source of cyanide using an appropriate catalyst and ligand. An ester can be introduced to Y using a carbonylation reaction, using carbon monoxide gas, a suitable alcohol (e.g. ethanol) and a suitable catalyst. The alkyl ester can be hydrolysed to give a carboxylic acid (e.g. using LiOH is a suitable solvent) and then couple with a suitable amine to form an amide using a coupling reagent (e.g. T3P, HATU, HBTU etc). Intermediates G1 can be converted to G3, for example by using general synthesis methods 1 or 2.

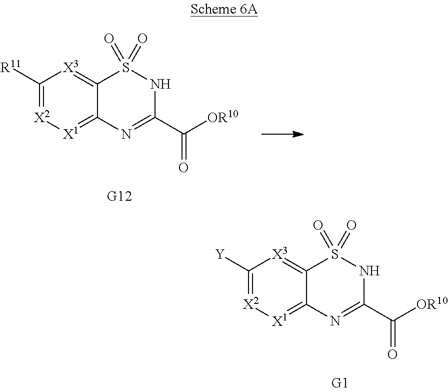

General Synthesis Method 6

Scheme 7A illustrates an alternative route for accessing primary amines (X═CH or N). The conversion of a suitable halophenyl or halopyridyl compound G13 to G14 can be achieved as shown in Scheme 7A. If the halogen in G13 is iodo or bromo, an N-linked 5-membered aromatic heterocycle R¹² can be introduced with the use of a suitable copper catalyst. Where R¹² is a C-linked heterocycle, an appropriate boronic acid or boronate ester in combination with a suitable catalyst (e.g. Pd^{II} or Pd⁰), can effect the formation of G14. Where the halogen is F or Cl, treatment of G13 with a suitable nucleophile (e.g. an alcohol or 5-membered heterocycle, e.g. pyrazole or triazole), an S_{N}Ar reaction could effect the formation of R¹²═OR³, or N-linked 5-membered aromatic heterocycle. Reduction of the nitrile group in G14 with a suitable reducing agent, e.g. LiAlH₄ or BH₃ effects the formation of primary amine G15, which can be converted to G3 using the general synthesis methods 1 or 2.

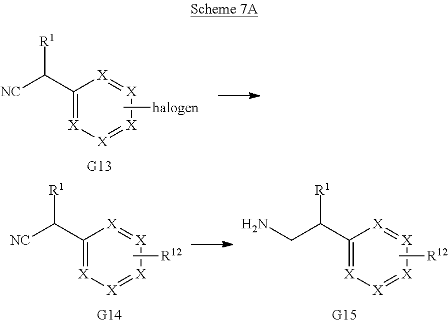

An alternative to the use of the nitrile shown in Scheme 7A, is shown in Scheme 8A, where PG is a suitable protecting group or a hydrogen atom. Such protecting groups include, but are not limited to, phthalimide, Boc, acetyl, CBZ, benzyl and dimethoxy benzyl. Halogen G16 can be converted to G17 using similar methods to those described for G13 to G14. Deprotection of G17 to give G18 can be achieved using methods known to those skilled in the art.

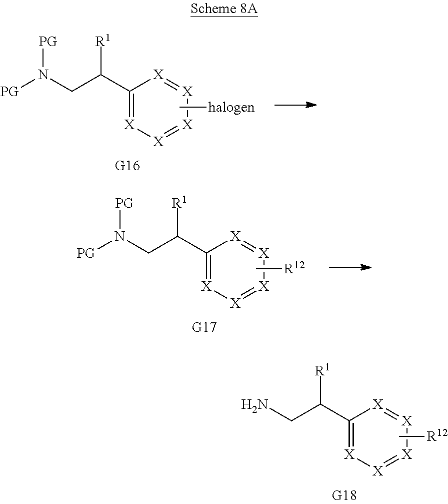

General Synthesis Method 7

Scheme 8B illustrates an alternative route for accessing primary amine G2. Alkylation of structure G19 can be achieved with an alkyl halide, e.g. G7 (where PG is an appropriate protecting group), using an appropriate base such as but not limited to LiHMDS. Deprotection of G11 yields primary amines G2, which may then be coupled following general synthesis methods 1 or 2.

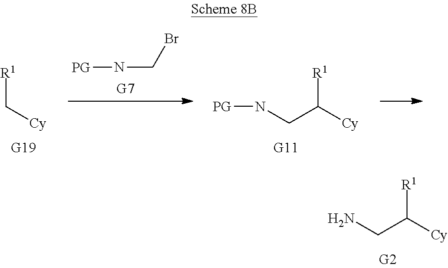

General Synthesis Method 8

Scheme 9A illustrates the introduction of substituent Z on the benzylic carbon in structure G19 to form the corresponding structure G20. Substituent Z may be but is not limited to a halogen such as fluoro. For example, G19 may be reacted with a suitable base such as for example LiHMDS to form the corresponding carbanion which may be treated with a suitable source of F⁺ such as but not limited to NFSI (N-fluorodibenzenesulfonimide).

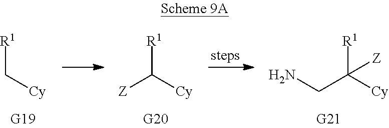

Subsequent alkylation and deprotection of G20 described as described in general synthesis method 7 would give amine G21, which may then be coupled following general synthesis methods 1 or 2.

General Synthesis Method 9

Scheme 10A and B illustrate the synthesis of a primary amine G24 (where R¹³ represents a suitable substituent, including H) from starting material G22 (where X═OH or halogen such as but not limited to Br or activated alcohol such as but not limited to mesylate), for example via intermediate G23 in the Gabriel synthesis (Scheme 10A) or via the azide intermediate G25 (Scheme 10B).

The formation of intermediate G23 may be achieved via nucleophilic substitution or via the Mitsunobu reaction (when X═OH). Cleavage to give amine G24 may be achieved by treating G23 with for example hydrazine.

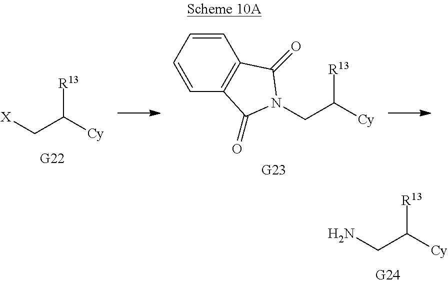

The azide G25 may be achieved via for example nucleophilic substitution or Mitsunobu and then reduced to the primary amine by methods known to someone skilled in the art but may include the use of a metal catalyst in the presence of hydrogen or the use of triphenylphosphine (Staudinger reaction).

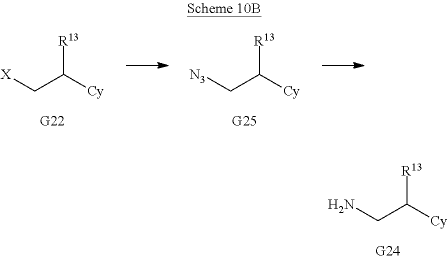

General Synthesis Method 10

Scheme 11A illustrates the formation of primary amine G28 via alkylation of a nitrile such as G26. Groups R¹⁴ may be alkyl groups such as but not limited to methyl or ethyl and may connected to form for example a cyclopentyl or cyclohexyl moiety. Methods to form intermediate G27 from G26 may be known to someone skilled in the art and include the use of an appropriate base such as hydroxide or an alkoxide base to form an anion which is then reacted with for example an alkyl halide. If the two R14 groups form a cycle, the appropriate starting material may be a dihaloalkane such as for example 1,4-dibromobutane to form the cyclopentyl moiety.

Subsequent reduction of the nitrile in structure G27 may be achieved via hydrogenation in the presence of a metal catalyst.

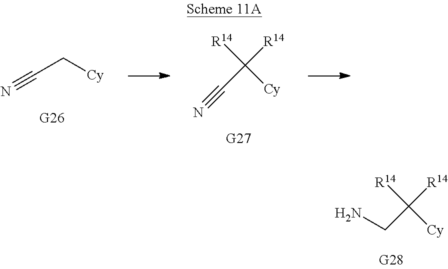

Further Preferences

The following preferences may apply to all aspects of the invention as described above, or may relate to a single aspect. The preferences may be combined together in any combination.

R^{N}

In some embodiments, R^{N} is H.

In some embodiments, R^{N} is Me.

X⁴

In some embodiments, X⁴ is CY.

In some embodiments, X⁴ is N.

X¹, X² and X³

In some embodiments, none of X¹, X² and X³ are N, i.e. they are all CH.

In some embodiments, none of X¹, X², X³ and X⁴ are N.

In some embodiments, X¹ is N.

In some embodiments, X² is N.

In some embodiments, X³ is N.

Compounds where none of X¹, X², X³ and X⁴ are N may be preferred for compounds which inhibit TIP60.

Y

In some embodiments, Y is H.

In some embodiments, Y is halo. When Y is halo, it may be selected from I and F. In some of these embodiments, Y is F. In other of these embodiments, Y is I.

In some embodiments, Y is cyano (C≡N).

In some embodiments, Y is R². In some of these embodiments, R² is CH₃ (methyl). In other of these embodiments, R² is CH₂F. In other of these embodiments, R² is CHF₂. In other of these embodiments, R² is CF₃.

In certain embodiments, R² may be selected from CH₃ and CF₃.

In some embodiments, Y is ethynyl (C≡CH).

In some embodiments, Y is cyclopropyl.

In some embodiments, Y is OR³. In some of these embodiments, R³ is H. In other of these embodiments, R³ is CH₃ (methyl). In other of these embodiments, R³ is CH₂F. In other of these embodiments, R³ is CHF₂. In other of these embodiments, R³ is CF₃. In certain embodiments, R³ may be selected from H and CF₃.

In some embodiments, Y is NR^{N1}R^{N2}. In some of these embodiments, R^{N1} and R^{N2} are both H. In other of these embodiments, R^{N1} and R^{N2} are both Me. In other of these embodiments, R^{N1} is H and R^{N2} is Me.

In some embodiments, Y is COQ¹. In some of these embodiments, Q¹ is C₁₋₄ alkyl, such as methyl. In other of these embodiments, Q¹ is OH. In other of these embodiments, Q¹ is OC₁₋₄ alkyl, such as OMe. In other of these embodiments, Q¹ is NR^{N1}R^{N2}. In some of these particular embodiments, R^{N1} and R^{N2} are both H. In other of these particular embodiments, R^{N1} and R^{N2} are both Me. In other of these particular embodiments, R^{N1} is H and R^{N2} is Me.

In certain embodiments, Y is selected from COMe, CO₂H, CO₂Me, CONH₂, CONHMe and CONMe₂.

In some embodiments, Y is NHSO₂Q³. In these embodiments, Q³ is C₁₋₃ alkyl, such as methyl.

In some embodiments, Y is pyridyl.

In some embodiments, Y is C₅ heteroaryl, which is optionally substituted. In some of these embodiments, the C₅ heteroaryl group may be selected from pyrrolyl, furanyl, thiolyl, oxazolyl, isoxazolyl, oxadiazolyl, thiazolyl, isothiazolyl, thiadiazolyl, imidazolyl, pyrazolyl or triazolyl. The C₅ heteroaryl group may be selected from those containing a nitrogen ring atom. The C₅ heteroaryl group may be selected from those containing a nitrogen ring atom and a further ring heteroatom. The C₅ heteroaryl group may be selected from thiazolyl and pyrazolyl.

The substituent group may be selected from unsubstituted C₁₋₃ alkyl, such as methyl, C₁₋₃ alkyl substituted by OH, such as C₂H₄OH, and C₁₋₃ alkyl substituted by CONR^{N1}R^{N2}, such as CH₂CONHMe.

In some embodiments, Y is SO₂Me.

In some embodiments, Y is C₁₋₃ alkyl, substituted by NHZ, where Z is H, Me, SO₂Me, or COMe. In some of these embodiments, Z is H. In other of these embodiments, Z is Me. In other of these embodiments, Z is SO₂Me. In other of these embodiments, Z is COMe. In certain of these embodiments, Y is CH(NH₂)CH₃, CH(NHCH₃)CH₃, CH(NHSO₂Me)CH₃, or CH(NHCOMe)CH₃.

In some embodiments, Y is C₁₋₃ alkyl, substituted by OH. In some of these embodiments, Y is CH(OH)CH₃.

Embodiments where Y is I or Br may be preferred for compounds which inhibit TIP60.

Embodiments where Y is I may be further preferred for compounds which inhibit TIP60.

Embodiments where Y is selected from I, Br, CN, COQ¹ (where Q¹ is NR^{N1}R^{N2}) and C₅ heteroaryl may be preferred for compounds which inhibit MOZ. Embodiments where Y is selected from CN, COQ¹ (where Q¹ is NR^{N1}R^{N2}) and C₅ heteroaryl may be further preferred for compounds which inhibit MOZ

Embodiments where Y is I or Br may be preferred for compounds which inhibit HBO1.

Embodiments where Y is Br may be further preferred for compounds which inhibit HBO1.

R¹

In some embodiments (where Cy is pyridyl, cyclohexyl or substituted phenyl), R¹ is H.

When Cy is cyclohexyl, in some embodiments R¹ may only be H if Y is present and is not H.

In some embodiments, R¹ is F.

In some embodiments, R¹ is phenyl.

In some embodiments, R¹ is pyridyl.

In some embodiments, R¹ is C₅ heteroaryl, optionally substituted by methyl, CH₂OCH₃, CH₂CF₃, CHF₂, NH₂, or ═O. In some of these embodiments, R¹ is unsubstituted C₅ heteroaryl. In others of these embodiments, R¹ is C₅ heteroaryl substituted with methyl. In others of these embodiments, R¹ is C₅ heteroaryl substituted with CH₂OCH₃. In others of these embodiments, R¹ is C₅ heteroaryl substituted with CH₂CF₃. In others of these embodiments, R¹ is C₅ heteroaryl substituted with CHF₂. In others of these embodiments, R¹ is C₅ heteroaryl substituted with NH₂. In others of these embodiments, R¹ is C₅ heteroaryl substituted with ═O.

In some of embodiments, the C₅ heteroaryl group may contain at least one nitrogen ring atom. In these embodiments, any other ring heteroatoms may be selected from nitrogen and oxygen. In certain embodiments, the C₅ heteroaryl group may be selected from pyrrolyl, oxazolyl, isoxazolyl, oxadiazolyl, imidazolyl, pyrazolyl and triazolyl. In other certain embodiments, the C₅ heteroaryl group may be selected from pyrrolyl, oxazolyl, oxadiazolyl, pyrazolyl and triazolyl.

In some embodiments, R¹ is C₉ heteroaryl. In some of these embodiments, R¹ is indolyl.

In some embodiments, R¹ is OH.

In some embodiments, R¹ is OMe

In some embodiments, R¹ is OPh.

In some embodiments, R¹ is COQ⁴, where Q⁴ is selected from OH and C₁₋₃ alkyloxy. In some of these embodiments, R¹ is CO₂H. In other of these embodiments, R¹ is CO₂Me. In other of these embodiments, R¹ is CO₂Et. In other of these embodiments, R¹ is CO₂C(CH₃)₂.

In some embodiments, R¹ is COQ⁴, where Q⁴ is NR^{N5}R^{N6}, where R^{N5} is selected from H and Me, and R^{N5} is selected from C₁₋₄ alkyl, which itself may be substituted by CONHMe, or where R^{N5} and R^{N6} together with the N atom to which they are bound form a C₄₋₆ N-containing heterocyclyl group. In some of these embodiments, R¹ is CO₂NH₂. In other of these embodiments, R¹ is CO₂NHMe. In other of these embodiments, R¹ is CO₂NMe₂. In other of these embodiments, R¹ is CO₂NHEt. In other of these embodiments, R¹ is CO₂piperidinyl.

In some embodiments, R¹ is COQ⁴, where Q⁴ is (CH₂)ₙ₁CONR^{N7}R^{N8}, where n1 is 1 to 3, and R^{N7} and R^{N8} are independently selected from H and Me. In some of these embodiments, n1 is 1. In other of these embodiments, n1 is 2. In other of these embodiments, n1 is 3. In certain embodiments, R¹ is C₃H₆CONHCH₃.

In some embodiments, R¹ is COQ⁴, where Q⁴ is O(CH₂)ₙ₂CONR^{N9}R^{N10}, where n2 is 1 or 2, and R^{N9} and R^{N10} are independently selected from H and Me. In some of these embodiments, n2 is 1. In other of these embodiments, n2 is 2. In certain embodiments, R¹ is OC₂H₄CONHCH₃.

In some embodiments, R¹ is (CH₂)ₙOQ⁷, where n is 1 or 2 and Q⁷ is H or Me. In some of these embodiments R¹ is CH₂OH. In other of these embodiments, R¹ is (CH₂)₂OH. In other of these embodiments, R¹ is CH₂OMe. In other of these embodiments, R¹ is (CH₂)₂OMe.

In some embodiments, R¹ is NHCO₂Q⁸, where Q⁸ is C₁₋₃ alkyl. In some of these embodiments, R¹ is NHCO₂CH₃. In other of these embodiments, R¹ is NHCO₂C₂H₅. In other of these embodiments, R¹ is NHCO₂C(CH₃)₂.

In some embodiments, R¹ is OCONR^{N5}R^{N6}. In some of these embodiments, R^{N5} and R^{N6} together with the N atom to which they are bound form a C₄ N-containing heterocyclyl group. In other of these embodiments, R^{N5} and R^{N6} are both Me.

R⁴

In some embodiments, R⁴ is H.

In some embodiments, R⁴ is F.

In some embodiments, R⁴ is methyl.

R¹ and R⁴

When R¹ and R⁴ together with the carbon atom to which they are bound may form a C₄₋₆ cycloalkyl, they may form cylcobutyl, cylcopentyl or cylcohexyl.

In some of these embodiments, R¹ and R⁴ together with the carbon atom to which they are bound form cylcobutyl.

In some of these embodiments, R¹ and R⁴ together with the carbon atom to which they are bound form cylcopentyl.

In some of these embodiments, R¹ and R⁴ together with the carbon atom to which they are bound form cylcohexyl.

Cy

In some embodiments, Cy is pyridyl.

In some embodiments, Cy is oxazolyl.

In some embodiments, Cy is cyclohexyl.

In some embodiments, Cy is unsubstituted phenyl.

In some embodiments, Cy is phenyl bearing a single substituent. The substituent may be in the 2-, 3- or 4-position. In some of these embodiments, the substituent is in the 2-position. In other of these embodiments, the substituent is in the 3-position. In other of these embodiments, the substituent is in the 4-position.

In some embodiments, the phenyl substituent is R². In some of these embodiments, R² is CH₃ (methyl). In other of these embodiments, R² is CH₂F. In other of these embodiments, R² is CHF₂. In other of these embodiments, R² is CF₃.

In certain embodiments, R² may be CF₃.

In some embodiments, the phenyl substituent is OR⁵. In some of these embodiments, R⁵ is H. In other of these embodiments, R⁵ is CH₃ (methyl). In other of these embodiments, R⁵ is CH₂F. In other of these embodiments, R⁵ is CHF₂. In other of these embodiments, R⁵ is CF₃. In other of these embodiments, R⁵ is cyclopropyl.

In some embodiments, the phenyl substituent is benzyloxy.

In some embodiments, the phenyl substituent is halo. In some of these embodiments, the halo group is F. In others of these embodiments the halo group is Cl.

In some embodiments, the phenyl substituent is cyano.

In some embodiments, the phenyl substituent is amino (NH₂).

In some embodiments, the phenyl substituent is C₅ heteroaryl, optionally substituted by methyl, CH₂OH, CH₂OCH₃ or ═O. In some of these embodiments, Cy is unsubstituted C₅ heteroaryl. In others of these embodiments, Cy is C₅ heteroaryl substituted with methyl; In others of these embodiments, Cy is C₅ heteroaryl substituted with CH₂OH. In others of these embodiments, Cy is C₅ heteroaryl substituted with CH₂OCH₃. In others of these embodiments, Cy is C₅ heteroaryl substituted with ═O.

In some of these embodiments, the C₅ heteroaryl group may contain at least one nitrogen ring atom. In these embodiments, any other ring heteroatoms may be selected from nitrogen and oxygen. In certain embodiments, the C₅ heteroaryl group may be selected from pyrrolyl, oxazolyl, isoxazolyl, oxadiazolyl, imidazolyl, pyrazolyl and triazolyl. In other certain embodiments, the C₅ heteroaryl group may be selected from oxazolyl, pyrazolyl and triazolyl.

In some embodiments, the phenyl substituent is phenyl, i.e. Cy is biphenyl.

In some embodiments, the phenyl substituent is pyridyl, optionally substituted with methyl. In some of these embodiments, the phenyl substituent is unsubstituted pyridyl. In others of these embodiment, the phenyl substituent is pyridyl substituted by methyl.

In some embodiments, the phenyl substituent is COQ⁵, where Q⁵ is selected from OH, OCH₃ and NR^{N1}R^{N2}.

In some embodiments, Q⁵ is OH.

In other embodiments, Q⁵ is OCH₃.

In other embodiments, Q⁵ is NR^{N1}R^{N2}. In some of these embodiments, R^{N1} and R^{N2} are both H. In other of these embodiments, R^{N1} and R^{N2} are both Me. In other of these embodiments, R^{N1} is H and R^{N2} is Me.

In some embodiments, the phenyl substituent is CH₂OQ⁶, where Q⁶ is H or Me. In some of these embodiments, the phenyl substituent is CH₂OH. In other of these embodiments, the phenyl substituent is CH₂OMe.

As discussed above, the compounds of the present invention have a stereochemical centre at the carbon atom to which R¹ and Cy are bound when R¹ is not H and R¹ and Cy are different. In some embodiments, these compounds are racemic. In other embodiments, these compounds are in enantiomeric excess. In other embodiments, these compounds are substantially enantiomerically pure/exist as a single enantiomer.

R¹ and Cy

In some embodiments, R¹ is H and Cy has a substituent in the 2-position, selected from OCHF₂ and a C₅ heteroaryl group selected from oxazolyl, pyrazolyl and triazolyl.

In some embodiments, R¹ is selected from oxazolyl, methyl-oxadiazolyl and pyrazolyl and Cy bears no substituent in the 2-position, i.e. Cy may be unsubstituted or bear a substituent in the 3- or 4-positions.

Compounds of particular interest include those of the examples.

In certain embodiments, the compounds of the invention are of formula Ia:

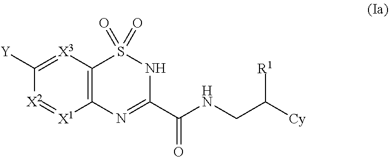

wherein:

X¹, X² and X³ are each selected from CH and N, where none or one of X¹, X² and X³ are N; Y is selected from the group consisting of: H; halo; cyano; R², where R² is selected from CH₃, CH₂F, CHF₂ and CF₃; ethynyl; cyclopropyl; OR³, where R³ is selected from H, CH₃, CH₂F, CHF₂ and CF₃; NR^{N1}R^{N2}, where R^{N1} and R^{N2} are independently selected from H and CH₃; COQ¹, where Q¹ is selected from C₁₋₄ alkyl, OH, OC₁₋₄ alkyl and NR^{N1}R^{N2}; NHSO₂Q³, where Q³ is C₁₋₃ alkyl; pyridyl; C₅ heteroaryl, which may be substituted by a group selected from C₁₋₃ alkyl, which itself may be substituted by OH or CONR^{N1}R^{N2};

Cy is selected from pyridyl and optionally substituted phenyl, where the optional substituents are selected from the group consisting of: R²; O^{R3}; benzyloxy; halo; cyano; amino; C₅ heteroaryl, optionally substituted by methyl; pyridyl, optionally substituted with methyl; COQ⁵, where Q⁵ is selected from OH and NR^{N1}R^{N2}; and CH₂OQ⁶, where Q⁶ is H or Me;

R¹ is selected from the group consisting of: F; phenyl; pyridyl; C₅ heteroaryl, optionally substituted by methyl; C₉ heteroaryl; OH; OMe; OPh; COQ⁴, where Q⁴ is selected from OH, C₁₋₃ alkyloxy, NR^{N5}R^{N6}, where R^{N5} is selected from H and Me, and R^{N5} is selected from C₁₋₄ alkyl, which itself may be substituted by CONHMe, or where R^{N5} and R^{N6} together with the N atom to which they are bound form a C₄₋₆ N-containing heterocyclyl group; (CH₂)ₙOH, where n is 1 or 2; NHCO₂Q⁴, where Q⁴ is C₁₋₃ alkyl; OCONR^{N5}R^{N6}; and

when Cy is pyridyl or substituted phenyl, R¹ may additionally be selected from H.

### EXAMPLES

The following examples are provided solely to illustrate the present invention and are not intended to limit the scope of the invention, as described herein.

Acronyms

For convenience, many chemical moieties are represented using well known abbreviations, including but not limited to, methyl (Me), ethyl (Et), n-propyl (nPr), isopropyl (iPr), n-butyl (nBu), tert-butyl (tBu), phenyl (Ph), benzyl (Bn), methoxy (MeO), ethoxy (EtO), trimethylsilyl (TMS), tert-butyldimethylsilyl (TBDMS) and acetyl (Ac).

For convenience, many chemical compounds are represented using well known abbreviations, including but not limited to, methanol (MeOH), deuterated methanol (d₄-MeOD, methanol-d₄) ethanol (EtOH), isopropanol (i-PrOH), ether or diethyl ether (Et₂O), ethyl acetate (EtOAc), acetic acid (AcOH), acetonitrile (MeCN or ACN), dichloromethane (methylene chloride, DCM), trifluoroacetic acid (TFA), dimethylformamide (DMF), tetrahydrofuran (THF), dimethylsulfoxide (DMSO), deuterated chloroform (CDCl3, chloroform-d), diethylamine (DEA), deuterated dimethylsulfoxide (d₆-DMSO, DMSO-d₆), N-ethyl-N′-(3-dimethylaminopropyl)carbodiimide hydrochloride (EDCI.HCl, EDCI, EDCI.HCl), meta-chloroperoxybenzoic acid (mCPBA), 1,1′-bis(diphenylphosphino)ferrocene (dppf), tert-butyloxycarbonyl (Boc, BOC), 2-(trimethylsilyl)ethoxymethyl (SEM), triethylamine (Et₃N or TEA), 2-(1H-7-azabenzotriazol-1-yl)-1,1,3,3-tetramethyluronium hexafluorophosphate (HATU), 4-dimethylaminopyridine (DMAP), N,N-diisopropylethylamine (DIPEA or DIEA), 1,1′-bis(diphenylphosphino)ferrocene dichloropalladium (II) (PdCl₂(dppf)), trans-dichlorobis(triphenylphosphine)palladium(II) (PdCl₂(PPh₃)₂), tris(dibenzylideneacetone) dipalladium(0) (Pd₂(dba)₃), tetrakis(triphenylphosphine)palladium(0) (Pd(PPh₃)₄), propylphosphonic anhydride (T3P), hexamethylphosphoramide (HMPA), 1,2-dichloroethane (DCE), benzyl (Bn) and 1-hydroxybenzotriazole (HOBt), petroleum ether (pet. ether), 1,8-diazabicyclo[5.4.0]undec-7-ene (DBU), lithium bis(trimethylsilyl)amide (LHMDS or LiHMDS), acetylacetonate (acac), carbonyldiimidazole (CDI), methyl tert-butyl ether (MTBE), diisopropyl azodicarboxylate (DIAD), tetrabutylammonium fluoride (TBAF), methanesulfonyl chloride (MsCl).

In addition, TLC refers to thin layer chromatography.

Other abbreviations: overnight (o/n), retention time (rt, RT or Rₜ), minute(s) (min), hour(s) (h), room temperature (r.t., RT), concentrated (conc.), atmosphere (atm), aqueous (aq.), saturated (sat.), equivalent(s) (eq).

General Experimental Details

Unless otherwise stated the following generalisations apply. ¹H NMR spectra were recorded on a Bruker Ultrashield Plus (400 MHz) or a Bruker AVANCE (400 MHz). The multiplicity of a signal is designated by the following abbreviations: s, singlet; d, doublet; t, triplet; q, quartet; dd, doublet of doublets; dt, doublet of triplets; tt, triplet of triplets; br, broad; m, multiplet. All observed coupling constants, J, are reported in Hertz (Hz). Exchangeable protons are not always observed.

LCMS data was generated using either an Agilent 6100 Series Single Quad LCMS-A:, an Agilent 1260 Infinity Series UPLC/MS (LCMS-B) an Agilent 1200 Series Quad LCMS (LCMS-F) or Agilent 1200. Chlorine isotopes are reported as ³⁵Cl, Bromine isotopes are reported as either ⁷⁹Br or ⁸¹Br or both ⁷⁹Br/⁶¹Br.

LCMS Method A (LCMS-A):

Instrument: Agilent 6100 Series Single Quad LC/MS

Agilent 1200 Series HPLC

Pump: 1200 Series G1311A Quaternary pump

Autosampler: 1200 Series G1329A Thermostatted Autosampler

Detector: 1200 Series G1314B Variable Wavelength Detector

LC Conditions:

Reverse Phase HPLC analysis

Column: Luna C8 (2) 5 μm 50×4.6 mm 100 Å

Column temperature: 30° C.

Injection Volume: 5 μL

Solvent A: Water 0.1% Formic Acid

Solvent B: MeCN 0.1% Formic Acid

Gradient: 5-100% solvent B over 10 min

Detection: 254 nm or 214 nm

MS Conditions:

Ion Source: Quadrupole

Ion Mode: Multimode-ES

Drying gas temp: 300° C.

Vaporizer temperature: 200° C.

Capillary voltage (V): 2000 (positive)

Capillary voltage (V): 4000 (negative)

Scan Range: 100-1000

Step size: 0.1 sec

Acquisition time: 10 min

LCMS Method B (LCMS-B):

Instrument: Agilent 1260 Infinity Series UPLC/MS

Pump: 1260 Infinity G1312B Binary pump

Autosampler: 1260 Infinity G1367E 1260 HiP ALS

Detector: 1290 Infinity G4212A 1290 DAD

LC Conditions:

Reverse Phase HPLC analysis

Column: Poroshell 120 EC-C18 2.7 μm 50×3.0 mm

Column temperature: 35° C.

Injection Volume: 1 μL

Solvent A: Water 0.1% Formic Acid

Solvent B: MeCN 0.1% Formic Acid

Gradient: 5-100% solvent B over 3.8 min

Detection: monitored at 254 nm and 214 nm

MS Conditions:

Ion Source: Quadrupole

Ion Mode: API-ES

Drying gas temp: 350° C.

Capillary voltage (V): 3000 (positive)

Capillary voltage (V): 3000 (negative)

Scan Range: 100-1000

Step size: 0.1 sec

Acquisition time: 5 min

LCMS Method C (LCMS-C):

LC model: Agilent 1200

(Pump type: Binary Pump, Detector type: DAD)

MS model: Agilent G6110A Quadrupole

LC Conditions:

Column: Xbridge-C18, 2.5 μm, 2.1×30 mm

Column temperature: 30° C.

Acquisition of wavelength: 214 nm, 254 nm

Mobile phase: A: 0.07% HCOOH aqueous solution, B: MeOH

MS Conditions:

MS: Ion source: ES+ (or ES−) MS range: 50-900 m/z

Fragmentor: 60 Drying gas flow: 10 L/min

Nebulizer pressure: 35 psi Drying gas temperature: 350° C.

Vcap: 3.5 kV

Gradient Table:

| Flow (mL/min) | T (min) | A (%) | B (%) |
|---|---|---|---|
| 0.5 | 0.0 | 70 | 30 |
| 0.5 | 0.2 | 70 | 30 |
| 0.5 | 1.8 | 5 | 95 |
| 0.5 | 2.4 | 5 | 95 |
| 0.5 | 2.6 | 70 | 30 |
| 0.5 | 3.5 | 70 | 30 |

Sample Preparation:

The sample was dissolved in methanol, the concentration about 0.11-1 mg/mL, then filtered through syringe filter with 0.22 μm. (Injection volume: 1-10 μL)

LCMS Method D (LCMS-D):

LC model: Agilent 1200

(Pump type: Binary Pump, Detector type: DAD)

MS model: Agilent G6110A Quadrupole

LCMS Conditions:

LC: Column: Xbridge-C18, 2.5 μm, 2.1×30 mm

Column temperature: 30° C.

Acquisition of wavelength: 214 nm, 254 nm

Mobile phase: A: 0.07% HCOOH aqueous solution, B: MeOH

MS Conditions:

MS: Ion source: ES+ (or ES−) MS range: 50-900 m/z

Fragmentor: 60 Drying gas flow: 10 L/min

Nebulizer pressure: 35 psi Drying gas temperature: 350° C.

Vcap: 3.5 kV

Gradient Table:

| Flow | | | |
|---|---|---|---|
| (mL/min) | T (min) | A (%) | B (%) |
| 0.5 | 0.0 | 70 | 30 |
| 0.5 | 0.3 | 70 | 30 |
| 0.5 | 0.6 | 50 | 50 |
| 0.5 | 0.9 | 40 | 60 |
| 0.5 | 1.2 | 30 | 70 |
| 0.5 | 3.2 | 5 | 95 |
| 0.5 | 3.5 | 5 | 95 |
| 0.5 | 4.0 | 70 | 30 |
| 0.5 | 5.0 | 70 | 30 |

Sample Preparation:

The sample was dissolved in methanol, the concentration about 0.11-1 mg/mL, then filtered through the syringe filter with 0.22 μm. (Injection volume: 1-10 μL)

LCMS Method F (LCMS-F)

Instrument: Agilent 1200 series LC

Agilent 6120 Quadrupole Mass Detector

Agilent G1968D Active Splitter

LC Conditions:

Reverse Phase HPLC analysis

Column: Agilent Eclipse XDB-C18 5 μm 4.6×150 mm

Injection loop volume: 900 μL

QPump Solvent A: Water plus 0.1% formic acid

QPump Solvent B: Acetonitrile plus 0.1% formic acid

QPump Gradient: 5-100% B over 10 min

Flow rate: 1 mL/min

Detection: 254 nm

MS Conditions:

Ion Source: Quadrupole

Ion Mode: ES

Vaporiser Temp: 200° C.

Gas Temp: 300° C.

Capillary voltage positive (V): 4000

Capillary voltage negative (V): 4000

Scan Range: 100-700 Amu

Acquisition time: 10 min

Isocratic Pump (make-up flow):

Flow rate: 0.5 mL/min

Solvent: 50:50 water: acetonitrile plus 0.1% formic acid

LC-MS Method SYN-P-M (ES+)/SYN-N-M (ES−)

LC model: Agilent 1200; Pump type: Binary Pump, Detector type: DAD

MS model: Agilent G6110A Quadrupole

LC Conditions

LC: Column: Xbridge-C18, 2.5 μm, 2.1×30 mm

Column temperature: 30° C.
Acquisition of wavelength: 214 nm, 254 nm
Mobile phase: A: 0.07% HCOOH aqueous solution, B: MeOH
Run time: 5 min

MS Conditions

Ion source: ES+ (or ES−) MS range: 50-900 m/z

Fragmentor: 60 Drying gas flow: 10 L/min

Nebulizer pressure: 35 psi Drying gas temperature: 350° C.

Vcap: 3.5 kV

Gradient Table

| | Gradient | | | |
|---|---|---|---|---|
| | Flow | | A | B |
| Method Name (LCMS) | (ml/min) | T (min) | (% yield) | (% yield) |
| SYN-P-M (ES+) | 0.5 | 0.0 | 70 | 30 |
| or | 0.5 | 0.3 | 70 | 30 |
| SYN-N-M (ES−) | 0.5 | 0.6 | 50 | 50 |
| | 0.5 | 0.9 | 40 | 60 |
| | 0.5 | 1.2 | 30 | 70 |
| | 0.5 | 3.2 | 5 | 95 |
| | 0.5 | 3.5 | 5 | 95 |
| | 0.5 | 4.0 | 70 | 30 |
| | 0.5 | 5.0 | 70 | 30 |

Sample preparation: The sample was dissolved in methanol, approximate concentration 0.11˜1 mg/mL, then filtered through the syringes filter with 0.22 μm. (Injection volume: 1˜10 μL)

Preparative RP-HPLC:

Agilent 1260 Infinity HPLC system

UV detection at 210 nm and 254 nm

Gradient or isocratic elution through a Phenomenex Luna C8 (2) column 100 Å Axia (250×21.2 mm; particle size 5 μm)

Flow rate: 10 mL/min

Gradients are as specified in the individual examples.

Analytical thin-layer chromatography was performed on Merck silica gel 60 F254 aluminium-backed plates which were visualised using fluorescence quenching under UV light or a basic KMnO₄ dip or Ninhydrin dip.

Preparative thin-layer chromatography (preparative TLC or prep. TLC) was performed using Tklst (China), grand grade: (HPTLC): 8±2 μm>80%; (TLC): 10-40 μm. Type: GF254. Compounds were visualised by UV (254 nm).

Flash chromatography was performed using a Biotage Isolera purification system using either Grace, SepaFlash® or RediSep® silica cartridges.

Column chromatography was performed using Tklst (China), grand grade, 100-200 meshes silica gel.

Microwave irradiation was achieved using a CEM Explorer SP Microwave Reactor.

Where necessary, anhydrous solvents were purchased from Sigma-Aldrich or dried using conventional methods.

Additional Cartridges used are as follows:

Phase Separator:

Manufacturer: Biotage

Product: ISOLUTE® Phase Separator (3 mL unless otherwise stated)

SCX and SCX-2 Cartridges:

Manufacturer: Biotage

Product: ISOLUTE® SCX 1 g, (6 mL SPE Column unless otherwise stated)

Manufacturer: Biotage

Product: ISOLUTE® SCX-2 1 g (6 mL Column)

Manufacturer: Silicycle

Product: SCX-2 500 mg or 5 g or 10 g

Manufacturer: Agilent

Product: Bond Elut® SCX 10 g

Sample Extraction Cartridge:

Manufacturer: Waters Product: Oasis® HLB 35 cc (6 g) LP extraction cartridge

Si-Amine Cartridges:

Manufacturer: Agilent

Product: Bond Elut NH2 10 g

Synthesis of Intermediates

### (i) Ethyl 2H-benzo[e][1,2,4]thiadiazine-3-carboxylate 1,1-dioxide (I2)

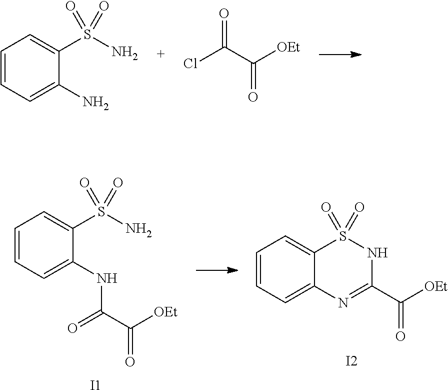

### a) Ethyl 2-oxo-2-((2-sulfamoylphenyl)amino)acetate (I1)

To solution of 2-aminobenzenesulfonamide (10.000 g, 58.070 mmol) in THF (500 mL), at 0° C., was added NEt₃ (8.50 mL, 60.973 mmol) followed by the dropwise addition of ethyl chlorooxoacetate (6.81 mL, 60.973 mmol) over 10 min. This was allowed to slowly warm to ambient temperature o/n. The precipitate was removed by filtration and the filtrate was concentrated in vacuo. The resulting solid was slurried in warm EtOAc (50 mL), then filtered. The solid material was washed with a further portion of EtOAc (50 mL), then air dried to reveal ethyl 2-oxo-2-((2-sulfamoylphenyl)amino)acetate (12.399 g, 78% yield) as a white solid. ¹H NMR (400 MHz, DMSO): δ 10.77 (s, 1H), 8.25 (dd, J=8.3, 1.1 Hz, 1H), 7.89 (dd, J=8.0, 1.5 Hz, 1H), 7.69 (s, 2H), 7.69-7.64 (m, 1H), 7.37 (ddd, J=8.0, 7.4, 1.2 Hz, 1H), 4.32 (q, J=7.1, 7.1, 7.1 Hz, 2H), 1.33 (t, J=7.1, 7.1 Hz, 3H). LC-MS (LCMS:B): rt 3.409 min; m/z 271.1 [M−H] (−ve); no corresponding product ions present in +ve mode.

### b) Ethyl 2H-benzo[e][1,2,4]thiadiazine-3-carboxylate 1,1-dioxide (I2)

To dry EtOH (200 mL), under a nitrogen atmosphere, was added NaH (60% dispersion in mineral oil, 1.463 g, 36.580 mmol) cautiously. This was allowed to stir for 15 min, upon which ethyl 2-oxo-2-(2-sulfamoylphenylamino)acetate (11) (8.300 g, 30.483 mol) was added. This stirred for a further 3 h, upon which water (400 mL) was added and the pH adjusted to 3 using 2N aqueous HCl. The EtOH was removed in vacuo, and the precipitate filtered. The solid was washed with water, then air dried to reveal ethyl 2H-benzo[e][1,2,4]thiadiazine-3-carboxylate 1,1-dioxide (12) (5.575 g, 72% yield) as a white solid. ¹H NMR (400 MHz, DMSO): δ 12.74 (s, 1H), 7.88-7.85 (m, 1H), 7.79-7.72 (m, 2H), 7.54 (ddd, J=8.2, 6.3, 2.1 Hz, 1H), 4.40 (q, J=7.1, 7.1, 7.1 Hz, 2H), 1.36 (t, J=7.1, 7.1 Hz, 3H). LC-MS (LCMS:B): rt 3.349 min; m/z 255.1 [M+H]⁺.

### (ii) Ethyl 7-bromo-2H-benzo[e][1,2,4]thiadiazine-3-carboxylate 1,1-dioxide (I5)

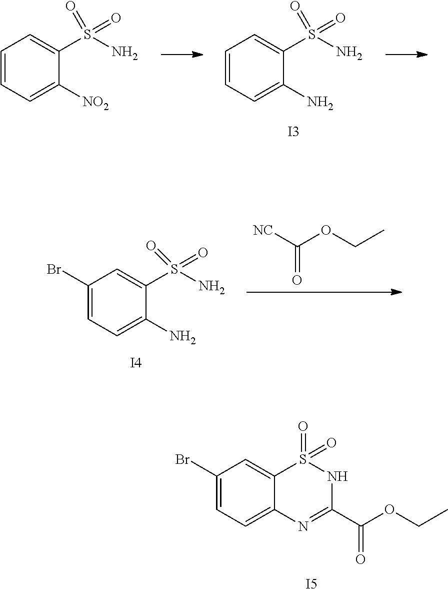

### a) 2-Aminobenzenesulfonamide (I3)

A mixture of 2-nitrobenzenesulfonamide (50 g, 245 mmol), zinc dust (81 g, 1.24 mol) and NH₄Cl (66 g, 1.24 mol) in EtOH (750 mL) and water (200 mL) was heated at 80° C. overnight then allowed to cool to r.t. The mixture was filtered and the solid was washed with DCM (20 mL). The filtrate was washed with brine, dried over sodium sulfate, filtered and concentrated to give the product (35 g, 82% yield) as a yellow solid. LCMS (ES-API): Rₜ 0.38 min; m/z 173.1 [M+H]⁺.

### b) 2-Amino-5-bromobenzenesulfonamide (I4)

To a solution of 2-aminobenzenesulfonamide (I3) (20 g, 116 mmol) in CH₃COOH (200 mL) at r.t. was added a solution of Br₂ (10.9 g, 68 mmol) in CH₃COOH (200 mL) and the mixture was stirred at r.t. for 20 min then poured into ice-water (400 mL). The mixture was filtered and the solid was washed with water (100 mL). The combined filtrates were concentrated to give the product as a brown solid (17.2 g, 59% yield). LCMS (ES-API): Rₜ 1.11 min; m/z 250.9/252.9 [M+H]⁺.

### c) Ethyl 7-bromo-2H-benzo[e][1,2,4]thiadiazine-3-carboxylate 1,1-dioxide (I5)

To a solution of 2-amino-5-bromobenzenesulfonamide (I4) (10 g, 39.8 mmol) and ethyl carbonocyanidate (39.5 g, 398 mmol) in CH₃COOH (100 mL) at r.t. was added conc. HCl (10 mL) and the mixture was heated at 80° C. for 3 h then poured into ice-water (200 mL) and stirred for 1 h. The mixture was filtered and the solid was washed with water (100 mL). The combined filtrates were concentrated to give the product as a white solid (8 g, 60% yield). LCMS (ES-API): Rₜ 1.78 min; m/z 332.9/334.9 [M+H]⁺.

### (iii) Ethyl 7-iodo-2H-benzo[e][1,2,4]thiadiazine-3-carboxylate 1,1-dioxide (I7)

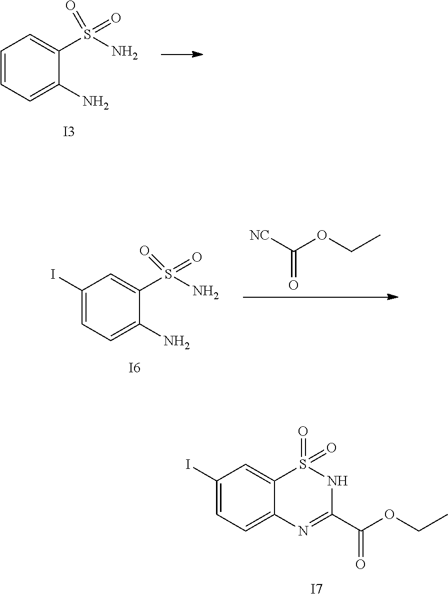

### a) 2-Amino-5-iodobenzenesulfonamide (I6)

To a solution of 2-aminobenzenesulfonamide (I3) (3 g, 17.4 mmol) in CHCl₃ (150 mL) at −20° C. was added a solution of ICI (1.98 g, 12.2 mmol) in CHCl₃ (150 mL) and the mixture was stirred at −20° C. for 30 min. The mixture was filtered and the solid was washed with CHCl₃ (50 mL) and 2 M aqueous NaHCO₃ (50 mL) then dried to give the product as a brown solid (3.3 g, 63% yield). LCMS (ES-API) Rₜ 1.34 min; m/z 298.9 [M+H]⁺.

### b) Ethyl 7-iodo-2H-benzo[e][1,2,4]thiadiazine-3-carboxylate 1,1-dioxide (I7)

To a solution of 2-amino-5-iodobenzenesulfonamide (I6) (2 g, 6.7 mmol) and ethyl carbonocyanidate (6.5 g, 67 mmol) in CH₃COOH (40 mL) at r.t. was added conc. HCl (2 mL) and the mixture was heated at 80° C. for 3 h then poured into ice-water (50 mL). The mixture was stirred for 1 h, filtered and the solid was washed with water (50 mL) then air dried to give the product as a brown solid (1.9 g, 75% yield). LCMS (ES-API) Rₜ 2.26 min; m/z 380.9 [M+H]⁺. ¹H NMR (400 MHz, d₆-DMSO) δ 12.8 (brs, 1H), 8.12 (d, J=2.0 Hz, 1H), 8.08 (dd, J=8.8, 2.0 Hz, 1H), 7.57 (d, J=8.8 Hz, 1H), 4.40 (t, J=7.2 Hz, 2H), 1.36 (t, J=7.2 Hz, 3H).

### (iv) Ethyl 2H-pyrido[3,4-e][1,2,4]thiadiazine-3-carboxylate 1,1-dioxide (I13)

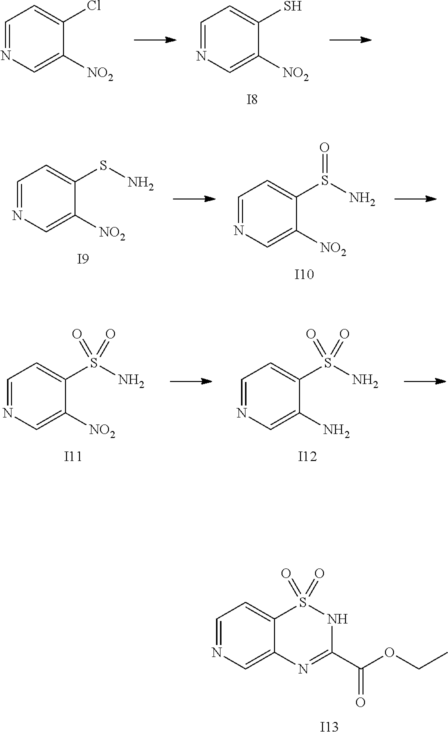

### a) 3-Nitropyridine-4-thiol (I8)

A mixture of 4-chloro-3-nitropyridine (15 g, 94.6 mmol) and NaSH.H₂O (14 g, 189 mmol) in MeOH (100 mL) was stirred at r.t. for 10 min then heated at 60° C. for 10 min. The solvent was removed and the residue was dissolved in water and acidified to pH 6 with 1 M aqueous HCl. The resulting precipitate was collected by filtration, washed with water and air dried to give the product (10 g, 69% yield) as a yellow solid. LCMS (ES-API): Rₜ 0.31 min; m/z 43.0 [M+H]⁺.

### b) S-(3-Nitropyridin-4-yl)thiohydroxylamine (I9)

To a 28% solution of aqueous NaClO (300 mL) at −10° C. was added conc. NH₄OH (60 mL) dropwise with stirring. After 20 min, a solution of 3-nitropyridine-4-thiol (I8) (17 g, 0.11 mol) in 2 M aqueous NaOH (60 mL) was added and stirring was continued for a further 1 h. The precipitate was collected by filtration and air dried to give the product (12 g, 67% yield) as a yellow solid. LCMS (ES-API): Rₜ 0.57 min; m/z 172.0 [M+H]⁺.

### c) 3-Nitropyridine-4-sulfinamide (I10)

To a mixture of S-(3-nitropyridin-4-yl)thiohydroxylamine (I9) (9.0 g, 52.6 mmol) in DCM (200 mL) at −5° C. was added m-CPBA (17 g, 78.9 mmol) in portions and the mixture was stirred at r.t. for 3 h. The mixture was concentrated and the residue was purified by column chromatography (EtOAc/Pet. Ether=1:1) to give the product (2.5 g, 25% yield) as a yellow solid. LCMS (ES-API): Rₜ 0.35 min; m/z 187.9 [M+H]⁺.

### d) 3-Nitropyridine-4-sulfonamide (I11)

To a suspension of 3-nitropyridine-4-sulfinamide (I10) (2.0 g, 10.68 mmol) and water (1.92 g, 107 mmol) in ACN (60 mL) at 0° C. was added iodosylbenzene (2.59 g, 11.75 mmol) and the mixture was allowed to warm to r.t. and stirred for 2 h. The mixture was concentrated and the residue was purified by column chromatography (MeOH/DCM=1:80) to give the product (1.75 g, 81% yield) as a yellow solid. LCMS (ES-API): Rₜ 0.36 min; m/z 203.9 [M+H]⁺.

### e) 3-Aminopyridine-4-sulfonamide (I12)

A mixture of 3-nitropyridine-4-sulfonamide (I11) (2.0 g, 9.89 mmol) and 10% Pd/C (200 mg) in EtOH (60 mL) was heated at 50° C. under 1 atm of H₂ for 16 h. The mixture was filtered through Celite® and the filtrate was concentrated to give the product (1.2 g, 70% yield) as a white solid. LCMS (ES-API): Rₜ 0.30; m/z 174.0 [M+H]⁺.

### f) Ethyl 2H-pyrido[3,4-e][1,2,4]thiadiazine-3-carboxylate 1,1-dioxide (I13)

A mixture of 3-aminopyridine-4-sulfonamide (I12) (500 mg, 2.89 mmol), ethyl 2-ethoxy-2-iminoacetate (629 mg, 4.34 mmol) and DBU (879 mg, 5.78 mmol) in EtOH (10 mL) was heated in a microwave at 135° C. for 30 min then allowed to cool to r.t. The mixture was concentrated and the residue was dissolved in water, acidified to pH 2 with 1 M aqueous HCl and extracted with EtOAc. The organic layer was washed with water and brine, dried over Na₂SO₄, filtered, concentrated and the residue was purified by preparative TLC (MeOH/DCM=1:20) to give the product (50 mg, 7% yield) as a yellow solid. LCMS (ES-API): Rₜ 0.51 min; m/z 255.9 [M+H]⁺. ¹H NMR (400 MHz, d₆-DMSO) δ 13.2 (brs, 1H), 9.09 (s, 1H), 8.81 (d, J=5.2 Hz, 1H), 7.88 (d, J=5.2 Hz, 1H), 4.42 (t, J=7.2 Hz, 2H), 1.37 (t, J=7.2 Hz, 3H).

### (v) Ethyl 2H-pyrido[4,3-e][1,2,4]thiadiazine-3-carboxylate 1,1-dioxide (I14)

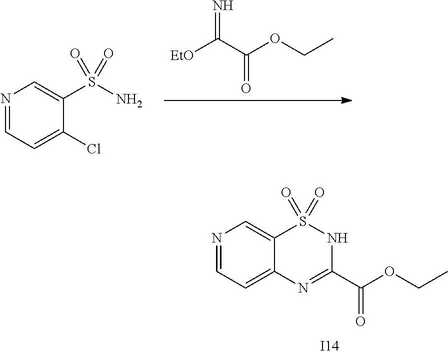

A mixture of 4-chloropyridine-3-sulfonamide (500 mg, 2.6 mmol), ethyl 2-ethoxy-2-iminoacetate (565 mg, 3.9 mmol) and DBU (790 mg, 5.2 mmol) in ethanol (10 mL) was heated in a sealed tube at 150° C. for 0.5 h then cooled to r.t. The mixture was diluted with water (5 mL), adjusted to pH 5 with 1 M aqueous HCl and exacted with DCM (10 mL×3). The combined organic extracts were washed with brine, dried over sodium sulfate and concentrated. The residue was purified by preparative TLC (MeOH/DCM=1:20, v/v) to give the product as a yellow solid (100 mg, 15% yield). LCMS (ES-API) Rₜ 0.47 min; m/z 256 [M+H]⁺. ¹H NMR (400 MHz, d₆-DMSO), 9.05 (s, 1H), 8.76 (d, J=5.6 Hz, 1H), 7.64 (d, J=5.6 Hz, 1H), 4.40 (q, J=7.2 Hz, 2H), 1.37 (t, J=7.2 Hz, 3H).

### (vi) Ethyl 2H-pyrido[2,3-e][1,2,4]thiadiazine-3-carboxylate 1,1-dioxide (I16)

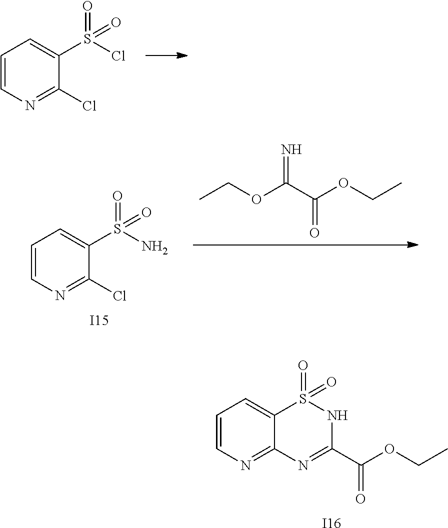

### a) 2-Chloropyridine-3-sulfonamide (I15)

A solution of 2-chloropyridine-3-sulfonyl chloride (3 g, 14.1 mmol) in dioxane (50 mL) was added to a solution of conc. NH₄OH (50 mL) at 0° C. and the mixture was stirred at r.t. for 2 h then extracted with DCM (3×10 mL). The combined organic extracts were washed with brine, dried over sodium sulfate, filtered and concentrated. The residue was purified by column chromatography (MeOH/CHCl₃=0:100-1:10) to give the product as a yellow solid (2.4 g, 88% yield). LCMS (ES-API): Rₜ 1.79 min; m/z 193/195 [M+H]⁺.

### b) Ethyl 2H-pyrido[2,3-e][1,2,4]thiadiazine-3-carboxylate 1,1-dioxide (I16)

A mixture of 2-chloropyridine-3-sulfonamide (I15) (50 mg, 0.26 mmol), ethyl 2-ethoxy-2-iminoacetate (56 mg, 0.39 mmol) and DBU (79 mg, 0.52 mmol) in ethanol (5 mL) was heated in a sealed tube at 130° C. for 0.5 h then cooled to r.t. The mixture was diluted with water (5 mL), adjusted to pH 5 with 1 M aqueous HCl and extracted with DCM (10 mL×3).

The combined organic extracts were washed with brine, dried over sodium sulfate, filtered and concentrated. The residue was purified by preparative TLC (MeOH/DCM=1:20) to give the product as a yellow solid (10 mg, 15% yield). LCMS (ES-API) Rₜ 0.51 min; m/z 256.1 [M+H]⁺. ¹H NMR (400 MHz, d₆-DMSO) 8.81 (dd, J=4.8, 2.0 Hz, 1H), 8.43 (dd, J=8.0, 1.6 Hz, 1H), 7.63 (dd, J=8.0, 4.8 Hz, 1H), 4.41 (q, J=7.2 Hz, 2H), 1.37 (t, J=7.2 Hz, 3H).

### (vii) Ethyl 2H-pyrido[3,2-e][1,2,4]thiadiazine-3-carboxylate 1,1-dioxide (I21)

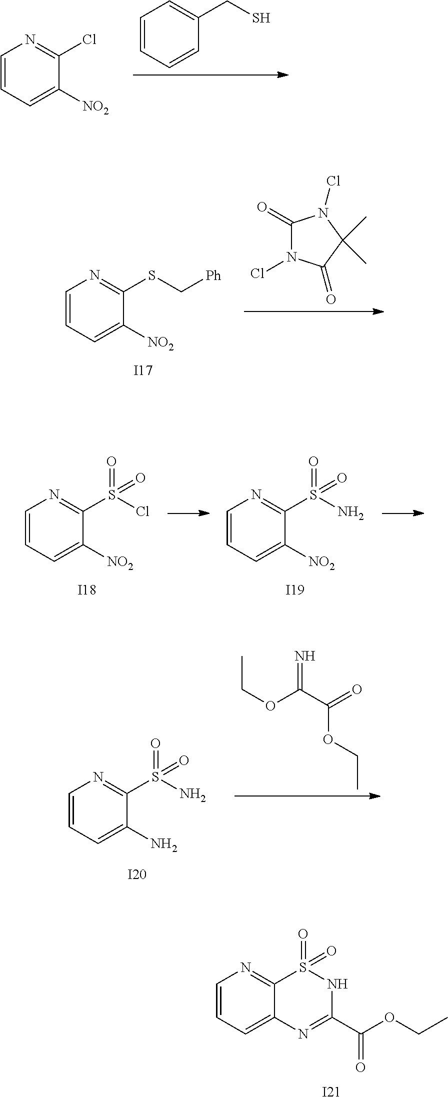

### a) 2-(Benzylthio)-3-nitropyridine (I17)

A mixture of 2-chloro-3-nitropyridine (10 g, 63.1 mmol), phenylmethanethiol (8.6 g, 69.4 mmol) and K₂CO₃ (9.6 g, 69.4 mmol) in EtOH (300 mL) and water (60 mL) was stirred at r.t. overnight. Water was added with stirring and the resulting precipitate was collected by filtration, washed with water and dried under reduced pressure to give the product (10 g, 65% yield) as a yellow solid. LCMS (ES-API): Rₜ 2.96 min; m/z 247.0 [M+H]⁺.

### b) 3-Nitropyridine-2-sulfonyl Chloride (I18)

To a mixture of 2-(benzylthio)-3-nitropyridine (I17) (6 g, 24.4 mmol) in water (24 mL), AcOH (12 mL) and DCM (84 mL) at r.t. was added 1,3-dichloro-5,5-dimethylimidazolidine-2,4-dione (14.4 g, 73.1 mmol). The mixture was stirred at r.t. for 16 h then poured into water and extracted with DCM. The organic extract was washed with water, brine, dried over Na₂SO₄, filtered and concentrated to give the product (5 g), which was used directly in the next step without further purification.

### c) 3-Nitropyridine-2-sulfonamide (I19)

A solution of 3-nitropyridine-2-sulfonyl chloride (I18) (5 g, 22.5 mmol) in DCM (100 mL) was added dropwise to a solution of conc. NH₄OH (100 mL) at 0° C. with stirring. The mixture was stirred for 30 min then concentrated and the residue was purified by column chromatography (MeOH/DCM=1:30) to give the product (2.2 g, 44% for two steps) as a yellow solid. LCMS (ES-API): Rₜ 0.43 min; m/z 204.0 [M+H]⁺.

### d) 3-Aminopyridine-2-sulfonamide (I20)

A mixture of 3-nitropyridine-2-sulfonamide (I19) (1.0 g, 4.92 mmol) and 10% Pd/C (100 mg) in EtOH (20 mL) was heated at 50° C. under 1 atm of H₂ for 16 h. The mixture was filtered through Celite and the filtrate was concentrated to give the product (0.7 g, 82% yield) as a yellow solid. LCMS (ES-API): Rₜ 0.28 min; m/z 714.0 [M+H]⁺.

### e) Ethyl 2H-pyrido[3,2-e][1,2,4]thiadiazine-3-carboxylate 1,1-dioxide (I21)

A mixture of 3-aminopyridine-2-sulfonamide (I20) (500 mg, 2.89 mmol), ethyl 2-ethoxy-2-iminoacetate (629 mg, 4.34 mmol) and DBU (879 mg, 5.78 mmol) in EtOH (10 mL) was heated at 125° C. in a microwave for 25 min then cooled to r.t. The mixture was concentrated and the residue was diluted with water, acidified to pH 2 with 1 M aqueous HCl and extracted with EtOAc. The organic layer was washed with water, brine, dried over Na₂SO₄, filtered and concentrated. The residue was purified by prep. TLC (MeOH/DCM=1:20) to give the desired product (120 mg, 16% yield) as a yellow solid. LCMS (ES-API): Rₜ 0.39 min; m/z 256.0 [M+H]⁺. ¹H NMR (400 MHz, d₆-DMSO) δ 12.8 (brs, 1H), 8.70 (dd, J=4.4 Hz, 1.2 Hz, 1H), 8.17 (dd, J=8.4 Hz, 1.2 Hz, 1H), 7.81 (dd, J=8.4, 4.8 Hz, 1H), 4.41 (q, J=7.2 Hz, 2H), 1.36 (t, J=7.1 Hz, 3H).

### (viii) Methyl 7-(trifluoromethyl)-2H-benzo[e][1,2,4]thiadiazine-3-carboxylate 1,1-dioxide (I24)

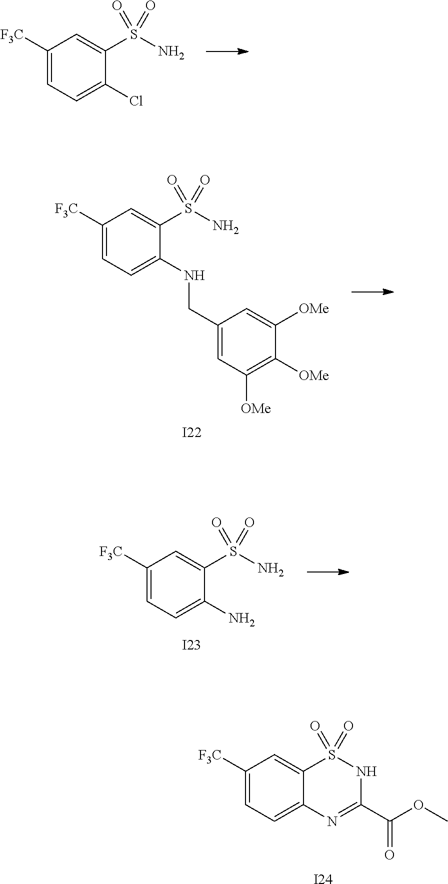

### a) 5-(Trifluoromethyl)-2-((3,4,5-trimethoxybenzyl)amino)benzenesulfonamide (I22)

2-Chloro-5-(trifluoromethyl)benzenesulfonamide (1.34 g, 5.16 mmol) and 3,4,5-trimethoxybenzylamine (4.0 mL, 23 mmol) were heated at 130° C. overnight. The mixture was cooled and added to water (200 mL) with the aid of DMF (2 mL). The mixture was adjusted to pH 5 with acetic acid and sonicated. The mixture was filtered, the collected solid washed with water (2×50 mL) and air dried. Chromatography (40 g silica cartridge, 0-100% ethyl acetate/hexanes) gave the product as a solid (1.52 g, 70% yield). LCMS-A rt 5.93 min; m/z (negative ion) 419.1 [M−H]. ¹H NMR (400 MHz, DMSO-d6) δ 7.88 (dd, J=2.2, 0.9 Hz, 1H), 7.68 (s, 2H), 7.61 (dd, J=8.9, 2.4 Hz, 1H), 6.92-6.84 (m, 2H), 6.74 (s, 2H), 4.47 (d, J=5.9 Hz, 2H), 3.73 (s, 6H), 3.62 (s, 3H).

### b) 2-Amino-5-(trifluoromethyl)benzenesulfonamide (I23)

5-(Trifluoromethyl)-2-((3,4,5-trimethoxybenzyl)amino)benzenesulfonamide (I22) (1.878 g, 4.27 mmol) was dissolved in TFA (10 mL) and stirred at room temperature overnight. The mixture was concentrated in vacuo, the residue diluted with water (30 mL) and adjusted to pH 13 with 20% w/v aqueous sodium hydroxide. The mixture was filtered, the gummy precipitate washed with water (50 mL), and the precipitate transferred to a flask with ethanol. The mixture was concentrated in vacuo. Chromatography (40 g silica cartridge, 0-100% ethyl acetate/hexanes) gave the product as a yellow solid (766 mg, 75% yield). LCMS-A rt 5.31 min; m/z (negative ion) 239.0 [M−H]. ¹H NMR (400 MHz, DMSO-d6) δ 7.83-7.78 (m, 1H), 7.56-7.50 (m, 1H), 7.45 (s, 2H), 6.93 (dd, J=8.7, 0.9 Hz, 1H), 6.49 (s, 2H).

### c) Methyl 7-(trifluoromethyl)-2H-benzo[e][1,2,4]thiadiazine-3-carboxylate 1,1-dioxide (I24)

Methyl 2,2,2-trimethoxyacetate (0.521 mL, 3.58 mmol), 2-amino-5-(trifluoromethyl)benzenesulfonamide (I23) (172 mg, 0.716 mmol), 4-methylbenzenesulfonic acid (0.025 g, 0.14 mmol) and methanol (0.5 mL) were heated in the microwave (120° C./30 min). The mixture was cooled to room temperature and filtered to give the product as a white solid (52 mg). Additional product was recovered by chromatography of the filtrate (0-60% ethyl acetate/hexanes) (55 mg). Total product 107 mg, 47% yield. LCMS-B rt 3.13 min; m/z (negative ion) 306.8 [M−H]. ¹H NMR (400 MHz, DMSO-d6) δ 8.21-8.19 (m, 1H), 8.12 (dd, J=8.9, 2.1 Hz, 1H), 7.96 (d, J=8.8 Hz, 1H), 3.95 (s, 3H). 19F NMR (376 MHz, DMSO-d6) δ −61.03.

### (ix) 2-(Oxazol-2-yl)-2-phenylethanamine (I27)

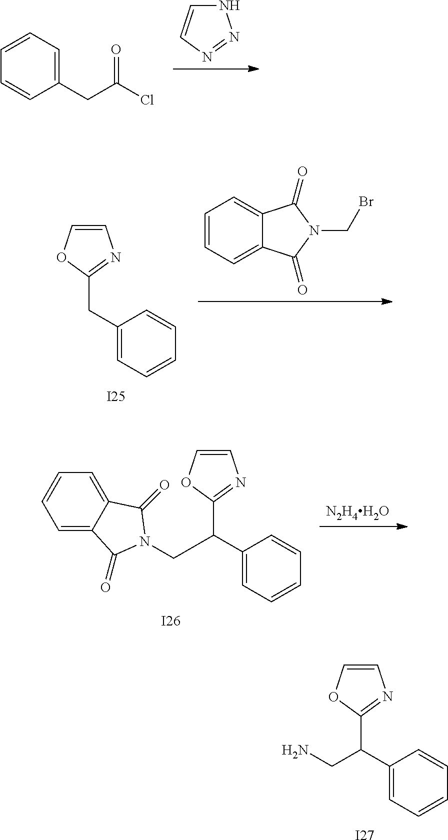

### a) 2-Benzyloxazole (I25)

To a solution of 1H-1,2,3-triazole (26.8 g, 388 mmol) in sulfolane (500 mL) at 0° C. was added 2-phenylacetyl chloride (50 g, 323 mmol) and K₂CO₃ (67 g, 485 mmol) and the mixture was stirred at r.t. for 20 min, then heated at 165° C. for 30 min. The mixture was cooled to r.t. and partitioned between water (3000 mL) and ether (500 mL). The layers were separated and the aqueous phase was extracted with ether (3×1000 mL). The combined organic extracts were washed with water, brine, dried over sodium sulfate, filtered and concentrated. The residue was purified by column chromatography (Petroleum ether/EtOAc=30:1-5:1) to give the desired product (25 g, 51% yield) as a yellow oil. LCMS (ES-API): Rₜ 2.78 min; m/z 160.1 [M+H]⁺.

### b) 2-(2-(Oxazol-2-yl)-2-phenylethyl)isoindoline-1,3-dione (I26)

To a solution of 2-benzyloxazole (I25) (10 g, 62.8 mmol) in THF (350 mL) at −78° C. under nitrogen was added LHMDS (1 M solution in THF, 75.4 mL, 75.4 mmol) dropwise. A solution of 2-(bromomethyl)isoindoline-1,3-dione (18.1 g, 75.4 mmol) in THF (50 mL) was then added dropwise and the mixture allowed to warm slowly to r.t. and stirred overnight. The mixture was diluted with a saturated aqueous NH₄Cl solution (300 mL) and water (150 mL), then extracted with DCM (1000 mL×3). The combined organic extracts were dried over anhydrous sodium sulphate, filtered, concentrated and purified by column chromatography (Petroleum ether/EtOAc=20:1-5:1) to give the desired product (5 g, 25% yield) as a white solid. LCMS (ES-API): Rₜ 2.62 min; m/z 319.1 [M+H]⁺.

### c) 2-(Oxazol-2-yl)-2-phenylethanamine (I27)

To a solution of 2-(2-(oxazol-2-yl)-2-phenylethyl)isoindoline-1,3-dione (I26) (4.2 g, 13.2 mmol) in ethanol (30 mL) was added hydrazine hydrate (2.7 g, 42.2 mmol) and the mixture was heated at 80° C. under nitrogen for 3 h. The mixture was filtered and the solid was washed with ethanol (30 mL). The filtrate was concentrated under reduced pressure and the residue was partitioned between DCM (50 mL) and saturated aqueous NaHCO₃ (50 mL). The layers were separated and the aqueous layer was extracted with DCM (100 mL×3). The combined organic extracts were washed with brine, dried over anhydrous sodium sulphate, filtered and concentrated to give the title product (1.4 g, 56% yield) as a yellow oil. ¹H NMR (400 MHz, d₆-DMSO) δ 7.99 (d, J=0.6 Hz, 1H), 7.34-7.30 (m, 2H), 7.27-7.20 (m, 3H), 7.17 (s, 1H), 4.18 (dd, J=8.3, 6.3 Hz, 1H), 3.24-3.23 (m, 1H), 3.03-2.98 (m, 1H). LCMS (ES-API): Rₜ 2.23 min; m/z 189.1 [M+H]⁺.

### (x) 2H-Benzo[e][1,2,4]thiadiazine-3-carbonyl chloride 1,1-dioxide (I30)

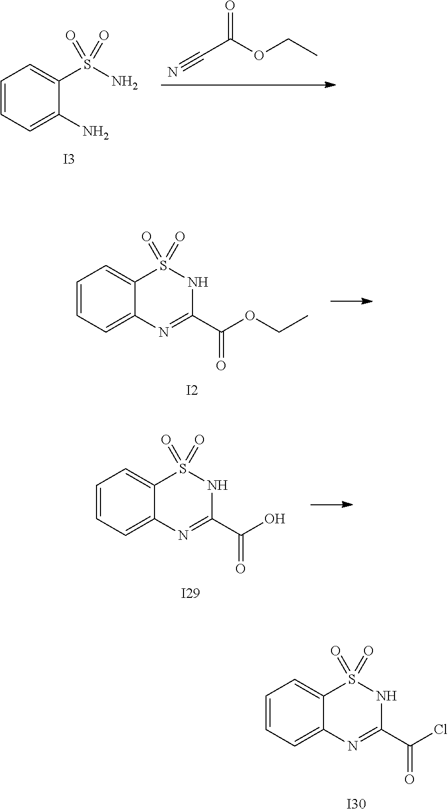

### a) Ethyl 2H-benzo[e][1,2,4]thiadiazine-3-carboxylate 1,1-dioxide (I2—Alternate Synthesis)

A mixture of 2-aminobenzenesulfonamide (I3) (17 g, 98.22 mmol) and ethyl cyanoacetate (16 g, 197.4 mmol) in acetic acid (150 mL) and conc. HCl (15 mL) was heated at 80° C. under N₂ for 3 h. Most of the solvent was removed and then water (300 mL) was added. The resulting mixture was stirred at 0° C. for 2 h and the resulting precipitate was collected by filtration and washed with water. The solid was dissolved in EtOAc, washed with water and dried over Na₂SO₄ The solvent was removed and the residue was purified by silica gel column chromatography (DCM/MeOH=100:1-40:1) to give the desired product (7.2 g, 29% yield) as a white solid. LCMS (ES-API): Rₜ 0.66 min; m/z 255.0 [M+H]⁺.

### b) 2H-Benzo[e][1,2,4]thiadiazine-3-carboxylic Acid 1,1-dioxide (I29)

A mixture of ethyl 2H-benzo[e][1,2,4]thiadiazine-3-carboxylate 1,1-dioxide (12) (10 g, 39.3 mmol) in 2 M aqueous LiOH (50 mL) was stirred at r.t. for 3 h. The mixture was diluted with water (100 mL) and washed with EtOAc (×2) then adjusted pH 1-2 and extracted with DCM (100 mL×2). The organic layers were combined, washed with water, brine and dried over Na₂SO₄. The solvent was removed to give the desired product (6 g, 67% yield) as a light yellow solid. LCMS (ES-API): Rₜ 0.34 min; m/z 227.0 [M+H]⁺.

### c) 2H-Benzo[e][1,2,4]thiadiazine-3-carbonyl chloride 1,1-dioxide (I30)

A mixture of 2H-benzo[e][1,2,4]thiadiazine-3-carboxylic acid 1,1-dioxide (I29) (2.5 g, 11.05 mmol) and SOCl₂ (20 mL) was heated at 85° C. for 2 h. The mixture was then concentrated to give the desired product which was used directly in the next step.

### (xi) 3-(1,1-Dioxido-2H-benzo[e][1,2,4]thiadiazine-3-carboxamido)-2-phenylpropanoyl chloride (I37)

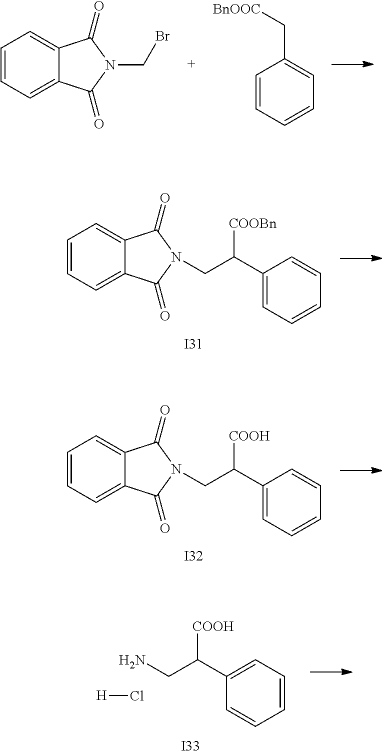

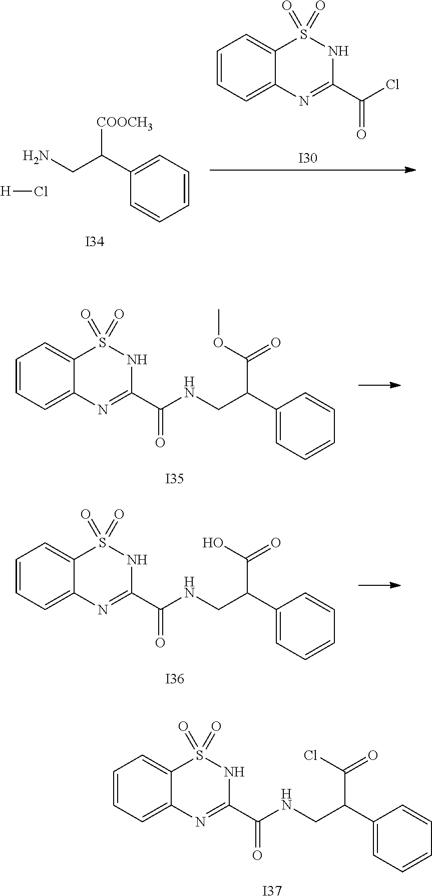

### a) Benzyl 3-(1,3-dioxoisoindolin-2-yl)-2-phenylpropanoate (I31)

To a solution of benzyl 2-phenylacetate (11.3 g, 50 mmol) in dry THF (100 mL) at −78° C. under nitrogen was added LiHMDS (2.5 M in THF, 40 mL, 100 mmol) dropwise over 25 min. A solution of 2-(bromomethyl)isoindoline-1,3-dione (14.4 g, 60 mmol) in THF (100 mL) was then added dropwise and the mixture was stirred at −78° C. for 2 h, then allowed to warm to r.t. and stirred overnight. The mixture was diluted with water (100 mL) and extracted with EtOAc (100 mL×3). The combined organic extracts were dried over sodium sulfate, filtered and concentrated. The residue was purified by column chromatography (DCM/MeOH=100:0-100:1) to give the desired product (12.5 g, 65% yield) as a white solid. LCMS (ES-API): Rₜ 2.78 min; m/z 386.1 [M+H]⁺.

### b) 3-(1,3-Dioxoisoindolin-2-yl)-2-phenylpropanoic Acid (I32)

A mixture of benzyl 3-(1,3-dioxoisoindolin-2-yl)-2-phenylpropanoate (I31) (8 g, 20.76 mmol) and 10% Pd/C (800 mg) in EtOAc (100 mL) and THF (100 mL) was heated at 45° C. under H₂ (1 atm) overnight. The mixture was filtered and the filtrate was concentrated to give the desired product (6 g, 98% yield) as a white solid. LCMS (ES-API): Rₜ 2.34 min; m/z 296.1 [M+H]⁺.

### c) 3-Amino-2-phenylpropanoic Acid Hydrochloride (I33)

To a solution of 3-(1,3-dioxoisoindolin-2-yl)-2-phenylpropanoic acid (I32) (6 g, 20.3 mmol) in ethanol (200 mL) was added hydrazine hydrate (1.93 g, 39.6 mmol) and the mixture was heated at 80° C. for 1 h. The solvent was removed, water (200 mL) was added and the mixture was again concentrated. The residue was diluted with water (200 mL) then adjusted to pH 2 with conc. HCl and stirred at r.t. for 30 min. The mixture filtered and the filtrate was concentrated to give the desired product (3.2 g, 95% yield) as a white solid. LCMS (ES-API): Rₜ 2.49 min; m/z 166.1 [M+H]⁺.

### d) Methyl 3-amino-2-phenylpropanoate Hydrochloride (I34)

Thionyl chloride (2 mL) was added dropwise to methanol (20 mL) at 0° C. followed by 3-amino-2-phenylpropanoic acid hydrochloride (I33) (1.6 g, 9.69 mmol) and the mixture was heated at reflux for 3 h. The solvent was removed and the residue was washed with EtOAc and dried to give the desired product (1.2 g, 57% yield) as a white solid, which was used directly in the next step.

### e) Methyl 3-(1,1-dioxido-2H-benzo[e][1,2,4]thiadiazine-3-carboxamido)-2-phenylpropanoate (I35; 112)

To a solution of methyl 3-amino-2-phenylpropanoate hydrochloride (I34) (400 mg, 2.23 mmol) in THF (30 mL) at 0° C. under N₂ was added NaHCO₃ (1.87 g, 22.3 mmol) and the mixture was stirred for 15 min. 2H-Benzo[e][1,2,4]thiadiazine-3-carbonyl chloride 1,1-dioxide (I30) (1.09 g, 4.46 mmol) was then added and stirring was continued at r.t. for 30 min. TEA (2.25 g, 223 mmol) was then added and the mixture was stirred for 10 min. Additional 2H-benzo[e][1,2,4]thiadiazine-3-carbonyl chloride 1,1-dioxide (I30) (1.09 g, 4.46 mmol) was added and stirring was continued at r.t. for 30 min. The mixture was partitioned between EtOAc (200 mL) and water (200 mL), the layers were separated and the organic phase was washed with water, 1 M aqueous HCl, brine, dried over sodium sulfate, filtered and concentrated. The residue was purified by prep. TLC (DCM/MeOH=50:1) to give the desired product (280 mg, 32% yield) as a light yellow solid. LCMS (ES-API): Rₜ 2.17 min; m/z 388.1 [M+H]⁺.

### f) 3-(1,1-Dioxido-2H-benzo[e][1,2,4]thiadiazine-3-carboxamido)-2-phenylpropanoic Acid (I36; 154)

To a solution of Methyl 3-(1,1-dioxido-2H-benzo[e][1,2,4]thiadiazine-3-carboxamido)-2-phenylpropanoate (135; 112) (560 mg, 1.445 mmol) in DCM (20 mL) was added 2 M aqueous NaOH (20 mL) and the mixture was stirred at r.t. for 2 h. The layers were separated and the aqueous layer was washed with DCM (50 mL) then adjusted to pH 2 with 2 M aqueous HCl. The resulting precipitate was collected by filtration and dried to give the desired product (230 mg, 43% yield) as a white solid. LCMS (ES-API): Rₜ 2.47 min; m/z 374.1 [M+H]⁺.

### g) 3-(1,1-Dioxido-2H-benzo[e][1,2,4]thiadiazine-3-carboxamido)-2-phenylpropanoyl Chloride (I37)

A solution of 3-(1,1-dioxido-2H-benzo[e][1,2,4]thiadiazine-3-carboxamido)-2-phenylpropanoic acid (136) (100 mg, 0.268 mmol) in thionyl chloride (10 mL) was heated at 90° C. for 3 h. The solvent was removed and the residue was used next step without further purification.

### (ix) 2-(Oxazol-2-yl)-2-phenylethanamine (I27)—Alternative Preparation

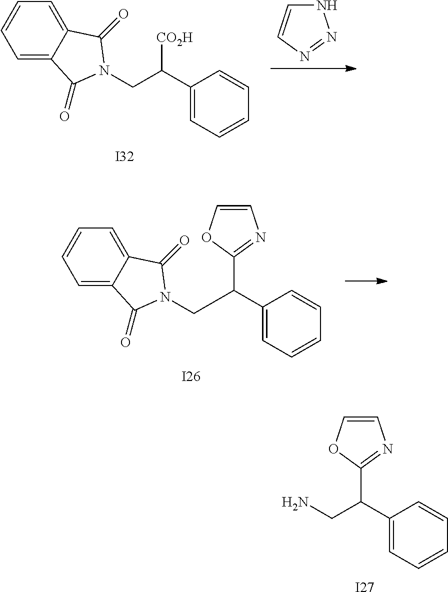

### a) 2-(2-(Oxazol-2-yl)-2-phenylethyl)isoindoline-1,3-dione (I26)

A mixture of 3-(1,3-dioxoisoindolin-2-yl)-2-phenylpropanoic acid (I32) (3.00 g, 10.2 mmol) and thionyl chloride (10 mL) was stirred at 80° C. under an atmosphere of nitrogen for 3 h. The mixture was cooled to r.t. and excess thionyl chloride was evaporated in vacuo. The solid residue was dissolved in sulfolane (10 mL) before 1H-1,2,3-triazole (0.83 mL, 14 mmol) and K₂CO₃ (2.81 g, 20.3 mmol) were added, and the mixture stirred at 150° C. under an atmosphere of nitrogen for 30 min. After returning to room temperature, water was added (40 mL) and the aqueous layer was extracted with EtOAc (3×50 mL). The combined organics were washed with brine, dried (MgSO₄), filtered and the solvent removed in vacuo. The crude solid was purified by column chromatography (Biotage Isolera, 80 g SiO₂ cartridge, 0-40% EtOAc in petroleum benzine 40-60° C.) to give the title compound as a white solid (5.37 g, ˜60% purity, quantitative yield assumed for next step); ¹H NMR (400 MHz, DMSO-d₆) δ 8.06-8.00 (m, 1H), 7.81 (s, 4H), 7.31-7.21 (m, 5H), 7.19-7.13 (m, 1H), 4.76-4.67 (m, 1H), 4.31-4.17 (m, 2H); LCMS-B: rt 3.30 min; m/z 319.1 [M+H]⁺.

### b) 2-(Oxazol-2-yl)-2-phenylethan-1-amine (I27)

Hydrazine hydrate (50-60%, 2.53 mL, ˜41 mmol) was added to a suspension of 2-(2-(oxazol-2-yl)-2-phenylethyl)isoindoline-1,3-dione (I26) (5.37 g, ˜60% purity, 10.1 mmol) in EtOH (100 mL). The mixture was stirred at 80° C. for 3.5 h, cooled to room temperature and the volatiles removed in vacuo. The solid was suspended in aq. HCl (2 M, ˜50 mL) and H₂O (˜50 mL) and the precipitate removed by filtration. The aqueous filtrate was washed with DCM (3×75 mL) and then brought to pH ˜14 with the addition of aq. NaOH (2 M).

The aqueous layer was extracted with DCM (3×75 mL), the organics combined, washed with brine, dried (MgSO₄), filtered and the solvent removed in vacuo to give the title compound as a colourless oil (0.951 g, 50% yield); ¹H NMR (400 MHz, DMSO-d₆) δ 8.04-7.94 (m, 1H), 7.35-7.29 (m, 2H), 7.26-7.20 (m, 3H), 7.19-7.16 (m, 1H), 4.18 (dd, J=8.4, 6.2 Hz, 1H), 3.24 (dd, J=12.8, 8.4 Hz, 1H), 3.08-2.94 (m, 1H), exchangeable NH₂ protons not observed; LCMS-B: rt 0.98 min; m/z 189.1 [M+H]⁺.

### (xii) N-(2-Amino-2-phenylethyl)-2H-benzo[e][1,2,4]thiadiazine-3-carboxamide 1,1-dioxide Hydrochloride (I41)

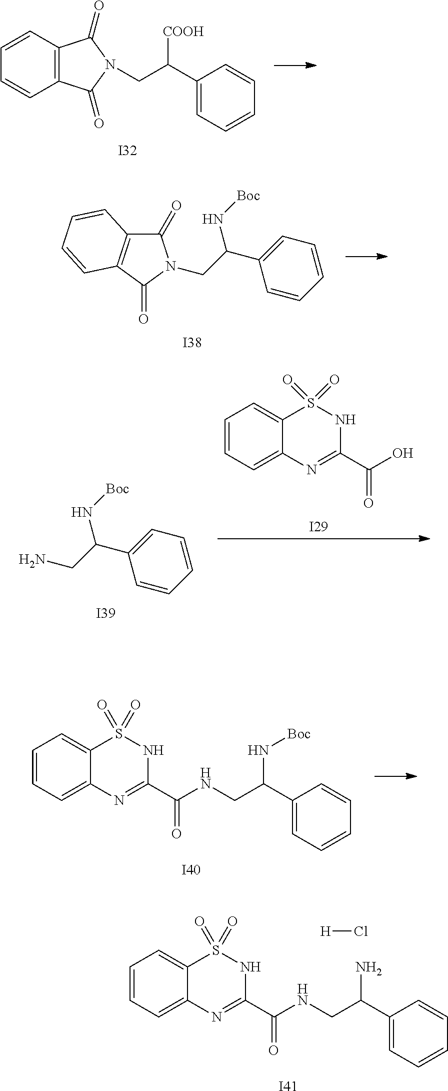

### a) tert-Butyl (2-(1,3-dioxoisoindolin-2-yl)-1-phenylethyl)carbamate (I38)

A mixture of 3-(1,3-dioxoisoindolin-2-yl)-2-phenylpropanoic acid (I32) (5 g, 16.9 mmol), DPPA (5.59 g, 20.3 mmol), Boc₂O (7.39 g, 33.9 mmol) and TEA (11.8 mL, 84.6 mmol) in t-BuOH (50 mL) and dioxane (80 mL) was heated at 100° C. overnight. The solvent was removed to give a residue which was purified by silica gel chromatography (Petroleum ether/EtOAc=100:1-3:1) to give the desired product (4.5 g, 73% yield) as a white solid. LCMS (ES-API): Rₜ 0.2.84 min; m/z 389.1 [M+Na]⁺.

### b) tert-Butyl (2-amino-1-phenylethyl)carbamate (I39)

To a solution of tert-butyl (2-(1,3-dioxoisoindolin-2-yl)-1-phenylethyl)carbamate (I38) (11 g, 30.0 mmol) in EtOH (400 mL) was added NH₄.H₂O (4 mL, 60.0 mmol) and the mixture was heated at 80° C. for 2 h under N₂ atmosphere. The mixture was filtered and the solid was washed with more ethanol (2 mL). The combined filtrates were concentrated and purified by chromatography (DCM/MeOH=50:1) to give the product (2.85 g, 40% yield) as a yellow oil. LCMS (ES-API): Rₜ 0.90 min; m/z 237.2 [M+H]⁺.

### c) tert-Butyl (2-(1,1-dioxido-2H-benzo[e][1,2,4]thiadiazine-3-carboxamido)-1-phenylethyl)carbamate (I40)

To a solution of tert-butyl (2-amino-1-phenylethyl)carbamate (I39) (2.85 g, 12.0 mmol), 2H-benzo[e][1,2,4]thiadiazine-3-carboxylic acid 1,1-dioxide (I29) (1.23 g, 5.0 mmol), EDCI (3.5 g, 18.1 mmol) and HOBT (2.45 g, 18.1 mmol) in DMF (50 mL) was added TEA (4.8 g, 48.2 mmol) and the mixture was stirred at r.t. overnight. The mixture was diluted with sat. aq. NaHCO₃ (30 mL) and extracted with DCM (3×50 mL). The combined organic extracts were washed with water (50 mL), brine (50 mL), dried over Na₂SO₄, filtered and concentrated. The residue was purified by column chromatography (DCM/MeOH=70:1) to give the product (0.73 g, 13% yield) as a yellow solid. LCMS (ES-API): Rₜ 2.54 min; m/z 445.1 [M+H]⁺.

### d) N-(2-Amino-2-phenylethyl)-2H-benzo[e][1,2,4]thiadiazine-3-carboxamide 1,1-dioxide Hydrochloride (I41)

To a mixture of tert-butyl (2-(1,1-dioxido-2H-benzo[e][1,2,4]thiadiazine-3-carboxamido)-1-phenylethyl)carbamate (I40) (600 mg, 1.35 mmol) in DCM (6 mL) was added 2 M HCl in EtOAc (18 mL) and the mixture was stirred at r.t. for 2 h. The mixture was concentrated to give the product (500 mg, 97% yield) as an off-white solid. LCMS (ES-API): Rₜ 0.60 min; m/z 345.1 [M+H]⁺.

### (xiii) N-(3-Amino-2-phenylpropyl)-2H-benzo[e][1,2,4]thiadiazine-3-carboxamide 1,1-dioxide Hydrochloride (I46)

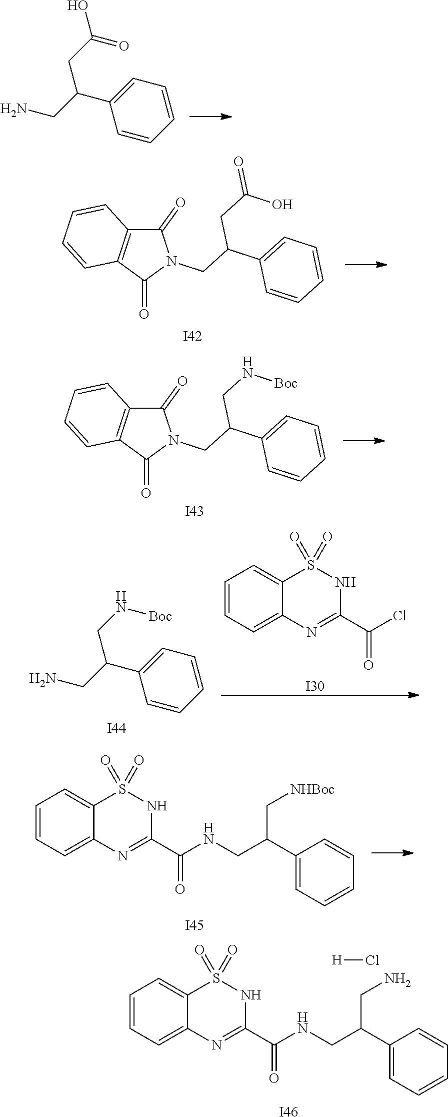

### a) 4-(1,3-Dioxoisoindolin-2-yl)-3-phenylbutanoic Acid (I42)

A solution of 4-amino-3-phenylbutanoic acid (2.6 g, 14.5 mmol) and phthalic anhydride (2.3 g, 15.2 mmol) in EtOH (50 mL) was heated at reflux for 3 h. The mixture was concentrated and the residue was purified by chromatography (DCM/MeOH=100:1) to give the product (8.1 g, 62% yield) as an off-white solid. LCMS (ES-API): Rₜ 2.12 min; m/z 310.1 [M+H]⁺.

### b) tert-Butyl (3-(1,3-dioxoisoindolin-2-yl)-2-phenylpropyl)carbamate (I43)

A solution of 4-(1,3-dioxoisoindolin-2-yl)-3-phenylbutanoic acid (I42) (8.1 g, 26.2 mmol), DPPA (7.9 g, 28.8 mmol), Boc₂O (11.4 g, 52.4 mmol) and TEA (13.2 g, 130.9 mmol) in t-BuOH/dioxane (30 mL/80 mL) was heated at 100° C. overnight. The mixture was concentrated and the residue was dissolved in EtOAc (200 mL), washed with water (3×100 mL), dried over Na₂SO₄, filtered and concentrated. The residue was purified by chromatography (Petroleum ether/EtOAc=10:1) to give the product (3.0 g, 30% yield) as a white solid. LCMS (ES-API): Rₜ 1.83 min; m/z 381.2 [M+H]⁺.

### c) tert-Butyl (3-amino-2-phenylpropyl) Carbamate (I44)

To a solution of tert-butyl (3-(1,3-dioxoisoindolin-2-yl)-2-phenylpropyl)carbamate (I43) (900 mg, 2.36 mmol) in EtOH (30 mL) was added N₂H₄.H₂O (120 mg, 2.36 mmol) and the mixture was heated at 80° C. for 2 h. The mixture was filtered and the solid was washed with more ethanol (2 mL). The combined filtrates were concentrated and the residue was purified by chromatography (DCM/MeOH=50:1) to give the product (300 mg, 51% yield) as yellow oil. LCMS (ES-API): Rₜ 0.83 min; m/z 251.2 [M+H]⁺.

### d) tert-Butyl (3-(1,1-dioxido-2H-benzo[e][1,2,4]thiadiazine-3-carboxamido)-2-phenylpropyl)carbamate (I45)

To a solution of tert-butyl (3-amino-2-phenylpropyl)carbamate (I44) (250 mg, 1.0 mmol) in DCM (20 mL) was added NaHCO₃ (840 mg, 10.0 mmol) and the mixture was stirred at r.t. for 10 min. 2H-Benzo[e][1,2,4]thiadiazine-3-carbonyl chloride 1,1-dioxide (I30) (1.23 g, 5.0 mmol) was added and stirring was continued at r.t. for 1 h. The mixture was diluted with DCM (30 mL) and washed with water (2×50 mL), 1 M aqueous HCl (50 mL), brine (50 mL), dried over Na₂SO₄, filtered and concentrated to give the product (300 mg, 66% yield) as a light yellow solid. LCMS (ES-API): Rₜ 2.27 min; m/z 459.2 [M+H]⁺.

### e) N-(3-Amino-2-phenylpropyl)-2H-benzo[e][1,2,4]thiadiazine-3-carboxamide 1,1-dioxide Hydrochloride (I46)

To a solution of tert-butyl (3-(1,1-dioxido-2H-benzo[e][1,2,4]thiadiazine-3-carboxamido)-2-phenylpropyl)carbamate (I45) (300 mg, 0.65 mmol) in EtOAc (1 mL) was added 2 M HCl in EtOAc (3 mL) and the mixture was stirred at r.t. for 2 h. The mixture was concentrated to give the product (220 mg, 85% yield) as an off-white solid. LCMS (ES-API): Rₜ 0.57 min; m/z 359.1 [M+H]⁺.

### (xiv) 4-(1,1-Dioxido-2H-benzo[e][1,2,4]thiadiazine-3-carboxamido)-3-phenylbutanoic Acid (I51)

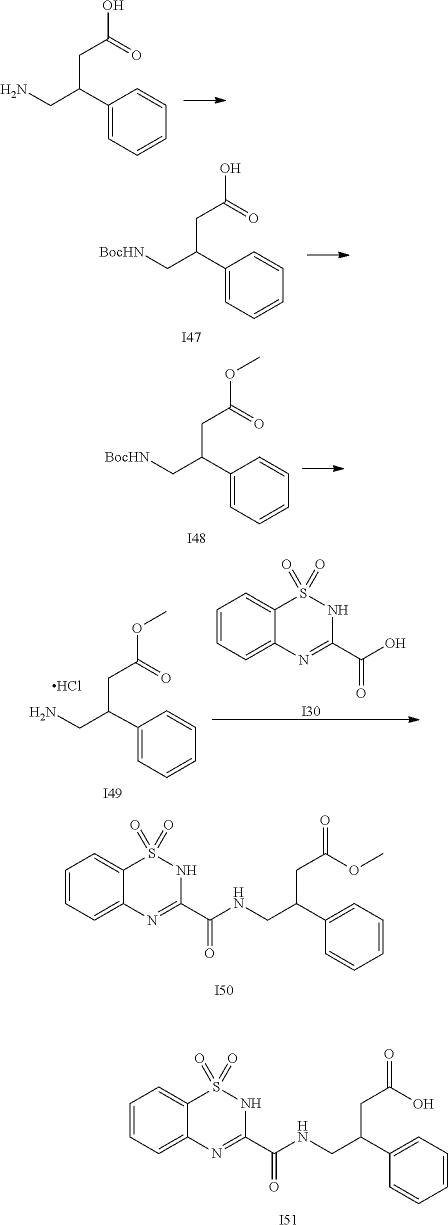

### a) 4-((tert-Butoxycarbonyl)amino)-3-phenylbutanoic Acid (I47)

To a solution of 4-amino-3-phenylbutanoic acid (3.0 g, 16.7 mmol) in 1 M aqueous NaOH (35 mL) and t-BuOH (25 mL) at 0° C. was added (Boc)₂O (3.65 g, 116.7 mmol) portion-wise and mixture was stirred at r.t. over the weekend. The mixture was washed with pentane (80 mL×2) and extracted with ether (80 mL×3). The combined ether extracts were dried over Na₂SO₄, filtered and concentrated to give the desired product (3.4 g, 73% yield) as a white solid. LCMS: Rₜ 2.43 min, m/z 302.1 [M+Na]⁺

### b) Methyl 4-((tert-butoxycarbonyl)amino)-3-phenylbutanoate (I48)

A mixture of 4-((tert-butoxycarbonyl)amino)-3-phenylbutanoic acid (I47) (2.793 g, 10 mmol) and K₂CO₃ (2.76 g, 20 mmol) in THF (50 mL) was stirred at r.t. for 15 min. Methyl iodide (3.01 g, 20 mmol) was then added and stirring was continued at r.t. overnight. The mixture was diluted with DCM (500 mL), washed with water (×2) and the organic phase was dried over Na₂SO₄, filtered and concentrated. The residue was purified by silica gel chromatography (Petroleum ether/EtOAc=100:1-30:1) to give the desired product (2.5 g, 85% yield) as a white solid. LCMS: Rₜ 12.16 min, m/z 316.2 [M+Na]⁺

### c) Methyl 4-amino-3-phenylbutanoate Hydrochloride (I49)

A mixture of methyl 4-((tert-butoxycarbonyl)amino)-3-phenylbutanoate (I48) (2.5 g, 8.52 mmol) and 2 M HCl/EtOAc (100 mL) was stirred at r.t. for 3 h. The solvent was removed and the residue was washed with EtOAc to give the desired product (1.5 g, 91% yield) as a white solid, which was used directly in the next step.

### d) Methyl 4-(1,1-dioxido-2H-benzo[e][1,2,4]thiadiazine-3-carboxamido)-3-phenylbutanoate (I50)

To a solution of methyl 4-amino-3-phenylbutanoate hydrochloride (I49) (1.5 g, 7.76 mmol) and 2H-benzo[e][1,2,4]thiadiazine-3-carboxylic acid 1,1-dioxide (I29) (2.63 g, 11.64 mmol) in DCM (100 mL) at r.t. was added triethylamine (3.14 g, 31.0 mmol) and HATU (4.43 g, 11.64 mmol) and the mixture was stirred at r.t. overnight. The solvent was removed and the residue was purified by silica gel chromatography (DCM/MeOH=100:0-100:1) to give the desired product (1.2 g, 58% yield) as a white solid. LCMS: Rₜ min, m/z 402 [M+H]⁺

### e) 4-(1,1-Dioxido-2H-benzo[e][1,2,4]thiadiazine-3-carboxamido)-3-phenylbutanoic Acid (I51)

A mixture of methyl 4-(1,1-dioxido-2H-benzo[e][1,2,4]thiadiazine-3-carboxamido)-3-phenylbutanoate (I50) (1.2 g, 3 mmol) in 2 M NaOH (100 mL) was stirred at r.t. for 3 h. The mixture was adjusted to pH 2-3 with conc. HCl and the resulting precipitate was collected by filtration, washed with twice with water and dried to give the desired product (600 mg, 52% yield) as a white solid. LCMS: Rₜ 12.16 min, m/z 388.1 [M+H]⁺

### (xv) (2-(2-Aminoethyl)phenyl)methanol (I52)

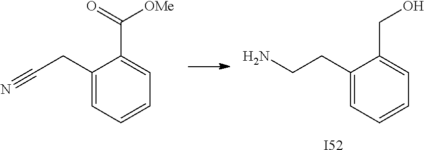 [SMILES: "I52" ; COC(=O)c1ccccc1CC#N ; NCCc1ccccc1CO]

To a solution of methyl 2-(cyanomethyl)benzoate (3 g, 17.1 mmol) in THF (50 mL) was added a 1 M solution of BH₃.THF in THF (51.3 mL, 51.3 mmol) and the mixture was heated at 70° C. under N₂ for 16 h. After cooling to r.t., the mixture was adjusted to pH 5 with 1 M HCl, diluted with water (20 mL) and washed with EtOAc (30 mL×3). The aqueous layer was adjusted to pH 9 with 1 M NaOH and then extracted with EtOAc (30 mL×3). The combined organic extracts were concentrated to give the product (1.5 g, 57% yield) as a yellow oil. LCMS (ES-API): Rₜ 2.34 min; m/z 152.1 [M+H]⁺.

### (xvi) 7-Iodo-2H-benzo[e][1,2,4]thiadiazine-3-carboxylic Acid 1,1-dioxide (I53)

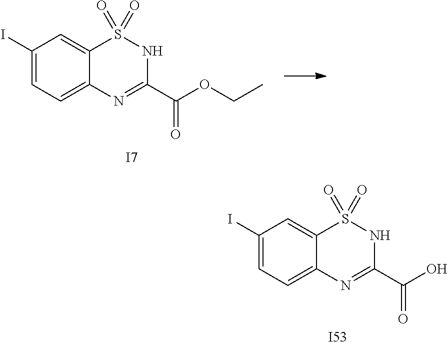

To a solution of ethyl 7-iodo-2H-benzo[e][1,2,4]thiadiazine-3-carboxylate 1,1-dioxide (I7) (200 mg, 0.53 mmol) in THF (10 mL), MeOH (1 mL) and H₂O (0.1 mL) was added LiOH.H₂O (67 mg, 1.59 mmol) and the mixture was stirred at r.t. overnight. Most of the organic solvent was removed under reduced pressure and the aqueous residue was adjusted to pH 5 with 1 M aq HCl and extracted with DCM (10 mL×3). The combined extracts were dried over Na₂SO₄ and concentrated to give the product (150 mg, 80% yield) as a yellow solid. LCMS (ES-API): Rₜ 1.0 min; m/z 353.1 [M+H]⁺.

### (xvii) N-(2-(hydroxymethyl)phenethyl)-7-iodo-2H-benzo[e][1,2,4]thiadiazine-3-carboxamide 1,1-dioxide (109)

See below

### (xviii) 2-(2-(7-Iodo-1,1-dioxido-2H-benzo[e][1,2,4]thiadiazine-3-carboxamido)ethyl)benzoic Acid (I55; 155)

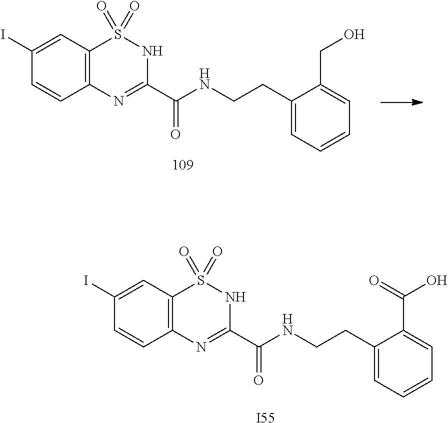 [SMILES: O=C(NCCc1ccccc1CO)C1=Nc2ccc(I)cc2S(=O)(=O)N1 ; "I55" ; O=C(NCCc1ccccc1C(=O)O)C1=Nc2ccc(I)cc2S(=O)(=O)N1]

To a solution of N-(2-(hydroxymethyl)phenethyl)-7-iodo-2H-benzo[e][1,2,4]thiadiazine-3-carboxamide 1,1-dioxide (109) (200 mg, 0.4 mmol) in acetone (10 mL) at r.t. was added

Jones reagent (10 mL) and the mixture was heated at 40° C. for 16 h then concentrated under reduced pressure. The residue was diluted with water (10 mL), the solid was collected by filtration, washed with diethyl ether (20 mL) and dried to give the product as a white solid (115 mg, 55% yield). ¹H NMR (400 MHz, d₆-DMSO) δ 12.8 (brs, 1H), 9.27 (m, 1H), 8.15-8.00 (m, 2H), 7.83 (m, 1H), 7.59 (d, J=6.4 Hz, 1H), 7.46 (m, 1H), 7.37-7.24 (m, 2H), 3.55 (m, 2H), 3.22 (m, 2H). LCMS (ES-API) Rₜ 2.72 min; m/z 497.6 [M−H]⁻.

### (xix) Ethyl 2-((4-fluoro-2-sulfamoylphenyl)amino)-2-oxoacetate (I56)

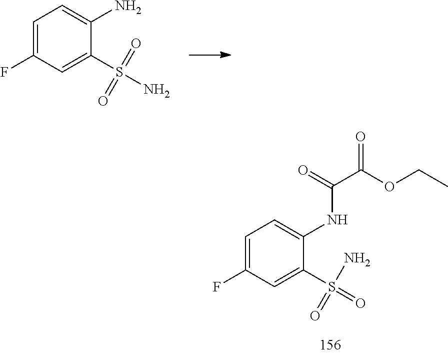 [SMILES: Nc1ccc(F)cc1S(N)(=O)=O ; CCOC(=O)C(=O)Nc1ccc(F)cc1S(N)(=O)=O]

To solution of 2-amino-5-fluorobenzenesulfonamide (0.200 g, 1.052 mmol) in THF (10 mL), at 0° C., was added NEt₃ (0.154 mL, 1.104 mmol) followed by the dropwise addition of ethyl chlorooxoacetate (0.123 mL, 1.104 mmol) over 10 min. The mixture was allowed to slowly warm to ambient temperature for 48 h. The precipitate was removed by filtration and the filtrate was concentrated in vacuo to give the product (0.320 g, 90% purity, 94% yield) as a white solid. LCMS-B: r.t. 3.059 min; m/z 289.0 [M−H]⁻. ¹H NMR (400 MHz, d-DMSO) δ 10.63 (s, 1H), 8.25 (dd, J=9.1, 4.9 Hz, 1H), 7.84 (s, 2H), 7.65 (dd, J=8.4, 3.0 Hz, 1H), 7.58 (ddd, J=9.1, 8.0, 3.1 Hz, 1H), 4.32 (q, J=7.1 Hz, 2H), 1.32 (t, J=7.1 Hz, 3H).

### (xx) Ethyl 7-fluoro-2H-benzo[e][1,2,4]thiadiazine-3-carboxylate (I57)

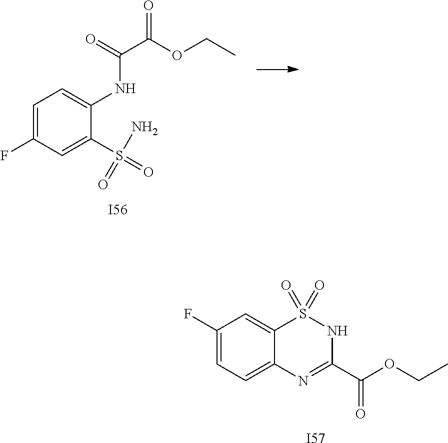 [SMILES: "I56" ; CCOC(=O)C(=O)Nc1ccc(F)cc1S(N)(=O)=O ; "I57" ; CCOC(=O)C1=Nc2ccc(F)cc2S(=O)(=O)N1]

To solution of ethyl 2-((4-fluoro-2-sulfamoylphenyl)amino)-2-oxoacetate (I56) (0.320 g, 90% purity, 0.992 mmol) in dry EtOH (10 mL) under an atmosphere of nitrogen, was added NaH (60% dispersion in mineral oil, 0.079 g, 1.984 mmol) in portion. The reaction was then stirred at room temperature for 20 h. The reaction was quenched with water (10 mL) and acidified to pH 3 with 1M HCl. The EtOH was removed in vacuo and the precipitate was collected by filtration. The solid was washed with water then air dried to give the desired product ethyl 7-fluoro-2H-benzo[e][1,2,4]thiadiazine-3-carboxylate 1,1-dioxide (0.069 g, 26% yield) as a white solid. LCMS-B: r.t. 3.409 min; m/z 271.0 [M−H]⁻. ¹H NMR (400 MHz, d-DMSO) δ 7.85 (dd, J=9.2, 4.6 Hz, 1H), 7.79 (dd, J=7.6, 2.8 Hz, 1H), 7.67 (td, J=8.8, 2.9 Hz, 1H), 4.40 (q, J=7.1 Hz, 2H), 1.35 (t, J=7.1 Hz, 3H).

### (xxi) (2-(2-(2-Aminoethyl)phenyl)-2H-1,2,3-triazol-4-yl)methanol (I60)

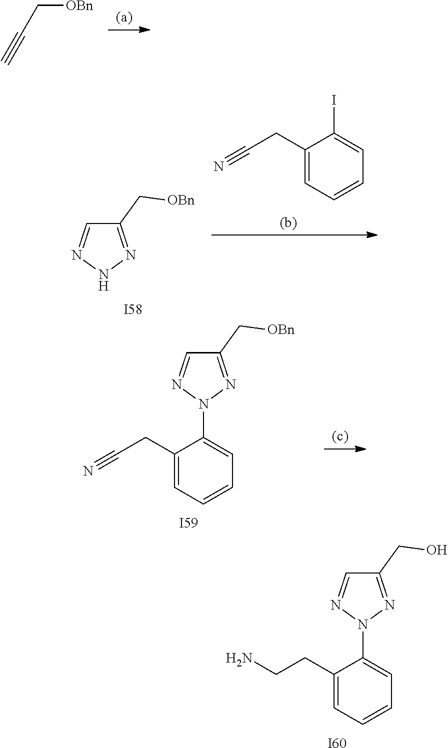

### a) 4-((Benzyloxy)methyl)-2H-1,2,3-triazole I58

To a solution of ((prop-2-yn-1-yloxy)methyl)benzene (1.46 g, 10.0 mmol) in DMF (20 mL) and EtOH (2.5 mL) was added CuI (380 mg, 2 mmol) and azidotrimethylsilane (2.3 g, 20 mmol) and the mixture was heated at 130° C. under N₂ for 18 h. The mixture was diluted with water and extracted with EtOAc (200 mL). The combined organic extracts were washed with water (100 mL×3), dried over anhydrous Na₂SO₄, filtered and concentrated under reduced pressure. The residue was purified by silica gel chromatography (Pet. ether/EtOAc=5/1) to give the title compound (900 mg, 50%) as a yellow oil. LCMS-D: Rₜ 1.42 min; m/z 190.1 [M+H]⁺.

### b) 2-(2-(4-((Benzyloxy)methyl)-2H-1,2,3-triazol-2-yl)phenyl)acetonitrile I59

A mixture of 4-((benzyloxy)methyl)-2H-1,2,3-triazole 158 (1.7 g, 9.0 mmol), 2-(2-iodophenyl)acetonitrile (3.0 g, 12.0 mmol), Fe(acac)₃ (1.1 g, 3.0 mmol), CuO (720 mg, 0.9 mmol) and Cs₂CO₃ (6.0 g, 18.0 mmol) in DMF (60 mL) was heated at 90° C. under N₂ for 30 h. The mixture was diluted with water and extracted with EtOAc. The combined organic extracts were washed with brine, dried over anhydrous Na₂SO₄, filtered and concentrated under reduced pressure to give the title compound (1.4 g, 51%) as a yellow oil. LCMS-D: Rₜ 2.87 min; m/z 305.1 [M+H]⁺.

### c) (2-(2-(2-Aminoethyl)phenyl)-2H-1,2,3-triazol-4-yl)methanol I60

To a solution of 2-(2-(4-((benzyloxy)methyl)-2H-1,2,3-triazol-2-yl)phenyl)acetonitrile 159 (700 mg, 2.3 mmol) in MeOH (30 mL) was added 10% Pd/C (200 mg) and the mixture was stirred at RT under a H₂ atmosphere overnight. The catalyst was removed by filtration through Celite and the filtrate was concentrated under reduced pressure. The residue was purified by silica gel chromatography (DCM/MeOH=10/0 to 10/1) to give the title compound (300 mg, 60%) as a yellow oil. LCMS-D: Rₜ 0.33 min; m/z 219.1 [M+H]⁺.

### xxii) 2-(5-(Difluoromethyl)-1,3,4-oxadiazol-2-yl)-2-phenylethan-1-amine Trifluoroacetate (I63)

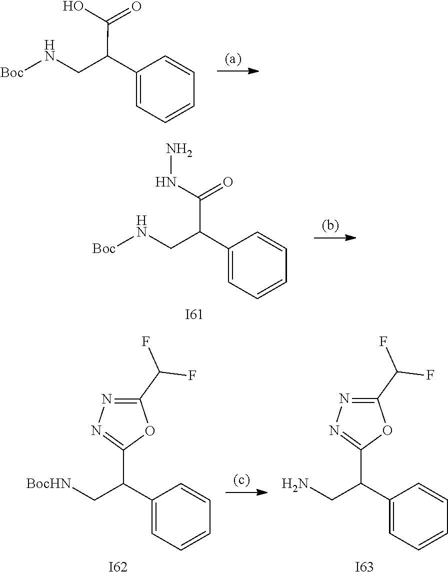

### a) tert-Butyl (3-hydrazinyl-3-oxo-2-phenylpropyl)carbamate I61

To a solution of 3-((tert-butoxycarbonyl)amino)-2-phenylpropanoic acid (2.65 g, 10.0 mmol) in dry THF (30 mL) was added CDI (1.93 g, 12.0 mmol) and the mixture was stirred at RT under N₂ for 90 min. Hydrazine monohydrate (1.5 g, 30.0 mmol) was then added and stirring was continued at RT for 18 h. The mixture was diluted with water and extracted with EtOAc (200 mL). The combined organic extracts were washed with water, dried over anhydrous Na₂SO₄, filtered and concentrated under reduced pressure to give the title compound (3.0 g, >100%) as a white solid, which was used in the next step without further purification. LCMS-D: Rₜ 2.29 min; m/z 302.0 [M+Na]⁺.

### b) tert-Butyl (2-(5-(difluoromethyl)-1,3,4-oxadiazol-2-yl)-2-phenylethyl)carbamate I62

A mixture of tert-butyl (3-hydrazinyl-3-oxo-2-phenylpropyl)carbamate I61 (240 mg, 0.86 mmol), trifluoroacetic anhydride (449 mg, 2.58 mmol) and imidazole (176 mg, 2.58 mmol) in DCM (10 mL) was heated at 50° C. under N₂ overnight. The reaction was quenched with a saturated aqueous NH₄Cl solution and the mixture was extracted with DCM (50 mL×3). The combined organic extracts were washed with a saturated aqueous NaHCO₃ solution, dried over anhydrous Na₂SO₄, filtered and concentrated under reduced pressure. The residue was purified by prep. TLC (DCM/MeOH=20/1) to give the title compound (170 mg, 58%) as a colorless oil. LCMS-D: Rₜ 2.69 min; m/z 362.0 [M+Na]⁺.

### c) 2-(5-(Difluoromethyl)-1,3,4-oxadiazol-2-yl)-2-phenylethan-1-amine Trifluoroacetate I63

To a solution of tert-butyl (2-(5-(difluoromethyl)-1,3,4-oxadiazol-2-yl)-2-phenylethyl)carbamate I62 (60 mg, 0.18 mmol) in DCM (3 mL) was added TFA (1.0 mL) and the mixture was stirred at RT for 2 h. The mixture was concentrated under reduced pressure to give the title compound (85 mg, >100%) as a yellow oil, which was used directly in the next step with further purification. LCMS-D: Rₜ 0.51 min; m/z 240.0 [M+H]⁺.

### xxiii) 2-Phenyl-2-(1,3,4-thiadiazol-2-yl)ethan-1-amine Hydrochloride (I66)

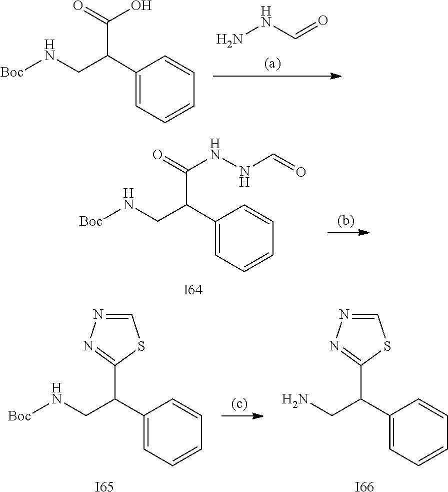

### a) tert-Butyl (3-(2-formylhydrazinyl)-3-oxo-2-phenylpropyl)carbamate I64

A mixture of 3-((tert-butoxycarbonyl)amino)-2-phenylpropanoic acid (2.0 g, 7.5 mmol), formic hydrazide (510 mg, 8.5 mmol), EDCI.HCl (2.1 g, 11.3 mmol), HOBt (2.0 g, 15.0 mmol) and Et₃N (2.3 g, 22.5 mmol) in DMF (30 mL) was stirred at RT overnight. The mixture was diluted with water and extracted with DCM. The combined organic extracts were washed with brine, dried over anhydrous Na₂SO₄, filtered and concentrated under reduced pressure. The residue was purified by silica gel chromatography (Pet. ether/EtOAc=30/1 to 10/1) to give the title compound (800 mg, 34%) as a yellow oil. LCMS-D: Rₜ 2.87 min; m/z 308.1 [M+H]⁺.

### b) tert-Butyl (2-phenyl-2-(1,3,4-thiadiazol-2-yl)ethyl)carbamate I65

To a solution of tert-butyl (3-(2-formylhydrazinyl)-3-oxo-2-phenylpropyl)carbamate I64 (600 mg, 1.95 mmol) in THF (30 mL) was added Lawesson's reagent (2.4 g, 5.85 mmol) and the mixture was heated at 40° C. overnight. The mixture was diluted with water and extracted with DCM. The combined organic extracts were washed with brine, dried over anhydrous Na₂SO₄, filtered and concentrated under reduced pressure. The residue was purified by prep. TLC (DCM/MeOH=30/1) to give the title compound (200 mg, 34%) as a yellow oil. LCMS-D: Rₜ 0.71 min; m/z 306.1 [M+H]⁺.

### c) 2-Phenyl-2-(1,3,4-thiadiazol-2-yl)ethan-1-amine Hydrochloride I66

To a solution of tert-butyl (2-phenyl-2-(1,3,4-thiadiazol-2-yl)ethyl)carbamate I65 (60 mg, 0.18 mmol) in DCM (10 mL) was added TFA (2.0 mL) and the mixture was stirred at RT overnight. 1 M aqueous HCl was added and the mixture was washed with EtOAc. The aqueous layer was concentrated under reduced pressure to give the title compound (260 mg, 98%) as a white solid. LCMS-CLCMS-C: Rₜ 10.62 min; m/z 206.1 [M+H]⁺.

### xxiv) 3-(Methylamino)-3-oxopropyl 3-amino-2-phenylpropanoate Hydrochloride (I69)

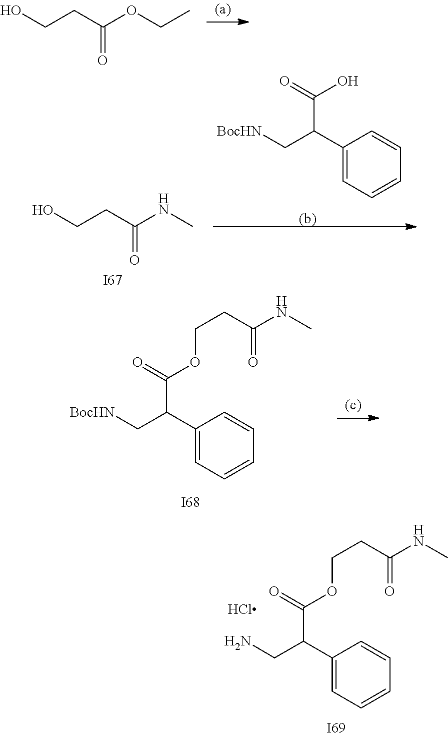

### a) 3-Hydroxy-N-methylpropanamide I67

A mixture of ethyl 3-hydroxypropanoate (2.0 g, 16.9 mmol) and MeNH₂ (30% (v/v) solution in methanol, 45 mL) was heated at 85° C. for 36 h. The mixture was concentrated under reduced pressure to give the title compound (1.5 g, 88%) as an oil. ¹H NMR (400 MHz, Chloroform-d) δ 7.28 (br s, 1H), 4.84 (br s, 1H), 3.82 (t, J=5.8 Hz, 2H), 2.75 (d, J=4.8 Hz, 3H), 2.42 (t, J=5.8 Hz, 2H).

### b) 3-(Methylamino)-3-oxopropyl 3-((tert-butoxycarbonyl)amino)-2-phenylpropanoate I68

A mixture of 3-((tert-butoxycarbonyl)amino)-2-phenylpropanoic acid (500 mg, 1.8 mmol), 3-hydroxy-N-methylpropanamide I67 (1.1 g, 9.5 mmol), EDCI.HCl (542 mg, 2.83 mmol) and DMAP (350 mg, 1.8 mmol) in DCM (100 mL) was stirred at RT overnight. The mixture was concentrated under reduced pressure and the residue was purified by silica gel chromatography to give the title compound (500 mg, 75%) as an oil. LCMS-D: Rₜ 2.13 min; m/z 251.3 [M-Boc+2H]⁺.

### c) 3-(Methylamino)-3-oxopropyl 3-amino-2-phenylpropanoate Hydrochloride I69

To a solution of 3-(methylamino)-3-oxopropyl 3-((tert-butoxycarbonyl)amino)-2-phenylpropanoate I68 (500 mg, 1.42 mmol) in DCM (30 mL) was added a 2 M solution of HCl in Et₂O (30 mL) and the mixture was stirred at RT overnight. The mixture was concentrated under reduced pressure and the residue was recrystallised from water and dried under reduced pressure to give the title compound (400 mg, 97%) as a white solid. LCMS-D: Rₜ 0.24 min; m/z 251.3 [M+H]⁺.

### xxv) 4-(Methylamino)-4-oxobutyl 3-amino-2-phenylpropanoate Trifluoroacetate (I72)

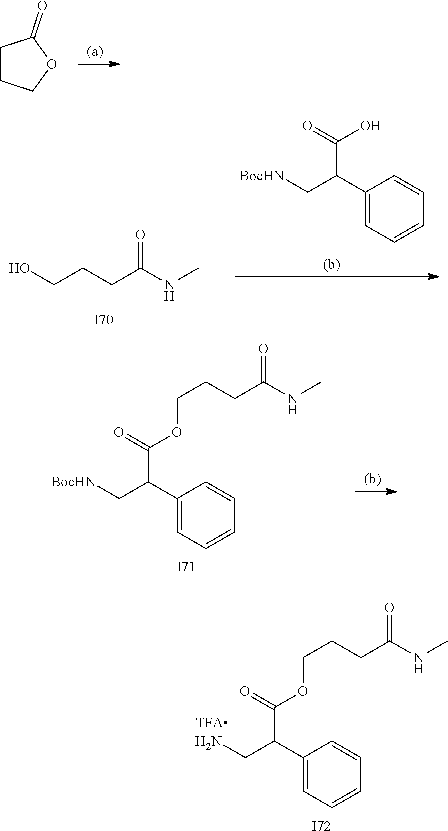

### a) 4-Hydroxy-N-methylbutanamide I70

Dihydrofuran-2(3H)-one (334 mg, 4.0 mmol) was added to a 2 M solution of methylamine in THF (20.0 mL, 40.0 mmol) in a pressure tube at −78° C. The flask was sealed and the mixture was stirred at RT overnight. The mixture was then concentrated under reduced pressure to give the title compound (350 mg, 75%) as a red solid. LCMS-CLCMS-C: Rₜ 0.33 min; m/z 118.1 [M+H]⁺.

### b) 4-(Methylamino)-4-oxobutyl 3-((tert-butoxycarbonyl)amino)-2-phenylpropanoate I71

A mixture of 3-((tert-butoxycarbonyl)amino)-2-phenylpropanoic acid (500 mg, 1.88 mmol), 4-hydroxy-N-methylbutanamide I70 (331 mg, 2.83 mmol), EDCI.HCl (434 mg, 2.26 mmol) and DMAP (23 mg, 0.19 mmol) in DCM (20 mL) was stirred at RT overnight. The mixture was diluted with water (100 mL), extracted with DCM (60 mL×3) and the combined organic extracts were washed with brine, dried over anhydrous Na₂SO₄, filtered and concentrated. The residue was purified by prep. TLC (DCM/MeOH=300/1 to 100/1) to give the title compound (400 mg, 80%) as a yellow oil. LCMS-D: Rₜ 1.85 min; m/z 387.1 [M+Na]⁺, 265.1 [M-Boc+2H]⁺.

### c) 4-(Methylamino)-4-oxobutyl 3-amino-2-phenylpropanoate Trifluoroacetate I72

To a solution of 4-(methylamino)-4-oxobutyl 3-((tert-butoxycarbonyl)amino)-2-phenylpropanoate I71 (220 mg, 0.55 mmol) in DCM (2 mL) was added TFA (1.0 mL) and the mixture was stirred at RT for 3 h. The mixture was concentrated under reduced pressure to give the title compound (330 mg, >100%) as a yellow oil, which was used in the next step without further purification. LCMS-D: Rₜ 0.31 min; m/z 265.1 [M+H]⁺ for the free base.

### xxvi) 2-(2-Methoxyphenyl)-2-(oxazol-2-yl)ethan-1-amine (I76)

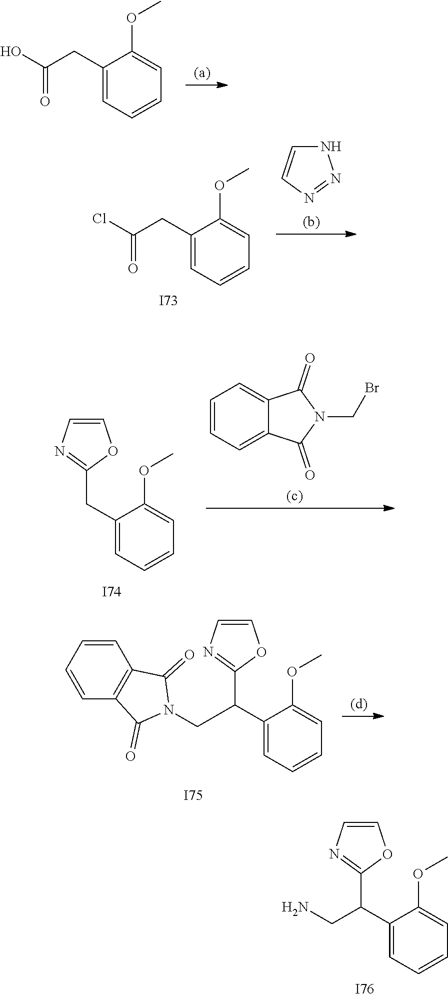

### a) 2-(2-Methoxyphenyl)acetyl Chloride I73

To a solution of 2-(2-methoxyphenyl)acetic acid (10 g, 60.2 mmol) in DCM (100 mL) was added oxalyl chloride (15 mL, 180.5 mmol) dropwise followed by DMF (3 drops) and the mixture was stirred at RT under N₂ for 2 h. The mixture was concentrated under reduced pressure to give the title compound (11 g, 100%) as a red oil. LCMS-D: Rₜ 2.28 min; m/z 181.0 [M−Cl+MeOH]⁺.

### b) 2-(2-Methoxybenzyl)oxazole I74

To a mixture of 1,2,3-triazole (5.4 g, 78.3 mmol) and K₂CO₃ (13.5 g, 97.8 mmol) in sulfolane (100 mL) at 0° C. was added 2-(2-methoxyphenyl)acetyl chloride I73 (12 g, 65.2 mmol) and the mixture was heated at 165° C. for 1 h. After cooling to RT, the mixture was diluted with water (500 mL) and extracted with Et₂O (500 mL×3). The combined organic extracts were washed with water (500 mL×3), brine, dried over anhydrous Na₂SO₄, filtered and concentrated under reduced pressure. The residue was purified by silica gel chromatography (Pet. ether/EtOAc=20/1 to 6/1) to give the title compound (8.0 g, 65%) as a yellow oil. LCMS-D: Rₜ 2.36 min; m/z 190.0 [M+H]⁺, 212.0 [M+Na]⁺.

### c) 2-(2-(2-Methoxyphenyl)-2-(oxazol-2-yl)ethyl)isoindoline-1,3-dione I75

To a solution of 2-(2-methoxybenzyl)oxazole I74 (1.0 g, 5.3 mmol) in dry THF (20 mL) at −78° C. under N₂ was added LiHMDS (1 M solution in THF, 6.4 mL, 6.4 mmol) dropwise. The mixture was stirred at −78° C. for 1 h, then added to a solution 2-(bromomethyl)isoindoline-1,3-dione (1.5 g, 6.34 mmol) in dry THF (20 mL) at −78° C. under N₂. The mixture was allowed to warm to RT and stirred overnight. The reaction was quenched with a saturated aqueous NH₄Cl solution and the mixture was extracted with DCM (200 mL×3). The combined organic extracts were washed with brine, dried over anhydrous Na₂SO₄, filtered and concentrated under reduced pressure. The residue was purified by silica gel chromatography (Pet. ether/EtOAc=20/1 to 6/1) to give the title compound (200 mg, 11%) as a green solid. LCMS-D: Rₜ 2.50 min; m/z 349.0 [M+H]⁺.

### d) 2-(2-Methoxyphenyl)-2-(oxazol-2-yl)ethan-1-amine I76

A suspension of 2-(2-(2-methoxyphenyl)-2-(oxazol-2-yl)ethyl)isoindoline-1,3-dione I75 (200 mg, 0.57 mmol) and hydrazine hydrate (86 mg, 1.72 mmol) in EtOH (10 mL) was heated at 80° C. under N₂ for 3 h. The mixture was filtered and the filter cake was washed with EtOH (2 mL). The filtrate was concentrated under reduced pressure to give the title compound (100 mg, 80%) as a yellow oil. LCMS-D: Rₜ 0.41 min; m/z 219.1 [M+H]⁺.

### xxvii) 2-(2-(Difluoromethoxy)phenyl)-2-(oxazol-2-yl)ethan-1-amine (I80)

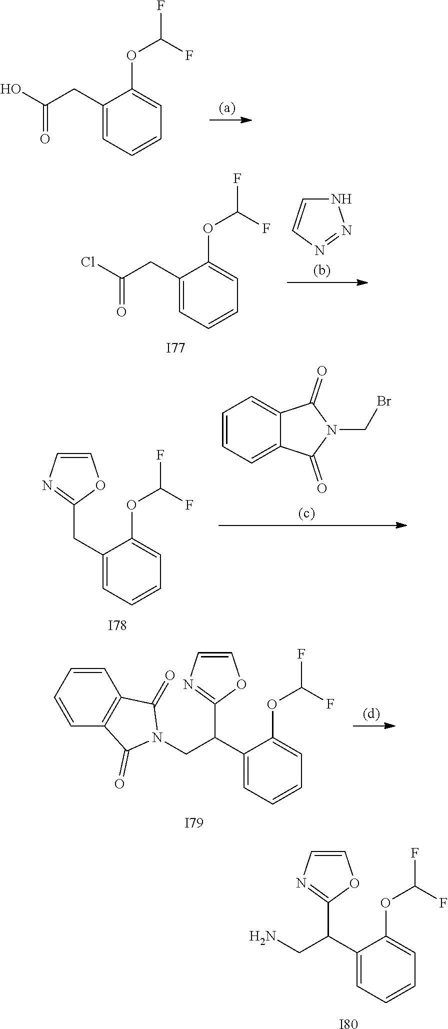

### a) 2-(2-Isopropoxyphenyl)acetyl Chloride I77

To a solution of 2-(2-(difluoromethoxy)phenyl)acetic acid (2.0 g, 9.89 mmol) in DCM (20 mL) was added oxalyl chloride (3 mL, 29.67 mmol) dropwise followed by DMF (3 drops) and the mixture was stirred at RT for 3 h. The mixture was concentrated under reduced pressure to give the title compound (2.2 g, 100%) as a red oil. LCMS-D: Rₜ 2.02 min; m/z 239.0 [M−Cl+MeO+Na]⁺

### b) 2-(2-(Difluoromethoxy)benzyl)oxazole I78

To a mixture of 1,2,3-triazole (1.0 g, 4.53 mmol) and K₂CO₃ (0.94 g, 6.80 mmol) in sulfolane (30 mL) at 0° C. was added 2-(2-isopropoxyphenyl)acetyl chloride I77 (1.0 g, 4.53 mmol) and the mixture was heated at 165° C. under N₂ for 1 h. After cooling to RT, the mixture was diluted with water (100 mL) and extracted with Et₂O (100 mL×3). The combined organic extracts were washed with water (100 mL), brine (100 mL), dried over anhydrous Na₂SO₄, filtered and concentrated under reduced pressure. The residue was purified by silica gel chromatography (Pet. ether/EtOAc=20/1 to 6/1) to give the title compound (800 mg, 78%) as a yellow oil. LCMS-D: Rₜ 1.74 min; m/z 226.0 [M+H]⁺.

### c) 2-(2-(2-(Difluoromethoxy)phenyl)-2-(oxazol-2-yl)ethyl)isoindoline-1,3-dione I79

To a solution of 2-(2-(difluoromethoxy)benzyl)oxazole I78 (1.1 g, 4.88 mmol) in dry THF (30 mL) at −78° C. under N₂ was added LiHMDS (1 M solution in THF, 6.0 mL, 6.0 mmol) dropwise. The mixture was stirred at −78° C. for 1 h, then added to a solution of 2-(bromomethyl)isoindoline-1,3-dione (1.41 g, 5.86 mmol) in dry THF (20 mL) at −78° C. under N₂. The mixture was allowed to warm to RT and stirred overnight. The reaction was quenched with a saturated aqueous NH₄Cl solution (50 mL) and the mixture was extracted with DCM (50 mL×3). The combined organic extracts were washed with brine (50 mL), dried over anhydrous Na₂SO₄, filtered and concentrated under reduced pressure. The residue was purified by silica gel chromatography (Pet. ether/EtOAc=20/1 to 6/1) to give the title compound (360 mg, 19%) as a yellow solid. LCMS-D: Rₜ 2.21 min; m/z 385.0 [M+H]⁺.

### d) 2-(2-(Difluoromethoxy)phenyl)-2-(oxazol-2-yl)ethan-1-amine I80

A suspension of 2-(2-(2-(difluoromethoxy)phenyl)-2-(oxazol-2-yl)ethyl)isoindoline-1,3-dione I79 (360 mg, 0.94 mmol) and hydrazine hydrate (0.15 mL, 2.81 mmol) in EtOH (20 mL) was heated at 80° C. under N₂ for 3 h. The mixture was filtered and the filter cake was washed with EtOH (2 mL). The filtrate was concentrated under reduced pressure to give the title compound (150 mg, 63%) as a yellow oil. LCMS-D: Rₜ 0.34 min; m/z 255.0 [M+H]⁺

### xxviii) 2-(5-(Methoxymethyl)-1,3,4-oxadiazol-2-yl)-2-phenylethan-1-amine trifluoroacetate (I84)

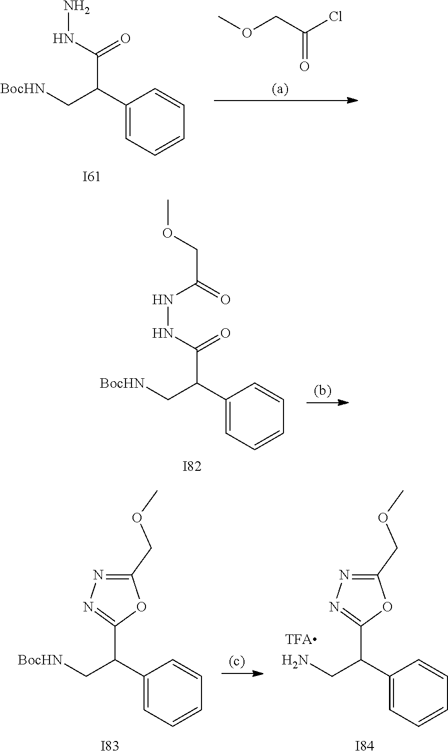

### a) tert-Butyl (3-(2-(2-methoxyacetyl)hydrazinyl)-3-oxo-2-phenylpropyl)carbamate I82

To a solution of tert-butyl (3-hydrazinyl-3-oxo-2-phenylpropyl)carbamate I61 (515 mg, 1.84 mmol) in THF (50 mL) was added pyridine (292 mg, 3.69 mmol) and 2-methoxyacetyl chloride (240 mg, 2.21 mmol) and the mixture was stirred at RT overnight. The mixture was concentrated under reduced pressure and the residue was diluted with water (100 mL) and extracted with DCM (100 mL×3). The combined organic extracts were washed with brine (100 mL), dried over anhydrous Na₂SO₄, filtered and concentrated under reduced pressure. The residue was purified by prep. TLC (DCM/MeOH=20/1) to give the title compound (230 mg, 36%) as a yellow oil. LCMS-CLCMS-C: Rₜ 1.60 min; m/z 352.0 [M+H]⁺.

### b) tert-Butyl (2-(5-(methoxymethyl)-1,3,4-oxadiazol-2-yl)-2-phenylethyl)carbamate I83

To a solution of tert-butyl (3-(2-(2-methoxyacetyl)hydrazinyl)-3-oxo-2-phenylpropyl)carbamate I82 (30 mg, 0.085 mmol) in THF (2 mL) was added Burgess reagent (41 mg, 0.17 mmol) and the mixture was heated at 120° C. under microwave irradiation for 30 min. The procedure was repeated once on the same scale and once using tert-butyl (3-(2-(2-methoxyacetyl)hydrazinyl)-3-oxo-2-phenylpropyl)carbamate I82 (150 mg, 0.60 mmol) and Burgess reagent (711 mg, 2.98 mmol) in THF (3 mL). The three reaction mixtures were combined, diluted with water (50 mL) and extracted with DCM (50 mL×3). The combined organic extracts were washed with brine (40 mL), dried over anhydrous Na₂SO₄, filtered and concentrated under reduced pressure. The residue was purified by prep. TLC (DCM/MeOH=20/1) to give the title compound (70 mg, 27%) as a yellow oil. LCMS-D: Rₜ 1.96 min; m/z 356.0 [M+Na]⁺.

### c) 2-(5-(Methoxymethyl)-1,3,4-oxadiazol-2-yl)-2-phenylethan-1-amine trifluoroacetate I84

A solution of tert-butyl (2-(5-(methoxymethyl)-1,3,4-oxadiazol-2-yl)-2-phenylethyl)carbamate I83 (70 mg, 0.21 mmol) and TFA (2 mL) in DCM (1 mL) was stirred at RT for 2 h. The mixture was concentrated under reduced pressure to give the title compound (60 mg, 82%) as a yellow oil, which was used in the next step without further purification. LCMS-C: Rₜ 0.87 min; m/z 233.9 [M+H]⁺ for the free base.

### xxix) 2-(3-Iodophenyl)-2-(oxazol-2-yl)ethan-1-amine (I88)

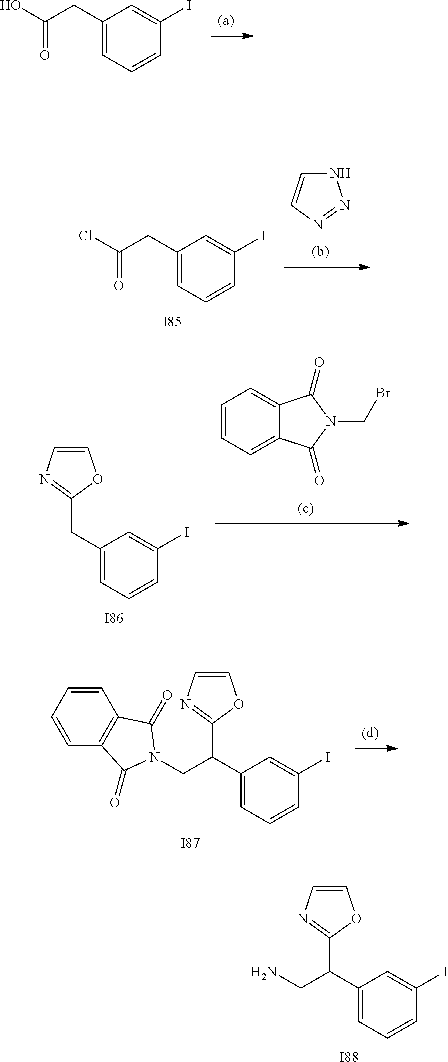

### a) 2-(3-Iodophenyl)acetyl Chloride I85

To a solution of 2-(3-iodophenyl)acetic acid (10.0 g, 38 mmol) in DCM (50 mL) was added oxalyl chloride (10.0 mL, 115 mmol) and DMF (1 mL) and the mixture was stirred at RT for 5 h. The mixture was concentrated under reduced pressure to give the title compound (10.0 g, 94%) as a yellow oil, which was used directly in the next step.

### b) 2-(3-Iodobenzyl)oxazole I86

To a mixture of 1,2,3-triazole (3.0 g, 43.2 mmol) and K₂CO₃ (7.3 g, 53.0 mmol) in sulfolane (80 mL) was added a solution of 2-(3-iodophenyl)acetyl chloride I85 (10.0 g, 36.0 mmol) in sulfolane (20 mL) and the mixture was heated at 165° C. under N₂ for 1 h. After cooling to RT, the mixture was diluted with water and extracted with Et₂O. The combined organic extracts were concentrated under reduced pressure and the residue was purified by silica gel chromatography (Pet. ether/EtOAc=50/1 to 20/1 to 10/1) to give the title compound (6.0 g, 58%) as a yellow oil. LCMS-C: Rₜ 2.13 min; m/z 285.9 [M+H]⁺.

### c) 2-(2-(3-Iodophenyl)-2-(oxazol-2-yl)ethyl)isoindoline-1,3-dione I87

To a solution of 2-(3-iodobenzyl)oxazole I86 (6.0 g, 21 mmol) in dry THF (100 mL) at −78° C. under N₂ was added LiHMDS (1 M solution in THF, 25.0 mL, 25.0 mmol) dropwise and the mixture was stirred at −78° C. for 45 min. A solution of 2-(bromomethyl)isoindoline-1,3-dione (6.0 g, 25.0 mmol) in dry THF (60 mL) was then added dropwise at −78° C. and the mixture was allowed to warm to RT and stirred overnight. The mixture was diluted with water, extracted with EtOAc and the combined organic extracts were concentrated under reduced pressure. The residue was purified by silica gel chromatography (Pet. ether/EtOAc=30/1 to 10/1) to give the title compound (1.8 g, 19%) as a yellow oil. LCMS-C: Rₜ 2.33 min; m/z 445.1 [M+H]⁺.

### d) 2-(3-Iodophenyl)-2-(oxazol-2-yl)ethan-1-amine I88

A suspension of 2-(2-(3-iodophenyl)-2-(oxazol-2-yl)ethyl)isoindoline-1,3-dione I87 (1.8 g, 4.0 mmol) and hydrazine monohydrate (600 mg, 12.0 mmol) in EtOH (30 mL) was heated at 80° C. under N₂ overnight. After cooling to RT, the mixture was diluted with water and extracted with DCM. The combined organic extracts were concentrated under reduced pressure to give the title compound (760 mg, 63%) as a yellow oil. LCMS-C: Rₜ 0.36 min; m/z 315.1 [M+H]⁺. ¹H NMR (400 MHz, DMSO-d₆) δ 8.02 (s, 1H), 7.64-7.60 (m, 2H), 7.28-7.24 (m, 1H), 7.19 (s, 1H), 7.13 (t, J=8.0 Hz, 1H), 4.22-4.16 (m, 1H), 3.25-3.18 (m, 1H), 3.04-2.98 (m, 1H).

### xxx) 5-(2-Amino-1-phenylethyl)-1,3,4-oxadiazol-2-amine hydrochloride (I90)

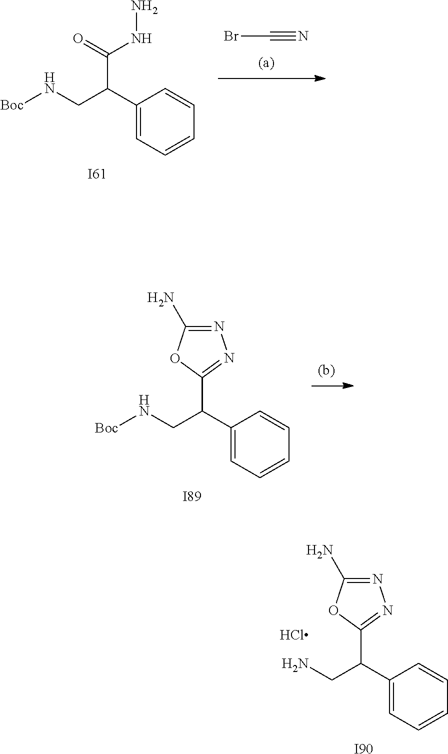

### a) tert-Butyl(2-(5-amino-1,3,4-oxadiazol-2-yl)-2-phenylethyl)carbamate I89

To a solution of tert-butyl(3-hydrazinyl-3-oxo-2-phenylpropyl)carbamate I61 (130 mg, 0.5 mmol) in 1,4-dioxane (5 mL) was added a solution of NaHCO₃ (42 mg, 0.5 mmol) in water (1.5 mL) and a white suspension was formed. Bromoacetonitrile (53 mg, 0.5 mmol) was then added portion wise and the mixture was stirred at RT overnight. The reaction was scaled up accordingly using tert-butyl(3-hydrazinyl-3-oxo-2-phenylpropyl)carbamate (1 mmol) and the reaction mixtures were combined, concentrated under reduced pressure to remove most of the 1,4-dioxane and the aqueous residue was extracted with EtOAc (100 mL). The organic extract was washed with a saturated aqueous NaHCO₃ solution, dried over Na₂SO₄, filtered and concentrated under reduced pressure to give the title compound (400 mg, 88%) as a white solid. LCMS-DLCMS-D: Rₜ 2.38 min, m/z 305.1 [M+H]⁺.

### b) 5-(2-Amino-1-phenylethyl)-1,3,4-oxadiazol-2-amine hydrochloride I90

A mixture of tert-butyl(2-(5-amino-1,3,4-oxadiazol-2-yl)-2-phenylethyl)carbamate I89 (183 mg, 0.6 mmol) and a 2 M solution of HCl in 1,4-dioxane (10 mL) was stirred at RT under N₂ for 2 h. The mixture was then concentrated under reduced pressure to give the title compound (120 mg, 83%) as a white solid. LCMS-D: Rₜ 0.28 min, m/z 205.1 [M+H]⁺.

### xxxi) 5-(2-Amino-1-phenylethyl)-1,3,4-oxadiazol-2(3H)-one Hydrochloride I92

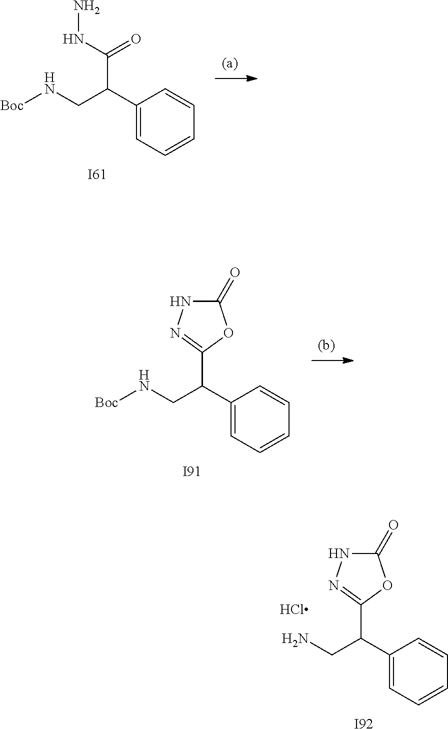 [SMILES: "I61" ; CC(C)(C)OC(=O)NCC(C(=O)NN)c1ccccc1 ; "I91" ; CC(C)(C)OC(=O)NCC(c1ccccc1)c1n[nH]c(=O)o1 ; "I92" ; NCC(c1ccccc1)c1n[nH]c(=O)o1]

### a) tert-Butyl (2-(5-oxo-4,5-dihydro-1,3,4-oxadiazol-2-yl)-2-phenylethyl)carbamate I91

To a solution of tert-butyl(3-hydrazinyl-3-oxo-2-phenylpropyl)carbamate I61 (320 mg, 1.15 mmol) and DIPEA (297 mg, 2.3 mmol) in DCM (12 mL) at 0° C. under N₂ was added a solution of triphosgene (137 mg, 0.46 mmol) in DCM (8 mL) and the mixture was stirred for 15 min, then allowed to warm to RT and stirred overnight. The mixture was diluted with DCM (50 mL), washed with a saturated aqueous NaHCO₃ solution, dried over Na₂SO₄, filtered and concentrated under reduced pressure. The residue was purified by prep. TLC (DCM/MeOH=20/1) to give the title compound (170 mg, 49%) as a white solid. LCMS-D: Rₜ 2.43 min, m/z 328.0 [M+Na]⁺.

### b) 5-(2-Amino-1-phenylethyl)-1,3,4-oxadiazol-2(3H)-one Hydrochloride I92

A mixture of tert-butyl (2-(5-oxo-4,5-dihydro-1,3,4-oxadiazol-2-yl)-2-phenylethyl)carbamate I91 (110 mg, 0.36 mmol) and a 2 M solution of HCl in 1,4-dioxane (10 mL) was stirred at RT overnight. The mixture was then concentrated under reduced pressure to give the title compound (110 mg, >100%) as a white solid, which was used directly in the next step. LCMS-D: Rₜ 0.27 min, m/z 206.1 [M+H]⁺.

### xxxii) 2-(3-Methoxyphenyl)-2-(oxazol-2-yl)ethan-1-amine (I96)

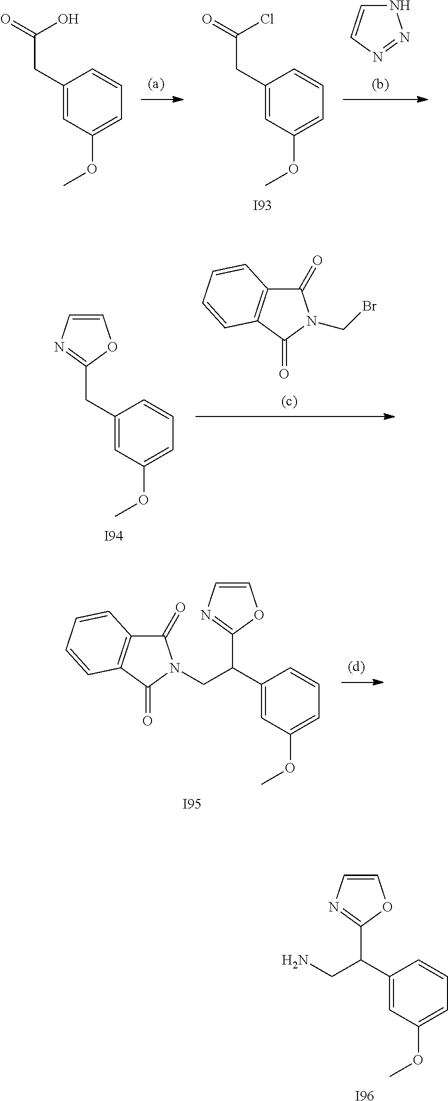

### a) 2-(3-Methoxyphenyl)acetyl Chloride I93

To a solution of 2-(3-methoxyphenyl)acetic acid (10.0 g, 60.0 mmol) and DMF (3 drops) in DCM (100 mL) at 0° C. under N₂ was added oxalyl chloride (23.0 g, 180 mmol) and the mixture was stirred for 3 h. The solvent was removed under reduced pressure to give the title compound (11.0 g, 100%) as a yellow oil. LCMS-D: Rₜ 2.17 min, m/z 181.0 [M−Cl+MeO+H]⁺.

### b) 2-(3-Methoxybenzyl)oxazole I94

To a mixture of 1,2,3-triazole (5.00 g, 72.0 mmol) and K₂CO₃ (13.0 g, 90.0 mmol) in sulfolane (150 mL) at 0° C. was added 2-(3-methoxyphenyl)acetyl chloride I93 (11.0 g, 60.0 mmol) dropwise and the mixture was heated at 165° C. for 1 h. After cooling to RT, MTBE (400 mL) was added and the mixture was washed with water (500 mL×3), dried over Na₂SO₄, filtered and concentrated under reduced pressure. The residue was purified by silica gel chromatography (Pet. ether/EtOAc=20/1) to give the title compound (5.2 g, 50%) as a yellow oil. LCMS-D: Rₜ 2.24 min, m/z 190.0 [M+H]⁺.

### c) 2-(2-(3-Methoxyphenyl)-2-(oxazol-2-yl)ethyl)isoindoline-1,3-dione I95

To a solution of 2-(3-methoxybenzyl)oxazole I94 (5.2 g, 27.5 mmol) in dry THF (80 mL) at −78° C. under N₂ was added LiHMDS (1 M solution in THF, 33.0 mL, 33.0 mmol) dropwise. The mixture was stirred at −78° C. for 45 min, then added to a solution of 2-(bromomethyl)isoindoline-1,3-dione (7.9 g, 33 mmol) in dry THF (120 mL) at −78° C. under N₂ and the mixture was stirred at −78° C. overnight. The solvent was removed under reduced pressure and the residue was diluted with DCM (200 mL), washed with a saturated aqueous NaHCO₃ solution (100 mL), dried over Na₂SO₄, filtered and concentrated under reduced pressure. The residue was purified by silica gel chromatography (Pet. ether/EtOAc=4/1) to give the title compound (2.69 g, 28%) as a yellow solid. LCMS-D: Rₜ 2.58 min, m/z 349.1 [M+H]⁺.

### d) 2-(3-Methoxyphenyl)-2-(oxazol-2-yl)ethan-1-amine I96

A suspension of 2-(2-(3-methoxyphenyl)-2-(oxazol-2-yl)ethyl)isoindoline-1,3-dione 195 (2.69 g, 7.70 mmol) and hydrazine monohydrate (1.20 g, 23.0 mmol) in EtOH (50 mL) was stirred at 80° C. under N₂ for 3 h. The mixture was then filtered and the filtrate was concentrated under reduced pressure to give the title compound (1.4 g, 80%) as a yellow oil. LCMS-D: Rₜ 0.43 min, m/z 219.0 [M+H]⁺. ¹H NMR (400 MHz, DMSO-d₆) δ 8.04-7.96 (m, 1H), 7.23 (t, J=8.0 Hz, 1H), 7.18 (s, 1H), 6.87-6.74 (m, 3H), 4.16 (dd, J=8.3, 6.2 Hz, 1H), 3.72 (s, 3H), 3.28-3.19 (m, 1H), 3.06-2.98 (m, 1H).

### xxxiii) 2-Fluoro-2-(oxazol-2-yl)-2-phenylethanamine (I99)

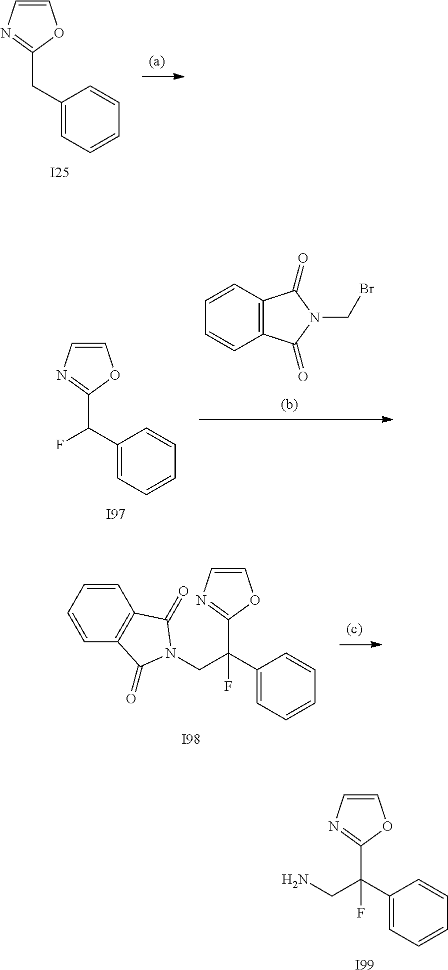

### a) 2-(Fluoro(phenyl)methyl)oxazole I97

To a solution of 2-benzyloxazole 125 (15.1 g, 95.0 mmol) in dry THF (150 mL) at −78° C. under N₂ was added t-BuLi (1.3 M solution in heptane, 81.0 mL, 105 mmol) dropwise. The mixture stirred at −78° C. for 45 min, then added to a solution of N-fluorobenzenesulfonimide (39.0 g, 124 mmol) in dry THF (100 mL) at −78° C. under N₂ and the mixture was stirred at −78° C. overnight. The reaction was quenched with a saturated aqueous NH₄Cl solution (100 mL) and the mixture was extracted with EtOAc (300 mL). The organic extract was dried over Na₂SO₄, filtered and concentrated under reduced pressure. The residue was purified by silica gel chromatography (Pet. ether/EtOAc=28/1) to give the title compound (10.2 g, 63%) as a red oil. LCMS-D: Rₜ 1.25 min, m/z 178.0 [M+H]⁺. ¹H NMR (400 MHz, DMSO-d₆) δ 8.21 (s, 1H), 7.55-7.41 (m, 5H), 7.32 (s, 1H), 6.84 (d, J=24.0 Hz, 1H).

### b) 2-(2-Fluoro-2-(oxazol-2-yl)-2-phenylethyl)isoindoline-1,3-dione I98

To a solution of 2-(fluoro(phenyl)methyl)oxazole I97 (3.54 g, 20 mmol) in dry THF (30 mL) at −78° C. under N₂ was added LiHMDS (1 M solution in THF, 24.0 mL, 24.0 mmol) dropwise. The mixture was stirred at −78° C. for 45 min, then added to a solution of 2-(bromomethyl)isoindoline-1,3-dione (5.76 g, 24.0 mmol) in dry THF (60 mL) at −78° C. under N₂ and the mixture was stirred at −78° C. overnight. The mixture was diluted with water, extracted with EtOAc and the organic layer was dried over Na₂SO₄, filtered and concentrated under reduced pressure. The residue was purified by silica gel chromatography (Pet. ether/EtOAc=5/1) to give the title compound (520 mg, 8%) as a white solid. LCMS-D: Rₜ 2.12 min, m/z 337.0 [M+H]⁺.

### c) 2-Fluoro-2-(oxazol-2-yl)-2-phenylethanamine I99

A suspension of 2-(2-fluoro-2-(oxazol-2-yl)-2-phenylethyl)isoindoline-1,3-dione I98 (520 mg, 1.5 mmol) and hydrazine monohydrate (225 mg, 4.5 mmol) in EtOH (10 mL) was heated at 80° C. under N₂ for 3 h. The mixture was concentrated under reduced pressure and the residue was dissolved in EtOAc (100 mL), washed with water (50 mL×3), dried over Na₂SO₄, filtered and concentrated under reduced pressure to give the title compound (250 mg, 80%) as a yellow oil. LCMS-D: Rₜ 0.28 min, m/z 207.0 [M+H]⁺. ¹H NMR (400 MHz, DMSO-d₆) δ 8.21-8.16 (m, 1H), 7.48-7.26 (m, 6H), 3.58-3.44 (m, 1H), 3.39-3.25 (m, 1H).

### xxxiv) 2-Phenyl-2-(5-(2,2,2-trifluoroethyl)-1,3,4-oxadiazol-2-yl)ethanamine (I102)

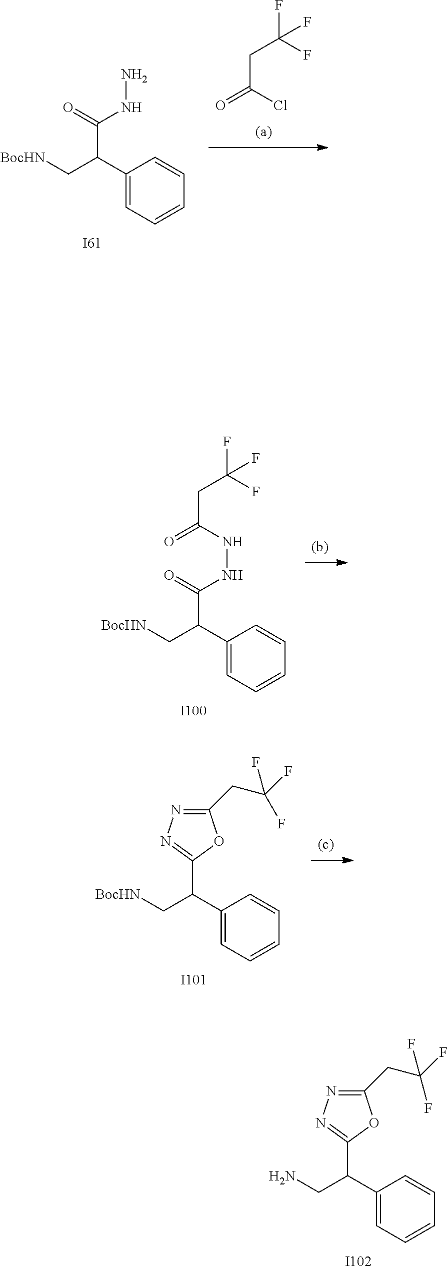

### a) tert-Butyl (3-oxo-2-phenyl-3-(2-(3,3,3-trifluoropropanoyl)hydrazinyl)propyl)carbamate I100

To a solution of tert-butyl (3-hydrazinyl-3-oxo-2-phenylpropyl)carbamate I61 (558 mg, 2.0 mmol) and pyridine (320 mg, 4.0 mmol) in dry THF (20 mL) at RT was added a solution of 3,3,3-trifluoropropanoyl chloride (580 mg, 4.0 mmol) in dry THF (5 mL) dropwise and the mixture was stirred for 2 h. The mixture was concentrated under reduced pressure and the residue was diluted with EtOAc (50 mL), washed with 1 M aqueous HCl, dried over Na₂SO₄, filtered and concentrated under reduced pressure to give the title compound (610 mg, 80%) as a white solid. LCMS-D: Rₜ 1.62 min, m/z 412.1 [M+Na]⁺.

### b) tert-Butyl (2-phenyl-2-(5-(2,2,2-trifluoroethyl)-1,3,4-oxadiazol-2-yl)ethyl)carbamate I101

A suspension of tert-butyl (3-oxo-2-phenyl-3-(2-(3,3,3-trifluoropropanoyl) hydrazinyl)propyl)carbamate I100 (312 mg, 0.8 mmol) and Burgess reagent (760 mg, 3.2 mmol) in dry THF (12 mL) was stirred at 160° C. in a sealed tube overnight. The mixture was diluted with DCM (100 mL), dried over Na₂SO₄, filtered and concentrated under reduced pressure. The residue was purified by prep. TLC (DCM/MeOH=20/1) to give the title compound (50 mg, 17%) as a yellow solid. LCMS-D: Rₜ 2.30 min, m/z 372.1 [M+H]⁺.

### c) 2-Phenyl-2-(5-(2,2,2-trifluoroethyl)-1,3,4-oxadiazol-2-yl)ethanamine I102

To a solution of tert-butyl (2-phenyl-2-(5-(2,2,2-trifluoroethyl)-1,3,4-oxadiazol-2-yl) ethyl)carbamate I101 (50 mg, 0.13 mmol) in DCM (10 mL) was added TFA (1 mL) and the mixture was stirred at RT overnight. The mixture was diluted with DCM (50 mL), washed with a saturated aqueous NaHCO₃ solution and concentrated under reduced pressure to give the title compound (20 mg, 60%) as a yellow solid. LCMS-D: Rₜ 0.25 min, m/z 272.0 [M+H]⁺.

### xxxv) 2-(2-Iodophenyl)-2-(oxazol-2-yl)ethanamine (I106)

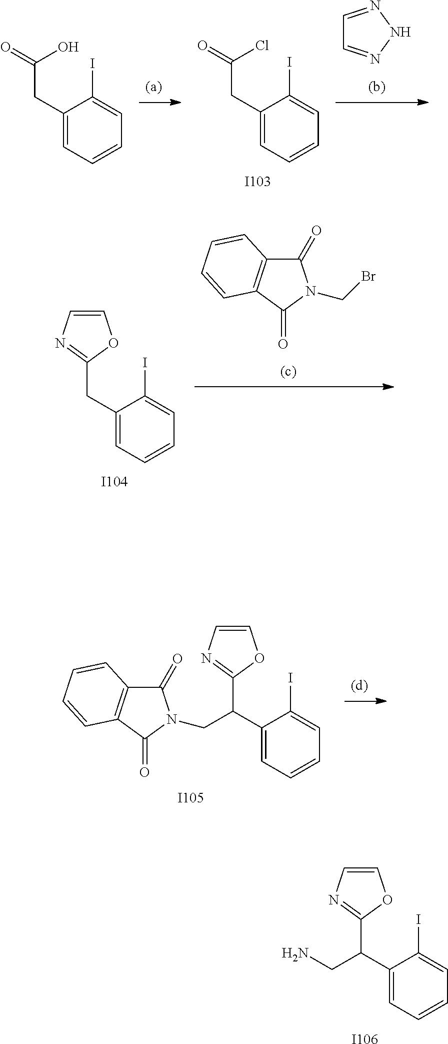

### a) 2-(2-Iodophenyl)acetyl Chloride I103

To a solution of 2-(2-iodophenyl)acetic acid (15.7 g, 60 mmol) and DMF (3 drops) in DCM (100 mL) at 0° C. under N₂ was added oxalyl chloride (23 g, 180 mmol) dropwise and the mixture was stirred for 3 h. The mixture was concentrated under reduced pressure to give the title compound (16.8 g, 100%) as a brown oil. LCMS-D: Rₜ 2.14 min, m/z 276.9 [M−Cl+MeO+H]⁺.

### b) 2-(2-Iodobenzyl)oxazole I104

To a mixture of 1,2,3-triazole (5.0 g, 72.0 mmol) and K₂CO₃ (13.0 g, 90.0 mmol) in sulfolane (200 mL) at 0° C. was added 2-(2-iodophenyl)acetyl chloride I103 (16.8 g, 60.0 mmol) and the mixture was heated at 165° C. for 45 min. After cooling to RT, the mixture was diluted with water, extracted with MTBE (500 mL×3) and the combined organic extracts were dried over Na₂SO₄, filtered and concentrated under reduced pressure. The residue was purified by silica gel chromatography (Pet. ether/EtOAc=20/1) to give the title compound (9.5 g, 55%) as a yellow oil. LCMS-D: Rₜ 1.98 min, m/z 285.9 [M+H]⁺. ¹H NMR (400 MHz, DMSO-d₆) δ 8.00 (d, J=1.0 Hz, 1H), 7.87 (dd, J=7.8, 1.3 Hz, 1H), 7.41-7.32 (m, 2H), 7.12 (d, J=0.9 Hz, 1H), 7.07-7.00 (m, 1H), 4.23 (s, 2H).

### c) 2-(2-(2-Iodophenyl)-2-(oxazol-2-yl)ethyl)isoindoline-1,3-dione I105

To a solution of 2-(2-iodobenzyl)oxazole I104 (9.1 g, 32 mmol) in dry THF (100 mL) at −78° C. under N₂ was added LiHMDS (1 M solution in THF, 38.4 mL, 38.4 mmol) dropwise. The mixture was stirred at −78° C. for 45 min, then added to a solution of 2-(bromomethyl)isoindoline-1,3-dione (9.2 g, 38.4 mmol) in dry THF (150 mL) and the mixture was stirred at −78° C. under N₂ overnight. The mixture was diluted with water, extracted with EtOAc and the organic layer was dried over Na₂SO₄, filtered and concentrated under reduced pressure. The residue was purified by silica gel chromatography (Pet. ether/EtOAc=2/1) to give the title compound (4.6 g, 32%) as a yellow solid. LCMS-D: Rₜ 2.33 min, m/z 444.9 [M+H]⁺.

### d) 2-(2-Iodophenyl)-2-(oxazol-2-yl)ethanamine I106

A suspension of 2-(2-(2-iodophenyl)-2-(oxazol-2-yl)ethyl)isoindoline-1,3-dione 1105 (4.6 g, 11.0 mmol) and hydrazine monohydrate (1.7 g, 33 mmol) in EtOH (120 mL) was heated at 80° C. under N₂ for 3 h. The mixture was filtered and the filtrate was concentrated under reduced pressure to give the title compound (2.7 g, 79%) as an orange oil. LCMS-D: Rₜ 0.28 min, m/z 314.9 [M+H]⁺. ¹H NMR (400 MHz, DMSO-d₆) δ 8.03 (d, J=1.0 Hz, 1H), 7.89 (dd, J=8.0, 1.4 Hz, 1H), 7.38-7.31 (m, 1H), 7.21 (s, 1H), 7.11 (dd, J=7.8, 1.7 Hz, 1H), 7.05-6.98 (m, 1H), 4.52-4.44 (m, 1H), 3.25-3.15 (m, 1H), 3.05-2.97 (m, 1H).

### xxxvi) (2-(2-Amino-1-(oxazol-2-yl)ethyl)phenyl)methanol trifluoroacetate Salt (I110)

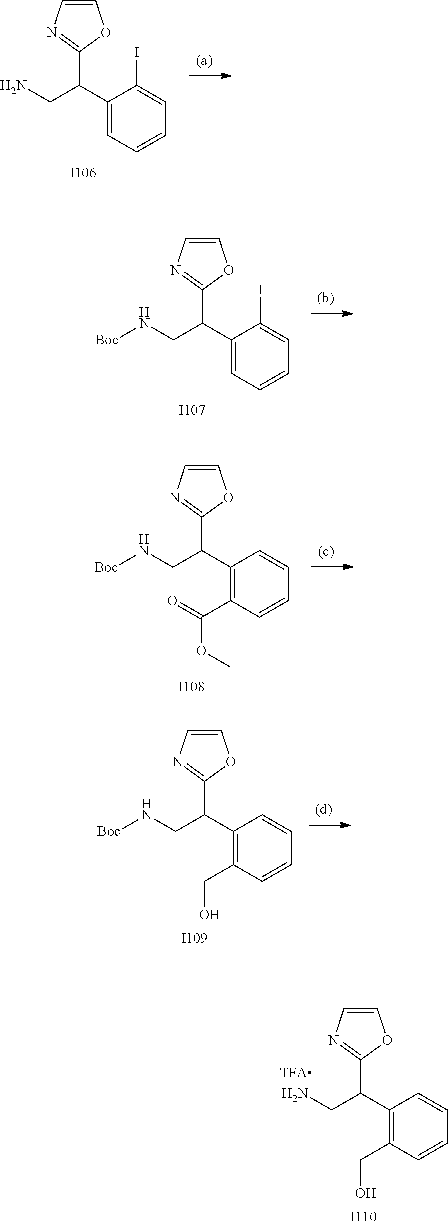

### a) tert-Butyl (2-(2-iodophenyl)-2-(oxazol-2-yl)ethyl)carbamate I107

A suspension of 2-(2-iodophenyl)-2-(oxazol-2-yl)ethanamine I106 (628 mg, 2.0 mmol), Boc₂O (873 mg, 4.0 mmol) and Et₃N (606 mg, 6.0 mmol) in DCM (20 mL) was stirred at RT for 3 h. The mixture was diluted with water, extracted with DCM (100 mL) and the organic layer was dried over Na₂SO₄, filtered and concentrated under reduced pressure. The residue was purified by silica gel chromatography (Pet. ether/EtOAc=4/1) to give the title compound (700 mg, 84%) as a yellow oil. LCMS-C: Rₜ 2.31 min, m/z 414.9 [M+H]⁺.

### b) Methyl 2-(2-((tert-butoxycarbonyl)amino)-1-(oxazol-2-yl)ethyl)benzoate I108

A mixture of tert-butyl (2-(2-iodophenyl)-2-(oxazol-2-yl)ethyl)carbamate I107 (700 mg, 1.7 mmol), Pd(dppf)Cl₂.DCM (140 mg, 0.17 mmol), Et₃N (500 mg, 5 mmol) and MeOH (30 mL) was heated at 100° C. under a CO atmosphere (0.1 MPa) overnight. The mixture was diluted with water, extracted with DCM (100 mL) and the organic layer was dried over Na₂SO₄, filtered and concentrated under reduced pressure to give the title compound (460 mg, 77%) as a yellow oil. LCMS-C: Rₜ 2.19 min, m/z 347.0 [M+H]⁺.

### c) tert-Butyl(2-(2-(hydroxymethyl)phenyl)-2-(oxazol-2-yl)ethyl)carbamate I109

To a solution of methyl 2-(2-((tert-butoxycarbonyl)amino)-1-(oxazol-2-yl)ethyl) benzoate I108 (460 mg, 1.33 mmol) in dry THF (20 mL) was added LiBH₄ (2 M solution in THF, 1.33 mL, 2.66 mmol) and the mixture was stirred at RT for 2 h. The mixture was diluted with DCM (100 mL), washed with water, dried over Na₂SO₄, filtered and concentrated under reduced pressure to give the title compound (400 mg, 98%) as a yellow oil. LCMS-C: Rₜ 1.37 min, m/z 319.0 [M+H]⁺.

### d) (2-(2-Amino-1-(oxazol-2-yl)ethyl)phenyl)methanol Trifluoroacetate Salt I110

A solution of tert-butyl (2-(2-(hydroxymethyl)phenyl)-2-(oxazol-2-yl)ethyl) carbamate I109 (100 mg, 0.3 mmol) in TFA (1 mL) was stirred at RT for 2 h. The mixture was then concentrated under reduced pressure to give the title compound (66 mg, 67%) as a yellow oil. LCMS-C: Rₜ 0.38 min, m/z 219.0 [M+H]⁺.

### xxxvii) 2-Phenyl-2-(thiazol-2-yl)ethanamine (I113)

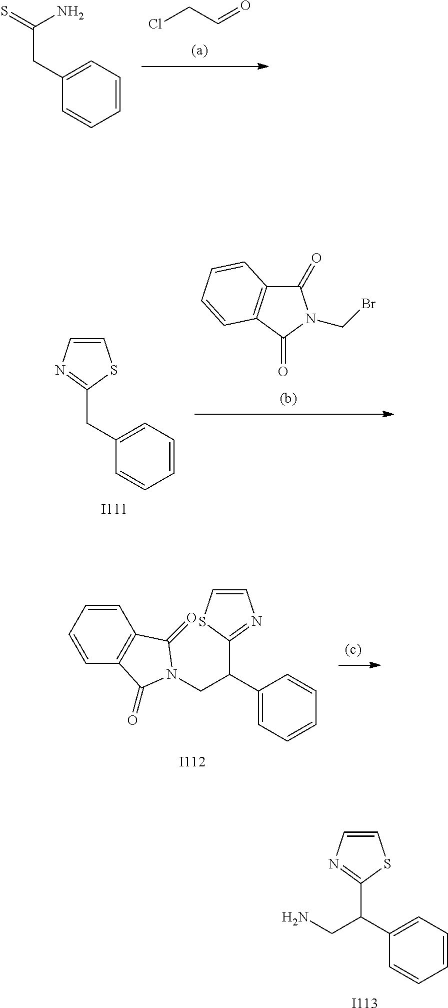

### a) 2-Benzylthiazole I111

A suspension of 2-phenylethanethioamide (10.0 g, 66.0 mmol) and 2-chloroacetaldehyde (26.0 g, 132 mmol) in EtOH (150 mL) was heated at 100° C. under N₂ overnight. The mixture was diluted with EtOAc (500 mL), dried over Na₂SO₄, filtered and concentrated under reduced pressure. The residue was purified by silica gel chromatography (Pet. ether/EtOAc=10/1) to give the title compound (3.88 g, 33%) as a yellow oil. LCMS-C: Rₜ 1.52 min, m/z 176.0 [M+H]⁺.

### b) 2-(2-Phenyl-2-(thiazol-2-yl)ethyl)isoindoline-1,3-dione I112

To a solution of 2-benzylthiazole I111 (3.88 g, 22.1 mmol) in dry THF (60 mL) at −78° C. under N₂ was added LiHMDS (1 M solution in THF, 26.5 mL, 26.5 mmol) dropwise. The mixture was stirred at −78° C. for 45 min, then added to a solution of 2-(bromomethyl)isoindoline-1,3-dione (6.38 g, 26.5 mmol) in dry THF (60 mL) at −78° C. under N₂ and the mixture was stirred at −78° C. overnight. The mixture was diluted with EtOAc (300 mL), washed with water, dried over Na₂SO₄, filtered and concentrated under reduced pressure. The residue was purified by silica gel chromatography (Pet. ether/EtOAc=2/1) to give the title compound (2.9 g, 39%) as a yellow solid. LCMS-C: Rₜ 2.23 min, m/z 335.0 [M+H]⁺.

### c) 2-Phenyl-2-(thiazol-2-yl)ethanamine I113

A suspension of 2-(2-phenyl-2-(thiazol-2-yl)ethyl)isoindoline-1,3-dione I112 (2.9 g, 8.68 mmol) and hydrazine monohydrate (1.3 g, 26.0 mmol) in EtOH (120 mL) was heated at 80° C. under N₂ overnight. The mixture was then filtered and the filtrate was concentrated under reduced pressure to give the title compound (1.4 g, 80%) as a yellow oil. LCMS-C: Rₜ 0.33 min, 205.0 [M+H]⁺.

### xxxviii) 2-(2-(Methoxymethyl)phenyl)-2-(oxazol-2-yl)ethanamine Trifluoroacetate (I115)

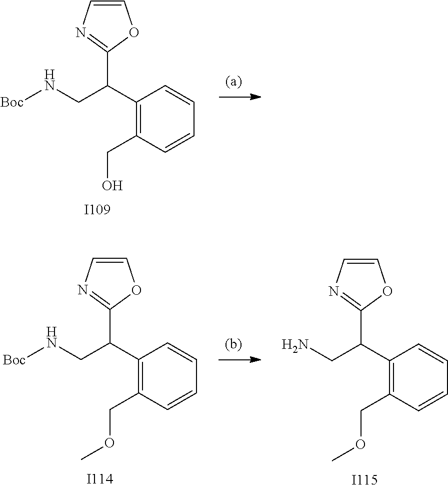 [SMILES: "I109" ; CC(C)(C)OC(=O)NCC(c1ncco1)c1ccccc1CO ; "I114" ; "I115" ; COCc1ccccc1C(CNC(=O)OC(C)(C)C)c1ncco1 ; COCc1ccccc1C(CN)c1ncco1]

### a) tert-Butyl(2-(2-(methoxymethyl)phenyl)-2-(oxazol-2-yl)ethyl)carbamate I114

To a solution of tert-butyl (2-(2-(hydroxymethyl)phenyl)-2-(oxazol-2-yl)ethyl)carbamate I109 (100 mg, 0.30 mmol) in CH₃CN (10 mL) was added Ag₂O (350 mg, 1.5 mmol) and CH₃I (426 mg, 3.0 mmol) and the mixture was stirred at RT overnight. The mixture was diluted with DCM (100 mL), dried over Na₂SO₄, filtered and concentrated under reduced pressure to give the title compound (40 mg, 40%) as a yellow oil. LCMS-C: Rₜ 2.28 min, m/z 333.1 [M+H]⁺.

### b) 2-(2-(Methoxymethyl)phenyl)-2-(oxazol-2-yl)ethanamine trifluoroacetate I115

A solution of tert-butyl(2-(2-(methoxymethyl)phenyl)-2-(oxazol-2-yl)ethyl)carbamate I114 (40 mg, 0.12 mmol) in TFA (1 mL) was stirred at RT for 2 h. The mixture was then concentrated under reduced pressure to give the title compound (23 mg, 56%) as a yellow oil. LCMS-C: Rₜ 0.35 min, m/z 233.0 [M+H]⁺.

### xxxix) 2-Amino-1-cyclohexylethanol Hydrochloride I116

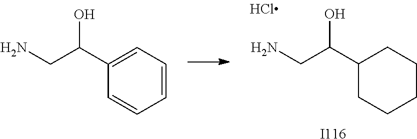 [SMILES: "I116" ; NCC(O)c1ccccc1 ; NCC(O)C1CCCCC1]

To a solution of 2-amino-1-phenylethanol (274 mg, 2.0 mmol) in EtOH (20 mL) was added PtO₂ (45 mg, 0.2 mmol) and conc. aqueous HCl (1 mL) and the mixture was heated at 120° C. under a H₂ atmosphere (3 MPa) overnight. The mixture was filtered and the filtrate was concentrated under reduced pressure to give the title compound (57 mg, 16%) as a yellow oil, which was used directly in the next step without further purification. LCMS-C: Rₜ 0.32 min, m/z 144.1 [M+H]⁺.

### xl) (1-(Pyridin-2-yl)cyclopentyl)methanamine I118

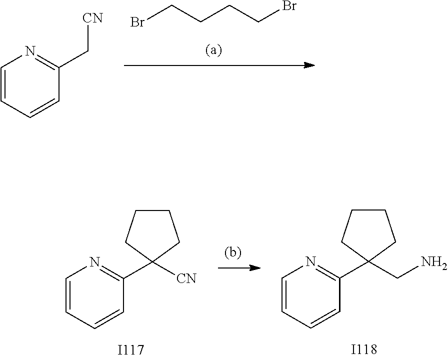 [SMILES: N#CCc1ccccn1 ; BrCCCCBr ; "I117" ; "I118" ; N#CC1(c2ccccn2)CCCC1 ; NCC1(c2ccccn2)CCCC1]

### a) 1-(Pyridin-2-yl)cyclopentanecarbonitrile I117

To a solution of NaH (60% dispersion in mineral oil, 800 mg, 20 mmol) in DMSO (10 mL) at 15° C. under N₂ was added a solution of 2-(pyridin-2-yl)acetonitrile (1.18 g, 10 mmol) and 1,4-dibromobutane (2.16 g, 10 mmol) in Et₂O (10 mL) and DMSO (2 mL) dropwise over 1 h. The mixture was then allowed to warm to RT and stirred for 24 h. The reaction was carefully quenched by dropwise addition of isopropanol (5 mL) followed by water (10 mL). The mixture was stirred for 10 min, then extracted with EtOAc (200 mL) and the organic layer was washed with water, dried over Na₂SO₄, filtered and concentrated under reduced pressure to give the title compound (1.72 g, 100%) as a brown oil. LCMS-C: Rₜ 1.11 min, m/z 173.0 [M+H]⁺.

### b) (1-(Pyridin-2-yl)cyclopentyl)methanamine I118

To a solution of 1-(pyridin-2-yl)cyclopentanecarbonitrile I117 (344 mg, 2 mmol) in THF (10 mL) was added LiAlH₄ (2.5 M solution in THF, 1.6 mL, 4 mmol) and the mixture was stirred at RT for 2 h. The mixture was diluted with water (5 mL), extracted with EtOAc (100 mL) and the organic extract was dried over Na₂SO₄, filtered and concentrated under reduced pressure to give the title compound (200 mg, 60%) as a yellow oil. LCMS-C: Rₜ 0.33 min, m/z 177.1 [M+H]⁺.

### xli) (1-(Pyridin-2-yl)cyclohexyl)methanamine (I120)

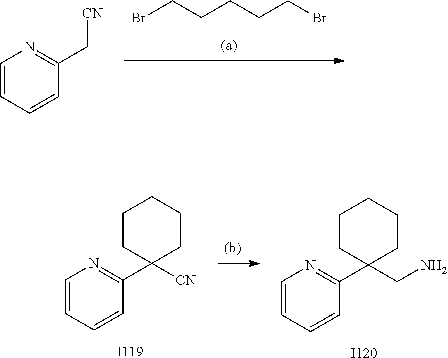 [SMILES: N#CCc1ccccn1 ; BrCCCCCBr ; "I119" ; "I120" ; N#CC1(c2ccccn2)CCCCC1 ; NCC1(c2ccccn2)CCCCC1]

### a) 1-(Pyridin-2-yl)cyclohexanecarbonitrile I119

To a solution of NaH (60% dispersion in mineral oil, 800 mg, 20 mmol) in DMSO (10 mL) at 15° C. under N₂ was added a solution of 2-(pyridin-2-yl)acetonitrile (1.18 g, 10 mmol) and 1,5-dibromopentane (2.3 g, 10 mmol) in Et₂O (80 mL) and DMSO (2 mL) dropwise over 1 h. The mixture was allowed to warm to RT and stirred for 24 h. The reaction was carefully quenched by dropwise addition of isopropanol (5 mL) followed by water (10 mL). The mixture was stirred for 10 min, then extracted with EtOAc (200 mL) and the organic layer was washed with water, dried over Na₂SO₄, filtered and concentrated under reduced pressure to give the title compound (1.86 g, 100%) as a brown oil. LCMS-C: Rₜ 1.87 min, m/z 187.0 [M+H]⁺.

### b) (1-(Pyridin-2-yl)cyclohexyl)methanamine I120

To a solution of 1-(pyridin-2-yl)cyclohexanecarbonitrile I119 (372 mg, 2 mmol) in THF (10 mL) was added LiAlH₄ (2.5 M solution in THF, 1.6 mL, 4 mmol) and the mixture was stirred at RT for 2 h. The mixture was diluted with water (5 mL), extracted with EtOAc (100 mL) and the organic extract was dried over Na₂SO₄, filtered and concentrated under reduced pressure to give the title compound (240 mg, 60%) as a yellow oil. LCMS-C: Rₜ 0.35 min, m/z 191.1 [M+H]⁺.

### xlii) 2-Phenyl-2-(pyridin-2-yl)ethanamine (I121)

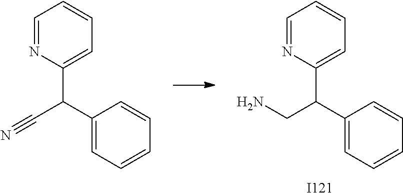 [SMILES: "I121" ; N#CC(c1ccccc1)c1ccccn1 ; NCC(c1ccccc1)c1ccccn1]

A mixture of 2-phenyl-2-(pyridin-2-yl)acetonitrile (100 mg, 0.5 mmol) and Raney nickel (20 mg) in conc. aqueous NH₄OH (2 mL) was heated at 50° C. under a H₂ atmosphere overnight. The mixture was then filtered and the filtrate was partitioned between EtOAc and water. The layers were separated and the organic layer was dried over Na₂SO₄, filtered and concentrated under reduced pressure to give the title compound (50 mg, 49%). LCMS-C: Rₜ 0.36 min, m/z 199.1 [M+H]⁺

### xliii) 2-(4-Fluorophenyl)-2-(oxazol-2-yl)ethanamine I124

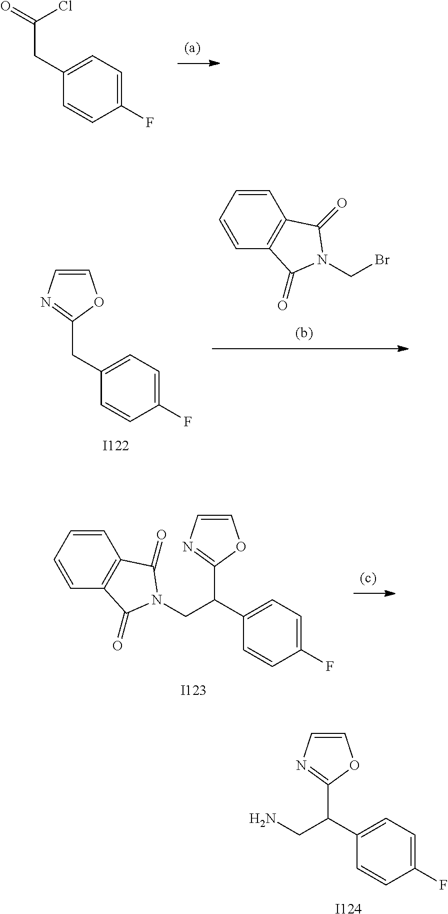

### a) 2-(4-Fluorobenzyl)oxazole I122

To a mixture of 1,2,3-triazole (10 g, 0.14 mol) and K₂CO₃ (25 g, 0.18 mmol) in sulfolane (300 mL) at 0° C. was added 2-(4-fluorophenyl)acetyl chloride (20 g, 0.12 mol) dropwise and the mixture was heated at 165° C. for 1 h. After cooling to RT, the mixture was diluted with MTBE (500 mL), washed with brine, then dried over Na₂SO₄, filtered and concentrated under reduced pressure. The residue was purified by silica gel chromatography (Pet. ether/EtOAc=20/1) to give the title compound (10.5 g, 51%) as a red solid. LCMS-D: Rₜ 1.40 min; m/z 178.0 [M+H]⁺.

### b) 2-(2-(4-Fluorophenyl)-2-(oxazol-2-yl)ethyl)isoindoline-1,3-dione I123

To a solution of 2-(4-fluorobenzyl)oxazole I122 (10 g, 56 mmol) in THF (200 mL) at −78° C. under N₂ was LiHMDS (1 M solution in THF, 67.2 mL, 67.2 mmol) dropwise. The mixture was stirred for 45 min at −78° C., then added dropwise to a solution of 2-(bromomethyl)isoindoline-1,3-dione (16.1 g, 67.2 mmol) in THF (200 mL) at −78° C. and the mixture was stirred at −78° C. overnight. The mixture was diluted with water, extracted with EtOAc (500 mL×3) and the combined organic extracts were dried over Na₂SO, filtered and concentrated under reduced pressure. The residue was purified by silica gel chromatography (Pet. ether/EtOAc=8/1 to 4/1) to give the title compound (3.0 g, 16%) as a white solid, which was used directly in the next step.

### c) 2-(4-Fluorophenyl)-2-(oxazol-2-yl)ethanamine I124

A suspension of 2-(2-(4-fluorophenyl)-2-(oxazol-2-yl)ethyl)isoindoline-1,3-dione I123 (1.0 g, 3.0 mmol) and hydrazine monohydrate (451 mg, 9.0 mmol) in EtOH (50 mL) was heated at 80° C. for 3 h. The mixture was filtered and the solid was washed with EtOH (50 mL). The filtrate was then concentrated under reduced pressure to give the title compound (532 mg, 87%) as a yellow oil. LCMS-C: Rₜ 0.29 min; m/z 207.0 [M+H]⁺.

### xliv) 2-(3-Chlorophenyl)-2-(oxazol-2-yl)ethanamine (I128)

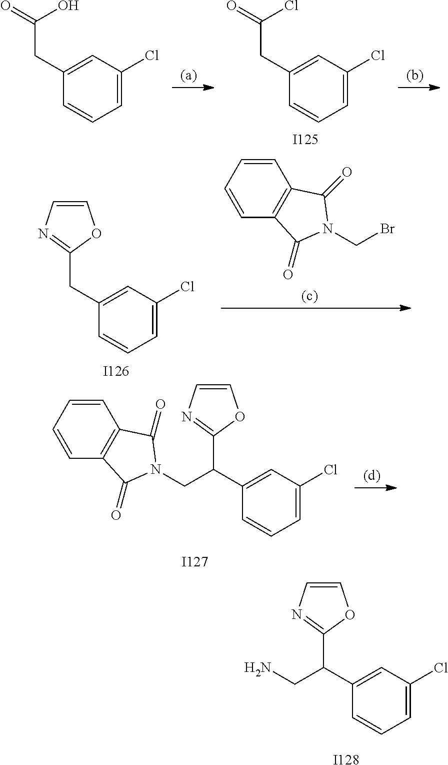

### a) 2-(3-Chlorophenyl)acetyl chloride I125

To a solution of 2-(3-chlorophenyl)acetic acid (20.0 g, 0.12 mol) and DMF (0.2 mL) in DCM (100 mL) was added oxalyl chloride (45.7 g, 0.36 mol) dropwise and the mixture was stirred at RT for 1 h. The mixture was then concentrated under reduced pressure to give the title compound (10.0 g, 45%) as a red oil. LCMS-C: Rₜ 2.03 min; m/z 185.0 [M−Cl+MeO+H]⁺.

### b) 2-(3-Chlorobenzyl)oxazole I126

To a mixture of 1,2,3-triazole (8.8 g, 0.13 mol) and K₂CO₃ (23.5 g, 0.17 mol) in sulfolane (300 mL) at 0° C. was added 2-(3-chlorophenyl)acetyl chloride I125 (20.0 g, 0.11 mol) dropwise and the mixture was heated at 165° C. for 1 h. After cooling to RT, the mixture was diluted with MTBE (500 mL) and washed with brine, dried over Na₂SO₄, filtered and concentrated under reduced pressure. The residue was purified by silica gel chromatography (Pet. ether/EtOAc=10/1) to give the title compound (10.7 g, 53%) as a yellow oil. LCMS-C: Rₜ 1.96 min; m/z 194.0 [M+H]⁺.

### c) 2-(2-(3-Chlorophenyl)-2-(oxazol-2-yl)ethyl)isoindoline-1,3-dione I127

To a solution of 2-(3-chlorobenzyl)oxazole I126 (10.0 g, 51.6 mmol) in dry THF (200 mL) at −78° C. under N₂ was added LiHMDS (1 M solution in THF, 62.0 mL, 62.0 mmol). The mixture was stirred at −78° C. for 45 min, then added to a solution of 2-(bromomethyl)isoindoline-1,3-dione (14.9 g, 62.0 mmol) in THF (200 mL) at −78° C. and the mixture was stirred at −78° C. overnight. The mixture was diluted with water and extracted with EtOAc (500 mL×3). The combined organic extracts were dried over Na₂SO₄, filtered and concentrated under reduced pressure. The residue was purified by silica gel chromatography (Pet. ether/EtOAc=8/1 to 4/1) to give the title compound (6.8 g, 37%) as a white solid. LCMS-C: Rₜ 2.31 min; m/z 352.9 [M+H]⁺.

### d) 2-(3-Chlorophenyl)-2-(oxazol-2-yl)ethanamine I128

A suspension of 2-(2-(3-chlorophenyl)-2-(oxazol-2-yl)ethyl)isoindoline-1,3-dione I127 (1.0 g, 2.8 mmol) and hydrazine monohydrate (426 mg, 8.5 mmol) in EtOH (50 mL) was heated at 80° C. for 3 h. The mixture was then filtered and the solid was washed with EtOH (50 mL). The filtrate was concentrated under reduced pressure to give the title compound (0.56 g, 89%) as a yellow oil. LCMS-C: Rₜ 0.31 min; m/z 223.0 [M+H]⁺.

### xlv) 5-(2-(2-Aminoethyl)phenyl)-3-methyl-1,3,4-oxadiazol-2(3H)-one Trifluoroacetate (I131)

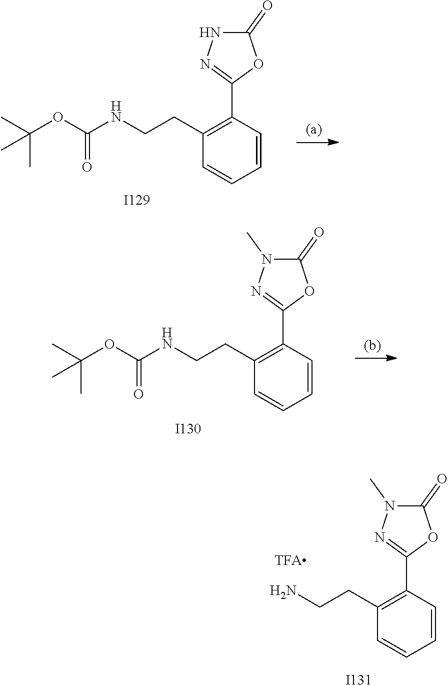 [SMILES: "I129" ; "I130" ; CC(C)(C)OC(=O)NCCc1ccccc1-c1n[nH]c(=O)o1 ; Cn1nc(-c2ccccc2CCNC(=O)OC(C)(C)C)oc1=O ; "I131" ; Cn1nc(-c2ccccc2CCN)oc1=O]

### a) tert-Butyl 2-(4-methyl-5-oxo-4,5-dihydro-1,3,4-oxadiazol-2-yl)phenethylcarbamate I130

A mixture of tert-butyl 2-(5-oxo-4,5-dihydro-1,3,4-oxadiazol-2-yl)phenethylcarbamate I129 (see below) (200 mg, 0.66 mmol), K₂CO₃ (181 mg, 1.31 mmol) and CH₃I (186 mg, 1.31 mmol) in DMF (10 mL) was stirred at RT under N₂ overnight. Water was added and the mixture was extracted with EtOAc. The organic extract was dried over Na₂SO₄, filtered and concentrated under reduced pressure to give the title compound (389 mg, >100%) as a yellow oil, which was used directly in the next step. LCMS-C: Rₜ 2.17 min; m/z 342.0 [M+Na]⁺.

### b) 5-(2-(2-Aminoethyl)phenyl)-3-methyl-1,3,4-oxadiazol-2(3H)-one Trifluoroacetate I131

A mixture of tert-butyl 2-(4-methyl-5-oxo-4,5-dihydro-1,3,4-oxadiazol-2-yl)phenethylcarbamate I130 (389 mg, assumed 0.66 mmol) and TFA (5 mL) in DCM (10 mL) was stirred at RT under N₂ overnight. The mixture was concentrated under reduced pressure to give the title product (210 mg, 95%) as a yellow oil. LCMS-C: Rₜ 0.34 min; m/z 220.0 [M+H]⁺.

### xlvi) N-Methyl-2-(oxazol-2-yl)-2-phenylethan-1-amine (I133)

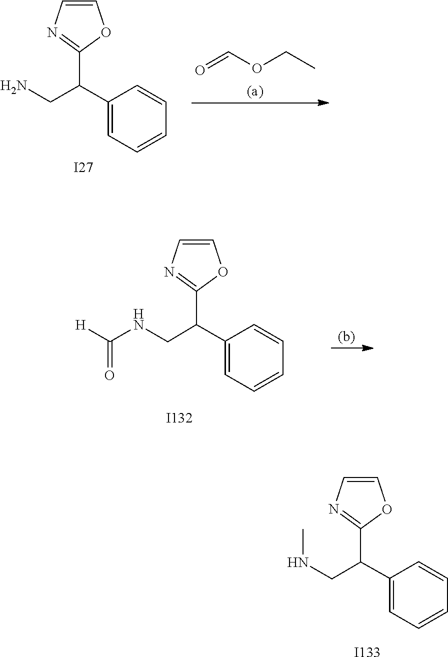

### a) N-(2-(Oxazol-2-yl)-2-phenylethyl)formamide I132

A solution of 2-(oxazol-2-yl)-2-phenylethan-1-amine 127 (600 mg, 3.19 mmol) in ethyl formate (15 mL) was heated at 80° C. for 3 h. After cooling to RT, water (50 mL) was added and the mixture was extracted with DCM (50 mL×3). The combined organic extracts were dried over Na₂SO₄, filtered and concentrated under reduced pressure to give the title compound (500 mg, 72%), which was used directly in the next step without further purification. LCMS-D: Rₜ 0.46 min; m/z 217.1 [M+H]⁺.

### b) N-Methyl-2-(oxazol-2-yl)-2-phenylethan-1-amine I133

A mixture of N-(2-(oxazol-2-yl)-2-phenylethyl)formamide I132 (300 mg, 1.39 mmol) and BH₃.THF (1 M solution in THF, 6 mL, 6 mmol) was heated at 70° C. for 3 h, then allowed to cool to RT, adjusted to pH 5 with 10% aqueous HCl and stirred for 1 h. The mixture was washed with EtOAc (40 mL×3) and the aqueous layer was then adjusted pH 9 with 1 M aqueous NaOH and extracted with EtOAc (40 mL×3). The combined organic extracts were dried over Na₂SO₄, filtered and concentrated under reduced pressure to give the title compound (130 mg, 46%) as a yellow oil. LCMS-D: Rₜ 0.32 min; m/z 203.1 [M+H]⁺.

### xlvii) 2-(2-(1H-Imidazol-1-yl)phenyl)ethan-1-amine dihydrochloride (I135)

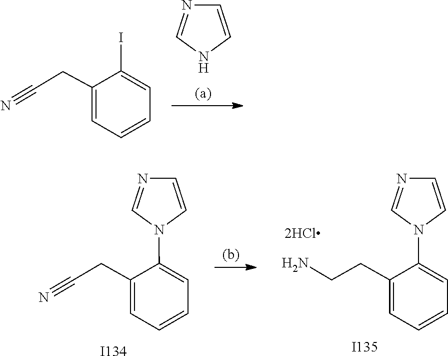

### a) 2-(2-(1H-Imidazol-1-yl)phenyl)acetonitrile I134

A mixture of 2-(2-iodophenyl)acetonitrile (600 mg, 2.47 mmol), 1H-imidazole (252 mg, 3.7 mmol), Fe(acac)₃ (262 mg, 0.741 mmol), Cs₂CO₃ (1.61 g, 4.94 mmol) and CuO (20 mg, 0.247 mmol) in DMF (15 mL) was heated at 90° C. under N₂ in a sealed tube for 30 h. The mixture was then filtered and the filtrate was diluted with water (30 mL) and extracted with EtOAc (30 mL×3). The combined organic extracts were concentrated under reduced pressure and the residue was purified by silica gel chromatography (DCM/MeOH=15/1) to give the title compound (180 mg, 40%) as a yellow oil. LCMS-D: Rₜ 2.43 min, m/z 184.0 [M+H]⁺.

### b) 2-(2-(1H-Imidazol-1-yl)phenyl)ethan-1-amine Dihydrochloride I135

To a solution of 2-(2-(1H-imidazol-1-yl)phenyl)acetonitrile I134 (90 mg, 0.49 mmol) in MeOH (5 mL) was added 10% Pd/C (50 mg) and conc. aqueous HCl (0.2 mL) and the mixture was stirred at RT under a H₂ atmosphere overnight. The mixture was filtered and the filter cake rinsed with MeOH (3 mL×2). The filtrate was concentrated under reduced pressure to give the title compound (80 mg, 63%) as a yellow oil. LCMS-D: Rₜ 0.89 min, m/z 188.0 [M+H]⁺.

### xlviii) 2-([1,1′-Biphenyl]-2-yl)-2-(oxazol-2-yl)ethanamine (I136)

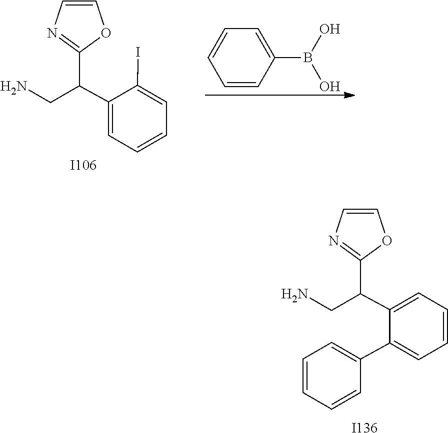 [SMILES: "I106" ; NCC(c1ncco1)c1ccccc1I ; OB(O)c1ccccc1 ; "I136" ; NCC(c1ncco1)c1ccccc1-c1ccccc1]

To a solution of 2-(2-iodophenyl)-2-(oxazol-2-yl)ethanamine I106 (157 mg, 0.5 mmol) in DMF/H₂O (10 mL/2 mL) was added phenylboronic acid (122 mg, 1 mmol), Pd(PPh₃)₄ (57 mg, 0.05 mmol) and Cs₂CO₃ (450 mg, 1.5 mmol) and the mixture was heated at 110° C. under N₂ overnight. The mixture was diluted with EtOAc (100 mL), washed with water (100 mL×5) and the organic layer was dried over Na₂SO₄, filtered and concentrated under reduced pressure. The residue was purified by prep. TLC (DCM/MeOH=20/1) to give the title compound (20 mg, 15%) as a yellow oil. LCMS-C: Rₜ 0.55 min, m/z 265.0 [M+H]⁺.

### xlix) 5-(2-(2-Aminoethyl)phenyl)-1,3,4-oxadiazol-2(3H)-one (I141)

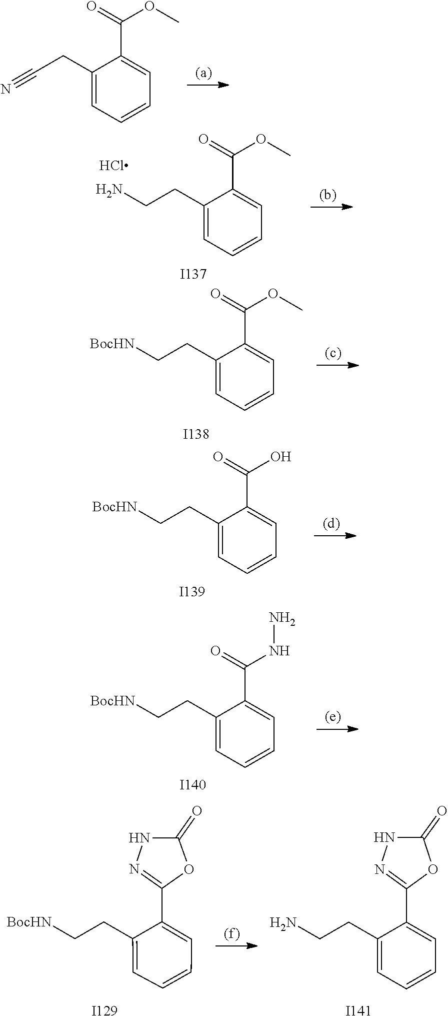

### a) Methyl 2-(2-aminoethyl) Benzoate Hydrochloride I137

To a solution of methyl 2-(cyanomethyl) benzoate (2.09 g, 11.9 mmol) in MeOH (30 mL) was added 10% Pd/C (1.05 g) and conc. aqueous HCl (5 mL) and the mixture was stirred at RT under a H₂ atmosphere overnight. The mixture was filtered and the filtrate was concentrated under reduced pressure. The residue was suspended in MeOH (5 mL) then diluted with Et₂O (100 mL). The solid was collected by filtration, washed with Et₂O and dried under vacuum to give the title compound (1.25 g 58%) as a white solid. LCMS-D: Rₜ 0.31 min; m/z 180.1 [M+H]⁺.

### b) Methyl 2-(2-((tert-butoxycarbonyl)amino)ethyl)benzoate I138

A solution of methyl 2-(2-aminoethyl) benzoate I137 (1.22 g 6.82 mmol), Boc₂O (2.23 g, 10.2 mmol) and Et₃N (2.07 g, 20.5 mmol) in DCM (30 mL) was stirred at RT under N₂ overnight. The mixture was partitioned between water and EtOAc, the layers were separated and the organic layer was washed with water, brine, dried over Na₂SO₄, filtered and concentrated under reduced pressure to give the title compound (1.87 g, 98%) as a yellow oil. LCMS-D: Rₜ 2.27 min; m/z 180.1 [M-Boc+2H]⁺.

### c) 2-(2-((tert-Butoxycarbonyl)amino)ethyl)benzoic Acid I139

To a solution of methyl 2-(2-((tert-butoxycarbonyl)amino)ethyl)benzoate I138 (1.87 g, 6.72 mmol) in MeOH (18 mL) and water (5 mL) was added NaOH (1.34 g, 33.6 mmol) and the mixture was heated at 50° C. for 5 h. The mixture was partitioned between water and EtOAc, the layers were separated and the organic layer was extracted with water. The combined aqueous layers were acidified to pH 2 with 1 M aqueous HCl and extracted with EtOAc. The organic extract was then dried over Na₂SO₄, filtered and concentrated under reduced pressure to give the title compound (986 mg 55%) as a yellow solid. LCMS (ES-API): Rₜ 1.83 min; m/z 264.1 [M−H]⁻.

### d) tert-Butyl (2-(hydrazinecarbonyl) phenethyl)carbamate I140

To a solution of 2-(2-((tert-butoxycarbonyl)amino)ethyl)benzoic acid I139 (980 mg, 3.70 mmol) in THF (15 mL) was added CDI (719 mg, 4.44 mmol) and the mixture was stirred at RT for 2 h. Hydrazine monohydrate (555 mg, 11.1 mmol) was then added and the mixture was stirred at RT for a further 5 h. The mixture was partitioned between water and EtOAc, the layers were separated and the organic layer was dried over Na₂SO₄, filtered and concentrated under reduced pressure to give the title compound (1.00 g, 99%) as a colorless oil. LCMS-D: Rₜ 0.48 min; m/z 180.1 [M-Boc+2H]⁺.

### e) tert-Butyl (2-(5-oxo-4,5-dihydro-1,3,4-oxadiazol-2-yl)phenethyl)carbamate I129

To a solution of tert-butyl (2-(hydrazinecarbonyl) phenethyl)carbamate I140 (1.00 g, 3.69 mmol) in THF (20 mL) was added CDI (1.79 g, 11.1 mmol) and the mixture was heated at reflux for 6 h. The solvent was removed under reduced pressure and the residue was diluted with water. The resulting precipitate was collected by filtration, washed with water and dried under vacuum to give the title compound (900 mg, 80%) as a yellow oil. LCMS-D: Rₜ 1.91 min; m/z 206.0 [M-Boc+2H]⁺.

### f) 5-(2-(2-Aminoethyl)phenyl)-1,3,4-oxadiazol-2(3H)-one I141

A mixture of tert-butyl (2-(5-oxo-4,5-dihydro-1,3,4-oxadiazol-2-yl)phenethyl)carbamate I129 (850 mg, 2.79 mmol) and TFA (8 mL) in DCM (2 mL) was stirred at RT for 5 h. The mixture was concentrated under reduced pressure and the residue was purified by silica gel chromatography (DCM/MeOH=50/1 to 30/1) to give the title compound (380 mg, 66%) as a white solid. LCMS-D: Rₜ 0.31 min; m/z 206.1 [M+H]⁺.

### I) 2-(Oxazol-2-yl)-2-(p-tolyl)ethan-1-amine (I145)

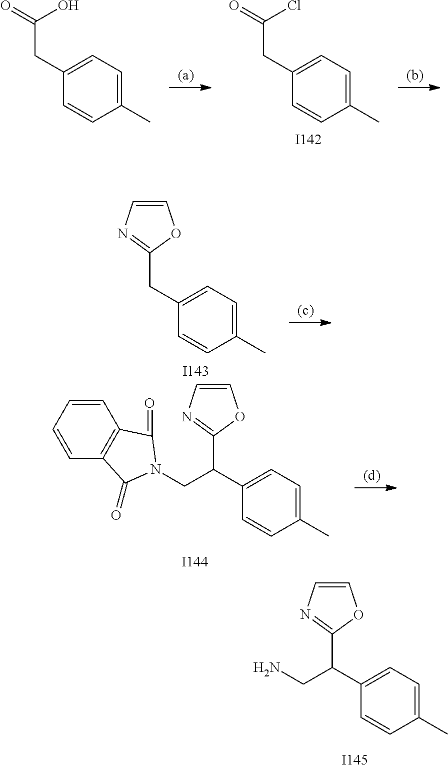

### a) 2-(p-Tolyl)acetyl Chloride I142

To a solution of 2-(p-tolyl) acetic acid (12.7 g, 84.6 mmol) and DMF (0.2 mL) in DCM (100 mL) was added oxalyl chloride (32.2 g, 254 mmol) dropwise and the mixture was stirred at RT for 1 h. The mixture was then concentrated under reduced pressure to give the title compound (10.1 g, 71%), which was used directly in the next step. LCMS-C: Rₜ 2.00 min; m/z 165.0 [M−Cl+MeO+H]⁺.

### b) 2-(4-Methylbenzyl)oxazole I143

To a solution of 1,2,3-1H-triazole (4.9 g, 71.2 mmol) and K₂CO₃ (12.3 g, 88.9 mmol) in sulfolane (150 mL) at RT was added 2-(p-tolyl)acetyl chloride I142 (10.0 g, 59.3 mmol) dropwise and the mixture was heated at 165° C. under N₂ for 1 h. After cooling to RT, the mixture was diluted with water (200 mL) and extracted with diethyl ether (200 mL×3). The combined organic extracts were washed with water, dried over Na₂SO₄, filtered and concentrated under reduced pressure. The residue was purified by silica gel chromatography (Pet. ether/EtOAc=20/1 to 15/1) to give the title compound (7.2 g, 70%) as a burgundy colored oil. LCMS-C: Rₜ 1.77 min; m/z 174.0 [M+H]⁺.

### c) 2-(2-(Oxazol-2-yl)-2-(p-tolyl)ethyl)isoindoline-1,3-dione I144

To a solution of 2-(4-methylbenzyl)oxazole I143 (7.0 g, 40.5 mmol) in anhydrous THF (200 mL) at −78° C. under N₂ was added LiHMDS (1 M solution in THF, 49.0 mL, 49.0 mmol) dropwise. The mixture was stirred at −78° C. for 1 h then added to a solution of 2-(bromomethyl)isoindoline-1,3-dione (11.7 g, 48.6 mmol) in anhydrous THF (100 mL) dropwise. The mixture was then allowed to warm to RT and stirred overnight. The reaction was quenched with a saturated aqueous NH₄Cl solution (50 mL) and the mixture was diluted with water (500 mL) and extracted with EtOAc (500 mL×3). The combined organic extracts were washed with brine, dried over Na₂SO₄, filtered and concentrated under reduced pressure. The residue was purified by silica gel chromatography (Pet. ether/EtOAc=20/1 to 5/1) to give the title compound (3.5 g, 26%) as a yellow oil. LCMS-C: Rₜ 2.22 min; m/z 333.0 [M+H]⁺.

### d) 2-(Oxazol-2-yl)-2-(p-tolyl)ethan-1-amine I145

A mixture of 2-(2-(oxazol-2-yl)-2-(p-tolyl)ethyl)isoindoline-1,3-dione I144 (3.5 g, 10.5 mmol) and hydrazine monohydrate (1.58 g, 31.6 mmol) in EtOH (120 mL) was heated at 80° C. for 3 h. The mixture was then filtered and the filtrate was concentrated under reduced pressure to give the title compound (1.5 g, 70%) as a yellow oil. LCMS-C: Rₜ 0.38 min; m/z 203.0 [M+H]⁺. ¹H NMR (400 MHz, DMSO-d₆) δ 7.99 (d, J=0.8 Hz, 1H), 7.16 (d, J=0.7 Hz, 1H), 7.14-7.08 (m, 4H), 4.13 (m, 1H), 3.21 (m, 1H), 2.98 (m, 1H), 2.26 (s, 3H).

### li) 2-(3-Methoxy-5-methylphenyl)-2-(oxazol-2-yl)ethan-1-amine (I150)

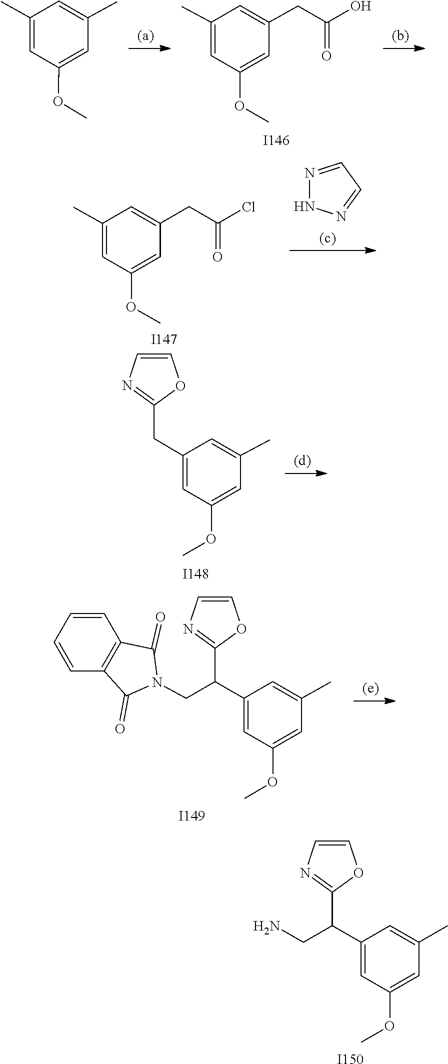

### a) 2-(3-Methoxy-5-methylphenyl)acetic Acid I146

To a solution of 1-methoxy-3,5-dimethylbenzene (10.0 g, 73.4 mmol) in THF (400 mL) at −78° C. was added n-BuLi (2.5 M solution in hexane, 38.0 mL, 95.5 mmol) dropwise and the mixture was stirred for 15 min. t-BuOK (1 M solution in THF, 88.0 mL, 88.0 mmol) was then added dropwise followed by 2,2,6,6-tetramethylpiperidine (10.4 g, 73.4 mmol) and the mixture was stirred at −78° C. for 30 min. The reaction was quenched with excess dry ice and the mixture was allowed to RT. The solvent was removed under reduced pressure and the residue was diluted with Et₂O (500 mL×4) and extracted with 2 M aqueous NaOH (3×50 mL). The combined aqueous layers were acidified to pH 1 with 2 M aqueous HCl, extracted with DCM and the organic extract was dried over Na₂SO₄, filtered and concentrated under reduced pressure to give the title compound (10.0 g, 75%) as a brown oil. LCMS-C: Rₜ 0.79 min; m/z 181.0 [M+H]⁺.

### b) 2-(3-Methoxy-5-methylphenyl)acetyl Chloride I147

To a solution of 2-(3-methoxy-5-methylphenyl)acetic acid I146 (1.7 g, 9.5 mmol) in DCM (100 mL) was added oxalyl chloride (3.62 g, 28.5 mmol) dropwise and DMF (1 mL) and the mixture was stirred at RT for 3 h. The mixture was then concentrated under reduced pressure to give the title compound (1.63 g, 86%) as a red solid, which was used directly in the next step.

### c) 2-(3-Methoxy-5-methylbenzyl)oxazole I148

To a solution of 1,2,3-1H-triazole (679 mg, 9.84 mmol) and K₂OO₃(1.70 g, 12.3 mmol) in sulfolane (300 mL) at RT was added 2-(3-methoxy-5-methylphenyl)acetyl chloride I147 (1.63 g, 8.2 mmol) dropwise and the mixture was then heated at 165° C. for 1 h. The mixture was allowed to cool to RT, diluted with water and extracted with diethyl ether. The combined organic layers were washed with water, brine, dried over Na₂SO₄, filtered and concentrated under reduced pressure. The residue was purified by silica gel chromatography (Pet. ether/EtOAc=20/1 to 15/1) to give the title compound (2.31 g, 54%) as a brown oil. LCMS-C: Rₜ 1.77 min; m/z 204.0 [M+H]⁺.

### d) 2-(2-(3-Methoxy-5-methylphenyl)-2-(oxazol-2-yl)ethyl)isoindoline-1,3-dione I149

To a solution of 2-(3-methoxy-5-methylbenzyl)oxazole I148 (2.31 g, 11.4 mmol) in anhydrous THF (100 mL) at −78° C. under N₂ was added LiHMDS (1 M solution in THF, 13.7 mL, 13.7 mmol) dropwise. The mixture was stirred at −78° C. for 1 h, then added to a solution of 2-(bromomethyl)isoindoline-1,3-dione (3.29 g, 13.7 mmol) in anhydrous THF (100 mL) dropwise. The mixture was allowed to warm to RT and stirred overnight. The reaction was quenched with a saturated aqueous NH₄Cl solution and the mixture was diluted with water and extracted with DCM (500 mL×3). The combined organic extracts were washed with brine, dried over Na₂SO₄, filtered and concentrated under reduced pressure. The residue was purified by silica gel chromatography (Pet. ether/EtOAc=20/1 to 5/1) to give the title compound (960 mg, 23%) as a yellow oil. LCMS-C: Rₜ 2.28 min; m/z 363.0 [M+H]⁺.

### e) 2-(3-Methoxy-5-methylphenyl)-2-(oxazol-2-yl)ethan-1-amine I150

A mixture of 2-(2-(3-methoxy-5-methylphenyl)-2-(oxazol-2-yl)ethyl)isoindoline-1,3-dione I149 (960 mg, 2.65 mmol) and hydrazine monohydrate (397.5 mg, 7.95 mmol) in EtOH (150 mL) was heated at 80° C. for 3 h. The mixture was then concentrated under reduced pressure and the residue was purified by silica gel chromatography (EtOAc/Pet. ether=50/1 to 2/1) to give the title compound (300 mg, 48%) as a yellow oil. ¹H NMR (400 MHz, DMSO-d₆) δ 8.00 (s, 1H), 7.17 (s, 1H), 6.72-6.42 (m, 3H), 4.18-3.95 (m, 1H), 3.70 (s, 3H), 3.24-3.17 (m, 1H), 3.08-2.86 (m, 1H), 2.23 (s, 3H).

### lii) 2-([1,1′-Biphenyl]-3-yl)-2-(oxazol-2-yl)ethan-1-amine (I151)

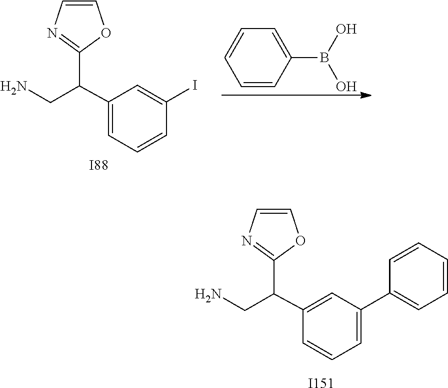

To a solution of 2-(3-iodophenyl)-2-(oxazol-2-yl)ethan-1-amine I88 (100 mg, 0.32 mmol) in DMF (10 mL) and water (2 mL) was added phenylboronic acid (78 mg, 0.64 mmol), Pd(PPh₃)₄ (74 mg, 0.064 mmol) and Cs₂CO₃ (622 mg, 1.9 mmol) and the mixture was heated at 110° C. under N₂ overnight. The mixture was diluted with water, extracted with EtOAc and the organic extract was concentrated under reduced pressure. The residue was purified by prep. TLC (DCM/MeOH=20/1, v/v) to give the title compound (30 mg, 35%) as a yellow solid. LCMS-C: Rₜ 0.55 min, m/z 265.1 [M+H]⁺.

### liii) 3-Amino-2-cyclohexylpropan-1-ol (I155)

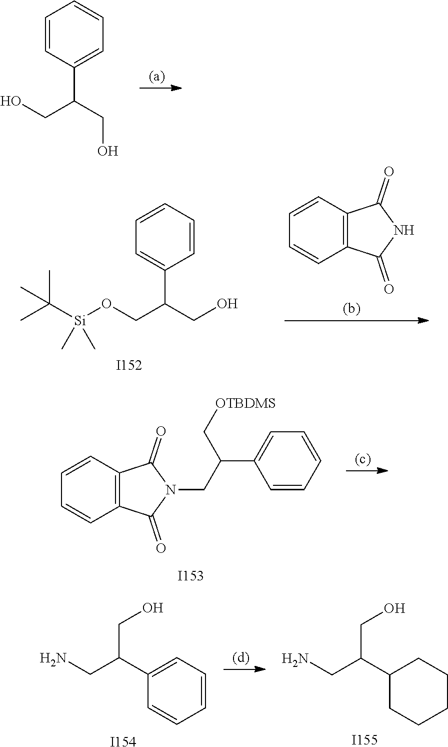

### a) 3-((tert-Butyldimethylsilyl)oxy)-2-phenylpropan-1-ol I152

To a solution of 2-phenylpropane-1,3-diol (5.0 g, 32.9 mmol), TBDMSCI (4.95 g, 32.9 mmol) and DMAP (40 mg, 0.329 mmol) in DCM (60 mL) at 0° C. under N₂ was added Et₃N (3.66 g, 36.2 mmol) and the mixture was stirred at RT for 12 h. The mixture was partitioned between water and DCM, the layers were separated and the organic phase was dried over Na₂SO₄, filtered and concentrated under reduced pressure. The residue was purified by silica gel chromatography (Pet. ether/EtOAc=30/1) to give the title compound (2.75 g, 32%) as a colorless oil. LCMS-C: Rₜ 2.69 min; m/z 267.1 [M+H]⁺.

b) 2-(3-((tert-Butyldimethylsilyl)oxy)-2-phenylpropyl)isoindoline-1,3-dione I153 To an ice-cooled solution of 3-((tert-butyldimethylsilyl)oxy)-2-phenylpropan-1-ol I152 (1.4 g, 5.25 mmol), phthalimide (850 mg, 5.78 mmol) and PPh₃ (1.52 g, 5.78 mmol) in THF (20 mL) was added a solution of DIAD (1.17 g, 5.78 mmol) in THF (10 mL) dropwise and the mixture was stirred at RT overnight. The mixture was partitioned between water and EtOAc, the layers were separated and the organic layer was concentrated under reduced pressure to give the title compound (1.2 g, 58%) as a yellow oil, which was used directly in the next step.

### c) 3-Amino-2-phenylpropan-1-ol I154

A mixture of 2-(3-((tert-butyldimethylsilyl)oxy)-2-phenylpropyl)isoindoline-1,3-dione I153 (1.2 g, 3.03 mmol) and hydrazine monohydrate (445 mg, 9.09 mmol) in EtOH (50 mL) was heated at 80° C. for 3.5 h under N₂. The mixture was allowed to cool to RT, partitioned between water and EtOAc, the layers were separated and the organic layer was concentrated under reduced pressure to give the title compound (660 mg, 83%) as a colorless oil. LCMS-C: Rₜ 0.29 min; m/z 152.0 [M+H]⁺.

### d) 3-Amino-2-cyclohexylpropan-1-ol I155

A mixture of 3-amino-2-phenylpropan-1-ol I154 (100 mg, 0.66 mmol) and Pt₂O (10 mg) in AcOH (5 mL) was stirred at RT under a H₂ atmosphere for 72 h. The catalyst was removed by filtration and the filtrate was concentrated under reduced pressure to give the title compound (87 mg, 84%) as a colorless oil. LCMS (ES-API): Rₜ 0.27 min; m/z 158.1 [M+H]⁺. ¹H NMR (400 MHz, DMSO-d₆) 3.55-3.49 (m, 1H), 3.46-3.39 (m, 1H), 2.78-2.71 (m, 1H), 2.70-2.61 (m, 1H), 1.40-1.28 (m, 2H), 1.20-1.08 (m, 2H), 1.04-0.93 (m, 3H), 0.89-0.83 (m, 5H).

### liv) Ethyl 7-(1H-1,2,3-triazol-4-yl)-2H-benzo[e][1,2,4]thiadiazine-3-carboxylate 1,1-dioxide (I158)

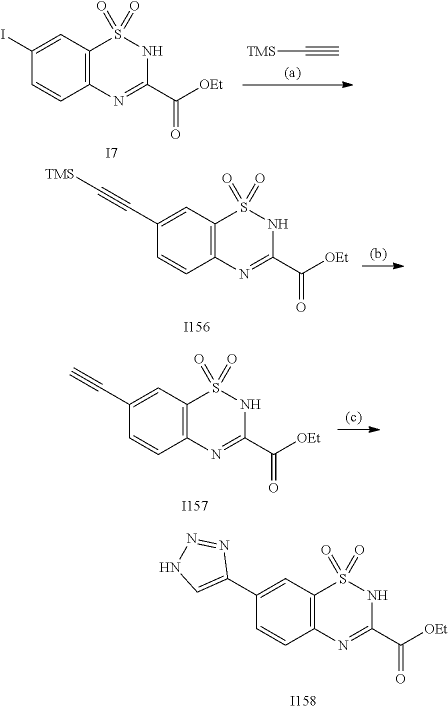

### a) Ethyl 7-((trimethylsilyl)ethynyl)-2H-benzo[e][1,2,4]thiadiazine-3-carboxylate 1,1-dioxide I156

To a mixture of ethyl 7-iodo-2H-benzo[e][1,2,4]thiadiazine-3-carboxylate 1,1-dioxide I7 (1.0 g, 2.63 mmol), CuI (25 mg, 0.13 mmol) and Pd(PPh₃)₂Cl₂(91 mg, 0.13 mmol) in Et₃N (20 mL) and DMF (50 mL) under N₂ was added ethynyltrimethylsilane (1.03 g, 0.1 mmol) and the mixture was stirred at 30° C. overnight. The mixture was partitioned between water and EtOAc, the layers were separated and the organic layer was dried over Na₂SO₄, filtered and concentrated under reduced pressure. The residue was purified by silica gel chromatography (DCM/MeOH=100/1) to give the title compound (350 mg, 38%) as a black solid. LCMS (ES-API): Rₜ 2.43 min; m/z 351.0 [M+H]⁺.

### b) Ethyl 7-ethynyl-2H-benzo[e][1,2,4]thiadiazine-3-carboxylate 1,1-dioxide I157

To a solution of ethyl 7-((trimethylsilyl)ethynyl)-2H-benzo[e][1,2,4]thiadiazine-3-carboxylate 1,1-dioxide I156 (300 mg, 0.86 mmol) in THF (30 mL) was added TBAF (1 M solution in THF, 4.28 mL, 4.28 mmol) and the mixture was heated at 40° C. overnight. The mixture was partitioned between water and EtOAc, the layers were separated and the organic layer was dried over Na₂SO₄, filtered and concentrated under reduced pressure. The residue was purified by silica gel chromatography (DCM/MeOH=100/1) to give the title compound (217 mg, 91%) as an orange solid. LCMS-C: Rₜ 2.58 min; m/z 279.0 [M+H]⁺.

### c) Ethyl 7-(1H-1,2,3-triazol-4-yl)-2H-benzo[e][1,2,4]thiadiazine-3-carboxylate 1,1-dioxide I158

A mixture of ethyl 7-ethynyl-2H-benzo[e][1,2,4]thiadiazine-3-carboxylate 1,1-dioxide I157 (180 mg, 0.65 mmol), azidotrimethylsilane (111.6 mg, 0.97 mmol) and CuI (37 mg, 0.19 mmol) in DMF (7 mL) and EtOH (1 mL) was heated at 120° C. overnight. The mixture was partitioned between water and EtOAc, the layers were separated and the organic layer was dried over Na₂SO₄, filtered and concentrated under reduced pressure. The residue was purified by silica gel chromatography (DCM/MeOH=20/1) to give the title compound (17 mg, 7%) as an orange oil. LCMS-C: Rₜ 0.45 min; m/z 321.9 [M+H]⁺.

### lv) Ethyl 7-(methylsulfonyl)-2H-benzo[e][1,2,4]thiadiazine-3-carboxylate 1,1-dioxide (I161)

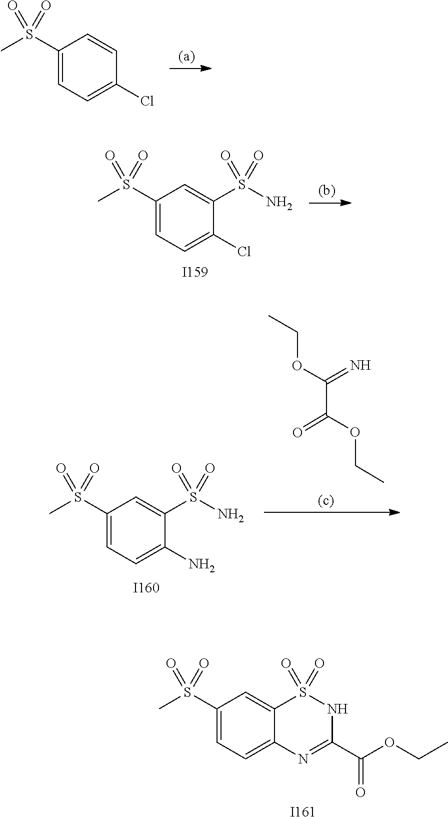

### a) 2-Chloro-5-(methylsulfonyl)benzenesulfonamide I159

1-Chloro-4-(methylsulfonyl)benzene (10.0 g, 5.3 mmol) was slowly added to CISO₃H (63 mL) and the mixture was heated at 100° C. for 1 h. SO₂Cl₂ (3.8 mL) was then added and the mixture was heated at reflux for 2 h, then allowed to cooled to RT and poured into ice-water. The resulting precipitate was collected by filtration and washed with cold water. The solid was dissolved in aqueous NH₄OH solution (10% w/v, 375 mL) and the mixture was stirred at RT for 30 min. The mixture was concentrated under reduced pressure until precipitation occurred and the precipitate was collected by filtration and washed with water. The filter cake was dissolved in an aqueous NaOH solution (10% w/v, 50 mL) and the mixture was adjusted to pH 5 with 6 M aqueous HCl solution. The resulting precipitate was collected by filtration, washed with water and dried to give the title compound (2.0 g, 14%) as a white solid. LCMS-D: Rₜ 1.5 min, m/z 270.0 [M+H]⁺.

### b) 2-Amino-5-(methylsulfonyl)benzenesulfonamide I160

A solution of 2-chloro-5-(methylsulfonyl)benzenesulfonamide I159 (1.0 g, 3.7 mmol) in conc. aqueous NH₄OH (200 mL) was stirred at RT for 4 h. The mixture was concentrated under reduced pressure and the residue was adjusted to pH 5 with 6 M aqueous HCl. The resulting precipitate was collected by filtration, washed with water and dried to give the title compound (500 mg, 54%) as a white solid. LCMS-D: Rₜ 1.70 min, m/z 249.0 [M−H]⁻.

### c) Ethyl 7-(methylsulfonyl)-2H-benzo[e][1,2,4]thiadiazine-3-carboxylate 1,1-dioxide I161

To a solution of 2-amino-5-(methylsulfonyl)benzenesulfonamide I160 (240 mg, 0.96 mmol) and ethyl 2-ethoxy-2-iminoacetate (278 mg, 1.92 mmol) in EtOH (2 mL) was added Et₃N (291 mg, 2.88 mmol) and the mixture was heated at 120° C. under microwave irradiation for 2 h. The solvent was removed under reduced pressure and the residue was purified by prep. TLC (DCM/MeOH=20/1) to give the title compound (50 mg, 16%) as a white solid. LCMS-D: Rₜ 1.70 min, m/z 333.0 [M+H]⁺.

### lvi) Ethyl 7-chloro-2H-benzo[e][1,2,4]thiadiazine-3-carboxylate 1,1-dioxide (I162)

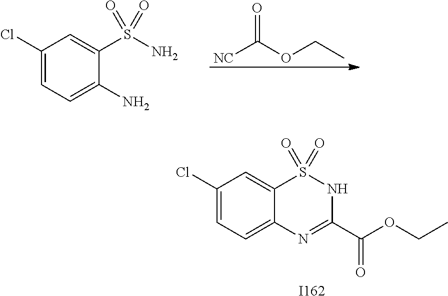

To a solution of 2-amino-5-chlorobenzenesulfonamide (1.0 g, 4.8 mmol) in AcOH (40 mL) was added ethyl carbonocyanidate (4.8 g, 48.0 mmol) and the mixture was stirred at RT under N₂ for 5 min. Concentrated aqueous HCl (1 mL) was then added and the mixture was heated at 85° C. for 4 h. The mixture was concentrated under reduced pressure to remove ˜⅔ of the solvent and then diluted with water (20 mL). The resulting precipitate was collected by filtration and washed with water. The solid was diluted with DCM (60 mL), stirred for 1 h then filtered and the filter cake was rinsed with DCM. The combined filtrates were concentrated under reduced pressure to give the title compound (950 mg, 68%) as a grey solid. LCMS-D: Rₜ 1.05 min; m/z 288.9 [M+H]⁺. ¹H NMR (400 MHz, DMSO-d₆) δ 12.9 (br s, 1H), 7.94 (d, J=2.0 Hz, 1H), 7.84-7.77 (m, 2H), 4.40 (q, J=7.1 Hz, 2H), 1.35 (t, J=7.1 Hz, 3H).

### lviii) 7-Chloro-2H-benzo[e][1,2,4]thiadiazine-3-carboxylic Acid 1,1-dioxide (I163)

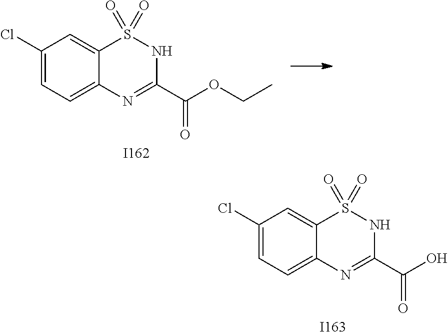 [SMILES: "I162" ; "I163" ; CCOC(=O)C1=Nc2ccc(Cl)cc2S(=O)(=O)N1 ; O=C(O)C1=Nc2ccc(Cl)cc2S(=O)(=O)N1]

To a solution of ethyl 7-chloro-2H-benzo[e][1,2,4]thiadiazine-3-carboxylate 1,1-dioxide I162 (560 mg, 1.94 mmol) in MeOH (75 mL) and water (25 mL) at RT was added NaOH (388 mg, 9.7 mmol) and the mixture was stirred at RT for 4 h. Most of the MeOH was removed under reduced pressure and the aqueous residue was diluted with Et₂O (20 mL). The layers were separated and the organic phase was extracted with water (10 mL). The combined aqueous layers were adjusted to pH 2 with 1 M aqueous HCl and the resulting precipitate was collected by filtration and dried to give the title compound (300 mg, 59%) as a white solid. LCMS-C: Rₜ 0.39 min; m/z 258.9 [M−H]⁻.

### Example 1: 7-Bromo-N-(2-(oxazol-2-yl)-2-phenylethyl)-2H-benzo[e][1,2,4]thiadiazine-3-carboxamide 1,1-dioxide (1)

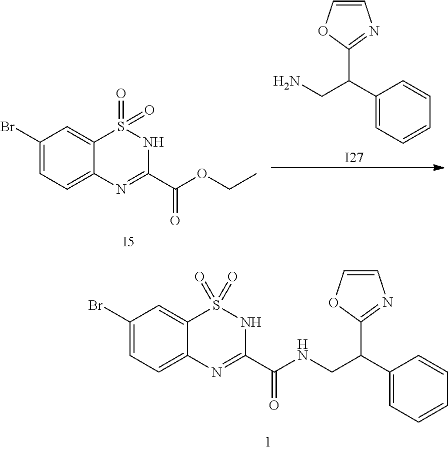

Ethyl 7-bromo-2H-benzo[e][1,2,4]thiadiazine-3-carboxylate 1,1-dioxide (I5) (1.06 g, 3.19 mmol) and 2-(oxazol-2-yl)-2-phenylethanamine (I27) (500 mg, 2.66 mmol) were dissolved in methanol (8 mL) and the mixture was heated in a sealed tube at 130° C. for 3h then cooled to r.t. The mixture was filtered and the filter cake was washed with methanol (5 mL). The combined filtrates were concentrated to give the product (1.00 g, 39% yield) as a white solid. LCMS (ES-API): Rₜ 2.62 min; m/z 475/477 [M+H]⁺. ¹H NMR (400 MHz, d₆-DMSO) δ 12.8 (s, 1H), 9.30 (t, J=5.6 Hz, 1H), 8.05 (s, 1H), 8.01 (d, J=2.0 Hz, 1H), 7.93 (dd, J=8.8 Hz, 2.0 Hz, 1H), 7.76 (d, J=8.4 Hz, 1H), 7.36-7.27 (m, 5H), 7.21 (s, 1H), 4.68 (t, J=7.6 Hz, 1H), 4.05-3.85 (m, 2H).

### Example 2: N-(3-Hydroxy-2-phenylpropyl)-2H-benzo[e][1,2,4]thiadiazine-3-carboxamide 1,1-dioxide (2)

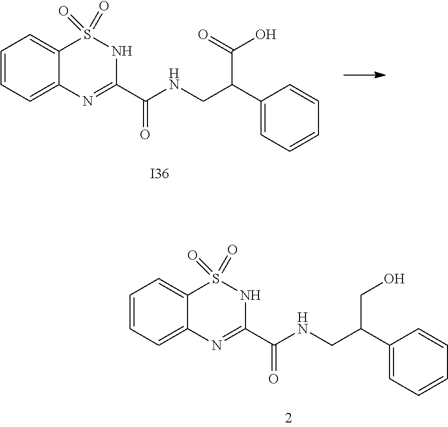 [SMILES: "I36" ; O=C(NCC(C(=O)O)c1ccccc1)C1=Nc2ccccc2S(=O)(=O)N1 ; O=C(NCC(CO)c1ccccc1)C1=Nc2ccccc2S(=O)(=O)N1]

3-(1,1-Dioxido-2H-benzo[e][1,2,4]thiadiazine-3-carboxamido)-2-phenylpropanoic acid (I36) (50 mg, 0.129 mmol) was added into BH₃.THF (2 M in THF, 10 mL) at r.t. under nitrogen and the mixture was stirred at r.t. for 30 min. The solvent was removed under vacuum to give a residue which was purified by preparative TLC (DCM/MeOH=20:1) to give the desired product (25 mg, 54% yield) as a white solid. ¹H NMR (400 MHz, d₆-DMSO) δ 12.6 (s, 1H), 9.13 (t, J=6.0 Hz, 1H), 7.85-7.79 (m, 2H), 7.74-7.72 (m, 1H), 7.54 (t, J=8.0 Hz, 1H), 7.31-7.21 (m, 5H), 4.84 (t, J=4.8 Hz, 1H), 3.60-3.58 (m, 4H), 3.17-3.10 (m, 1H); LCMS (ES-API): Rₜ 2.10 min, m/z 360.1 [M+H]⁺

### Example 3: N-(4-Hydroxy-2-phenylbutyl)-2H-benzo[e][1,2,4]thiadiazine-3-carboxamide 1,1-dioxide (3)

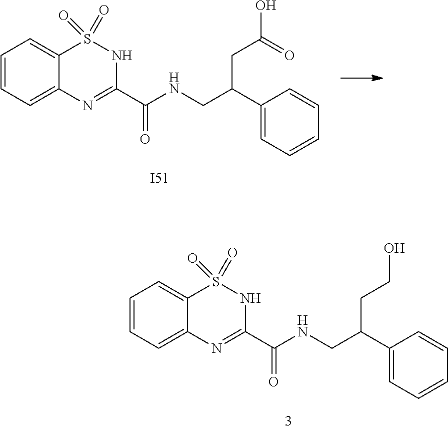 [SMILES: "I51" ; O=C(O)CC(CNC(=O)C1=Nc2ccccc2S(=O)(=O)N1)c1ccccc1 ; O=C(NCC(CCO)c1ccccc1)C1=Nc2ccccc2S(=O)(=O)N1]

4-(1,1-Dioxido-2H-benzo[e][1,2,4]thiadiazine-3-carboxamido)-3-phenylbutanoic acid (I51) (80 mg, 0.206 mmol) was added into BH₃.THF (2 M in THF, 40 mL) at r.t. under nitrogen and the mixture was stirred at r.t. for 3 h. The solvent was removed under vacuum to give a residue which was purified by preparative TLC (DCM/MeOH=20:1) to give the desired product (40 mg, 52% yield) as a white solid. ¹H NMR (400 MHz, d₆-DMSO) δ 12.6 (s, 1H), 9.17 (t, J=6.0 Hz, 1H), 7.85 (d, J=8.0 Hz, 1H), 7.80 (d, J=8.0 Hz, 1H), 7.74-7.70 (m, 1H), 7.54 (t, J=8.0 Hz, 1H), 7.31-7.28 (m, 2H), 7.23-7.18 (m, 3H), 4.49 (t, J=4.8 Hz, 1H), 3.03-3.05 (m, 5H), 1.93-1.86 (m, 1H), 1.73-1.62 (m, 1H); LCMS (ES-API): Rₜ 2.18 min, m/z 374.1 [M+H]⁺

### Example 4: 7-Isocyano-N-(2-(oxazol-2-yl)-2-phenylethyl)-2H-benzo[e][1,2,4]thiadiazine-3-carboxamide 1,1-dioxide (4)

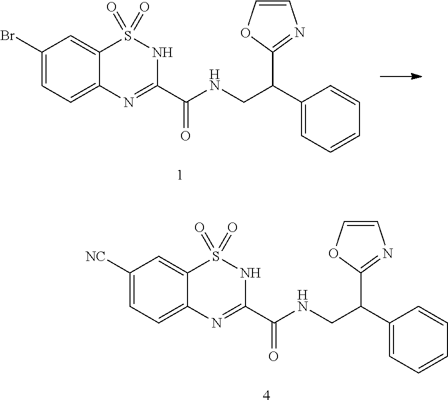

A mixture of 7-bromo-N-(2-(oxazol-2-yl)-2-phenylethyl)-2H-benzo[e][1,2,4]thiadiazine-3-carboxamide 1,1-dioxide (1) (50 mg, 0.105 mmol), Zn(CN)₂ (62 mg, 0.525 mmol), Pd₂(dba)₃ (19 mg, 0.021 mmol), Xantphos (18 mg, 0.0315 mmol) and Cs₂CO₃ (171 mg, 0.525 mmol) in DMF (3 mL) was heated at 160° C. in a microwave reactor for 30 min. The mixture was partitioned between dichloromethane and water and the aqueous layer was adjusted to pH 2-3 with aqueous HCl. The layers were separated and the aqueous phase was washed with water, brine and dried over Na₂SO₄. The solvent was removed under vacuum and the residue was purified by preparative TLC (DCM/MeOH=50:1) to give the desired product (25 mg, 57% yield) as a white solid. ¹H NMR (400 MHz, d₆-DMSO) δ 12.9 (s, 1H), 9.35 (t, J=6.4 Hz, 1H), 8.48 (d, J=1.6 Hz, 1H), 8.15 (dd, J=8.4, 1.6 Hz, 1H), 8.05 (s, 1H), 7.92 (d, J=8.8 Hz, 1H), 7.36-7.27 (m, 5H), 7.21 (s, 1H), 4.69 (t, J=7.6 Hz, 1H), 4.05-3.98 (m, 1H), 3.91-3.85 (m, 1H); LCMS (ES-API): Rₜ 2.10 min, m/z 422.1 [M+H]⁺

### Example 5: N-(2-(Oxazol-2-yl)-2-phenylethyl)-7-(trifluoromethoxy)-2H-benzo[e][1,2,4]thiadiazine-3-carboxamide 1,1-dioxide (5)

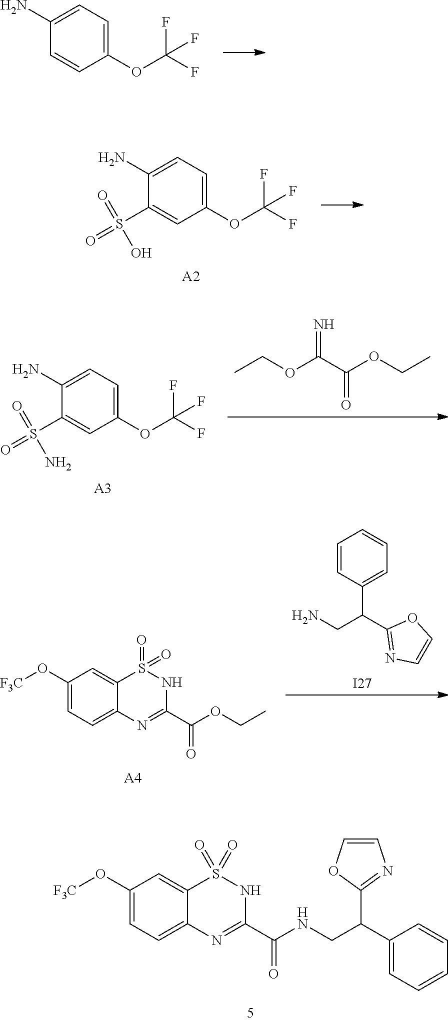

### a) 2-Amino-5-(trifluoromethoxy)benzenesulfonic acid (A2)

To a solution of 4-(trifluoromethoxy)aniline (20 g, 0.113 mol) in 1, 2, 4-trichlorobenzene (100 mL) at 100° C. was added H₂₅₀₄ dropwise (95%, 15.2 g). After addition, the mixture was heated at 210° C. for 3 h, cooled to r.t. and then basified with Na₂CO₃ (sat. aq.). The mixture was then washed with DCM and the aqueous layer was acidified to pH 2 with 1 M HCl. The resulting precipitate was collected by filtration and dried to give the product (10 g, 34% yield) as an off-white solid. LCMS (ES-API): Rₜ 1.25 min; m/z 256.0 [M−H]⁻.

### b) 2-Amino-5-(trifluoromethoxy)benzenesulfonamide (A3)

To a solution of 2-amino-5-(trifluoromethoxy)benzenesulfonic acid (A2) (3.5 g, 13.61 mmol) in tetrahydrothiophene 1,1-dioxide (15 mL) at r.t. was added POCl₃ (6.26 g, 40.82 mmol) and the mixture was heated at 120° C. for 3 h. After cooling, the mixture was added dropwise to a solution of conc. NH₄OH (100 mL) at 0° C. and stirred for 30 min. The mixture was extracted with EtOAc, the organic layer was dried (Na₂SO₄), filtered, concentrated and purified by column chromatography (EtOAc/Pet. Ether=1:1) to give the product (1.4 g, crude) which was used directly in the next step. LCMS (ES-API): Rₜ 2.06 min; m/z 257.0 [M+H]⁺.

### c) Ethyl 7-(trifluoromethoxy)-2H-benzo[e][1,2,4]thiadiazine-3-carboxylate 1,1-dioxide (A4)

A mixture of 2-amino-5-(trifluoromethoxy)benzenesulfonamide (A3) (800 mg, 3.12 mmol), ethyl 2-ethoxy-2-iminoacetate (680 mg, 4.68 mmol) and TEA (631 mg, 6.24 mmol) in EtOH (20 mL) was heated at 85° C. for 8 h. The mixture was then poured into water and extracted with EtOAc. The organic layer was washed with 1 M HCl, dried (Na₂SO₄), filtered, concentrated and purified by column chromatography (EtOAc/Pet. Ether=1:1) to give the product (200 mg, 19% yield) as a yellow solid. LCMS (ES-API): Rₜ 2.41 min; m/z 339.0 [M+H]⁺.

### d) N-(2-(Oxazol-2-yl)-2-phenylethyl)-7-(trifluoromethoxy)-2H-benzo[e][1,2,4]thiadiazine-3-carboxamide 1,1-dioxide (5)

A mixture of ethyl 7-(trifluoromethoxy)-2H-benzo[e][1,2,4]thiadiazine-3-carboxylate 1,1-dioxide (A4) (80 mg, 0.24 mmol) and 2-(oxazol-2-yl)-2-phenylethanamine (I27) (45 mg, 0.24 mmol) in EtOH (2 mL) was heated at 130° C. for 2 h. After cooling, the mixture was purified directly by preparative TLC (DCM/MeOH=20:1) to give the product (75 mg, 66% yield) as a white solid. LCMS (ES-API): Rₜ 2.71 min; m/z 481.0 [M+H]⁺. ¹H NMR (400 MHz, d₆-DMSO) δ 12.9 (s, 1H), 9.32 (t, J=5.6 Hz, 1H), 8.05 (s, 1H), 7.95 (d, J=9.2 Hz, 1H), 7.86 (s, 1H), 7.81 (d, J=8.8 Hz, 1H), 7.36-7.26 (m, 5H), 7.21 (s, 1H), 4.68 (t, J=7.6 Hz, 1H), 4.05-3.99 (m, 1H), 3.92-3.86 (m, 1H).

### Example 6: N-(2-(2-Methylpyridin-3-yl)phenethyl)-2H-benzo[e][1,2,4]thiadiazine-3-carboxamide 1,1-dioxide (6)

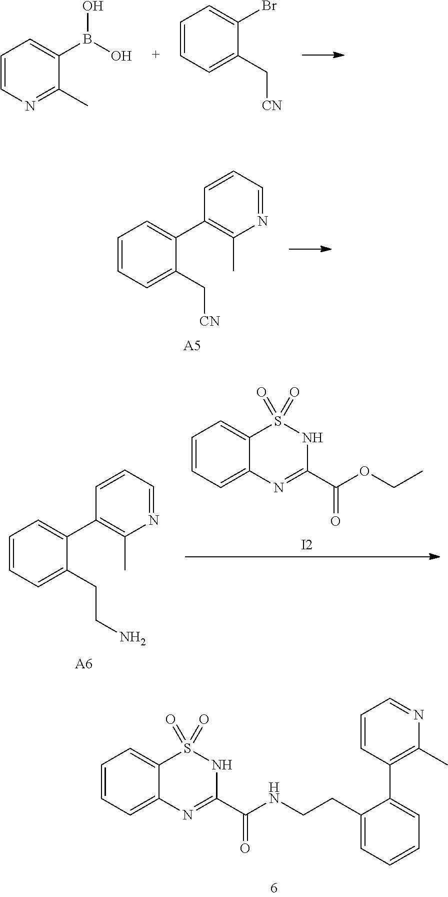

### a) 2-(2-(2-Methylpyridin-3-yl)phenyl)acetonitrile (A5)

(2-Methylpyridin-3-yl)boronic acid (550 mg, 3.2 mmol), 2-(2-bromophenyl)acetonitrile (597 mg, 3.05 mmol), Pd(PPh₃)₄ (176 mg, 0.15 mmol) and K₂CO₃ (176 mg, 0.15 mmol) were dissolved in iPrOH (5 mL) and water (2 mL) and the mixture was heated at 80° C. under N₂ for 5h. The mixture was filtered and the solid was washed with DCM (20 mL). The filtrate was washed with brine, dried over sodium sulfate and concentrated. Column chromatography (DCM/MeOH=100:0-20:1) gave the product (300 mg, 45% yield) as a yellow solid. LCMS (ES-API): Rₜ 0.44 min; m/z 209.1 [M+H]⁺.

### b) 2-(2-(2-Methylpyridin-3-yl)phenyl)ethanamine (A6)

A mixture of 2-(2-(2-methylpyridin-3-yl)phenyl)acetonitrile (A5) (300 mg, 1.4 mmol), NaOH (I173 mg, 4.3 mmol) and Raney-Ni (100 mg) in THF (5 mL) and water (2 mL) was heated at 60° C. under H₂ for 5 h. The mixture was filtered and the solid was washed with DCM (20 mL). The filtrate was washed with brine, dried over sodium sulfate and concentrated to give the product (200 mg, 65% yield) as a white solid. LCMS (ES-API): Rₜ 0.29 min; m/z 213.1 [M+H]⁺.

### c) N-(2-(2-Methylpyridin-3-yl)phenethyl)-2H-benzo[e][1,2,4]thiadiazine-3-carboxamide 1,1-dioxide (6)

A mixture of 2-(2-(2-Methylpyridin-3-yl)phenyl)ethanamine (A6) (35 mg, 0.17 mmol), ethyl 2H-benzo[e][1,2,4]thiadiazine-3-carboxylate 1,1-dioxide (12) (50 mg, 0.20 mmol) and triethylamine (0.2 mL) in methanol (3 mL) was heated in a sealed tube at 130° C. for 3h. The mixture was allowed to cool to r.t., adjusted to pH 5 with 1 M HCl and extracted with DCM (10 mL×3). The combined organic extracts were washed with brine, dried over sodium sulfate and concentrated to give a residue which was purified by preparative TLC (MeOH/DCM=1:20) to give the product (5 mg, 10% yield) as an off-white solid. LCMS (ES-API): Rₜ 1.63 min; m/z 421.1 [M+H]⁺. ¹H NMR (400 MHz, d₆-DMSO) δ 12.5 (s, 1H), 9.20 (t, J=5.6 Hz, 1H), 8.46 (dd, J=4.8 Hz, 1.6 Hz, 1H), 7.85-7.79 (m, 2H), 7.74-7.70 (m, 1H), 7.54-7.50 (m, 2H), 7.41-7.29 (m, 3H), 7.24-7.21 (m, 1H), 7.11 (d, J=7.2 Hz, 1H), 3.32-3.27 (m, 2H), 2.27-2.65 (m, 1H), 2.58-2.52 (m, 1H), 2.20 (s, 3H).

### Example 7: N-(2-(Oxazol-2-yl)phenethyl)-2H-benzo[e][1,2,4]thiadiazine-3-carboxamide1,1-dioxide (7)

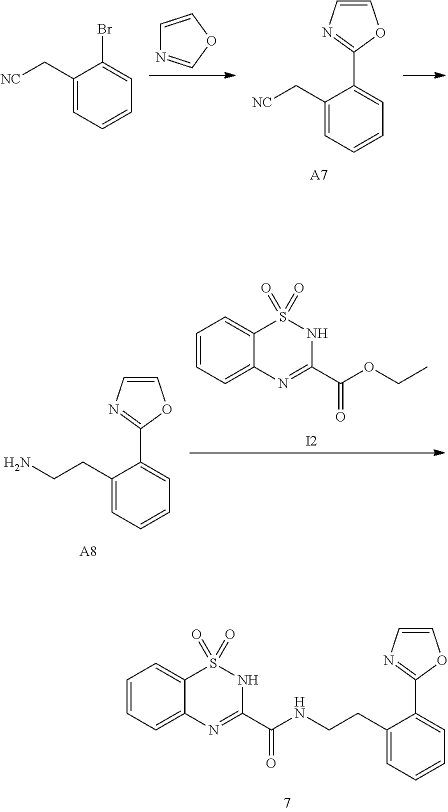

### a) 2-(2-(Oxazol-2-yl)phenyl)acetonitrile (A7)

To a solution of oxazole (1.0 g, 10.2 mmol) in THF (30 mL) at −78° C. was added n-BuLi (2.5 M in hexanes, 6.8 mL, 17.0 mmol) dropwise and the mixture was stirred at −78° C. for 10 min. ZnCl₂ (4.17 g, 30.6 mmol) was added and the mixture was allowed to warm to r.t. Pd(PPh₃)₄ (577 mg, 0.5 mmol) and 2-(2-bromophenyl)acetonitrile (2.0 g, 14.3 mmol) were added and the mixture was heated at 60° C. overnight. The reaction was quenched by addition of saturated aqueous ammonium chloride solution (40 mL) and then most of the THF was removed under reduced pressure. The aqueous mixture was extracted with EtOAc (50 mL×3) and the combined extracts were dried over anhydrous sodium sulfate and concentrated. The residue was purified by column chromatography (EtOAc/Pet. ether=1:10) to afford the desired product (120 mg, 7% yield) as yellow oil. LCMS (ES-API): Rₜ 2.20 min; m/z 185.1 [M+H]⁺.

### b) 2-(2-(Oxazol-2-yl)phenyl)ethanamine (A8)

To a solution of 2-(2-(oxazol-2-yl)phenyl)acetonitrile (A7) (120 mg, 0.65 mmol) in ethanol (3 mL) was added conc. NH₄OH (1 mL) and Raney Nickel (40 mg, 0.68 mmol) and the mixture was heated at 60° C. under a hydrogen (1 atm) overnight. More ethanol (5 mL) was added and the mixture was filtered. The filtrate was concentrated to afford the desired product (110 mg, 80% yield) as a white solid. LCMS (ES-API): Rₜ 2.25 min; m/z 189.1 [M+H]⁺.

### c) N-(2-(Oxazol-2-yl)phenethyl)-2H-benzo[e][1,2,4]thiadiazine-3-carboxamide1,1-dioxide (7)

To a solution of 2-(2-(oxazol-2-yl)phenyl)ethanamine (A8) (110 mg, 0.58 mmol) in ethanol (3 mL) was added ethyl 2H-benzo[e][1,2,4]thiadiazine-3-carboxylate 1,1-dioxide (12) (150 mg, 0.58 mmol) and the mixture was heated at 120° C. for 2 h. The mixture was adjusted to ˜pH 3 with 1 M HCl, diluted with water (10 mL) and extracted with EtOAc (10 mL×3). The combined organic layers were washed with brine, dried over anhydrous sodium sulfate, concentrated and the residue was purified by preparative TLC (DCM/EtOAc=15:1) to give the desired product (40 mg, 18% yield) as a white solid. ¹H NMR (400 MHz, d₆-DMSO) δ 12.6 (s, 1H), 9.38 (t, J=5.8 Hz, 1H), 8.22 (s, 1H), 7.91-7.89 (m, 1H), 7.86-7.84 (m, 1H), 7.81-7.79 (m, 1H), 7.75-7.70 (m, 1H), 7.55-7.50 (m, 1H), 7.46-7.37 (m, 4H), 3.61-3.56 (m, 2H), 3.38-3.37 (m, 2H). LCMS (ES-API): Rₜ 2.49 min; m/z 397.0 [M+H]⁺

### Example 8: 7-Hydroxy-N-(2-(oxazol-2-yl)-2-phenylethyl)-2H-benzo[e][1,2,4]thiadiazine-3-carboxamide 1,1-dioxide (8)

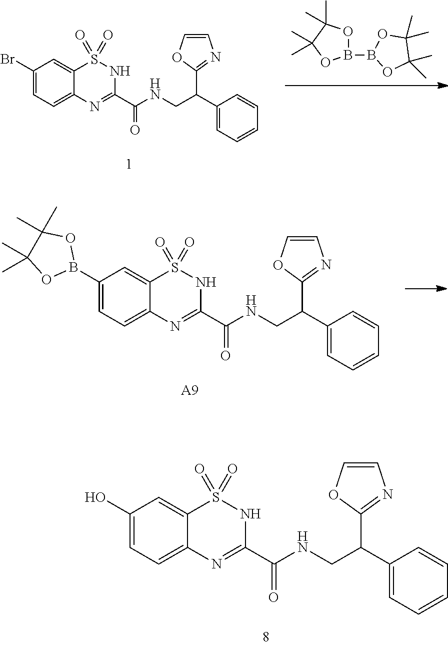

### a) (N-(2-(Oxazol-2-yl)-2-phenylethyl)-7-(4,4,5,5-tetramethyl-1,3,2-dioxaborolan-2-yl)-2H-benzo[e][1,2,4]thiadiazine-3-carboxamide 1,1-dioxide (A9)

To a solution of 7-bromo-N-(2-(oxazol-2-yl)-2-phenylethyl)-2H-benzo[e][1,2,4]thiadiazine-3-carboxamide 1,1-dioxide (1) (400 mg, 0.84 mmol) and bis(pinacolato)diboron (427 mg, 1.68 mmol) in dioxane (20 mL) was added Pd(dppf)₂Cl₂ (69 mg, 0.084 mmol) and KOAc (248 mg, 2.52 mmol) and the mixture was heated at 90° C. under N₂ for 3 h. After cooling to r.t., the mixture was adjusted to pH 5 with 1 M HCl and filtered. The filter cake was washed with dioxane (5 mL) and the filtrate was washed with brine, dried over sodium sulfate and concentrated. The residue which was purified by preparative TLC (MeOH/DCM=1:20) to give the product (80 mg, 18% yield) as a white solid. LCMS (ES-API): Rₜ 2.36 min; m/z 441 [M+H]⁺ (boronic acid).

### b) 7-Hydroxy-N-(2-(oxazol-2-yl)-2-phenylethyl)-2H-benzo[e][1,2,4]thiadiazine-3-carboxamide 1,1-dioxide (8)

To a solution of N-(2-(oxazol-2-yl)-2-phenylethyl)-7-(4,4,5,5-tetramethyl-1,3,2-dioxaborolan-2-yl)-2H-benzo[e][1,2,4]thiadiazine-3-carboxamide 1,1-dioxide (A9) (82 mg, 0.16 mmol) in THF (3 mL) and water (0.5 mL) was added NaOH (19 mg, 0.48 mmol) and H₂O₂ (27 mg, 0.79 mmol) and the mixture was stirred at r.t. for 3 h. The mixture was extracted with DCM (3×10 mL) and the combined organic extracts were washed with brine, dried over sodium sulfate and concentrated to give a residue which was purified by prep. TLC (MeOH/DCM=1:20) to give the product (20 mg, 30% yield) as an off-white solid. LCMS (ES-API): Rₜ 2.28 min; m/z 413.1 [M+H]⁺. ¹H NMR (400 MHz, MeOD) δ 7.85 (s, 1H), 7.46 (d, J=8.8 Hz, 1H), 7.34-7.24 (m, 5H), 7.18-7.11 (m, 3H), 4.61 (t, J=8.0 Hz, 1H), 4.09-3.93 (m, 2H).

### Example 9: 7-(1-(2-(Methylamino)-2-oxoethyl)-1H-pyrazol-4-yl)-N-(2-(oxazol-2-yl)-2-phenylethyl)-2H-benzo[e][1,2,4]thiadiazine-3-carboxamide 1,1-dioxide (9)

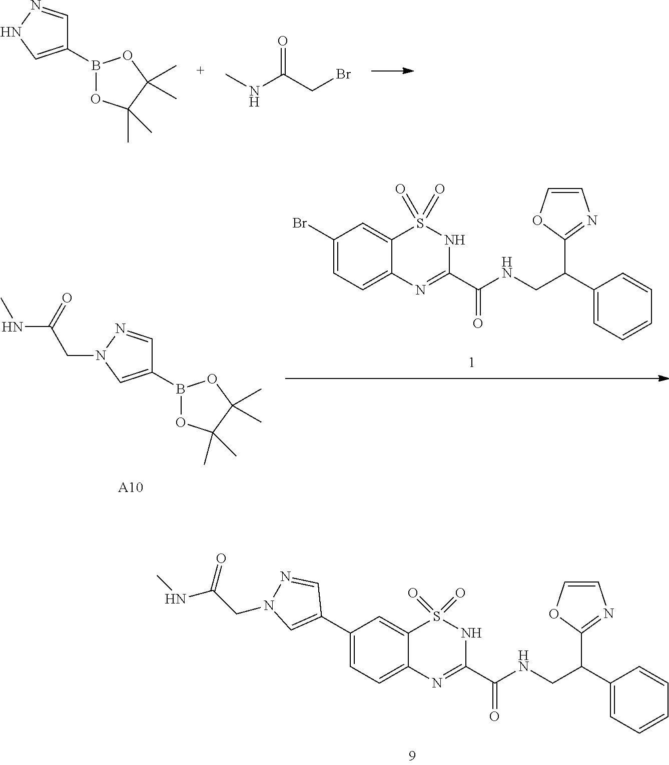

### a) N-Methyl-2-(4-(4,4,5,5-tetramethyl-1,3,2-dioxaborolan-2-yl)-1H-pyrazol-1-yl)acetamide (A10)

To a solution of 4-(4,4,5,5-tetramethyl-1,3,2-dioxaborolan-2-yl)-1H-pyrazole (200 mg, 1.03 mmol) in DMF (10 mL) was added 2-bromo-N-methylacetamide (172 mg, 1.13 mmol) and cesium carbonate (670 mg, 2.06 mmol) and the mixture was heated at 60° C. overnight. The mixture was filtered and the solid was washed with EtOAc. The filtrates were combined and the solvent was removed to give the desired product (160 mg, 59% yield) as a white solid. LCMS (ES-API): Rₜ 1.89 min; m/z 266.1 [M+H]⁺.

### b) 7-(1-(2-(Methylamino)-2-oxoethyl)-1H-pyrazol-4-yl)-N-(2-(oxazol-2-yl)-2-phenylethyl)-2H-benzo[e][1,2,4]thiadiazine-3-carboxamide 1,1-dioxide (9)

To a solution of N-methyl-2-(4-(4,4,5,5-tetramethyl-1,3,2-dioxaborolan-2-yl)-1H-pyrazol-1-yl)acetamide (A10) (70 mg, 0.25 mmol) and 7-bromo-N-(2-(oxazol-2-yl)-2-phenylethyl)-2H-benzo[e][1,2,4]thiadiazine-3-carboxamide 1,1-dioxide (1) (100 mg, 0.21 mmol) in i-PrOH (3 mL) and toluene (1 mL) was added sodium carbonate (2 M in water, 0.32 mL, 0.63 mmol) and Pd(PPh₃)₄ (12 mg, 0.01 mmol) and the mixture was heated at 90° C. under a nitrogen atmosphere overnight. The solvent was removed and the residue was diluted with water and extracted with EtOAc. The organic extract was dried over sodium sulfate, concentrated and the residue was purified by preparative TLC (DCM/MeOH=15:1) to give the desired product (100 mg, 89% yield) as a yellow solid. ¹H NMR (400 MHz, d₆-DMSO) δ 12.6 (s, 1H), 9.22-9.19 (m, 1H), 8.33 (s, 1H), 8.06-7.93 (m, 5H), 7.78-7.76 (m, 1H), 7.35-7.25 (m, 5H), 7.20 (s, 1H), 4.79 (s, 2H), 4.67 (m, 1H), 4.05-3.97 (m, 1H), 3.92-3.85 (m, 1H), 2.63 (d, J=4.5 Hz, 3H). LCMS (ES-API): Rₜ 2.37 min, m/z 534.2 [M+H]⁺

### Example 10: 7-(1-(2-Hydroxyethyl)-1H-pyrazol-4-yl)-N-(2-(oxazol-2-yl)-2-phenylethyl)-2H-benzo[e][1,2,4]thiadiazine-3-carboxamide 1,1-dioxide (10)

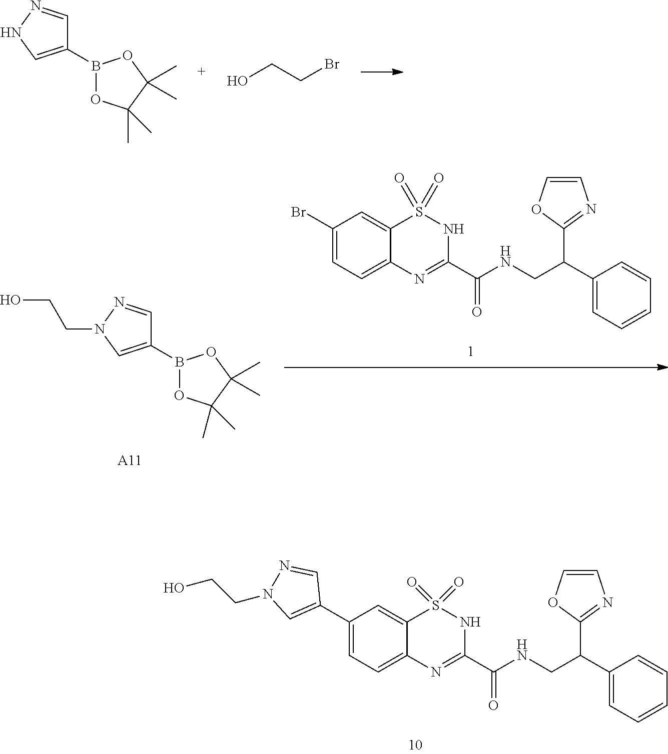

### a) 2-(4-(4,4,5,5-Tetramethyl-1,3,2-dioxaborolan-2-yl)-1H-pyrazol-1-yl)ethanol (A11)

To a solution of 4-(4,4,5,5-tetramethyl-1,3,2-dioxaborolan-2-yl)-1H-pyrazole (500 mg, 2.58 mmol) in DMF (7 mL) was added 2-bromoethanol (645 mg, 5.16 mmol) and cesium carbonate (2.52 g, 7.74 mmol) and the mixture was heated at 85° C. for 3 h. More cesium carbonate (2.52 g, 7.74 mmol) and 2-bromoethanol (645 mg, 5.16 mmol) were added and the mixture was again heated at 85° C. overnight. The solvent was removed and the residue was diluted with water and extracted with EtOAc. The organic layer was washed with water and brine, dried over anhydrous sodium sulfate, filtered and the solvent was evaporated to give the desired product (150 mg, 24% yield) as a yellow oil. LCMS (ES-API): Rₜ 2.0 min; m/z 239.1 [M+H]⁺.

### b) 7-(1-(2-Hydroxyethyl)-1H-pyrazol-4-yl)-N-(2-(oxazol-2-yl)-2-phenylethyl)-2H-benzo[e][1,2,4]thiadiazine-3-carboxamide 1,1-dioxide (10)

To a solution of 2-(4-(4,4,5,5-tetramethyl-1,3,2-dioxaborolan-2-yl)-1H-pyrazol-1-yl)ethanol (A11) (70 mg, 0.29 mmol) and 7-bromo-N-(2-(oxazol-2-yl)-2-phenylethyl)-2H-benzo[e][1,2,4]thiadiazine-3-carboxamide 1,1-dioxide (1) (93 mg, 0.2 mmol) in dioxane (3 mL) was added K₂CO₃ (82 mg, 0.59 mmol) and Pd(dppf)Cl₂ (17 mg, 0.02 mmol) and the mixture heated at 130° C. in a sealed tube for 5 h. Water (20 mL) was added and the mixture was extracted with EtOAc (20 mL×2). The combined organic extracts were dried over sodium sulfate, filtered and concentrated and the residue was purified by prep. TLC (DCM/MeOH=15:1) to give the desired product (10 mg, 10% yield) as a white solid. ¹H NMR (400 MHz, d₆-DMSO) δ 12.6 (s, 1H), 9.19 (s, 1H), 8.33 (s, 1H), 8.03 (m, 2H), 7.99-7.90 (m, 2H), 7.74-7.72 (m, 1H), 7.36-7.32 (m, 2H), 7.29-7.27 (m, 3H), 7.21 (s, 1H), 4.93 (t, J=5.2 Hz, 1H), 4.67 (m, 1H), 4.15 (t, J=5.4 Hz, 2H), 4.05-3.97 (m, 1H), 3.94-3.86 (m, 1H), 3.79-3.75 (m, 2H). LCMS (ES-API): Rₜ 2.48 min, m/z 507.1 [M+H]+

### Example 11: 7-Amino-N-(2-(oxazol-2-yl)-2-phenylethyl)-2H-benzo[e][1,2,4]thiadiazine-3-carboxamide 1,1-dioxide (11)

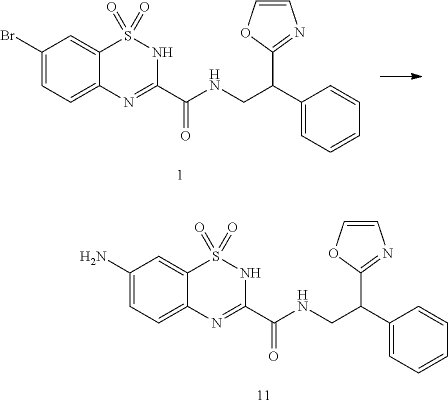

To a solution of 7-bromo-N-(2-(oxazol-2-yl)-2-phenylethyl)-2H-benzo[e][1,2,4]thiadiazine-3-carboxamide 1,1-dioxide (1) (80 mg, 0.17 mmol) and diphenylmethanimine (91.5 mg, 0.51 mmol) in dioxane (5 mL) was added Pd₂(dba)₃ (15.4 mg, 0.02 mmol), Xantphos (19.5 mg, 0.03 mmol) and Cs₂CO₃ (164.5 mg, 0.5 mmol) and the mixture was heated at 90° C. under N₂ for 3 h. The mixture was filtered and the solid was washed with dioxane (5 mL). The filtrate was washed with brine, dried over sodium sulfate and concentrated. The residue was dissolved in dioxane (2 mL) and 1 M HCl (2 mL) was added. The mixture was stirred at r.t. for 1 h then extracted with DCM (3×10 mL). The combined organic extracts were washed with brine, dried over sodium sulfate and concentrated. The residue was purified by preparative TLC (MeOH/DCM=1:20) to give the product (10 mg, 10% yield) as a white solid. LCMS (ES-API): Rₜ 2.17 min; m/z 412.1 [M+H]⁺. ¹H NMR (400 MHz, MeOD) δ 7.87 (s, 1H), 7.26-7.30 (m, 6H), 7.19 (s, 1H), 7.06 (d, J=2.4 Hz, 1H), 7.02-6.99 (m, 1H), 4.63 (t, J=7.2 Hz, 1H), 4.20-3.80 (m, 2H).

### Example 12: Methyl 2-(2-(7-iodo-1,1-dioxido-2H-benzo[e][1,2,4]thiadiazine-3-carboxamido)ethyl)benzoate (12)

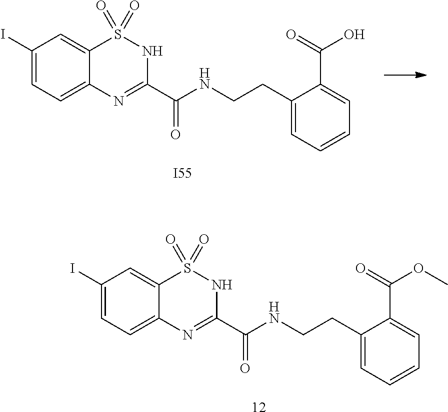

To a solution of 2-(2-(7-iodo-1,1-dioxido-2H-benzo[e][1,2,4]thiadiazine-3-carboxamido)ethyl)benzoic acid (155) (20 mg, 0.06 mmol) in MeOH (5 mL) was added H₂SO₄ (1 drop) and the mixture was heated at 60° C. for 3 h. After cooling to r.t., the mixture was diluted with water (5 mL) and extracted with EtOAc (8 mL×3). The combined organic extracts were dried over Na₂SO₄ and concentrated to give the product (20 mg, 40% yield) as a white solid. ¹H NMR (400 MHz, d₆-DMSO) δ 12.7 (brs, 1H), 9.22 (t, J=5.3 Hz, 1H), 8.09-8.02 (m, 2H), 7.78 (dd, J=8.0, 1.2 Hz, 1H), 7.57 (d, J=8.7 Hz, 1H), 7.54-7.48 (m, 1H), 7.37-7.31 (m, 2H), 3.84 (s, 3H), 3.55-3.49 (m, 2H), 3.16 (t, J=7.0 Hz, 2H). LCMS (ES-API): Rₜ 2.84 min, m/z 513.7 [M+H]⁺

### Example 13: 7-Iodo-N-(2-(oxazol-2-yl)phenethyl)-2H-benzo[e][1,2,4]thiadiazine-3-carboxamide 1,1-dioxide (13)

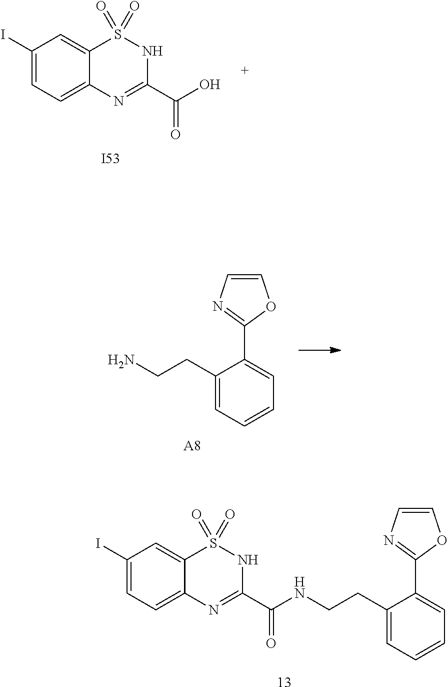

To a solution of 7-iodo-2H-benzo[e][1,2,4]thiadiazine-3-carboxylic acid 1,1-dioxide (I53) (26 mg, 0.14 mmol) and 2-amino-5-bromobenzenesulfonamide (A8) (50 mg, 0.14 mmol) in DCM (10 mL) was added EDCI (55 mg. 0.28 mmol), HOBt (2 mg, 0.01 mmol) and DIPEA (72 mg, 0.56 mmol) and the mixture was stirred at r.t. overnight. A saturated aqueous NaHCO₃ solution (30 mL) was added and the mixture was extracted with DCM (30 mL×3). The combined organic extracts were washed with brine, dried over Na₂SO₄ and concentrated. The residue was purified by prep. TLC (DCM/MeOH=20:1) to give the product (3 mg, 4% yield) as a yellow solid. ¹H NMR (400 MHz, d₆-DMSO) δ 12.7 (s, 1H), 9.36 (brs, 1H), 8.22 (s, 1H), 8.08 (s, 1H), 8.05 (d, J=8.8 Hz, 1H), 7.90 (d, J=8.8 Hz, 1H), 7.58 (d, J=8.4 Hz, 1H), 7.46-7.38 (m, 4H), 3.60-3.55 (m, 2H), 3.51-3.48 (m, 2H). LCMS (ES-API): Rₜ 2.8 min m/z 523.0 [M+H]⁺.

### Example 14: 7-Iodo-N-(2-(methoxymethyl)phenethyl)-2H-benzo[e][1,2,4]thiadiazine-3-carboxamide 1,1-dioxide (14)

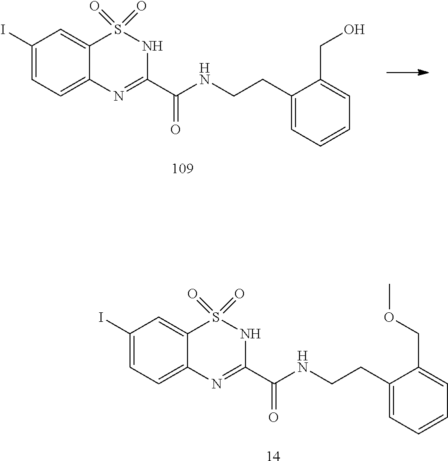

To a solution of N-(2-(hydroxymethyl)phenethyl)-7-iodo-2H-benzo[e][1,2,4]thiadiazine-3-carboxamide 1,1-dioxide (109) (60 mg, 0.12 mmol) in ACN (5 mL) was added Ag₂O (150 mg, 0.6 mmol) and CH₃₁ (180 mg, 1.2 mmol) and the mixture was heated at 50° C. under N₂ overnight. The solids were removed by filtration and the filtrate was concentrated under reduced pressure. The residue was purified by prep. TLC (CH₂Cl₂/MeOH=20:1) to give the product (10 mg, 16% yield) as a white solid. ¹H NMR (400 MHz, d₆-DMSO) δ 12.7 (brs, 1H), 9.33 (m, 1H), 8.10-8.05 (m, 2H), 7.61 (d, J=8.7 Hz, 1H), 7.32 (d, J=7.3 Hz, 1H), 7.26-7.20 (m, 3H), 4.48 (s, 2H), 3.47-3.44 (m, 2H), 3.32 (s, 3H), 2.88 (t, J=7.6 Hz, 2H). LCMS (ES-API): Rₜ 2.75 min; m/z 522.0 [M+H]⁺.

### Example 15: 7-Bromo-N-(2,2-diphenylethyl)-2H-benzo[e][1,2,4]thiadiazine-3-carboxamide 1,1-dioxide (15)

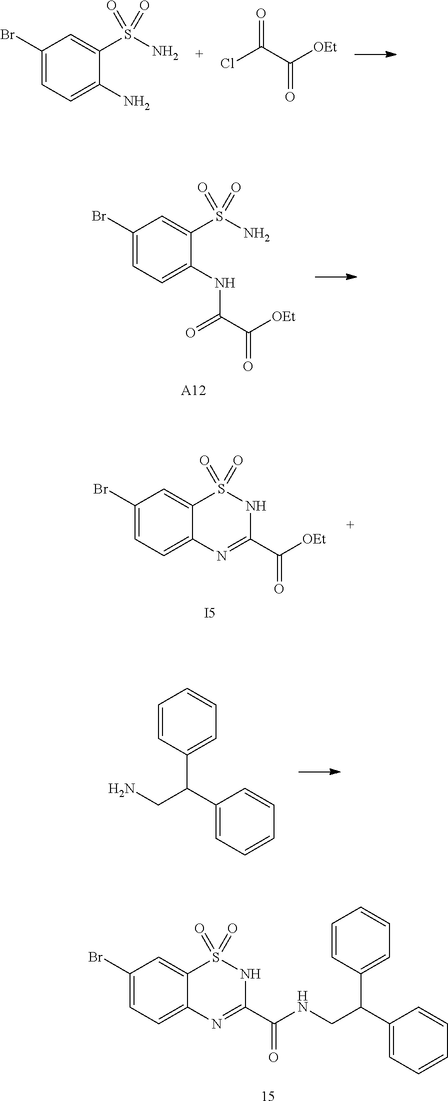

### a) Ethyl 2-((4-bromo-2-sulfamoylphenyl)amino)-2-oxoacetate (A12)

A solution of 2-amino-5-bromobenzenesulfonamide (1.00 g, 3.98 mmol) in anhydrous THF (50 mL) under an atmosphere of nitrogen was cooled in an ice-salt bath. Triethylamine (0.58 mL, 4.2 mmol) was added, followed by the dropwise addition of ethyl chlorooxoacetate (0.47 mL, 4.2 mmol). The mixture was returned to room temperature and stirred for 48 h. The precipitate was removed by filtration and the filtrate was concentrated in vacuo to give the product as a white solid (1.75 g, >100% yield). The crude material was used in the next step without further purification: LCMS-A r.t. 5.95 min; m/z 349.0 [M−H]⁻.

### b) Ethyl 7-bromo-2H-benzo[e][1,2,4]thiadiazine-3-carboxylate 1,1-dioxide (I5—Alternate Synthesis)

Sodium hydride (60% dispersion in mineral oil, 0.191 g, 4.78 mmol) was added to anhydrous EtOH (20 mL) under a nitrogen atmosphere and the mixture was stirred for 10 min. A slurry of ethyl 2-((4-bromo-2-sulfamoylphenyl)amino)-2-oxoacetate (A12) (1.399 g, 3.984 mmol) in anhydrous EtOH (20 mL) was then added and the mixture was stirred for 3 h at room temperature. Water (˜50 mL) was added and the pH was adjusted to ˜3 with aq. HCl (2 M). The mixture was concentrated in vacuo and the precipitate was isolated by filtration. The solid was washed with water and air dried to give the product as a white solid (0.651 g, 49% yield): ¹H NMR (400 MHz, DMSO-d₆) δ 12.88 (s, 1H), 8.06-8.02 (m, 1H), 7.97-7.92 (m, 1H), 7.75-7.70 (m, 1H), 4.44-4.36 (m, 2H), 1.38-1.33 (m, 3H); LCMS-A r.t. 5.83 min; m/z 331/333 [M−H]⁻.

### c) 7-Bromo-N-(2,2-diphenylethyl)-2H-benzo[e][1,2,4]thiadiazine-3-carboxamide 1,1-dioxide (15)

Ethyl 7-bromo-2H-benzo[e][1,2,4]thiadiazine-3-carboxylate 1,1-dioxide (15) (500 mg, 1.50 mmol), 2,2-diphenylethan-1-amine (355 mg, 1.80 mmol) and absolute ethanol (5 mL) were heated in the microwave (100° C./30 min). The mixture was cooled to room temperature, filtered, the collected solids washed with ethanol and air dried to give the product as a white solid (582 mg, 80% yield). LCMS-B rt: 3.52 min; m/z (negative ion) 483.7 [M−H]. ¹H NMR (400 MHz, DMSO-d6) δ 9.24 (t, J=5.9 Hz, 1H), 7.98 (d, J=2.2 Hz, 1H), 7.92 (dd, J=8.9, 2.2 Hz, 1H), 7.74 (d, J=8.9 Hz, 1H), 7.35-7.25 (m, 8H), 7.24-7.14 (m, 2H), 4.48 (t, J=7.9 Hz, 1H), 3.92 (dd, J=7.9, 5.9 Hz, 2H).

### Example 16: N-(2,2-Diphenylethyl)-7-methyl-2H-benzo[e][1,2,4]thiadiazine-3-carboxamide 1,1-dioxide (16)

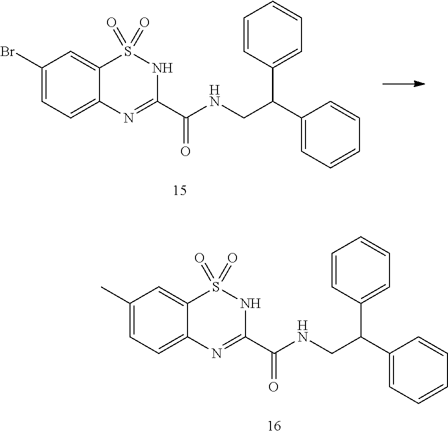

A mixture of 7-bromo-N-(2,2-diphenylethyl)-2H-benzo[e][1,2,4]thiadiazine-3-carboxamide 1,1-dioxide (15) (0.050 g, 0.103 mmol), methylboronic acid (0.012 g, 0.21 mmol) and K₂CO₃ (0.057 g, 0.41 mmol) in dioxane (2 mL) and H₂O (0.5 mL) was bubbled with a stream of nitrogen for 10 min. Pd(dppf)Cl₂.DCM (0.008 g, 0.01 mmol) was then added and the mixture was stirred in the microwave at 100° C. for 30 min. Additional methylboronic acid (0.012 g, 0.21 mmol) and Pd(dppf)Cl₂.DCM (0.008 g, 0.01 mmol) were added, the mixture was bubbled with a stream of nitrogen for 10 min and then stirred in the microwave at 100° C. for 30 min. The volatiles were removed in vacuo before H₂O (5 mL) was added and the aqueous acidified with aq. HCl (2 M). The aqueous phase was extracted with DCM (3×15 mL), the organics were combined, dried (MgSO₄) and the solvent removed in vacuo. The residue was purified by column chromatography (Biotage Isolera, 12 g SiO₂ cartridge, 0-100% EtOAc in petroleum benzine 40-60° C.) to give the product as a white solid (0.013 g, 30% yield): ¹H NMR (400 MHz, DMSO-d₆) δ 12.56 (s, 1H), 9.21 (t, J=5.9, 5.9 Hz, 1H), 7.68 (d, J=8.5 Hz, 1H), 7.65-7.60 (m, 1H), 7.53 (dd, J=8.6, 1.9 Hz, 1H), 7.35-7.26 (m, 8H), 7.23-7.16 (m, 2H), 4.49 (t, J=7.9, 7.9 Hz, 1H), 3.92 (dd, J=7.9, 5.9 Hz, 2H), 2.38 (s, 3H); LCMS-A rt 6.49 min; m/z 418.1 [M−H]⁻.

### Example 17: 7-Cyclopropyl-N-(2,2-diphenylethyl)-2H-benzo[e][1,2,4]thiadiazine-3-carboxamide 1,1-dioxide (17)

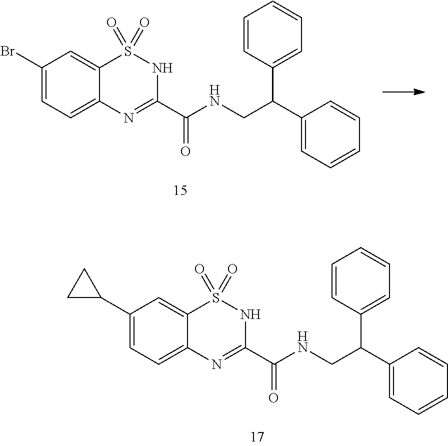 [SMILES: O=C(NCC(c1ccccc1)c1ccccc1)C1=Nc2ccc(Br)cc2S(=O)(=O)N1 ; O=C(NCC(c1ccccc1)c1ccccc1)C1=Nc2ccc(C3CC3)cc2S(=O)(=O)N1]

A mixture of 7-bromo-N-(2,2-diphenylethyl)-2H-benzo[e][1,2,4]thiadiazine-3-carboxamide 1,1-dioxide (15) (0.050 g, 0.103 mmol), cyclopropyl boronic acid (0.018 g, 0.21 mmol) and K₂OO₃ (0.057 g, 0.41 mmol) in dioxane (2 mL) and H₂O (0.5 mL) was bubbled with a stream of nitrogen for 10 min. Pd(dppf)Cl₂.DCM (0.008 g, 0.01 mmol) was then added and the mixture was stirred in the microwave at 100° C. for 60 min. Additional cyclopropyl boronic acid (0.018 g, 0.21 mmol) and Pd(dppf)Cl₂.DCM (0.008 g, 0.01 mmol) were added and the reaction mixture was bubbled with a stream of nitrogen for 10 min before heating in the microwave at 100° C. for 60 min. Further cyclopropyl boronic acid (0.036 g, 0.42 mmol) and Pd(dppf)Cl₂.DCM (0.008 g, 0.01 mmol) were added and the reaction mixture was bubbled with a stream of nitrogen for 10 min before heating in the microwave at 110° C. for 60 min. The volatiles were removed in vacuo before H₂O (5 mL) was added and the aqueous phase acidified with aq. HCl (2 M). The aqueous phase was extracted with DCM (3×15 mL), the organics combined, dried (MgSO₄) and the solvent removed in vacuo. The residue was purified by column chromatography (Biotage Isolera, 12 g SiO₂ cartridge, 0-100% EtOAc in petroleum benzine 40-60° C.) to give the product as a white solid (0.015 g, 33% yield): ¹H NMR (400 MHz, DMSO-d₆) δ 12.56 (s, 1H), 9.25-9.13 (m, 1H), 7.67 (d, J=8.7 Hz, 1H), 7.53 (d, J=2.0 Hz, 1H), 7.38 (dd, J=8.7, 2.1 Hz, 1H), 7.33-7.27 (m, 8H), 7.22-7.16 (m, 2H), 4.48 (t, J=7.9, 7.9 Hz, 1H), 3.92 (dd, J=7.9, 5.9 Hz, 2H), 2.14-2.03 (m, 1H), 1.08-0.94 (m, 2H), 0.79-0.68 (m, 2H); LCMS-B rt 3.45 min; m/z 446.1 [M+H]⁺.

### Example 18: N-(2,2-diphenylethyl)-7-(1-methyl-1H-pyrazol-4-yl)-2H-benzo[e][1,2,4]thiadiazine-3-carboxamide 1,1-dioxide (18)

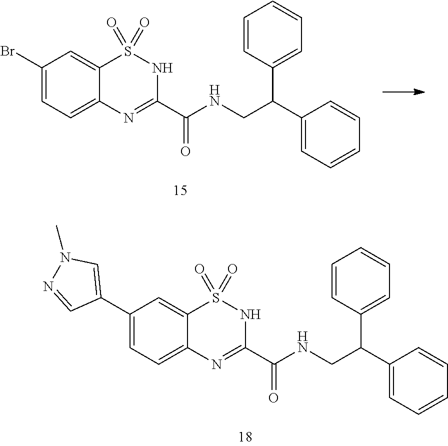 [SMILES: O=C(NCC(c1ccccc1)c1ccccc1)C1=Nc2ccc(Br)cc2S(=O)(=O)N1 ; Cn1cc(-c2ccc3c(c2)S(=O)(=O)NC(C(=O)NCC(c2ccccc2)c2ccccc2)=N3)cn1]

A mixture of 7-bromo-N-(2,2-diphenylethyl)-2H-benzo[e][1,2,4]thiadiazine-3-carboxamide 1,1-dioxide (15) (0.050 g, 0.10 mmol), 1-methylpyrazole-4-boronic acid, pinacol ester (0.043 g, 0.21 mmol) and K₂CO₃ (0.057 g, 0.41 mmol) in dioxane (2 mL) and H₂O (0.5 mL) was bubbled with a stream of nitrogen for 10 min. Pd(dppf)Cl₂.DCM (0.008 g, 0.01 mmol) was then added and the mixture was stirred in the microwave at 100° C. for 60 min. The volatiles were removed in vacuo before H₂O (5 mL) was added and the aqueous phase acidified with aq. HCl (2 M). The aqueous layer was extracted with DCM (3×15 mL), the organics combined, dried (MgSO₄) and the solvent removed in vacuo. The residue was purified by column chromatography (Biotage Isolera, 12 g SiO₂ cartridge, 0-100% EtOAc in petroleum benzine 40-60° C.) to give the product as a white solid (0.019 g, 38% yield): ¹H NMR (400 MHz, DMSO-d₆) δ 12.61 (s, 1H), 9.20 (t, J=6.0, 6.0 Hz, 1H), 8.32 (s, 1H), 8.00 (s, 1H), 7.95 (d, J=2.0 Hz, 1H), 7.92 (dd, J=8.6, 2.1 Hz, 1H), 7.76 (d, J=8.7 Hz, 1H), 7.34-7.27 (m, 8H), 7.22-7.17 (m, 2H), 4.49 (t, J=7.9, 7.9 Hz, 1H), 3.93 (dd, J=7.9, 5.9 Hz, 2H), 3.86 (s, 3H); LCMS-B rt 3.34 min; m/z 484.1 [M−H]⁻.

### Example 19: N-(2,2-diphenylethyl)-7-methoxy-2H-benzo[e][1,2,4]thiadiazine-3-carboxamide 1,1-dioxide (19)

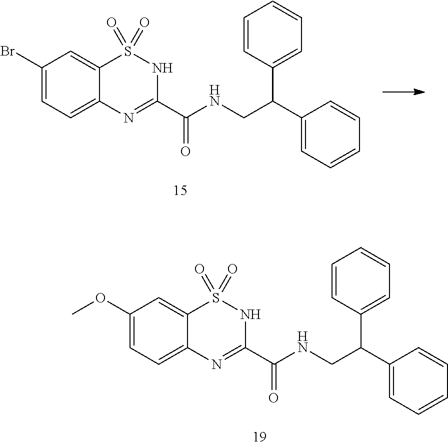 [SMILES: O=C(NCC(c1ccccc1)c1ccccc1)C1=Nc2ccc(Br)cc2S(=O)(=O)N1 ; COc1ccc2c(c1)S(=O)(=O)NC(C(=O)NCC(c1ccccc1)c1ccccc1)=N2]

A mixture of 7-bromo-N-(2,2-diphenylethyl)-2H-benzo[e][1,2,4]thiadiazine-3-carboxamide 1,1-dioxide (15) (0.050 g, 0.10 mmol), Cs₂CO₃ (0.135 g, 0.413 mmol), 1,10-phenanthroline (0.007 g, 0.04 mmol) and CuI (0.008 g, 0.04 mmol) in MeOH (2 mL) was stirred under an atmosphere of nitrogen at 110° C. overnight. The reaction mixture was cooled to room temperature before sodium hydride (60% dispersion in mineral oil, 0.017 g, 0.41 mmol) was added. The mixture was heated at 110° C. overnight under an atmosphere of nitrogen. The reaction mixture was returned to room temperature and additional sodium hydride (60% dispersion in mineral oil, 0.017 g, 0.41 mmol) and CuI (0.008 g, 0.04 mmol) were added. The mixture was heated at 120° C. under an atmosphere of nitrogen for 72 h. The mixture was cooled to room temperature, water (10 mL) and aq. HCl (2 M, 10 mL) were added and the aqueous was extracted with DCM (3×15 mL). The organics were combined, dried (MgSO₄), the solvent removed in vacuo and the solid purified by column chromatography (Biotage Isolera, 12 g SiO₂ cartridge, 0-100% EtOAc in petroleum benzine 40-60° C.) to give the product as a white solid (0.009 g, 20% yield): ¹H NMR (400 MHz, DMSO-d₆) δ 12.59 (s, 1H), 9.20 (t, J=6.0 Hz, 1H), 7.76 (d, J=9.3 Hz, 1H), 7.37-7.28 (m, 9H), 7.25-7.17 (m, 3H), 4.49 (t, J=7.9 Hz, 1H), 3.96-3.89 (m, 2H), 3.84 (s, 3H); LCMS-B rt 3.35 min; m/z 436.1 [M+H]⁺.

### Example 20: Methyl 3-(2,2-diphenylethyl)carbamoyl)-2H-benzo[e][1,2,4]thiadiazine-7-carboxylate 1,1-dioxide (20)

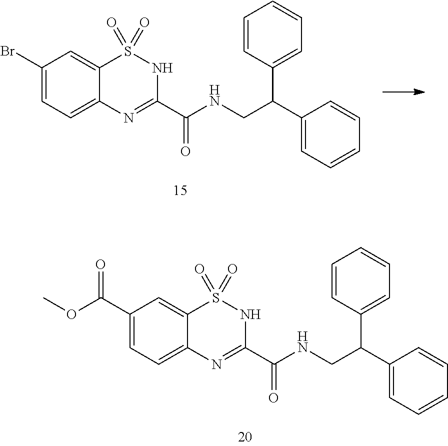 [SMILES: O=C(NCC(c1ccccc1)c1ccccc1)C1=Nc2ccc(Br)cc2S(=O)(=O)N1 ; COC(=O)c1ccc2c(c1)S(=O)(=O)NC(C(=O)NCC(c1ccccc1)c1ccccc1)=N2]

A mixture of 7-bromo-N-(2,2-diphenylethyl)-2H-benzo[e][1,2,4]thiadiazine-3-carboxamide 1,1-dioxide (15) (0.120 g, 0.248 mmol), PdCl₂(dppf).DCM (0.020 g, 0.025 mmol), triethylamine (0.14 mL, 0.99 mmol) and MeOH (3 mL) was loaded into a Schlenk tube under an atmosphere of nitrogen. The tube was flushed with carbon monoxide and the mixture was stirred overnight at 110° C. Additional PdCl₂(dppf).DCM (0.020 g, 0.025 mmol) and triethylamine (1.0 mL, 7.2 mmol) were added and the mixture was stirred at 120° C. for 24 h under an atmosphere of carbon monoxide. The mixture was cooled to room temperature and the volatiles were removed in vacuo. Water (10 mL) and aq. HCl (2 M, 10 mL) were added and the aqueous was extracted with DCM (3×20 mL). The organics were combined, dried (MgSO₄) and the solvent removed in vacuo. The resultant residue was purified by column chromatography (Biotage Isolera, 24 g SiO₂ cartridge, 0-100% EtOAc in petroleum benzine 40-60° C.) to give the product (˜80% purity, 0.064 g, 45% yield) as an off-white solid: ¹H NMR (400 MHz, DMSO-d₆) δ 12.91 (s, 1H), 9.37-9.27 (m, 1H), 8.26 (d, J=1.9 Hz, 1H), 8.24-8.19 (m, 1H), 7.89 (d, J=8.8 Hz, 1H), 7.33-7.28 (m, 8H), 7.22-7.18 (m, 2H), 4.49 (t, J=7.9 Hz, 1H), 3.96-3.90 (m, 2H), 3.89 (s, 3H); LCMS-B rt 3.39 min; m/z 464.1 [M+H]⁺.

### Example 21: 3-(2,2-Diphenylethyl)carbamoyl)-2H-benzo[e][1,2,4]thiadiazine-7-carboxylic Acid 1,1-dioxide (21)

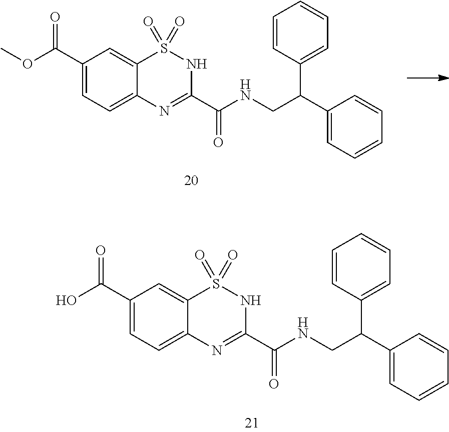 [SMILES: COC(=O)c1ccc2c(c1)S(=O)(=O)NC(C(=O)NCC(c1ccccc1)c1ccccc1)=N2 ; O=C(NCC(c1ccccc1)c1ccccc1)C1=Nc2ccc(C(=O)O)cc2S(=O)(=O)N1]

A mixture of methyl 3-((2,2-diphenylethyl)carbamoyl)-2H-benzo[e][1,2,4]thiadiazine-7-carboxylate 1,1-dioxide (20) (˜80% purity, 0.061 g, 0.11 mmol), LiOH.H₂O (0.044 g, 1.1 mmol), THF (3.5 mL), MeOH (3.5 mL) and H₂O (0.75 mL) were stirred at room temperature overnight. The mixture was concentrated in vacuo before H₂O (5 mL) and aq. HCl (2 M, 5 mL) were added. The aqueous phase was extracted with EtOAc (3×20 mL), the organics were combined, washed with brine and dried (MgSO₄). The solvent was removed in vacuo and the solid was purified by column chromatography (Biotage Isolera, 12 g SiO₂ cartridge, 0-100% EtOAc in petroleum benzine 40-60° C.) to give the product as a white solid (0.016 g, 34% yield): ¹H NMR (400 MHz, DMSO-d₆) δ 13.49 (s, 1H), 12.88 (s, 1H), 9.36-9.24 (m, 1H), 8.27-8.23 (m, 1H), 8.20 (d, J=8.6 Hz, 1H), 7.88 (d, J=8.7 Hz, 1H), 7.35-7.27 (m, 8H), 7.23-7.16 (m, 2H), 4.49 (t, J=7.9 Hz, 1H), 3.97-3.89 (m, 2H); LCMS-B rt 3.29 min; m/z 450.1 [M+H]⁺.

### Example 22: N-(2,2-diphenylethyl)-7-(1H-pyrazol-5-yl)-2H-benzo[e][1,2,4]thiadiazine-3-carboxamide 1,1-dioxide (22)

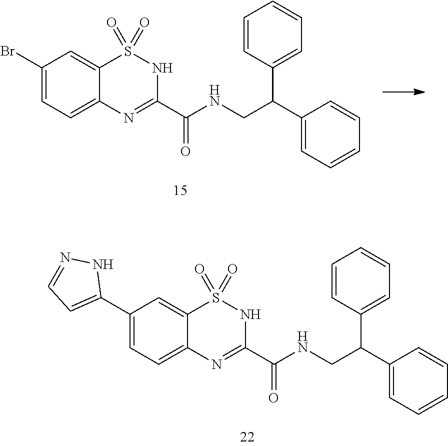 [SMILES: O=C(NCC(c1ccccc1)c1ccccc1)C1=Nc2ccc(Br)cc2S(=O)(=O)N1 ; O=C(NCC(c1ccccc1)c1ccccc1)C1=Nc2ccc(-c3ccn[nH]3)cc2S(=O)(=O)N1]

A mixture of 7-bromo-N-(2,2-diphenylethyl)-2H-benzo[e][1,2,4]thiadiazine-3-carboxamide 1,1-dioxide (15) (0.040 g, 0.083 mmol), (1H-pyrazol-5-yl)boronic acid (0.018 g, 0.17 mmol), and K₂CO₃ (0.046 g, 0.33 mmol) in dioxane (2 mL) and H₂O (0.5 mL) was bubbled with a stream of nitrogen for 5 min. PdCl₂(dppf).DCM (0.007 g, 0.008 mmol) was then added and the mixture was stirred in the microwave at 100° C. for 60 min. The volatiles were removed in vacuo, H₂O (5 mL) was added and the pH of the aqueous was adjusted to ˜3. The aqueous phase was extracted with DCM (3×10 mL), the organics were combined, dried (MgSO₄) and concentrated in vacuo. The solid residue was purified by column chromatography (Biotage Isolera, 12 g SiO₂ cartridge, 0-100% EtOAc in petroleum benzine 40-60° C.) to give the product as a white solid (˜85% purity, 0.004 g, 9% yield): ¹H NMR (400 MHz, DMSO-d₆) δ 13.08 (s, 1H), 12.68 (s, 1H), 9.24 (t, J=6.0 Hz, 1H), 8.22-8.12 (m, 2H), 7.88-7.77 (m, 2H), 7.36-7.26 (m, 8H), 7.24-7.15 (m, 2H), 6.92-6.82 (m, 1H), 4.50 (t, J=7.9 Hz, 1H), 3.93 (dd, J=7.9, 5.8 Hz, 2H); LCMS-B rt 3.31 min; m/z 472.1 [M+H]⁺.

### Example 23: Methyl 3-((2-(oxazol-2-yl)-2-phenylethyl)carbamoyl)-2H-benzo[e][1,2,4]thiadiazine-7-carboxylate 1,1-dioxide (23)

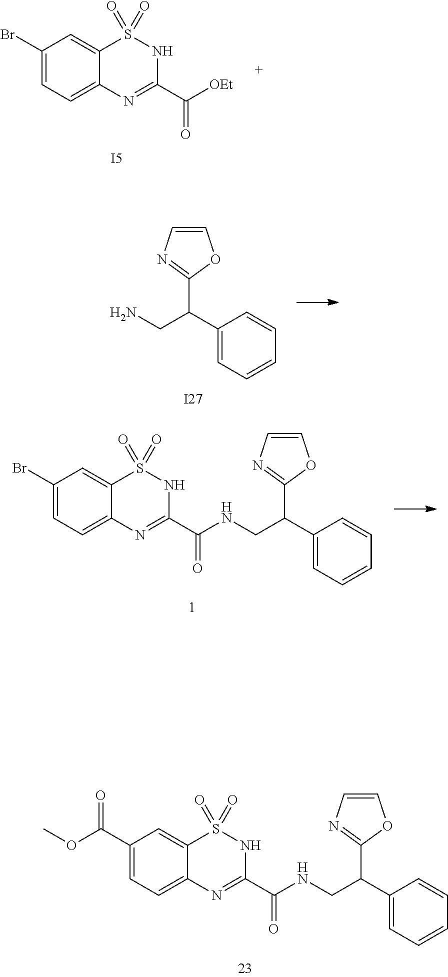

### a) 7-Bromo-N-(2-(oxazol-2-yl)-2-phenylethyl)-2H-benzo[e][1,2,4]thiadiazine-3-carboxamide 1,1-dioxide (1)—Further Synthesis

A mixture of ethyl 7-bromo-2H-benzo[e][1,2,4]thiadiazine-3-carboxylate 1,1-dioxide (I5) (90% purity, 1.96 g, 5.31 mmol), 2-(oxazol-2-yl)-2-phenylethan-1-amine (I27) (0.951 g, 5.05 mmol) and EtOH (4 mL) was heated in the microwave at 100° C. for 60 min and then 110° C. for 30 min. To encourage consumption of starting material, the mixture was stirred in the microwave at 110° C. for a further 60 min and then 120° C. for 30 min. The white precipitate was isolated by vacuum filtration, washed with EtOH and air dried to give a mixture of the desired product and starting material. The solid was taken up in THF (10 mL), MeOH (1 mL) and H₂O (1 mL) and stirred with LiOH.H₂O (0.300 g, 7.15 mmol) for 4 h at room temperature. The mixture was concentrated in vacuo, water (˜50 mL) and aq. HCl (2 M, −50 mL) were added and the mixture sonicated for 10 min. The white precipitate was isolated by vacuum filtration, washed with H₂O and air dried to give the product as a white solid (1.38 g, 57% yield): ¹H NMR (400 MHz, DMSO-d₆) δ 12.87-12.63 (s, 1H), 9.36-9.24 (t, J=5.9 Hz, 1H), 8.06-8.03 (m, 1H), 8.02-7.99 (d, J=2.2 Hz, 1H), 7.96-7.91 (dd, J=8.9, 2.2 Hz, 1H), 7.78-7.72 (d, J=8.9 Hz, 1H), 7.37-7.31 (m, 2H), 7.30-7.23 (m, 3H), 7.22-7.17 (m, 1H), 4.73-4.61 (t, J=7.6 Hz, 1H), 4.08-3.95 (m, 1H), 3.93-3.81 (m, 1H); LCMS-A rt 6.33 min; m/z 475/477 [M+H]⁺.

### b) Methyl 3-((2-(oxazol-2-yl)-2-phenylethyl)carbamoyl)-2H-benzo[e][1,2,4]thiadiazine-7-carboxylate 1,1-dioxide (23)

A mixture of 7-bromo-N-(2-(oxazol-2-yl)-2-phenylethyl)-2H-benzo[e][1,2,4]thiadiazine-3-carboxamide 1,1-dioxide (1) (0.100 g, 0.210 mmol) and Pd(dppf)Cl₂.DCM (0.052 g, 0.063 mmol) in MeOH (2 mL) was bubbled with CO for 10 min. Triethylamine (2 mL) was added and the mixture was stirred at 120° C. under a balloon of CO for 16 h. Additional Pd(dppf)Cl₂.DCM (0.052 g, 0.063 mmol) was added and the mixture was stirred at 120° C. under a balloon of CO for 4 h. The mixture was cooled to room temperature and concentrated in vacuo. Water (˜15 mL) was added and the aqueous phase was brought to pH ˜2 with aq. HCl (2 M). The aqueous layer was extracted with DCM (3×30 mL), the organics were combined, washed with brine, dried (MgSO₄), the solvent removed in vacuo and the residue purified by column chromatography (Biotage Isolera, 24 g SiO₂ cartridge, 0-100% EtOAc in petroleum benzine 40-60° C.) to give the product as an orange solid (0.035 g, 37% yield): ¹H NMR (400 MHz, DMSO-d₆) δ 12.92 (s, 1H), 9.36-9.29 (t, J=6.2 Hz, 1H), 8.31-8.25 (d, J=1.9 Hz, 1H), 8.25-8.19 (dd, J=8.7, 1.9 Hz, 1H), 8.07-8.02 (d, J=0.9 Hz, 1H), 7.93-7.86 (d, J=8.7 Hz, 1H), 7.37-7.31 (m, 2H), 7.30-7.25 (m, 3H), 7.22-7.18 (d, J=0.9 Hz, 1H), 4.72-4.63 (t, J=7.6 Hz, 1H), 4.05-3.97 (m, 1H), 3.92-3.85 (m, 4H); LCMS-A rt 6.26 min; m/z 455.1 [M+H]⁺.

### Example 24: 3-((2-(Oxazol-2-yl)-2-phenylethyl)carbamoyl)-2H-benzo[e][1,2,4]thiadiazine-7-carboxylic Acid 1,1-dioxide (24)

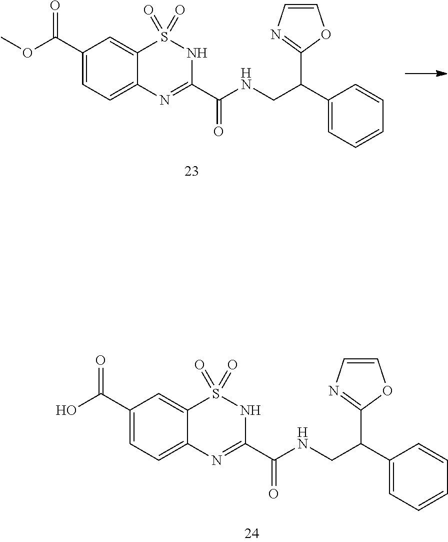 [SMILES: COC(=O)c1ccc2c(c1)S(=O)(=O)NC(C(=O)NCC(c1ccccc1)c1ncco1)=N2 ; O=C(NCC(c1ccccc1)c1ncco1)C1=Nc2ccc(C(=O)O)cc2S(=O)(=O)N1]

A mixture of methyl 3-((2-(oxazol-2-yl)-2-phenylethyl)carbamoyl)-2H-benzo[e][1,2,4]thiadiazine-7-carboxylate 1,1-dioxide (23) (0.060 g, 0.13 mmol), LiOH.H₂O (0.028 g, 0.66 mmol), THF (3.5 mL), MeOH (3.5 mL) and H₂O (0.75 mL) was stirred at room temperature for 18 h. Additional LiOH.H₂O (0.028 g, 0.66 mmol) was added and the mixture was stirred at room temperature for 4 h. Another portion of LiOH.H₂O (0.028 g, 0.66 mmol) was added and the mixture was stirred at 40° C. for 1.5 h. The volatiles were removed in vacuo, H₂O (˜20 mL) was added and the aqueous layer was washed with DCM (2×20 mL). The aqueous phase was adjusted to pH ˜2 with aq. HCl (2 M) and then extracted with DCM (3×20 mL). The organics were combined, washed with brine, dried (Na₂SO₄), the solvent was removed in vacuo and the residue was purified by column chromatography (Biotage Isolera, 4 g SiO₂ cartridge, 0-100% EtOAc in petroleum benzine 40-60° C.). The fraction containing the suspected product was purified by another round of column chromatography (Biotage Isolera, 4 g SiO₂ cartridge, 0-5% MeOH in DCM) to give the product as a white solid (0.007 g, 12% yield): ¹H NMR (400 MHz, DMSO-d₆) δ 12.92 (s, 1H), 9.40-9.27 (t, J=5.9 Hz, 1H), 8.30-8.24 (d, J=1.8 Hz, 1H), 8.23-8.17 (dd, J=8.9, 1.9 Hz, 1H), 8.08-8.01 (s, 1H), 7.93-7.83 (d, J=8.7 Hz, 1H), 7.37-7.31 (m, 2H), 7.30-7.23 (m, 3H), 7.24-7.15 (m, 1H), 4.75-4.59 (t, J=7.5 Hz, 1H), 4.08-3.96 (m, 1H), 3.94-3.82 (m, 1H), COOH not observed; LCMS-B RT 3.10 min; m/z 441.0 [M+H]⁺.

### Example 25: 7-(1-Methyl-1H-pyrazol-4-yl)-N-(2-(oxazol-2-yl)-2-phenylethyl)-2H-benzo[e][1,2,4]thiadiazine-3-carboxamide 1,1-dioxide (25)

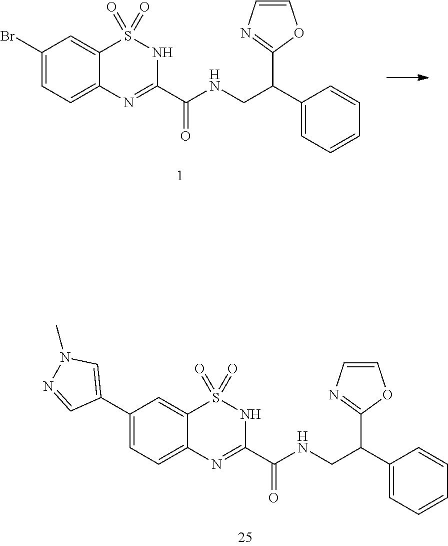 [SMILES: O=C(NCC(c1ccccc1)c1ncco1)C1=Nc2ccc(Br)cc2S(=O)(=O)N1 ; Cn1cc(-c2ccc3c(c2)S(=O)(=O)NC(C(=O)NCC(c2ccccc2)c2ncco2)=N3)cn1]

A mixture of 7-bromo-N-(2-(oxazol-2-yl)-2-phenylethyl)-2H-benzo[e][1,2,4]thiadiazine-3-carboxamide 1,1-dioxide (1) (0.050 g, 0.11 mmol), 1-methyl-1H-pyrazole-4-boronic acid, pinacol ester (0.044 g, 0.21 mmol), Pd(dppf)Cl₂.DCM (0.009 g, 0.01 mmol), H₂O (0.5 mL) and dioxane (2 mL) were bubbled with a stream of nitrogen gas for 10 min. Potassium carbonate (0.058 g, 0.42 mmol) was then added and the mixture was stirred in the microwave at 100° C. for 60 min. The mixture was returned to room temperature and the volatiles were removed in vacuo. Water (˜10 mL) was added and the aqueous phase was adjusted to pH ˜2 with aq. HCl (2 M) and then extracted with DCM (2×15 mL). The organics were combined, the solvent was removed in vacuo and the residue was purified by column chromatography (Biotage Isolera, 12 g SiO₂ cartridge, 0-100% EtOAc in petroleum benzine 40-60° C.) to give a white solid. The solid was taken up in a minimum amount of DCM, cyclohexane was added and the suspension was sonicated for 5 min. The precipitate was isolated by filtration and air dried to give the product as a white solid (0.011 g, 22% yield): ¹H NMR (400 MHz, DMSO-d₆) δ 12.62 (s, 1H), 9.36-9.15 (t, J=5.9 Hz, 1H), 8.38-8.25 (s, 1H), 8.10-7.95 (m, 3H), 7.95-7.89 (m, 1H), 7.83-7.71 (d, J=8.8 Hz, 1H), 7.37-7.31 (m, 2H), 7.31-7.24 (m, 3H), 7.23-7.18 (s, 1H), 4.75-4.60 (t, J=7.5 Hz, 1H), 4.06-3.95 (m, 1H), 3.94-3.79 (m, 4H); LCMS-B RT 3.15 min; m/z 477.1 [M+H]⁺.

### Example 26: N-(2-(Oxazol-2-yl)-2-phenylethyl)-7-(1H-pyrazol-4-yl)-2H-benzo[e][1, 2, 4]thiadiazine-3-carboxamide 1,1-dioxide (26)

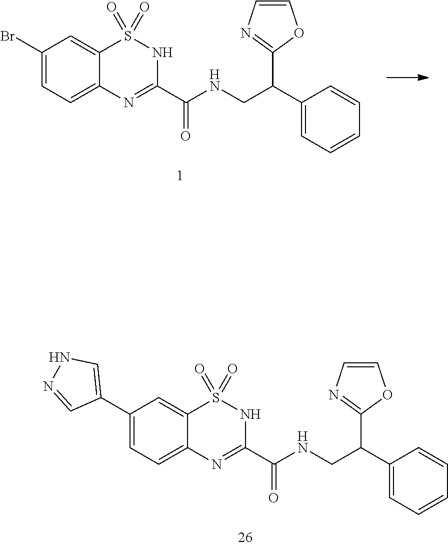 [SMILES: O=C(NCC(c1ccccc1)c1ncco1)C1=Nc2ccc(Br)cc2S(=O)(=O)N1 ; O=C(NCC(c1ccccc1)c1ncco1)C1=Nc2ccc(-c3cn[nH]c3)cc2S(=O)(=O)N1]

A mixture of 7-bromo-N-(2-(oxazol-2-yl)-2-phenylethyl)-2H-benzo[e][1,2,4]thiadiazine-3-carboxamide 1,1-dioxide (1) (0.050 g, 0.11 mmol), pyrazole-4-boronic acid (HCl salt, 0.031 g, 0.21 mmol), Pd(dppf)Cl₂.DCM (0.009 g, 0.01 mmol), H₂O (0.5 mL) and dioxane (2 mL) were bubbled with a stream of nitrogen gas for 10 min. Potassium carbonate (0.058 g, 0.42 mmol) was then added and the mixture was stirred in the microwave at 100° C. for 60 min. The mixture was returned to room temperature and the volatiles were removed in vacuo. Water (˜10 mL) was added and the aqueous was adjusted to pH ˜2 with aq. HCl (2 M) and then extracted with DCM (2×15 mL). The organics were combined, the solvent was removed in vacuo and the residue was purified by column chromatography (Biotage Isolera, 12 g SiO₂ cartridge, 0-100% EtOAc in petroleum benzine 40-60° C.) to give the product as a white solid (0.010 g, 21% yield): ¹H NMR (400 MHz, DMSO-d₆) δ 13.07 (s, 1H), 12.68-12.49 (s, 1H), 9.34-9.11 (m, 1H), 8.54-8.22 (s, 1H), 8.20-8.01 (m, 3H), 8.00-7.94 (dd, J=8.6, 2.1 Hz, 1H), 7.83-7.71 (d, J=8.6 Hz, 1H), 7.38-7.31 (m, 2H), 7.31-7.24 (m, 3H), 7.23-7.17 (m, 1H), 4.72-4.63 (t, J=7.5 Hz, 1H), 4.06-3.96 (m, 1H), 3.94-3.83 (m, 1H); LCMS-A RT 5.49 min; m/z 463.2 [M+H]⁺.

### Example 27: N³-(2-(Oxazol-2-yl)-2-phenylethyl)-2H-benzo[e][1,2,4]thiadiazine-3,7-dicarboxamide 1,1-dioxide (27)

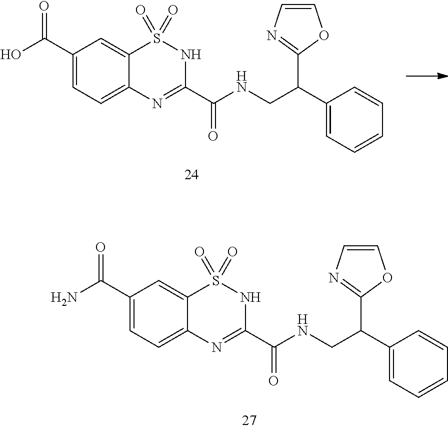 [SMILES: O=C(NCC(c1ccccc1)c1ncco1)C1=Nc2ccc(C(=O)O)cc2S(=O)(=O)N1 ; NC(=O)c1ccc2c(c1)S(=O)(=O)NC(C(=O)NCC(c1ccccc1)c1ncco1)=N2]

DIPEA (79 μL, 0.45 mmol) was added to a solution of 3-((2-(oxazol-2-yl)-2-phenylethyl)carbamoyl)-2H-benzo[e][1,2,4]thiadiazine-7-carboxylic acid 1,1-dioxide (24) (0.040 g, 0.091 mmol) in THF (3 mL) and DMF (0.5 mL). HOBt (0.018 g, 0.14 mmol) and EDCI.HCl (0.026 g, 0.14 mmol) were then added followed by (NH₄)₂CO₃ (0.044 g, 0.45 mmol). The mixture was stirred for 48 h at room temperature before being concentrated in vacuo. Water (˜15 mL) was added and the aqueous was brought to ˜pH 2. The precipitate was isolated by filtration and air dried to give a brown solid. The solid was adsorbed onto silica and purified by column chromatography (Biotage Isolera, 4 g SiO₂ cartridge, 0-100% EtOAc in petroleum benzine 40-60° C.) to give the product as a white solid (0.003 g, 8% yield): ¹H NMR (400 MHz, DMSO-d₆) δ 12.79 (s, 1H), 9.37-9.21 (m, 1H), 8.37 (d, J=2.0 Hz, 1H), 8.25 (s, 1H), 8.17 (dd, J=8.7, 2.0 Hz, 1H), 8.05 (d, J=0.8 Hz, 1H), 7.82 (d, J=8.7 Hz, 1H), 7.60 (s, 1H), 7.38-7.31 (m, 2H), 7.30-7.24 (m, 3H), 7.21 (d, J=0.9 Hz, 1H), 4.67 (t, J=7.5 Hz, 1H), 4.06-3.96 (m, 1H), 3.93-3.83 (m, 1H); LCMS-B RT 3.09 min; m/z 440.1 [M+H]⁺.

### Example 28: N-(2-Bromophenethyl)-2H-benzo[e][1,2,4]thiadiazine-3-carboxamide 1,1-dioxide (28)

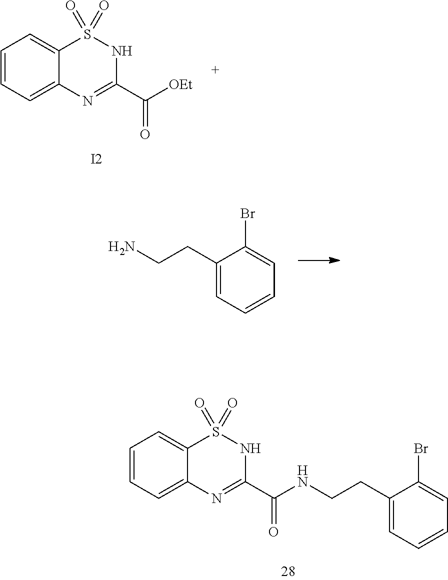 [SMILES: CCOC(=O)C1=Nc2ccccc2S(=O)(=O)N1 ; "I2" ; NCCc1ccccc1Br ; O=C(NCCc1ccccc1Br)C1=Nc2ccccc2S(=O)(=O)N1]

A mixture of ethyl 2H-benzo[e][1,2,4]thiadiazine-3-carboxylate 1,1-dioxide (12) (0.050 g, 0.20 mmol) and 2-(2-bromophenyl)ethan-1-amine (40 μL, 0.28 mmol) in EtOH (0.2 mL) was heated in the microwave at 120° C. for 60 min. The mixture was returned to room temperature and the white precipitate was isolated by filtration, washed with EtOH and air dried to give the product as a white solid (0.057 g, 71% yield): ¹H NMR (400 MHz, DMSO-d₆) δ 12.62 (s, 1H), 9.47-9.25 (t, J=6.0 Hz, 1H), 7.88-7.83 (dd, J=8.0, 1.4 Hz, 1H), 7.83-7.78 (m, 1H), 7.76-7.70 (m, 1H), 7.62-7.57 (m, 1H), 7.56-7.49 (m, 1H), 7.36-7.29 (m, 2H), 7.21-7.13 (m, 1H), 3.59-3.48 (m, 2H), 3.06-2.93 (t, J=7.2 Hz, 2H); LCMS-B RT 3.28 min; m/z 408/410 [M+H]⁺.

### Example 29: N-(2-Hydroxyphenethyl)-2H-benzo[e][1,2,4]thiadiazine-3-carboxamide 1,1-dioxide (29)

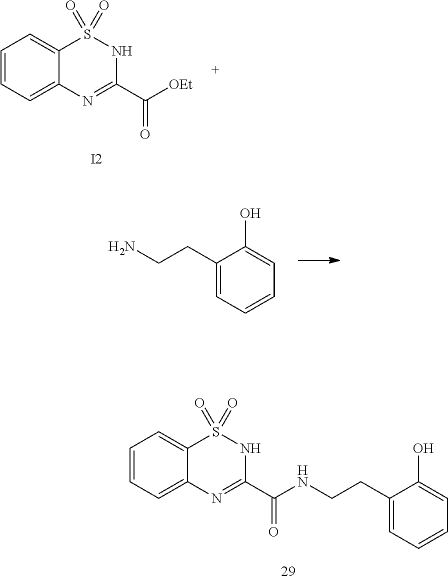 [SMILES: CCOC(=O)C1=Nc2ccccc2S(=O)(=O)N1 ; "I2" ; NCCc1ccccc1O ; O=C(NCCc1ccccc1O)C1=Nc2ccccc2S(=O)(=O)N1]

A mixture of ethyl 2H-benzo[e][1,2,4]thiadiazine-3-carboxylate 1,1-dioxide (12) (0.050 g, 0.20 mmol) and 2-(2-aminoethyl)phenol (0.038 g, 0.28 mmol) in EtOH (0.2 mL) was heated in the microwave at 120° C. for 60 min. The mixture was returned to room temperature and the white precipitate was isolated by filtration, washed with EtOH and air dried to give the product as a white solid (0.031 g, 46% yield): ¹H NMR (400 MHz, DMSO-d₆) δ 12.62 (s, 1H), 9.38 (s, 1H), 9.21 (t, J=5.9 Hz, 1H), 7.86 (dd, J=8.0, 1.4 Hz, 1H), 7.81 (dd, J=8.4, 1.2 Hz, 1H), 7.77-7.69 (m, 1H), 7.56-7.48 (m, 1H), 7.10-6.97 (m, 2H), 6.79 (dd, J=8.1, 1.2 Hz, 1H), 6.71 (td, J=7.4, 1.2 Hz, 1H), 3.54-3.44 (m, 2H), 2.82 (t, J=7.3 Hz, 2H); LCMS-A RT 6.07 min; m/z 344.1 [M−H]⁻.

### Example 30: 2-(2-(1,1-Dioxido-2H-benzo[e][1,2,4]thiadiazine-3-carboxamido)ethyl)benzoic Acid (30)

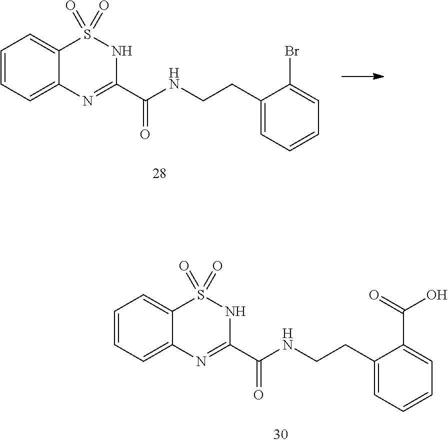 [SMILES: O=C(NCCc1ccccc1Br)C1=Nc2ccccc2S(=O)(=O)N1 ; O=C(NCCc1ccccc1C(=O)O)C1=Nc2ccccc2S(=O)(=O)N1]

A solution of N-(2-bromophenethyl)-2H-benzo[e][1,2,4]thiadiazine-3-carboxamide 1,1-dioxide (28) (0.200 g, 0.490 mmol) in anhydrous THF (2 mL) was cooled to −78° C. under an atmosphere of nitrogen. A solution of n-butyllithium (1.6 M in hexanes, 0.64 mL, 1.0 mmol) was cautiously added and the mixture was stirred for 10 min at −78° C. The mixture was then poured onto dry ice and returned to room temperature with stirring. Water was added (˜10 mL) and the mixture was concentrated in vacuo. The aqueous was adjusted to pH ˜2 with aq. HCl (2 M) and then extracted with DCM (2×15 mL). The organics were combined, washed with brine, dried (Na₂SO₄) and the solvent removed in vacuo. The white solid was purified by column chromatography (Biotage Isolera, 24 g SiO₂ cartridge, 0-100% EtOAc in petroleum benzine 40-60° C. then 0-25% MeOH in EtOAc) to give the product as a white solid (0.019 g, 10% yield): ¹H NMR (400 MHz, DMSO-d₆) δ 9.24 (t, J=5.8 Hz, 1H), 7.87-7.81 (m, 2H), 7.78 (d, J=8.4 Hz, 1H), 7.74-7.68 (m, 1H), 7.55-7.43 (m, 2H), 7.35-7.28 (m, 2H), 3.56 (q, J=6.6 Hz, 2H), 3.22 (t, J=7.0 Hz, 2H), CO₂H and SO₂NH not observed; LCMS-B RT 3.09 min; m/z 372.0 [M−H]⁻.

### Example 31: N-(2-Iodophenethyl)-2H-benzo[e][1,2,4]thiadiazine-3-carboxamide 1,1-dioxide (31)

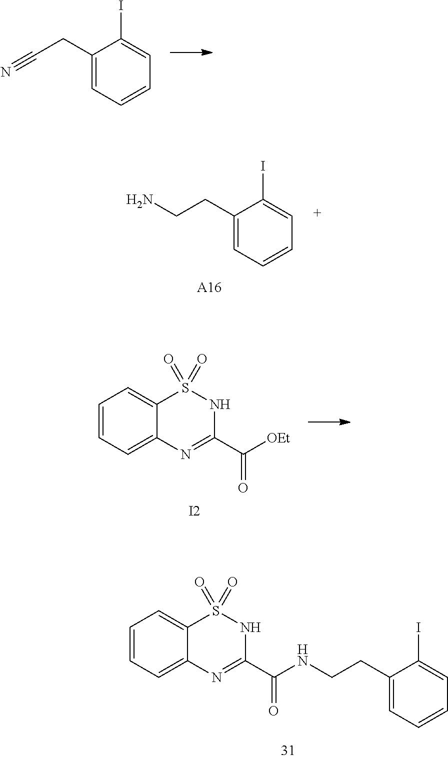 [SMILES: N#CCc1ccccc1I ; NCCc1ccccc1I ; "A16" ; CCOC(=O)C1=Nc2ccccc2S(=O)(=O)N1 ; "I2" ; O=C(NCCc1ccccc1I)C1=Nc2ccccc2S(=O)(=O)N1]

### a) 2-(2-Iodophenyl)ethan-1-amine (A16)

A solution of 2-(2-iodophenyl)acetonitrile (1.00 g, 4.11 mmol) in anhydrous THF (5 mL) under an atmosphere of nitrogen was treated with borane tetrahydrofuran complex solution (1.0 M in THF, 12.3 mL, 12.3 mmol). The mixture was stirred at reflux for 16 h, cooled to room temperature and excess borane reagent was quenched by the dropwise addition of water (until evolution of hydrogen ceased). MeOH (2.5 mL) and conc. H₂SO₄ (0.5 mL) was added and the mixture was stirred for 1 h at r.t. The mixture was concentrated in vacuo, water (˜10 mL) was added and the aqueous was adjusted to pH ˜12 with aq. NaOH (2 M). The aqueous layer was extracted with EtOAc (3×30 mL), the organics were combined, washed with brine, dried (Na₂SO₄) and the solvent removed in vacuo to give a colourless oil. Water (˜20 mL) was added and the aqueous phase was adjusted to pH ˜2 with aq. HCl (2 M). The aqueous layer was washed with DCM (3×30 mL) and then adjusted to pH ˜12 with aq. NaOH (2 M). The aqueous layer was extracted with DCM (3×50 mL), the organics were combined, washed with brine, dried (Na₂SO₄) and the solvent removed in vacuo to give the product as a colourless oil (0.869 g, 85% yield): ¹H NMR (400 MHz, DMSO-d₆) δ 7.81 (dd, J=7.8, 1.2 Hz, 1H), 7.35-7.27 (m, 2H), 6.97-6.91 (m, 1H), 2.75-2.71 (m, 4H) exchangeable NH not observed; LCMS-B RT 2.77 min; m/z 248.0 [M+H]⁺.

### b) N-(2-Iodophenethyl)-2H-benzo[e][1,2,4]thiadiazine-3-carboxamide 1,1-dioxide (31)

A mixture of ethyl 2H-benzo[e][1,2,4]thiadiazine-3-carboxylate 1,1-dioxide (12) (0.250 g, 0.983 mmol) and A16 (0.340 g, 1.38 mmol) in EtOH (1 mL) was heated in the microwave at 120° C. for 60 min. The mixture was returned to room temperature and the white precipitate was isolated by filtration, washed with EtOH and air dried to give the title compound as a white solid (0.370 g, 83% yield): ¹H NMR (400 MHz, DMSO-d₆) δ 12.62 (s, 1H), 9.36 (t, J=5.8 Hz, 1H), 7.88-7.82 (m, 2H), 7.80 (dd, J=8.4, 1.2 Hz, 1H), 7.76-7.70 (m, 1H), 7.56-7.49 (m, 1H), 7.37-7.28 (m, 2H), 7.01-6.94 (m, 1H), 3.55-3.46 (m, 2H), 2.98 (t, J=7.3 Hz, 2H); LCMS-B RT 3.32 min; m/z 455.9 [M+H]⁺.

### Example 32: N⁷-Methyl-N³-(2-(oxazol-2-yl)-2-phenylethyl)-2H-benzo[e][1,2,4]thiadiazine-3,7-dicarboxamide 1,1-dioxide (32)

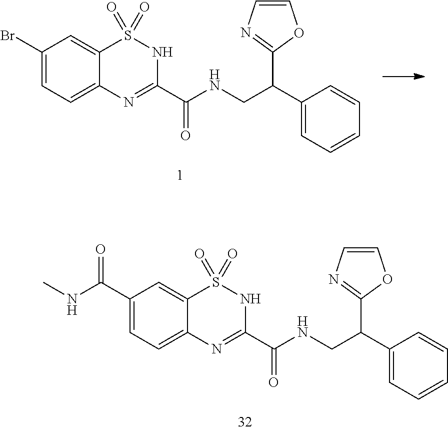 [SMILES: O=C(NCC(c1ccccc1)c1ncco1)C1=Nc2ccc(Br)cc2S(=O)(=O)N1 ; CNC(=O)c1ccc2c(c1)S(=O)(=O)NC(C(=O)NCC(c1ccccc1)c1ncco1)=N2]

A mixture of 7-bromo-N-(2-(oxazol-2-yl)-2-phenylethyl)-2H-benzo[e][1,2,4]thiadiazine-3-carboxamide 1,1-dioxide (1) (0.050 g, 0.11 mmol), methylamine hydrochloride (0.036 g, 0.53 mmol), Pd(OAc)₂ (0.002 g, 0.009 mmol) and xantphos (0.004 g, 0.007 mmol) in 1,4-dioxane (3 mL) and triethylamine (0.15 mL, 1.1 mmol) was bubbled with CO_{(g)} for 10 min. The mixture was then refluxed under a balloon of CO for 16 h. Additional portions of methylamine hydrochloride (0.036 g, 0.53 mmol), Pd(OAc)₂ (0.002 g, 0.009 mmol), xantphos (0.004 g, 0.007 mmol) and triethylamine (0.15 mL, 1.1 mmol) were added and the mixture was stirred at reflux for a further 24 h under a balloon of CO. The mixture was returned to room temperature and then concentrated in vacuo. Water (˜10 mL) was added to the residue and the pH was adjusted to ˜2 with aq. HCl (2 M). The aqueous was extracted with EtOAc (3×15 mL), the organics were combined, washed with brine and dried (Na₂SO₄). The solvent was removed in vacuo and the residue was purified by column chromatography (Biotage Isolera, 12 g SiO₂ cartridge, 0-100% EtOAc in petroleum benzine 40-60° C.) to give the product as a white solid (0.014 g, 29% yield): ¹H NMR (400 MHz, DMSO-d₆) δ 12.79 (s, 1H), 9.37-9.19 (m, 1H), 8.78-8.67 (m, 1H), 8.32 (d, J=2.0 Hz, 1H), 8.13 (dd, J=8.7, 2.0 Hz, 1H), 8.05 (d, J=0.9 Hz, 1H), 7.83 (d, J=8.7 Hz, 1H), 7.37-7.31 (m, 2H), 7.30-7.24 (m, 3H), 7.21 (d, J=1.0 Hz, 1H), 4.67 (t, J=7.5 Hz, 1H), 4.07-3.96 (m, 1H), 3.93-3.83 (m, 1H), 2.80 (d, J=4.5 Hz, 3H); LCMS-B RT 3.10 min; m/z 454.1 [M+H]⁺.

### Example 33: N⁷,N⁷-Dimethyl-N³-(2-(oxazol-2-yl)-2-phenylethyl)-2H-benzo[e][1,2,4]thiadiazine-3,7-dicarboxamide 1,1-dioxide (33)

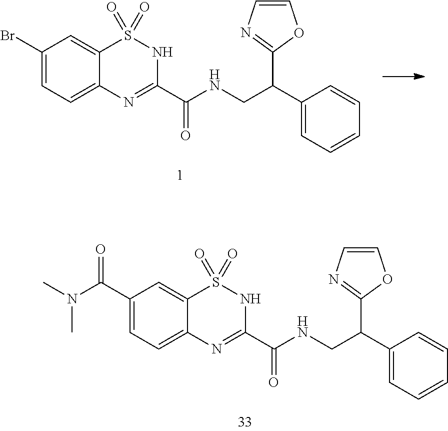 [SMILES: O=C(NCC(c1ccccc1)c1ncco1)C1=Nc2ccc(Br)cc2S(=O)(=O)N1 ; CN(C)C(=O)c1ccc2c(c1)S(=O)(=O)NC(C(=O)NCC(c1ccccc1)c1ncco1)=N2]

A mixture of 7-bromo-N-(2-(oxazol-2-yl)-2-phenylethyl)-2H-benzo[e][1,2,4]thiadiazine-3-carboxamide 1,1-dioxide (1) (0.050 g, 0.11 mmol), dimethylamine hydrochloride (0.043 g, 0.53 mmol), Pd(OAc)₂ (0.002 g, 0.01 mmol) and xantphos (0.006 g, 0.01 mmol) in 1,4-dioxane (3 mL) and triethylamine (0.20 mL, 1.4 mmol) was bubbled with CO_{(g)} for 10 min. The mixture was then refluxed under a balloon of CO for 16 h. Additional portions of dimethylamine hydrochloride (0.043 g, 0.53 mmol), Pd(OAc)₂ (0.002 g, 0.01 mmol), xantphos (0.006 g, 0.01 mmol) and triethylamine (0.20 mL, 1.4 mmol) were added and the mixture was stirred at reflux for a further 6 h under a balloon of CO. The mixture was returned to room temperature and stirred for 72 h. The mixture was concentrated in vacuo, water (˜10 mL) was added and the pH was adjusted to ˜2 with aq. HCl (2 M). The aqueous layer was extracted with EtOAc (3×15 mL), the organics were combined, washed with brine and dried (Na₂SO₄). The solvent was removed in vacuo and the residue was purified by column chromatography (Biotage Isolera, 12 g SiO₂ cartridge, 0-100% EtOAc in petroleum benzine 40-60° C.) to give the product as a white solid (0.013 g, 26% yield): ¹H NMR (400 MHz, DMSO-d₆) δ 12.77 (s, 1H), 9.44-9.09 (m, 1H), 8.05 (d, J=0.9 Hz, 1H), 7.86-7.72 (m, 3H), 7.37-7.31 (m, 2H), 7.30-7.25 (m, 3H), 7.20 (d, J=0.9 Hz, 1H), 4.67 (t, J=7.5 Hz, 1H), 4.05-3.96 (m, 1H), 3.93-3.83 (m, 1H), 2.95 (d, J=25.7 Hz, 6H); LCMS-B RT 3.09 min; m/z 468.2 [M+H]⁺.

### Example 34: N-(2-(Pyridin-3-yl)phenethyl)-2H-benzo[e][1,2,4]thiadiazine-3-carboxamide

1,1-dioxide (34)

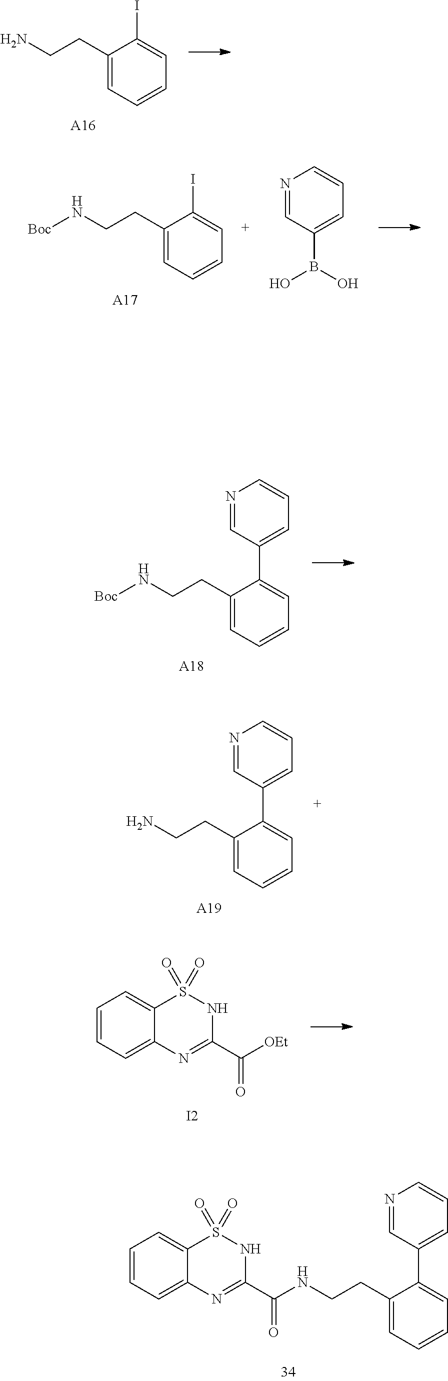

a) tert-Butyl (2-iodophenethyl)carbamate (A17)

A mixture of 2-(2-iodophenyl)ethan-1-amine (A16) (0.432 g, 1.75 mmol), di-tert-butyl dicarbonate (0.458 g, 2.10 mmol), TEA (0.37 mL, 2.6 mmol) and DMAP (0.021 g, 0.18 mmol) in THF (5 mL) was stirred at room temperature for 16 h. Water (˜10 mL) was added and the mixture concentrated in vacuo. The aqueous phase was adjusted to pH ˜2 with aq. HCl (2 M) and then extracted with DCM (3×25 mL). The organics were combined, dried (Na₂SO₄) and the solvent removed in vacuo. The residue was purified by column chromatography (Biotage Isolera, 24 g SiO₂ cartridge, 0-50% EtOAc in petroleum benzine 40-60° C.) to give the product as a white solid (0.506 g, 83% yield): ¹H NMR (400 MHz, DMSO-d₆) δ 7.82 (dd, J=7.9, 1.3 Hz, 1H), 7.33 (td, J=7.4, 1.3 Hz, 1H), 7.29-7.21 (m, 1H), 7.00-6.90 (m, 2H), 3.20-3.08 (m, 2H), 2.83-2.75 (m, 2H), 1.36 (s, 9H); LCMS-B RT 3.50 min; m/z 370.0 [M+Na]⁺, 291.9 [M−t−Bu+2H]⁺.

### b) tert-Butyl (2-(pyridin-3-yl)phenethyl)carbamate (A18)

A mixture of tert-butyl (2-iodophenethyl)carbamate (A17) (0.100 g, 0.288 mmol), pyridine-3-boronic acid (0.071 g, 0.58 mmol), K₂CO₃ (0.119 g, 0.864 mmol) and Pd(dppf)Cl₂.DCM (0.024 g, 0.029 mmol) in 1,4-dioxane (2 mL) and H₂O (0.5 mL) were stirred at reflux under an atmosphere of nitrogen for 3 h. The mixture was cooled to room temperature and then concentrated in vacuo. Water (˜10 mL) and sat. aq. NaHCO₃ (˜10 mL) were added and the aqueous layer was extracted with EtOAc (3×15 mL). The organics were combined, washed with brine, dried (Na₂SO₄), the volatiles evaporated in vacuo and the residue purified by column chromatography (Biotage Isolera, 12 g SiO₂ cartridge, 0-100% EtOAc in petroleum benzine 40-60° C.) to give the product as a colourless oil (0.063 g, 73% yield): ¹H NMR (400 MHz, CDCl₃) δ 8.61 (dd, J=4.9, 1.7 Hz, 1H), 8.57 (dd, J=2.4, 0.9 Hz, 1H), 7.64 (dt, J=7.7, 2.0 Hz, 1H), 7.41-7.27 (m, 4H), 7.21 (dt, J=7.6, 1.0 Hz, 1H), 4.40 (s, 1H), 3.33-3.08 (m, 2H), 2.77 (t, J=7.2 Hz, 2H), 1.39 (s, 9H): LCMS-B rt 3.05 min; m/z 299 [M+H]⁺, 243 [M−t−Bu+2H]⁺.

### c) 2-(2-(Pyridin-3-yl)phenyl)ethan-1-amine (A19)

A solution of tert-butyl (2-(pyridin-3-yl)phenethyl)carbamate (A18) (0.063 g, 0.21 mmol) in DCM (5 mL) was treated with TFA (0.16 mL, 2.1 mmol) and the mixture was stirred at room temperature for 4 h. Another aliquot of TFA (0.16 mL, 2.1 mmol) was added and the mixture was stirred at room temperature for a further 1 hour. Water (˜10 mL) was added, the aqueous phase was adjusted to pH ˜12 with aq. NaOH (2 M) and then extracted with EtOAc (3×20 mL). The organics were combined, washed with brine, dried (Na₂SO₄) and the solvent removed in vacuo to give the product as a colourless oil (0.043 g, >95% yield): ¹H NMR (400 MHz, CDCl₃) δ 8.58-8.52 (m, 2H), 7.64 (dt, J=7.7, 2.0 Hz, 1H), 7.39-7.27 (m, 4H), 7.20 (dd, J=7.4, 1.4 Hz, 1H), 2.87-2.73 (m, 6H); LCMS-B RT 0.50 min; m/z 199.1 [M+H]⁺.

### d) N-(2-(Pyridin-3-yl)phenethyl)-2H-benzo[e][1,2,4]thiadiazine-3-carboxamide 1,1-dioxide (34)

A mixture of 2-(2-(pyridin-3-yl)phenyl)ethan-1-amine (A19) (0.043 g, 0.22 mmol), ethyl 2H-benzo[e][1,2,4]thiadiazine-3-carboxylate 1,1-dioxide (12) (0.050 g, 0.20 mmol) and EtOH (1.5 mL) was stirred in a sealed vessel at 110° C. for 1 hour and then at 120° C. for 2 h. The mixture was cooled to room temperature, the volatiles were removed in vacuo and the crude product purified by column chromatography (Biotage Isolera, 12 g SiO₂ cartridge, 0-100% EtOAc in petroleum benzine 40-60° C.) to give the product as a white solid (0.016 g, 20% yield): ¹H NMR (400 MHz, DMSO-d₆) δ 12.54 (s, 1H), 9.32-9.09 (m, 1H), 8.57 (dd, J=4.8, 1.6 Hz, 1H), 8.55-8.53 (m, 1H), 7.84 (dd, J=8.0, 1.4 Hz, 1H), 7.82-7.75 (m, 2H), 7.75-7.69 (m, 1H), 7.55-7.48 (m, 1H), 7.45-7.38 (m, 3H), 7.35-7.29 (m, 1H), 7.25-7.18 (m, 1H), 3.39-3.34 (m, 2H), 2.84 (t, J=7.4 Hz, 2H); LCMS-B rt 2.95 min; m/z 407.1 [M+H]⁺.

### Example 35: N-(2-Cyanophenethyl)-2H-benzo[e][1,2,4]thiadiazine-3-carboxamide 1,1-dioxide (35)

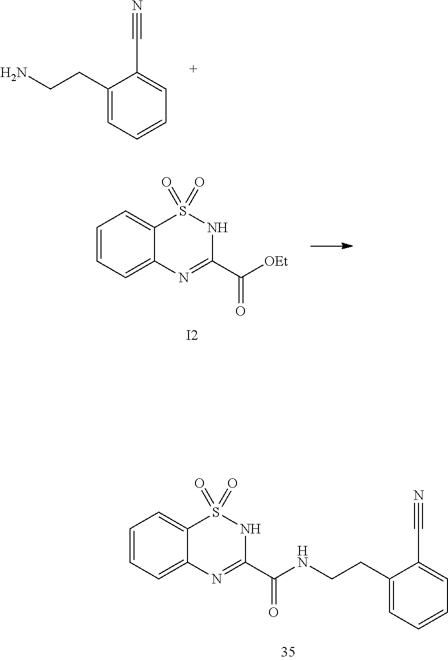 [SMILES: N#Cc1ccccc1CCN ; CCOC(=O)C1=Nc2ccccc2S(=O)(=O)N1 ; "I2" ; N#Cc1ccccc1CCNC(=O)C1=Nc2ccccc2S(=O)(=O)N1]

A mixture of ethyl 2H-benzo[e][1,2,4]thiadiazine-3-carboxylate 1,1-dioxide (12) (0.166 g, 0.655 mmol) and 2-(2-aminoethyl)benzonitrile (0.134 g, 0.917 mmol) in EtOH (1.5 mL) was heated in the microwave at 120° C. for 60 min. The mixture was returned to room temperature and the solvent removed in vacuo. The solid was taken up in DCM:MeOH (1:1 v/v) and loaded on to a Bond Elut SCX cartridge (10 g). The cartridge was eluted with DCM:MeOH (1:1 v/v, ˜100 mL) and the filtrate was concentrated in vacuo to give the product as a white solid (0.118 g, 51% yield): ¹H NMR (400 MHz, DMSO-d₆) δ 12.61 (s, 1H), 9.39 (t, J=6.0 Hz, 1H), 7.88-7.77 (m, 3H), 7.76-7.70 (m, 1H), 7.64 (td, J=7.9, 1.3 Hz, 1H), 7.53 (t, J=7.5 Hz, 1H), 7.48 (d, J=7.8 Hz, 1H), 7.43 (t, J=7.6 Hz, 1H), 3.59 (q, J=6.7 Hz, 2H), 3.10 (t, J=6.9 Hz, 2H); LCMS-A RT 4.15 min; m/z 355.2 [M+H]⁺.

### Example 36: 7-fluoro-N-(2-(oxazol-2-yl)-2-phenylethyl)-2H-benzo[e][1,2,4]thiadiazine-3-carboxamide 1,1-dioxide (36)

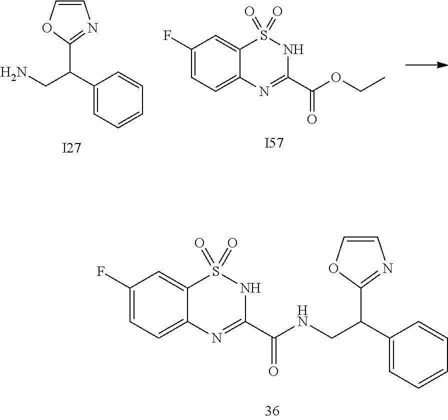 [SMILES: "I57" ; "I27" ; NCC(c1ccccc1)c1ncco1 ; CCOC(=O)C1=Nc2ccc(F)cc2S(=O)(=O)N1 ; O=C(NCC(c1ccccc1)c1ncco1)C1=Nc2ccc(F)cc2S(=O)(=O)N1]

2-(Oxazol-2-yl)-2-phenylethan-1-amine (I27) (0.026 g, 0.138 mmol) and ethyl 7-fluoro-2H-benzo[e][1,2,4]thiadiazine-3-carboxylate 1,1-dioxide (I57) (0.031 g, 80% purity, 0.092 mmol) were placed in a microwave vial. Dry EtOH (0.125 mL) was added and the reaction was subjected to microwave irradiation at 120° C. for 1 hour. The reaction was allowed to cool to room temperature and the sides of the tube were continuously scratched with a spatula for about 2 min. The precipitated solid was collected by filtration, washed with EtOH (2 mL) and dried under high-vacuum to give the product (0.020, 53% yield) as an off-white solid. ¹H NMR (400 MHz, d-DMSO) δ 9.28-9.17 (m, 1H), 8.04 (d, J=0.9 Hz, 1H), 7.82 (dd, J=9.8, 4.6 Hz, 2H), 7.72 (dd, J=7.6, 2.8 Hz, 1H), 7.63 (td, J=8.9, 2.9 Hz, 1H), 7.37-7.30 (m, 2H), 7.30-7.23 (m, 3H), 7.20 (d, J=0.9 Hz, 1H), 4.66 (t, J=7.5 Hz, 1H), 4.04-3.95 (m, 1H), 3.91-3.83 (m, 1H). LCMS-B: RT 3.22 min; m/z 415.0 [M+H]⁺.

### Example 37: N-(2,2-diphenylethyl)-7-(pyridin-3-yl)-2H-benzo[e][1,2,4]thiadiazine-3-carboxamide 1,1-dioxide (37)

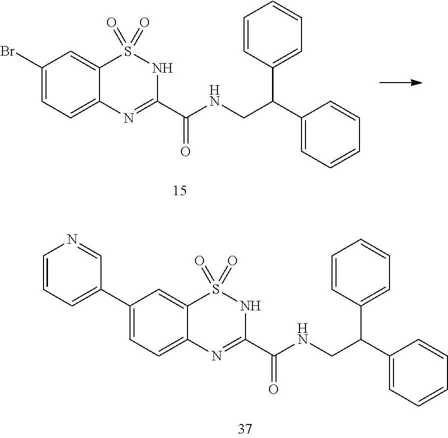 [SMILES: O=C(NCC(c1ccccc1)c1ccccc1)C1=Nc2ccc(Br)cc2S(=O)(=O)N1 ; O=C(NCC(c1ccccc1)c1ccccc1)C1=Nc2ccc(-c3cccnc3)cc2S(=O)(=O)N1]

7-bromo-N-(2,2-diphenylethyl)-2H-benzo[e][1,2,4]thiadiazine-3-carboxamide 1,1-dioxide (15) (50 mg, 0.10 mmol), pyridine 3-boronic acid (19 mg, 0.16 mmol), potassium carbonate (43 mg, 0.31 mmol) and PEPPSI-IPr (4 mg, 5 mol % yield) were loaded into a microwave tube and flushed with nitrogen. Absolute ethanol (1 mL) was added, the mixture degassed with a stream of nitrogen bubbles and heated in the microwave (80° C. for 30 min). The mixture was cooled to room temperature and then added to water (30 mL). The mixture was stirred and the pH adjusted to 3-4 with 30% w/v aq NaHSO₄. The precipitate was collected by centrifugation and dried azeotropically with ethanol. The mixture was slurried in 10% v/v MeOH/DCM (5 mL) and the solvent decanted. The remaining precipitate was purified by preparative TLC (100% ethyl acetate) to give the product (1 mg, 2% yield). LCMS-A: RT 5.60 min; m/z 481.1 [M−H]⁻.

### Example 38: N-(2,2-diphenylethyl)-7-ethynyl-2H-benzo[e][1,2,4]thiadiazine-3-carboxamide 1,1-dioxide (38)

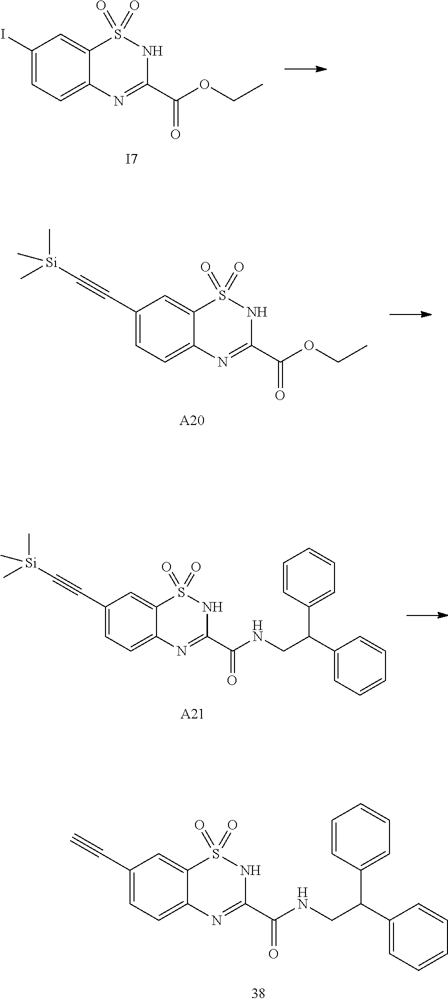

### a) Ethyl 7-((trimethylsilyl)ethynyl)-2H-benzo[e][1,2,4]thiadiazine-3-carboxylate 1,1-dioxide (A20)

Ethyl 7-iodo-2H-benzo[e][1,2,4]thiadiazine-3-carboxylate 1,1-dioxide (I7) (190 mg, 0.50 mmol), copper(I) iodide (5 mg, 5 mol % yield), bis(triphenylphosphine)palladium(II) dichloride (18 mg, 5 mol % yield), triethylamine (filtered through neutral alumina, 2 mL) and DMF (2 mL) were degassed with a stream of nitrogen bubbles. Trimethylsilylacetylene (0.214 mL, 1.5 mmol) was added and the mixture stirred at room temperature. After three days the mixture was poured into 0.5M aq HCl (60 mL) and extracted with DCM (3×30 mL). The pooled organic extracts were washed with brine (100 ml), dried over sodium sulfate and evaporated. Chromatography (12 g silica cartridge, 0-60% ethyl acetate/hexanes) gave the product as a pale yellow solid (101 mg, 58% yield). ¹H NMR (400 MHz, Chloroform-d) δ 9.48 (s, 1H), 8.07 (d, J=1.7 Hz, 1H), 7.66 (dd, J=8.5, 1.8 Hz, 1H), 7.15 (d, J=8.5 Hz, 1H), 4.51 (q, J=7.2 Hz, 2H), 1.46 (t, J=7.1 Hz, 3H), 0.26 (s, 9H). LCMS-B: 3.49 min; m/z 348.8 [M−H]⁻.

### b) N-(2,2-diphenylethyl)-7-((trimethylsilyl)ethynyl)-2H-benzo[e][1,2,4]thiadiazine-3-carboxamide 1,1-dioxide (A21)

Ethyl 7-((trimethylsilyl)ethynyl)-2H-benzo[e][1,2,4]thiadiazine-3-carboxylate 1,1-dioxide (A20) (25 mg, 0.071 mmol), 2,2-diphenylethan-1-amine (18 mg, 0.091 mmol) and absolute ethanol (1 mL) were heated in the microwave (100° for 1 h). The mixture was stood at room temperature for one hour and the resulting precipitate collected by filtration, washed with cold absolute ethanol (2×1 mL) and air dried to give the product as an off-white solid (8 mg, 22% yield). ¹H NMR (400 MHz, DMSO-d6) δ 9.24 (s, 1H), 7.84-7.78 (m, 1H), 7.75 (s, 2H), 7.35-7.25 (m, 9H), 7.25-7.15 (m, 2H), 4.48 (t, J=7.9 Hz, 1H), 3.92 (dd, J=7.8, 6.0 Hz, 2H), 0.24 (s, 9H). LCMS-A RT 6.76 min; m/z 500.1 [M−H]⁻.

### c) N-(2,2-diphenylethyl)-7-ethynyl-2H-benzo[e][1,2,4]thiadiazine-3-carboxamide 1,1-dioxide (38)

N-(2,2-Diphenylethyl)-7-((trimethylsilyl)ethynyl)-2H-benzo[e][1,2,4]thiadiazine-3-carboxamide 1,1-dioxide (A21) (7 mg, 0.012 mmol) was dissolved in 1:1 v/v MeOH:THF (1 mL) and a 1M aqueous solution of KOH (0.05 mL, 0.05 mmol) was added. After 30 min Dowex-50X8 H-form (200 mg) was added, the mixture filtered through a syringe filter and the resin washed with methanol (1 mL). The pooled filtrates were concentrated in vacuo, the residue rinsed with diethyl ether and dried in vacuo to give the product as a pale yellow solid (6 mg, quantitative yield). ¹H NMR (400 MHz, Acetone-d6) δ 8.60-8.53 (m, 1H), 7.90 (d, J=1.7 Hz, 1H), 7.83 (dd, J=8.6, 0.6 Hz, 1H), 7.79 (dd, J=8.6, 1.8 Hz, 1H), 7.39-7.27 (m, 8H), 7.23-7.17 (m, 2H), 4.56 (t, J=8.0 Hz, 1H), 4.15-4.07 (m, 2H), 3.88 (s, 1H), NH proton not observed. LCMS-B: RT 3.47 min; m/z 427.8 [M−H]⁻.

### Example 39: 7-bromo-N-(2-(4-fluorophenyl)-2-(pyridin-2-yl)ethyl)-2H-benzo[e][1,2,4]thiadiazine-3-carboxamide 1,1-dioxide (39)

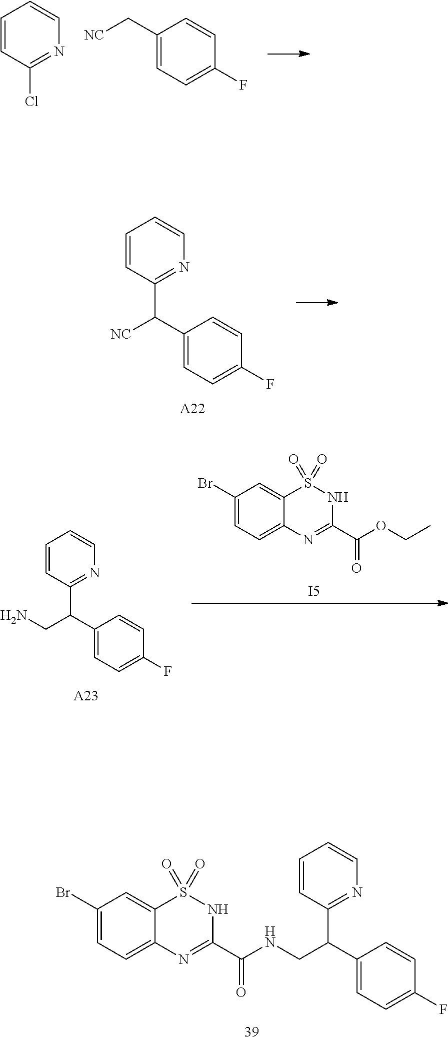

### a) 2-(4-fluorophenyl)-2-(pyridin-2-yl)acetonitrile (A22)

2-Chloropyridine (0.095 mL, 1.0 mmol) and 2-(4-fluorophenyl)acetonitrile (0.240 mL, 2.0 mmol) were dissolved in dry toluene (1 mL) and a 1.0M solution of NaHMDS in THF (2.0 mL, 2.0 mmol) was added. The mixture was stirred at room temperature overnight, filtered through a syringe filter and loaded onto a 12 g silica column. Chromatography (0-50% ethyl acetate/hexanes) gave the product as an oil (118 mg, 56% yield). ¹H NMR (400 MHz, Chloroform-d) δ 8.60 (ddd, J=4.9, 1.9, 0.9 Hz, 1H), 7.72 (td, J=7.7, 1.8 Hz, 1H), 7.46-7.36 (m, 3H), 7.29-7.22 (m, overlaps with CHCl₃), 7.11-7.01 (m, 2H), 5.29 (s, 1H). LCMS-A RT 4.02 min; m/z 213.1 [M+H]⁺.

### b) 2-(4-fluorophenyl)-2-(pyridin-2-yl)ethan-1-amine (A23)

2-(4-Fluorophenyl)-2-(pyridin-2-yl)acetonitrile (A22) (115 mg, 0.54 mmol) and cobalt(II) chloride (106 mg, 0.81 mmol) were dissolved in methanol (10 mL) and cooled to 0° C. under nitrogen. Sodium borohydride (103 mg, 2.71 mmol) was added in one portion under strong nitrogen flow. The mixture was stirred at room temperature under nitrogen for 45 min. The mixture was quenched with 3M aq HCl (2 mL) and concentrated in vacuo. Water (10 mL) and ethyl acetate (10 mL) were added, the pH of the aqueous phase was adjusted to 13 with 20% w/v aq NaOH and the mixture filtered through Celite®. The separated aqueous phase was extracted with further ethyl acetate (2×10 mL), the pooled ethyl acetate phases dried over sodium sulfate and evaporated to give the product as a pale yellow syrup (26 mg, 22% yield). LCMS-A RT: 1.58 min; m/z (positive ion) 217.1 [M+H]

### c) 7-bromo-N-(2-(4-fluorophenyl)-2-(pyridin-2-yl)ethyl)-2H-benzo[e][1,2,4]thiadiazine-3-carboxamide 1,1-dioxide (39)

Ethyl 7-bromo-2H-benzo[e][1,2,4]thiadiazine-3-carboxylate 1,1-dioxide (I5) (33 mg, 0.10 mmol), 2-(4-fluorophenyl)-2-(pyridin-2-yl)ethan-1-amine (A23) (26 mg, 0.12 mmol) and ethanol (1 mL) were heated in the microwave at 100° C. for 30 min. The mixture was cooled to room temperature and filtered. The filtrate was purified by preparative TLC (60% ethyl acetate/hexanes) followed by recrystallization from acetonitrile to give the product as a white solid (10 mg, 19% yield). ¹H NMR (400 MHz, DMSO-d6) δ 9.23 (t, J=6.0 Hz, 1H), 8.56 (ddd, J=4.8, 1.8, 0.9 Hz, 1H), 7.99 (d, J=2.2 Hz, 1H), 7.92 (dd, J=8.9, 2.2 Hz, 1H), 7.77-7.68 (m, 2H), 7.41-7.35 (m, 2H), 7.32 (d, J=7.9 Hz, 1H), 7.25 (ddd, J=7.5, 4.8, 1.1 Hz, 1H), 7.15-7.05 (m, 2H), 4.60 (t, J=7.5 Hz, 1H), 4.08-3.91 (m, 2H). LCMS-B RT 3.33 min; m/z 502.7 [M+H]⁺.

### Example 40: N-(2-(1-methyl-1H-pyrazol-4-yl)phenethyl)-2H-benzo[e][1,2,4]thiadiazine-3-carboxamide 1,1-dioxide (40)

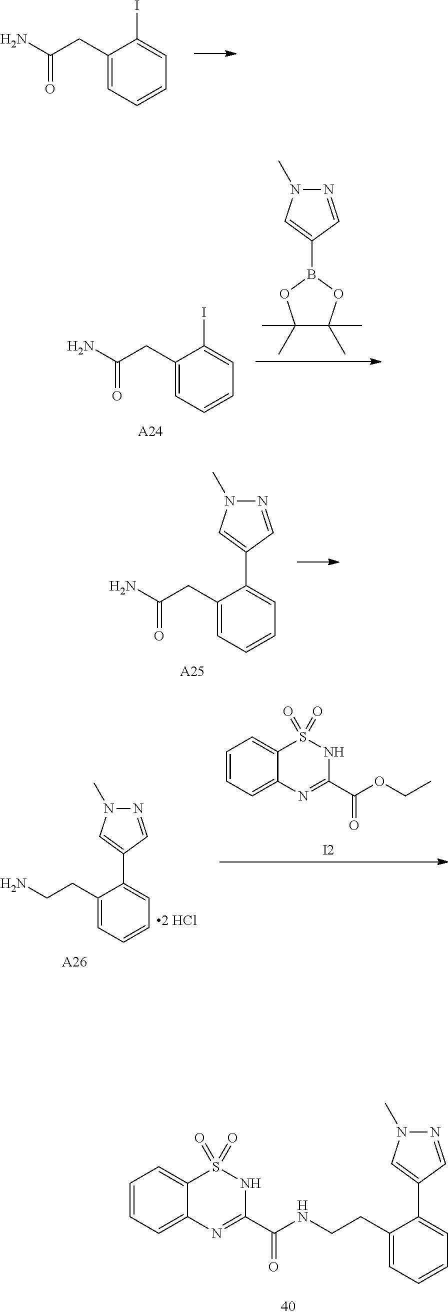

### a) 2-(2-Iodophenyl)acetamide (A24)

2-Iodophenylacetic acid (2.62 g, 10.0 mmol), DCM (50 mL), oxalyl chloride (1.03 mL, 12.0 mmol) and DMF (0.05 mL) were stirred at room temperature. After one hour the mixture was concentrated in vacuo. The residue was dissolved in THF (50 mL) and a concentrated solution of aqueous ammonia (50 mL) added. The mixture was stirred for thirty min and concentrated in vacuo. The residue was slurried in water (100 mL), filtered, the collected solid washed with water (2×50 mL) and air dried to give the product as a tan solid (2.33 g, 89% yield). ¹H NMR (400 MHz, Chloroform-d) δ 7.90-7.86 (m, 1H), 7.40-7.33 (m, 2H), 7.03-6.96 (m, 1H), 5.42 (brs, 2H), 3.75 (s, 2H). LCMS-A RT 4.88 min; m/z 262.0 [M+H]⁺.

### b) 2-(2-(1-Methyl-1H-pyrazol-4-yl)phenyl)acetamide (A25)

2-(2-Iodophenyl)acetamide (A24) (261 mg, 1.00 mmol), 1-methyl-4-(4,4,5,5-tetramethyl-1,3,2-dioxaborolan-2-yl)-1H-pyrazole (312 mg, 1.50 mmol), cesium carbonate (977 mg, 3.00 mmol), Pd(PPh₃)₂Cl₂ (35 mg, 5 mol % yield) and 1,4-dioxane (5 mL) were loaded into a microwave tube. The mixture was degassed with a stream of nitrogen bubbles and heated in the microwave (120° C. for 5 min). The mixture was cooled to room temperature, diluted with ethyl acetate (20 mL) and filtered through celite. The filtrate was concentrated in vacuo and separated by chromatography (12 g silica cartridge, 0-100% ethyl acetate/hexanes then 0-100% methanol/ethyl acetate) gave the product as a yellow oil (12 mg, 6% yield). ¹H NMR (400 MHz, Methanol-d4) δ 7.75-7.72 (m, 1H), 7.58 (d, J=0.8 Hz, 1H), 7.36-7.25 (m, 4H), 3.94 (s, 3H), 3.61 (s, 2H). LCMS-A RT 4.51 min; m/z 216.2 [M+H]⁺.

### c) 2-(2-(1-Methyl-1H-pyrazol-4-yl)phenyl)ethan-1-amine bis(hydrochloride) (A26)

2-(2-(1-Methyl-1H-pyrazol-4-yl)phenyl)acetamide (A25) (12 mg, 0.056 mmol) and 1.0M borane in THF (0.50 mL, 0.50 mmol) were heated to 80° C. overnight. A 3M aq HCl solution (1 mL) was added and the mixture returned to 80° C. for thirty min then concentrated in vacuo. The residue was loaded onto a 0.5 g SCX cartridge, washed with methanol (10 mL) and eluted with 7M ammonia in methanol (10 mL). The basic eluate was concentrated in vacuo, and the residue dissolved and concentrated twice from methanol. The residue was dissolved in 1,4-dioxane (0.5 mL) and 4.0M HCl/1,4-dioxane (0.5 mL) added. The mixture was concentrated in vacuo, the solid residue slurried in ether (2 mL), the ether decanted and the solid dried in vacuo to give the product as a white solid (18 mg). The material was carried forward without further purification. LCMS-B: RT 2.65 min; m/z 202.0 [M+H]⁺ for the free base.

### d) N-(2-(1-Methyl-1H-pyrazol-4-yl)phenethyl)-2H-benzo[e][1,2,4]thiadiazine-3-carboxamide 1,1-dioxide (40)

Ethyl 2H-benzo[e][1,2,4]thiadiazine-3-carboxylate 1,1-dioxide (I2) (12 mg, 0.047 mmol), 2-(2-(1-methyl-1H-pyrazol-4-yl)phenyl)ethan-1-amine bis(hydrochloride) (A26) (0.056 mmol at 100% conversion), triethylamine (0.016 mL, 0.11 mmol) and ethanol (1 mL) were heated in the microwave (100° C. for 1 hour then 120° C. for 30 min). The mixture was separated by preparative TLC (100% ethyl acetate) to give the product as a white solid. LCMS-B RT 3.22 min; m/z 409.9 [M+H]⁺; m/z 407.9 [M−H]⁻.

### Examples 41-43

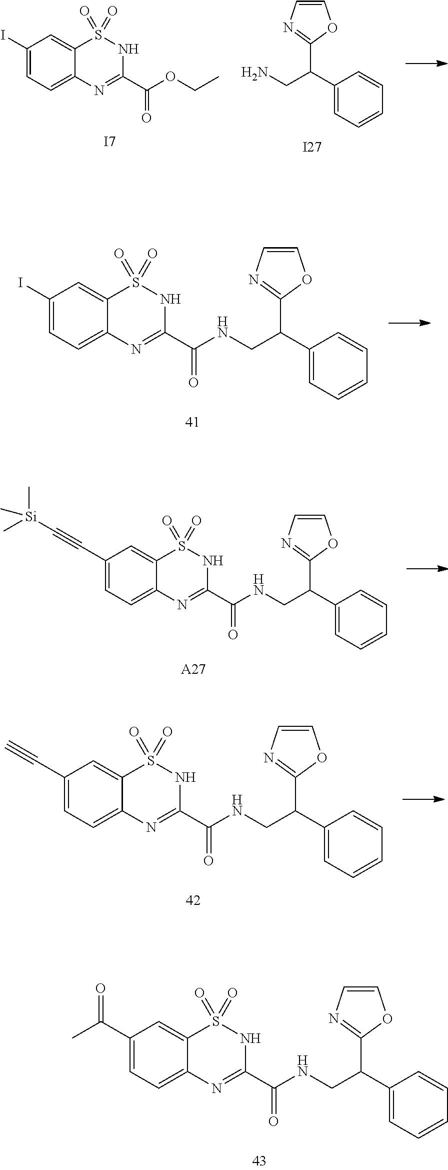

### a) 7-iodo-N-(2-(oxazol-2-yl)-2-phenylethyl)-2H-benzo[e][1,2,4]thiadiazine-3-carboxamide 1,1-dioxide (41)

Ethyl 7-iodo-2H-benzo[e][1,2,4]thiadiazine-3-carboxylate 1,1-dioxide (I7) (100 mg, 0.26 mmol), 2-(oxazol-2-yl)-2-phenylethan-1-amine (I27) (59 mg, 0.32 mmol) and ethanol (1 mL) were heated in the microwave (100° C. for 1h). The mixture was cooled to room temperature, the precipitate filtered and the collected solids washed with cold ethanol (3×1 mL) and air dried to give the product as a pink solid (67 mg). Further material (9 mg) was recovered by concentration of the combined filtrates and purification by chromatography (4 g silica cartridge, 0-5% methanol/DCM). Total product: 74 mg, 54% yield. ¹H NMR (400 MHz, d-DMSO) δ 12.72 (br s, 1H), 9.32-9.20 (m, 1H), 8.12-7.99 (m, 3H), 7.56 (d, J=8.8 Hz, 1H), 7.36-7.24 (m, 5H), 7.20 (d, J=0.9 Hz, 1H), 4.66 (t, J=7.5 Hz, 1H), 4.05-3.94 (m, 1H), 3.91-3.81 (m, 1H). LCMS-B: rt 3.277 min; m/z 523.0 [M+H]⁺.

### b) N-(2-(oxazol-2-yl)-2-phenylethyl)-7-((trimethylsilyl)ethynyl)-2H-benzo[e][1,2,4]thiadiazine-3-carboxamide 1,1-dioxide (A27)

7-iodo-N-(2-(oxazol-2-yl)-2-phenylethyl)-2H-benzo[e][1,2,4]thiadiazine-3-carboxamide 1,1-dioxide (41) (72 mg, 0.14 mmol) was dissolved in NEt₃ (0.5 mL) and DMF (0.5 mL), CuI (1 mg, 5 mol % yield) and Pd(PPh₃)₂Cl₂ (5 mg, 5 mol % yield) were added and the mixture degassed with a stream of nitrogen bubbles. TMS-acetylene (0.057 mL, 0.41 mmol) was added and the mixture stirred overnight. The mixture was added to water (20 mL) and the pH adjusted to 5 with 3M HCl. The mixture was extracted with ethyl acetate (3×20 mL), the pooled ethyl acetate phases were washed with water (20 mL), brine (20 mL), dried over sodium sulfate and concentrated in vacuo. Chromatography (4 g silica cartridge, 0-80% ethyl acetate/hexanes) gave the product as a pale yellow solid (27 mg, 40% yield). ¹H NMR (400 MHz, Chloroform-d) δ 9.86 (s, 1H), 8.38 (t, J=6.5 Hz, 1H), 8.06 (d, J=1.7 Hz, 1H), 7.65-7.59 (m, 2H), 7.38-7.30 (m, 3H), 7.23-7.13 (m, 4H), 4.39 (t, J=7.0 Hz, 1H), 4.08 (t, J=6.7 Hz, 2H), 0.26 (s, 9H). LCMS-B RT 4.69 min; m/z 490.9 [M−H]⁻.

### c) 7-ethynyl-N-(2-(oxazol-2-yl)-2-phenylethyl)-2H-benzo[e][1,2,4]thiadiazine-3-carboxamide 1,1-dioxide (42)

N-(2-(oxazol-2-yl)-2-phenylethyl)-7-((trimethylsilyl)ethynyl)-2H-benzo[e][1,2,4]thiadiazine-3-carboxamide 1,1-dioxide (A27) (26 mg, 0.053 mmol) was dissolved in 1:1 v/v THF: MeOH (4 mL) and 1.0M aq KOH (0.185 mL, 0.19 mmol) was added. The mixture was stirred for 45 min then Dowex 50X8 Hi-form (0.8 g) added. The mixture was filtered and the resin washed with methanol (5 mL). The pooled filtrates were concentrated in vacuo, the residue dried azeotropically by evaporation from ethanol (2×2 mL), rinsed with ether and dried in vacuo to give the product as a tan solid (17 mg, 77% yield). ¹H NMR (400 MHz, DMSO-d6) δ 9.30 (t, J=5.9 Hz, 1H), 8.04 (d, J=0.9 Hz, 1H), 7.90-7.87 (m, 1H), 7.82-7.77 (m, 2H), 7.37-7.24 (m, 5H), 7.20 (d, J=0.9 Hz, 1H), 4.67 (t, J=7.6 Hz, 1H), 4.44 (s, 1H), 4.00 (ddd, J=13.2, 7.6, 5.7 Hz, 1H), 3.92-3.83 (m, 1H). LCMS-A RT 5.63 min; m/z 421.1 [M+H]⁺.

### d) 7-acetyl-N-(2-(oxazol-2-yl)-2-phenylethyl)-2H-benzo[e][1,2,4]thiadiazine-3-carboxamide 1,1-dioxide (43)

Chloro[1,3-bis(2,6-diisopropylphenyl)imidazol-2-ylidene]gold(I) (1.5 mg, 20 mol % yield), silver hexafluoroantimonate (0.8 mg, 20 mol % yield) and methanol (1 mL) were stirred at room temperature for two min. 7-ethynyl-N-(2-(oxazol-2-yl)-2-phenylethyl)-2H-benzo[e][1,2,4]thiadiazine-3-carboxamide 1,1-dioxide (42) (5.0 mg, 0.012 mmol) and milliQ water (0.5 mL) were added and the mixture stirred at 65° C. overnight. The mixture was cooled to room temperature, diluted to 10 mL with methanol and mixed vigorously. Thiourea functionalised silica (SiliaMet thiourea, 1.1 mmol/g, 12 mg) was added and the mixture stirred vigorously at room temperature for one hour. The mixture was filtered through a syringe filter and the filtrate concentrated in vacuo. The residue was suspended in ethanol (20 mL) and again concentrated in vacuo. The residue was extracted with hot methanol (1 mL), the solvent was decanted and concentrated in vacuo. The residue was dissolved in methanol (1 mL) and treated with thiol functionalised silica (SiliaMet thiol, 1.4 mmol/g, 10 mg) for thirty min. The mixture was filtered through a syringe filter and the filtrate concentrated in vacuo to give the product as a yellow solid (3.2 mg, 61% yield). LCMS-B: RT 3.17 min; m/z 438.8 [M+H]⁺; m/z 436.8 [M−H]⁻.

### Example 44: N-(3-oxo-2-phenyl-3-(pyrrolidin-1-yl)propyl)-2H-benzo[e][1,2,4]thiadiazine-3-carboxamide 1,1-dioxide (44)

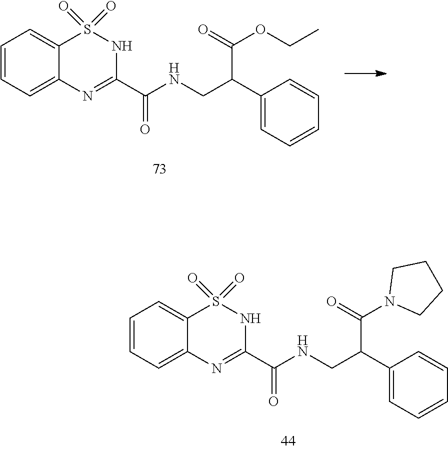 [SMILES: CCOC(=O)C(CNC(=O)C1=Nc2ccccc2S(=O)(=O)N1)c1ccccc1 ; O=C(NCC(C(=O)N1CCCC1)c1ccccc1)C1=Nc2ccccc2S(=O)(=O)N1]

A suspension of ethyl 3-(1,1-dioxido-2H-benzo[e][1,2,4]thiadiazine-3-carboxamido)-2-phenylpropanoate (73) (0.025 g, 0.062 mmol) and pyrrolidine (0.010 mL, 0.125 mmol) in EtOH (0.1 mL) were irradiated in a microwave reactor at 100° C. for 30 min. The mixture was further treated with NEt₃ (0.017 mL, 0.125 mmol) and pyrrolidine (0.04 mL), then irradiated at 150° C. for 1 h. The crude material was loaded directly onto a column and purified by silica gel chromatography (Isolera Biotage 4 g, 0-100% EtOAc in petroleum benzine 40-60° C., then 0-40% EtOAc in MeOH). The material was further purified by RP-HPLC (Grace Alltima, C8, 5 micron column, 250 mm×22 mm ID, 30-100% CH₃CN in water, 0.1% TFA over 20 min) to give the product (0.003 g, 11% yield) as a white solid. LCMS-B: RT 3.465 min; m/z 427.2 [M+H]⁺.

### Example 45: N-(3-(methylamino)-3-oxo-2-phenylpropyl)-2H-benzo[e][1,2,4]thiadiazine-3-carboxamide 1,1-dioxide (45)

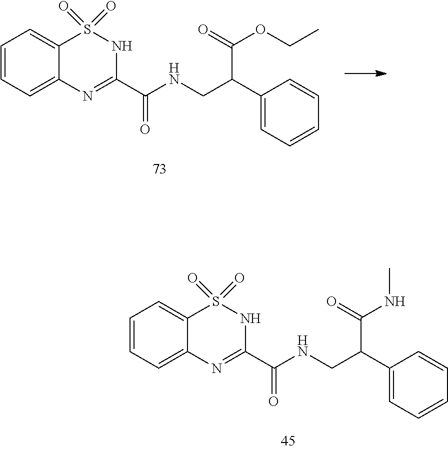 [SMILES: CCOC(=O)C(CNC(=O)C1=Nc2ccccc2S(=O)(=O)N1)c1ccccc1 ; CNC(=O)C(CNC(=O)C1=Nc2ccccc2S(=O)(=O)N1)c1ccccc1]

A suspension of ethyl 3-(1,1-dioxido-2H-benzo[e][1,2,4]thiadiazine-3-carboxamido)-2-phenylpropanoate (73) (0.025 g, 0.062 mmol), NEt₃ (0.017 mL, 0.125 mmol) and methylamine (0.016 mL, 0.125 mmol) in EtOH (0.1 mL) were irradiated in a microwave reactor at 150° C. for 30 min. The mixture was treated with additional equivalents of methylamine (0.016 mL, 0.125 mmol) and irradiated at 150° C. for a further 2 h. The material was loaded directly onto a column and purified by RP-HPLC (Grace Alltima, C8, 5 micron column, 250 mm×22 mm ID, 30-100% CH₃CN in water, 0.1% TFA over 20 min) to give the product (0.006 g, 25% yield) as a white solid. ¹H NMR (400 MHz, MeOD): δ 7.89 (dd, J=8.0, 1.1 Hz, 1H), 7.71 (ddd, J=8.6, 7.3, 1.4 Hz, 1H), 7.60 (dd, J=8.4, 0.7 Hz, 1H), 7.53 (ddd, J=8.3, 7.3, 1.1 Hz, 1H), 7.40-7.24 (m, 5H), 3.95-3.85 (m, 2H), 3.73 (td, J=10.7, 9.3 Hz, 1H), 2.70 (s, 3H). LCMS-B RT 3.366 min; m/z 387.2 [M+H]⁺.

### Example 46: N-(2-(oxazol-2-yl)-2-phenylethyl)-2H-benzo[e][1,2,4]thiadiazine-3-carboxamide 1,1-dioxide (46)

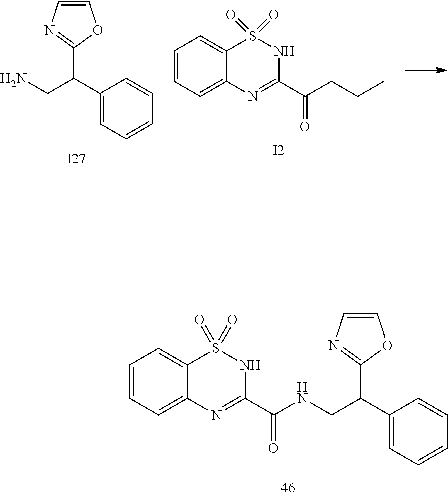

To a suspension of the ethyl 2H-benzo[e][1,2,4]thiadiazine-3-carboxylate 1,1-dioxide (I2) (0.063 g, 0.246 mmol) in EtOH (0.125 mL) was added 2-(oxazol-2-yl)-2-phenylethan-1-amine (I27) (0.051 g, 0.271 mmol). The mixture was subjected to microwave irradiation at 100° C. for 30 min. The reaction was cooled and the precipitate filtered. The solid was washed with EtOH (3 mL) and dried under vacuum to give the product (0.072 g, 74% yield) as a white solid. ¹H NMR (400 MHz, DMSO-d₆): δ 12.62 (brs, 1H), 9.28 (t, J=5.9 Hz, 1H), 8.05 (d, J=0.8 Hz, 1H), 7.84 (dd, J=8.0, 1.2 Hz, 1H), 7.79 (d, J=7.9 Hz, 1H), 7.75-7.69 (m, 1H), 7.55-7.49 (m, 1H), 7.36-7.30 (m, 2H), 7.30-7.24 (m, 3H), 7.20 (d, J=0.8 Hz, 1H), 4.67 (t, J=7.6 Hz, 1H), 4.00 (ddd, J=13.2, 7.5, 5.7 Hz, 1H), 3.92-3.84 (m, 1H). LCMS-B: rt 3.495 min, m/z 397.2 [M+H]⁺.

### Example 47: N-(2-(1,3,4-oxadiazol-2-yl)-2-phenylethyl)-2H-benzo[e][1,2,4]thiadiazine-3-carboxamide 1,1-dioxide (47)

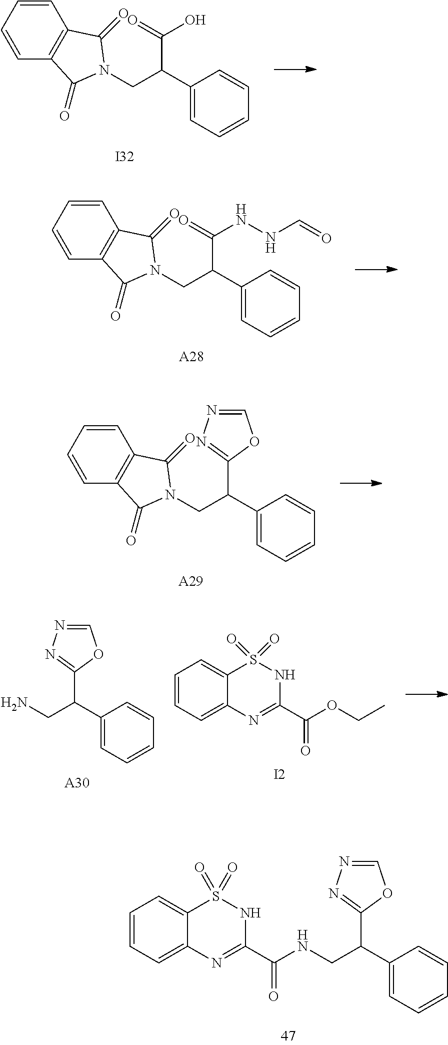

### a) 3-(1,3-dioxoisoindolin-2-yl)-N′-formyl-2-phenylpropanehydrazide (A28)

To a solution of 3-(1,3-dioxoisoindolin-2-yl)-2-phenylpropanoic acid (I32) (0.250 g, 0.847 mmol), EDCI (0.194 g, 1.016 mmol) and formic hydrazine (0.051 g, 0.847 mmol) in DCM (10 mL) was added DMAP (0.124 g, 1.016 mmol). This was allowed to stir at r.t. for 17 h, upon which time the mixture was treated with 1M HCl (10 mL). The layers were separated and the organic portion concentrated in vacuo to give the product (0.464 g, >100% yield) as a white solid. The material was carried forward without further purification. LCMS:B: rt. 3.346 min, m/z 336.1 [M−H]⁻.

### b) 2-(2-(1,3,4-oxadiazol-2-yl)-2-phenylethyl)isoindoline-1,3-dione (A29)

To a suspension of Burgess reagent (0.775 g, 3.253 mmol) in THF (4 mL) was added 3-(1,3-dioxoisoindolin-2-yl)-N′-formyl-2-phenylpropanehydrazide (28) (0.439 g, 1.301 mmol). This was irradiated in a microwave reactor at 140° C. for 15 min. Upon cooling, the mixture was loaded directly onto silica for purification. The crude material was purified by silica gel chromatography (Isolera Biotage 24 g, 0-100% EtOAc in petroleum benzine 40-60° C., then 0-40% MeOH in EtOAc). Product-containing fractions were combined and concentrated in vacuo to give the product (0.095 g, 23% yield) as a white solid. LCMS-B: rt. 3.558 min, m/z 320.2 [M+H]⁺.

### c) 2-(1,3,4-oxadiazol-2-yl)-2-phenylethan-1-amine (A30)

To a suspension of 2-(2-(1,3,4-oxadiazol-2-yl)-2-phenylethyl)isoindoline-1,3-dione (A29) (0.045 g, 0.141 mmol) in EtOH (2 mL) was added hydrazine hydrate (50-60%, 0.026 mL, 0.423-0.508 mmol). The solution was heated to 80° C. for 3 h, upon which time it was cooled and the precipitate filtered. The precipitate was washed with a portion of cold EtOH (5 mL), and the combined EtOH fractions were pooled and concentrated in vacuo to give the product (0.030 g, >100% yield) as a yellow semi-solid. The material was carried forward without further purification. LCMS-B: rt. 3.411; no product ion detectable.

### d) N-(2-(1,3,4-oxadiazol-2-yl)-2-phenylethyl)-2H-benzo[e][1,2,4]thiadiazine-3-carboxamide 1,1-dioxide (47)

To a suspension of 2-(1,3,4-oxadiazol-2-yl)-2-phenylethan-1-amine (A30) (0.030 g, 0.111 mmol) in EtOH (0.5 mL) was added ethyl 2H-benzo[e][1,2,4]thiadiazine-3-carboxylate 1,1-dioxide (12) (0.020 g, 0.079 mmol). This was irradiated in a microwave reactor at 100° C. for 30 min. The solution was cooled and the EtOH evaporated. The residue was partitioned between EtOAc (3 mL) and 1M HCl (3 mL). The organic layer was separated and washed with a further portion of 1M HCl (3 mL), brine (3 mL), dried (Na₂SO₄) and concentrated in vacuo. The material was purified by RP-HPLC (Grace Alltima, C8, 5 micron column, 250 mm×22 mm ID, 30-100% CH₃CN in water, 0.1% TFA over 20 min) to give the product (0.003 g, 10% yield) as a white solid. LCMS-B: rt. 3.420 min, m/z 398.1 [M+H]⁺.

### Example 48: N-(2-(5-methyl-1,3,4-oxadiazol-2-yl)-2-phenylethyl)-2H-benzo[e][1,2,4]thiadiazine-3-carboxamide 1,1-dioxide (48)

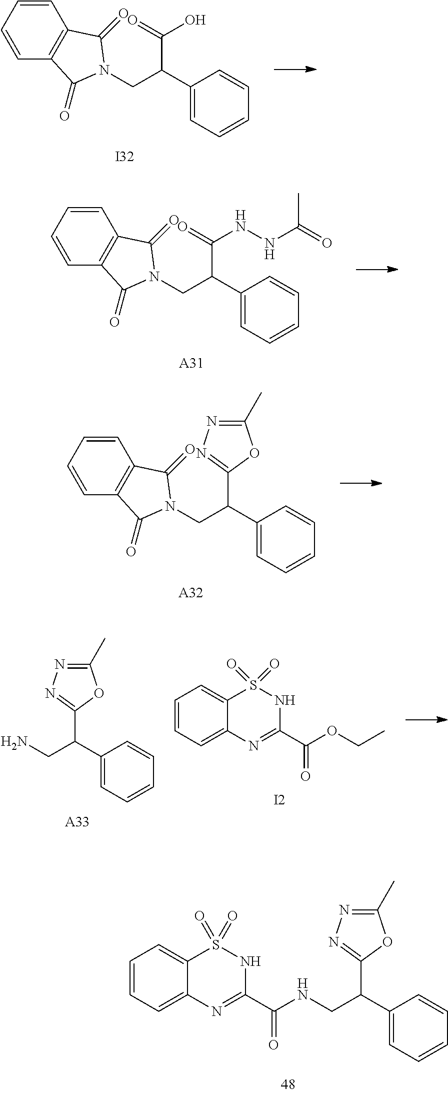

### a) N′-acetyl-3-(1,3-dioxoisoindolin-2-yl)-2-phenylpropanehydrazide (A31)

To a solution of 3-(1,3-dioxoisoindolin-2-yl)-2-phenylpropanoic acid (I32) (0.500 g, 1.693 mmol), EDCI (0.387 g, 2.032 mmol) and formic hydrazine (0.125 g, 1.693 mmol) in DCM (20 mL) was added DMAP (0.248 g, 2.032 mmol). This was allowed to stir at r.t. for 17 h, upon which time the mixture was treated with 1M HCl (20 mL). The layers were separated and the organic portion concentrated in vacuo to give the product (0.680 g, >100% yield) as a white solid. The material was carried forward without further purification. ¹H NMR (400 MHz, DMSO-d6): δ 10.15 (d, J=2.0 Hz, 1H), 9.77 (d, J=1.9 Hz, 1H), 7.81-7.78 (m, 4H), 7.33-7.28 (m, 2H), 7.27-7.16 (m, 3H), 4.25 (dd, J=8.9, 7.1 Hz, 1H), 4.08 (dd, J=13.7, 9.0 Hz, 1H), 3.96 (dd, J=13.7, 7.2 Hz, 1H), 1.79 (s, 3H). LCMS-B: rt. 3.324, m/z 350.1 [M−H]⁻.

### b) 2-(2-(5-methyl-1,3,4-oxadiazol-2-yl)-2-phenylethyl)isoindoline-1,3-dione (A32)

To a suspension of Burgess reagent (1.153 g, 4.838 mmol) in THF (7 mL) was added N′-acetyl-3-(1,3-dioxoisoindolin-2-yl)-2-phenylpropanehydrazide (A31) (0.680 g, 1.935 mmol). This was irradiated in a microwave reactor at 140° C. for 15 min. Upon cooling, the crude material was loaded onto silica gel and purified by silica gel chromatography (Isolera Biotage, 40 g SiO₂ Cartridge, 0-100% EtOAc in petroleum benzine 40-60° C.). The fractions containing the desired product were collected and concentrated in vacuo to yield the product (0.289 g, 45% yield) as a white solid. ¹H NMR (400 MHz, DMSO-d6): δ 7.87-7.79 (m, 4H), 7.35-7.31 (m, 4H), 7.31-7.26 (m, 1H), 4.76 (t, J=8.0 Hz, 1H), 4.28 (dd, J=13.9, 7.7 Hz, 1H), 4.21 (dd, J=13.9, 8.3 Hz, 1H), 4.03 (q, J=7.1 Hz, 1H), 2.43 (s, 3H). LCMS-B: rt. 3.588, m/z 334.2 [M+H]⁺.

### c) 2-(5-methyl-1,3,4-oxadiazol-2-yl)-2-phenylethan-1-amine (A33)

To a suspension of 2-(2-(5-methyl-1,3,4-oxadiazol-2-yl)-2-phenylethyl)isoindoline-1,3-dione (A32) (0.285 g, 0.855 mmol) in EtOH (12 mL) was added hydrazine hydrate (50-60%, 0.160 mL, 2.57-3.08 mmol). The solution was heated to 80° C. for 3 h, upon which time it was cooled and the precipitate filtered. The precipitate was washed with a portion of cold

EtOH (5 mL), and the combined EtOH fractions were pooled and concentrated in vacuo to give the product (0.174 g, >100% yield) as a yellow oil. The material was carried forward without further purification. LCMS-B: rt. 3.121; no product ion detectable.

### d) N-(2-(5-methyl-1,3,4-oxadiazol-2-yl)-2-phenylethyl)-2H-benzo[e][1,2,4]thiadiazine-3-carboxamide 1,1-dioxide (48)

To a suspension of 2-(5-methyl-1,3,4-oxadiazol-2-yl)-2-phenylethan-1-amine (A33) (0.100 g, 0.492 mmol) in EtOH (0.25 mL) was added ethyl 2H-benzo[e][1,2,4]thiadiazine-3-carboxylate 1,1-dioxide (12) (0.114 g, 0.447 mmol). This was irradiated in a microwave reactor at 100° C. for 30 min. The solution was cooled and the precipitate filtered. The resulting solid was washed with further portions of EtOH (3×3 mL) and dried to reveal the desired product (0.131 g, 71% yield) as a white solid. ¹H NMR (400 MHz, DMSO-d6): δ 12.60 (brs, 1H), 9.30 (brs, 1H), 7.86-7.80 (m, 1H), 7.73 (dt, J=14.4, 7.7 Hz, 2H), 7.50 (t, J=7.5 Hz, 1H), 7.42-7.29 (m, 5H), 4.72 (t, J=7.5 Hz, 1H), 4.00 (ddd, J=13.6, 8.0, 5.8 Hz, 1H), 3.87 (dt, J=13.4, 6.7 Hz, 1H), 2.44 (s, 3H). LCMS-B: rt. 3.408 min, m/z 412.2 [M+H]⁺.

### Example 49: Ethyl (2-(1,1-dioxido-2H-benzo[e][1,2,4]thiadiazine-3-carboxamido)-1-phenylethyl)carbamate (49)

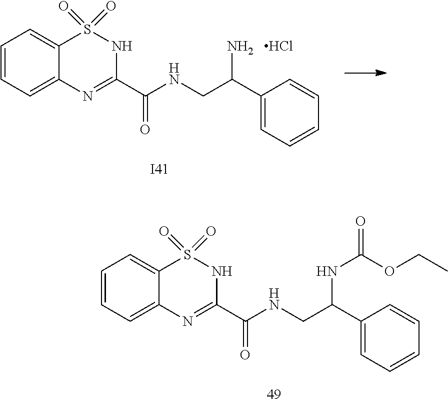

To a suspension of N-(2-amino-2-phenylethyl)-2H-benzo[e][1,2,4]thiadiazine-3-carboxamide 1,1-dioxide hydrochloride (I41) (0.025 g, 0.066 mmol) in DCM (0.5 mL) was added NEt₃ (0.019 mL, 0.135 mmol), followed 10 min later by ethyl chloroformate (0.007 mL, 0.069 mmol) dropwise. This was allowed to stir at r.t. for 17 h upon which time the reaction was diluted with DCM (1 mL), washed with 1M HCl (1 mL), saturated Na₂CO₃ (1 mL), brine (1 mL) then dried (Na₂SO₄) and concentrated in vacuo to reveal the product (0.022 g, 80% yield) as a white solid. LCMS-B: r.t. 3.474 min; m/z 417.2 [M+H]⁺.

### Example 50: Isopropyl (2-(1,1-dioxido-2H-benzo[e][1,2,4]thiadiazine-3-carboxamido)-1-phenylethyl) carbamate (50)

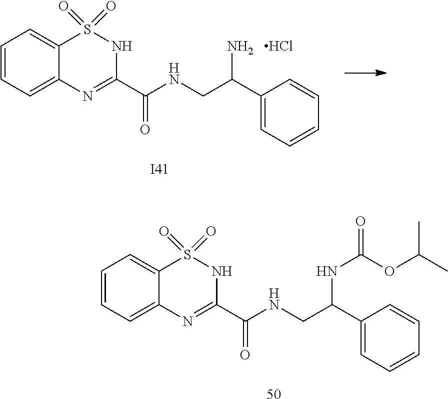

To a suspension of N-(2-amino-2-phenylethyl)-2H-benzo[e][1,2,4]thiadiazine-3-carboxamide 1,1-dioxide hydrochloride (I41) (0.020 g, 0.053 mmol) in DCM (0.5 mL) was added NEt₃ (0.015 mL, 0.111 mmol), followed 10 min later by a 1M solution of iso-propyl chloroformate (0.061 mL, 0.064) dropwise. This was allowed to stir at r.t. for 2 h upon which the reaction was diluted with DCM (1 mL) and washed with 1M HCl (1 mL), saturated Na₂CO₃ (1 mL), brine (1 mL) then dried (Na₂SO₄) and concentrated in vacuo. The crude material was purified by RP-HPLC (Grace Alltima, C8, 5 micron column, 250 mm×22 mm ID, 30-100% CH₃CN in water, 0.1% TFA over 30 min) to give the product (0.002 g, 7% yield) as a white solid. LCMS-B: r.t. 3.519 min; m/z 429.2 [M−H]⁻.

### Example 51: 2-(1,1-dioxido-2H-benzo[e][1,2,4]thiadiazine-3-carboxamido)-1-phenylethyl azetidine-1-carboxylate (51)

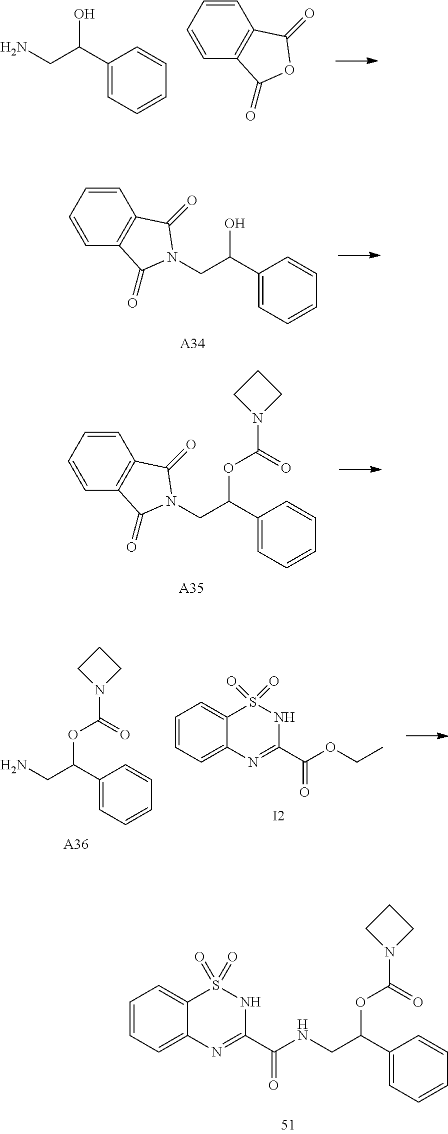

### a) 2-(2-hydroxy-2-phenylethyl)isoindoline-1,3-dione (A34)

Phthalic anhydride (2.159 g, 14.579 mmol) and 2-amino-1-phenylethan-1-ol (2.000 g, 14.579 mmol) were combined in a microwave vessel and irradiated at 150° C. for 15 min. The resulting residue was dried under vacuum to reveal the desired product (3.600 g, 92% yield) as a white solid. ¹H NMR (400 MHz, DMSO-d6): δ 7.89-7.81 (m, 4H), 7.41-7.32 (m, 4H), 7.30-7.23 (m, 1H), 5.66 (brs, 1H), 4.93 (dd, J=8.8, 4.8 Hz, 1H), 3.77 (dd, J=13.6, 8.8 Hz, 1H), 3.64 (dd, J=13.6, 4.8 Hz, 1H). LCMS-B: rt 3.567 min; m/z 266.1 [M−H]⁻.

### b) 2-(1,3-dioxoisoindolin-2-yl)-1-phenylethyl azetidine-1-carboxylate (A35)

To a solution of 2-(2-hydroxy-2-phenylethyl)isoindoline-1,3-dione (A34) (0.400 g, 1.497 mmol) in dry toluene (5 mL), under an atmosphere of N₂, was added CDI (0.291 g, 1.796 mmol). The mixture was allowed to stir at r.t. for 3 h, upon which dry THF (2 mL) was added. The solution was stirred for a further hour, upon which azetidine.HCl (0.280 g, 2.993 mmol) was added. The mixture was left to stir overnight. EtOAc was added (10 mL) and the mixture was then washed with water (10 mL), brine (10 mL), dried (Na₂SO₄), filtered and concentrated in vacuo. This crude material was purified by column chromatography (Isolera Biotage, 40 g SiO₂ Cartridge, 0-100% EtOAc in petroleum benzine 40-60° C.), with the fractions containing the desired material combined and concentrated in vacuo to reveal the desired product (0.235 g, 45% yield) as a white solid. ¹H NMR (400 MHz, DMSO-d6): δ 8.00-7.77 (m, 4H), 7.50-7.24 (m, 5H), 5.82 (dd, J=9.0, 3.8 Hz, 1H), 4.03-3.92* (m, 2H), 4.05-3.88* (m, 1H), 3.81 (dd, J=14.3, 3.9 Hz, 1H), 3.76-3.58 (m, 2H), 2.23-2.04 (m, 2H). *overlapping peaks. LCMS-B: rt. 3.721; m/z 349.1 [M−H]⁻

### c) 2-amino-1-phenylethyl azetidine-1-carboxylate (A36)

To a suspension of 2-(1,3-dioxoisoindolin-2-yl)-1-phenylethyl azetidine-1-carboxylate (A35) (0.230 g, 0.656 mmol) in EtOH (12 mL) was added hydrazine hydrate (50-60%, 0.123 mL, 1.97-2.36 mmol). The solution was heated to 80° C. for 3 h, upon which time it was cooled and the precipitate filtered. The precipitate was washed with a portion of cold EtOH (5 mL), and the combined EtOH fractions were pooled and concentrated in vacuo to reveal the product (0.122 g, 84% yield) as an oil. The material was carried forward without further purification. LCMS-B: rt. 3.079; no product ion detectable.

### d) 2-(1,1-dioxido-2H-benzo[e][1,2,4]thiadiazine-3-carboxamido)-1-phenylethyl azetidine-1-carboxylate (51)

To 2-amino-1-phenylethyl azetidine-1-carboxylate (A36) (0.050 g, 0.227 mmol) in EtOH (0.125 mL) was added ethyl 2H-benzo[e][1,2,4]thiadiazine-3-carboxylate 1,1-dioxide (12) (0.048 g, 0.189 mmol). The mixture was subjected to microwave irradiation at 100° C. for 30 min. The reaction was cooled and the solvent evaporated. The crude material was purified by silica gel chromatography (Isolera Biotage, 12 g SiO₂ Cartridge, 0-100% EtOAc in petroleum benzine 40-60° C.). The fractions were combined and concentrated to dryness. The material was dissolved in a 1:1:1 mixture of THF: MeOH: 2M NaOH (3 mL) and allowed to stir overnight at r.t. The volatile solvents were removed and the aqueous layer was extracted with EtOAc (3×3 mL). This material was purified by column chromatography (Isolera Biotage, 12 g SiO₂ Cartridge, 0-100% EtOAc in petroleum benzine 40-60° C., then 0-40% MeOH in EtOAc). The fractions containing the desired product were combined and concentrated in vacuo to reveal the product (0.015 g, 19% yield) as a white solid. ¹H NMR (400 MHz, DMSO-d6): δ 12.69 (brs, 1H), 9.27 (brs, 1H), 7.88-7.65 (m, 3H), 7.55-7.47 (m, 1H), 7.45-7.27 (m, 5H), 5.80 (dd, J=8.7, 4.0 Hz, 1H), 4.12-3.95* (m, 2H), 3.91-3.75* (m, 2H), 3.71-3.61* (m, 2H), 2.16 (p, J=7.8, 7.8, 7.7, 7.7 Hz, 2H). *overlapping peaks. LCMS-B: rt. 3.521; m/z 427.1 [M−H]⁻.

### Example 52: N-(2-phenyl-2-(1H-1,2,3-triazol-1-yl)ethyl)-2H-benzo[e][1,2,4]thiadiazine-3-carboxamide 1,1-dioxide (52)

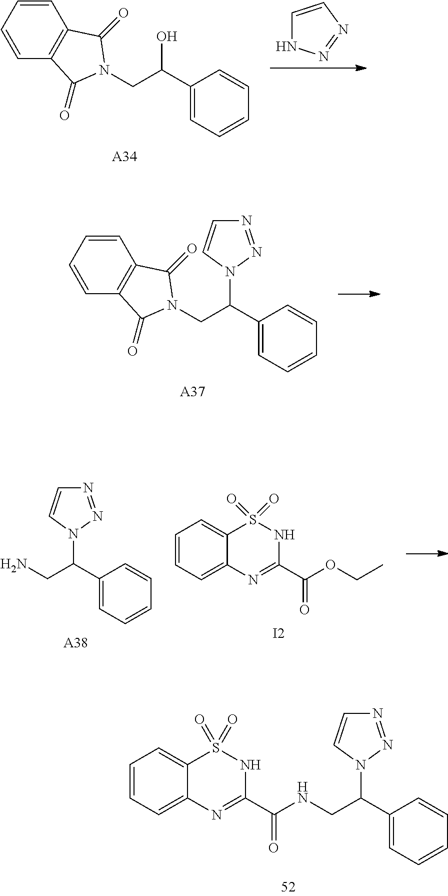

### a) 2-(2-phenyl-2-(1H-1,2,3-triazol-1-yl)ethyl)isoindoline-1,3-dione (A37)

2-(2-Hydroxy-2-phenylethyl)isoindoline-1,3-dione (A34) (0.500 g, 1.871 mmol), 1,2,3-triazole (0.130 mL, 2.245 mmol) and triphenylphosphine (0.589 g, 2.245 mmol), under an atmosphere of nitrogen, were dissolved in THF (25 mL) and cooled to 0° C. DIAD (0.422 mL, 2.245 mmol) was added dropwise over 10 min. The reaction was sealed, allowed to warm to r.t., then stirred overnight. Water (30 mL) was added to quench the reaction. The mixture was extracted with EtOAc (3×20 mL). The combined organic layers were washed with brine (20 mL), dried (Na₂SO₄) and concentrated in vacuo. The crude material was purified by silica gel chromatography (Isolera Biotage, 40 g, 0-100% EtOAc in petroleum benzine 40-60° C.) to yield the product (0.137 g, 23% yield). ¹H NMR (400 MHz, DMSO-d₆): δ 8.35 (d, J=1.1 Hz, 1H), 7.86-7.81 (m, 4H), 7.70 (d, J=1.0 Hz, 1H), 7.56-7.48 (m, 2H), 7.43-7.33 (m, 3H), 6.20 (dd, J=9.2, 6.1 Hz, 1H), 4.60 (dd, J=14.2, 9.2 Hz, 1H), 4.39 (dd, J=14.2, 6.1 Hz, 1H).

### b) 2-phenyl-2-(1H-1,2,3-triazol-1-yl)ethan-1-amine (A38)

To a suspension of 2-(2-phenyl-2-(1H-1,2,3-triazol-1-yl)ethyl)isoindoline-1,3-dione (A37) (0.137 g, 0.430 mmol) in EtOH (12 mL) was added hydrazine hydrate (50-60%, 0.080 mL, 1.29-1.55 mmol). The solution was heated to 80° C. for 3 h, upon which time it was cooled and the precipitate filtered. The precipitate was washed with a portion of cold EtOH (5 mL), and the combined EtOH fractions were pooled and concentrated in vacuo. The material was suspended in cold EtOH (3 mL) and re-filtered. The filtrate was concentrated in vacuo to reveal the product (0.060 g, 74% yield) as a yellow semi-solid. The material was carried forward without any further purification. ¹H NMR (400 MHz, DMSO-d₆): δ 8.30 (d, J=1.0 Hz, 1H), 7.76 (d, J=1.0 Hz, 1H), 7.39-7.27 (m, 5H), 5.69 (dd, J=9.1, 5.4 Hz, 1H), 3.48 *partially obscured by solvent (dd, J=13.5, 9.2 Hz, 2H), 3.26 *partially obscured by solvent (dd, J=13.5, 5.4 Hz, 2H).

### c) N-(2-phenyl-2-(1H-1,2,3-triazol-1-yl)ethyl)-2H-benzo[e][1,2,4]thiadiazine-3-carboxamide 1,1-dioxide (52)

To 2-phenyl-2-(1H-1,2,3-triazol-1-yl)ethan-1-amine (A38) (0.060 g, 0.319 mmol) in EtOH (0.125 mL) was added ethyl 2H-benzo[e][1,2,4]thiadiazine-3-carboxylate 1,1-dioxide (12) (0.068 g, 0.266 mmol). This was irradiated in a microwave reactor at 100° C. for 30 min. The solution was cooled, then concentrated in vacuo. The residue was taken up in EtOAc (2 mL) and the resulting precipitate filtered. The organic layer was washed with 1 M HCl (2 mL), water (2 mL), brine (2 mL), then dried (Na₂SO₄), filtered and concentrated in vacuo. The crude solid was purified by silica gel chromatography (Isolera Biotage 12 g, 0-100% EtOAc in petroleum benzine 40-60° C.). Product-containing fractions were combined and concentrated in vacuo to give the product (0.025 g, 24% yield) as a white solid. ¹H NMR (400 MHz, DMSO-d6): δ 12.65 (s, 1H), 9.51-9.42 (m, 1H), 8.38 (d, J=1.1 Hz, 1H), 7.84 (dd, J=8.0, 1.5 Hz, 1H), 7.79 (d, J=8.4 Hz, 1H), 7.77 (d, J=1.0 Hz, 1H), 7.72 (t, J=7.9 Hz, 1H), 7.52 (t, J=7.6 Hz, 1H), 7.41 (s, 1H), 7.40 (d, J=2.3 Hz, 2H), 7.38-7.33 (m, 1H), 6.16 (dd, J=9.0, 5.6 Hz, 1H), 4.33 (ddd, J=13.7, 9.0, 6.6 Hz, 1H), 4.06 (dt, J=13.7, 5.5, 5.5 Hz, 1H). LCMS-B: rt. 3.408 min; m/z 397.1 [M+H]⁺.

### Example 53: N-(2-(4-methyloxazol-2-yl)-2-phenylethyl)-2H-benzo[e][1,2,4]thiadiazine-3-carboxamide 1,1-dioxide (53)

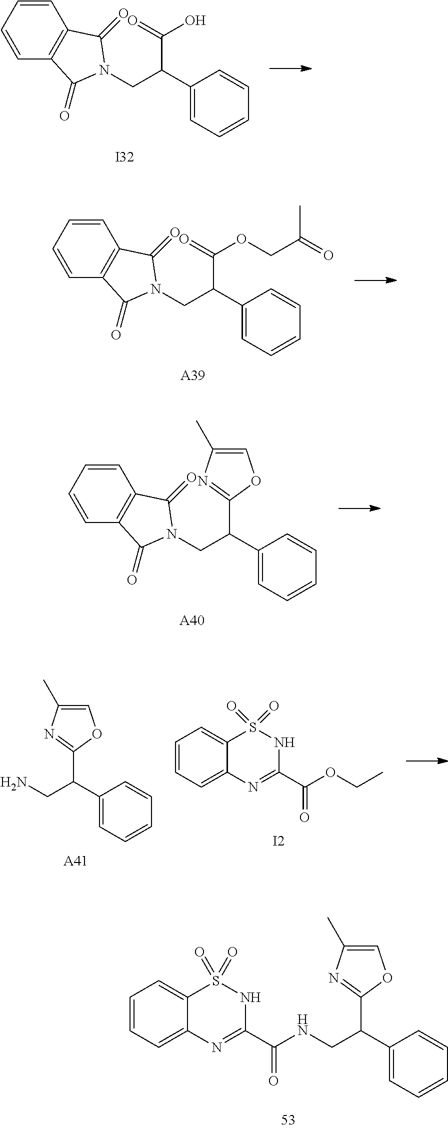

### a) 2-oxopropyl 3-(1,3-dioxoisoindolin-2-yl)-2-phenylpropanoate (A39)

To a solution of 3-(1,3-dioxoisoindolin-2-yl)-2-phenylpropanoic acid (I32) (1.000 g, 3.38 mmol) in THF (5 mL), under an atmosphere of N₂, was added NEt₃ (0.566 mL, 4.06 mmol). The reaction mixture was allowed to stir for 10 min, upon which time it was cooled to 0° C., and chloroacetone (0.419 mL, 5.08 mmol) was added slowly. The mixture was allowed to warm to r.t. and stirred overnight. The formed precipitate was removed by filtration and the filtrate concentrated in vacuo to reveal the product (1.062 g, 89% yield). LCMS-B: rt 3.290 min; no product ion detected.

### b) 2-(2-(4-methyloxazol-2-yl)-2-phenylethyl)isoindoline-1,3-dione (A40)

To a solution of 2-oxopropyl 3-(1,3-dioxoisoindolin-2-yl)-2-phenylpropanoate (A39) (1.062 g, 3.02 mmol) in THF (5 mL), under an atmosphere of nitrogen, was added BF₃.OEt₂ (0.746 mL, 6.05 mmol) followed by acetamide (0.893 g, 15.1 mmol) The mixture was sealed then irradiated in a CEM microwave reactor at 150° C. for 2 h. The reaction mixture was cooled and the solid precipitate filtered. The solid was washed with EtOAc (10 mL) and the combined organics were concentrated in vacuo. The crude material was purified by silica gel chromatography (Isolera Biotage, 40 g Si Cartridge, 0-80% EtOAc in petroleum benzine 40-60° C.). Fractions containing suspected product, eluting at −50% EtOAc, were collected and concentrated in vacuo, to yield the product (0.060 g, 6% yield) as a white solid. LCMS-B: r.t. 3.345 min; m/z 333.1 [M+H]⁺.

### c) 2-(4-methyloxazol-2-yl)-2-phenylethan-1-amine (A41)

To a suspension of 2-(2-(4-methyloxazol-2-yl)-2-phenylethyl)isoindoline-1,3-dione (A40) (0.060 g, 0.18 mmol) in EtOH (4 mL) was added hydrazine hydrate (50-60%, 0.034 mL, 0.55-0.65 mmol). The solution was heated at 80° C. for 17 h. A further portion of hydrazine hydrate (50-60%, 0.034 mL, 0.55-0.65 mmol) was added and the solution allowed to stir for an additional 2 h, upon which time it was cooled and the resulting precipitate filtered. The precipitate was washed with a portion of cold EtOH (5 mL), and the combined EtOH fractions were pooled and concentrated in vacuo to reveal the product (0.022 g, 60% yield). The crude material was carried forward without any further purification. LC-MS: (LCMS-B) r.t. 2.913 min, m/z 203.1 [M+H]⁺.

### d) N-(2-(4-methyloxazol-2-yl)-2-phenylethyl)-2H-benzo[e][1,2,4]thiadiazine-3-carboxamide 1,1-dioxide (53)

To 2-(4-methyloxazol-2-yl)-2-phenylethan-1-amine (A41) (0.022 g, 0.109 mmol) in EtOH (0.125 mL) was added ethyl 2H-benzo[e][1,2,4]thiadiazine-3-carboxylate 1,1-dioxide (12) (0.021 g, 0.084 mmol). The mixture was subjected to microwave irradiation at 100° C. for 30 min. The reaction mixture was cooled and EtOH removed in vacuo. The mixture was taken up in EtOAc (3 mL) and washed with 1 M HCl (3 mL), brine (3 mL), dried (Na₂SO₄) and concentrated in vacuo. The material was further purified by silica gel chromatography (Isolera Biotage, 4 g, 0-100% EtOAc in petroleum benzine 40-60° C.) to give the desired product (0.004 g, 12% yield) as a white solid. ¹H NMR (400 MHz, MeOD): δ 7.94-7.85 (m, 1H), 7.76-7.66 (m, 1H), 7.65-7.57 (m, 1H), 7.58-7.49 (m, 2H), 7.41-7.21 (m, 5H), 4.50-4.43 (m, 1H, partially overlapping with solvent), 4.10-4.00 (m, 1H), 4.00-3.90 (m, 1H), 2.16 (s, 3H), exchangeable NH protons not observed. LCMS-B: rt 3.194 min; m/z 411.1 [M+H]⁺.

### Example 54: N-(2-phenyl-2-(1H-pyrrol-1-yl)ethyl)-2H-benzo[e][1,2,4]thiadiazine-3-carboxamide 1,1-dioxide (54)

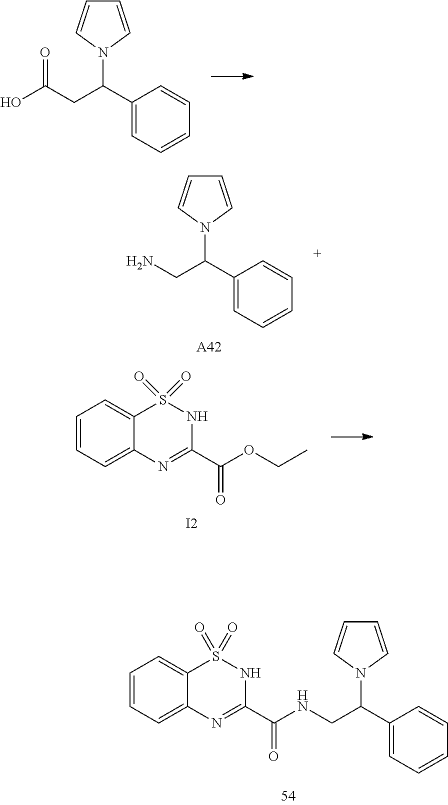

### a) 2-phenyl-2-(1H-pyrrol-1-yl)ethan-1-amine (A42)

To a solution of 3-phenyl-3-(1H-pyrrol-1-yl)propanoic acid (0.300 g, 1.39 mmol) in toluene (6 mL) under an atmosphere of nitrogen was added triethylamine (0.389 mL, 2.79 mmol) and DPPA (0.603 mL, 2.79 mmol). The solution was heated to 80° C., when the evolution of nitrogen began immediately. After 3 h at this temperature, the reaction mixture was cooled to r.t., a 2 M aq. NaOH solution (5 mL) was added and the mixture heated to 80° C. and left to stir overnight. Water (5 mL) was added and the reaction mixture was heated to 110° C., then stirred for a further 17 h. The reaction mixture was concentrated in vacuo and the crude material taken up in minimal MeOH and loaded onto a 10 g SCX cartridge. The cartridge was washed with MeOH (90 mL), then stripped with a 1M solution of methanolic ammonia (90 mL). The ammonia washes were concentrated in vacuo to give the desired product (0.078 g, 30% yield) as a pale yellow oil. ¹H NMR (400 MHz, CDCl₃): δ 7.43-7.28 (m, 3H), 7.25-7.16 (m, 2H), 6.84 (t, J=2.2 Hz, 2H), 6.27 (t, J=2.1 Hz, 2H), 5.10 (dd, J=8.8, 5.8 Hz, 1H), 3.53-3.34 (m, 2H), exchangeable NH protons not observed. LCMS-B: rt. 0.766 min, product mass ion not present.

### b) N-(2-phenyl-2-(1H-pyrrol-1-yl)ethyl)-2H-benzo[e][1,2,4]thiadiazine-3-carboxamide 1,1-dioxide (54)

To 2-phenyl-2-(1H-pyrrol-1-yl)ethan-1-amine (A42) (0.078 g, 0.42 mmol) in EtOH (0.125 mL) was added ethyl 2H-benzo[e][1,2,4]thiadiazine-3-carboxylate 1,1-dioxide (12) (0.071 g, 0.28 mmol). The mixture was subjected to microwave irradiation at 100° C. for 30 min. The reaction mixture was cooled and the resulting precipitate was filtered. The solid was washed with a portion of EtOH (2 mL) and then dried under vacuum to give the desired product (0.069 g, 63% yield) as a grey solid. ¹H NMR (400 MHz, d₆-DMSO): δ 12.92-12.40 (brs, 1H), 9.39-9.34 (dd, J=6.6, 5.1 Hz, 1H), 7.88-7.79 (m, 2H), 7.76-7.68 (m, 1H), 7.57-7.47 (m, 1H), 7.39-7.21 (m, 5H), 6.95-6.90 (t, J=2.1 Hz, 2H), 6.05-5.98 (t, J=2.1 Hz, 2H), 5.64-5.55 (dd, J=9.3, 5.7 Hz, 1H), 4.20-4.05 (ddd, J=13.7, 9.4, 6.7 Hz, 1H), 4.00-3.84 (dt, J=13.8, 5.5 Hz, 1H). LCMS-B: r.t. 3.305 min, m/z 395.1 [M+H]⁺.

### Example 55: N-(2-(2-fluorophenyl)-2-(1H-pyrrol-1-yl)ethyl)-2H-benzo[e][1,2,4]thiadiazine-3-carboxamide 1,1-dioxide (55)

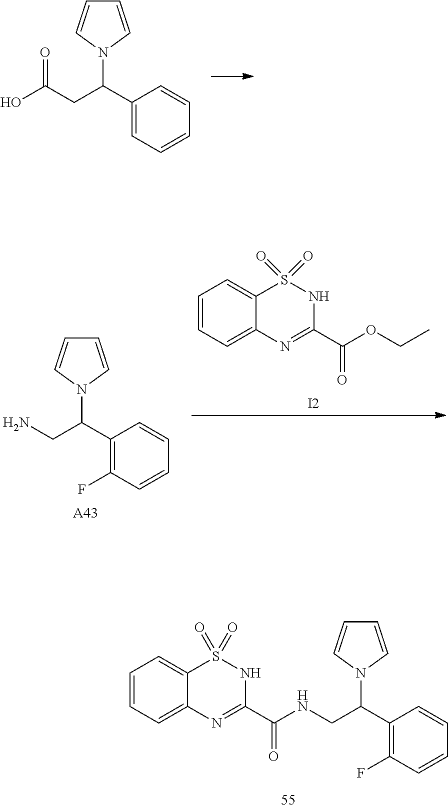

### a) 2-(2-fluorophenyl)-2-(1H-pyrrol-1-yl)ethan-1-amine (A43)

To a solution of 3-phenyl-3-(1H-pyrrol-1-yl)propanoic acid (0.300 g, 1.29 mmol) in toluene (6 mL) under an atmosphere of nitrogen was added triethylamine (0.359 mL, 2.57 mmol) and DPPA (0.556 mL, 2.572 mmol). The solution was heated to 80° C., whereby the evolution of nitrogen began immediately. After 3 h at this temperature, the reaction mixture was cooled to r.t., a 2 M aq. NaOH solution (5 mL) was added and the mixture heated to 80° C. and left to stir overnight. Water (5 mL) was added and the reaction mixture was heated to 110° C., then stirred for a further 17 h. The reaction mixture was concentrated in vacuo and the crude material taken up in minimal MeOH and loaded onto a 10 g SCX cartridge. The cartridge was washed with MeOH (90 mL), then stripped with a solution of methanolic ammonia (90 mL). The ammonia washes were concentrated in vacuo to reveal the desired product (0.135 g, 51% yield) as a pale yellow oil. ¹H NMR (400 MHz, CDCl₃): δ 7.19-7.10 (m, 1H), 7.01-6.87 (m, 3H), 6.73 (t, J=2.1 Hz, 2H), 6.13 (t, J=2.1 Hz, 2H), 5.28 (dd, J=9.1, 5.4 Hz, 1H), 3.40-3.18 (m, 2H), exchangeable NH₂ protons not observed. LCMS-B: rt. 0.774 min, product mass ion not present.

### b) N-(2-(2-fluorophenyl)-2-(1H-pyrrol-1-yl)ethyl)-2H-benzo[e][1,2,4]thiadiazine-3-carboxamide 1,1-dioxide (55)

To 2-(2-fluorophenyl)-2-(1H-pyrrol-1-yl)ethan-1-amine (A43) (0.135 g, 0.661 mmol) in EtOH (0.250 mL) was added ethyl 2H-benzo[e][1,2,4]thiadiazine-3-carboxylate 1,1-dioxide (I2) (0.112 g, 0.441 mmol). The mixture was subjected to microwave irradiation at 100° C. for 30 min. The reaction mixture was cooled and the resulting precipitate was filtered. The solid was washed with a portion of EtOH (2 mL) and then dried under vacuum to reveal the desired product (0.109 g, 60% yield) as a grey solid. ¹H NMR (400 MHz, d₆-DMSO): δ 12.92-12.28 (brs, 1H), 9.49-9.39 (t, J=5.9 Hz, 1H), 7.87-7.78 (m, 2H), 7.76-7.69 (m, 1H), 7.56-7.49 (m, 1H), 7.42-7.28 (m, 2H), 7.27-7.16 (m, 2H), 6.94-6.86 (t, J=2.2 Hz, 2H), 6.04-5.99 (t, J=2.1 Hz, 2H), 5.96-5.88 (dd, J=9.0, 6.0 Hz, 1H), 4.20-4.05 (ddd, J=13.6, 9.2, 6.6 Hz, 1H), 4.00-3.89 (dt, J=13.7, 5.6 Hz, 1H). LCMS-B: r.t. 3.316 min, m/z 413.1 [M+H]⁺.

### Example 56: N-(2-(3-methyl-1,2,4-oxadiazol-5-yl)-2-phenylethyl)-2H-benzo[e][1,2,4]thiadiazine-3-carboxamide 1,1-dioxide (56)

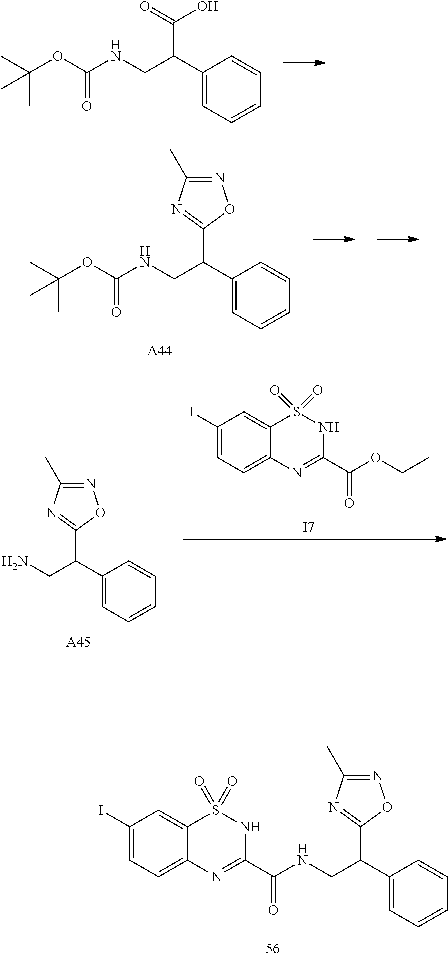

### a) tert-butyl (2-(3-methyl-1,2,4-oxadiazol-5-yl)-2-phenylethyl)carbamate (A44)

To a solution of 3-((tert-butoxycarbonyl)amino)-2-phenylpropanoic acid (1.00 g, 3.7 mmol) in DMF (10 mL), under an atmosphere of nitrogen, was added EDCI.HCl (0.723 g, 3.7 mmol) and HOBt (0.509 g, 3.769 mmol). After 10 min, N-hydroxyacetimidamide (0.279 g, 3.7 mmol) was added. The mixture was allowed to stir at r.t. for 2 h, upon which time the mixture was heated to 80° C. and allowed to stir for 17 h. The reaction mixture was quenched by pouring it into a sat. aq. Na₂CO₃ solution (100 mL). The aqueous layer was extracted with EtOAc (3×100 mL). The combined organics were washed with water (200 mL), brine (200 mL), dried (Na₂SO₄) and concentrated in vacuo. The crude material was purified by column chromatography (Isolera Biotage 40 g, 0-50% EtOAc in petroleum benzine 40-60° C.). Fractions containing the product were combined and concentrated in vacuo to reveal the product (0.475 g, 42% yield) as a white solid. ¹H NMR (400 MHz, CDCl₃): δ 7.41-7.21 (m, 5H, partially obscured by solvent), 4.95 (s, 1H), 4.60-4.44 (m, 1H), 3.76 (t, J=7.1 Hz, 2H), 2.42 (s, 3H), 1.42 (s, 9H). LCMS-F: r.t. 8.968 min, m/z 304.0 [M+H]⁺, 204.0 [M−Boc+H]⁺.

### b) 2-(3-methyl-1,2,4-oxadiazol-5-yl)-2-phenylethan-1-amine (A45)

To tert-butyl (2-(3-methyl-1,2,4-oxadiazol-5-yl)-2-phenylethyl)carbamate (A44) (0.475 g, 1.57 mmol), in DCM (12.5 mL), was added TFA (1.25 mL). The mixture was stirred overnight at r.t. and then diluted with DCM (10 mL), and basified with 2 M NaOH (10 mL). The layers were separated and the aqueous layer washed with further portions of DCM (2×10 mL). The organics were combined, washed with brine (30 mL), dried (Na₂SO₄) and concentrated in vacuo to reveal the product (0.299 g, 94% yield) as a clear oil. ¹H NMR: (400 MHz, CDCl₃): δ 7.36-7.14 (m, 5H), 4.16 (dd, J=7.8, 6.6 Hz, 1H), 3.35 (dd, J=13.1, 7.7 Hz, 1H), 3.20 (dd, J=13.1, 6.6 Hz, 1H), 2.31 (s, 3H), exchangeable NH₂ protons not observed.

### c) N-(2-(3-methyl-1,2,4-oxadiazol-5-yl)-2-phenylethyl)-2H-benzo[e][1,2,4]thiadiazine-3-carboxamide 1,1-dioxide (56)

Ethyl 7-iodo-2H-benzo[e][1,2,4]thiadiazine-3-carboxylate 1,1-dioxide (I7) (0.050 g, 0.13 mmol) and 2-(3-methyl-1,2,4-oxadiazol-5-yl)-2-phenylethan-1-amine (A45) (0.032 g, 0.16 mmol) were suspended in EtOH (0.2 mL), then irradiated in a microwave reactor at 120° C. for 60 min. The mixture was allowed to cool and the precipitate filtered. The precipitate was washed with EtOH (2 mL). The filtrate was concentrated in vacuo then purified by column chromatography (Grace Biotage, 12 g SiO₂, 0-100% EtOAc in petroleum benzines 40-60° C.). Fractions containing the desired product were combined and concentrated in vacuo to reveal the product (0.006 g, 9% yield) as a white solid. ¹H NMR (400 MHz, DMSO-d₆): δ 12.73 (s, 1H), 9.40 (brs, 1H), 8.28-7.93 (m, 2H), 7.58 (d, J=8.8 Hz, 1H), 7.41-7.27 (m, 5H), 4.83 (t, J=7.5 Hz, 1H), 4.09-3.99 (m, 1H), 3.89 (dt, J=13.4, 6.7 Hz, 1H), 2.33 (s, 3H). LC-MS (LCMS-B) r.t. 3.331 min; m/z 537.7 [M+H]⁺.

### Example 57: N-(2-(2-(difluoromethoxy)phenyl)-2-hydroxyethyl)-7-iodo-2H-benzo[e][1,2,4]thiadiazine-3-carboxamide 1,1-dioxide (57)

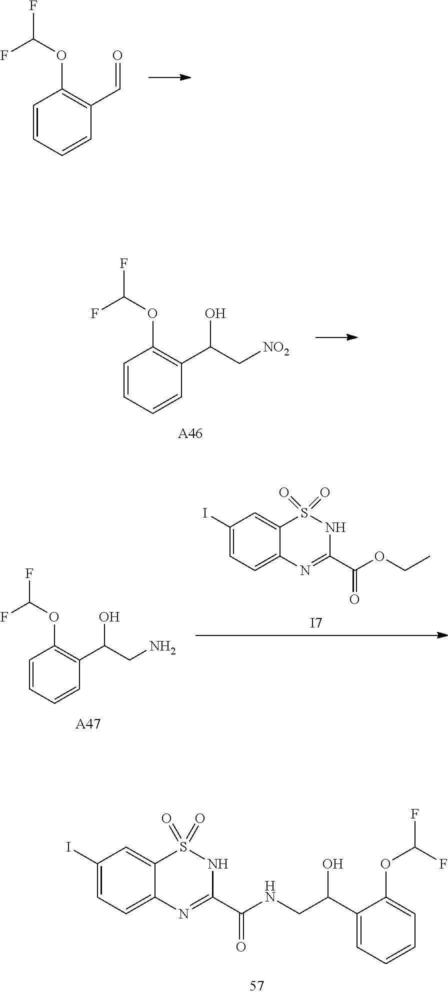

### a) 1-(2-(difluoromethoxy)phenyl)-2-nitroethan-1-ol (A46)

To a solution of 2-(difluoromethoxy)benzaldehyde (2.0 g, 11.7 mmol) in MeOH (25 mL) were added nitromethane (1.88 mL, 34.9 mmol) and sodium methoxide (0.75 g, 13.9 mmol). The solution was allowed to stir for 2 h, then quenched with the addition of 2 M HCl (10 mL) and extracted with EtOAc (30 mL). The organic layer was washed with brine (30 mL×2), dried (Na₂SO₄) and concentrated in vacuo to reveal the product (2.617 g, 97% yield) as an orange oil. The material was carried forward without any further purification. ¹H NMR (400 MHz, CDCl₃): δ 7.62 (dd, J=7.7, 1.8 Hz, 1H), 7.41-7.34 (m, 1H), 7.32-7.20 (m, 1H), 7.14 (ddd, J=8.2, 3.0, 1.1 Hz, 1H), 6.61 (t, J=73.1 Hz, 1H), 5.76 (dd, J=9.1, 3.0 Hz, 1H), 4.85 (dd, J=7.0, 1.0 Hz, 1H), 4.67-4.48 (m, 2H).

### b) 2-amino-1-(2-(difluoromethoxy)phenyl)ethan-1-ol (A47)

1-(2-(Difluoromethoxy)phenyl)-2-nitroethan-1-ol (A46) (1.600 g, 6.862 mmol) and nickel (II) chloride hexahydrate (4.078 g, 17.16 mmol) were dissolved in dry methanol (50 mL) and stirred vigorously under nitrogen. The mixture was cooled to 0° C. and sodium borohydride (6.490 g, 171.5 mmol) was added in 0.5 g portions over 30 min (comment: exothermic, gas evolution). After 1 h, the mixture was quenched with the addition of 2 N HCl (20 mL). The reaction was then basified to ˜pH 11 using sat. NaHCO₃ solution and the MeOH removed in vacuo. EtOAc (50 mL) was added and the layers separated. The aqueous was washed with further portions of EtOAc (3×50 mL). The organics were combined, washed with brine (150 mL), dried (Na₂SO₄) and concentrated in vacuo to reveal the product (1.023 g, 73% yield) as an orange oil. The material was carried forward without any further purification. LCMS-A: r.t. 1.522 min, product mass ion not present.

### c) N-(2-(2-(difluoromethoxy)phenyl)-2-hydroxyethyl)-7-iodo-2H-benzo[e][1,2,4]thiadiazine-3-carboxamide 1,1-dioxide (57)

To 2-amino-1-(2-(difluoromethoxy)phenyl)ethan-1-ol (A47) (0.040 g, 0.20 mmol) in EtOH (0.125 mL) was added ethyl 7-iodo-2H-benzo[e][1,2,4]thiadiazine-3-carboxylate 1,1-dioxide, (I7) (0.050 g, 0.13 mmol). The mixture was subjected to microwave irradiation at 100° C. for 1 h. The reaction mixture was cooled and EtOH removed in vacuo. The reaction mixture was taken up in EtOAc (3 mL) and washed with 1 M HCl (3 mL), brine (3 mL), dried (Na₂SO₄) and concentrated in vacuo to give the product (0.046 g, 65% yield) as a white solid. ¹H NMR (400 MHz, DMSO-d₆) δ 12.75 (brs, 1H), 9.15-8.95 (m, 1H), 8.13-8.02 (m, 2H), 7.65-7.55 (m, 2H), 7.37-7.31 (m, 1H), 7.27 (td, J=7.5, 1.2 Hz, 1H), 7.17 (t, J=73.7 Hz, 1H), 7.19-7.11 (m, 1H), 5.65 (d, J=4.7 Hz, 1H), 5.14 (dt, J=8.6, 4.4 Hz, 1H), 3.48 (dt, J=13.0, 5.1 Hz, 1H), other CH₂ proton obscured by water signal as confirmed by 2D COSY. LCMS-B: r.t. 3.324 min; m/z 535.7 [M−H]⁻.

### Example 58: N-(2-(2-(difluoromethoxy)phenyl)-2-methoxyethyl)-7-iodo-2H-benzo[e][1,2,4]thiadiazine-3-carboxamide 1,1-dioxide (58)

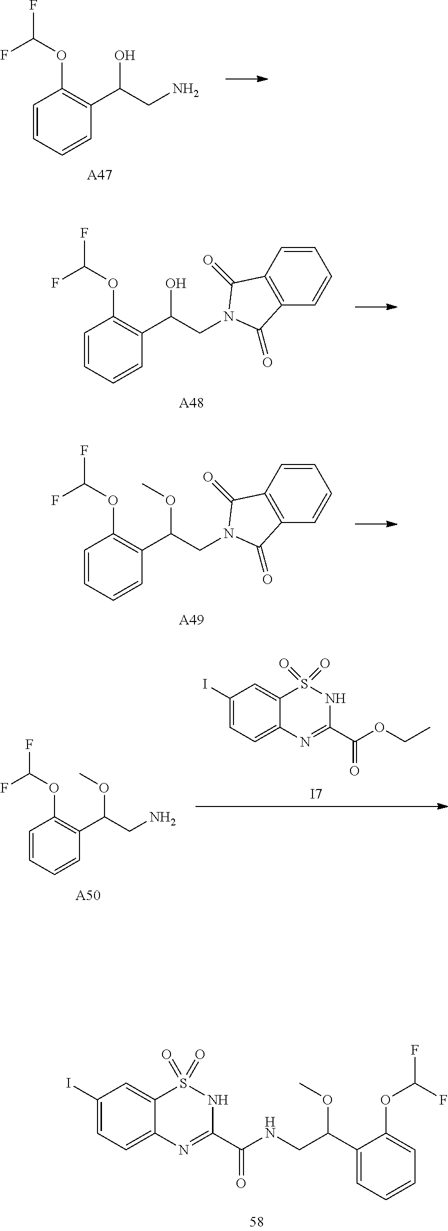

### a) 2-(2-(2-(difluoromethoxy)phenyl)-2-hydroxyethyl)isoindoline-1,3-dione (A48)

2-Amino-1-(2-(difluoromethoxy)phenyl)ethan-1-ol (A47) (0.250 g, 1.23 mmol), phthalic anhydride (0.164 g, 1.1 mmol) and 3 Å molecular sieves were suspended in toluene (10 mL) and the solution heated to 110° C. DMF (1 mL) was added to aid solubility and the reaction was left to stir overnight. The reaction mixture was cooled to r.t., poured into water (50 mL) and then extracted with EtOAc (50 mL). The organic layer was washed with a solution of 1 M HCl (50 mL), brine (50 mL), dried (Na₂SO₄) and concentrated in vacuo to reveal the product (0.245 g, 60% yield) as an orange oil. The material was carried forward without any further purification. ¹H NMR (400 MHz, DMSO-d₆): δ 7.89-7.79 (m, 4H), 7.62 (dd, J=7.6, 1.9 Hz, 1H), 7.36-7.30 (m, 1H), 7.29-7.23 (m, 1H), 7.15 (t, J=74.1 Hz, 1H), 7.13-7.07 (m, 1H), 5.69 (d, J=4.7 Hz, 1H), 5.25 (dt, J=7.9, 5.0 Hz, 1H), 3.79-3.63 (m, 2H).

### b) 2-(2-(2-(difluoromethoxy)phenyl)-2-methoxyethyl)isoindoline-1,3-dione (A49)

To a solution of 2-(2-(2-(difluoromethoxy)phenyl)-2-hydroxyethyl)isoindoline-1,3-dione (A48) (0.245 g, 0.735 mmol) in THF (5 mL) at 0° C., under a nitrogen atmosphere, was added NaH (60% dispersion in mineral oil, 0.044 g, 1.1 mmol). The mixture was allowed to stir for 30 min at this temperature before methyl iodide (0.092 mL, 1.5 mmol) was added. After 30 min at 0° C., the reaction mixture was allowed to warm to r.t. and stirred for 5 h. The reaction mixture was quenched by the addition of water (1 mL) and then the THF was removed in vacuo. The material was partitioned between EtOAc (10 mL) and aq. 1 M HCl (10 mL), then separated. The aqueous layer was further washed with EtOAc (2×10 mL). The organics were combined, washed with brine, dried (Na₂SO₄) and concentrated in vacuo. The crude material was purified by silica gel chromatography (Isolera Biotage, 12 g Si Cartridge, 0-50% EtOAc in petroleum benzine 40-60° C.). Fractions containing suspected product were collected and concentrated in vacuo to yield ˜70% pure material (0.062 g, 24% yield). This impure material was used in the next step without further purification.

### c) 2-(2-(difluoromethoxy)phenyl)-2-methoxyethan-1-amine (A50)

To a suspension of crude 2-(2-(2-(difluoromethoxy)phenyl)-2-methoxyethyl)isoindoline-1,3-dione (A49) (0.062 g, 0.179 mmol) in EtOH (3 mL) was added hydrazine hydrate (50-60%, 0.104 mL, 1.67-2.00 mmol). The solution was stirred at 80° C. overnight, cooled and the precipitate filtered. The precipitate was washed with a portion of cold EtOH (1 mL), and the combined EtOH fractions were pooled and concentrated in vacuo. The resulting solid was re-suspended in minimum cold EtOH, the solid filtered and the EtOH filtrate concentrated in vacuo to reveal the product (0.042 g, >100% yield). The material was carried forward without any further purification. LCMS-A: r.t. 1.678 min, no desired mass ion present.

### d) N-(2-(2-(difluoromethoxy)phenyl)-2-methoxyethyl)-7-iodo-2H-benzo[e][1,2,4]thiadiazine-3-carboxamide 1,1-dioxide (58)

To 2-(2-(difluoromethoxy)phenyl)-2-methoxyethan-1-amine (A50) (0.042 g, 0.193 mmol) in EtOH (0.125 mL) was added ethyl 7-iodo-2H-benzo[e][1,2,4]thiadiazine-3-carboxylate 1,1-dioxide (I7) (0.037 g, 0.097 mmol). The mixture was subjected to microwave irradiation at 100° C. for 1 h. The reaction mixture was cooled and the precipitate filtered. The filtrate was concentrated in vacuo to reveal a complex mixture of products. The crude material was loaded onto a column and purified by silica gel chromatography (Isolera Biotage 4 g Si cartridge, 0-100% EtOAc in petroleum benzine 40-60° C., then 0-40% MeOH in EtOAc). Product-containing fractions were combined and concentrated in vacuo to give the product (0.001 g, 0.5% yield over three steps) as a white solid. LCMS-B: rt. 3.768, m/z 549.7 [M−H]⁻

### Example 59: N-(2-(3-(hydroxymethyl)-1,2,4-oxadiazol-5-yl)-2-phenylethyl)-7-iodo-2H-benzo[e][1,2,4]thiadiazine-3-carboxamide 1,1-dioxide (59)

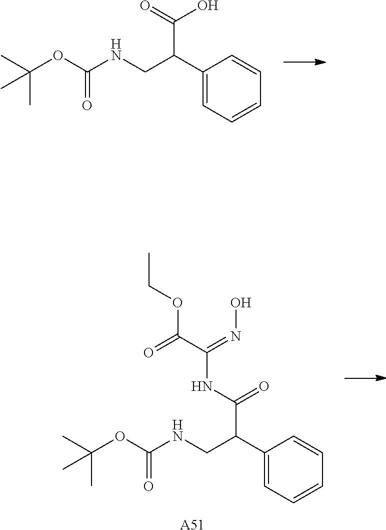 [SMILES: CC(C)(C)OC(=O)NCC(C(=O)O)c1ccccc1 ; CCOC(=O)/C(=N\O)NC(=O)C(CNC(=O)OC(C)(C)C)c1ccccc1 ; "A51"]

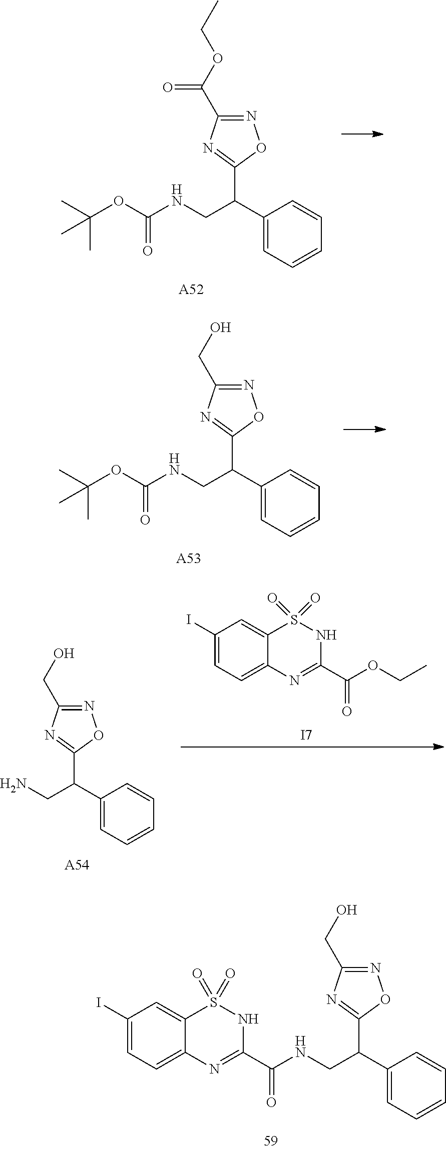

### a) Ethyl (2E)-2-[[3-(tert-butoxycarbonylamino)-2-phenyl-propanoyl]amino]-2-hydroxyimino-acetate (A51)

To 3-{[(tert-Butoxy)carbonyl]amino}-2-phenylpropanoic acid (1.0 g, 3.8 mmol), ethyl 2-(hydroxyamino)-2-imino-acetate (0.50 g, 3.8 mmol) and (2-(7-aza-1H-benzotriazole-1-yl)-1,1,3,3-tetramethyluronium hexafluorophosphate) (1.4 g, 3.8 mmol) in acetonitrile (30 mL) was added N,N-diisopropylethylamine (0.66 mL, 3.8 mmol). This was allowed to stir at r.t. for 1 h, upon which time a white precipitate formed. The mixture was filtered and the resulting solid was washed successively with EtOAc (20 mL), water (50 mL), ether (20 mL), then allowed to air dry to reveal the desired product (1.2 g, 80% yield) as a white solid. ¹H NMR (400 MHz, DMSO-d₆) δ 7.40-7.25 (m, 5H), 7.03 (brs, 2H), 4.25 (q, J=7.1 Hz, 2H), 4.08 (dd, J=8.8, 6.6 Hz, 1H), 3.66-3.53 (m, 1H), 3.30-3.22* partially obscured by solvent (m, 1H), 1.35 (s, 9H), 1.25 (t, J=7.1 Hz, 3H), exchangeable OH proton not observed. LCMS (LCMS-A) rt. 5.691 min; m/z 378.2 [M−H]⁻.

### b) Ethyl 5-[2-(tert-butoxycarbonylamino)-1-phenyl-ethyl]-1,2,4-oxadiazole-3-carboxylate (A52)

Ethyl (2E)-2-[[3-(tert-butoxycarbonylamino)-2-phenyl-propanoyl]amino]-2-hydroxyimino-acetate (A51) (0.85 g, 2.2 mmol) in DMF (5 mL) was heated to 120° C. and allowed to stir o/n. The reaction mixture was cooled and concentrated to dryness. The crude residue was loaded onto a silica gel cartridge and purified by column chromatography (Isolera, Grace 40 g Si cartridge, 0-50% EtOAc in petroleum benzine 40-60° C.) with the material eluting at ˜30% EtOAc collected and concentrated in vacuo to reveal the desired product (430 mg, 53% yield) as a white solid. ¹H NMR: (400 MHz, Chloroform-d): δ 7.36-7.24 *partially obscured by solvent (m, 5H), 5.00 (br t, J=6.4 Hz, 1H), 4.75-4.64 (m, 1H), 4.50 (q, J=7.1 Hz, 2H), 3.89-3.72 (m, 2H), 1.43 (t, J=7.1 Hz, 3H), 1.40 (s, 9H). LCMS-A: rt. 6.006 min; m/z 261.9 [M+H−Boc]⁺.

### c) tert-Butyl N-[2-[3-(hydroxymethyl)-1,2,4-oxadiazol-5-yl]-2-phenyl-ethyl]carbamate (A53)

To a solution of ethyl 5-[2-(tert-butoxycarbonylamino)-1-phenyl-ethyl]-1,2,4-oxadiazole-3-carboxylate (A52) (0.27 g, 0.76 mmol) in EtOH (15 mL) and THF (3 mL), under an atmosphere of nitrogen, was added sodium borohydride (0.057 g, 1.5 mmol). The mixture was allowed to stir o/n at r.t. The reaction mixture was quenched with the addition of aq. 10% citric acid (15 mL). The EtOH and THF were removed in vacuo and EtOAc (15 mL) was added. The layers were separated and the aqueous layer further washed with EtOAc (15 mL). The organic layers were combined, washed with brine, dried (Na₂SO₄) and concentrated in vacuo. The crude material was purified by column chromatography (Grace Biotage, 40 g Si cartridge, 0-100% EtOAc in petroleum benzine 40-60° C.) with the fraction eluting at ˜50% EtOAc identified as the desired product. The fractions containing product were combined and concentrated in vacuo to reveal the desired product (202 mg, 83% yield) as a clear oil. ¹H NMR (400 MHz, Chloroform-d) δ 7.36-7.19*partially obscured by solvent (m, 5H), 5.16 (brt, J=6.4 Hz, 1H), 4.76 (s, 2H), 4.63-4.47 (m, 1H), 3.86-3.69 (m, 2H), 3.66 (s, 1H), 1.39 (s, 9H). LC-MS (LCMS-A): rt. 5.520, m/z 219.9 [M+H−Boc]⁺

### d) [5-(2-amino-1-phenyl-ethyl)-1,2,4-oxadiazol-3-yl]methanol (A54)

tert-Butyl N-[2-[3-(hydroxymethyl)-1,2,4-oxadiazol-5-yl]-2-phenyl-ethyl]carbamate (A53) (0.20 g, 0.63 mmol) was dissolved in DCM (3 mL) and TFA (0.3 mL) was added. This was allowed to stir at r.t. for 2 h. Aqueous 1 M NaOH (1 mL) was added and the organic layer was separated, washed with brine (1 mL), dried (Na₂SO₄) and concentrated in vacuo to give the desired product (0.058 g, 42% yield) as a clear oil. ¹H NMR (400 MHz, Chloroform-d) δ 7.41-7.28 (m, 5H), 4.78 (s, 2H), 4.31 (dd, J=7.6, 6.7 Hz, 1H), 3.47 (dd, J=13.1, 7.7 Hz, 1H), 3.33 (dd, J=13.1, 6.7 Hz, 1H), 1.78 (brs, 3H). LCMS-A:: rt 1.419 min; m/z 219.9 [M+H]⁺.

### e) N-(2-(3-(hydroxymethyl)-1,2,4-oxadiazol-5-yl)-2-phenylethyl)-7-iodo-2H-benzo[e][1,2,4]thiadiazine-3-carboxamide 1,1-dioxide (59)

To a solution of [5-(2-amino-1-phenyl-ethyl)-1,2,4-oxadiazol-3-yl]methanol (A54) 0.035 g, 0.16 mmol) in EtOH (0.125 mL) was added triethylamine (0.022 mL, 0.16 mmol). This was allowed to stir for 10 min, upon which ethyl 7-iodo-2H-benzo[e][1,2,4]thiadiazine-3-carboxylate 1,1-dioxide (I7) (0.050 g, 0.13 mmol) was added. The mixture was irradiated in a microwave reactor at 120° C. for 1 h. The ethanol was removed and the material taken up in EtOAc (3 mL). This was washed with 1 M HCl (3 mL), brine (3 mL), dried (Na₂SO₄) and concentrated in vacuo. The residue was purified by column chromatography (Grace Biotage, 4 g Si cartridge, 0-100% EtOAc in petroleum benzine 40-60° C.) with the fraction eluting at ˜80% EtOAc identified as the desired product. The fraction was concentrated in vacuo though not completely pure by ¹H NMR analysis. The resulting solid was washed with warm EtOAc (0.25 mL), warm DCM (0.25 mL), then air dried give the product (0.0025 g, 2.8% yield) as a white solid. ¹H NMR (400 MHz, DMSO-d6) δ 12.73 (brs, 1H), 9.31 (brs, 1H), 8.11-7.89 (m, 2H), 7.62-7.21 (m, 6H), 5.68 (t, J=6.2 Hz, 1H), 4.86 (t, J=7.6 Hz, 1H), 4.53 (d, J=6.2 Hz, 2H), 3.90 (dt, J=13.5, 6.7 Hz, 2H). LCMS-A:: rt 5.449 min; m/z 551.9 [M−H]⁻.

### Example 60: N-(2-(2H-1,2,3-triazol-2-yl)phenethyl)-7-iodo-2H-benzo[e][1,2,4]thiadiazine-3-carboxamide 1,1-dioxide (60)

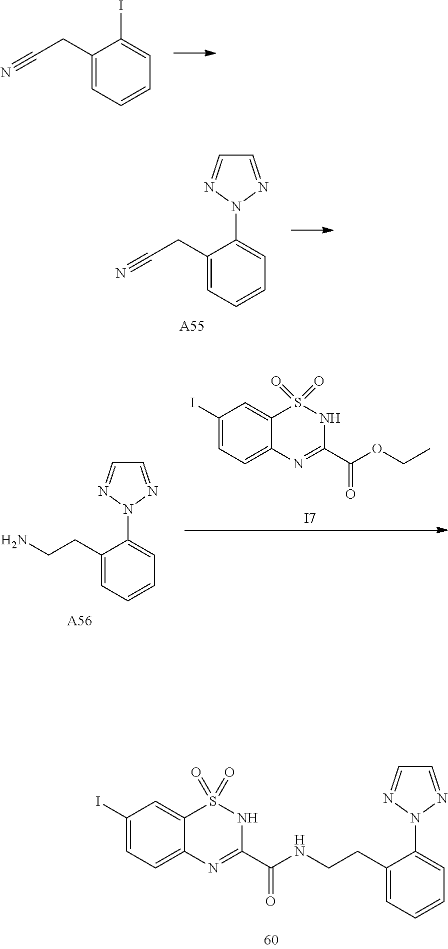

### a) 2-[2-(triazol-2-yl)phenyl]acetonitrile (A55)

To iodophenylacetonitrile (0.57 mL, 4.1 mmol) in DMF (5 mL), under an atmosphere of nitrogen, was added successively, cesium carbonate (60-80 mesh, 2.7 g, 8.2 mmol), copper(I) iodide (0.078 g, 0.41 mmol), triazole (0.48 mL, 8.2 mmol) and dimethylethylenediamine (0.089 mL, 0.82 mmol). The mixture was irradiated in a microwave reactor for 40 min at 100° C. The reaction mixture was cooled and poured into water (75 mL) and extracted with EtOAc (3×75 mL). The organics were combined and washed with brine (200 mL), dried (Na₂SO₄) and concentrated in vacuo. The crude material was purified by column chromatography (Biotage Isolera, 120 g Si cartridge, 0-50% EtOAc in petroleum benzine 40-60° C.) with the material eluting at ˜25% EtOAc identified as the desired material. The fractions containing the material were combined and concentrated in vacuo to give the product (0.10 g, 13% yield) as a white solid. ¹H NMR (400 MHz, CDCl₃) δ 7.87 (s, 2H), 7.82-7.78 (m, 1H), 7.64-7.59 (m, 1H), 7.46 (pd, J=7.4, 1.7 Hz, 2H), 4.08 (s, 2H).

### b) 2-[2-(triazol-2-yl)phenyl]ethanamine (A56)

To 2-[2-(triazol-2-yl)phenyl]acetonitrile (A55) (0.10 g, 0.54 mmol) in THF (5 mL) was added borane-tetrahydrofuran complex (1.0 M solution in THF, 2.7 mL, 2.7 mmol) dropwise. The solution was heated to reflux and allowed to stir o/n. The reaction mixture was cooled and quenched slowly with water (5 mL). A 50% w/v aq. NaOH solution (2 mL) was added and the mixture was refluxed for 1 h. The reaction was cooled and the organics concentrated in vacuo. The remaining aqueous layer was washed with DCM (5 mL×2). The organics were combined, washed with brine (10 mL), dried (Na₂SO₄) and concentrated in vacuo. The crude material was loaded onto an SCX cartridge (1 g) and the column was washed with MeOH (10 mL), then a methanolic ammonia solution (10 mL). The methanolic ammonia washings were concentrated in vacuo leaving the product (0.074 g, 72% yield) as a brown oil. ¹H NMR (400 MHz, CDCl₃) δ 7.80 (s, 2H), 7.55-7.48 (m, 1H), 7.40-7.28 (m, 3H), 2.79 (s, 4H).

### c) N-(2-(2H-1,2,3-triazol-2-yl)phenethyl)-7-iodo-2H-benzo[e][1,2,4]thiadiazine-3-carboxamide 1,1-dioxide (60)

To 2-[2-(triazol-2-yl)phenyl]ethanamine (A56) (0.030 g, 0.16 mmol) in EtOH (0.125 mL) was added ethyl 7-iodo-2H-benzo[e][1,2,4]thiadiazine-3-carboxylate 1,1-dioxide (I7) (0.050 g, 0.13 mmol). This was irradiated in a microwave reactor at 120° C. for 1 h. The reaction mixture was cooled and concentrated to dryness. The residue was taken up in EtOAc (1 mL) and washed with 1 M HCl (1 mL), brine (1 mL), dried (Na₂SO₄) and concentrated in vacuo. The residue was taken up in minimal warm EtOH (0.2 mL) and allowed to slowly cool. The resulting solid was collected and air dried to reveal the desired product N-(2-(2H-1,2,3-triazol-2-yl)phenethyl)-7-iodo-2H-benzo[e][1,2,4]thiadiazine-3-carboxamide 1,1-dioxide (0.0070 g, 10% yield) as a white solid. ¹H NMR (400 MHz, DMSO-d6) δ 12.68 (s, 1H), 9.29 (t, J=5.9 Hz, 1H), 8.09 (s, 2H), 8.12-8.03 (m, 2H), 7.60 (d, J=8.6 Hz, 1H), 7.56-7.47 (m, 3H), 7.47-7.40 (m, 1H), 3.47-3.39* partially obscured by solvent (m, 2H), 2.91 (t, J=7.2 Hz, 2H). LCMS-B: rt. 3.319 min; m/z 520.7 [M−H]⁻.

General Methods

Method A:

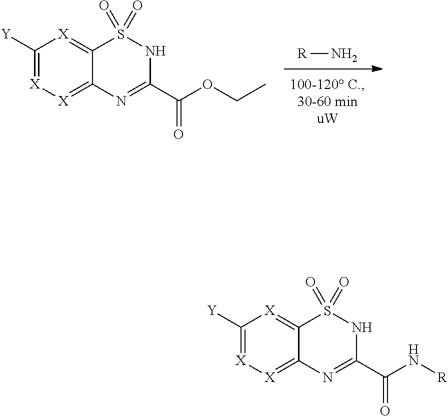

To a solution of the amine (1.2 eq.) in EtOH (0.8 M) was added the ester (1 eq.). This was irradiated in a microwave reactor for 30 min at 100° C. The reaction mixture was cooled and the resulting precipitate filtered, washed with cold EtOH, then air dried to give the desired product.
A-1: Reaction temperature increased to 120° C.; reaction time extended to 1 h
A-2: Reaction temperature increased to 120° C.; reaction time extended to 2 h
A-3: Additional EtOH wash of solid required to remove residual impurities
A-4: Column chromatography of isolated material required

Method B:

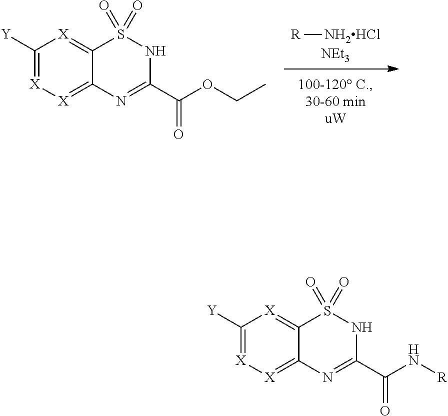

To a solution of the amine (1.2 eq.) in EtOH (0.8 M) was added triethylamine (1.2 eq.). After 10 min the ester (1 eq.) was added and the mixture was irradiated in a microwave reactor for 30 min at 100° C. The reaction mixture was cooled and resulting precipitate filtered, washed with cold EtOH, then air dried to reveal the desired product.
B-1: Reaction time extended to 1 h
B-2: Reaction time extended to 1 h; column chromatography of isolated material required
B-3: Precipitated by cooling to 4° C. overnight
B-4: Reaction produced a mixture of two major products, separated by preparatory TLC in 2% MeOH/DCM

Method C:

To a solution of the amine (1.2 eq.) in EtOH (0.8 M) was added the ester (1 eq.). This was irradiated in a microwave reactor for 30 min at 100° C. The reaction mixture was cooled and the solvent removed. The material was taken up in EtOAc and washed with 1 M HCl, brine, dried and concentrated in vacuo to reveal the desired product.

Method D:

To a solution of the amine (1.2 eq.) in EtOH (0.8 M) was added triethylamine (1.2 eq.). After 10 min the ester (1 eq.) was added and the mixture was irradiated in a microwave reactor for 30 min at 100° C. The reaction mixture was cooled and the solvent removed. The material was taken up in EtOAc and washed with 1 M HCl, brine, dried and concentrated in vacuo to reveal the desired product.

Method E:

To a solution of the ester (1 eq.) and amine (1.5 eq.) in EtOH (0.06 M) was added Et₃N (3 eq.) and the mixture heated at 120° C. in a sealed tube for 3 h. The mixture was concentrated under reduced pressure and the residue was recrystallized from MeOH (2 mL) to afford the desired product.

Method F:

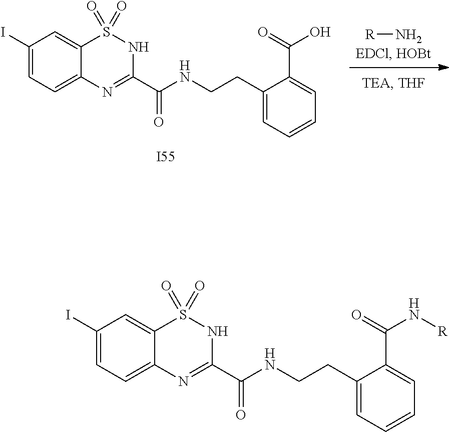

To a solution of the acid 155 (1 eq.), HOBt (1.5 eq.), EDCI.HCl (2 eq.) and triethylamine (3 eq.) in THF (0.02 M) was added the amine (1.5 eq.) and the mixture was stirred at r.t. for 16 h. Water (5 mL) was added and the mixture extracted with EtOAc (8 mL×3). The combined organic extracts were dried over Na₂SO₄ and concentrated. The residue was purified by preparative TLC (DCM/MeOH=10:1) to give the desired product.

Method G:

A suspension of the ester (1 eq.), amine (1 eq.) and Et₃N (2-4 eq.) in EtOH (0.8 M) was irradiated in the microwave at 150° C. for 30 min. Upon cooling, water (1 mL) and diethyl ether (5 mL) were added and the mixture sonicated for 10 min. The resulting precipitates were collected by filtration and air dried to yield the desired compounds.
G-1: The precipitate was treated with LiOH-hydrate (217 mg) in THF:MeOH:water 10:1:0.5 at room temperature overnight and purified by column chromatography (0-100% EtOAc/hexanes, then 0-40% MeOH in EtOAc).
G-2: Heated at 100° C. for 30 min; precipitated by adding petroleum benzene

| Example | Name & Structure | LCMS data | | Method |
|---|---|---|---|---|
| 61 | 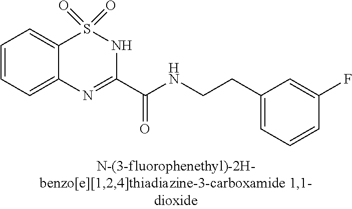 [SMILES: O=C(NCCc1cccc(F)c1)C1=Nc2ccccc2S(=O)(=O)N1] | LC-MS B: rt. 3.580 min, m/z 348.2 [M + H]+ | | A |
| 62 | 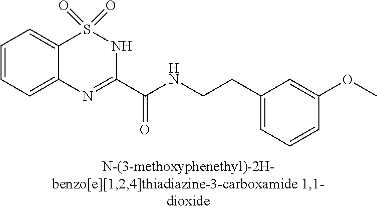 [SMILES: COc1cccc(CCNC(=O)C2=Nc3ccccc3S(=O)(=O)N2)c1] | LCMS B: rt. 3.556; m/z 360.2 [M + H]+ | | A |
| 63 | 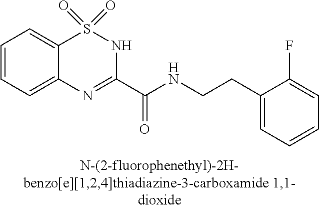 [SMILES: O=C(NCCc1ccccc1F)C1=Nc2ccccc2S(=O)(=O)N1] | LCMS B: rt. 3.575; m/z 348.1 [M + H]+ | | A |
| 64 | 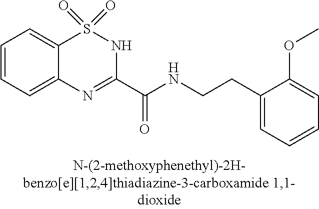 [SMILES: COc1ccccc1CCNC(=O)C1=Nc2ccccc2S(=O)(=O)N1] | LCMS B: rt. 3.605; m/z 360.2 [M + H]+ | | A |
| 65 | 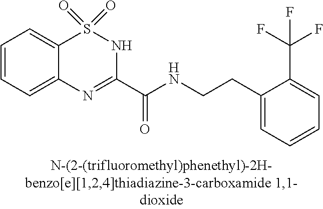 [SMILES: O=C(NCCc1ccccc1C(F)(F)F)C1=Nc2ccccc2S(=O)(=O)N1] | LCMS B: rt. 3.682; m/z 396.1 [M − H]− | | A |
| 66 | 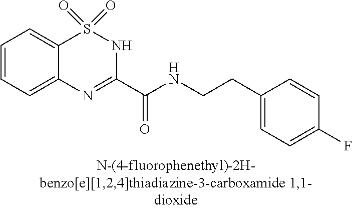 [SMILES: O=C(NCCc1ccc(F)cc1)C1=Nc2ccccc2S(=O)(=O)N1] | LCMS B: rt. 3.573; m/z 348.1 [M + H]+ | | A |
| 67 | 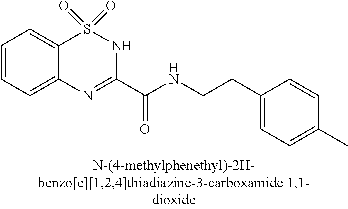 [SMILES: Cc1ccc(CCNC(=O)C2=Nc3ccccc3S(=O)(=O)N2)cc1] | LCMS B: rt. 3.637 min; m/z 344.2 [M + H]+ | | A |
| 68 | 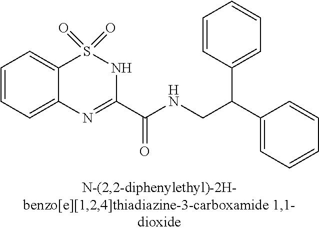 [SMILES: O=C(NCC(c1ccccc1)c1ccccc1)C1=Nc2ccccc2S(=O)(=O)N1] | LCMS B: rt. 3.709 min; m/z 406.2 [M + H]+ | | A |
| 69 | 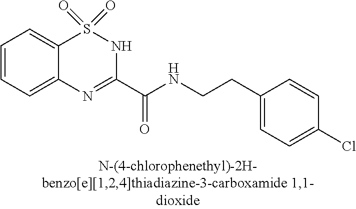 [SMILES: O=C(NCCc1ccc(Cl)cc1)C1=Nc2ccccc2S(=O)(=O)N1] | LCMS B: rt. 3.709 min; m/z 362.2 [M − H]− | | A |
| 70 | 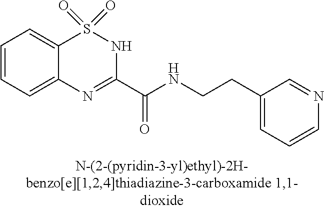 [SMILES: O=C(NCCc1cccnc1)C1=Nc2ccccc2S(=O)(=O)N1] | LCMS B: rt. 3.133 min; m/z 331.1 [M + H]+ | | A |
| 71 | 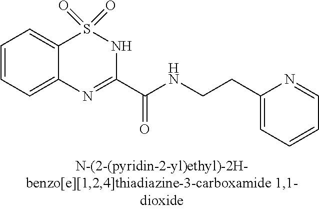 [SMILES: O=C(NCCc1ccccn1)C1=Nc2ccccc2S(=O)(=O)N1] | LCMS B: rt. 3.139 min; m/z 331.1 [M + H]+ | | A |
| 72 | 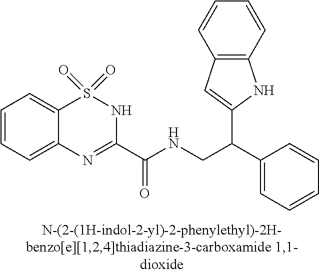 [SMILES: O=C(NCC(c1ccccc1)c1cc2ccccc2[nH]1)C1=Nc2ccccc2S(=O)(=O)N1] | LCMS B: rt. 3.659 min; m/z 444.1 [M + H]+ | | A |
| 73 | 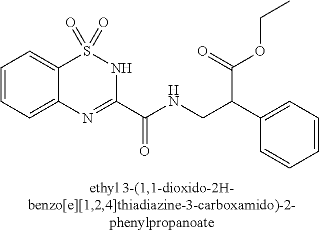 [SMILES: CCOC(=O)C(CNC(=O)C1=Nc2ccccc2S(=O)(=O)N1)c1ccccc1] | LCMS B: rt. 3.555 min, m/z 402.2 [M + H]+. | | B |
| 74 | 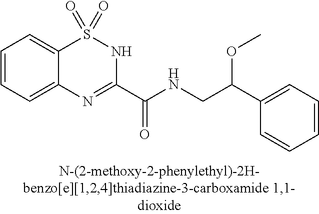 [SMILES: COC(CNC(=O)C1=Nc2ccccc2S(=O)(=O)N1)c1ccccc1] | LCMS B: rt. 3.525 min, m/z 358.1 [M − H]−. | | A |
| 75 | 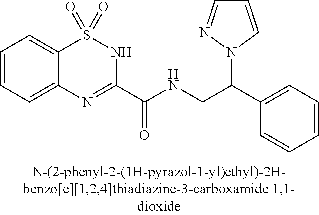 [SMILES: O=C(NCC(c1ccccc1)n1cccn1)C1=Nc2ccccc2S(=O)(=O)N1] | LCMS B: rt. 3.489 min, m/z 396.2 [M + H]. | | C |
| 76 | 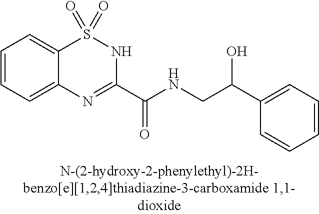 [SMILES: O=C(NCC(O)c1ccccc1)C1=Nc2ccccc2S(=O)(=O)N1] | LCMS B: rt. 3.378 min, m/z 344.1 [M − H]−. | | C |
| 77 | 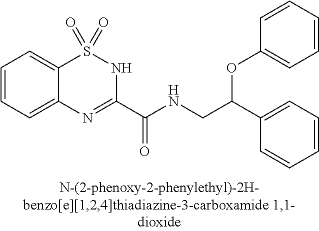 [SMILES: O=C(NCC(Oc1ccccc1)c1ccccc1)C1=Nc2ccccc2S(=O)(=O)N1] | LCMS B: rt. 3.693 min, m/z 420.1 [M − H]−. | | B |
| 78 | 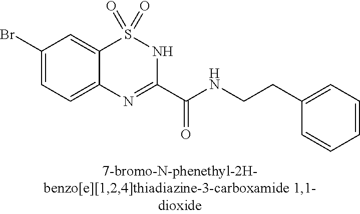 [SMILES: O=C(NCCc1ccccc1)C1=Nc2ccc(Br)cc2S(=O)(=O)N1] | LCMS B: rt. 3.320 min; m/z 408.0 [M]+. | | A |
| 79 | 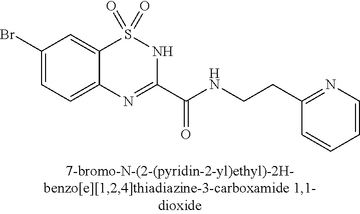 [SMILES: O=C(NCCc1ccccn1)C1=Nc2ccc(Br)cc2S(=O)(=O)N1] | LC-MS B: rt. 2.791 min; m/z 408.8 [M + H]+ | | A-3 |
| 80 | 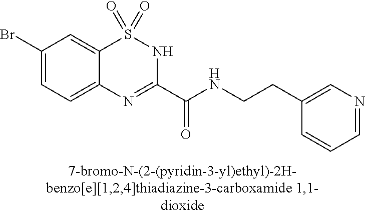 [SMILES: O=C(NCCc1cccnc1)C1=Nc2ccc(Br)cc2S(=O)(=O)N1] | LCMS B: rt. 2.779 min; m/z 408.8 [M + H]+ | | A-3 |
| 81 | 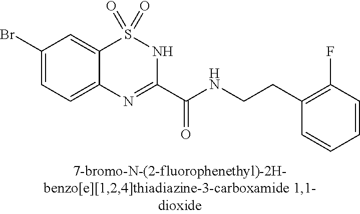 [SMILES: O=C(NCCc1ccccc1F)C1=Nc2ccc(Br)cc2S(=O)(=O)N1] | LCMS B: rt. 3.419 min; m/z 425.8 [M − H]− | | A |
| 82 | 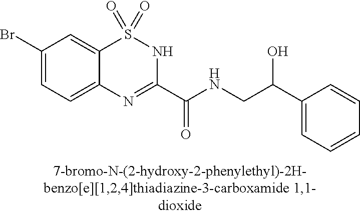 [SMILES: O=C(NCC(O)c1ccccc1)C1=Nc2ccc(Br)cc2S(=O)(=O)N1] | LCMS B: rt. 3.245 min; m/z 421.7 [M − H]− | | A |
| 83 | 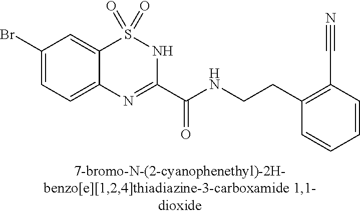 [SMILES: N#Cc1ccccc1CCNC(=O)C1=Nc2ccc(Br)cc2S(=O)(=O)N1] | LCMS B: rt. 3.342 min; m/z 433.8 [M + H]+ | | B |
| 84 | 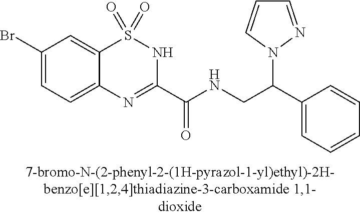 [SMILES: O=C(NCC(c1ccccc1)n1cccn1)C1=Nc2ccc(Br)cc2S(=O)(=O)N1] | LCMS B: rt. 3.338 min; m/z 473.8 [M + H]+ | | A |
| 85 | 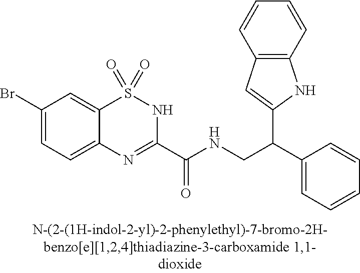 [SMILES: O=C(NCC(c1ccccc1)c1cc2ccccc2[nH]1)C1=Nc2ccc(Br)cc2S(=O)(=O)N1] | LCMS B: rt. 3.485 min; m/z 442.8 [M − Br]− | | A |
| 86 | 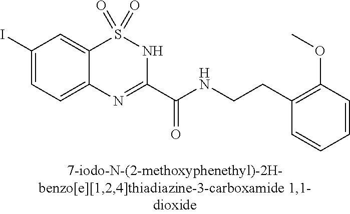 [SMILES: COc1ccccc1CCNC(=O)C1=Nc2ccc(I)cc2S(=O)(=O)N1] | LCMS B: rt. 3.411 min; m/z 483.7 [M − H]− | | A-1 |
| 87 | 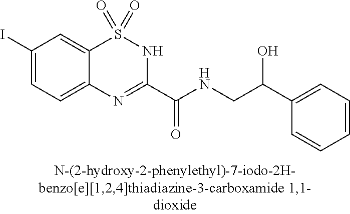 [SMILES: O=C(NCC(O)c1ccccc1)C1=Nc2ccc(I)cc2S(=O)(=O)N1] | LCMS B: rt. 3.217 min; m/z 469.7 [M − H]− | | A-2 |
| 88 | 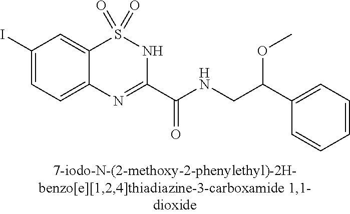 [SMILES: COC(CNC(=O)C1=Nc2ccc(I)cc2S(=O)(=O)N1)c1ccccc1] | LCMS B: rt. 3.380 min; m/z 483.7 [M − H]− | | A-1 |
| 89 | 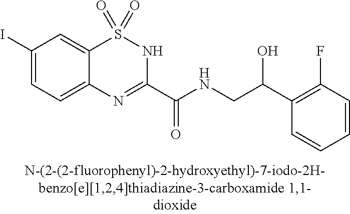 [SMILES: O=C(NCC(O)c1ccccc1F)C1=Nc2ccc(I)cc2S(=O)(=O)N1] | LCMS B: rt. 3.250 min; m/z 487.7 [M − H]− | | A-2 |
| 90 | 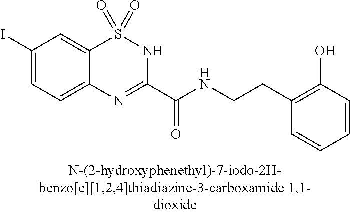 [SMILES: O=C(NCCc1ccccc1O)C1=Nc2ccc(I)cc2S(=O)(=O)N1] | LCMS B: rt. 3.265 min; m/z 469.7 [M − H]− | | B-1 |
| 91 | 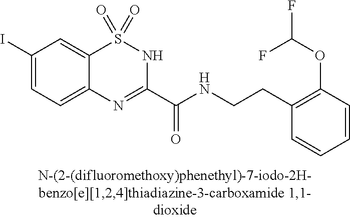 [SMILES: O=C(NCCc1ccccc1OC(F)F)C1=Nc2ccc(I)cc2S(=O)(=O)N1] | LCMS B: rt. 3.403 min; m/z 519.7 [M − H]− | | B-2 |
| 92 | 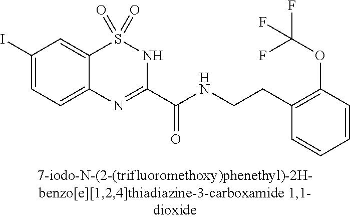 [SMILES: O=C(NCCc1ccccc1OC(F)(F)F)C1=Nc2ccc(I)cc2S(=O)(=O)N1] | LCMS B: rt. 3.482 min; m/z 537.7 [M − H]− | | B-1 |
| 93 | 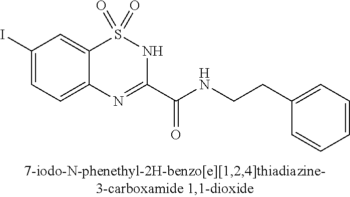 [SMILES: O=C(NCCc1ccccc1)C1=Nc2ccc(I)cc2S(=O)(=O)N1] | LCMS B: rt. 3.371 min; m/z 453.7 [M − H]−. | | A-1 |
| 94 | 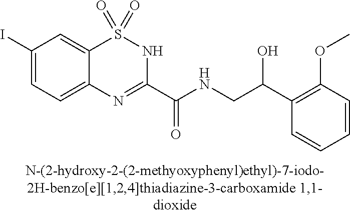 [SMILES: COc1ccccc1C(O)CNC(=O)C1=Nc2ccc(I)cc2S(=O)(=O)N1] | LCMS B: rt. 3.308 min; m/z 499.7 [M − H]−. | | A-1 |
| 95 | 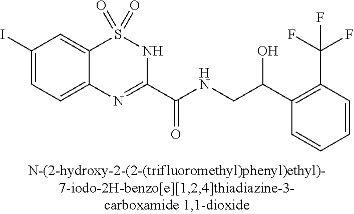 [SMILES: O=C(NCC(O)c1ccccc1C(F)(F)F)C1=Nc2ccc(I)cc2S(=O)(=O)N1] | LCMS B: rt. 3.382 min; m/z 537.7 [M − H]−. | | A-1 |
| 96 | 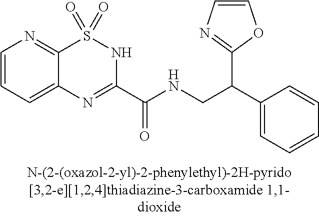 [SMILES: O=C(NCC(c1ccccc1)c1ncco1)C1=Nc2cccnc2S(=O)(=O)N1] | LCMS B: rt. 3.137 min, m/z 398.1 [M + H]⁺. | | A-4 |
| 97 | 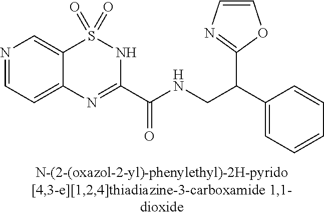 [SMILES: O=C(NCC(c1ccccc1)c1ncco1)C1=Nc2ccncc2S(=O)(=O)N1] | LC-MS B: rt. 3.080 min; m/z 397.8 [M + H]⁺. | | A-1 |
| 98 | 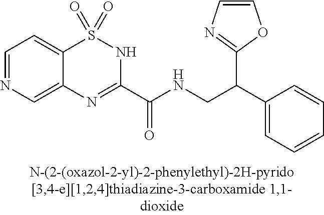 [SMILES: O=C(NCC(c1ccccc1)c1ncco1)C1=Nc2cnccc2S(=O)(=O)N1] | LCMS B: rt. 3.121 min, m/z 397.8 [M + H]⁺. | | A-1 |
| 99 | 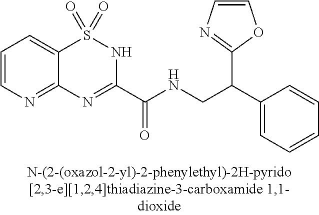 [SMILES: O=C(NCC(c1ccccc1)c1ncco1)C1=Nc2ncccc2S(=O)(=O)N1] | LCMS-A: rt. 4.030 min; m/z 398.3 [M + H]⁺. | | A |
| Example | Name and structure | LCMS data | ¹HNMR data | Method |
| 100 | 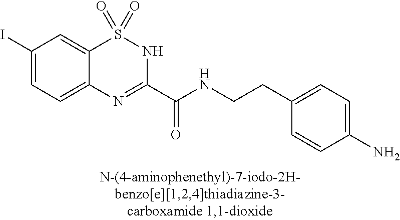 [SMILES: Nc1ccc(CCNC(=O)C2=Nc3ccc(I)cc3S(=O)(=O)N2)cc1] | LCMS (ES- API): Rₜ 2.33 min, m/z 471.0 [M + H]⁺ | ¹H NMR (400 MHz, d₆-DMSO) δ 9.23 (t, J = 6.0 Hz, 1H), 8.09 (d, J = 1.6 Hz, 1H), 8.06 (dd, J = 8.4, 1.6 Hz, 1H), 7.60 (d, J = 8.4 Hz, 1H), 6.87 (d, J = 8.2 Hz, 2H), 6.49 (d, J = 8.2 Hz, 2H), 3.38 (m, 2H), 2.66 (t, J = 7.6 Hz , 2H), exchangeable NH protons not | E |
| | | | observed. | |
| 101 | 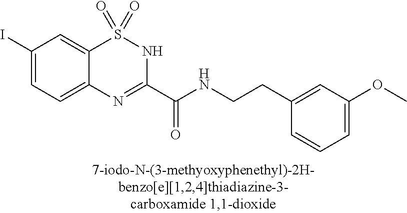 [SMILES: COc1cccc(CCNC(=O)C2=Nc3ccc(I)cc3S(=O)(=O)N2)c1] | LCMS (ES- API): Rₜ 2.83 min, m/z 486.0 [M + H]⁺ | ¹H NMR (400 MHz, d₆-DMSO) δ 12.7 (brs, 1H), 9.28 (t, J = 6.0 Hz, 1H), 8.09 (s, 1H), 8.07 (d, J = 8.8 Hz, 1H), 7.60 (d, J = 8.6 Hz, 1H), 7.20 (t, J = 7.9 Hz, 1H), 6.81-6.75 (m, 3H), 3.72 (s, 3H), 3.53-3.46 (m, 2H), 2.84 (t, J = 7.0 Hz, 2H). | E |
| 102 | 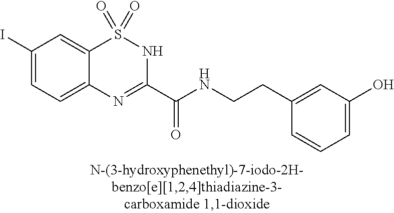 [SMILES: O=C(NCCc1cccc(O)c1)C1=Nc2ccc(I)cc2S(=O)(=O)N1] | LCMS (ES- API): Rₜ 2.67 min, m/z 472.0 [M + H]⁺ | ¹H NMR (400 MHz, d₆-DMSO) δ 12.8 (brs, 1H), 9.33-9.23 (m, 2H), 8.13- 8.00 (m, 2H), 7.58 (m, 1H), 7.07 (m, 1H), 6.71-6.56 (m, 3H), 3.45 (m, 2H), 2.76 (m, 2H). | E |
| 103 | 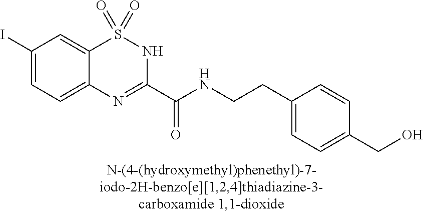 [SMILES: O=C(NCCc1ccc(CO)cc1)C1=Nc2ccc(I)cc2S(=O)(=O)N1] | LCMS (ES- API): Rₜ 2.569 min, m/z 508.0 [M + Na]⁺ | ¹H NMR (400 MHz, d₆-DMSO) δ 12.7 (brs, 1H), 9.27 (t, J = 5.5 Hz, 1H), 8.09 (s, 1H), 8.07 (d, J = 8.8 Hz, 1H), 7.60 (d, J = 8.7 Hz, 1H), 7.23 (d, J = 7.6 Hz, 2H), 7.18 (d, J = 7.6 Hz, 2H), 5.10 (t, J = 5.4 Hz, 1H), 4.45 (d, J = 4.8 Hz, 2H), 3.51-3.45 (m, 2H), 2.84 (t, J = 7.1 | E |
| | | | Hz, 2H). | |
| 104 | 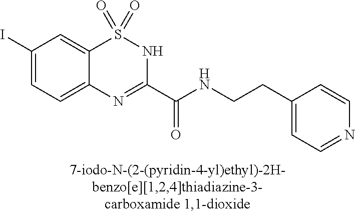 [SMILES: O=C(NCCc1ccncc1)C1=Nc2ccc(I)cc2S(=O)(=O)N1] | LCMS (ES- API): Rₜ 1.106 min, m/z 456.9 [M + H]⁺ | ¹H NMR (400 MHz, d₆-DMSO) δ 12.7 (brs, 1H), 9.32 (t, J = 5.7 Hz, 1H), 8.47 (brs, 2H), 8.13- 8.02 (m, 2H), 7.60 (d, J = 8.7 Hz, 1H), 7.27 (brs, 2H), 3.60-3.48 (m, 2H), 2.89 (t, J = 6.9 Hz, 2H). | E |
| 105 | 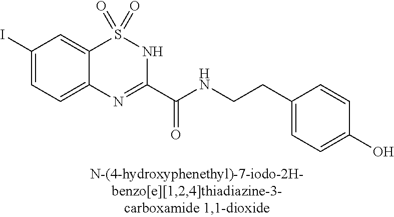 [SMILES: O=C(NCCc1ccc(O)cc1)C1=Nc2ccc(I)cc2S(=O)(=O)N1] | LCMS (ES- API): Rₜ 1.966 min, m/z 472.0 [M + H]⁺ | ¹H NMR (400 MHz, d₆-DMSO) δ 12.7 (s, 1H), 9.24 (t, J = 5.1 Hz, 1H), 9.18 (s, 1H), 8.14-8.03 (m, 2H), 7.61 (d, J = 8.7 Hz, 1H), 7.01 (d, J = 8.1 Hz, 2H), 6.67 (d, J = 8.2 Hz, 2H), 3.45-3.42 (m, 2H), 2.73 (t, J = 7.2 Hz, 2H). | E |
| 106 | 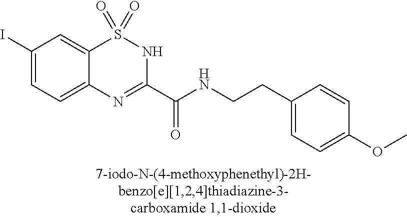 [SMILES: COc1ccc(CCNC(=O)C2=Nc3ccc(I)cc3S(=O)(=O)N2)cc1] | LCMS (ES- API): Rₜ 2.744 min, m/z 486.0 [M + H]⁺ | ¹H NMR (400 MHz, d₆-DMSO) δ 12.7 (s, 1H), 9.26 (t, J = 5.8 Hz, 1H), 8.13- 8.03 (m, 2H), 7.61 (d, J = 8.7 Hz, 1H), 7.14 (d, J = 8.5 Hz, 2H), 6.85 (d, J = 8.5 Hz, 2H), 3.71 (s, 3H), 3.48-334 (m, 2H), 2.79 (t, J = 7.3 Hz, 2H). | E |
| 107 | 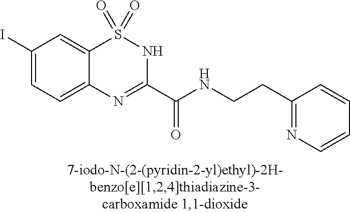 [SMILES: O=C(NCCc1ccccn1)C1=Nc2ccc(I)cc2S(=O)(=O)N1] | LCMS (ES- API): Rₜ 1.399 min, m/z 457.0 [M + H]⁺ | ¹H NMR (400 MHz, d₆-DMSO) δ 12.7 (brs, 1H), 9.32 (t, J = 5.8 Hz, 1H), 8.50 (d, J = 4.8 Hz, 1H), 8.10 (d, J = 2.0 Hz, 1H), 8.07 (dd, J = 8.8, 2.0 Hz, 1H), 7.71 (td, J = 7.6, 2.0 Hz, 1H), 7.61 (d, J = 8.7 Hz, 1H), 7.28 (d, J = 8.0 Hz, 1H), 7.24 (dd, J = 7.6, 5.2 Hz, 1H), | E |
| | | | 3.66-3.61 (m, 2H), | |
| | | | 3.02 (t, J = 7.3 Hz, | |
| | | | 2H). | |
| 108 | 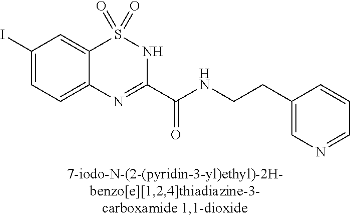 [SMILES: O=C(NCCc1cccnc1)C1=Nc2ccc(I)cc2S(=O)(=O)N1] | LCMS (ES- API): Rₜ 1.272 min, m/z 457.0 [M + H]⁺ | ¹H NMR (400 MHz, d₆-DMSO) δ 12.7 (brs, 1H), 9.32 (t, J = 5.7 Hz, 1H), 8.45 (s, 1H), 8.41 (d, J = 4.4 Hz, 1H), 8.09 (s, 1H), 8.06 (d, J = 8.8 Hz, 1H), 7.66 (d, J = 7.7 Hz, 1H), 7.60 (d, J = 8.7 Hz, 1H), 7.32 (dd, J = 7.6, 4.8 Hz, 1H), 3.55-3.50 (m, 2H), 2.89 (t, J = 6.9 Hz, | E |
| | | | 2H). | |
| 109 | 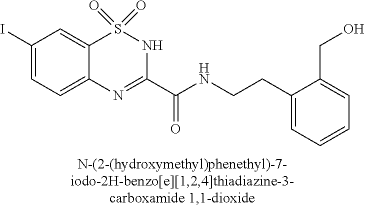 [SMILES: O=C(NCCc1ccccc1CO)C1=Nc2ccc(I)cc2S(=O)(=O)N1] | LCMS (ES- API): Rₜ 2.651 min, m/z 484.0 [M]⁻ | ¹H NMR (400 MHz, d₆-DMSO) δ 12.7 (brs, 1H), 9.34 (t, J J = 5.8 Hz, 1H), 8.18-8.00 (m, 2H), 7.60 (d, J = 8.7 Hz, 1H), 7.43-7.35 (m, 1H), 7.22-7.17 (m, 3H), 5.10 (t, J = 5.2 Hz, 1H), 4.59 (d, J = 4.8 Hz, 2H), 3.47- 3.33 (m, 2H), 2.93- 2.80 (t, J = 7.8 | E |
| | | | Hz, 2H). | |
| 110 | 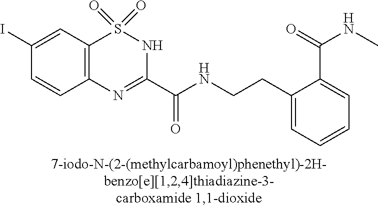 [SMILES: CNC(=O)c1ccccc1CCNC(=O)C1=Nc2ccc(I)cc2S(=O)(=O)N1] | LCMS (ES- API): Rₜ 2.46 min, m/z 512.7 [M + H]⁺ | ¹H NMR (400 MHz, d₆-DMSO) δ 12.6 (brs, 1H), 9.34 (t, J = 4.8 Hz, 1H), 8.21 (m, 1H), 8.09 (s, 1H), 8.06 (d, J = 8.8 Hz, 1H), 7.60 (d, J = 8.6 Hz, 1H), 7.39-7.26 (m, 4H), 3.51-3.46 (m, 2H), 2.96 (t, J = 7.1 Hz, 2H), 2.76 (d, J = 4.4 Hz, 3H). | F |
| 111 | 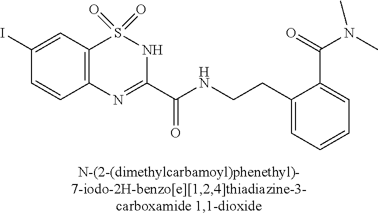 [SMILES: CN(C)C(=O)c1ccccc1CCNC(=O)C1=Nc2ccc(I)cc2S(=O)(=O)N1] | LCMS (ES- API): Rₜ 2.55 min, m/z 526.7 [M + H]⁺ | ¹H NMR (400 MHz, d₆-DMSO) δ 12.7 (brs, 1H), 9.28 (t, J = 5.5 Hz, 1H), 8.08 (d, J = 1.6 Hz, 1H), 8.04 (dd, J = 8.8, 1.6 Hz, 1H), 7.59 (d, J = 8.8 Hz, 1H), 7.38-7.30 (m, 2H), 7.29-7.24 (m, 1H), 7.16 (d, J = 7.4 Hz, 1H), 3.49- 3.42 (m, 2H), 3.01 (s, 3H), 2.80 | F |
| | | | (t, J = 6.8 Hz, 2H), | |
| | | | 2.77 (s, 3H). | |

| Example | Structure | LCMS | Method |
|---|---|---|---|
| 112 | 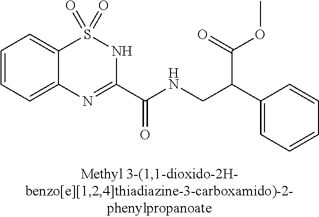 [SMILES: COC(=O)C(CNC(=O)C1=Nc2ccccc2S(=O)(=O)N1)c1ccccc1] | LCMS: r.t. 3.520 min; m/z 388.2 M + H⁺. | G-1 |
| 113 | 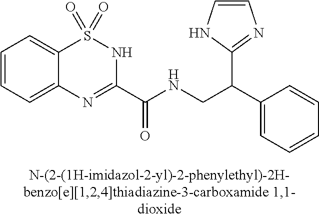 [SMILES: O=C(NCC(c1ccccc1)c1ncc[nH]1)C1=Nc2ccccc2S(=O)(=O)N1] | LCMS: r.t. 4.503 min; m/z = 396.2 [M + H]⁺. | G-2 |
| 114 | 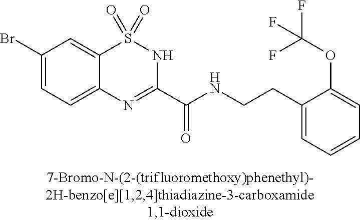 [SMILES: O=C(NCCc1ccccc1OC(F)(F)F)C1=Nc2ccc(Br)cc2S(=O)(=O)N1] | LCMS-B: rt 3.51 min; m/z 489.8 [M − H]⁻ | B-3 |
| 115 | 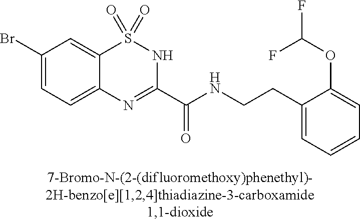 [SMILES: O=C(NCCc1ccccc1OC(F)F)C1=Nc2ccc(Br)cc2S(=O)(=O)N1] | LCMS-B: rt 3.44 min; m/z 473.7 [M − H]⁻ | B-4 |
| 116 | 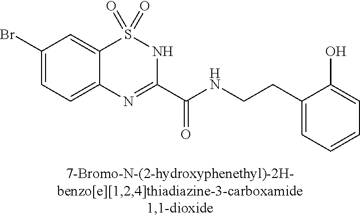 [SMILES: O=C(NCCc1ccccc1O)C1=Nc2ccc(Br)cc2S(=O)(=O)N1] | LCMS-B: rt 3.29 min; m/z 421.7 [M − H]⁻ | B-4 |
| 117 | 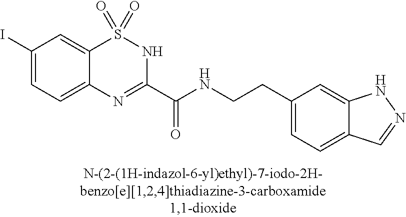 [SMILES: O=C(NCCc1ccc2cn[nH]c2c1)C1=Nc2ccc(I)cc2S(=O)(=O)N1] | LCMS-B rt 3.36 min; 495.7 [M + H]⁺ | B-1 |
| 118 | 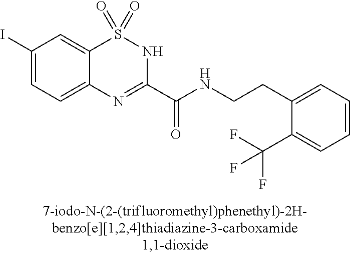 [SMILES: O=C(NCCc1ccccc1C(F)(F)F)C1=Nc2ccc(I)cc2S(=O)(=O)N1] | LCMS-B rt 3.70 min; m/z 521.7 [M − H]⁻ | A-1 |
| 119 | 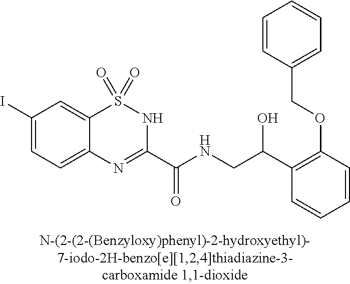 [SMILES: O=C(NCC(O)c1ccccc1OCc1ccccc1)C1=Nc2ccc(I)cc2S(=O)(=O)N1] | LCMS-B: rt 3.46 min; m/z 575.7 [M − H]⁻ | B-1 |

| Example | Structure | LCMS | Method |
|---|---|---|---|
| 120 | 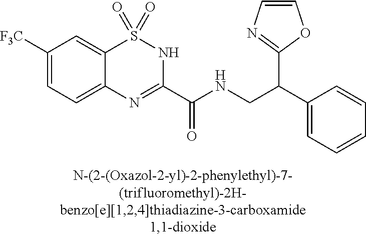 [SMILES: O=C(NCC(c1ccccc1)c1ncco1)C1=Nc2ccc(C(F)(F)F)cc2S(=O)(=O)N1] | LCMS-B rt 3.35 min; m/z 464.7 [M + H]⁺ | A-1 |
| 121 | 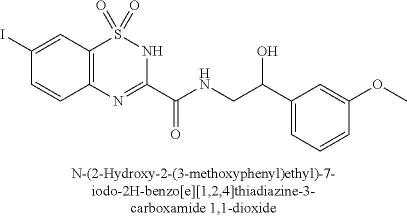 [SMILES: COc1cccc(C(O)CNC(=O)C2=Nc3ccc(I)cc3S(=O)(=O)N2)c1] | LCMS-B rt 3.21 min; m/z 499.7 [M − H]⁻ | B-1 |
| 122 | 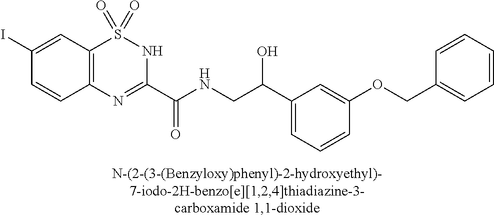 [SMILES: O=C(NCC(O)c1cccc(OCc2ccccc2)c1)C1=Nc2ccc(I)cc2S(=O)(=O)N1] | LCMS-B rt 3.40 min; m/z 575.7 [M − H]⁻ | A-1 |
| 123 | 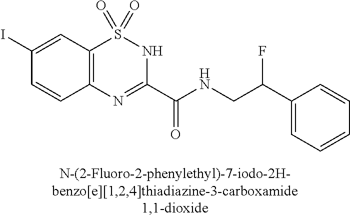 [SMILES: O=C(NCC(F)c1ccccc1)C1=Nc2ccc(I)cc2S(=O)(=O)N1] | LCMS-B rt 3.36 min; m/z 471.7 [M − H]⁻ | B-1 |
| 124 | 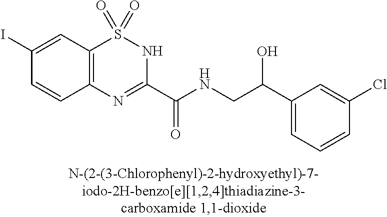 [SMILES: O=C(NCC(O)c1cccc(Cl)c1)C1=Nc2ccc(I)cc2S(=O)(=O)N1] | LCMS-B rt 3.30 min; m/z 503.7 [M − H]⁻ | B-1 |
| 125 | 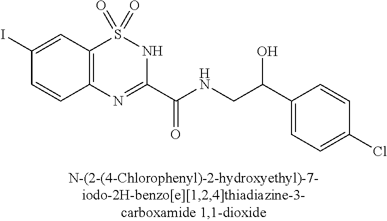 [SMILES: O=C(NCC(O)c1ccc(Cl)cc1)C1=Nc2ccc(I)cc2S(=O)(=O)N1] | LCMS-B rt 3.31 min; m/z 503.7 [M − H]⁻ | A-1 |
| 126 | 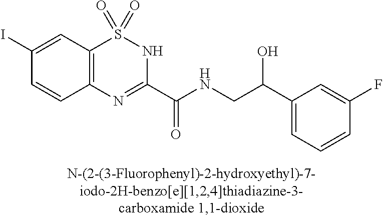 [SMILES: O=C(NCC(O)c1cccc(F)c1)C1=Nc2ccc(I)cc2S(=O)(=O)N1] | LCMS-B rt 3.25 min; m/z 487.7 [M − H]⁻ | A-1 |
| 127 | 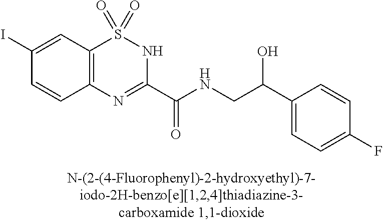 [SMILES: O=C(NCC(O)c1ccc(F)cc1)C1=Nc2ccc(I)cc2S(=O)(=O)N1] | LCMS-B rt 3.24 min; m/z 487.7 [M − H]⁻ | A-1 |
| 128 | 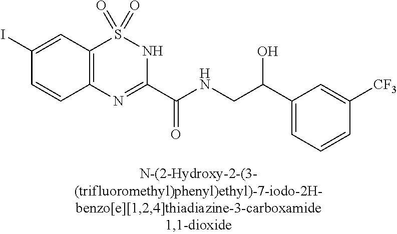 [SMILES: O=C(NCC(O)c1cccc(C(F)(F)F)c1)C1=Nc2ccc(I)cc2S(=O)(=O)N1] | LCMS-B rt 3.35 min; m/z 537.7 [M − H]⁻ | B-1 |

Method H:

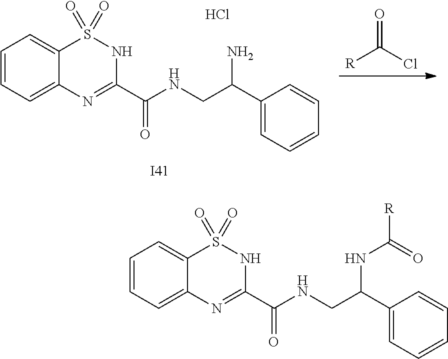

To a mixture of N-(2-amino-2-phenylethyl)-2H-benzo[e][1,2,4]thiadiazine-3-carboxamide 1,1-dioxide hydrochloride (I41) (0.18 mmol) in DCM (3 mL) was added TEA (3 eq) and the acyl chloride (1.2 eq). The mixture was stirred at r.t. for 3 h under N₂ atmosphere. The mixture was diluted with DCM and washed with water (×2), 1 M HCl, brine, dried over Na₂SO₄ and concentrated to give the crude product which was purified by preparative TLC (DCM/MeOH=20:1) to give the desired product.

Method I:

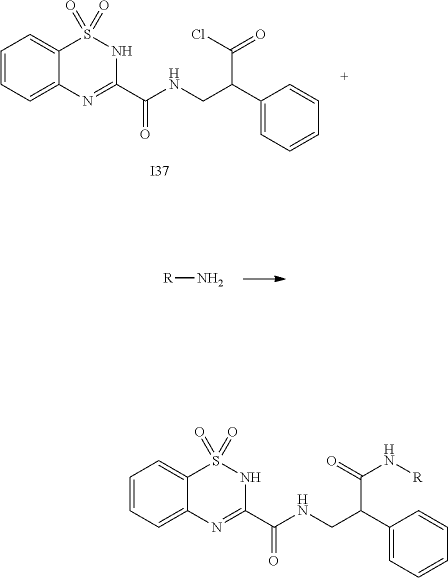

A solution of 3-(1,1-dioxido-2H-benzo[e][1,2,4]thiadiazine-3-carboxamido)-2-phenylpropanoyl chloride (I37) (0.13 mmol) and TEA (10 eq) in DCM (5 mL) was stirred at 0° C. under N₂ for 10 min. The amine (5 eq) was then added and the mixture was stirred at r.t. for 30 min. Water and 1 M HCl were added and the mixture was extracted with DCM. The organic layer was dried over sodium sulfate, concentrated and the residue was purified by preparative TLC (DCM/MeOH=20:1) to afford the desired product.

Method J:

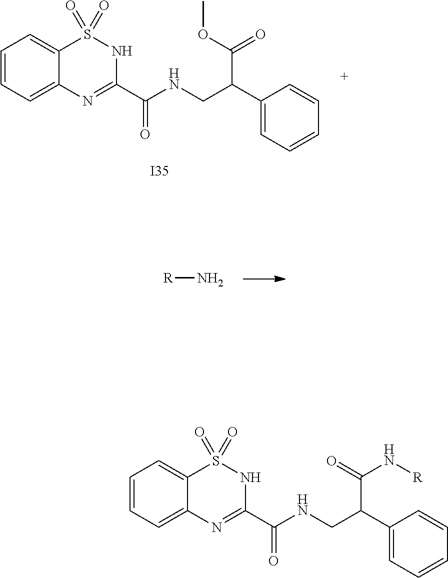

Methyl 3-(1,1-dioxido-2H-benzo[e][1,2,4]thiadiazine-3-carboxamido)-2-phenylpropanoate (I35; 112) (0.18 mmol) was dissolved in the appropriate amine solution (5 mL) and the mixture was heated at 120° C. for 90 min in the microwave. The solvent was removed and the residue was purified by preparative TLC (DCM/MeOH=20:1) to afford the desired product.

Method K:

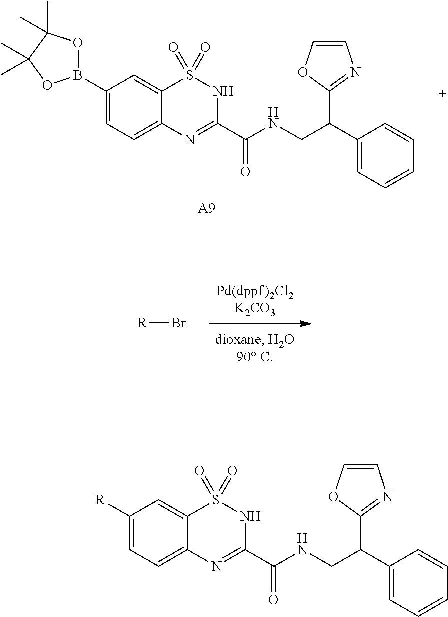

A mixture of N-(2-(oxazol-2-yl)-2-phenylethyl)-7-(4,4,5,5-tetramethyl-1,3,2-dioxaborolan-2-yl)-2H-benzo[e][1,2,4]thiadiazine-3-carboxamide 1,1-dioxide (A9) (0.1 mmol), R—Br (4 eq), Pd(dppf)₂Cl₂ (0.1 eq), K₂CO₃ (4 eq) in dioxane (3 mL) and water (0.5 mL) was stirred under N₂ at 90° C. for 3 h. The mixture was then allowed to cool to r.t. and extracted with EtOAc. The combined organic extracts were washed with brine, dried over sodium sulfate and concentrated to give a residue which was purified by preparative TLC (DCM/MeOH=20:1) to give the desired product.

| Example | Name and structure | LCMS data | ¹H NMR data | Method |
|---|---|---|---|---|
| 129 | 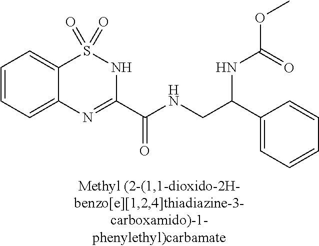 [SMILES: COC(=O)NC(CNC(=O)C1=Nc2ccccc2S(=O)(=O)N1)c1ccccc1] | LCMS (ES- API): Rₜ 2.18 min, m/z 403.1 [M + H]⁺ | ¹H NMR (400 MHz, d₆-DMSO) δ 12.6 (s, 1H), 9.19 (m, 1H), 7.87-7.71 (m, 4H), 7.55-7.51 (m, 1H), 7.34-7.33 (m, 4H), 7.27-7.24 (m, 1H), 4.92-4.83 (m, 1H), 3.59-3.54 (m, 1H), 3.50 (s, 3H), 3.31- 3.29 (m, 1H). | H |
| 130 | 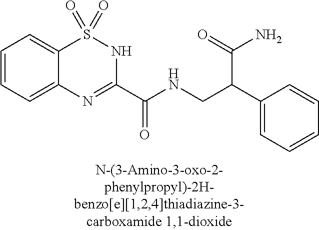 [SMILES: NC(=O)C(CNC(=O)C1=Nc2ccccc2S(=O)(=O)N1)c1ccccc1] | LCMS (ES- API): Rₜ 1.91 min, m/z 373.1 [M + H]⁺ | ¹H NMR (400 MHz, d₆-DMSO) δ 12.6 (s, 1H), 9.06 (t, J = 6.0 Hz, 1H), 7.85-7.79 (m, 2H), 7.75-7.71 (m, 1H), 7.54-7.50 (m, 2H), 7.36-7.29 (m, 4H), 7.26-7.22 (m, 1H), 6.98 (s, 1H), 3.91 (t, J = 7.3 Hz, 1H), 3.79-3.71 (m, 1H), 3.67-3.60 (m, 1H). | I |
| 131 | 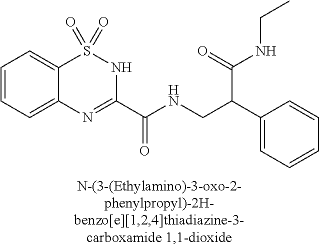 [SMILES: CCNC(=O)C(CNC(=O)C1=Nc2ccccc2S(=O)(=O)N1)c1ccccc1] | LCMS (ES- API): Rₜ 1.03 min, m/z 401.1 [M + H]⁺ | ¹H NMR (400 MHz, CDCl₃) δ 10.0 (s, 1H), 8.23 (t, J = 6.0 Hz, 1H), 7.97 (d, J = 8.0 Hz, 1H), 7.61 (m, 1H), 7.48 (m, 1H), 7.39-7.33 (m, 3H), 7.30-7.28 (m, 3H), 5.37 (m, 1H), 4.00- 3.93 (m, 1H), 3.87- 3.80 (m, 1H), 3.68- 3.64 (m, 1H), 3.32- 3.24 (m, 2H), 1.06 (t, J = 7.3 Hz, 3H). | J |
| 132 | 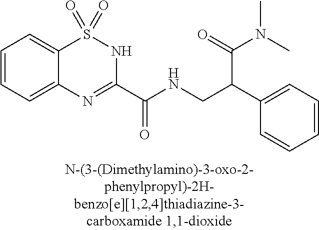 [SMILES: CN(C)C(=O)C(CNC(=O)C1=Nc2ccccc2S(=O)(=O)N1)c1ccccc1] | LCMS (ES- API): Rₜ 2.29 min, m/z 401.1 [M + H]⁺ | ¹H NMR (400 MHz, CDCl₃) δ 10.0 (s, 1H), 8.27 (t, J = 6.0 Hz, 1H), 7.98 (d, J = 7.6 Hz, 1H), 7.61- 7.57 (m, 1H), 7.48- 7.44 (m, 1H), 7.37- 7.28 (m, 6H), 4.02- 3.99 (m, 1H), 3.92- 3.85 (m, 1H), 3.83- 3.76 (m, 1H), 2.98 (s, 3H), 2.79 (s, 3H). | I |

Chiral Separation

Some of the racemates produced above were separated using chiral columns as described below

| | | | | LCMS |
|---|---|---|---|---|
| Racemate | Enantiomer | SFC Purification Method | SFC data | data* |
| 46 | Enantiomer 1 - | Instrument: Waters SFC-80; Column: | SFC: rt. | rt. 2.749 min; |
| | 136 | Lux C3 (250*30)mm, 5μ | 2.41 min | m/z 397.2 [M + H]+ |
| | Enantiomer 2 - | Mobile Phase: CO2: MeOH (70:30); | SFC: rt | rt. 2.744 min; |
| | 137 | Total flow: 60 ml/min | 4.04 min | m/z 397.2 [M + H]+ |
| | | Back Pressure: 100 bar; Wave length: | | |
| | | 210 nm; Cycle time: 10 min | | |
| 1 | Enantiomer 1 - | Instrument: Waters SFC-80; Column: | SFC: rt | rt. 3.045 min; |
| | 138 | Lux C3 (250*30)mm, 5μ | 3.83 min | m/z 475.0 [M + H]+ |
| | Enantiomer 2 - | Mobile Phase: CO2: MeOH (70:30); | SFC: rt | rt. 3.044 min; |
| | 139 | Total flow: 60 ml/min | 5.64 min | m/z 475.0 [M + H]+ |
| | | Back Pressure: 100 bar; Wave length: | | |
| | | 210 nm; Cycle time: 10 min | | |
| 48 | Enantiomer 1 - | Instrument: Waters SFC-80; Column: | SFC: rt | rt. 2.638 min; |
| | 140 | YMC Amylose C (250*30)mm, 5μ | 3.19 min | m/z 412.2 [M + H]+ |
| | Enantiomer 2 - | Mobile Phase: CO2: MeOH (60:40); | SFC: rt | rt. 2.6220 min; |
| | 141 | Total flow: 60 ml/min | 4.02 min | m/z 412.2 [M + H]+ |
| | | Back Pressure: 100 bar; Wave length: | | |
| | | 210 nm; Cycle time: 10 min | | |
| 4 | Enantiomer 1 - | Instrument: Waters SFC-80; Column: | SFC: rt | n/a |
| | 142 | Chiralpak ADH (250*20)mm, 5μ | 3.88 min | |
| | Enantiomer 2 - | Mobile Phase: CO2: MeOH (60:40); | SFC: rt | n/a |
| | 143 | Total flow: 40 mL/min | 5.91 min | |
| | | Back Pressure: 100 bar; Wave length: | | |
| | | 210 nm; Cycle time: 7 min | | |
| 36 | Enantiomer 1 - | Instrument: Waters SFC-80; Column: | SFC: rt | n/a |
| | 144 | Chiralpak ADH (250*20)mm, 5μ | 4.76 min | |
| | Enantiomer 2 - | Mobile Phase: CO2: MeOH (60:40); | SFC: rt | n/a |
| | 145 | Total flow: 40 mL/min | 6.17 min | |
| | | Back Pressure: 100 bar; Wave length: | | |
| | | 210 nm; Cycle time: 7 min | | |
| 41 | Enantiomer 1 - | Instrument: Waters SFC-80; Column: | SFC: rt | n/a |
| | 146 | Lux C3 (250*20)mm, 5μ | 2.22 min | |
| | Enantiomer 2 - | Mobile Phase: CO2: MeOH (60:40); | SFC: rt | n/a |
| | 147 | Total flow: 60 mL/min; | 3.62 min | |
| | | Back Pressure: 100 bar; Wave length: | | |
| | | 304 nm; Cycle time: 6 min | | |
| 8 | Enantiomer 1 - | Instrument: Waters SFC-80; Column: | SFC: rt | n/a |
| | 148 | Lux A1 (250*30)mm, 5μ | 5.17 min | |
| | Enantiomer 2 - | Mobile Phase: CO2: IPA (60:40); | SFC: rt | n/a |
| | 149 | Total flow: 60 mL/min | 6.84 min | |
| | | Back Pressure: 100 bar; Wave length: | | |
| | | 312 nm; Cycle time: 5 min | | |
| 94 | Enantaiomer 1 - | Instrument: Waters SFC-80; Column: | SFC: rt | n/a |
| | 150 | YMC Cellulose-SC (250*30)mm, 5μ | 3.58 min | |
| | | Mobile Phase: CO2: MeOH (60:40); | | |
| | | Total flow: 60 mL/min | | |
| | | Back Pressure: 100 bar; Wave length: | | |
| | | 304 nm; Cycle time: 6 min | | |
| 113 | Enantiomer 1 - | ChiralPak IA, 250 × 4.6 mm with 1:1 | rt 15.6 min | n/a |
| | 151 | EtOH: hexane mobile phase. | | |
| | Enantiomer 2 - | | rt 20.5 min | n/a |
| | 152 | | | |

*LC-MS details: Column: ZORBAX Extend C18 (50 × 4.6 mm 5μ); MOBILE PHASE: A: 0.1% HCOOH IN WATER, B: METHANOL; FLOW RATE : 1.5 mL/min

### Example 153: 7-(Methylsulfonamido)-N-(2-(oxazol-2-yl)-2-phenylethyl)-2H-benzo[e][1,2,4]thiadiazine-3-carboxamide 1,1-dioxide (I53)

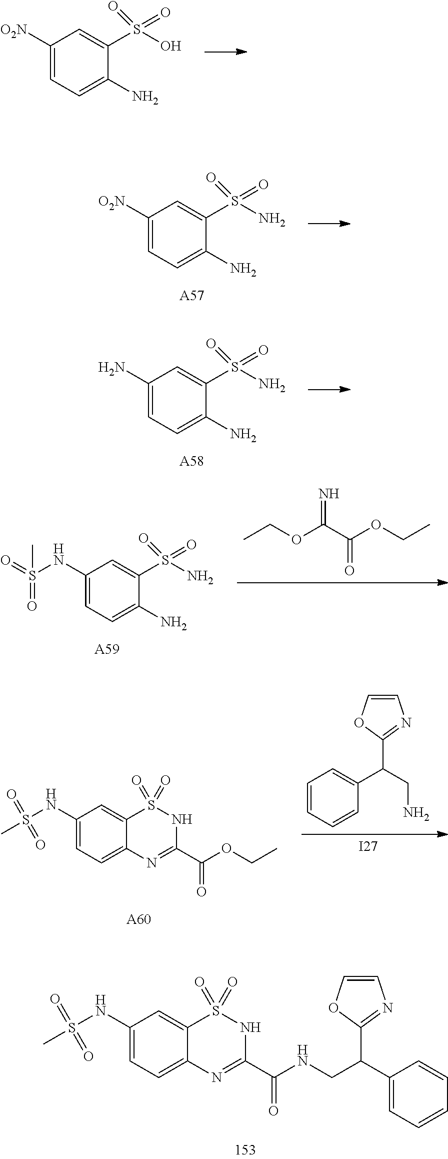

### a) 2-Amino-5-nitrobenzenesulfonamide (A57)

POCl₃(6.86 mL, 82.2 mmol) was slowly added to a mixture of 2-amino-5-nitrobenzenesulfonic acid (3.00 g, 27.4 mmol) in sulfolane (20 mL) at r.t. and the mixture was heated at 120° C. for 3.5 h. The mixture was allowed to cool to r.t. then slowly poured into conc. NH₄OH (60 mL). The resulting precipitate was collected by filtration, washed with water (100 mL) and dried to give the product (1.90 g, 31% yield) as a yellow solid. LCMS (ES-API): Rₜ 0.43 min; m/z 218.1 [M+H]⁺.

### b) 2,5-Diaminobenzenesulfonamide (A58)

To a solution of 2-amino-5-nitrobenzenesulfonamide (A57) (1.9 g, 8.7 mmol) in MeOH (20 mL) was added 10% Pd/C (190 mg) and the mixture was stirred at r.t. under H₂ (1 atm) for 16 h. The mixture was filtered and the filtrate was concentrated to give the product as a brown solid (1.3 g, 79% yield). LCMS (ES-API): Rₜ 0.342 min; m/z 188.1 [M+H]⁺.

### c) 2-Amino-5-(methylsulfonamido)benzenesulfonamide (A59)

To a solution of 2,5-diaminobenzenesulfonamide (A58) (1.3 g, 0.69 mmol) in acetonitrile (20 mL) at r.t. was added pyridine (79 mg, 1.03 mmol) and MsCl (795 mg, 0.69 mmol) and the mixture was stirred at r.t. for 15 h. Diethyl ether (10 mL) was added and the resulting precipitate was collected by filtration and washed with diethyl ether (30 mL) to give the product as a yellow solid (1.4 g, 90% yield). LCMS (ES-API): Rₜ 2.53 min; m/z 266.1 [M+H]⁺.

### d) Ethyl 7-(methylsulfonamido)-2H-benzo[e][1,2,4]thiadiazine-3-carboxylate 1,1-dioxide (A60)

To a solution of 2-amino-5-(methylsulfonamido)benzenesulfonamide (A59) (1.3 g, 4.9 mmol) in EtOH (20 mL) was added ethyl 2-ethoxy-2-iminoacetate (1.42 g, 9.8 mmol) and the mixture was heated at 100° C. for 15 h. After cooling to r.t., the precipitate was collected by filtration and washed with diethyl ether (20 mL) to give the product as a white solid (1.2 g, 70% yield). LCMS (ES-API): Rₜ 0.584 min; m/z 347.8 [M+H]⁺.

### e) 7-(Methylsulfonamido)-N-(2-(oxazol-2-yl)-2-phenylethyl)-2H-benzo[e][1,2,4]thiadiazine-3-carboxamide 1,1-dioxide (153)

To a solution of ethyl 7-(methylsulfonamido)-2H-benzo[e][1,2,4]thiadiazine-3-carboxylate 1,1-dioxide (A60) (85 mg, 0.24 mmol) in EtOH (3 mL) was added 2-(oxazol-2-yl)-2-phenylethanamine (I27) (51 mg, 0.27 mmol) and the mixture was heated at 100° C. for 15 h then allowed to cool to r.t. The solvent was removed under reduced pressure and the residue was diluted with water (5 mL) and extracted with EtOAc (8 mL×3). The combined organic extracts were dried over Na₂SO₄ and concentrated. The residue was purified by prep. TLC (DCM/MeOH=10:1) to give the product as a white solid (20 mg, 17% yield). ¹H NMR (400 MHz, d₆-DMSO) δ 12.7 (brs, 1H), 10.2 (brs, 1H), 9.26 (t, J=5.8 Hz, 1H), 8.04 (d, J=0.4 Hz, 1H), 7.78 (d, J=8.7 Hz, 1H), 7.58-7.52 (m, 2H), 7.36-7.31 (m, 2H), 7.29-7.26 (m, 3H), 7.20 (d, J=0.4 Hz, 1H), 4.67 (t, J=7.6 Hz, 1H), 4.03-3.95 (m, 1H), 3.92-3.84 (m, 1H), 3.05 (s, 3H). LCMS (ES-API): Rₜ 2.31 min; m/z 489.8 [M+H]⁺.

General Method L

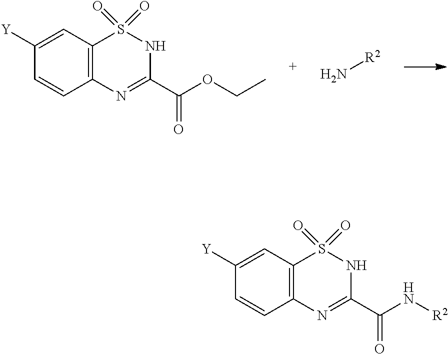

To a solution of the ester (x mmol) and amine (x mmol) in EtOH (x mL) was added Et₃N (3 equivalents) and the mixture was heated at 110° C. in a sealed tube overnight. The mixture was concentrated under reduced pressure and the residue was purified by silica gel chromatography (DCM/MeOH=20/1) to give the title compound.

General Method M

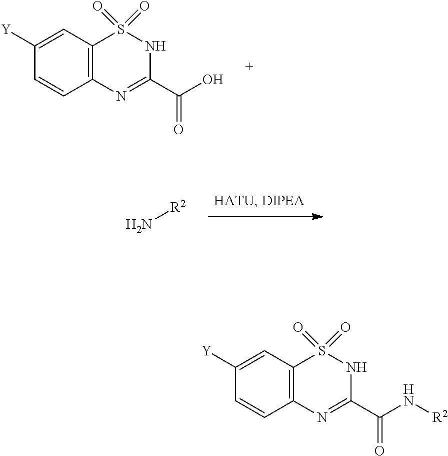

To a solution of the acid (x mmol), HATU (x mmol) and DIPEA (x mmol) in DMF (x mL) or MeCN (x mL) was added the amine (x mmol) and the mixture was stirred at RT overnight. Water was added and the mixture was extracted with EtOAc. The combined organic extracts were dried over Na₂SO₄, filtered and concentrated under reduced pressure. The residue was purified by silica gel chromatography (DCM/MeOH=20/1) to give the title compound.

General Method N

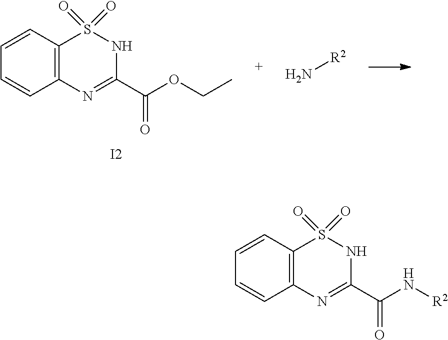

To a suspension of ethyl 4H-benzo[e][1,2,4]thiadiazine-3-carboxylate 1,1-dioxide 12 (x mmol) in EtOH (0.125 mL) was added the amine (x mmol) and for some examples triethylamine (x mmol). The mixture was subjected to microwave irradiation at 100° C. for 30 min. Method for isolation of product specified in Table L.

General Method 0

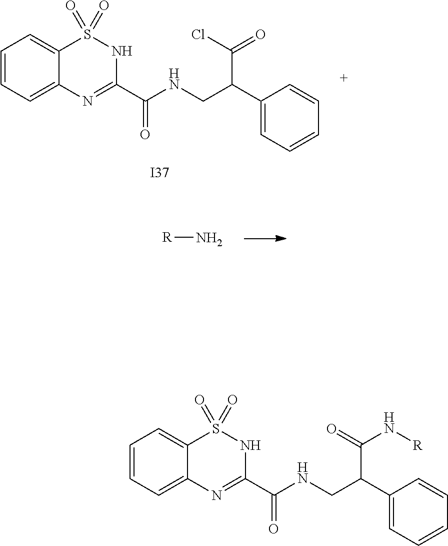

A solution of 3-(1,1-dioxido-2H-benzo[e][1,2,4]thiadiazine-3-carboxamido)-2-phenylpropanoyl chloride (I37) (0.13 mmol) and TEA (10 eq) in DCM (5 mL) was stirred at 0° C. under N₂ for 10 min. The amine (5 eq) was then added and the mixture was stirred at room temperature. for 30 min. Water and 1 M HCl were added and the mixture was extracted with DCM. The organic layer was dried over sodium sulfate, concentrated and the residue was purified by preparative TLC (DCM/MeOH=20:1) to afford the desired product.

General Method P

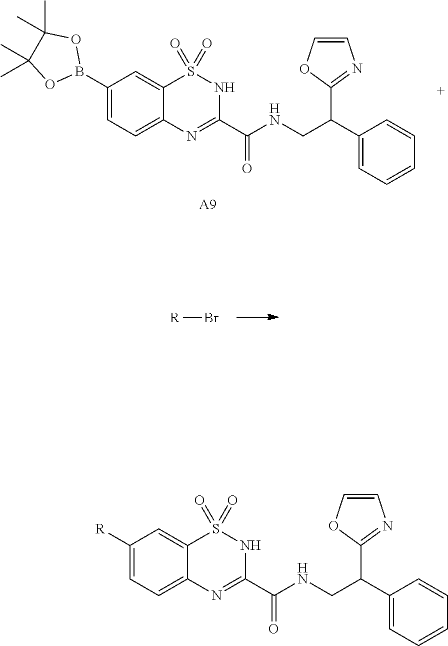

A mixture of N-(2-(oxazol-2-yl)-2-phenylethyl)-7-(4,4,5,5-tetramethyl-1,3,2-dioxaborolan-2-yl)-2H-benzo[e][1,2,4]thiadiazine-3-carboxamide 1,1-dioxide (A9) (0.1 mmol), R—Br (4 eq), Pd(dppf)₂Cl₂ (0.1 eq), K₂CO₃ (4 eq) in 1,4-dioxane (3 mL) and water (0.5 mL) was stirred under N₂ at 90° C. for 3 h. The mixture was then allowed to cool to room temperature and extracted with EtOAc. The combined organic extracts were washed with brine, dried over sodium sulfate and concentrated to give a residue which was purified by preparative TLC (DCM/MeOH=20:1) to give the desired product.

The following examples were prepared according to the procedures described in general methods L-P using the specified quantities of reagents.

TABLE L

| Example | Name and structure | Analytical data | Method | Notes |
|---|---|---|---|---|
| 157 | 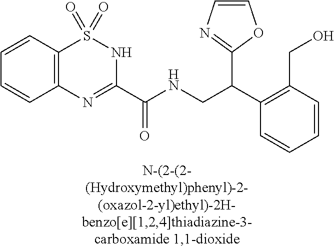 [SMILES: O=C(NCC(c1ncco1)c1ccccc1CO)C1=Nc2ccccc2S(=O)(=O)N1] | LCMS-C: Rₜ 0.78 min, m/z 427.0 [M + H]⁺; ¹H NMR (400 MHz, DMSO-d₆) δ 12.4 (br s, 1H), 9.30 (t, J = 5.6 Hz, 1H), 8.01 (s, 1H), 7.85-7.79 (m, 2H), 7.74 (t, J = 8.8 Hz, 1H), 7.53 (t, J = 8.4 Hz, 1H), 7.45-7.43 (m, 1H), 7.28-7.18 (m, 4H), 5.27 (t, J = 5.6 Hz, 1H), 4.96- 4.94 (m, 1H), 4.70 (d, J = 5.2 Hz, 2H), 4.10-4.05 (m, 1H), 3.80-3.74 (m, 1H). | L | Ester l2 (0.18 mmol), amine l110 (0.15 mmol) EtOH (2 mL) |
| 158 | 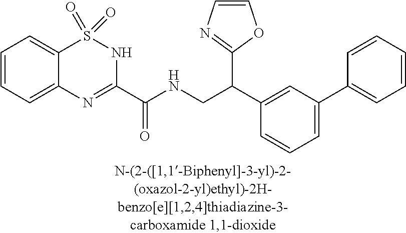 [SMILES: O=C(NCC(c1cccc(-c2ccccc2)c1)c1ncco1)C1=Nc2ccccc2S(=O)(=O)N1] | LCMS-C: Rₜ 2.26 min, m/z 473.0 [M + H]⁺; ¹H NMR (400 MHz, DMSO-d₆) δ 9.16 (br s, 1H), 8.07 (s, 1H), 7.76 (br s, 1H), 7.59-7.42 (m, 6H), 7.41-7.23 (m, 8H), 4.74 (t, J = 7.2 Hz, 1H), 4.01-3.94 (m, 2H). | L | Ester l2 (0.12 mmol), amine 1l51 (0.12 mmol) EtOH (2 mL) Heated at 120° C. overnight |
| 159 | 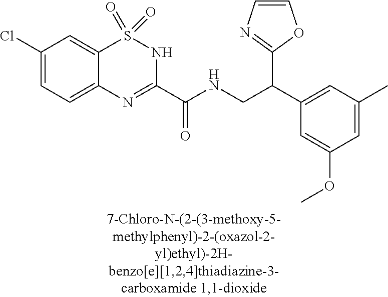 [SMILES: COc1cc(C)cc(C(CNC(=O)C2=Nc3ccc(Cl)cc3S(=O)(=O)N2)c2ncco2)c1] | LCMS-C: Rₜ 2.31 min, m/z 474.9 [M + H]⁺; ¹H NMR (400 MHz, DMSO-d₆) δ 12.8 (br s, 1H), 8.94 (br s, 1H), 8.04 (s, 1H), 7.74 (s, 1H), 7.66 (d, J = 8.8 Hz, 1H), 7.57 (d, J = 8.8 Hz, 1H), 7.20 (s, 1H), 6.65-6.61 (m, 3H), 4.56 (t, J = 7.6 Hz, 1H), 3.96-3.78 (m, 2H), 3.69 (s, 3H), 2.23 (s, 3H). | L | Ester l162 (0.43 mmol), amine l150 (0.52 mmol) EtOH (3 mL) Reaction mixture was diluted with water and extracted with EtOAc. Organic extract was dried over Na₂SO₄ and concentrated to give the title compound. |
| 160 | 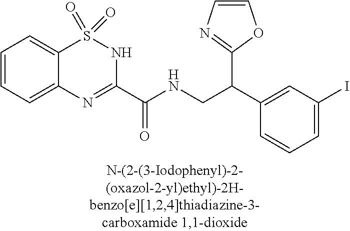 [SMILES: O=C(NCC(c1cccc(I)c1)c1ncco1)C1=Nc2ccccc2S(=O)(=O)N1] | LCMS-C: Rₜ 2.14 min, m/z 522.9 [M + H]⁺; ¹H NMR (400 MHz, DMSO-d₆) δ 12.6 (br s, 1H), 9.33 (t, J = 5.6 Hz, 1H), 8.08 (s, 1H), 7.86-7.80 (m, 2H), 7.75 (t, J = 8.0 Hz, 1H), 7.66-7.64 (m, 2H), 7.55 (t, J = 7.6 Hz, 1H), 7.31 (d, J = 7.6 Hz, 1H), 7.23 (s, 1H), 7.17 (t, J = 7.6 Hz, 1H), 4.67 (t, J = 7.6 Hz, 1H), 4.01-3.96 (m, 1H), 3.89-3.82 (m, 1H). | L | Ester l2 (1.0 mmol), amine l162 (1.0 mmol) EtOH (8 mL) Heated at 120° C. for 3 h |
| 161 | 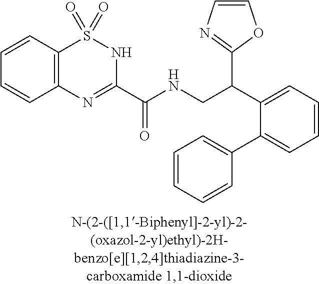 [SMILES: O=C(NCC(c1ncco1)c1ccccc1-c1ccccc1)C1=Nc2ccccc2S(=O)(=O)N1] | LCMS-C: Rₜ 2.21 min, m/z 473.0 [M + H]⁺; ¹H NMR (400 MHz, DMSO-d₆) δ 12.5 (br s, 1H), 9.17 (t, J = 5.6 Hz, 1H), 8.03 (s, 1H), 7.86 (d, J = 7.6 Hz, 1H), 7.82 (d, J = 8.0 Hz, 1H), 7.75 (t, J = 7.6 Hz, 1H), 7.55 (t, J = 7.6 Hz, 1H), 7.39- 7.29 (m, 8H), 7.21-7.18 (m, 2H), 4.74 (t, J = 7.2 Hz, 1H), 4.05-3.97 (m, 1H), 3.71-3.64 (m, 1H). | L | Ester l2 (0.12 mmol), amine l136 (0.10 mmol) EtOH (2 mL) |
| 162 | 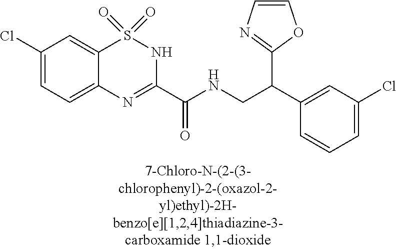 [SMILES: O=C(NCC(c1cccc(Cl)c1)c1ncco1)C1=Nc2ccc(Cl)cc2S(=O)(=O)N1] | LCMS-C: Rₜ 2.22 min, m/z 464.9 [M + H]⁺; ¹H NMR (400 MHz, DMSO-d₆) δ 12.8 (br s, 1H), 9.33 (t, J = 5.6 Hz, 1H), 8.08 (s, 1H), 7.89 (s, 1H), 7.84-7.79 (m, 2H), 7.38-7.33 (m, 3H), 7.25-7.22 (m, 2H), 4.70 (t, J = 7.2 Hz, 1H), 4.05- 3.85 (m, 2H). | L | Ester l162 (0.28 mmol), amine l128 (0.33 mmol) EtOH (5 mL) |
| 163 | 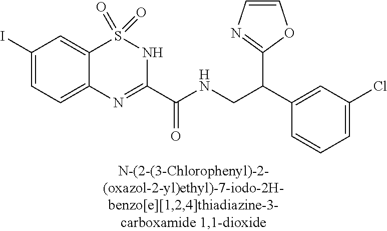 [SMILES: O=C(NCC(c1cccc(Cl)c1)c1ncco1)C1=Nc2ccc(I)cc2S(=O)(=O)N1] | LCMS-C: Rₜ 2.23 min, m/z 556.8 [M + H]⁺; ¹H NMR (400 MHz, DMSO-d₆) δ 12.7 (br s, 1H), 9.14 (br s, 1H), 8.07 (s, 1H), 8.00 (s, 1H), 7.97 (d, J = 8.4 Hz, 1H), 7.48 (d, J = 8.8 Hz, 1H), 7.38-7.33 (m, 3H), 7.24-7.22 (m, 2H), 4.70 (t, J = 7.6 Hz, 1H), 4.01-3.92 (m, 1H), 3.90-3.83 (m, 1H). | L | Ester l7 (0.21 mmol), amine l128 (0.24 mmol) EtOH (5 mL) |
| 164 | 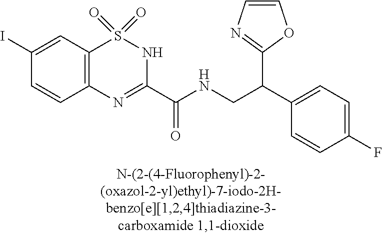 [SMILES: O=C(NCC(c1ccc(F)cc1)c1ncco1)C1=Nc2ccc(I)cc2S(=O)(=O)N1] | LCMS-C: Rₜ 2.20 min, m/z 540.8 [M + H]⁺; ¹H NMR (400 MHz, DMSO-d₆) δ 12.7 (br s, 1H), 9.16 (br s, 1H), 8.06-7.98 (m, 3H), 7.51 (d, J = 8.4 Hz, 1H), 7.32-7.29 (m, 2H), 7.20-7.13 (m, 3H), 4.68 (t, J = 7.6 Hz, 1H), 3.98-3.91 (m, 1H), 3.87-3.80 (m, 1H). | L | Ester l7 (0.21 mmol), amine l124 (0.25 mmol) EtOH (5 mL) |
| 165 | 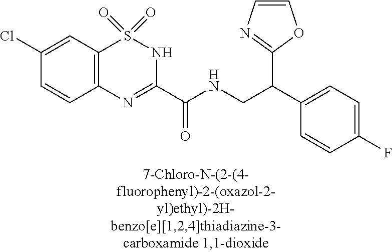 [SMILES: O=C(NCC(c1ccc(F)cc1)c1ncco1)C1=Nc2ccc(Cl)cc2S(=O)(=O)N1] | LCMS-C: Rₜ 2.13 min, m/z 448.9 [M + H]⁺; ¹H NMR (400 MHz, DMSO-d₆) δ 12.8 (br s, 1H), 9.13 (br s, 1H), 8.05 (s, 1H), 7.81 (s, 1H), 7.71 (s, 2H), 7.33-7.29 (m, 2H), 7.20-7.13 (m, 3H), 4.68 (t, J = 7.6 Hz, 1H), 3.98-3.92 (m, 1H), 3.87-3.81 (m, 1H). | L | Ester l162 (0.28 mmol), amine l124 (0.33 mmol) EtOH (5 mL) |
| 166 | 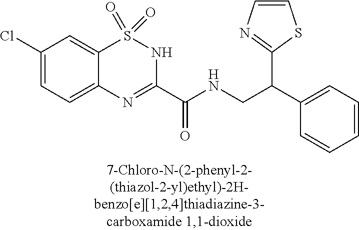 [SMILES: O=C(NCC(c1ccccc1)c1nccs1)C1=Nc2ccc(Cl)cc2S(=O)(=O)N1] | LCMS-C: Rₜ 2.19 min, m/z 446.9 [M + H]⁺; ¹H NMR (400 MHz, DMSO-d₆) δ 12.8 (br s, 1H), 9.32 (t, J = 6.0 Hz, 1H), 7.91 (d, J = 1.6 Hz, 1H), 7.84-7.81 (m, 2H), 7.79 (d, J = 3.2 Hz, 1H), 7.63 (d, J = 3.2 Hz, 1H), 7.39-7.25 (m, 5H), 4.90 (t, J = 7.6 Hz, 1H), 4.11-4.04 (m, 1H), 4.02-3.95 (m, 1H). | L | Ester l162 (0.10 mmol), amine l113 (0.10 mmol) EtOH (2.5 mL) |
| 167 | 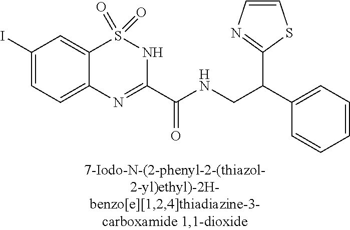 [SMILES: O=C(NCC(c1ccccc1)c1nccs1)C1=Nc2ccc(I)cc2S(=O)(=O)N1] | LCMS-C: Rₜ 2.23 min, m/z 538.8 [M + H]⁺; ¹H NMR (400 MHz, DMSO-d₆) δ 12.7 (br s, 1H), 9.30 (t, J = 6.0 Hz, 1H), 8.08-8.04 (m, 2H), 7.79 (d, J = 3.2 Hz, 1H), 7.63 (d, J = 3.2 Hz, 1H), 7.59 (d, J = 8.8 Hz, 1H), 7.38-7.25 (m, 5H), 4.89 (t, J = 7.6 Hz, 1H), 4.10-4.03 (m, 1H), 4.01-3.94 (m, 1H). | L | Ester l7 (0.10 mmol), amine l113 (0.10 mmol) EtOH (2.5 mL) |
| 168 | 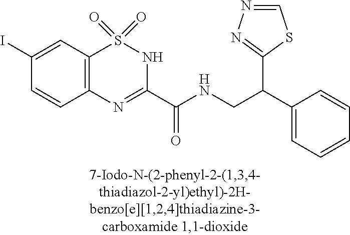 [SMILES: O=C(NCC(c1ccccc1)c1nncs1)C1=Nc2ccc(I)cc2S(=O)(=O)N1] | LCMS-C: Rₜ 2.04 min, m/z 539.8 [M + H]⁺; ¹H NMR (400 MHz, DMSO-d₆) δ 12.7 (br s, 1H), 9.54 (s, 1H), 9.36 (t, J = 5.2 Hz, 1H), 8.07-8.04 (m, 2H), 7.61 (d, J = 8.8 Hz, 1H), 7.39-7.34 (m, 4H), 7.31- 7.29 (m, 1H), 5.09 (t, J = 7.6 Hz, 1H), 4.17-4.10 (m, 1H), 4.04-3.97 (m, 1H). | L | Ester l7 (0.24 mmol), amine l66 (0.24 mmol) EtOH (3 mL) |
| 169 | 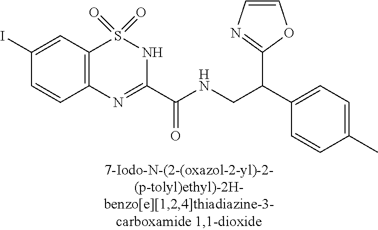 [SMILES: Cc1ccc(C(CNC(=O)C2=Nc3ccc(I)cc3S(=O)(=O)N2)c2ncco2)cc1] | LCMS-C: Rₜ 2.24 min, m/z 536.9 [M + H]⁺; ¹H NMR (400 MHz, DMSO-d₆) δ 12.7 (br s, 1H), 9.27 (t, J = 4.4 Hz, 1H), 8.08-8.02 (m, 3H), 7.59 (d, J = 8.8 Hz, 1H), 7.18-7.13 (m, 5H), 4.63 (t, J = 7.6 Hz, 1H), 4.01-3.93 (m, 1H), 3.88- 3.81 (m, 1H), 2.24 (s, 3H). | L | Ester l7 (0.16 mmol), amine l145 (0.13 mmol) EtOH (3 mL) |
| 170 | 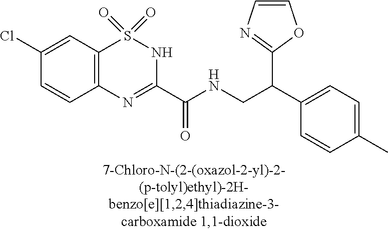 [SMILES: Cc1ccc(C(CNC(=O)C2=Nc3ccc(Cl)cc3S(=O)(=O)N2)c2ncco2)cc1] | LCMS-C: Rₜ 2.19 min, m/z 444.9 [M + H]⁺; ¹H NMR (400 MHz, DMSO-d₆) δ 12.7 (br s, 1H), 9.20 (br s, 1H), 8.03 (s, 1H), 7.86 (s, 1H), 7.77 (s, 2H), 7.18 (s, 1H), 7.16-7.11 (m, 4H), 4.63 (t, J = 7.6 Hz, 1H), 4.03-3.92 (m, 1H), 3.87- 3.80 (m, 1H), 2.24 (s, 3H). | L | Ester l162 (0.34 mmol), amine l145 (0.17 mmol) EtOH (3 mL) |
| 171 | 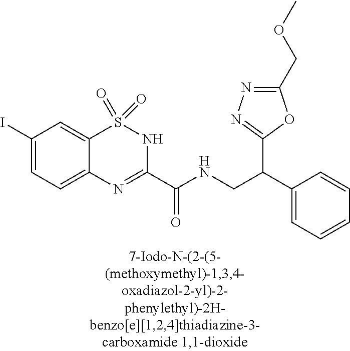 [SMILES: COCc1nnc(C(CNC(=O)C2=Nc3ccc(I)cc3S(=O)(=O)N2)c2ccccc2)o1] | LCMS-C: Rₜ 2.10 min, m/z 567.8 [M + H]⁺; ¹H NMR (400 MHz, DMSO-d₆) δ 12.7 (br s, 1H), 9.40 (t, J = 5.6 Hz, 1H), 8.09-8.04 (m, 2H), 7.60 (d, J = 8.8 Hz, 1H), 7.39-7.29 (m, 5H), 4.80 (t, J = 7.6 Hz, 1H), 4.60 (s, 2H), 4.07-4.00 (m, 1H), 3.91-3.84 (m, 1H), 3.28 (s, 3H). | L | Ester l7 (0.13 mmol), amine l84 (0.13 mmol), Et₃N (1.29 mmol), EtOH (1 mL) Heated at 120° C. overnight |
| 172 | 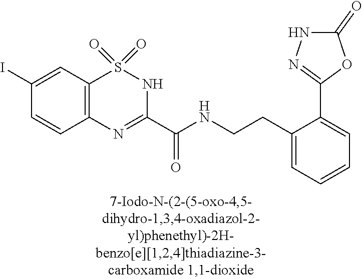 [SMILES: O=C(NCCc1ccccc1-c1n[nH]c(=O)o1)C1=Nc2ccc(I)cc2S(=O)(=O)N1] | LCMS-D: Rₜ 2.00 min, m/z 539.9 [M + H]⁺; ¹H NMR (400 MHz, DMSO-d₆) δ 8.58-8.55 (m, 1H), 7.81 (d, J = 2.0 Hz, 1H), 7.72 (dd, J = 8.8, 2.0 Hz, 1H), 7.62-7.58 (m, 1H), 7.30-7.20 (m, 5H), 3.50-3.44 (m, 2H), 3.28-3.24 (m, 2H). | L | Ester l7 (0.161 mmol), amine l141 (0.146 mmol), MeOH (3 mL) used Diluted reaction mixture with EtOAc and washed with water. Organic layer was dried over Na₂SO₄ and concentrated to give the title compound. |
| 173 | 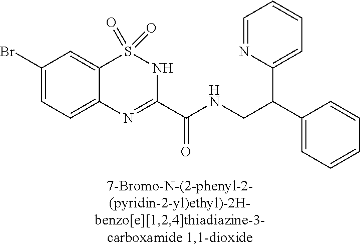 [SMILES: O=C(NCC(c1ccccc1)c1ccccn1)C1=Nc2ccc(Br)cc2S(=O)(=O)N1] | LCMS-C: Rₜ 2.00 min, m/z 485.0 [M + H]⁺; ¹H NMR (400 MHz, DMSO-d₆) δ 12.8 (br s, 1H), 9.25 (t, J = 5.6 Hz, 1H), 8.56 (d, J = 4.0 Hz, 1H), 8.00 (d, J = 2.4 Hz, 1H), 7.93 (dd, J = 8.8, 2.0 Hz, 1H), 7.52-7.69 (m, 2H), 7.36-7.17 (m, 7H), 4.62 (t, J = 7.6 Hz, 1H), 4.04- 4.01 (m, 2H). | L | Ester l5 (0.13 mmol), amine l121 (0.13 mmol) EtOH (2 mL) |
| 174 | 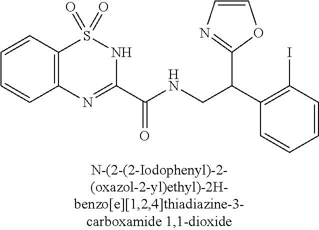 [SMILES: O=C(NCC(c1ncco1)c1ccccc1I)C1=Nc2ccccc2S(=O)(=O)N1] | LCMS-C: Rₜ 2.05 min, m/z 522.9 [M + H]⁺; ¹H NMR (400 MHz, DMSO-d₆) δ 12.7 (br s, 1H), 9.41 (t, J = 5.6 Hz, 1H), 8.05 (s, 1H), 7.90-7.80 (m, 3H), 7.75 (t, J = 8.4 Hz, 1H), 7.52 (t, J = 7.2 Hz, 1H), 7.41 (t, J = 7.2 Hz, 1H), 7.31 (d, J = 7.6 Hz, 1H), 7.20 (s, 1H), 7.06 (t, J = 8.0 Hz, 1H), 5.01 (t, J = 7.6 Hz, 1H), 4.09-4.03 (m, 1H), 3.84- 3.77 (m, 1H). | L | Ester l2 (0.30 mmol), amine l106 (0.30 mmol) EtOH (3 mL) Heated at 120° C. for 3 h Reaction mixture was concentrated, then diluted with EtOAc, washed with water. Organic layer was dried over Na₂SO₄ and |
| | | | | concentrated. |
| | | | | Crude product |
| | | | | was triturated with |
| | | | | hexanes to give |
| | | | | the title |
| | | | | compound. |
| 175 | 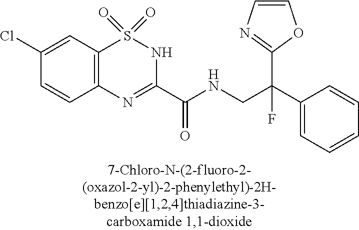 [SMILES: O=C(NCC(F)(c1ccccc1)c1ncco1)C1=Nc2ccc(Cl)cc2S(=O)(=O)N1] | LCMS-C: Rₜ 2.16 min, m/z 448.9 [M + H]⁺; ¹H NMR (400 MHz, DMSO-d₆) δ 12.6 (br s, 1H), 9.06 (t, J = 6.4 Hz, 1H), 8.24 (s, 1H), 7.91 (s, 1H), 7.79 (s, 2H), 7.48- 7.32 (m, 6H), 4.38-4.30 (m, 2H). | L | Ester l162 (0.15 mmol), amine l99 (0.15 mmol) EtOH (2 mL) No Et₃N used Heated at 120° C. overnight A precipitate formed in the reaction. Collected by filtration to give title compound. |
| 176 | 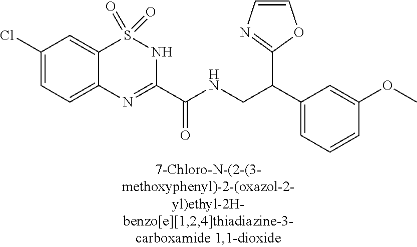 [SMILES: COc1cccc(C(CNC(=O)C2=Nc3ccc(Cl)cc3S(=O)(=O)N2)c2ncco2)c1] | LCMS-C: Rₜ 2.12 min, m/z 461.0 [M + H]⁺; ¹H NMR (400 MHz, DMSO-d₆) δ 12.8 (br s, 1H), 9.30 (t, J = 6.0 Hz, 1H), 8.04 (s, 1H), 7.91 (d, J = 1.6 Hz, 1H), 7.82- 7.81(m, 2H), 7.26-7.20 (m, 2H), 6.85-6.80 (m, 3H), 4.65 (t, J = 7.6 Hz, 1H), 4.03-3.91 (m, 1H), 3.90- 3.85 (m, 1H), 3.71 (s, 3H). | L | Ester l162 (0.50 mmol), amine l96 (0.50 mmol) EtOH (4 mL) No Et₃N used Heated at 120° C. overnight A precipitate formed in the reaction. Collected by filtration to give title compound. |
| 177 | 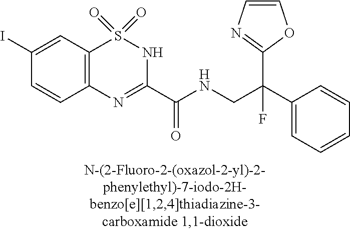 [SMILES: O=C(NCC(F)(c1ccccc1)c1ncco1)C1=Nc2ccc(I)cc2S(=O)(=O)N1] | LCMS-C: Rₜ 2.23 min, m/z 540.9 [M + H]⁺; ¹H NMR (400 MHz, DMSO-d₆) δ 12.7 (br s, 1H), 9.06 (t, J = 5.6 Hz, 1H), 8.24 (s, 1H), 8.10 (s, 1H), 8.07 (d, J = 8.8 Hz, 1H), 7.58 (d, J = 8.4 Hz, 1H), 7.42-7.37 (m, 6H), 4.38-4.32 (m, 2H). | L | Ester l7 (0.20 mmol), amine l99 (0.20 mmol) EtOH (2.5 mL) No Et₃N used Heated at 120° C. overnight A precipitate formed in the reaction. Collected by filtration to give title compound. |
| 178 | 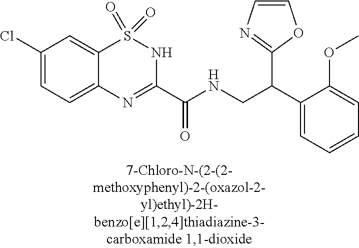 [SMILES: COc1ccccc1C(CNC(=O)C1=Nc2ccc(Cl)cc2S(=O)(=O)N1)c1ncco1] | LCMS-C: Rₜ 2.15 min, m/z 461.0 [M + H]⁺; ¹H NMR (400 MHz, DMSO-d₆) δ 12.8 (br s, 1H), 9.21 (t, J = 6.0 Hz, 1H), 8.00 (s, 1H), 7.90 (d, J = 0.8 Hz, 1H), 7.83-7.77 (m, 2H), 7.28-7.23 (m, 1H), 7.18 (s, 1H), 7.10 (dd, J = 7.6, 1.2 Hz, 1H), 7.01 (d, J = 8.0 Hz, 1H), 6.92 (t, J = 7.6 Hz, 1H), 5.01 (t, J = 7.6 Hz, 1H), 4.03-3.96 (m, 1H), 3.82- 3.75 (m, 1H), 3.32 (s, 3H). | L | Ester l162 (0.37 mmol), amine l76 (0.37 mmol) Et₃N (1.83 mmol) EtOH (2 mL) Heated at 120° C. for 3 h |
| 179 | 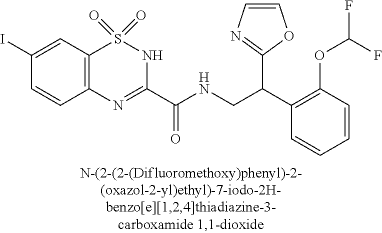 [SMILES: O=C(NCC(c1ncco1)c1ccccc1OC(F)F)C1=Nc2ccc(I)cc2S(=O)(=O)N1] | LCMS-C: Rₜ 2.25 min, m/z 588.9 [M + H]⁺; ¹H NMR (400 MHz, DMSO-d₆) δ 12.7 (br s, 1H), 9.34 (t, J = 6.0 Hz, 1H), 8.11-8.04 (m, 2H), 8.02 (d, J = 0.9 Hz, 1H), 7.60 (d, J = 8.7 Hz, 1H), 7.40-7.34 (m, 2H), 7.27-7.17 (m, 3H), 7.15 (t, J = 73.6 Hz, 1H), 4.99 (t, J = 7.5 Hz, 1H), 4.09-4.02 (m, 1H), 3.85- 3.79 (m, 1H). | L | Ester l7 (0.22 mmol), amine l80 (0.20 mmol) Et₃N (0.98 mmol) EtOH (2 mL) Heated at 120° C. for 3 h |
| 180 | 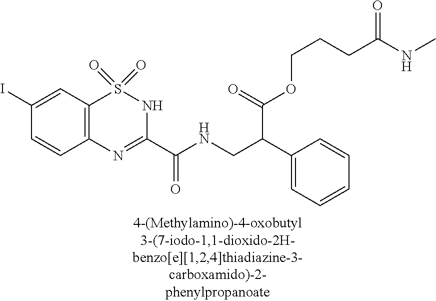 [SMILES: CNC(=O)CCCOC(=O)C(CNC(=O)C1=Nc2ccc(I)cc2S(=O)(=O)N1)c1ccccc1] | LCMS-C: Rₜ 1.98 min, m/z 598.9 [M + H]⁺; ¹H NMR (400 MHz, DMSO-d₆) δ 12.7 (br s, 1H), 9.24 (t, J = 5.6 Hz, 1H), 8.07-8.03 (m, 2H), 7.65 (d, J = 4.0 Hz, 1H), 7.58 (d, J = 8.4 Hz, 1H), 7.36-7.26 (m, 5H), 4.13 (t, J = 7.6 Hz, 1H), 4.04- 3.96 (m, 2H), 3.90-3.83 (m, 1H), 3.66-3.59 (m, 1H), 2.52 (d, J = 4.4 Hz, 3H), 2.05 (t, J = 7.6 Hz, 2H), 1.76-1.68 (m, 2H). | L | Ester l7 (0.65 mmol), amine l72 (0.59 mmol) Et₃N (2.93 mmol) EtOH (2 mL) Heated at 120° C. for 3 h |
| 181 | 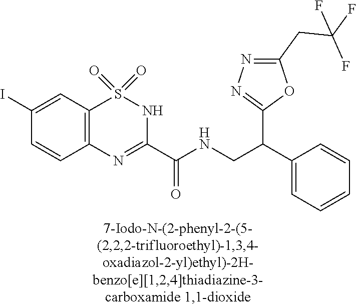 [SMILES: O=C(NCC(c1ccccc1)c1nnc(CC(F)(F)F)o1)C1=Nc2ccc(I)cc2S(=O)(=O)N1] | LCMS-C: Rₜ 2.29 min, m/z 605.9 [M + H]⁺; ¹H NMR (400 MHz, DMSO-d₆) δ 12.7 (br s, 1H), 9.34 (br s, 1H), 8.07-8.03 (m, 2H), 7.57 (d, J = 8.4 Hz, 1H), 7.39-7.31 (m, 5H), 4.81 (t, J = 7.6 Hz, 1H), 4.29 (q, J = 10.8 Hz, 2H), 4.06-3.99 (m, 1H), 3.91-3.86 (m, 1H). | L | Ester l7 (0.10 mmol), amine l102 (0.07 mmol) EtOH (2 mL) No Et₃N used Heated at 120° C. overnight Prep. TLC (DCM/MeOH = 40/1) |
| 182 | 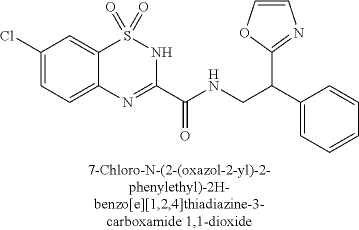 [SMILES: O=C(NCC(c1ccccc1)c1ncco1)C1=Nc2ccc(Cl)cc2S(=O)(=O)N1] | LCMS-C: Rₜ 2.08 min, m/z 431.0 [M + H]⁺; ¹H NMR (400 MHz, DMSO-d₆) δ 12.8 (br s, 1H), 9.32 (t, J = 6.0 Hz, 1H), 8.04 (d, J = 0.8 Hz, 1H), 7.92 (d, J = 1.6 Hz, 1H), 7.84-7.78 (m, 2H), 7.36-7.32 (m, 2H), 7.29-7.25 (m, 3H), 7.20 (d, J = 0.4 Hz, 1H), 4.69 (t, J = 7.6 Hz, 1H), 4.04-3.98 (m, 1H), 3.91-3.85 (m, 1H). | L | Ester l162 (0.35 mmol), amine l27 (0.53 mmol) EtOH (6 mL) Heated at 110° C. for 3 h |
| 183 | 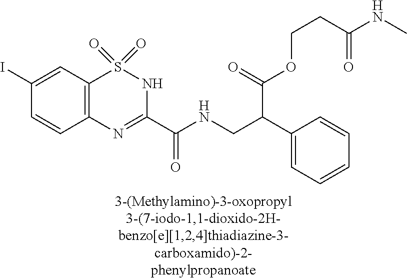 [SMILES: CNC(=O)CCOC(=O)C(CNC(=O)C1=Nc2ccc(I)cc2S(=O)(=O)N1)c1ccccc1] | LCMS-C: Rₜ 1.92 min, m/z 584.9 [M + H]⁺; ¹H NMR (400 MHz, DMSO-d₆) δ 12.7 (br s, 1H), 9.25 (t, J = 5.6 Hz, 1H), 8.09-8.05 (m, 2H), 7.79-7.74 (m, 1H), 7.60 (d, J = 8.8 Hz, 1H), 7.34-7.26 (m, 5H), 4.25 (t, J = 6.0 Hz, 2H), 4.11 (t, J = 7.6 Hz, 1H), 3.87-3.80 (m, 1H), 3.67-3.62 (m, 1H), 2.50 (3H overlap with solvent peak), 2.38 (t, J = 6.0 Hz, 2H). | M | Acid l53 (1.05 mmol), amine l69 (1.05 mmol) HATU (1.74 mmol); DIPEA (5.25 mmol); MeCN (45 mL) |
| 184 | 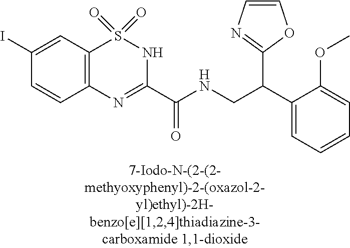 [SMILES: COc1ccccc1C(CNC(=O)C1=Nc2ccc(I)cc2S(=O)(=O)N1)c1ncco1] | LCMS-C: Rₜ 2.22 min, m/z 552.9 [M + H]⁺; ¹H NMR (400 MHz, DMSO-d₆) δ 12.7 (br s, 1H), 9.20 (t, J = 6.0 Hz, 1H), 8.08-8.04 (m, 2H), 7.99 (s, 1H), 7.59 (d, J = 8.8 Hz, 1H), 7.27 (t, J = 8.4 Hz, 1H), 7.18 (s, 1H), 7.10 (dd, 7.6, 1.6 Hz, 1H), 7.01 (d, J = 8.0 Hz, 1H), 6.92 (t, J = 7.6 Hz, 1H), 5.01 (t, J = 7.6 Hz, 1H), 4.02-3.95 (m, 1H), 3.81- 3.76 (m, 1H), 3.73 (s, 3H). | L | Ester l7 (0.25 mmol), amine l76 (0.23 mmol) Et₃N (1.15 mmol) EtOH (2 mL) Heated at 120° C. for 3 h Recrystallised from MeOH |
| 185 | 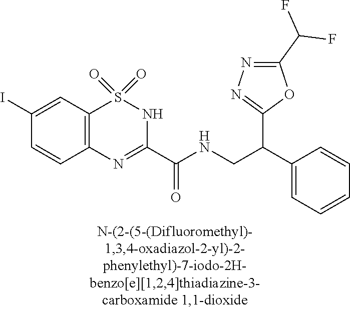 [SMILES: O=C(NCC(c1ccccc1)c1nnc(C(F)F)o1)C1=Nc2ccc(I)cc2S(=O)(=O)N1] | LCMS-D: Rₜ 2.68 min, m/z 573.8 [M + H]⁺; ¹H NMR (400 MHz, DMSO-d₆) δ 12.7 (br s, 1H), 9.39 (t, J = 5.2 Hz, 1H), 8.07 (d, J = 1.9 Hz, 1H), 8.04 (dd, J = 8.7, 2.0 Hz, 1H), 7.56 (d, J = 8.9 Hz, 1H), 7.45-7.32 (m, 6H), 4.87 (t, J = 7.6 Hz, 1H), 4.10-4.03 (m, 1H), 3.92- 3.86 (m, 1H). | M | Acid l53 (0.36 mmol), amine l63 (0.36 mmol) HATU (0.54 mmol) DIPEA (1.44 mmol) MeCN (5 mL) Purified by trituration with MeOH |
| 186 | 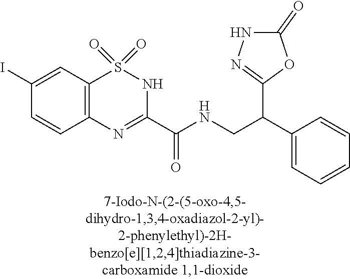 [SMILES: O=C(NCC(c1ccccc1)c1n[nH]c(=O)o1)C1=Nc2ccc(I)cc2S(=O)(=O)N1] | LCMS-D: Rₜ 2.50 min, m/z 539.8 [M + H]⁺; ¹H NMR (400 MHz, DMSO-d₆) δ 12.7 (br s, 1H), 12.3 (s, 1H), 9.40 (t, J = 6.0 Hz, 1H), 8.08-8.04 (m, 2H), 7.60 (d, J = 8.8 Hz, 1H), 7.40-7.30 (m, 5H), 4.36 (t, J = 7.6 Hz, 1H), 3.97-3.90 (m, 1H), 3.75-3.66 (m, 1H). | M | Acid l53 (0.15 mmol), amine l92 (0.20 mmol) HATU (0.23 mmol) DIPEA (0.45 mmol) DMF (5 mL) Prep. TLC (DCM/MeOH = 15/1) |
| 187 | 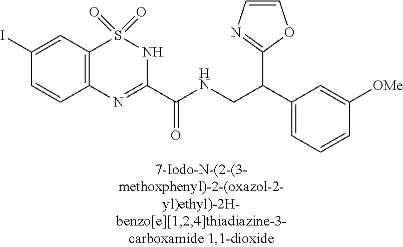 [SMILES: COc1cccc(C(CNC(=O)C2=Nc3ccc(I)cc3S(=O)(=O)N2)c2ncco2)c1] | LCMS-D: Rₜ 2.62 min, m/z 552.9 [M + H]⁺; ¹H NMR (400 MHz, DMSO-d₆) δ 12.7 (br s, 1H), 9.27 (t, J = 6.0 Hz, 1H), 8.08-8.04 (m, 3H), 7.59 (d, J = 8.8 Hz, 1H), 7.26-7.20 (m, 2H), 6.85-6.80 (m, 3H), 4.65 (t, J = 7.6 Hz, 1H), 4.00- 3.94 (m, 1H), 3.91-3.84 (m, 1H), 3.71 (s, 3H). | L | Ester l7 (0.40 mmol), amine l96 (0.48 mmol) EtOH (5 mL) No Et₃N used Heated at 120° C. overnight A precipitate formed in the reaction. Collected by filtration to give title compound. |
| 188 | 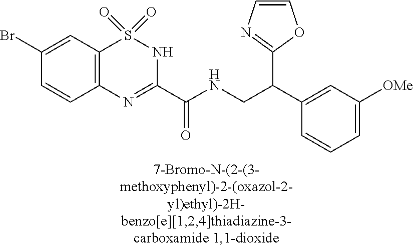 [SMILES: COc1cccc(C(CNC(=O)C2=Nc3ccc(Br)cc3S(=O)(=O)N2)c2ncco2)c1] | LCMS-D: Rₜ 2.61 min, m/z 504.9 [M + H]⁺; ¹H NMR (400 MHz, DMSO-d₆) δ 12.8 (br s, 1H), 9.27 (t, J = 6.0 Hz, 1H), 8.04 (s, 1H), 8.00 (d, J = 2.0 Hz, 1H), 7.93 (dd, J = 8.8, 2.0 Hz, 1H), 7.75 (d, J = 8.8 Hz, 1H), 7.26-7.20 (m, 2H), 6.84-6.80 (m, 3H), 4.65 (t, J = 7.6 Hz, 1H), 4.01-3.94 (m, 1H), 3.91- 3.84 (m, 1H), 3.71 (s, 3H). | L | Ester l5 (0.30 mmol), amine l96 (0.36 mmol) EtOH (4 mL) No Et₃N used A precipitate formed in the reaction. Collected by filtration to give title compound. |
| 189 | 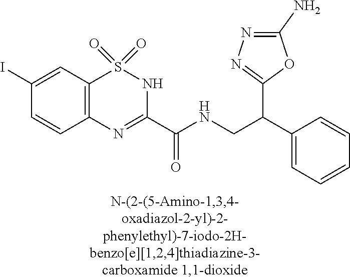 [SMILES: Nc1nnc(C(CNC(=O)C2=Nc3ccc(I)cc3S(=O)(=O)N2)c2ccccc2)o1] | LCMS-D: Rₜ 2.45 min, m/z 538.9 [M + H]⁺; ¹H NMR (400 MHz, DMSO-d₆) δ 12.7 (br s, 1H), 9.30 (t, J = 6.0 Hz, 1H), 8.08-8.04 (m, 2H), 7.60 (d, J = 7.6 Hz, 1H), 7.37-7.27 (m, 5H), 6.94 (s, 2H), 4.53 (t, J = 7.6 Hz, 1H), 3.96-3.90 (m, 1H), 3.84-3.77 (m, 1H). | L | Ester l7 (0.20 mmol), amine l90 (0.24 mmol) EtOH (5 mL) Heated at 120° C. overnight Prep. TLC (DCM/MeOH = 10/1) |
| 190 | 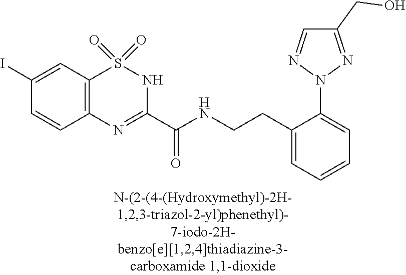 [SMILES: O=C(NCCc1ccccc1-n1ncc(CO)n1)C1=Nc2ccc(I)cc2S(=O)(=O)N1] | LCMS-D: Rₜ 2.57 min, m/z 553.0 [M + H]⁺; ¹H NMR (400 MHz, DMSO-d₆) δ 12.7 (br s, 1H), 9.30 (br s, 1H), 8.10-8.06 (m, 2H), 7.97 (s, 1H), 7.61 (d, J = 8.7 Hz, 1H), 7.57 (s, 4H), 5.37 (br s, 1H), 4.65 (s, 2H), 3.47-3.46 (m, 2H), 2.93 (t, J = 6.4 Hz, 2H). | L | Ester l7 (1.0 mmol), amine l60 (0.92 mmol) EtOH (7 mL) Heated at 120° C. for 4 h |
| 191 | 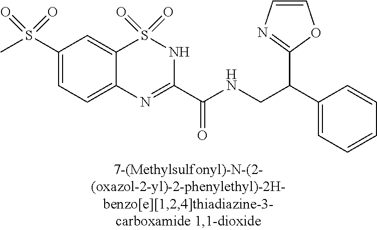 [SMILES: CS(=O)(=O)c1ccc2c(c1)S(=O)(=O)NC(C(=O)NCC(c1ccccc1)c1ncco1)=N2] | LCMS-D: Rₜ 2.29 min, m/z 473.0 [M − H]⁻; ¹H NMR (400 MHz, methanol-d₄) δ 8.42 (d, J = 5.6 Hz, 1H), 8.20 (dd, J = 8.8, 1.6 Hz, 1H), 7.86-7.91 (m, 2H), 7.35-7.25 (m, 5H), 7.17 (s, 1H), 4.65 (t, J = 7.6 Hz, 1H), 4.11-4.06 (m, 1H), 4.01-3.96 (m, 1H), 3.19 (s, 3H). | L | Ester l161 (0.15 mmol), amine l27 (0.30 mmol) EtOH (5 mL) Heated at 110° C. for 2 h |
| 192 | 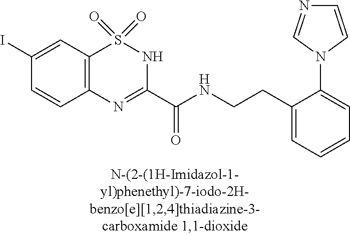 [SMILES: O=C(NCCc1ccccc1-n1ccnc1)C1=Nc2ccc(I)cc2S(=O)(=O)N1] | LCMS-D: Rₜ 2.03 min, m/z 522.1 [M + H]⁺; ¹H NMR (400 MHz, DMSO-d₆) δ 12.5 (br s, 1H), 9.22 (br s, 1H), 8.06-8.02 (m, 2H), 7.83 (s, 1H), 7.57-7.55 (m, 1H), 7.44- 7.39 (m, 4H), 7.30 (br s, 1H), 7.09 (s, 1H), 3.50 (2H obscured by water peak), 2.73 (t, J = 6.8 Hz, 2H). | L | Ester l7 (0.26 mmol), amine l135 (0.214 mmol) EtOH (5 mL) Heated at 130° C. for 3 h |
| 193 | 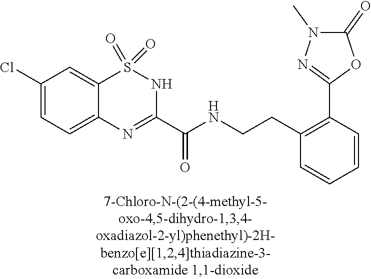 [SMILES: Cn1nc(-c2ccccc2CCNC(=O)C2=Nc3ccc(Cl)cc3S(=O)(=O)N2)oc1=O] | LCMS-C: Rₜ 2.15 min, m/z 461.9 [M + H]⁺; ¹H NMR (400 MHz, DMSO-d₆) δ 12.7 (br s, 1H), 9.29 (t, J = 5.6 Hz, 1H), 7.91 (s, 1H), 7.85-7.79 (m, 2H), 7.71 (d, J = 7.6 Hz, 1H), 7.51 (t, J = 7.2 Hz, 1H), 7.42-7.38 (m, 2H), 3.59-3.50 (m, 2H), 3.42 (s, 3H), 3.24 (t, J = 6.8 Hz, 2H). | M | Acid l163 (0.27 mmol), amine l131 (0.23 mmol) HATU (0.32 mmol) DIPEA (1.15 mmol) DMF (8 mL) |
| 194 | 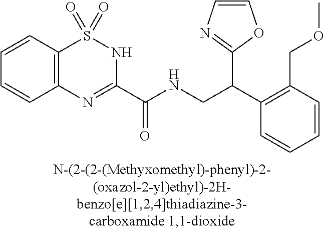 [SMILES: COCc1ccccc1C(CNC(=O)C1=Nc2ccccc2S(=O)(=O)N1)c1ncco1] | LCMS-C: Rₜ 1.85 min, m/z 441.0 [M + H]⁺; ¹H NMR (400 MHz, DMSO-d₆) δ 12.6 (br s, 1H), 9.27 (t, J = 5.6 Hz, 1H), 8.02 (d, J = 0.9 Hz, 1H), 7.84 (dd, J = 8.0, 1.4 Hz, 1H), 7.81 (d, J = 8.3 Hz, 1H), 7.74-7.70 (m, 1H), 7.52 (td, J = 7.7, 1.2 Hz, 1H), 7.37 (dd, J = 7.3, 1.8 Hz, 1H), 7.34-7.23 (m, 3H), 7.20 (d, J = 0.9 Hz, 1H), 4.98 (t, J = 7.6 Hz, 1H), 4.61-4.53 (m, 2H), 4.12-4.01 (m, 1H), 3.83-3.77 (m, 1H), 3.32 (s, 3H). | L | Ester l2 (0.12 mmol), amine l115 (0.10 mmol) EtOH (2 mL) |
| 195 | 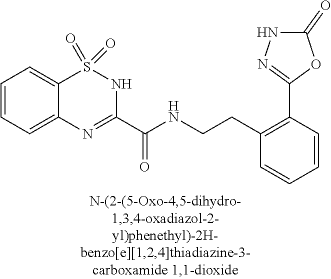 [SMILES: O=C(NCCc1ccccc1-c1n[nH]c(=O)o1)C1=Nc2ccccc2S(=O)(=O)N1] | LCMS-C: Rₜ 1.08 min, m/z 413.9 [M + H]⁺; ¹H NMR (400 MHz, DMSO-d₆) δ 12.6 (br s, 1H), 9.27 (t, J = 6.0 Hz, 1H), 7.86 (dd, J = 8.0, 1.2 Hz, 1H), 7.81 (t, J = 8.0 Hz, 1H), 7.74-7.70 (m, 2H), 7.54- 7.45 (m, 2H), 7.41-7.37 (m, 2H), 3.59-3.54 (m, 2H), 3.25 (t, J = 6.8 Hz, 2H). | M | Acid l29 (0.27 mmol), amine l141 (0.24 mmol) HATU (0.34 mmol) DIPEA (0.82 mmol) DMF (20 mL) Purified by prep. HPLC |
| 196 | 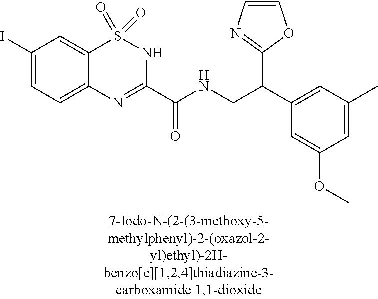 [SMILES: COc1cc(C)cc(C(CNC(=O)C2=Nc3ccc(I)cc3S(=O)(=O)N2)c2ncco2)c1] | LCMS-C: Rₜ 2.27 min, m/z 566.9 [M + H]⁺; ¹H NMR (400 MHz, DMSO-d₆) δ 12.7 (br s, 1H), 9.25- 9.24 (m, 1H), 8.09-8.02 (m, 3H), 7.60-7.57 (m, 1H), 7.20 (d, J = 6.0 Hz, 1H), 6.65-6.62 (m, 3H), 4.63- 4.55 (m, 1H), 4.00-3.92 (m, 1H), 3.88-3.82 (m, 1H), 3.69 (s, 1.5H), 3.68 (s, 1.5H), 2.23 (s, 1.5H), 2.21 (s, 1.5H). | L | Ester l7 (0.52 mmol), amine l150 (0.43 mmol) EtOH (3 mL) Reaction mixture was concentrated, the residue was diluted with water and extracted with EtOAc. Organic extract was washed with brine, dried over Na₂SO₄ and concentrated to give the title compound. |
| 197 | 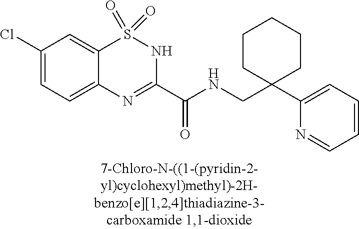 [SMILES: O=C(NCC1(c2ccccn2)CCCCC1)C1=Nc2ccc(Cl)cc2S(=O)(=O)N1] | LCMS-C: Rₜ 1.51 min, m/z 433.0 [M + H]⁺; ¹H NMR (400 MHz, DMSO-d₆) δ 12.7 (br s, 1H), 8.81 (t, J = 6.4 Hz, 1H), 8.58 (dd, J = 4.8, 0.8 Hz, 1H), 7.92 (d, J = 1.6 Hz, 1H), 7.84-7.74 (m, 3H), 7.48 (d, J = 8.0 Hz, 1H), 7.26-7.23 (m, 1H), 3.47 (d, J = 6.4 Hz, 2H), 2.26-2.23 (m, 2H), 1.59-1.54 (m, 4H), 1.42-1.34 (m, 2H), 1.25-1.22 (m, 2H). | L | Ester l162 (0.20 mmol), amine l120 (0.20 mmol) EtOH (3 mL) |
| 198 | 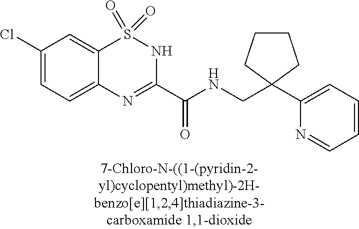 [SMILES: O=C(NCC1(c2ccccn2)CCCC1)C1=Nc2ccc(Cl)cc2S(=O)(=O)N1] | LCMS-C: Rₜ 0.77 min, m/z 418.9 [M + H]⁺; ¹H NMR (400 MHz, DMSO-d₆) δ 12.8 (br s, 1H), 8.91 (t, J = 6.0 Hz, 1H), 8.53 (d, J = 4.0 Hz, 1H), 7.92 (d, J = 2.0 Hz, 1H), 7.84-7.73 (m, 3H), 7.45 (d, J = 8.0 Hz, 1H), 7.26-7.23 (m, 1H), 3.60 (d, J = 6.4 Hz, 2H), 2.02-1.92 (m, 4H), 1.78-1.65 (m, 4H). | L | Ester l162 (0.20 mmol), amine l118 (0.30 mmol) EtOH (2.5 mL) |
| 199 | 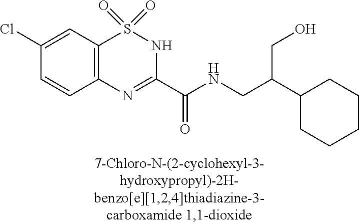 [SMILES: O=C(NCC(CO)C1CCCCC1)C1=Nc2ccc(Cl)cc2S(=O)(=O)N1] | LCMS-C: Rₜ 2.17 min, m/z 400.0 [M + H]⁺; ¹H NMR (400 MHz, DMSO-d₆) δ 12.8 (br s, 1H), 9.12 (t, J = 6.0 Hz, 1H), 7.93 (d, J = 2.4 Hz, 1H), 7.86-7.80 (m, 2H), 4.61 (t, J = 4.8 Hz, 1H), 3.51-3.34 (m, 4H), 3.27-3.20 (m, 1H), 1.69-1.54 (m, 7H), 1.24-1.11 (m, 4H). | L | Ester l162 (1.63 mmol), amine l155 (1.56 mmol) EtOH (20 mL) Heated at 110° C. for 3 h |
| 200 | 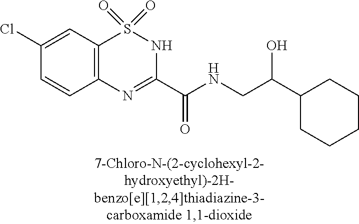 [SMILES: O=C(NCC(O)C1CCCCC1)C1=Nc2ccc(Cl)cc2S(=O)(=O)N1] | LCMS-C: Rₜ 2.06 min, m/z 386.0 [M + H]⁺; ¹H NMR (400 MHz, DMSO-d₆) δ 12.8 (br s, 1H), 8.85 (t, J = 5.6 Hz, 1H), 7.92 (d, J = 2.0 Hz, 1H), 7.86-7.79 (m, 2H), 4.78 (d, J = 5.2 Hz, 1H), 3.46-3.40 (m, 2H), 3.23-3.16 (m, 1H), 1.80-1.58 (m, 5H), 1.17-0.88 (m, 6H). | L | Ester l162 (0.40 mmol), amine l116 (0.40 mmol) EtOH (3 mL) |
| 201 | 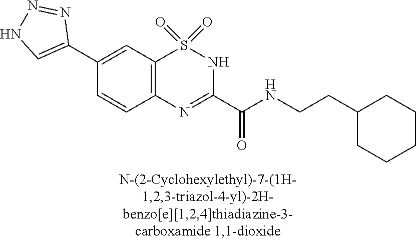 [SMILES: O=C(NCCC1CCCCC1)C1=Nc2ccc(-c3c[nH]nn3)cc2S(=O)(=O)N1] | LCMS-C: Rₜ 2.18 min, m/z 403.0 [M + H]⁺; ¹H NMR (400 MHz, DMSO-d₆) δ 15.3 (br s, 1H), 12.7 (br s, 1H), 9.20 (t, J = 6.0 Hz, 1H), 8.56 (br s, 1H), 8.29 (d, J = 1.2 Hz, 1H), 8.22 (d, J = 8.4 Hz, 1H), 7.80 (d, J = 8.8 Hz, 1H), 3.31-3.26 (m, 2H), 2.06-1.96 (m, 2H), 1.74- 1.58 (m, 5H), 1.46-1.40 (m, 2H), 1.18-1.10 (m, 2H), 0.92-0.84 (m, 2H). | L | Ester l158 (0.25 mmol), amine (0.25 mmol) EtOH (5 mL) Silica gel chromatography (DCM/MeOH = 10/1) |
| 202 | 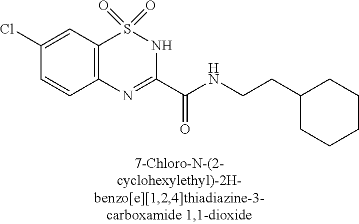 [SMILES: O=C(NCCC1CCCCC1)C1=Nc2ccc(Cl)cc2S(=O)(=O)N1] | LCMS-C: Rₜ 2.40 min, m/z 369.9 [M + H]⁺; ¹H NMR (400 MHz, DMSO-d₆) δ 8.40 (t, J = 6.0 Hz, 1H), 7.55 (d, J = 2.4 Hz, 1H), 7.47 (dd, J = 8.8, 2.4 Hz, 1H), 7.36 (d, J = 8.8 Hz, 1H), 3.22-3.20 (m, 2H), 1.72-1.58 (m, 5H), 1.41-1.36 (m, 2H), 1.23-1.15 (m, 4H), 0.84- 0.83 (m, 2H). | L | Ester l162 (0.35 mmol), amine (0.35 mmol) EtOH (5 mL) |
| 203 | 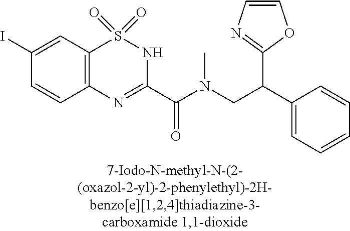 [SMILES: CN(CC(c1ccccc1)c1ncco1)C(=O)C1=Nc2ccc(I)cc2S(=O)(=O)N1] | LCMS-C: Rₜ 2.10 min, m/z 536.9 [M + H]⁺; ¹H NMR (400 MHz, Chloroform-d) δ 10.8 (br s, 0.5H), 10.2 (br s, 0.5H), 8.23 (d, J = 1.8 Hz, 0.5H), 8.21 (d, J = 1.8 Hz, 0.5H), 7.86 (t, J = 1.6 Hz, 0.5H), 7.84 (t, J = 1.7 Hz, 0.5H), 7.66 (s, 0.5H), 7.58 (s, 0.5H), 7.38-7.29 (m, 3.5H), 7.24-7.14 (m, 2H), 7.06-7.04 (m, 1H), 6.92 (d, J = 8.6 Hz, 0.5H), 4.75-4.51 (m, 2H), 4.24-4.19 (m, 0.5H), 4.02-3.96 (m, 0.5H), 3.23 (s, 1.5H), 3.01 (s, 1.5H). | M | Acid l53 (0.54 mmol), amine l133 (0.49 mmol) HATU (0.74 mmol) DIPEA (1.48 mmol) DMF (7 mL) |
| 204 | 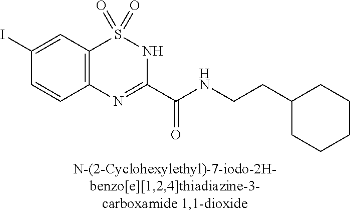 [SMILES: O=C(NCCC1CCCCC1)C1=Nc2ccc(I)cc2S(=O)(=O)N1] | LCMS-C: Rₜ 3.12 min, m/z 461.9 [M + H]⁺; ¹H NMR (400 MHz, DMSO-d₆) δ 12.7 (br s, 1H), 9.20 (br s, 1H), 8.24-8.01 (m, 2H), 7.61 (s, 1H), 3.30-3.20 (m, 2H), 1.88- 0.78 (m, 13H). | M | Ester l7 (0.39 mmol), amine (0.43 mmol) MeOH (10 mL) used Heated at 120° C. overnight Most of the solvent was removed and residue adjusted to pH 5 with 1 M aqueous HCl. |
| | | | | Resulting |
| | | | | precipitate was |
| | | | | collected to give |
| | | | | the title |
| | | | | compound. |
| 205 | 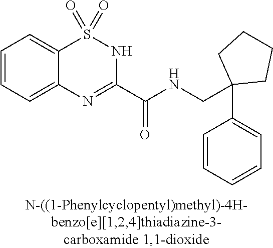 [SMILES: O=C(NCC1(c2ccccc2)CCCC1)C1=Nc2ccccc2S(=O)(=O)N1] | LCMS-B: rt 3.772 min, m/z 384.2 [M + H]⁺ | N | Ester l2 (0.197 mmol), amine (0.197 mmol) Reaction was cooled and the solvent removed in vacuo to give the title compound. |
| 206 | 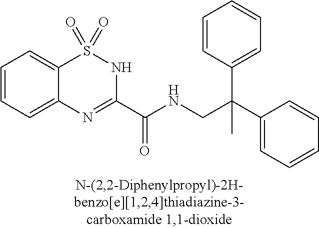 [SMILES: CC(CNC(=O)C1=Nc2ccccc2S(=O)(=O)N1)(c1ccccc1)c1ccccc1] | LCMS-B: rt 3.386 min, m/z 420.2 [M + H]⁺ | N | Ester l2 (0.197 mmol), amine (0.236 mmol) Reaction was cooled and the resulting precipitate was collected and washed with a portion of EtOH (2 mL) and dried under vacuum to give the title |
| | | | | compound. |
| 207 | 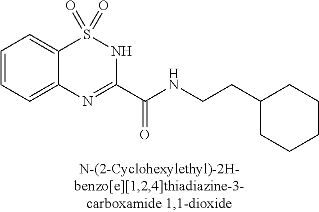 [SMILES: O=C(NCCC1CCCCC1)C1=Nc2ccccc2S(=O)(=O)N1] | LCMS-B: rt 3.717 min, m/z 336.2 [M + H]⁺; ¹H NMR (400 MHz, DMSO-d₆): δ 12.61 (s, 1H), 9.20 (t, J = 5.9 Hz, 1H), 7.84 (ddd, J = 14.3, 8.2, 1.3 Hz, 2H), 7.73 (ddd, J = 8.5, 7.3, 1.5 Hz, 1H), 7.52 (ddd, J = 8.2, 7.3, 1.2 Hz, 1H), 3.32-3.25 (m, 2H), 1.73 (d, J = 12.5 Hz, 2H), 1.69-1.56 (m, 3H), 1.43 (q, J = 7.0, 7.0 Hz, 2H), 1.33-1.09 (m, 4H), 0.95-0.81 (m, 2H). | N | Ester l2 (0.098 mmol), amine (0.098 mmol), Et₃N (0.098 mmol) Reaction was cooled, precipitate was collected by filtration, washed with EtOH (2 mL) and then dried under vacuum to give the title |
| | | | | compound. |
| 133 | 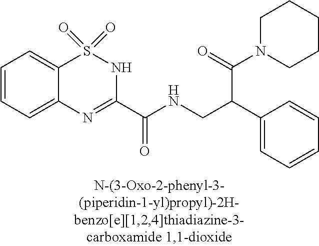 [SMILES: O=C(NCC(C(=O)N1CCCCC1)c1ccccc1)C1=Nc2ccccc2S(=O)(=O)N1] | LCMS (ES-API): Rₜ 2.80 min, m/z 441.1 [M + H]⁺; ¹H NMR (400 MHz, d₆-DMSO) δ 12.6 (s, 1H), 8.24 (t, J = 6.4 Hz, 1H), 7.97 (dd, J = 8.0. 1.2 Hz, 1H), 7.60 (m, 1H), 7.48 (m, 1H), 7.37-7.33 (m, 2H), 7.30-7.25 (m, 4H), 4.02-3.83 (m, 4H), 3.30-3.24 (m, 2H), 3.18-3.12 (m, 1H), 1.50- 1.22 (m, 6H). | O | |
| 134 | 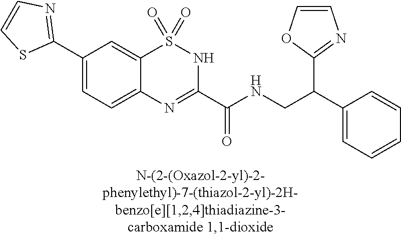 [SMILES: O=C(NCC(c1ccccc1)c1ncco1)C1=Nc2ccc(-c3nccs3)cc2S(=O)(=O)N1] | LCMS (ES-API): R, 2.58 min, m/z 480.1 [M + H]⁺; ¹H NMR (400 MHz, d₆-DMSO) δ 12.9 (s, 1H), 9.18 (s, 1H), 8.26- 8.21 (m, 2H), 8.05 (s, 1H), 7.98 (s, 1H), 7.87-7.81 (m, 2H), 7.35- 7.21 (m, 6H), 4.68 (t, J = 6.8 Hz, 1H), 4.04-3.87 (m, 2H). | P | |
| 135 | 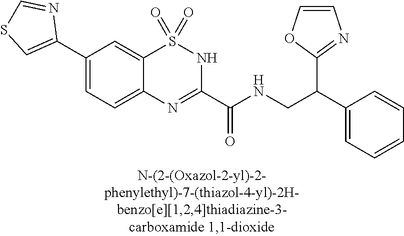 [SMILES: O=C(NCC(c1ccccc1)c1ncco1)C1=Nc2ccc(-c3cscn3)cc2S(=O)(=O)N1] | LCMS (ES-API): Rt 2.49 min, m/z 480.1 [M + H]⁺; ¹H NMR (400 MHz, d₆-DMSO) δ 12.8 (s, 1H), 9.25 (s, 2H), 8.41 (s, 2H), 8.33 (d, J = 5.6 Hz, 1H), 8.05 (s, 1H), 7.85 (d, J = 6.8 Hz, 1H), 7.35-7.28 (m, 5H), 7.21 (s, 1H), 4.68 (t, J = 6.8 Hz, 1H), 4.04-3.88 (m, 2H). | P | |

### Example 208: 7-(1-Aminoethyl)-N-(2-(oxazol-2-yl)-2-phenylethyl)-2H-benzo[e][1,2,4]thiadiazine-3-carboxamide 1,1-dioxide 208

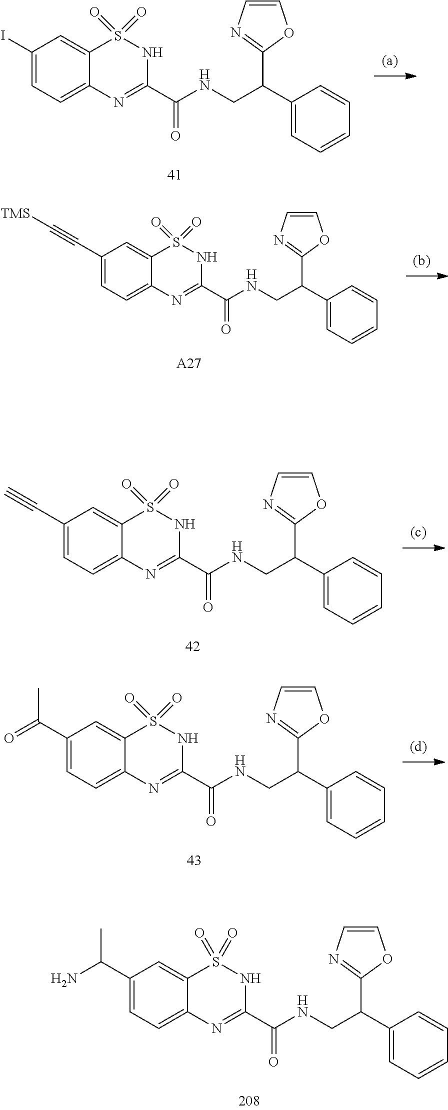

### a) N-(2-(Oxazol-2-yl)-2-phenylethyl)-7-((trimethylsilyl)ethynyl)-2H-benzo[e][1,2,4]thiadiazine-3-carboxamide 1,1-dioxide A27

To a mixture of 7-iodo-N-(2-(oxazol-2-yl)-2-phenylethyl)-2H-benzo[e][1,2,4]thiadiazine-3-carboxamide 1,1-dioxide 41 (880 mg, 1.7 mmol), Pd(PPh₃)₂Cl₂(120 mg, 0.17 mmol) and CuI (32 mg, 0.17 mmol) in Et₃N (10 mL) and DMF (10 mL) under N₂ was added ethynyltrimethylsilane (700 mg, 6.8 mmol) and the mixture was stirred at RT under N₂ overnight. The mixture was concentrated under reduced pressure and the residue was dissolved in EtOAc (200 mL), washed with water (100 mL×3), dried over Na₂SO₄, filtered and concentrated under reduced pressure to give the title compound (1.3 g, >100%) as a brown solid, which was used directly in the next step. LCMS-D: Rₜ 3.19 min, m/z 493.1 [M+H]⁺.

### b) 7-Ethynyl-N-(2-(oxazol-2-yl)-2-phenylethyl)-2H-benzo[e][1,2,4]thiadiazine-3-carboxamide 1,1-dioxide 42

To a solution of N-(2-(oxazol-2-yl)-2-phenylethyl)-7-((trimethylsilyl)ethynyl)-2H-benzo[e][1,2,4]thiadiazine-3-carboxamide 1,1-dioxide A27 (1.2 g, 2.4 mmol) in THF (20 mL) and MeOH (20 mL) was added a 1 M aqueous KOH solution (12.0 mL, 12.0 mmol) and the mixture was stirred at RT for 45 min. Dowex 50WX8 H⁺ form (50 g) was added and stirring was continued for 30 min. The mixture was filtered and the filtrate was concentrated under reduced pressure. The residue was diluted with EtOAc (100 mL) and concentrated under reduced pressure to give the title compound (800 mg, 80%) as a brown solid. LCMS-D: Rₜ 2.64 min, m/z 421.1 [M+H]⁺.

### c) 7-Acetyl-N-(2-(oxazol-2-yl)-2-phenylethyl)-2H-benzo[e][1,2,4]thiadiazine-3-carboxamide 1,1-dioxide 43

A suspension of AgSbF₆ (69 mg, 0.2 mmol) and chloro[1,3-bis(2,6-diisopropylphenyl)imidazol-2-ylidene]gold(I) (124 mg, 0.2 mmol) in MeOH (12 mL) was stirred at RT for 2 min. 7-Ethynyl-N-(2-(oxazol-2-yl)-2-phenylethyl)-2H-benzo[e][1,2,4]thiadiazine-3-carboximide 1,1-dioxide 42 (420 mg, 1.0 mmol) and water (6 mL) were then added and the mixture was heated at 65° C. overnight. The resulting precipitate was collected by filtration and dried to give the title compound (400 mg, 90%) as a brown solid, which was used in the next step without further purification. LCMS-D: Rₜ 1.77 min, m/z 439.1 [M+H]⁺.

### d) 7-(1-Aminoethyl)-N-(2-(oxazol-2-yl)-2-phenylethyl)-2H-benzo[e][1,2,4]thiadiazine-3-carboxamide 1,1-dioxide 208

To a solution of 7-acetyl-N-(2-(oxazol-2-yl)-2-phenylethyl)-2H-benzo[e][1,2,4]thiadiazine-3-carboxamide 43 (219 mg, 0.5 mmol) in MeOH (5 mL) was added NH₄OAc (385 mg, 5 mmol) and NaCNBH₃ (32 mg, 0.5 mmol) and the mixture was heated at reflux for 18 h. The mixture was diluted with water, extracted with EtOAc (100 mL) and the organic layer was concentrated under reduced pressure. The residue was purified by prep. TLC (DCM/MeOH=10/1) to give the title compound (50 mg, 25%) as a yellow solid. LCMS-D: Rₜ 1.89 min, m/z 440.1 [M+H]⁺. ¹H NMR (400 MHz, Methanol-d₄) δ 7.93 (s, 1H), 7.85 (s, 1H), 7.72-7.65 (m, 1H), 7.57 (d, J=8.9 Hz, 1H), 7.36-7.24 (m, 5H), 7.17 (s, 1H), 4.65-4.61 (m, 1H), 4.54 (q, J=6.7 Hz, 1H), 4.11-4.03 (m, 1H), 4.01-3.92 (m, 1H), 1.63 (d, J=6.9 Hz, 3H).

### Example 209: 7-(1-(Methylamino)ethyl)-N-(2-(oxazol-2-yl)-2-phenylethyl)-2H-benzo[e][1,2,4]thiadiazine-3-carboxamide 1,1-dioxide 209

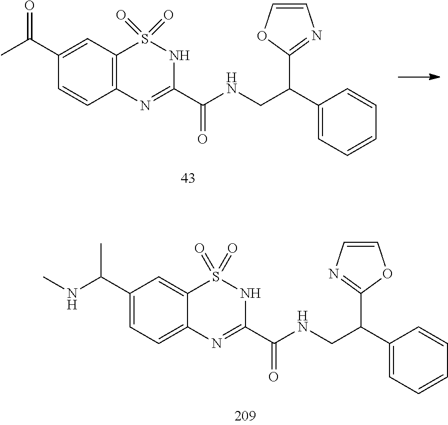 [SMILES: CC(=O)c1ccc2c(c1)S(=O)(=O)NC(C(=O)NCC(c1ccccc1)c1ncco1)=N2 ; CNC(C)c1ccc2c(c1)S(=O)(=O)NC(C(=O)NCC(c1ccccc1)c1ncco1)=N2]

To a solution of 7-acetyl-N-(2-(oxazol-2-yl)-2-phenylethyl)-2H-benzo[e][1,2,4]thiadiazine-3-carboxamide 1,1-dioxide 43 (44 mg, 0.1 mmol) in MeOH (5 mL) was added CH₃NH₂(2 M solution in THF, 0.5 mL, 1.0 mmol) and NaBH₃CN (6.3 mg, 0.1 mmol). The flask was sealed and the mixture was heated at 66° C. overnight. The mixture was diluted with water, extracted with EtOAc and the organic extract was concentrated under reduced pressure. The residue was purified by prep. TLC (DCM/MeOH=10/1) to give the title compound (10 mg, 20%) as a yellow solid. LCMS-D: Rₜ 2.09 min, m/z 454.2 [M+H]⁺. ¹H NMR (400 MHz, Methanol-d₄) δ 8.02 (s, 1H), 7.86 (s, 1H), 7.85-7.80 (m, 1H), 7.73-7.67 (m, 1H), 7.37-7.25 (m, 5H), 7.17 (s, 1H), 4.66-4.58 (m, 1H), 4.46 (q, J=6.8 Hz, 1H), 4.12-4.04 (m, 1H), 4.02-3.93 (m, 1H), 2.60 (s, 3H), 1.69 (d, J=6.9 Hz, 3H).

### Example 210: 7-(1-(Methylsulfonamido)ethyl)-N-(2-(oxazol-2-yl)-2-phenylethyl)-2H-benzo[e][1,2,4]thiadiazine-3-carboxamide 1,1-dioxide 210

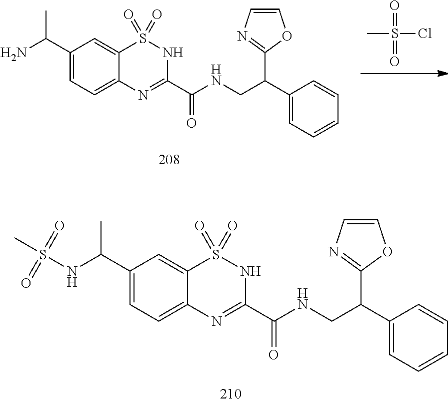

To a solution of 7-(1-aminoethyl)-N-(2-(oxazol-2-yl)-2-phenylethyl)-2H-benzo[e][1,2,4]thiadiazine-3-carboxamide 1,1-dioxide 208 (44 mg, 0.1 mmol) in pyridine (5 mL) at 0° C. was added MsCl (51 mg, 0.5 mmol) and the mixture was stirred at RT overnight. The mixture was diluted with 1 M aqueous HCl (20 mL), extracted with EtOAc (100 mL) and the organic extract was washed with water (50 mL×3) and concentrated under reduced pressure. The residue was purified by prep. TLC (DCM/MeOH=10/1) to give the title compound (20 mg, 40%) as a yellow solid. LCMS-D: Rₜ 2.18 min, m/z 518.1 [M+H]⁺. ¹H NMR (400 MHz, Methanol-d₄) δ 7.92 (d, J=2.0 Hz, 1H), 7.85 (d, J=0.9 Hz, 1H), 7.73 (dd, J=8.7, 2.0 Hz, 1H), 7.59 (d, J=8.7 Hz, 1H), 7.36-7.24 (m, 5H), 7.17 (d, J=0.9 Hz, 1H), 4.70 (q, J=7.0 Hz, 1H), 4.62 (t, 7.6 Hz, 1H), 4.12-4.03 (m, 1H), 4.01-3.94 (m, 1H), 2.78 (s, 3H), 1.52 (d, J=7.0 Hz, 3H).

### Example 211: 7-(1-Acetamidoethyl)-N-(2-(oxazol-2-yl)-2-phenylethyl)-2H-benzo[e][1,2,4]thiadiazine-3-carboxamide 1,1-dioxide 211

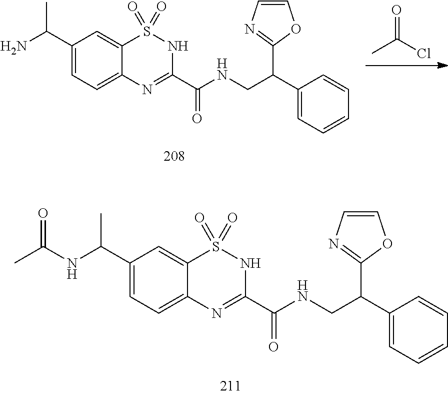

To a solution of 7-(1-aminoethyl)-N-(2-(oxazol-2-yl)-2-phenylethyl)-2H-benzo[e][1,2,4]thiadiazine-3-carboxamide 1,1-dioxide 208 (44 mg, 0.1 mmol) in pyridine (5 mL) at 0° C. was added acetyl chloride (78 mg, 1.0 mmol) and the mixture was stirred at RT overnight. The mixture was diluted with 1 M aqueous HCl (20 mL), extracted with EtOAc (100 mL) and the organic extract was washed with water (50 mL×3) and concentrated under reduced pressure. The residue was purified by prep. TLC (DCM/MeOH=10/1) to give the title compound (10 mg, 20%) as a white solid. LCMS-D: Rₜ 2.29 min, m/z 482.0 [M+H]⁺. ¹H NMR (400 MHz, Methanol-d₄) δ 7.86 (s, 1H), 7.81 (d, J=2.0 Hz, 1H), 7.66 (dd, J=8.6, 2.0 Hz, 1H), 7.56 (d, J=8.6 Hz, 1H), 7.31 (s, 5H), 7.17 (d, J=0.8 Hz, 1H), 5.05 (q, J=7.0 Hz, 1H), 4.62 (t, J=7.6 Hz, 1H), 4.11-4.03 (m, 1H), 4.01-3.93 (m, 1H), 1.98 (s, 3H), 1.46 (d, J=7.0 Hz, 3H).

### Example 212: 7-(1-Hydroxyethyl)-N-(2-(oxazol-2-yl)-2-phenylethyl)-2H-benzo[e][1,2,4]thiadiazine-3-carboxamide 1,1-dioxide 212

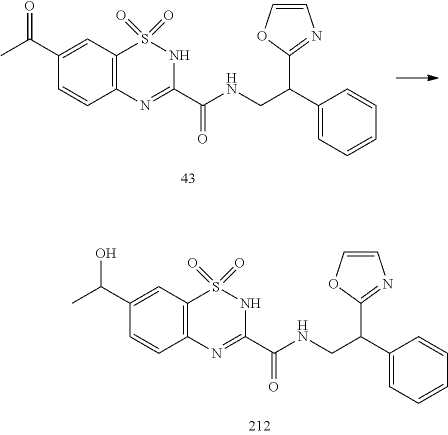 [SMILES: CC(=O)c1ccc2c(c1)S(=O)(=O)NC(C(=O)NCC(c1ccccc1)c1ncco1)=N2 ; CC(O)c1ccc2c(c1)S(=O)(=O)NC(C(=O)NCC(c1ccccc1)c1ncco1)=N2]

To a solution of 7-acetyl-N-(2-(oxazol-2-yl)-2-phenylethyl)-2H-benzo[e][1,2,4]thiadiazine-3-carboxamide 1,1-dioxide 43 (44 mg, 0.1 mmol) in MeOH (5 mL) was added NaBH₄ (4.5 mg, 0.12 mmol) and the mixture was stirred at RT under N₂ for 1 h. The mixture was diluted with water, extracted with EtOAc and the organic extract was concentrated under reduced pressure. The residue was purified by prep. TLC (DCM/MeOH=10/1) to give the title compound (10 mg, 20%) as a yellow solid. LCMS-D: Rₜ 2.4 min, m/z 441.1 [M+H]⁺. ¹H NMR (400 MHz, DMSO-d₆) δ 12.6 (s, 1H), 9.26 (t, J=6.0 Hz, 1H), 8.04 (d, J=0.9 Hz, 1H), 7.78 (d, J=1.8 Hz, 1H), 7.76-7.72 (m, 1H), 7.69-7.64 (m, 1H), 7.37-7.31 (m, 2H), 7.30-7.24 (m, 3H), 7.20 (d, J=0.9 Hz, 1H), 5.44 (d, J=4.4 Hz, 1H), 4.84-4.77 (m, 1H), 4.68 (t, J=7.5 Hz, 1H), 4.05-3.96 (m, 1H), 3.92-3.84 (m, 1H), 1.33 (d, J=6.4 Hz, 3H).

### Example 213: N-(2-(Oxazol-2-yl)-2-phenylethyl)-7-(1H-1,2,3-triazol-4-yl)-2H-benzo[e][1,2,4]thiadiazine-3-carboxamide 1,1-dioxide 213

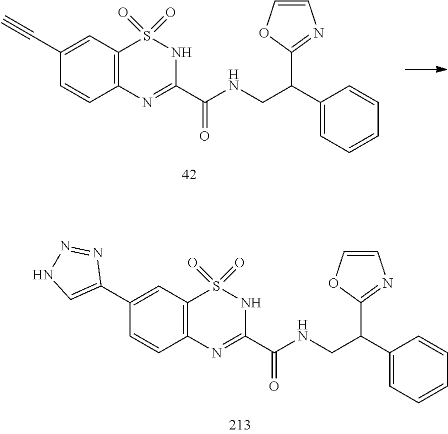 [SMILES: C#Cc1ccc2c(c1)S(=O)(=O)NC(C(=O)NCC(c1ccccc1)c1ncco1)=N2 ; O=C(NCC(c1ccccc1)c1ncco1)C1=Nc2ccc(-c3c[nH]nn3)cc2S(=O)(=O)N1]

To a solution of 7-ethynyl-N-(2-(oxazol-2-yl)-2-phenylethyl)-2H-benzo[e][1,2,4]thiadiazine-3-carboxamide 1,1-dioxide 42 (52 mg, 0.12 mmol) in DMF (1 mL) and EtOH (0.25 mL) was added CuI (5 mg, 24 μmol) and azidotrimethylsilane (18 mg, 0.15 mmol) and the mixture was stirred at 120° C. for 18 h in a sealed tube. The mixture was treated with 1 M aqueous HCl (1 mL), diluted with EtOAc (100 mL) and washed with water (50 mL×3). The organic layer was concentrated under reduced pressure and the residue was purified by prep. TLC (DCM/MeOH=20/1) to give the title compound (20 mg, 40%) as a yellow solid. LCMS-D: Rₜ 2.4 min, m/z 464.1 [M+H]⁺. ¹H NMR (400 MHz, DMSO-d₆) δ 15.5-15.2 (m, 1H), 12.7 (s, 1H), 9.28 (t, J=6.0 Hz, 1H), 8.81-8.38 (m, 1H), 8.29 (s, 1H), 8.22 (d, J=8.4 Hz, 1H), 8.05 (s, 1H), 7.88 (d, J=8.7 Hz, 1H), 7.39-7.31 (m, 2H), 7.31-7.24 (m, 3H), 7.21 (s, 1H), 4.68 (t, J=7.5 Hz, 1H), 4.07-3.97 (m, 1H), 3.95-3.84 (m, 1H).

### Example 214: 7-Bromo-N-(2-(3-hydroxyphenyl)-2-(oxazol-2-yl)ethyl)-2H-benzo[e][1,2,4]thiadiazine-3-carboxamide 1,1-dioxide 214

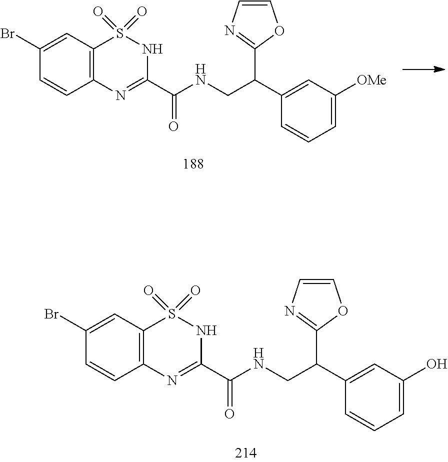 [SMILES: COc1cccc(C(CNC(=O)C2=Nc3ccc(Br)cc3S(=O)(=O)N2)c2ncco2)c1 ; O=C(NCC(c1cccc(O)c1)c1ncco1)C1=Nc2ccc(Br)cc2S(=O)(=O)N1]

To a solution of 7-bromo-N-(2-(3-methoxyphenyl)-2-(oxazol-2-yl)ethyl)-2H-benzo[e][1,2,4]thiadiazine-3-carboxamide 1,1-dioxide 188 (101 mg, 0.2 mmol) in DCM (4 mL) at 0° C. was added BBr₃ (1 M solution in DCM, 0.4 mL, 0.4 mmol) and the mixture was stirred overnight. The mixture was diluted with DCM (50 mL), washed with a saturated aqueous NaHCO₃ solution and concentrated under reduced pressure. The residue was purified by prep. TLC (DCM/MeOH=20/1) to give the title compound (10 mg, 10%) as a yellow solid. LCMS-D: Rₜ 2.41 min, m/z 490.8 [M+H]⁺. ¹H NMR (400 MHz, DMSO-d₆) δ 12.8 (s, 1H), 9.44 (s, 1H), 9.27 (t, J=5.9 Hz, 1H), 8.04 (d, J=0.9 Hz, 1H), 8.00 (d, J=2.2 Hz, 1H), 7.93 (dd, J=8.9, 2.2 Hz, 1H), 7.75 (d, J=8.9 Hz, 1H), 7.19 (d, J=0.9 Hz, 1H), 7.15-7.08 (m, 1H), 6.71-6.62 (m, 3H), 4.57 (t, J=7.5 Hz, 1H), 4.02-3.93 (m, 1H), 3.86-3.77 (m, 1H).

### Example 215: N-(2-(3-Hydroxyphenyl)-2-(oxazol-2-yl)ethyl)-7-iodo-2H-benzo[e][1,2,4]thiadiazine-3-carboxamide 1,1-dioxide 215

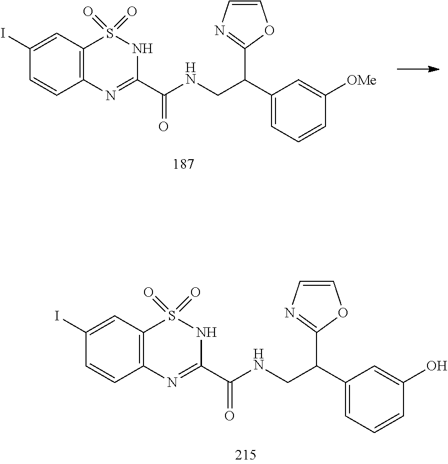 [SMILES: COc1cccc(C(CNC(=O)C2=Nc3ccc(I)cc3S(=O)(=O)N2)c2ncco2)c1 ; O=C(NCC(c1cccc(O)c1)c1ncco1)C1=Nc2ccc(I)cc2S(=O)(=O)N1]

To a solution of 7-iodo-N-(2-(3-methoxyphenyl)-2-(oxazol-2-yl)ethyl)-2H-benzo[e][1,2,4]thiadiazine-3-carboxamide 1,1-dioxide 187 (110 mg, 0.2 mmol) in DCM (10 mL) at 0° C. was added BBr₃ (1 M solution in DCM, 0.4 mL, 0.4 mmol) and the mixture was stirred overnight. The mixture was diluted with DCM (100 mL), washed with a saturated aqueous NaHCO₃ solution (50 mL), dried over Na₂SO₄, filtered and concentrated under reduced pressure. The residue was purified by prep. TLC (DCM/MeOH=20/1) to give the title compound (20 mg, 20%) as a white solid. LCMS-D: Rₜ 2.44 min, m/z 538.9 [M+H]⁺. ¹H NMR (400 MHz, DMSO-d₆) δ 12.7 (s, 1H), 9.45 (s, 1H), 9.21 (s, 1H), 8.09-8.01 (m, 3H), 7.55 (d, J=8.8 Hz, 1H), 7.19 (s, 1H), 7.15-7.07 (m, 1H), 6.72-6.61 (m, 3H), 4.56 (t, J=7.5 Hz, 1H), 4.01-3.92 (m, 1H), 3.85-3.76 (m, 1H).

### Example 216: 7-Chloro-N-(2-(3-hydroxyphenyl)-2-(oxazol-2-yl)ethyl)-2H-benzo[e][1,2,4]thiadiazine-3-carboxamide 1,1-dioxide 216

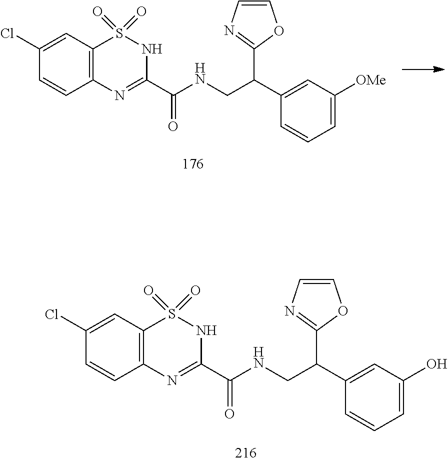 [SMILES: COc1cccc(C(CNC(=O)C2=Nc3ccc(Cl)cc3S(=O)(=O)N2)c2ncco2)c1 ; O=C(NCC(c1cccc(O)c1)c1ncco1)C1=Nc2ccc(Cl)cc2S(=O)(=O)N1]

To a solution of 7-chloro-N-(2-(3-methoxyphenyl)-2-(oxazol-2-yl)ethyl)-2H-benzo[e][1,2,4]thiadiazine-3-carboxamide 1,1-dioxide 176 (60 mg, 0.13 mmol) in DCM (10 mL) at 0° C. was added BBr₃ (1 M solution in DCM, 0.4 mL, 0.4 mmol) and the reaction was stirred overnight. The mixture was diluted with DCM (100 mL), washed with water (×3), dried over Na₂SO₄, filtered and concentrated under reduced pressure. The residue was rinsed with MeOH (2 mL) and dried to give the title compound (25 mg, 40%) as a grey solid. LCMS-C: Rₜ 2.30 min, m/z 446.9 [M+H]⁺. ¹H NMR (400 MHz, DMSO-d₆) δ 12.8 (s, 1H), 9.45 (s, 1H), 9.29 (t, J=5.9 Hz, 1H), 8.04 (s, 1H), 7.92 (s, 1H), 7.87-7.78 (m, 2H), 7.20 (s, 1H), 7.11 (t, J=7.7 Hz, 1H), 6.72-6.62 (m, 3H), 4.57 (t, J=7.5 Hz, 1H), 4.03-3.92 (m, 1H), 3.88-3.75 (m, 1H).

### Example 217: N-(2-(3-(Cyclopropylmethoxy)phenyl)-2-(oxazol-2-yl)ethyl)-7-iodo-2H-benzo[e][1,2,4]thiadiazine-3-carboxamide 1,1-dioxide 217

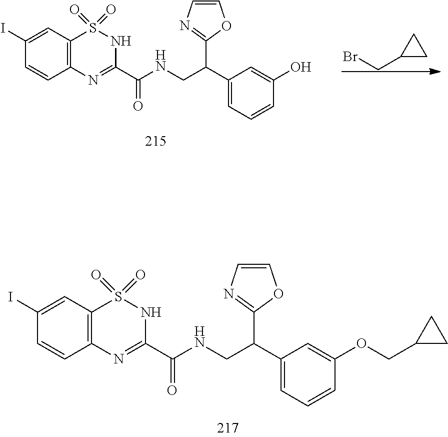 [SMILES: O=C(NCC(c1cccc(O)c1)c1ncco1)C1=Nc2ccc(I)cc2S(=O)(=O)N1 ; BrCC1CC1 ; O=C(NCC(c1cccc(OCC2CC2)c1)c1ncco1)C1=Nc2ccc(I)cc2S(=O)(=O)N1]

To a solution of N-(2-(3-hydroxyphenyl)-2-(oxazol-2-yl)ethyl)-7-iodo-2H-benzo[e][1,2,4]thiadiazine-3-carboxamide 1,1-dioxide 215 (160 mg, 0.3 mmol) in CH₃CN (15 mL) was added Ag₂O (348 mg, 1.5 mmol) and (bromomethyl)cyclopropane (400 mg, 3.0 mmol) and the mixture was stirred at RT under N₂ overnight. The mixture was diluted with DCM (100 mL), washed with water, dried over Na₂SO₄, filtered and concentrated under reduced pressure. The residue was purified by prep. TLC (DCM/MeOH=20/1) to give the title compound (20 mg, 10%) as a yellow solid. LCMS-D: Rₜ 2.39 min, m/z 592.9 [M+H]⁺. ¹H NMR (400 MHz, DMSO-d₆) δ 12.7 (s, 1H), 9.26 (t, J=6.0 Hz, 1H), 8.11-8.01 (m, 3H), 7.58 (d, J=8.7 Hz, 1H), 7.25-7.18 (m, 2H), 6.83-6.76 (m, 3H), 4.61 (t, J=7.5 Hz, 1H), 4.00-3.92 (m, 1H), 3.91-3.82 (m, 1H), 3.80-3.69 (m, 2H), 0.86-0.80 (m, 1H), 0.54-0.48 (m, 2H), 0.29-0.23 (m, 2H).

### Example 218: N-(2-(2-Cyanophenyl)-2-(oxazol-2-yl)ethyl)-2H-benzo[e][1,2,4]thiadiazine-3-carboxamide 1,1-dioxide 218

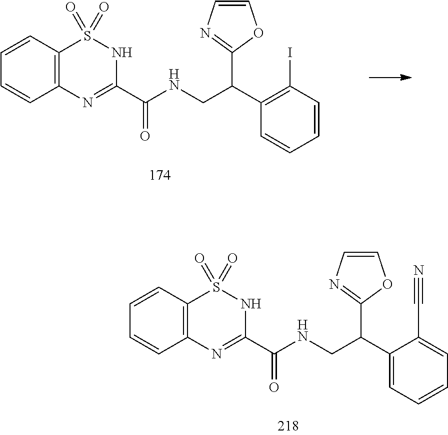 [SMILES: O=C(NCC(c1ncco1)c1ccccc1I)C1=Nc2ccccc2S(=O)(=O)N1 ; N#Cc1ccccc1C(CNC(=O)C1=Nc2ccccc2S(=O)(=O)N1)c1ncco1]

To a solution of N-(2-(2-iodophenyl)-2-(oxazol-2-yl)ethyl)-2H-benzo[e][1,2,4]thiadiazine-3-carboxamide 1,1-dioxide 174 (52 mg, 0.1 mmol) in DMF (2 mL) was added Zn(CN)₂ (24 mg, 0.2 mL) and Pd(PPh₃)₄ (12 mg, 0.01 mmol) and the mixture was bubbled with N₂ for 10 min. The flask was then sealed and the mixture was heated at 130° C. overnight. The mixture was diluted with EtOAc (50 mL), washed with water (50 mL×3) and the organic layer was concentrated under reduced pressure. The residue was purified by prep. TLC (DCM/MeOH=40/1) to give the title compound (20 mg, 50%) as a white solid. LCMS-C: Rₜ 1.18 min, m/z 422.0 [M+H]⁺. ¹H NMR (400 MHz, DMSO-d₆) δ 12.7 (s, 1H), 9.48 (t, J=6.0 Hz, 1H), 8.09 (d, J=0.9 Hz, 1H), 7.89-7.78 (m, 3H), 7.77-7.67 (m, 2H), 7.60-7.46 (m, 3H), 7.23 (s, 1H), 5.02 (t, J=7.6 Hz, 1H), 4.21-4.10 (m, 1H), 4.03-3.92 (m, 1H).

### Example 219: Methyl 2-(2-(1,1-dioxido-2H-benzo[e][1,2,4]thiadiazine-3-carboxamido)-1-(oxazol-2-yl)ethyl)benzoate 219

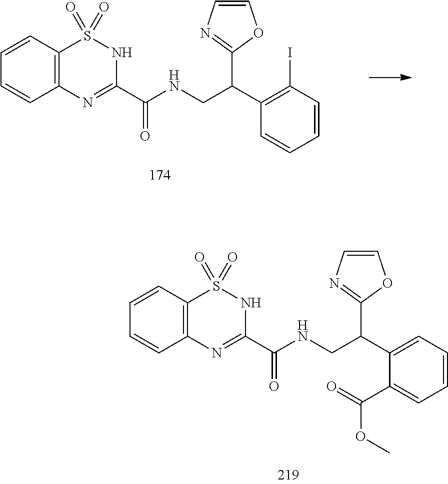 [SMILES: O=C(NCC(c1ncco1)c1ccccc1I)C1=Nc2ccccc2S(=O)(=O)N1 ; COC(=O)c1ccccc1C(CNC(=O)C1=Nc2ccccc2S(=O)(=O)N1)c1ncco1]

To a solution of N-(2-(2-iodophenyl)-2-(oxazol-2-yl)ethyl)-2H-benzo[e][1,2,4]thiadiazine-3-carboxamide 1,1-dioxide 174 (208 mg, 0.4 mmol) in MeOH (40 mL) in a high-pressure reaction vessel was added Et₃N (120 mg, 1.2 mL) and Pd(dppf)Cl₂ (32 mg, 0.04 mmol). The mixture was then heated at 100° C. under a CO atmosphere (0.2 MPa) overnight. The mixture was diluted with water, extracted with EtOAc and the organic layer was dried over Na₂SO₄, filtered and concentrated under reduced pressure. The residue was purified by prep. TLC (DCM/MeOH=20/1) to give the title compound (55 mg, 32%) as a white solid. LCMS-C: Rₜ 1.77 min, m/z 455.0 [M+H]⁺. ¹H NMR (400 MHz, DMSO-d₆) δ 12.6 (s, 1H), 9.21 (t, J=6.0 Hz, 1H), 8.03 (s, 1H), 7.84 (d, J=7.6 Hz, 1H), 7.81-7.75 (m, 2H), 7.75-7.68 (m, 1H), 7.59-7.48 (m, 2H), 7.44-7.38 (m, 1H), 7.33 (dd, J=7.9, 1.2 Hz, 1H), 7.21 (s, 1H), 5.49 (t, J=7.3 Hz, 1H), 4.11-4.01 (m, 1H), 3.91-3.81 (m, 1H), 3.80 (s, 3H).

### Example 220: 7-Iodo-N-(4-methoxy-2-phenylbutyl)-2H-benzo[e][1,2,4]thiadiazine-3-carboxamide 1,1-dioxide 220

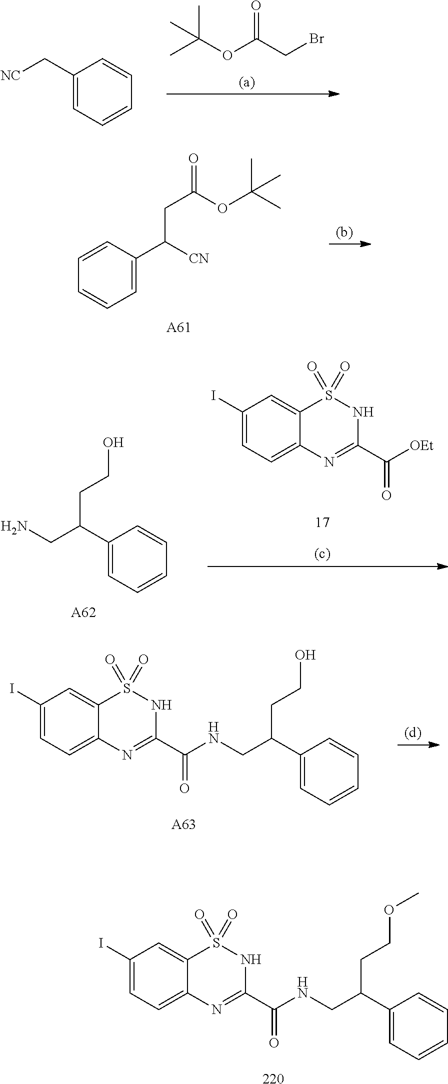

### a) tert-Butyl 3-cyano-3-phenylpropanoate A61

To a solution of 2-phenylacetonitrile (2.34 g, 20 mmol) in dry THF (60 mL) at −78° C. under N₂ was added LiHMDS (1 M solution in THF, 24 mL, 24 mmol) dropwise. The mixture was stirred at −78° C. for 45 min then added to a solution of tert-butyl 2-bromoacetate (4.68 g, 24 mmol) in dry THF (60 mL) at −78° C. under N₂ and the mixture was stirred at −78° C. overnight. The mixture was diluted with water, extracted with EtOAc (300 mL) and the organic layer was washed with water, dried over Na₂SO₄, filtered and concentrated under reduced pressure. The residue was purified by silica gel chromatography (Pet. ether/EtOAc=20/1) to give the title compound (3.8 g, 80%) as a white solid. LCMS-C: Rₜ 2.30 min, m/z 232.0 [M+H]⁺.

### b) 4-Amino-3-phenylbutan-1-ol A62

To a solution of tert-butyl 3-cyano-3-phenylpropanoate A61 (231 mg, 1 mmol) in THF (10 mL) was added LiAlH₄ (1 M solution in THF, 2.0 mL, 2.0 mmol) and the mixture was stirred at RT for 2 h. The mixture was diluted with water, extracted with EtOAc (100 mL) and the organic layer was washed with water, dried over Na₂SO₄, filtered and concentrated under reduced pressure to give the title compound (115 mg, 60%) as a yellow oil. LCMS-A (ES-API): Rₜ 0.322 min, m/z 166.1 [M+H]⁺.

### c) N-(4-Hydroxy-2-phenylbutyl)-7-iodo-2H-benzo[e][1,2,4]thiadiazine-3-carboxamide 1,1-dioxide A63

A suspension of 4-amino-3-phenylbutan-1-ol A62 (115 mg, 0.7 mmol), ethyl 7-iodo-2H-benzo[e][1,2,4]thiadiazine-3-carboxylate 1,1-dioxide I7 (266 mg, 0.7 mmol) and Et₃N (200 mg, 2 mmol) in EtOH (9 mL) was heated at 110° C. in a sealed tube overnight. The mixture was concentrated under reduced pressure and the residue was purified by prep. TLC (DCM/MeOH=20/1) to give the title compound (75 mg, 20%) as a yellow solid. LCMS-C: Rₜ 1.97 min, m/z 499.9 [M+H]⁺.

### d) 7-Iodo-N-(4-methoxy-2-phenylbutyl)-2H-benzo[e][1,2,4]thiadiazine-3-carboxamide 1,1-dioxide 220

To a solution of N-(4-hydroxy-2-phenylbutyl)-7-iodo-2H-benzo[e][1,2,4]thiadiazine-3-carboxamide 1,1-dioxide A63 (75 mg, 0.15 mmol) in CH₃CN (10 mL) was added Ag₂O (174 mg, 0.75 mmol) and iodomethane (213 mg, 1.5 mmol) and the mixture was stirred at RT under N₂ overnight. The mixture was concentrated under reduced pressure and the residue was purified by prep. TLC (DCM/MeOH=20/1) to give the title compound (45 mg, 60%) as a white solid. LCMS-C: Rₜ 2.27 min, m/z 513.9 [M+H]⁺. ¹H NMR (400 MHz, DMSO-d₆) δ 12.7 (s, 1H), 9.20 (t, J=6.0 Hz, 1H), 8.10-8.03 (m, 2H), 7.59 (d, J=8.7 Hz, 1H), 7.34-7.27 (m, 2H), 7.26-7.18 (m, 3H), 3.46 (t, J=6.8 Hz, 2H), 3.20-3.15 (m, 1H), 3.14 (s, 3H), 3.12-3.05 (m, 2H), 2.02-1.90 (m, 1H), 1.79-1.66 (m, 1H).

### Example 221: 7-Chloro-N-(2-(3-hydroxy-5-methylphenyl)-2-(oxazol-2-yl)ethyl)-2H-benzo[e][1,2,4]thiadiazine-3-carboxamide 1,1-dioxide 221

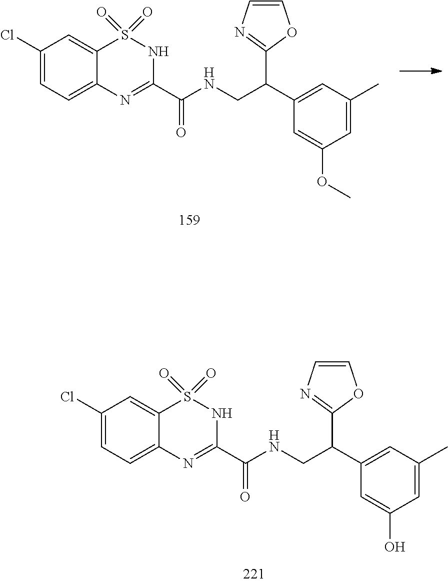 [SMILES: COc1cc(C)cc(C(CNC(=O)C2=Nc3ccc(Cl)cc3S(=O)(=O)N2)c2ncco2)c1 ; Cc1cc(O)cc(C(CNC(=O)C2=Nc3ccc(Cl)cc3S(=O)(=O)N2)c2ncco2)c1]

To a solution of 7-chloro-N-(2-(3-methoxy-5-methylphenyl)-2-(oxazol-2-yl)ethyl)-2H-benzo[e][1,2,4]thiadiazine-3-carboxamide 1,1-dioxide 159 (50 mg, 0.11 mmol) in DCM (5 mL) was added BBr₃ (1 M solution in DCM, 0.33 mL, 0.33 mmol) and the mixture was stirred at RT overnight. The mixture was diluted with water, extracted with diethyl ether and the combined organic extracts were washed with brine, dried over Na₂SO₄, filtered and concentrated under reduced pressure. The residue was purified by prep. HPLC to give the title compound (7 mg, 15%) as a white solid. LCMS-C: Rₜ 1.97 min; m/z 460.9 [M+H]⁺. ¹H NMR (400 MHz, DMSO-d₆) δ 12.8 (s, 1H), 9.32 (s, 1H), 9.24 (t, J=5.9 Hz, 1H), 8.03 (s, 1H), 7.91 (d, J=1.9 Hz, 1H), 7.84-7.77 (m, 2H), 7.19 (s, 1H), 6.50 (s, 1H), 6.49-6.44 (m, 2H), 4.51 (t, J=7.5 Hz, 1H), 4.01-3.92 (m, 1H), 3.83-3.74 (m, 1H), 2.17 (s, 3H).

### Example 222: N-(2-(3-Hydroxy-5-methylphenyl)-2-(oxazol-2-yl)ethyl)-7-iodo-2H-benzo[e][1,2,4]thiadiazine-3-carboxamide 1,1-dioxide 222

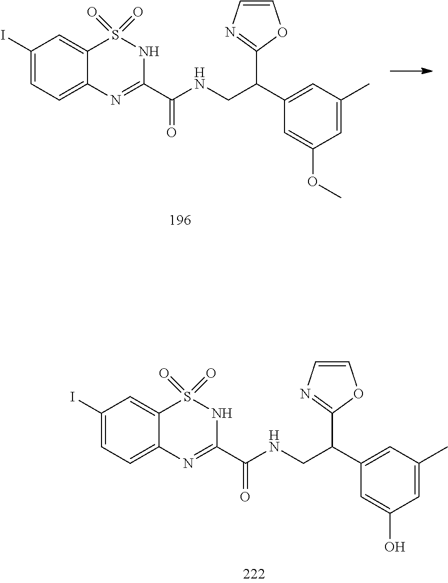 [SMILES: COc1cc(C)cc(C(CNC(=O)C2=Nc3ccc(I)cc3S(=O)(=O)N2)c2ncco2)c1 ; Cc1cc(O)cc(C(CNC(=O)C2=Nc3ccc(I)cc3S(=O)(=O)N2)c2ncco2)c1]

To a solution of 7-iodo-N-(2-(3-methoxy-5-methylphenyl)-2-(oxazol-2-yl)ethyl)-2H-benzo[e][1,2,4]thiadiazine-3-carboxamide 1,1-dioxide 196 (50 mg, 0.09 mmol) in DCM (5 mL) was added BBr₃ (1 M solution in DCM, 0.27 mL, 0.27 mmol) and the mixture was stirred at RT overnight. The mixture was diluted with water, extracted with EtOAc and the combined organic extracts were washed with brine, dried over Na₂SO₄, filtered and concentrated under reduced pressure. The residue was purified by prep. HPLC to give the title compound (1 mg, 3%) as a white solid. LCMS-C: Rₜ 2.07 min; m/z 552.9 [M+H]⁺. ¹H NMR (400 MHz, Methanol-d₄) δ 8.13 (d, J=2.0 Hz, 1H), 7.96 (dd, J=8.7, 2.0 Hz, 1H), 7.86 (d, J=0.9 Hz, 1H), 7.35 (d, J=8.7 Hz, 1H), 7.17 (d, J=0.9 Hz, 1H), 6.61-6.59 (m, 1H), 6.53 (dd, J=10.2, 2.0 Hz, 2H), 4.51-4.46 (m, 1H), 4.07-3.99 (m, 1H), 3.96-3.89 (m, 1H), 2.24 (s, 3H).

### Examples 223 and 224: N-(2-(3-Chlorophenyl)-2-(oxazol-2-yl)ethyl)-7-methoxy-2H-benzo[e][1,2,4]thiadiazine-3-carboxamide 1,1-dioxide 223 and N-(2-(3-chlorophenyl)-2-(oxazol-2-yl)ethyl)-7-hydroxy-2H-benzo[e][1,2,4]thiadiazine-3-carboxamide 1,1-dioxide 224

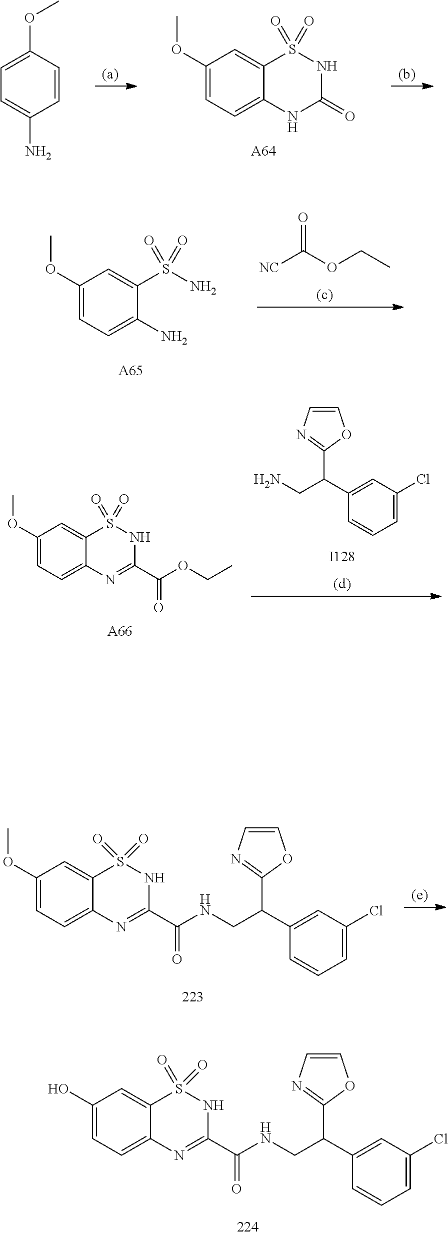

### a) 7-Methoxy-2H-benzo[e][1,2,4]thiadiazin-3(4H)-one 1,1-dioxide A64

To a solution of sulfurisocyanatidic chloride (1.38 g, 9.76 mmol) in nitroethane (8 mL) at −40° C. was added a solution of 4-methoxyaniline (1.0 g, 8.13 mmol) in nitroethane (2 mL) dropwise and the mixture was stirred for 5 min. AlCl₃ (1.08 g, 8.13 mmol) was then added and the mixture was quickly heated to 110° C. and maintained at that temperature for 20 min. The mixture was then poured onto ice and the resulting precipitate was collected by filtration, washed with water and dried under reduced pressure to give the title compound (1.1 g, 60%) as a red solid. LCMS-C: Rₜ 0.32 min; m/z 228.9 [M+H]⁺.

### b) 2-Amino-5-methoxybenzenesulfonamide A65

A mixture of 7-methoxy-2H-benzo[e][1,2,4]thiadiazin-3(4H)-one 1,1-dioxide A64 (600 mg, 2.63 mmol) and 50% (v/v) aqueous H₂SO₄ (20 mL) was heated at 130° C. until a homogeneous solution formed. The mixture was poured onto ice, neutralised and extracted with EtOAc. The organic extract was concentrated under reduced pressure to give the title compound (432 mg, 64%) as a red solid. LCMS-C: Rₜ 0.29 min; m/z 203.0 [M+H]⁺.

### c) Ethyl 7-methoxy-2H-benzo[e][1,2,4]thiadiazine-3-carboxylate 1,1-dioxide A66

A mixture of 2-amino-5-methoxybenzenesulfonamide A65 (432 mg, 2.14 mmol) and ethyl carbonocyanidate (2.12 g, 21.4 mmol) in AcOH (20 mL)/conc. aqueous HCl (0.5 mL) was heated at 85° C. for 4 h. Water was added and the mixture was extracted with EtOAc. The combined organic extracts were washed with brine, dried over Na₂SO₄, filtered and concentrated under reduced pressure to give the title compound (150 mg, 23%) as a white solid. LCMS-C: Rₜ 0.51 min; m/z 284.9 [M+H]⁺.

### d) N-(2-(3-Chlorophenyl)-2-(oxazol-2-yl)ethyl)-7-methoxy-2H-benzo[e][1,2,4]thiadiazine-3-carboxamide 1,1-dioxide 223

A mixture of ethyl 7-methoxy-2H-benzo[e][1,2,4]thiadiazine-3-carboxylate 1,1-dioxide A66 (50 mg, 0.18 mmol), 2-(3-chlorophenyl)-2-(oxazol-2-yl)ethanamine I128 (49 mg, 0.22 mmol) and Et₃N (55 mg, 0.54 mmol) in MeOH (3 mL) was heated at 110° C. in a sealed tube for 3 h. The mixture was allowed to cool to RT, diluted with water and extracted with EtOAc. The combined organic extracts were washed with brine, dried over

Na₂SO₄, filtered and concentrated under reduced pressure. The residue was purified by prep. TLC (DCM/MeOH=20/1) to give the title compound (5.3 mg, 6%) as a white solid. LCMS-C: Rₜ 2.22 min; m/z 460.9 [M+H]⁺. ¹H NMR (400 MHz, DMSO-d₆) δ 12.6 (s, 1H), 9.27 (t, J=6.1 Hz, 1H), 8.07 (d, J=0.9 Hz, 1H), 7.76 (d, J=9.1 Hz, 1H), 7.40-7.31 (m, 4H), 7.28-7.21 (m, 3H), 4.69 (t, J=7.5 Hz, 1H), 4.05-3.86 (m, 2H), 3.85 (s, 3H).

### e) N-(2-(3-Chlorophenyl)-2-(oxazol-2-yl)ethyl)-7-hydroxy-2H-benzo[e][1,2,4]thiadiazine-3-carboxamide 1,1-dioxide 224

To a solution of N-(2-(3-chlorophenyl)-2-(oxazol-2-yl)ethyl)-7-methoxy-2H-benzo[e][1,2,4]thiadiazine-3-carboxamide 1,1-dioxide 223 (15 mg, 0.03 mmol) in DCM (5 mL) was added BBr₃ (1 M solution in DCM, 1.5 mL, 1.5 mmol) and the mixture was stirred at RT for 48 h. The mixture was diluted with water (5 mL), extracted with EtOAc and the combined organic extracts were washed with brine, dried over Na₂SO₄, filtered and concentrated under reduced pressure. The residue was purified by prep. TLC (DCM/MeOH=20/1) to give the title compound (3.1 mg, 23%) as a white solid. LCMS-C: Rₜ 1.98 min; m/z 446.9 [M+H]⁺. ¹H NMR (400 MHz, DMSO-d₆) δ 12.5 (s, 1H), 10.4 (s, 1H), 9.25 (t, J=6.2 Hz, 1H), 8.07 (s, 1H), 7.67 (d, J=9.0 Hz, 1H), 7.42-7.32 (m, 3H), 7.29-7.20 (m, 2H), 7.19-7.12 (m, 1H), 7.08 (d, J=2.7 Hz, 1H), 4.69 (t, J=7.5 Hz, 1H), 4.03-3.84 (m, 2H).

### Examples 225 and 226: 7-Chloro-N-(3-methoxy-2-phenylpropyl)-2H-benzo[e][1,2,4]thiadiazine-3-carboxamide 1,1-dioxide 225 and 7-chloro-N-(3-methoxy-2-phenylpropyl)-2H-benzo[e][1,2,4]thiadiazine-3-carboxamide 1,1-dioxide 226

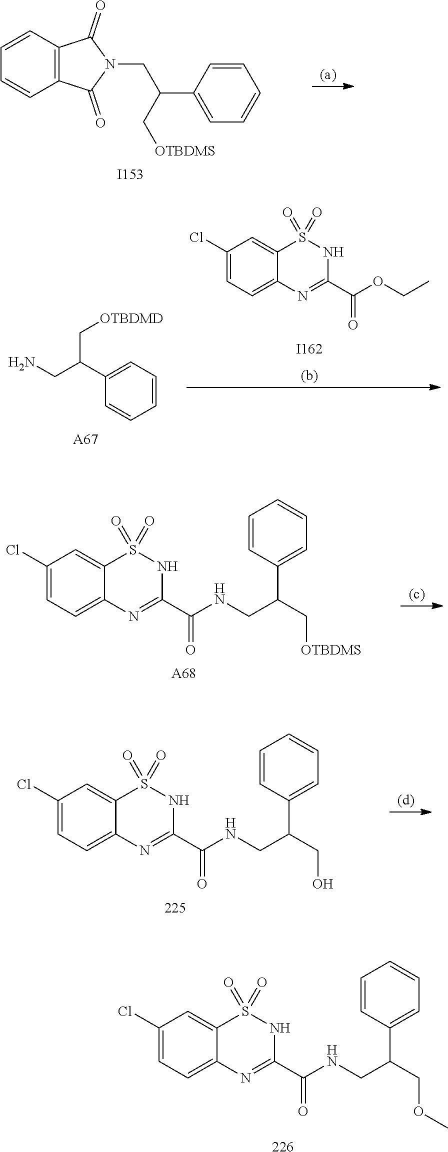

### a) 3-((tert-Butyldimethylsilyl)oxy)-2-phenylpropan-1-amine A67

A solution of 2-(3-((tert-butyldimethylsilyl)oxy)-2-phenylpropyl)isoindoline-1,3-dione I153 (1.0 g, 2.53 mmol) and hydrazine monohydrate (380 mg, 7.58 mmol) in EtOH (50 mL) was heated at 80° C. under N₂ for 3 h. The mixture was filtered and the filter cake was washed with EtOH. The filtrate was concentrated under reduced pressure to give the title compound (0.57 g, 85%) as a yellow oil. LCMS-C: Rₜ 2.85 min; m/z 265.8 [M+H]⁺.

### b) N-(3-((tert-Butyldimethylsilyl)oxy)-2-phenylpropyl)-7-chloro-2H-benzo[e][1,2,4]thiadiazine-3-carboxamide 1,1-dioxide A68

A solution of 3-((tert-butyldimethylsilyl)oxy)-2-phenylpropan-1-amine A67 (200 mg, 0.75 mmol), ethyl 7-chloro-2H-benzo[e][1,2,4]thiadiazine-3-carboxylate 1,1-dioxide I162 (261 mg, 0.90 mmol) and Et₃N (228 mg, 2.25 mmol) in ethanol (15 mL) was heated at 110° C. in a sealed tube for 24 h. The mixture was allowed to cool to RT, diluted with water and extracted with EtOAc. The organic extract was dried over Na₂SO₄, filtered and concentrated under reduced pressure. The residue was purified by silica gel chromatography (DCM/MeOH=20/1) to give the title compound (403 mg, >100%) as white solid, which was used in the next step without further purification. LCMS-C: Rₜ 2.74 min; m/z 508.0 [M+H]⁺. ¹H NMR (400 MHz, DMSO-d₆) δ 12.8 (s, 1H), 9.14 (s, 1H), 7.89 (s, 1H), 7.86-7.75 (m, 2H), 7.36-7.18 (m, 5H), 3.82-3.69 (m, 2H), 3.69-3.53 (m, 2H), 3.25-3.15 (m, 1H), 0.80 (s, 9H), −0.07 (s, 3H), −0.08 (s, 3H).

### c) 7-Chloro-N-(3-hydroxy-2-phenylpropyl)-2H-benzo[e][1,2,4]thiadiazine-3-carboxamide 1,1-dioxide 225

A mixture of N-(3-((tert-butyldimethylsilyl)oxy)-2-phenylpropyl)-7-chloro-2H-benzo[e][1,2,4]thiadiazine-3-carboxamide 1,1-dioxide A68 (383.6 mg, 0.755 mmol) and TBAF (1 M solution in THF, 3.78 mL, 3.78 mmol) in THF (15 mL) was stirred at RT overnight. The mixture was diluted with water, extracted with EtOAc and the organic layer was dried over Na₂SO₄, filtered and concentrated under reduced pressure. The residue was purified by silica gel chromatography (DCM/MeOH=20/1) to give the title compound (140 mg, 47%) as a white solid. LCMS-C: Rₜ 1.71 min; m/z 393.9 [M+H]⁺. ¹H NMR (400 MHz, DMSO-d₆) δ 12.8 (s, 1H), 9.15 (t, J=6.0 Hz, 1H), 7.91 (d, J=2.1 Hz, 1H), 7.87-7.77 (m, 2H), 7.34-7.17 (m, 5H), 4.81 (br s, 1H), 3.68-3.55 (m, 4H), 3.19-3.08 (m, 1H).

### d) 7-Chloro-N-(3-methoxy-2-phenylpropyl)-2H-benzo[e][1,2,4]thiadiazine-3-carboxamide 1,1-dioxide 226

A mixture of 7-chloro-N-(3-hydroxy-2-phenylpropyl)-2H-benzo[e][1,2,4]thiadiazine-3-carboxamide 1,1-dioxide 225 (90.0 mg, 0.23 mmol), Ag₂O (266 mg, 1.15 mmol) and iodomethane (326 mg, 2.3 mmol) in CH₃CN (10 mL) was stirred at RT for 4 days. The mixture was diluted with water, extracted with EtOAc and the organic layer was dried over Na₂SO₄, filtered and concentrated under reduced pressure. The residue was purified by silica gel chromatography (DCM/MeOH=20/1) to give the title compound (8 mg, 9%) as a white solid. LCMS-C: Rₜ 2.20 min; m/z 407.9 [M+H]⁺. ¹H NMR (400 MHz, DMSO-d₆) δ 12.8 (s, 1H), 9.14 (s, 1H), 7.89 (s, 1H), 7.79 (s, 2H), 7.42-7.13 (m, 5H), 3.67-3.46 (m, 4H), 3.30-3.26 (m, 1H), 3.23 (s, 3H).

### Example 227: N-(2-(3-Cyanophenyl)-2-(oxazol-2-yl)ethyl)-2H-benzo[e][1,2,4]thiadiazine-3-carboxamide 1,1-dioxide 227

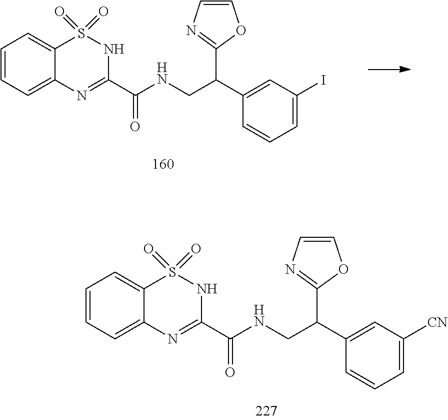 [SMILES: O=C(NCC(c1cccc(I)c1)c1ncco1)C1=Nc2ccccc2S(=O)(=O)N1 ; N#Cc1cccc(C(CNC(=O)C2=Nc3ccccc3S(=O)(=O)N2)c2ncco2)c1]

To a solution of N-(2-(3-iodophenyl)-2-(oxazol-2-yl)ethyl)-2H-benzo[e][1,2,4]thiadiazine-3-carboxamide 1,1-dioxide 160 (52 mg, 0.1 mmol) in DMF (2 mL) was added Pd(PPh₃)₄ (12 mg, 0.01 mmol) and Zn(CN)₂ (24 mg, 0.2 mmol) and the mixture was heated at 120° C. overnight. The mixture was diluted with water, extracted with EtOAc and the combined organic extracts were concentrated under reduced pressure. The residue was purified by prep. TLC (DCM/MeOH=20/1) to give the title compound (20 mg, 47%) as a white solid. LCMS-C: Rₜ 1.28 min; m/z 421.9 [M+H]⁺. ¹H NMR (400 MHz, DMSO-d₆) δ 8.26 (br s, 1H), 7.99-7.75 (m, 3H), 7.75-7.53 (m, 4H), 7.53-7.40 (m, 2H), 7.29-7.21 (m, 1H), 4.82-4.59 (m, 1H), 4.31-3.78 (m, 2H).

### Example 228: N-(2-(2-Hydroxyphenyl)-2-(oxazol-2-yl)ethyl)-7-iodo-2H-benzo[e][1,2,4]thiadiazine-3-carboxamide 1,1-dioxide 228

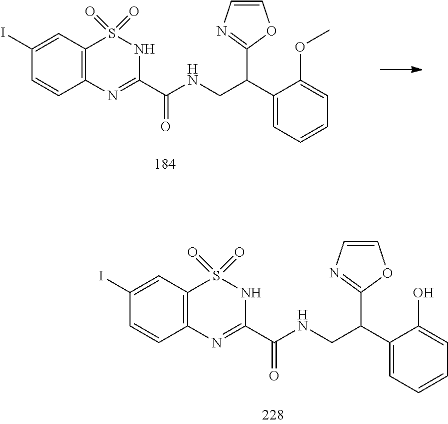 [SMILES: COc1ccccc1C(CNC(=O)C1=Nc2ccc(I)cc2S(=O)(=O)N1)c1ncco1 ; O=C(NCC(c1ncco1)c1ccccc1O)C1=Nc2ccc(I)cc2S(=O)(=O)N1]

To a solution of 7-iodo-N-(2-(2-methoxyphenyl)-2-(oxazol-2-yl)ethyl)-2H-benzo[e][1,2,4]thiadiazine-3-carboxamide 1,1-dioxide 184 (50 mg, 0.09 mmol) in DCM (5 mL) at 0° C. was added BBr₃ (1 M solution in DCM, 0.27 mL, 0.27 mmol) and the mixture was stirred at RT overnight. The reaction was quenched with brine (10 mL) and the mixture was diluted with water (20 mL) and extracted with DCM containing a small amount of MeOH (30 mL×3). The combined organic extracts were washed with brine (30 mL), dried over Na₂SO₄, filtered and concentrated under reduced pressure. The residue was purified by prep. TLC (DCM/MeOH=20/1) to give the title compound (13 mg, 27%) as a white solid. LCMS-C: Rₜ 2.04 min; m/z 538.9 [M+H]⁺. ¹H NMR (400 MHz, DMSO-d₆) δ 12.7 (s, 1H), 9.66 (s, 1H), 9.20 (t, J=5.9 Hz, 1H), 8.11-8.03 (m, 2H), 7.99 (d, J=0.9 Hz, 1H), 7.58 (d, J=8.7 Hz, 1H), 7.17 (d, J=0.8 Hz, 1H), 7.11-7.00 (m, 2H), 6.82 (dd, J=8.1, 1.2 Hz, 1H), 6.78-6.71 (m, 1H), 4.95 (t, J=7.4 Hz, 1H), 4.07-3.96 (m, 1H), 3.84-3.74 (m, 1H).

### Example 155: 2-(2-(7-Iodo-1,1-dioxido-2H-benzo[e][1,2,4]thiadiazine-3-carboxamido)ethyl)benzoic Acid 155

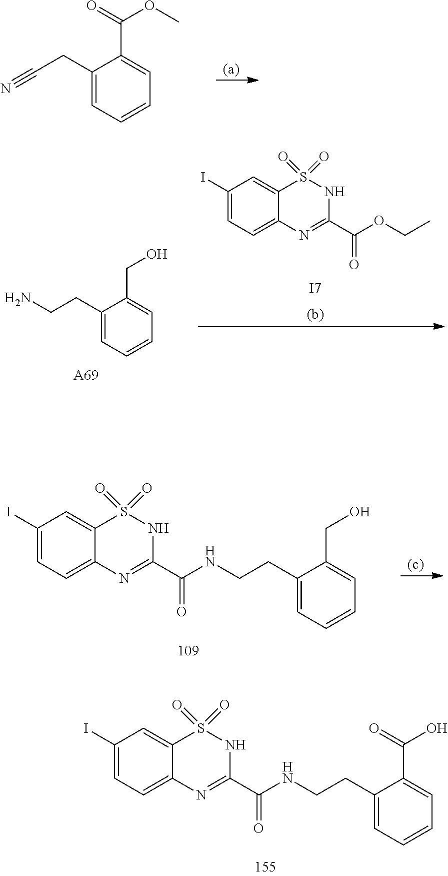

### a) (2-(2-Aminoethyl)phenyl)methanol A69

To a solution of methyl 2-(cyanomethyl)benzoate (3.0 g, 17.1 mmol) in THF (50 mL) was added BH₃.THF (1 M solution in THF, 51.0 mL, 51.0 mmol) and the mixture was heated at 70° C. under N₂ overnight. The mixture was adjusted to pH 5 with 1 M aqueous HCl, diluted with water (20 mL) and washed with EtOAc (30 mL×3). The aqueous phase was adjusted to pH 9 with 1 M aqueous NaOH and extracted with EtOAc (30 mL×3). The combined organic extracts were concentrated under reduced pressure to give the title compound (1.5 g, 57%) as a yellow oil. LCMS-C: Rₜ 0.39; m/z 152.1 [M+H]⁺.

### b) N-(2-(Hydroxymethyl)phenethyl)-7-iodo-2H-benzo[e][1,2,4]thiadiazine-3-carboxamide 1,1-dioxide 109

The following procedure was performed three times: A solution of (2-(2-aminoethyl)phenyl)methanol A69 (300 mg, 1.98 mmol), ethyl 7-iodo-2H-benzo[e][1,2,4]thiadiazine-3-carboxylate 1,1-dioxide I7 (753 mg, 1.98 mmol) and Et₃N (600 mg, 7.84 mmol) in ethanol (10 mL) was heated at 150° C. in a sealed tube for 3 h. The mixture was allowed to cool to RT and concentrated under reduced pressure. The crude product of the three reactions were combined and purified by silica gel chromatography (DCM/MeOH=100/1 to 20/1) to give the title compound (520 mg, 18%) as a white solid. LCMS-D: Rₜ 0.34 min; m/z 486.1 [M+H]⁺.

### c) 2-(2-(7-Iodo-1,1-dioxido-2H-benzo[e][1,2,4]thiadiazine-3-carboxamido)ethyl)benzoic Acid 155

To a solution of N-(2-(hydroxymethyl)phenethyl)-7-iodo-2H-benzo[e][1,2,4]thiadiazine-3-carboxamide 1,1-dioxide 109 (200 mg, 0.4 mmol) in acetone (10 mL) was added Jones reagent (10 mL) and the mixture was heated at 40° C. overnight. The mixture was concentrated under reduced pressure and the residue was diluted with water. The solids were collected by filtration and washed with diethyl ether to give the title compound (115 mg, 55%) as a white solid. LCMS-D: Rₜ 2.64 min; m/z 522.0 [M+Na]⁺. ¹H NMR (400 MHz, DMSO-d₆) δ 12.8 (br s, 1H), 9.43-9.17 (m, 1H), 8.19-7.97 (m, 2H), 7.84 (t, J=8.1 Hz, 1H), 7.69-7.17 (m, 4H), 3.60-3.48 (m, 2H), 3.26-3.19 (m, 2H).

### Example 230: N-(2-Carbamoylphenethyl)-7-iodo-2H-benzo[e][1,2,4]thiadiazine-3-carboxamide 1,1-dioxide 230

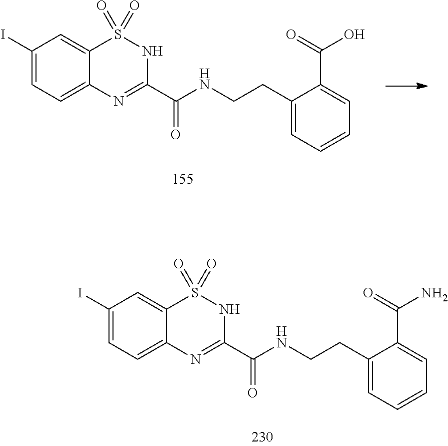 [SMILES: O=C(NCCc1ccccc1C(=O)O)C1=Nc2ccc(I)cc2S(=O)(=O)N1 ; NC(=O)c1ccccc1CCNC(=O)C1=Nc2ccc(I)cc2S(=O)(=O)N1]

To a solution of 2-(2-(7-iodo-1,1-dioxido-2H-benzo[e][1,2,4]thiadiazine-3-carboxamido)ethyl)benzoic acid 155 (50 mg, 0.1 mmol), EDCI (23 mg, 0.12 mmol), DIPEA (39 mg, 0.3 mmol) and HOBt (16 mg, 0.12 mmol) in 1,4-dioxane (5 mL) was added NH₄Cl (11 mg, 0.2 mmol) and the mixture was stirred at RT overnight. The mixture was diluted with water (15 mL), adjusted to pH 5 with 1 M aqueous HCl and extracted with EtOAc (50 mL×3). The combined organic extracts were washed with brine, dried over Na₂SO₄, filtered and concentrated under reduced pressure. The residue was purified by prep. TLC (DCM/MeOH=20/1) to give the title compound (3 mg, 6%) as a grey solid. LCMS-D: Rₜ 2.11 min; m/z 499.0 [M+H]⁺. ¹H NMR (400 MHz, DMSO-d₆) δ 12.6 (br s, 1H), 9.34 (br s, 1H), 8.08-7.98 (m, 2H), 7.79 (s, 1H), 7.57-7.49 (m, 1H), 7.43 (s, 1H), 7.41-7.29 (m, 3H), 7.29-7.22 (m, 1H), 3.55-3.50 (m, 2H), 3.01 (t, J=7.2 Hz, 2H).

### Example 231: N-(2-(2-(difluoromethoxy)phenyl)-2,2-difluoroethyl)-7-iodo-2H-benzo[e][1,2,4]thiadiazine-3-carboxamide 1,1-dioxide 231

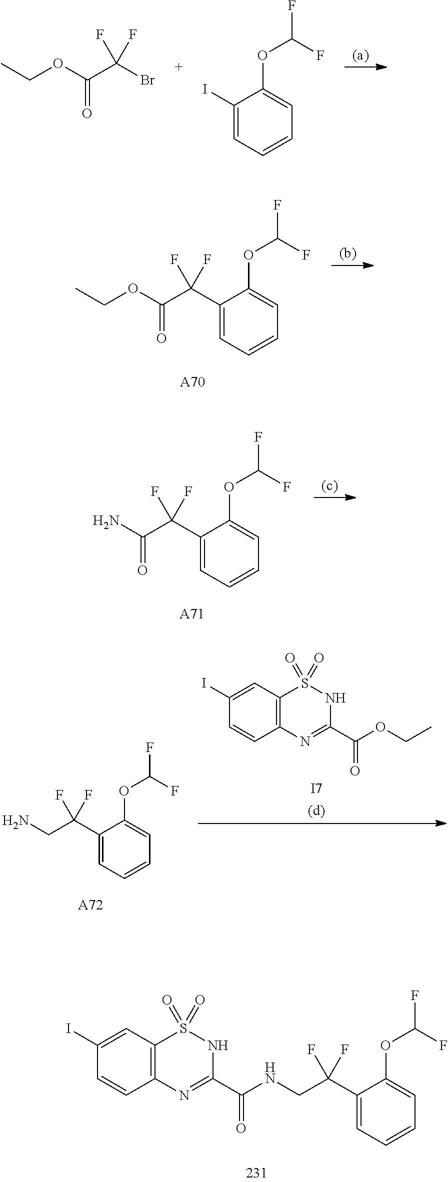

### a) Ethyl 2-(2-(difluoromethoxy)phenyl)-2,2-difluoroacetate A70

To activate Cu powder: Copper powder was stirred vigorously with 1M aqueous HCl (10 mL) for 10 min at RT, then filtered. The process was sequentially repeated with water (10 mL), MeOH (10 mL) and acetone (10 mL). The final filtered material was dried under vacuum for 30 min then used immediately in the reaction.

DMSO (18.5 mL) was added to a nitrogen flushed flask containing activated copper (1.2 g, 19 mmol). 1-(Difluoromethoxy)-2-iodo-benzene (1.1 mL, 7.4 mmol) was added, followed by ethyl bromodifluoroacetate (0.95 mL, 7.4 mmol) and the reaction was heated to 60° C. and stirred overnight. The mixture was cooled and filtered through a pad of Celite® and the Celite® was washed with diethyl ether (100 mL). The green solution was washed with saturated aqueous NH₄Cl (100 mL×2). The now orange organic layer was washed with brine (100 mL), dried (Na₂SO₄) and concentrated in vacuo. The material was purified by column chromatography (Grace Biotage 40 g SiO₂, 0-50% EtOAc in petroleum benzine 40-60° C.) to give the title compound (1.5 g, 77% yield) as a clear oil. ¹H NMR (400 MHz, Chloroform-d) δ 7.74 (dd, J=7.9, 1.7 Hz, 1H), 7.57-7.48 (m, 1H), 7.38-7.31 (m, 1H), 7.23 (dq, J=8.3, 1.2 Hz, 1H), 6.44 (t, J=73.3 Hz, 1H), 4.35 (q, J=7.1 Hz, 2H), 1.33 (t, J=7.1 Hz, 3H).

### b) 2-(2-(Difluoromethoxy)phenyl)-2,2-difluoroacetamide A71

7 M ammonia in MeOH (20 mL) was added to ethyl 2-(2-(difluoromethoxy)phenyl)-2,2-difluoroacetate A70 (1.5 g, 5.6 mmol) and the solution was stirred at RT for 1 h. The mixture was concentrated in vacuo to give the title compound (1.2 g, 90% yield) as an oil. ¹H NMR (400 MHz, Chloroform-d) δ 7.75 (td, J=7.7, 1.7 Hz, 1H), 7.59-7.48 (m, 1H), 7.41-7.29 (m, 1H), 7.29-7.15 (m, 1H), 6.56 (br s, 1H), 6.44 (t, J=73.5 Hz, 1H), 6.11 (br s, 1H).

### c) 2-(2-(Difluoromethoxy)phenyl)-2,2-difluoroethan-1-amine A72

To 2-(2-(Difluoromethoxy)phenyl)-2,2-difluoroacetamide A71 (1.2 g, 5.1 mmol) in THF (25 mL) at 0° C. was added borane-tetrahydrofuran complex 1.0 M solution in THF (2.4 mL, 2.4 mmol) dropwise. The solution was allowed to warm to RT and stirred overnight. The reaction was cooled to 0° C. and quenched with the slow addition of MeOH until gas evolution ceased (˜25 mL). Conc. HCl was added (˜20 mL) and the reaction allowed to stir for 1 h upon which time the mixture was concentrated to dryness. The crude material was loaded onto a Biotage SCX cartridge (2×10 g) and washed with MeOH (50 mL), then a methanolic ammonia solution (50 mL). The basic washings were concentrated in vacuo to give the title compound (0.14 g, 12% yield) as an orange oil. LCMS-B: rt 2.772 min; m/z 223.9 [M+H]⁺. ¹H NMR (400 MHz, Chloroform-d) δ 7.62 (dd, J=7.6, 1.7 Hz, 1H), 7.52-7.43 (m, 1H), 7.35-7.26 (m, 1H), 7.25-7.21 (m, 1H), 6.46 (t, J=74.0 Hz, 1H), 3.33 (t, J=15.1 Hz, 2H).

### d) N-(2-(2-(Difluoromethoxy)phenyl)-2,2-difluoroethyl)-7-iodo-2H-benzo[e][1,2,4]thiadiazine-3-carboxamide 1,1-dioxide 231

A suspension of 2-(2-(difluoromethoxy)phenyl)-2,2-difluoroethan-1-amine A72 (0.038 g, 0.17 mmol) and ethyl 7-iodo-2H-benzo[e][1,2,4]thiadiazine-3-carboxylate 1,1-dioxide I7 (0.050 g, 0.13 mmol) in EtOH (0.125 mL) was irradiated in a CEM microwave at 100° C. for 2 h. The reaction was cooled and the precipitate filtered, then washed with EtOH (2 mL). The filtrate was concentrated to dryness, then partitioned between EtOH (2 mL) and 1 M aqueous HCl (2 mL). The layers were separated and the organics washed with a further portion of 1 M aqueous HCl (2 mL), brine (2 mL), dried (Na₂SO₄) and concentrated in vacuo. The crude material was purified by column chromatography (Santai Sepa-Flash, 12 g SiO₂, 0-100% EtOAc in petroleum benzine 40-60° C.) with the material eluting at ˜50% EtOAc collected and concentrated in vacuo to give the title compound (0.010 g, 14% yield) as a cream-colored solid. LCMS-B: rt 3.678 min; m/z 555.7 [M−H]⁻. ¹H NMR (400 MHz, DMSO-d₆) δ 12.76 (br s, 1H), 9.47 (br s, 1H), 8.11-7.96 (m, 2H), 7.69-7.47 (m, 3H), 7.33 (t, J=8.1 Hz, 2H), 7.26 (t, J=73.3 Hz, 1H), 4.34-3.91 (m, 2H).

### Example 232: 7-chloro-N-(2-(2-(difluoromethoxy)phenyl)-2,2-difluoroethyl)-2H-benzo[e][1,2,4]thiadiazine-3-carboxamide 1,1-dioxide 232

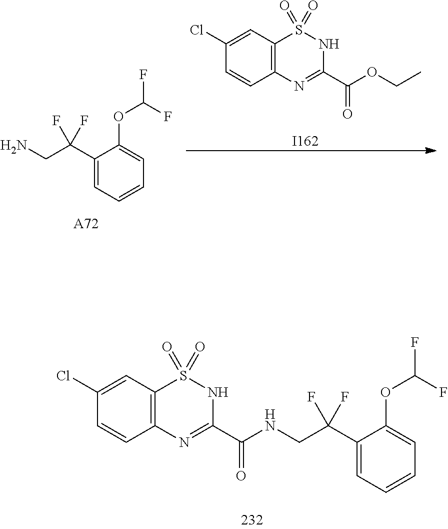 [SMILES: "I162" ; NCC(F)(F)c1ccccc1OC(F)F ; CCOC(=O)C1=Nc2ccc(Cl)cc2S(=O)(=O)N1 ; "A72" ; O=C(NCC(F)(F)c1ccccc1OC(F)F)C1=Nc2ccc(Cl)cc2S(=O)(=O)N1]

A suspension of 2-(2-(difluoromethoxy)phenyl)-2,2-difluoroethan-1-amine A72 (0.048 g, 0.22 mmol) and ethyl 7-chloro-2H-benzo[e][1,2,4]thiadiazine-3-carboxylate 1,1-dioxide I162 (0.048 g, 0.17 mmol) in EtOH (0.2 mL) was irradiated in a CEM microwave at 120° C. for 1 h. The crude material was purified by column chromatography (Santai Sepa-Flash, 12 g SiO₂, 0-100% EtOAc in petroleum benzine 40-60° C.) to give the title compound (0.022 g, 28% yield) as a white solid. LCMS-B: rt 3.857 min; m/z 463.8 [M−H]⁻. ¹H NMR (400 MHz, DMSO-d₆) δ 12.82 (br s, 1H), 9.53 (br s, 1H), 7.92 (s, 1H), 7.81 (s, 2H), 7.68-7.49 (m, 2H), 7.33 (t, J=8.1 Hz, 2H), 7.26 (t, J=73.3 Hz, 1H), 4.10 (td, J=14.2, 6.6 Hz, 2H).

### Example 233: 7-iodo-N-(2-(oxazol-2-yl)-2-(m-tolyl)ethyl)-2H-benzo[e][1,2,4]thiadiazine-3-carboxamide 1,1-dioxide 233

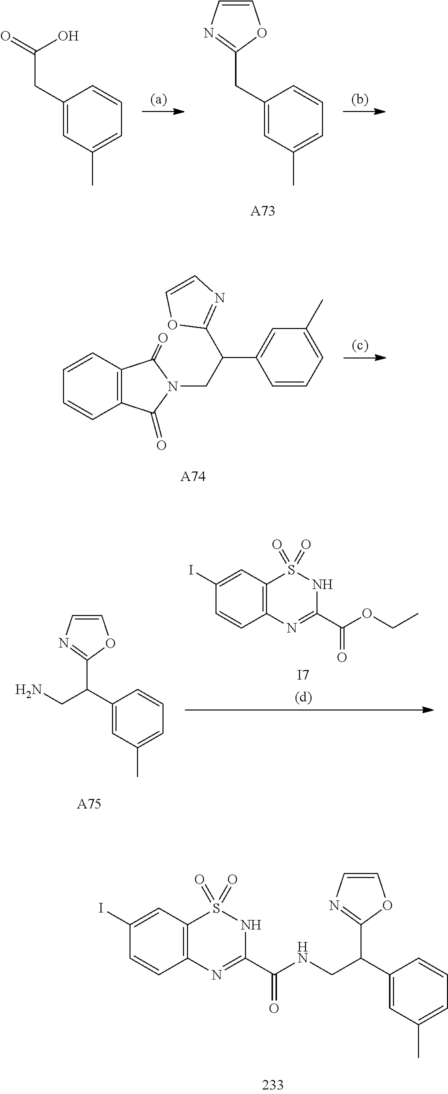

### a) 2-(3-Methylbenzyl)oxazole A73

m-Tolylacetic acid (5.0 g, 33 mmol) was dissolved in thionyl chloride (25 mL) and heated at 80° C. for 3 h. The remaining thionyl chloride was evaporated in vacuo. The residue was dissolved in sulfolane (10 mL), and to this was added 1H-1,2,3-Triazole (2.7 mL, 47 mmol) and K₂CO₃(9.2 g, 67 mmol). The reaction was heated to 150° C. for 30 min, then cooled, added to water (30 mL) and extracted with EtOAc (3×30 mL). The combined organics were washed with brine, dried (Na₂SO₄) and concentrated in vacuo. The crude material was purified by silica gel chromatography (Isolera Biotage 120 g SiO₂, 0-30% EtOAc in petroleum benzine 40-60° C.) to give the title compound (0.58 g, 10% yield) as a clear oil. LCMS-B: rt 3.268 min, m/z 174.0 [M+H]⁺. ¹H NMR (400 MHz, Chloroform-d) δ 7.56 (d, J=0.9 Hz, 1H), 7.22 (td, J=7.6, 0.7 Hz, 1H), 7.13-7.05 (m, 3H), 7.04 (d, J=0.9 Hz, 1H), 4.09 (s, 2H), 2.33 (d, J=0.7 Hz, 3H).

### b) 2-(2-(Oxazol-2-yl)-2-(m-tolyl)ethyl)isoindoline-1,3-dione A74

To a solution of 2-(3-methylbenzyl)oxazole A73 (0.573 g, 3.31 mmol) in anhydrous THF (10 mL) at −78° C. under nitrogen was added lithium bis(trimethylsilyl)amide, 1.0 M solution in hexane (4.96 mL, 4.96 mmol) dropwise. A solution of N-(bromomethyl)phthalimide (1.19 g, 4.96 mmol) in anhydrous THF (8 mL) was then added dropwise and the mixture allowed to warm slowly to room temperature and stirred overnight. The mixture was diluted with a saturated aqueous NH₄Cl solution (50 mL) and water (25 mL), then extracted with DCM (50 mL×3). The combined organic extracts were washed with brine, dried (Na₂SO₄), concentrated in vacuo and purified by column chromatography (Biotage, Grace 40 g SiO₂, 0-60% EtOAc in petroleum benzine 40-60° C.) to give the title compound (0.11 g, 10% yield) as a white solid. LCMS-A: rt 6.117 min; m/z 332.9 [M+H]⁺. ¹H NMR (400 MHz, Chloroform-d) δ 7.63 (dd, J=5.5, 3.0 Hz, 2H), 7.52 (dd, J=5.5, 3.0 Hz, 2H), 7.42 (d, J=1.0 Hz, 1H), 7.05-6.96 (m, 3H), 6.92-6.82 (m, 2H), 4.61 (t, J=8.1 Hz, 1H), 4.25 (dd, J=13.7, 8.1 Hz, 1H), 4.16 (dd, J=13.7, 8.2 Hz, 1H), 2.12 (s, 3H).

### c) 2-(Oxazol-2-yl)-2-(m-tolyl)ethan-1-amine A75

To a suspension of 2-(2-(oxazol-2-yl)-2-(m-tolyl)ethyl)isoindoline-1,3-dione A74 (0.11 g, 0.34 mmol) in EtOH (3 mL), under an atmosphere of nitrogen, was added hydrazine hydrate (0.251 g, 5.01 mmol). This was heated to 80° C. and allowed to stir for 3 h, upon which time the reaction was cooled and the formed precipitate filtered. The solid was washed with cold EtOH (1 mL) and the combined filtrate concentrated in vacuo. The resulting solid was taken up in cold EtOH (1 mL) and filtered. The filtrate was concentrated in vacuo. The resulting semi-solid was once more taken up in cold EtOH (1 mL), the precipitate was filtered and the filtrate concentrated in vacuo to give the title compound (0.045 g, 66% yield) as a yellow oil. LCMS-B: rt 2.741 min; m/z 203.0 [M+H]⁺.

### d) 7-Iodo-N-(2-(oxazol-2-yl)-2-(m-tolyl)ethyl)-2H-benzo[e][1,2,4]thiadiazine-3-carboxamide 1,1-dioxide 233

To a solution of 2-(oxazol-2-yl)-2-(m-tolyl)ethan-1-amine A75 (0.022 g, 0.11 mmol) in EtOH (0.125 mL) was added ethyl 7-iodo-2H-benzo[e][1,2,4]thiadiazine-3-carboxylate 1,1-dioxide I7 (0.034 g, 0.091 mmol). This was irradiated in a CEM microwave at 120° C. for 2 h. The reaction was cooled and the precipitate filtered. The solid was washed with EtOH (2 mL) and air dried to give title compound (0.020 g, 34% yield) as an off-white solid. LCMS-B: rt 3.565 min; m/z 536.6 [M+H]⁺. ¹H NMR (400 MHz, DMSO-d₆) δ 12.72 (br s, 1H), 9.17 (br s, 1H), 8.10-7.92 (m, 3H), 7.57-7.47 (m, 1H), 7.26-7.17 (m, 2H), 7.15-7.00 (m, 3H), 4.61 (t, J=7.5 Hz, 1H), 3.99 (dt, J=13.4, 6.8 Hz, 1H), 3.84 (dt, J=13.3, 6.8 Hz, 1H), 2.27 (s, 3H).

### Example 234: 7-chloro-N-(2-(oxazol-2-yl)-2-(m-tolyl)ethyl)-2H-benzo[e][1,2,4]thiadiazine-3-carboxamide 1,1-dioxide 234

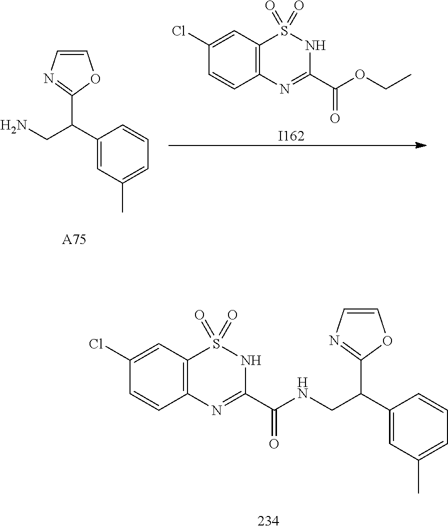 [SMILES: "I162" ; Cc1cccc(C(CN)c2ncco2)c1 ; CCOC(=O)C1=Nc2ccc(Cl)cc2S(=O)(=O)N1 ; "A75" ; Cc1cccc(C(CNC(=O)C2=Nc3ccc(Cl)cc3S(=O)(=O)N2)c2ncco2)c1]

To a solution of 2-(oxazol-2-yl)-2-(m-tolyl)ethan-1-amine A75 (0.020 g, 0.099 mmol) in EtOH (0.125 mL) was added ethyl 7-chloro-2H-benzo[e][1,2,4]thiadiazine-3-carboxylate 1,1-dioxide I162 (0.024 g, 0.082 mmol). The reaction was irradiated in a CEM microwave at 120° C. for 1 h. The reaction was cooled and the precipitate filtered. The solid was washed with EtOH (2 mL) and air dried to give the title compound (0.020 g, 45% yield) as a white solid. LCMS-B: rt 3.616 min; m/z 444.7 [M+H]⁺. ¹H NMR (400 MHz, DMSO-d₆) δ 12.79 (br s, 1H), 9.23 (br s, 1H), 8.04 (d, J=0.9 Hz, 1H), 7.89 (s, 1H), 7.86-7.66 (m, 2H), 7.29-7.16 (m, 2H), 7.15-6.95 (m, 3H), 4.62 (t, J=7.5 Hz, 1H), 4.00 (dt, J=13.3, 6.6 Hz, 1H), 3.85 (dt, J=13.4, 6.8 Hz, 1H), 2.27 (s, 3H).

### Example 235: N-(2-(2-fluorophenyl)-2-(oxazol-2-yl)ethyl)-7-iodo-2H-benzo[e][1,2,4]thiadiazine-3-carboxamide 1,1-dioxide 235

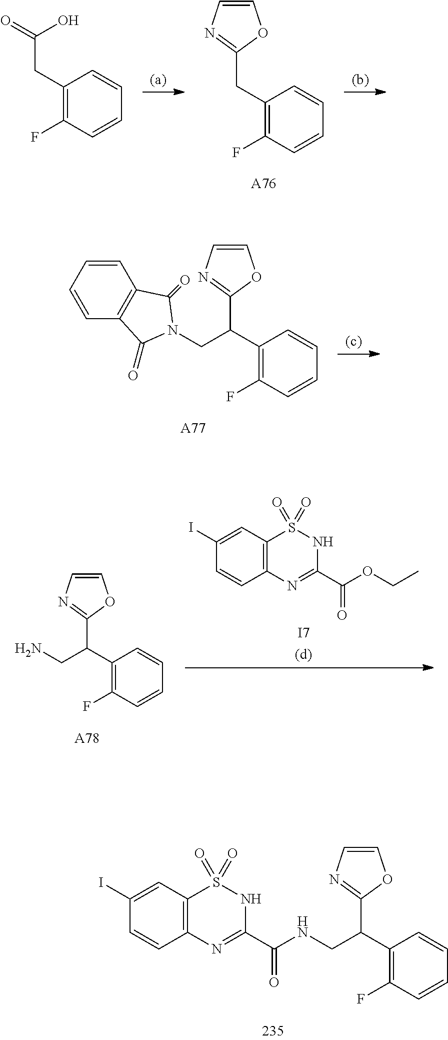

### a) 2-(2-Fluorobenzyl)oxazole A76

2-Fluorophenylacetic acid (3.0 g, 19 mmol) was dissolved in thionyl chloride (15 mL) and heated at 80° C. for 3 h. The remaining thionyl chloride was evaporated in vacuo. The residue was dissolved in sulfolane (10 mL), and to this was added 1H-1,2,3-triazole (1.6 mL, 27 mmol) and K₂CO₃ (5.4 g, 39 mmol). The reaction was heated to 150° C. for 30 min, then cooled, added to water (20 mL) and extracted with EtOAc (3×20 mL). The combined organics were washed with brine, dried (Na₂SO₄) and concentrated in vacuo. The crude material was purified by silica gel chromatography (Isolera Biotage 120 g SiO₂, 0-20% EtOAc in petroleum benzine 40-60° C.) to give the title compound (1.6 g, 47% yield) as a clear oil. LCMS-B: rt 3.322 min, m/z 178.0 [M+H]⁺. ¹H NMR (400 MHz, Chloroform-d) δ 7.57 (d, J=0.9 Hz, 1H), 7.31-7.20 (m, 3H), 7.15-7.02 (m, 3H), 4.17 (s, 2H).

### b) 2-(2-(2-Fluorophenyl)-2-(oxazol-2-yl)ethyl)isoindoline-1,3-dione A77

To a solution of 2-(2-fluorobenzyl)oxazole A76 (1.63 g, 9.21 mmol) in anhydrous THF (30 mL) at −78° C. under nitrogen was added lithium bis(trimethylsilyl)amide, 1.0 M solution in hexane (13.8 mL, 13.8 mmol) dropwise. A solution of N-(bromomethyl)phthalimide (2.87 g, 12.0 mmol) in anhydrous THF (25 mL) was then added dropwise and the mixture allowed to warm slowly to RT and left to stir overnight. The mixture was diluted with a saturated aqueous NH₄Cl solution (100 mL) and water (50 mL), then extracted with DCM (3×100 mL). The combined organic extracts were washed with brine, dried (Na₂SO₄), concentrated in vacuo and purified by column chromatography (Isolera Biotage, Grace 120 g SiO₂, 0-60% EtOAc in petroleum benzine 40-60° C.) to give the title compound (0.91 g, 30% yield) as a white solid. LCMS-B: rt 3.434 min; m/z 336.9 [M+H]⁺. ¹H NMR (400 MHz, Chloroform-d) δ 7.83-7.77 (m, 2H), 7.73-7.67 (m, 2H), 7.60 (d, J=0.9 Hz, 1H), 7.42-7.35 (m, 1H), 7.15-7.07 (m, 2H), 7.02-6.92 (m, 1H), 5.11 (dd, J=8.8, 7.1 Hz, 1H), 4.50-4.33 (m, 2H). One aromatic proton obscured by solvent signal.

### c) 2-(2-Fluorophenyl)-2-(oxazol-2-yl)ethan-1-amine A78

To a suspension of 2-(2-(2-fluorophenyl)-2-(oxazol-2-yl)ethyl)isoindoline-1,3-dione A77 (0.20 g, 0.59 mmol) in EtOH (6 mL), under an atmosphere of nitrogen, was added hydrazine hydrate (0.430 mL, 8.84 mmol). The reaction was heated to 80° C. and allowed to stir for 3 h, upon which time the reaction was cooled and the formed precipitate filtered.

The solid was washed with cold EtOH (2 mL) and the combined filtrate concentrated in vacuo. The resulting solid was taken up in cold EtOH (1 mL) and filtered. The filtrate was concentrated in vacuo. The resulting semi-solid was once more taken up in cold EtOH (1 mL), the precipitate filtered and the filtrate concentrated in vacuo to give the title compound (0.11 g, 90% yield) as an orange oil. LCMS-B: rt 2.718 min; m/z 207.0 [M+H]⁺. ¹H NMR (400 MHz, Chloroform-d) δ 7.60 (d, J=0.9 Hz, 1H), 7.26-7.22 (m, 1H), 7.22-7.14 (m, 1H), 7.13-7.03 (m, 3H), 4.56 (dd, J=7.9, 6.0 Hz, 1H), 3.50-3.40 (m, 1H), 3.25 (dd, J=12.9, 6.0 Hz, 1H).

### d) N-(2-(2-Fluorophenyl)-2-(oxazol-2-yl)ethyl)-7-iodo-2H-benzo[e][1,2,4]thiadiazine-3-carboxamide 1,1-dioxide 235

To a suspension of ethyl 7-iodo-2H-benzo[e][1,2,4]thiadiazine-3-carboxylate 1,1-dioxide I7 (0.040 g, 0.11 mmol) in EtOH (0.125 mL) was added 2-(2-fluorophenyl)-2-(oxazol-2-yl)ethan-1-amine A78 (0.026 g, 0.13 mmol). The reaction was irradiated in a CEM microwave at 120° C. for 3 h. The reaction was cooled and the precipitate filtered. The solid was washed with EtOH (2 mL) and air dried to give the title compound (0.020 g, 35% yield) as a white solid. LCMS-B: rt 3.591 min; m/z 540.6 [M+H]⁺. ¹H NMR (400 MHz, DMSO-d₆) δ 12.74 (s, 1H), 9.38 (t, J=6.0 Hz, 1H), 8.13-7.98 (m, 3H), 7.57 (dd, J=19.2, 8.7 Hz, 2H), 7.35 (tdd, J=8.5, 3.7, 1.5 Hz, 2H), 7.25-7.06 (m, 2H), 4.94 (t, J=7.6 Hz, 1H), 4.06 (ddd, J=12.9, 7.2, 5.7 Hz, 1H), 3.90 (ddd, J=13.2, 8.1, 6.4 Hz, 1H).

### Example 236: 7-chloro-N-(2-(2-fluorophenyl)-2-(oxazol-2-yl)ethyl)-2H-benzo[e][1,2,4]thiadiazine-3-carboxamide 1,1-dioxide 236

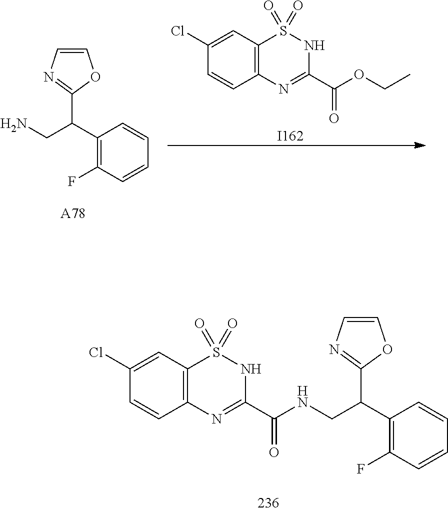

To a suspension of ethyl 7-chloro-2H-benzo[e][1,2,4]thiadiazine-3-carboxylate 1,1-dioxide I162 (0.028 g, 0.097 mmol) in EtOH (0.125 mL) was added 2-(2-fluorophenyl)-2-(oxazol-2-yl)ethan-1-amine A78 (0.024 g, 0.12 mmol). The reaction was irradiated in a CEM microwave at 120° C. for 1 h. The reaction was cooled and the precipitate filtered. The solid was washed with EtOH (1 mL) and air dried to give the title compound (0.015 g, 29% yield) as a white solid. LCMS-B: rt 3.562 min; m/z 448.7 [M+H]⁺. ¹H NMR (400 MHz, DMSO-d₆) δ 12.80 (br s, 1H), 9.34 (br s, 1H), 8.06 (d, J=0.8 Hz, 1H), 7.90 (s, 1H), 7.80 (s, 2H), 7.35 (dddt, J=9.3, 7.4, 3.7, 1.7 Hz, 2H), 7.26-7.12 (m, 3H), 4.94 (t, J=7.5 Hz, 1H), 4.06 (dt, J=13.0, 6.4 Hz, 1H), 3.91 (dt, J=13.6, 7.1 Hz, 1H).

### Example 237: 7-iodo-N-(2-(oxazol-2-yl)-2-(o-tolyl)ethyl)-2H-benzo[e][1,2,4]thiadiazine-3-carboxamide 1,1-dioxide 237

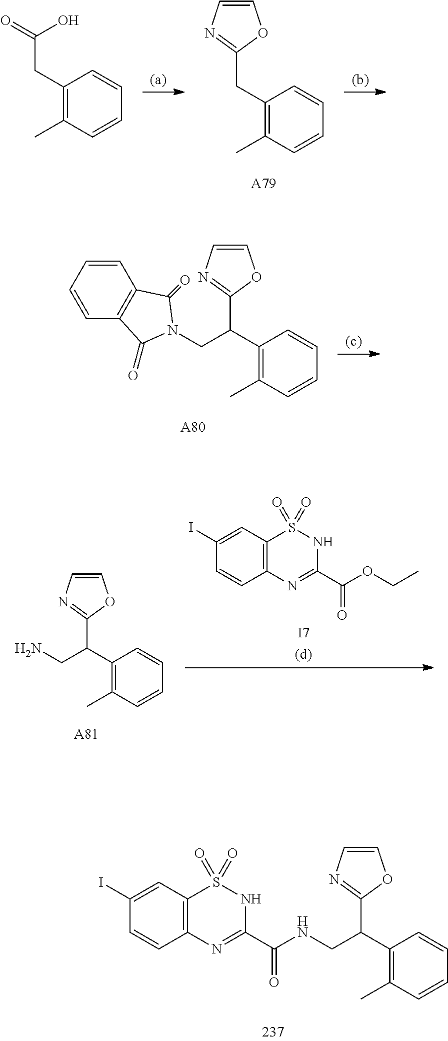

### a) 2-(2-Methylbenzyl)oxazole A79

2-(o-Tolyl)acetic acid (2.0 g, 13 mmol) was dissolved in thionyl chloride (10 mL) and heated at 80° C. for 3 h. The remaining thionyl chloride was evaporated in vacuo. The residue was dissolved in sulfolane (10 mL), and to this was added 1H-1,2,3-triazole (1.08 mL, 18.6 mmol) and K₂CO₃ (3.7 g, 27 mmol). The reaction was heated to 150° C. for 30 min, then cooled, added to water (20 mL) and extracted with EtOAc (3×20 mL). The combined organics were washed with brine, dried (Na₂SO₄) and concentrated in vacuo. The crude material was purified by silica gel chromatography (Isolera Biotage 120 g SiO₂, 0-60% EtOAc in petroleum benzine 40-60° C.) to give the title compound (0.63 g, 27% yield) as a clear oil. LCMS-B: rt 3.185 min, m/z 174.0 [M+H]⁺. ¹H NMR (400 MHz, Chloroform-d) δ 7.55 (d, J=0.9 Hz, 1H), 7.22-7.15 (m, 4H), 7.03 (d, J=0.9 Hz, 1H), 4.12 (s, 2H), 2.34 (s, 3H).

### b) 2-(2-(Oxazol-2-yl)-2-(o-tolyl)ethyl)isoindoline-1,3-dione A80

To a solution of 2-(2-methylbenzyl)oxazole A79 (0.62 g, 3.6 mmol) in anhydrous THF (10 mL) at −78° C. under nitrogen was added lithium bis(trimethylsilyl)amide, 1.0 M solution in hexane (4.68 mL, 4.68 mmol) dropwise. A solution of N-(bromomethyl)phthalimide (1.12 g, 4.68 mmol) in anhydrous THF (8 mL) was then added dropwise and the mixture allowed to warm slowly to room temperature and stirred overnight. The mixture was diluted with a saturated aqueous NH₄Cl solution (50 mL) and water (25 mL), then extracted with DCM (3×50 mL). The combined organic extracts were washed with brine, dried (Na₂SO₄), concentrated in vacuo and purified by column chromatography (Isolera Biotage, Grace 40 g SiO₂, 0-60% EtOAc in petroleum benzine 40-60° C.) to give the title compound (0.070 g, 5.9% yield) as a white solid. LCMS-B: rt 3.414 min; m/z 332.9 [M+H]⁺. ¹H NMR (400 MHz, Chloroform-d) δ 7.79 (dd, J=5.5, 3.0 Hz, 2H), 7.68 (dd, J=5.5, 3.0 Hz, 2H), 7.56 (d, J=0.9 Hz, 1H), 7.47-7.41 (m, 1H), 7.23-7.17 (m, 1H), 7.17-7.12 (m, 2H), 7.02 (d, J=0.8 Hz, 1H), 5.09 (dd, J=8.8, 7.1 Hz, 1H), 4.49 (dd, J=13.7, 8.8 Hz, 1H), 4.25 (dd, J=13.7, 7.2 Hz, 1H), 2.42 (s, 3H).

### c) 2-(Oxazol-2-yl)-2-(o-tolyl)ethan-1-amine A81

To a suspension of 2-(2-(oxazol-2-yl)-2-(o-tolyl)ethyl)isoindoline-1,3-dione A80 (0.067 g, 0.20 mmol) in EtOH (3 mL), under an atmosphere of nitrogen, was added hydrazine hydrate (0.150 g, 3.00 mmol). This was heated to 80° C. and allowed to stir for 3 h, upon which time the reaction was cooled and the formed precipitate filtered. The solid was washed with cold EtOH (1 mL) and the combined filtrates concentrated in vacuo. The resulting solid was taken up in cold EtOH (1 mL) and filtered. The filtrate was concentrated in vacuo. The resulting semi-solid was once more taken up in cold EtOH (1 mL), the precipitate filtered and the filtrate concentrated in vacuo to give the title compound (0.024 g, 59% yield) as a yellow oil. ¹H NMR (400 MHz, Chloroform-d) δ 7.57 (d, J=0.8 Hz, 1H), 7.23-7.17 (m, 1H), 7.18-7.13 (m, 2H), 7.12-7.07 (m, 2H), 4.45 (dd, J=8.4, 5.7 Hz, 1H), 3.46 (dd, J=13.0, 8.4 Hz, 1H), 3.22 (dd, J=13.0, 5.7 Hz, 1H), 2.44 (s, 3H).

### d) 7-Iodo-N-(2-(oxazol-2-yl)-2-(o-tolyl)ethyl)-2H-benzo[e][1,2,4]thiadiazine-3-carboxamide 1,1-dioxide 237

To a solution of 2-(oxazol-2-yl)-2-(o-tolyl)ethan-1-amine A81 (0.022 g, 0.11 mmol) in EtOH (0.125 mL) was added ethyl 7-iodo-2H-benzo[e][1,2,4]thiadiazine-3-carboxylate 1,1-dioxide I7 (0.034 g, 0.089 mmol). The reaction was irradiated in a CEM microwave at 120° C. for 1.5 h, then cooled and the precipitate filtered. The solid was washed with EtOH (2 mL) and air dried to give title compound (0.031 g, 54% yield) as a white solid. LCMS-B: rt 3.431 min; m/z 536.6 [M+H]⁺. ¹H NMR (400 MHz, DMSO-d₆) δ 12.77 (br s, 1H), 9.24 (br s, 1H), 8.15-7.91 (m, 3H), 7.52 (d, J=8.5 Hz, 1H), 7.27-7.10 (m, 5H), 4.91 (t, J=7.5 Hz, 1H), 4.04 (dt, J=14.0, 7.5 Hz, 1H), 3.78 (dt, J=12.8, 6.1 Hz, 1H), 2.40 (s, 3H).

### Example 238: N-(2,2-difluoro-2-phenylethyl)-7-iodo-2H-benzo[e][1,2,4]thiadiazine-3-carboxamide 1,1-dioxide 238

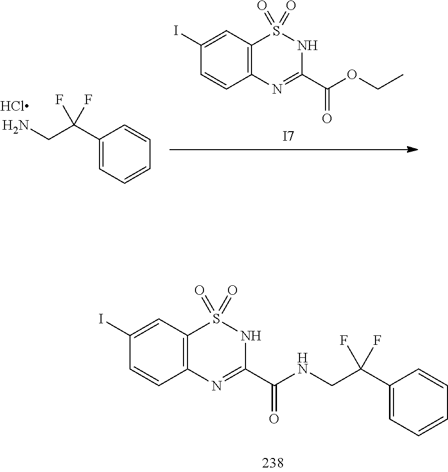

To 2,2-difluoro-2-phenyl-ethanamine hydrochloride (0.031 g, 0.16 mmol) in EtOH (0.125 mL), was added triethylamine (0.022 mL, 0.16 mmol). This was allowed to stir for 10 min at RT upon which time ethyl 7-iodo-2H-benzo[e][1,2,4]thiadiazine-3-carboxylate 1,1-dioxide I7 (0.050 g, 0.13 mmol) was added. The reaction was irradiated in a CEM microwave for 1.5 h at 120° C., then cooled and the precipitate filtered. The solid was washed with cold EtOH (2 mL) and air dried to give the title compound (0.033 g, 51% yield) as a cream solid. LCMS-B: rt 3.344 min; m/z 489.7 [M−H]⁻.

### Example 239: 7-iodo-N-(2-(4-(methoxymethyl)-2H-1,2,3-triazol-2-yl)phenethyl)-2H-benzo[e][1,2,4]thiadiazine-3-carboxamide 1,1-dioxide 239

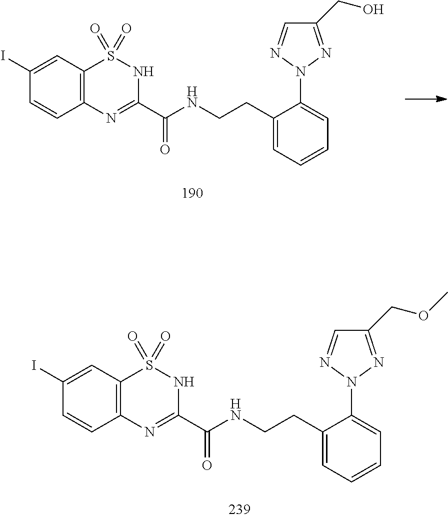 [SMILES: O=C(NCCc1ccccc1-n1ncc(CO)n1)C1=Nc2ccc(I)cc2S(=O)(=O)N1 ; COCc1cnn(-c2ccccc2CCNC(=O)C2=Nc3ccc(I)cc3S(=O)(=O)N2)n1]

To a suspension of N-(2-(4-(hydroxymethyl)-2H-1,2,3-triazol-2-yl)phenethyl)-7-iodo-2H-benzo[e][1,2,4]thiadiazine-3-carboxamide 1,1-dioxide 190 (0.050 g, 0.091 mmol) in acetonitrile (5 mL), under an atmosphere of nitrogen, was added silver(I)oxide (0.10 g, 0.45 mmol) and iodomethane (0.056 mL, 0.91 mmol). This was allowed to stir overnight at 50° C. The reaction was cooled and filtered through a pad of Celite®. The Celite® was washed with a mixture of DCM/MeOH and the filtrate was concentrated in vacuo. The solid residue was washed with warm DCM/MeOH (5 mL/1 mL) and the remaining solid was dissolved in DCM/MeOH (20 mL/10 mL), 1.25 M HCl in methanol (4 mL) was added and the solution sonicated for 5 minutes. The cloudy solution was filtered through a pad of Celite® and the filtrate was concentrated in vacuo to give the title compound (0.016 g, 31% yield) as a white solid. LCMS-B: rt 3.699 min; m/z 564.7 [M−H]⁻. ¹H NMR (400 MHz, DMSO-d₆) δ 12.68 (s, 1H), 9.29 (t, J=5.9 Hz, 1H), 8.12-8.06 (m, 2H), 8.05 (s, 1H), 7.61 (d, J=8.7 Hz, 1H), 7.57-7.37 (m, 4H), 4.58 (s, 2H), 3.46 (q, J=6.8 Hz, 2H), 2.93 (t, J=7.1 Hz, 2H). OCH₃ signal obscured by water. Presence confirmed via HMQC (3.33 ppm/57.9 ppm).

### Example 240: N-(2-(1H-pyrazol-1-yl)phenethyl)-7-iodo-2H-benzo[e][1,2,4]thiadiazine-3-carboxamide 1,1-dioxide 240

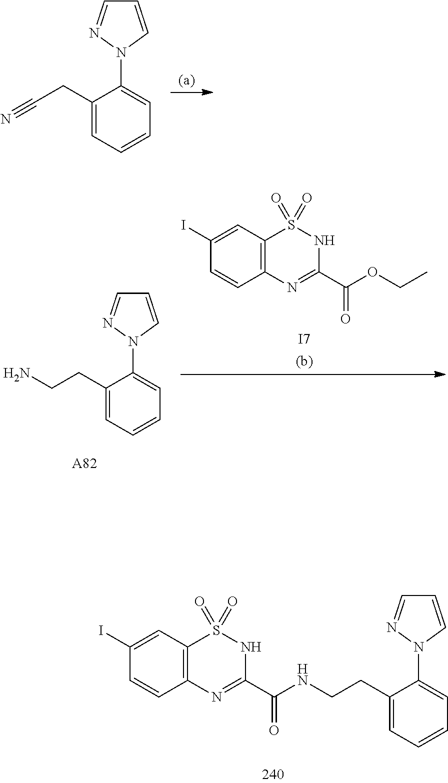

### a) 2-(2-(1H-pyrazol-1-yl)phenyl)ethan-1-amine A82

To 2-(2-pyrazol-1-ylphenyl)acetonitrile (0.13 g, 0.70 mmol) in THF (5 mL) was added borane-tetrahydrofuran complex 1.0 M solution in THF (3.5 mL, 3.5 mmol) dropwise. The solution was heated to reflux and allowed to stir overnight. The reaction was cooled and quenched slowly with water (5 mL). A 50% w/v aq. NaOH solution (2 mL) was added and the mixture was refluxed for 1 h. The reaction was cooled and the organics concentrated in vacuo. The remaining aqueous layer was extracted with DCM (10 mL×3), the organics were combined, washed with brine (20 mL), dried (Na₂SO₄) and concentrated in vacuo. The crude material was loaded onto a Biotage SCX cartridge (5 g) and washed with MeOH (30 mL), then a methanolic ammonia solution (30 mL). The methanolic washings were concentrated in vacuo to give the title compound (0.12 g, 90% yield) as a yellow oil. LCMS-B: rt 0.930 min; m/z 188.0 [M+H]⁺. ¹H NMR (400 MHz, Chloroform-d) δ 7.71 (dd, J=1.9, 0.7 Hz, 1H), 7.61 (dd, J=2.3, 0.7 Hz, 1H), 7.41-7.33 (m, 2H), 7.32-7.29 (m, 2H), 6.44 (t, J=2.1 Hz, 1H), 2.86-2.76 (m, 2H), 2.73-2.61 (m, 2H), 1.25 (br s, 2H).

### b) N-(2-(1H-pyrazol-1-yl)phenethyl)-7-iodo-2H-benzo[e][1,2,4]thiadiazine-3-carboxamide 1,1-dioxide 240

To a solution of 2-(2-(1H-pyrazol-1-yl)phenyl)ethan-1-amine A82 (0.049 g, 0.26 mmol) in EtOH (0.2 mL) was added ethyl 7-iodo-2H-benzo[e][1,2,4]thiadiazine-3-carboxylate 1,1-dioxide I7 (0.050 g, 0.13 mmol). The reaction was irradiated in a microwave reactor at 120° C. for 1 h, then cooled and the precipitate filtered. The solid was washed with EtOH (2 mL), then taken up in EtOAc (10 mL) and washed with 1M aqueous HCl (10 mL×2) and brine. A precipitate formed from the organic layer and this solid was collected by filtration to give the title compound (0.0080 g, 12% yield) a pale grey solid. LCMS-B: rt 3.354 min; m/z 521.6 [M+H]⁺. ¹H NMR (400 MHz, DMSO-d₆) δ 12.68 (br s, 1H), 9.41 (m, 1H), 8.11-8.03 (m, 2H), 8.00 (dd, J=2.3, 0.7 Hz, 1H), 7.72 (dd, J=1.8, 0.7 Hz, 1H), 7.59 (d, J=8.7 Hz, 1H), 7.48-7.31 (m, 4H), 6.48 (t, J=2.1 Hz, 1H), 3.45-3.36 (partially obscured by solvent, m, 2H), 2.83 (t, J=7.1 Hz, 2H).

### Example 241: N-(2-(1H-1,2,3-triazol-1-yl)phenethyl)-7-iodo-2H-benzo[e][1,2,4]thiadiazine-3-carboxamide 1,1-dioxide 241

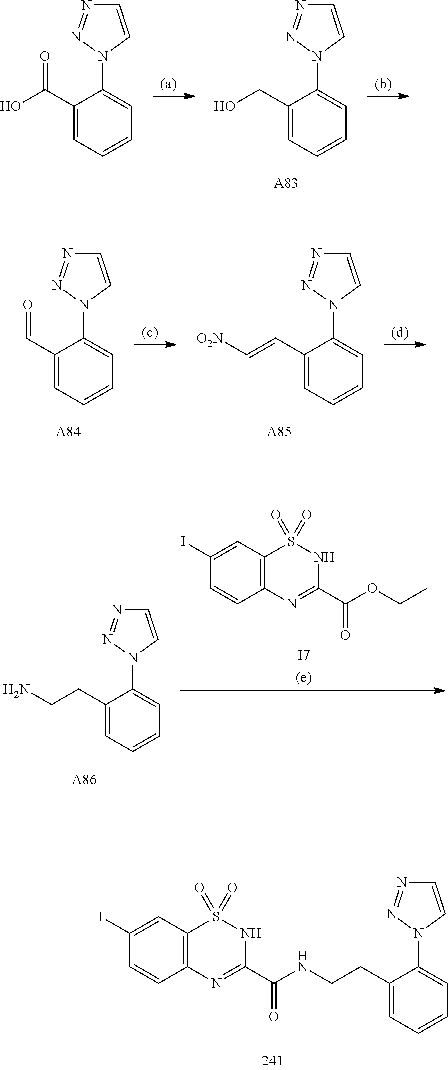

### a) (2-(1H-1,2,3-Triazol-1-yl)phenyl)methanol A83

A solution of 2-(triazol-1-yl)benzoic acid (0.50 g, 2.6 mmol) in tetrahydrofuran (10 mL) (note: required heat and sonication for complete dissolution), under an atmosphere of nitrogen, was cooled to 0° C. To this was added lithium aluminum hydride 1.0 M THF (3.96 mL, 3.96 mmol) dropwise over 15 min. After 10 min at this temperature, the reaction was allowed to warm to RT and stirred for a further 3 h. The reaction was cooled to 0° C. and cautiously added to 2M aqueous HCl (10 mL). The THF was removed in vacuo and the remaining aqueous phase extracted with DCM (10 mL×3). The combined organics were washed with brine (20 mL), dried (Na₂SO₄) and concentrated in vacuo to give the title compound (0.37 g, 80% yield) as an amber oil. LCMS-A: rt 4.364 min; m/z 176.0 [M+H]⁺. ¹H NMR (400 MHz, Chloroform-d) δ 7.96 (d, J=1.1 Hz, 1H), 7.89 (d, J=1.1 Hz, 1H), 7.64 (dd, J=7.2, 1.9 Hz, 1H), 7.56-7.45 (m, 2H), 7.39 (dd, J=7.7, 1.5 Hz, 1H), 4.48 (s, 2H), 3.44 (br s, 1H).

### b) 2-(1H-1,2,3-Triazol-1-yl)benzaldehyde A84

To a suspension of pyridinium chlorochromate (PCC) (0.91 g, 4.2 mmol) in DCM (6 mL), under an atmosphere of nitrogen, was added a solution of (2-(1H-1,2,3-triazol-1-yl)phenyl)methanol A83 (0.37 g, 2.1 mmol) in DCM (6 mL) dropwise. This was allowed to stir at RT for 1 h. Diethyl ether (10 mL) was added and the suspension filtered through a pad of Celite®. The pad was washed with diethyl ether (50 mL) and the filtrate concentrated in vacuo. The crude material was purified by column chromatography (Grace Biotage, 40 g SiO₂, 0-100% EtOAc in petroleum benzine 40-60° C.) to give the title compound (0.18 g, 49% yield) as a white solid. LCMS-A: rt 4.369 min; m/z 174.0 [M+H]⁺. ¹H NMR (400 MHz, Chloroform-d) δ 9.89 (d, J=0.7 Hz, 1H), 8.16-8.10 (m, 1H), 7.98 (d, J=1.1 Hz, 1H), 7.94 (d, J=1.2 Hz, 1H), 7.79 (td, J=7.7, 1.6 Hz, 1H), 7.72-7.64 (m, 1H), 7.53 (dd, J=7.8, 0.8 Hz, 1H).

### c) (E)-1-(2-(2-Nitrovinyl)phenyl)-1H-1,2,3-triazole A85

2-(1H-1,2,3-Triazol-1-yl)benzaldehyde A84 (0.16 g, 0.92 mmol), nitromethane (0.20 mL, 3.7 mmol) and ammonium acetate (0.036 g, 0.46 mmol) were added to glacial acetic acid (1 mL) and refluxed for 5 h. The reaction was cooled, poured into water (5 mL) and extracted with diethyl ether (3×5 mL). The organics were combined, washed with brine (10 mL), dried (Na₂SO₄) and concentrated in vacuo. The residue was recrystallised from EtOH to give the title compound (0.075 g, 38% yield) as a white solid. LCMS-A: rt 5.082 min; m/z 216.9 [M+H]⁺. ¹H NMR (400 MHz, Chloroform-d) δ 7.95 (d, J=1.1 Hz, 1H), 7.85 (d, J=1.1 Hz, 1H), 7.81 (d, J=13.6 Hz, 1H), 7.76 (ddd, J=7.6, 1.4, 0.8 Hz, 1H), 7.71-7.58 (m, 2H), 7.57-7.51 (m, 1H), 7.44 (d, J=13.6 Hz, 1H).

### d) 2-(2-(1H-1,2,3-Triazol-1-yl)phenyl)ethan-1-amine A86

To (E)-1-(2-(2-Nitrovinyl)phenyl)-1H-1,2,3-triazole A85 (0.072 g, 0.33 mmol) in dry THF (2 mL) at 0° C., under an atmosphere of nitrogen, was added lithium aluminum hydride 1.0 M THF (0.67 mL, 0.67 mmol) dropwise. This was allowed to warm to RT, then stirred for a further 3 h. The reaction was cooled to 0° C. and quenched with the slow addition of aqueous 1M NaOH (5 mL). Water (5 mL) and EtOAc (10 mL) were added and the layers separated. The aqueous was extracted with EtOAc (2×), the combined organics were washed with brine (20 mL), dried (Na₂SO₄) and concentrated in vacuo to give the title compound (0.043 g, 69% yield) as an oil. ¹H NMR (400 MHz, Chloroform-d) δ 7.93-7.61 (m, 2H), 7.52-7.29 (m, 6H), 2.81 (t, J=7.1 Hz, 2H), 2.58 (td, J=7.1, 2.4 Hz, 2H).

### e) N-(2-(1H-1,2,3-triazol-1-yl)phenethyl)-7-iodo-2H-benzo[e][1,2,4]thiadiazine-3-carboxamide 1,1-dioxide 241

To 2-(2-(1H-1,2,3-triazol-1-yl)phenyl)ethan-1-amine A86 (0.043 g, 0.23 mmol) in EtOH (0.125 mL) was added ethyl 7-iodo-2H-benzo[e][1,2,4]thiadiazine-3-carboxylate 1,1-dioxide I7 (0.056 g, 0.15 mmol). The reaction was irradiated in a CEM microwave at 120° C. for 2 h, then concentrated to dryness and partitioned between 1M aqueous HCl (2 mL) and EtOAc (2 mL). The layers were separated and the organic layer concentrated in vacuo. The material was taken up in minimum EtOH and Et₂O was added dropwise until a precipitate formed. The precipitate was collected and the process repeated. This material was further purified by column chromatography (Grace Biotage, 4 g SiO₂, 0-100% EtOAc in petroleum benzine 40-60° C., then 0-40% EtOAc in MeOH) to give the title compound (0.0050 g, 4.2% yield) as an off-white solid. ¹H NMR (400 MHz, DMSO-d₆) δ 12.68 (br s, 1H), 9.22 (br s, 1H), 8.43 (d, J=1.1 Hz, 1H), 8.10-8.01 (m, 2H), 7.94 (d, J=1.0 Hz, 1H), 7.65-7.36 (m, 5H), 2.72 (t, J=7.3 Hz, 2H). Two aliphatic protons obscured by the water signal.

### Example 242: N-((1-(oxazol-2-yl)cyclopentyl)methyl)-2H-benzo[e][1,2,4]thiadiazine-3-carboxamide 1,1-dioxide 242

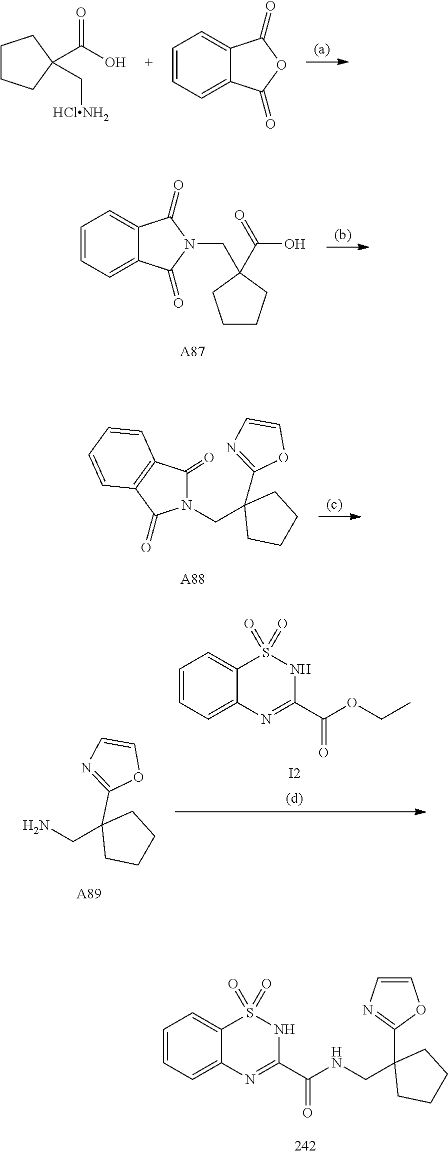

### a) 1-((1,3-Dioxoisoindolin-2-yl)methyl)cyclopentane-1-carboxylic Acid A87

To 1-(aminomethyl)cyclopentane-1-carboxylic acid hydrochloride (0.500 g, 2.783 mmol) in 1,4-dioxane (8 mL) was added NEt₃ (1.164 mL, 8.350 mmol). This was allowed to stir for 10 min, upon which phthalic anhydride (0.495 g, 3.340 mmol) was added. The mixture was sealed and irradiated in a microwave reactor at 150° C. for 30 min. The precipitated salts were filtered and the filtrate concentrated in vacuo. The material was taken up in minimal MeOH and loaded onto a 10 g Agilent, Bond Elut NH₂ column. The column was washed with 3 volumes of MeOH (3×30 mL), then stripped with 1M HCl in 1,4-dioxane (100 mL). The HCl wash was concentrated in vacuo to give the title compound (0.560 g, 74% yield) as a white solid. LCMS-B: rt 3.168 min; m/z 272.1 [M−H]⁻. ¹H NMR (400 MHz, DMSO-d₆): δ 12.34 (br s, 1H), 8.04-7.71 (m, 4H), 3.79 (s, 2H), 2.08-1.81 (m, 2H), 1.65-1.56 (m, 4H), 1.55-1.46 (m, 2H).

### b) 2-((1-(Oxazol-2-yl)cyclopentyl)methyl)isoindoline-1,3-dione A88

1-((1,3-Dioxoisoindolin-2-yl)methyl)cyclopentane-1-carboxylic acid A87 (0.300 g, 1.098 mmol) was dissolved in thionyl chloride (2 mL) and heated at 80° C. for 3 h. The remaining thionyl chloride was evaporated in vacuo. The residue was dissolved in sulfolane (2 mL), and to this was added 1,2,3-triazole (0.089 mL, 1.537 mmol) and K₂CO₃(0.303 g, 2.196 mmol). The reaction was heated to 150° C. for 30 min, then cooled, added to water (5 mL) and extracted with EtOAc (3×3 mL). The combined organics were washed with brine, dried (Na₂SO₄) and concentrated in vacuo. The crude material was purified by silica gel chromatography (Isolera Biotage 40 g SiO₂, 0-100% EtOAc in petroleum benzine 40-60° C.) to give the title compound (0.135 g, 42% yield) as a white solid. LCMS-B: rt 3.285 min, m/z 297.1 [M+H]⁺.

### c) (1-(Oxazol-2-yl)cyclopentyl)methanamine A89

To a suspension of 2-((1-(oxazol-2-yl)cyclopentyl)methyl)isoindoline-1,3-dione A88 (0.135 g, 0.456 mmol) in EtOH (6 mL) was added hydrazine hydrate (0.057 mL, 1.822 mmol). The solution was heated at 80° C. for 3 h, an additional portion of hydrazine hydrate (0.057 mL) was added, and the reaction was allowed to stir for a further 2 h. The reaction was cooled and the precipitate filtered and washed with a portion of cold EtOH (5 mL). The combined EtOH fractions were allowed to stand at 0° C. overnight, the precipitate was removed by filtration and the filtrate was loaded directly onto a 5 g SCX cartridge (Agilent Bond Elut) and the cartridge was washed with MeOH (20 mL), the product was then eluted with a 10% aq. NH₃ in MeOH solution (20 mL). The NH₃ washings were evaporated in vacuo give the title compound (0.049 g, 65% yield) as an oil. LCMS-B: rt 1.534 min, m/z 167.1 [M+H]⁺.

### d) N-((1-(Oxazol-2-yl)cyclopentyl)methyl)-2H-benzo[e][1,2,4]thiadiazine-3-carboxamide 1,1-dioxide 242

To (1-(oxazol-2-yl)cyclopentyl)methanamine A89 (0.045 g, 0.270 mmol) in EtOH (0.250 mL) was added ethyl 2H-benzo[e][1,2,4]thiadiazine-3-carboxylate 1,1-dioxide 12 (0.049 g, 0.193 mmol). The mixture was subjected to microwave irradiation at 100° C. for 30 min. The reaction was cooled and EtOH removed in vacuo. The residue was taken up in EtOAc (3 mL) and washed with 1M aqueous HCl (3 mL), brine (3 mL), dried (Na₂SO₄) and concentrated in vacuo to give the title compound (0.060 g, 84% yield) as a white solid. LCMS-B: rt 3.162 min; m/z 375.1 [M+H]⁺.

### Example 243: methyl (1-cyclohexyl-2-(1,1-dioxido-2H-benzo[e][1,2,4]thiadiazine-3-carboxamido)ethyl)carbamate 243

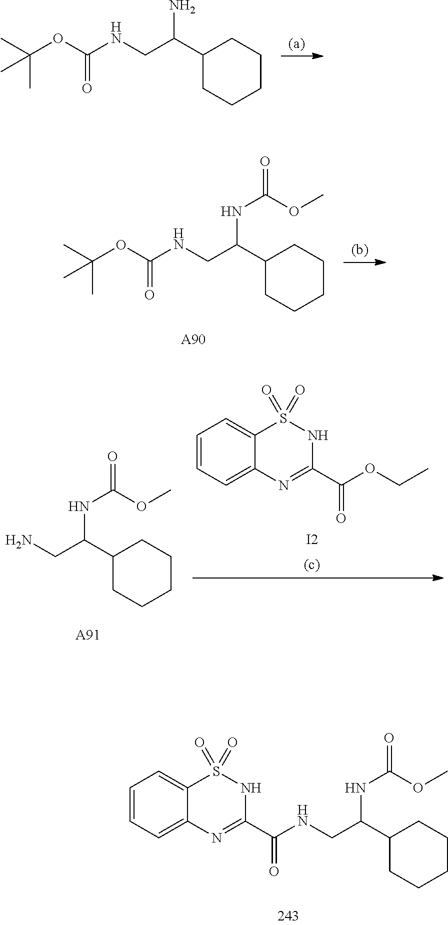

### a) tert-Butyl methyl (1-cyclohexylethane-1,2-diyl)dicarbamate A90

To a solution of the tert-butyl (2-amino-2-cyclohexylethyl)carbamate (0.500 g, 2.063 mmol) in DCM (15 mL) was added NEt₃ (0.316 mL, 2.269 mmol). This was allowed to stir for 10 min upon which the reaction was cooled to 0° C. and methyl chloroformate (0.189 mL, 2.269 mmol) was added dropwise. The reaction slowly warmed to RT and was allowed to stir overnight. 1M aqueous HCl (15 mL) was added and the layers separated. The organics were washed with saturated aqueous Na₂CO₃ (15 mL), brine (15 mL), dried (Na₂SO₄) and concentrated in vacuo to give the title compound (0.450 g, 73% yield) as a white solid. ¹H NMR (400 MHz, Chloroform-d): δ 4.86-4.69 (m, 1H), 3.65 (s, 3H), 3.58-3.45 (m, 1H), 3.20 (m, 2H), 1.81-1.62 (m, 6H), 1.42 (s, 9H), 1.29-0.95 (m, 4H).

### b) Methyl (2-amino-1-cyclohexylethyl)carbamate A91

To a solution of tert-butyl methyl (1-cyclohexylethane-1,2-diyl)dicarbamate A90 (0.450 g, 1.498 mmol) in DCM (6 mL) was added TFA (0.6 mL). This was allowed to stir at RT for 2 h upon which time the reaction was concentrated in vacuo to give the crude product. A portion of the crude material (0.162 g) in MeOH (˜1 mL) was gravity loaded onto a SCX cartridge (5 g). The cartridge was washed with 3 column volumes of MeOH, then 3 column volumes of a 10% solution of NH₃ in MeOH. The methanolic ammonia washes were combined and concentrated in vacuo to give the title compound (0.059 g) as a clear oil which was used directly in the next step.

### c) Methyl (1-cyclohexyl-2-(1,1-dioxido-2H-benzo[e][1,2,4]thiadiazine-3-carboxamido)ethyl)carbamate 243

To methyl (2-amino-1-cyclohexylethyl)carbamate A91 (0.059 g, 0.295 mmol) in EtOH (0.125 mL) was added ethyl 2H-benzo[e][1,2,4]thiadiazine-3-carboxylate 1,1-dioxide 12 (0.050 g, 0.197 mmol). The mixture was subjected to microwave irradiation at 100° C. for 30 min. The reaction was cooled and the solvent evaporated. The material was partitioned between 1M aqueous HCl (3 mL) and EtOAc (3 mL). The layers were separated and the organic phase was washed with brine (3 mL), dried (Na₂SO₄) and concentrated in vacuo to give the title compound (0.062 g, 77% yield) as a white solid. LCMS-A: rt 6.007 min; m/z 407.2 [M−H]⁻. ¹H NMR (400 MHz, DMSO-d₆) δ 12.65 (s, 1H), 9.03 (t, J=5.8, 5.8 Hz, 1H), 7.86 (dd, J=8.0, 1.4 Hz, 1H), 7.84-7.80 (m, 1H), 7.73 (ddd, J=8.5, 7.2, 1.5 Hz, 1H), 7.53 (ddd, J=8.2, 7.3, 1.2 Hz, 1H), 6.96 (d, J=9.1 Hz, 1H), 3.63-3.54 (m, 1H), 3.50 (s, 3H), 3.44 (dt, J=13.0, 5.4, 5.4 Hz, 1H), 3.24 (dt, J=13.7, 7.1, 7.1 Hz, 1H), 1.75-1.63 (m, 4H), 1.59 (d, J=10.0 Hz, 1H), 1.46-1.33 (m, 1H), 1.26-1.06 (m, 3H), 1.06-0.89 (m, 2H).

### Example 244: N-(2-(1H-pyrazol-1-yl)-2-(pyridin-2-yl)ethyl)-7-bromo-4H-benzo[e][1,2,4]thiadiazine-3-carboxamide 1,1-dioxide 244

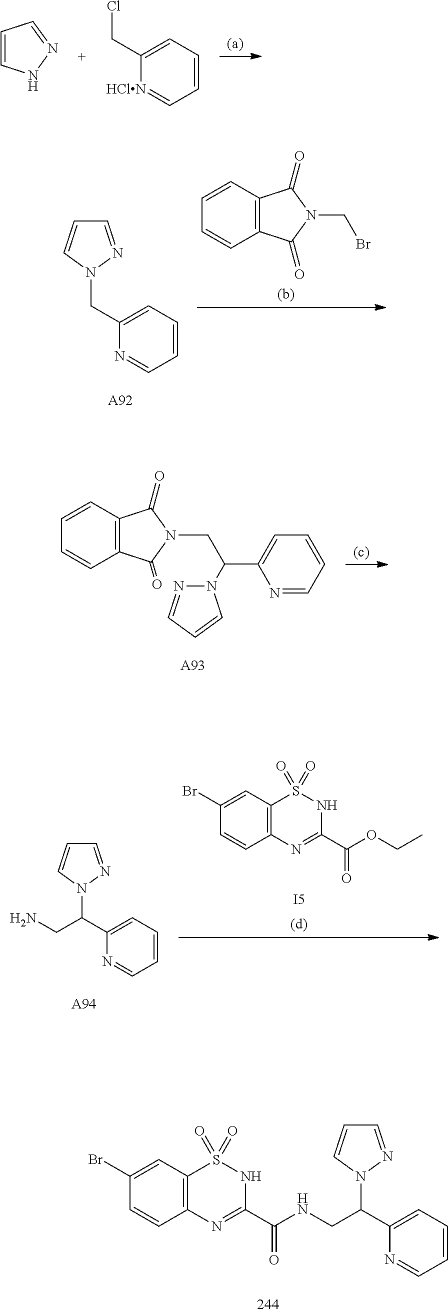

### a) 2-((1H-Pyrazol-1-yl)methyl)pyridine A92

To a solution of pyrazole (0.5 g, 7.35 mmol) in toluene (15 mL) was added 2-(chloromethyl)pyridine hydrochloride (1.44 g, 8.8 mmol), aqueous NaOH (40% w/v, 10 mL) and 40% w/v aqueous tetrabutylammonium hydrogen sulphate (catalytic 12 drops). The reaction mixture was heated at reflux for 20 hours and then partitioned between water (50 mL) and diethyl ether (3×50 mL). The combined organic layers were dried (MgSO₄) and evaporated in vacuo, and the crude product was purified by chromatography (24 g SiO₂ cartridge, 0-95% EtOAc in petroleum benzine 40-60° C.) to give the title compound (1.24 g, 89% yield) as a colourless viscous oil. ¹H NMR (400 MHz, Chloroform-d) δ 8.57 (d, J=6.14 Hz, 2H), 7.60 (d, J=1.80 Hz, 1H), 7.45 (d, J=2.33 Hz, 1H), 7.03 (d, J=6.15 Hz, 2H), 6.35 (t, J=2.12 Hz, 1H), 5.36 (s, 2H). LCMS-B: Rₜ 0.587 min, m/z 160.1 [M+H]⁺.

### b) 2-(2-(1H-Pyrazol-1-yl)-2-(pyridin-2-yl)ethyl)isoindoline-1,3-dione A93

To a solution of 2-((1H-pyrazol-1-yl)methyl)pyridine A92 (0.412 g, 2.59 mmol) in anhydrous THF (10 mL) at −78° C. under nitrogen was added N-(bromomethyl)phthalimide (0.808 g, 3.36 mmol) dropwise. A solution of lithium bis(trimethylsilyl)amide, 1.0 M solution in hexane (3.36 mL, 3.36 mmol) in anhydrous THF (8 mL) was then added dropwise and the mixture allowed to warm slowly to room temperature and stirred overnight. The mixture was diluted with a saturated aqueous NH₄Cl solution (50 mL) and water (25 mL), then extracted with DCM (50 mL×3). The combined organic extracts were washed with brine, dried over anhydrous MgSO₄, concentrated and purified by column chromatography (0-100% EtOAc in petroleum benzine 40-60° C.) to give the title compound (0.145 g, 18% yield) as a pale yellow solid. ¹H NMR (400 MHz, Chloroform-d) δ 8.61 (s, 2H), 7.81 (dd, J=3.07, 5.47 Hz, 2H), 7.72 (dd, J=3.06, 5.50 Hz, 2H), 7.59 (d, J=1.80 Hz, 1H), 7.51 (dd, J=0.60, 2.43 Hz, 1H), 7.43 (d, J=5.30 Hz, 2H), 6.27 (d, J=2.04 Hz, 1H), 5.99 (dd, J=6.38, 9.09 Hz, 1H), 4.63 (dd, J=9.14, 14.04 Hz, 1H), 4.41 (dd, J=6.40, 14.04 Hz, 1H). LCMS-A: Rₜ 4.60 min, m/z 318.9 [M+H]⁺.

### c) 2-(1H-Pyrazol-1-yl)-2-(pyridin-2-yl)ethan-1-amine A94

To a suspension of 2-(2-(1H-pyrazol-1-yl)-2-(pyridin-2-yl)ethyl)isoindoline-1,3-dione A93 (0.15 g, 0.46 mmol) in ethanol (30 mL) was added 64-65% v/v hydrazine hydrate (0.500 mL, 6.58 mmol) and the resulting solution was stirred at room temperature overnight. The mixture was filtered and the solid was washed with ethanol. The filtrate was partitioned between DCM (50 mL) and saturated aqueous NaHCO₃(50 mL). The layers were separated and the aqueous layer was extracted with DCM (100 mL×3). The combined organic extracts were washed with brine, dried over magnesium sulphate and concentrated to give the title compound (0.0550 g, 64% yield) as a yellow oil. ¹H NMR (400 MHz, Chloroform-d) δ 8.55 (s, 2H), 7.63 (s, 1H), 7.48 (d, J=2.46 Hz, 1H), 7.11-7.02 (m, 2H), 6.34 (s, 1H), 5.36-5.28 (m, 1H), 3.70 (obscured by solvent), 3.44-3.22 (m, 1H).

### d) N-(2-(1H-Pyrazol-1-yl)-2-(pyridin-2-yl)ethyl)-7-bromo-4H-benzo[e][1,2,4]thiadiazine-3-carboxamide 1,1-dioxide 244

Ethyl 7-bromo-2H-benzo[e][1,2,4]thiadiazine-3-carboxylate 1,1-dioxide 15 (65 mg, 0.20 mmol), 2-(1H-pyrazol-1-yl)-2-(pyridin-2-yl)ethan-1-amine A94 (0.055 g, 0.29 mmol) and absolute ethanol (0.5 mL) were heated in the microwave at 100° C. for 30 minutes. The reaction mixture was heated in the microwave once more at 100° C. for 30 minutes, then cooled to room temperature and filtered. The filtrate was dried in vacuo then purified by chromatography (4 g SiO₂ cartridge, 0-100% EtOAc in petroleum benzine 40-60° C. followed by 0-10% MeOH in EtOAc) to give the title compound as an off-white solid (2.7 mg, 2% yield). ¹H NMR (400 MHz, methanol-d₄) δ 8.50 (d, J=6.3 Hz, 2H), 7.99 (d, J=2.2 Hz, 1H), 7.85-7.80 (m, 2H), 7.64 (d, J=1.8 Hz, 1H), 7.53 (d, J=8.9 Hz, 1H), 7.32 (dd, J=4.8, 1.5 Hz, 2H), 6.38 (t, J=2.2 Hz, 1H), 5.87 (dd, J=8.6, 5.4 Hz, 1H), 4.31 (dd, J=13.9, 8.7 Hz, 1H), 4.14 (dd, J=13.9, 5.4 Hz, 1H). LCMS Rₜ 2.99 min, m/z 476.7 [M+H]⁺.

Assays

Acetyltransferase Biochemical Assay

Compounds may be tested for in vitro activity in the following assay:

To determine the inhibition of HAT enzymatic activity by test compounds, assay reactions were conducted in a volume of 8 μL in 384-well low volume assay plates. The reactions were performed in assay buffer (100 mM Tris-HCl, pH 7.8, 15 mM NaCl, 1 mM EDTA, 0.01% Tween-20, 1 mM Dithiothreitol, and 0.02% m/v chicken egg white albumin). Reactions were set up with 0.4 μM Acetyl coenzyme A (for all assays apart from KAT6A which was set up with 10 μM Acetyl coenzyme A), 100 nM of full-length recombinant histone labelled by limited biotinylation (KAT6A, KAT6B, KAT7: H3.1, KAT5, KAT8: H4), 10/5/8/40/20 nM of KAT5/KAT6A/KAT6B/KAT7/KAT8 enzyme respectively, and an acetyl-lysine specific antibody (H3.1: Cell Signaling Technology, H4: Abcam). 11-point dilution series of the test compounds were prepared in DMSO; a volume of 100 nL was transferred using a pin tool into assay plates containing substrates, before adding enzyme to start the reaction. Positive (no compound) and negative (AcCoA omitted) control reactions were included on the same plates and received the same amount of DMSO as the compound treated wells. After adding all reagents, the plates were sealed with adhesive seals and incubated for 90 min at room temperature. An additional 4 μL of assay buffer containing AlphaScreen® Protein A acceptor beads and Streptavidin donor beads (PerkinElmer, Waltham, Mass.) to a final concentration of 8 μg/mL was then added. After incubation for 2 hours the plates were read using an EnVision 2103 multi label plate reader (PerkinElmer) in HTS AlphaScreen® mode. IC₅₀ values were obtained from the raw readings by calculating percent inhibition (% I) for each reaction relative to controls on the same plate (% I=(I−CN)/(CP−CN) where CN/CP are the averages of the negative/positive reactions, respectively), then fitting the % l data vs. compound concentration [I] to % I=(A+((B−A)/(1+((C/[I])\hat{ }D)))) where A is the lower asymptote, B is the upper asymptote, C is the 1050 value, and D is the slope.

The results are shown in tables 1 to 5 below:

TABLE 1
(TIP60-KAT5)

| Example | IC50 (μM) |
|---|---|
| 1 | 0.286 |
| 2 | >125 |
| 3 | 96.5 |
| 4 | 5.33 |
| 5 | 1.17 |
| 6 | 90.1 |
| 7 | 11.7 |
| 8 | 4.4 |
| 9 | 12.4 |
| 10 | 79.7 |
| 11 | 11.8 |
| 12 | 2.62 |
| 13 | 0.727 |
| 14 | 2.36 |
| 15 | 1.4 |
| 16 | 23.8 |
| 17 | 51 |
| 18 | >125 |
| 19 | 33.2 |
| 20 | 29.5 |
| 22 | 72.4 |
| 23 | 3.73 |
| 24 | 7.16 |
| 25 | 6.89 |
| 26 | 1.66 |
| 27 | 1.29 |
| 28 | 63.5 |
| 29 | >125 |
| 30 | 112 |
| 31 | 15.1 |
| 32 | 5.04 |
| 33 | 6.95 |
| 34 | >125 |
| 35 | >125 |
| 36 | 19.6 |
| 37 | 44 |
| 38 | 1.96 |
| 39 | 5.88 |
| 40 | >125 |
| 41 | 0.0613 |
| 42 | 0.642 |
| 43 | 2.39 |
| 44 | >125 |
| 45 | 109 |
| 46 | 9.71 |
| 49 | >125 |
| 51 | 25.3 |
| 52 | 125 |
| 53 | 19.8 |
| 54 | 57.9 |
| 55 | 36.5 |
| 56 | 0.269 |
| 57 | 2.8 |
| 58 | 2.58 |
| 60 | 0.327 |
| 61 | >125 |
| 62 | >125 |
| 63 | >125 |
| 64 | 92.1 |
| 65 | 85.4 |
| 66 | >125 |
| 67 | >125 |
| 68 | 14.5 |
| 69 | >125 |
| 70 | >125 |
| 73 | 10.1 |
| 74 | >125 |
| 75 | 56.6 |
| 76 | >125 |
| 77 | 87 |
| 78 | 16.7 |
| 79 | 87.8 |
| 80 | >125 |
| 81 | 4.9 |
| 82 | 7.82 |
| 83 | 7.38 |
| 84 | 0.778 |
| 85 | 5.97 |
| 86 | 1.17 |
| 87 | 4.47 |
| 88 | 1.19 |
| 89 | 2.06 |
| 90 | 0.96 |
| 91 | 0.209 |
| 92 | 0.367 |
| 93 | 9.2 |
| 94 | 2.82 |
| 95 | 5.18 |
| 96 | 94.7 |
| 97 | >125 |
| 98 | >125 |
| 99 | >125 |
| 100 | 40.5 |
| 101 | >125 |
| 102 | 27.1 |
| 103 | >125 |
| 104 | >125 |
| 105 | 46.3 |
| 106 | >125 |
| 107 | 20.8 |
| 108 | 77.7 |
| 109 | 3.42 |
| 110 | 75.6 |
| 111 | 16.6 |
| 112 | 18.5 |
| 113 | >125 |
| 114 | 0.954 |
| 115 | 0.423 |
| 116 | 4.44 |
| 118 | 2.29 |
| 119 | 5.26 |
| 120 | 1.24 |
| 121 | 40.8 |
| 122 | >125 |
| 123 | 5.01 |
| 124 | 24.6 |
| 125 | >125 |
| 126 | 31.3 |
| 127 | 61.2 |
| 128 | >125 |
| 129 | >125 |
| 131 | >125 |
| 133 | >125 |
| 134 | 2.17 |
| 135 | 3.13 |
| 136 | 27.6 |
| 137 | 3.08 |
| 138 | 0.0952 |
| 139 | 1.3 |
| 142 | 4.27 |
| 143 | 13.8 |
| 144 | 1.65 |
| 145 | 18.9 |
| 146 | 0.0468 |
| 147 | 0.445 |
| 148 | 45.7 |
| 149 | 4.88 |
| 150 | 3.17 |
| 152 | 52.8 |
| 153 | 38.7 |
| 154 | >125 |
| 206 | 36.8 |

TABLE 2
(MOZ-KAT6A)

| Example | IC50 (μM) |
|---|---|
| 1 | 0.0241 |
| 2 | 38.1 |
| 3 | 7.66 |
| 4 | 0.1 |
| 5 | 0.667 |
| 7 | 2.8 |
| 9 | 0.0421 |
| 10 | 0.0906 |
| 11 | 1.81 |
| 13 | 0.229 |
| 15 | 0.211 |
| 16 | 1.37 |
| 17 | 3.33 |
| 18 | 3.12 |
| 19 | 1.35 |
| 20 | 6.05 |
| 22 | 1.98 |
| 23 | 0.056 |
| 24 | 0.127 |
| 25 | 0.0512 |
| 26 | 0.0287 |
| 27 | 0.0195 |
| 28 | 4.42 |
| 29 | 28.6 |
| 31 | 8.23 |
| 32 | 0.0498 |
| 33 | 0.126 |
| 34 | 51.6 |
| 35 | 59.9 |
| 36 | 0.661 |
| 37 | 0.771 |
| 38 | 0.532 |
| 41 | 0.0179 |
| 43 | 0.243 |
| 44 | 125 |
| 45 | 35.2 |
| 46 | 0.324 |
| 49 | 7.67 |
| 51 | 4.82 |
| 52 | 36.1 |
| 53 | 0.273 |
| 54 | 8.87 |
| 55 | 5.66 |
| 56 | 0.0809 |
| 61 | >125 |
| 62 | >125 |
| 63 | >125 |
| 64 | 102 |
| 65 | 53.9 |
| 66 | >125 |
| 67 | >125 |
| 68 | 3.81 |
| 69 | >125 |
| 70 | >125 |
| 74 | 67.5 |
| 75 | 1.72 |
| 76 | >125 |
| 77 | 35.5 |
| 78 | 3.74 |
| 79 | 35.4 |
| 80 | 58.8 |
| 81 | 1.61 |
| 82 | 9.19 |
| 83 | 2.61 |
| 84 | 0.256 |
| 85 | 8.1 |
| 86 | 2.7 |
| 87 | 8.93 |
| 91 | 0.594 |
| 92 | 0.783 |
| 93 | 2.2 |
| 96 | 1.17 |
| 97 | 4 |
| 98 | 36.1 |
| 99 | 15.8 |
| 100 | 41.7 |
| 101 | 10.2 |
| 102 | 10.6 |
| 103 | 125 |
| 107 | 12 |
| 108 | 11.6 |
| 112 | 4.58 |
| 113 | 125 |
| 114 | 0.861 |
| 115 | 0.476 |
| 116 | 2.26 |
| 131 | 62.3 |
| 133 | >125 |
| 134 | 0.149 |
| 135 | 0.168 |
| 136 | 8.03 |
| 137 | 0.107 |
| 139 | 0.464 |
| 142 | 0.0211 |
| 143 | 0.346 |
| 144 | 0.12 |
| 145 | 3.73 |
| 146 | 0.0259 |
| 147 | 0.645 |
| 148 | 5.39 |
| 149 | 0.102 |
| 152 | 47.5 |
| 154 | >125 |
| 206 | 9.64 |

TABLE 3
(HBO-KAT7)

| Example | IC50 (μM) |
|---|---|
| 1 | 0.0638 |
| 4 | 2.56 |
| 5 | 2.04 |
| 7 | 11.9 |
| 8 | 1.2 |
| 9 | 20.1 |
| 11 | 2.53 |
| 12 | 5.87 |
| 13 | 0.981 |
| 14 | 1.78 |
| 24 | 0.141 |
| 25 | 1.93 |
| 26 | 1.48 |
| 28 | 15.7 |
| 29 | 84.4 |
| 30 | 125 |
| 31 | 5.84 |
| 32 | 4.64 |
| 33 | 6.78 |
| 34 | 60.3 |
| 35 | 31.6 |
| 36 | 0.538 |
| 38 | 0.154 |
| 39 | 0.192 |
| 40 | 9.45 |
| 41 | 0.0944 |
| 42 | 0.255 |
| 43 | 1.99 |
| 46 | 2.65 |
| 54 | 4.01 |
| 55 | 4.51 |
| 56 | 0.219 |
| 57 | 2.53 |
| 58 | 1.59 |
| 60 | 0.555 |
| 68 | 0.462 |
| 73 | 26.4 |
| 78 | 4.56 |
| 79 | 29.5 |
| 80 | 104 |
| 84 | 0.0836 |
| 86 | 1.33 |
| 87 | 12 |
| 88 | 0.659 |
| 89 | 3.37 |
| 90 | 0.915 |
| 91 | 0.339 |
| 92 | 0.675 |
| 93 | 9.59 |
| 94 | 3.8 |
| 95 | 4.22 |
| 96 | 3.17 |
| 97 | 49.4 |
| 98 | 68.6 |
| 99 | 9.16 |
| 100 | 112 |
| 101 | >125 |
| 102 | 60.2 |
| 103 | >125 |
| 104 | >125 |
| 105 | >125 |
| 106 | >125 |
| 107 | 23.3 |
| 108 | 50.1 |
| 109 | 5.95 |
| 110 | 101 |
| 111 | 81.4 |
| 112 | 4.11 |
| 114 | 0.529 |
| 115 | 0.229 |
| 118 | 3.02 |
| 119 | 18.5 |
| 120 | 3.52 |
| 121 | 47 |
| 122 | >125 |
| 123 | 1.72 |
| 124 | 18.7 |
| 125 | >125 |
| 126 | 12.4 |
| 127 | 34.7 |
| 128 | >125 |
| 129 | >125 |
| 134 | 3.32 |
| 135 | 2.53 |
| 136 | 0.633 |
| 137 | 0.913 |
| 138 | 0.234 |
| 139 | 0.0615 |
| 142 | 6.57 |
| 143 | 1.75 |
| 146 | 0.16 |
| 147 | 0.167 |
| 153 | 3.41 |
| 173 | 0.051 |
| 174 | 7.49 |
| 175 | 0.162 |
| 176 | 0.207 |
| 177 | 0.064 |
| 178 | 0.571 |
| 206 | 7.17 |
| 216 | 0.063 |
| 217 | 1.74 |
| 233 | 0.038 |

TABLE 4
(MOF-KAT8)

| Example | IC50 (μM) |
|---|---|
| 1 | 14.6 |
| 4 | 28.8 |
| 5 | 27.7 |
| 7 | >125 |
| 24 | 69.6 |
| 25 | 78.2 |
| 26 | 23.7 |
| 32 | 74.1 |
| 33 | 89.6 |
| 41 | 4.87 |
| 46 | 88.2 |
| 82 | >125 |
| 84 | 33.3 |
| 86 | 39.4 |
| 87 | 114 |
| 88 | 47 |
| 91 | 12.1 |
| 92 | 21.8 |
| 114 | >125 |
| 115 | 31.2 |
| 116 | 56.7 |
| 136 | >125 |
| 137 | 100 |
| 138 | 8.07 |
| 139 | 19.1 |
| 142 | 56.7 |
| 143 | 30 |
| 146 | 4.19 |
| 147 | 26.1 |
| 162 | 4.20 |
| 163 | 9.78 |
| 164 | 29.6 |
| 165 | 43.1 |
| 167 | 6.48 |
| 168 | 3.39 |
| 169 | 5.14 |
| 170 | 3.75 |
| 171 | 41.7 |
| 172 | 5.13 |
| 173 | 39.1 |
| 174 | >125 |
| 175 | 29.3 |
| 176 | 92.9 |
| 177 | 6.25 |
| 178 | 106 |
| 179 | 10.4 |
| 180 | 77.0 |
| 181 | 104 |
| 182 | 50.0 |
| 183 | 36.3 |
| 184 | 9.22 |
| 186 | 71.5 |
| 187 | 22.8 |
| 188 | 39.8 |
| 189 | 7.96 |
| 190 | 48.9 |
| 203 | 103 |
| 204 | >125 |
| 208 | 24.1 |
| 212 | 40.8 |
| 213 | 3.54 |
| 214 | 7.058 |
| 215 | 8.74 |
| 216 | 64.3 |
| 217 | 22.5 |
| 218 | >125 |
| 219 | >125 |
| 228 | 6.52 |
| 233 | 6.13 |
| 234 | 57.3 |
| 235 | 6.59 |
| 236 | 20.2 |
| 237 | 6.35 |
| 238 | 41.4 |
| 239 | >125 |
| 243 | 82.5 |
| 244 | 82.9 |

TABLE 5
(QKF-KAT6B)

| Example | IC50 (μM) |
|---|---|
| 18 | 0.268 |
| 46 | 0.122 |

Histone H3 Lysine 14 Acetylation Biomarker Assay

Compounds may be tested for their ability to inhibit acetylation of the histone H3K14 marker (which is HBO1 mediated) in the following assay:

The cell line U2OS was seeded at a density of 12,000 cells per well in 96 well optical quality tissue culture plates in RPMI medium and 10% foetal bovine serum, and allowed to adhere for 24 hours under standard culture conditions (37 degree Celsius, 5% CO₂). At the end of this period the cells were washed with serum free medium. Compound dilutions prepared in DMSO were added to the serum free medium, with negative control wells reserved for treatment with DMSO only and positive controls receiving a potent inhibitor compound (e.g. Example 36 in WO2016/198507) at 10 μM concentration. After incubation for 24 hours, the cells were fixed with 3.7% formaldehyde in PBS for 20 minutes at room temperature, washed with phosphate buffer saline containing 0.1% Tween 20 and blocked with Odyssey blocking buffer (LI-COR, Lincoln, Nebr.) containing 0.1% TritonX100. Anti-H3K14ac specific antibody (Cell Signalling Technologies) in Odyssey blocking buffer containing 0.1% Tween 20 was added and incubated for 14 hours at 4 degree Celsius. After washing, a secondary antibody labelled with Alexa647 dye (LifeTechnologies) and Hoechst 33342 (1 μg/mL, SigmaAldrich) were added for 1 hour incubation. Plates were washed and read on a PerkinElmer Phenix high content imaging platform. Using a Columbus image analysis pipeline, individual nuclei were located by Hoechst 33342 stain and the acetylation level was calculated from the Alexa647-related intensity in the same area. The resulting mean intensity per cell was directly converted to percent inhibition relative to controls on the same plate and the data fitted against a four-parameter logistic model to determine the 50% inhibitory concentration (IC₅₀).

The results are shown in table 6 below:

| Example | IC50 (μM) |
|---|---|
| 1 | 0.317 |
| 4 | 30 |
| 8 | 9.68 |
| 36 | 9.98 |
| 38 | 1.5 |
| 39 | 2.49 |
| 41 | 0.0861 |
| 46 | 8.16 |
| 56 | 0.65 |
| 60 | 1.61 |
| 84 | 0.765 |
| 91 | 0.615 |
| 92 | 1.39 |
| 93 | 30 |
| 101 | 30 |
| 115 | 1.06 |
| 136 | 7.89 |
| 137 | 2.45 |
| 138 | 0.145 |
| 139 | 0.263 |
| 142 | 17.5 |
| 143 | 14.6 |
| 146 | 0.429 |
| 147 | 0.193 |

H₂A.Z Lysine 7 Acetylation Biomarker Assay

To discover a global TIP60/KAT5 cellular biomarker useful for monitoring PD responses of TIP60 inhibition in vitro and in vivo, various histone modifications were assessed for TIP60 dependence through genetic (TIP60 siRNA and CRISPR/Cas9) or TIP60 pharmacological inhibition. This analysis clearly identified acetylation of the histone variant H₂A.Z at Lysine 7 (H₂A.ZK7ac) as a global histone mark which is TIP60-dependent in both human and mouse cells. To a lesser extent, TIP60 also acetylated lysine 4 and 11 of H₂A.Z.

Compounds may be tested for their ability to inhibit the histone H₂A.Z Lysine 7 acetylation biomarker (which is TIP60 mediated) in the following assay:

The cell line U2OS was seeded at a density of 9,000 cells per well in 96 well optical quality tissue culture plates in RPMI medium and 10% foetal bovine serum, and allowed to adhere for 24 hours under standard culture conditions (37 degree Celsius, 5% CO₂). At the end of this period the cells were washed with serum free medium. Compound dilutions prepared in DMSO were added to the serum free medium, with negative control wells reserved for treatment with DMSO only and positive controls receiving a potent inhibitor compound (e.g. Example 146) at 20 μM concentration. After incubation for 24 hours, the cells were fixed with 3.7% formaldehyde in PBS for 20 minutes at room temperature, washed with phosphate buffer saline containing 0.1% Tween 20 and blocked with Odyssey blocking buffer (LI-COR, Lincoln, Nebr.) containing 0.1% TritonX100. Anti-H₂A.Z K7ac specific antibody (Abcam) in Odyssey blocking buffer containing 0.1% Tween 20 was added and incubated for 14 hours at 4 degree Celsius. After washing, a secondary antibody labelled with Alexa647 dye (LifeTechnologies) and Hoechst 33342 (10 μM, SigmaAldrich) were added for 1 hour incubation. Plates were washed and read on a PerkinElmer Phenix high content imaging platform. Using a Columbus image analysis pipeline, individual nuclei were located by Hoechst 33342 stain and the acetylation level was calculated from the Alexa647-related intensity in the same area. The resulting mean intensity per cell was directly converted to percent inhibition relative to controls on the same plate and the data fitted against a four-parameter logistic model to determine the 50% inhibitory concentration (IC₅₀).

The results are shown in table 7 below:

| Example | IC50 (μM) |
|---|---|
| 1 | 2.18 |
| 4 | 10 |
| 12 | 26.8 |
| 13 | 5.78 |
| 41 | 1 |
| 46 | 30 |
| 60 | 2.06 |
| 91 | 10 |
| 101 | 30 |
| 122 | 10 |
| 137 | 10 |
| 138 | 1.46 |
| 139 | 5.05 |
| 146 | 0.447 |
| 147 | 1.43 |

Further Assays

Protein Preparation

KAT5

Molecular Biology: A codon optimized DNA sequence (for expression in Escherichia cob) encoding amino acid residues 2 to 461 (Uniprot Q92993-2) of human KAT5 isoform was synthesised by GenScript USA Inc (Piscataway, N.J., USA). This was ligated into a modified pET43a E. coli expression vector designed to encode an N-terminal hexahistidine tag followed by a tobacco etch virus protease (TEV) cleavage site and by the KAT5 sequence. The resulting protein sequence is listed below.

MGHHHHHHGTENLYFQGSAEVGEIIEGCRLPVLRRNQDNEDEWPLAEILS
VKDISGRKLFYVHYIDFNKRLDEWVTHERLDLKKIQFPKKEAKTPTKNGL
PGSRPGSPEREVKRKVEVVSPATPVPSETAPASVFPQNGAARRAVAAQPG
RKRKSNCLGTDEDSQDSSDGIPSAPRMTGSLVSDRSHDDIVTRMKNIECI
ELGRHRLKPWYFSPYPQELTTLPVLYLCEFCLKYGRSLKCLQRHLTKCDL
RHPPGNEIYRKGTISFFEIDGRKNKSYSQNLCLLAKCFLDHKTLYYDTDP
FLFYVMTEYDCKGFHIVGYFSKEKESTEDYNVACILTLPPYQRRGYGKLL
IEFSYELSKVEGKTGTPEKPLSDLGLLSYRSYWSQTILEILMGLKSESGE
RPQITINEISEITSIKKEDVISTLQYLNLINYYKGQYILTLSEDIVDGHE
RAMLKRLLRIDSKCLHFTPKDWSKRGKWAS*

Protein Expression: To produce recombinant KAT5 protein, expression plasmid was transformed into E. coli BL21 DE3 strain and grown with shaking at 37° C. in 1 L volumes of Terrific broth (TB) supplemented with 100 μg/mL Ampicillin and 50 μM zinc until an OD600 of 0.8 was reached. Cultures were transferred to 18° C. and protein expression induced by the addition of Isopropyl β-D-1-thiogalactopyranoside to a final concentration of 0.5 mM and the cultures shaken overnight for further 16 hours. Following expression, cell cultures were centrifuged at 5000×g for 20 min and cell pellet stored frozen at −20° C.

Protein Purification: Protein purification was initiated by thawing the cell pellet (25 g wet weight) in Lysis buffer (50 mM Hepes pH 7.4, 500 mM NaCl, 5 mM imidazole, 5% [v/v] glycerol, 0.1% [w/v] CHAPS, 2 mM 2-mercaptoethanol, 3 mM MgCl₂, 0.5 mg/mL lysozyme, benzonase endonuclease [EMD Millipore], 1 mM PMSF, complete protease inhibitor tablets EDTA-free [Roche]) using a ratio of 6 mL of buffer per 1 g of cells. Cells were further lysed by sonication using a Misonix Liquid Processor (6×30 second pulses, amplitude 60 [70 watts]) and then centrifuged at 48,000×g at 4° C. Supernatant (cell lysate) was mixed with 20 mL of Q-Sepharose FF resin (GE Healthcare) pre-equilibrated with Q buffer (20 mM Hepes pH 7.4, 1 M NaCl). The unbound fraction from Q-Sepharose FF was then incubated with 5 mL of cOmplete His-Tag Purification Resin (Roche), pre-equilibrated with IMAC Wash Buffer (20 mM hepes pH 7.4, 500 mM NaCl, 35 mM imidazole). The resin was washed with IMAC Wash Buffer, and bound KAT5 eluted with IMAC Elution buffer (20 mM hepes pH 7.4, 500 mM NaCl, 300 mM imidazole). IMAC-eluted protein was immediately desalted into Storage buffer (50 mM Na citrate pH 6.5, 500 mM NaCl, 5% [v/v] glycerol) using 2× HiPrep 26/10 desalting columns (GE Healthcare) in series. Desalted protein was further purified by passing through a HiLoad 26/60 Superdex 75 column pre-equilibrated in Storage buffer. Finally, KAT5 protein was concentrated to 1.5 mg/mL using Amicon Ultra centrifugal filter unit (Utra-15 MWCO 10 kDa), flash-frozen in liquid nitrogen and stored in −70° C. freezer.

KAT6A

Molecular Biology: The DNA sequence encoding amino acid residues 507 to 778 (Uniprot Q92794-1) of human KAT6A was amplified by PCR and was ligated into a modified pET E. coli expression vector designed to encode a NusA solubility tag followed by a hexahistidine tag and a tobacco etch virus protease (TEV) cleavage site and by the KAT6A sequence. The resulting protein sequence is listed below.

MNKEILAVVEAVSNEKALPREKIFEALESALATATKKKYEQEIDVRVQID
RKSGDFDTFRRWLVVDEVTQPTKEITLEAARYEDESLNLGDYVEDQIESV
TFDRITTQTAKQVIVQKVREAERAMVVDQFREHEGEIITGVVKKVNRDNI
SLDLGNNAEAVILREDMLPRENFRPGDRVRGVLYSVRPEARGAQLFVTRS
KPEMLIELFRIEVPEIGEEVIEIKAAARDPGSRAKIAVKTNDKRIDPVGA
CVGMRGARVQAVSTELGGERIDIVLWDDNPAQFVINAMAPADVASIVVDE
DKHTMDIAVEAGNLAQAIGRNGQNVRLASQLSGWELNVMTVDDLQAKHQA
EAHAAIDTFTKYLDIDEDFATVLVEEGFSTLEELAYVPMKELLEIEGLDE
PTVEALRERAKNALATIAQAQEESLGDNKPADDLLNLEGVDRDLAFKLAA
RGVCTLEDLAEQGIDDLADIEGLTDEKAGALIMAARNICWFGDEATSGSG
HHHHHHSAGENLYFQGAMGRCPSVIEFGKYEIHTWYSSPYPQEYSRLPKL
YLCEFCLKYMKSRTILQQHMKKCGWFHPPVNEIYRKNNISVFEVDGNVST
IYCQNLCLLAKLFLDHKTLYYDVEPFLFYVLTQNDVKGCHLVGYFSKEKH
CQQKYNVSCIMILPQYQRKGYGRFLIDFSYLLSKREGQAGSPEKPLSDLG
RLSYMAYWKSVILECLYHQNDKQISIKKLSKLTGICPQDITSTLHHLRML
DFRSDQFVIIRREKLIQDHMAKLQLNLRPVDVDPECLRWTP*

Protein Expression: To produce recombinant KAT6A protein, expression plasmid was transformed into E. coli BL21 DE3 strain and grown with shaking at 37° C. in 1 L volumes of Terrific broth (TB) supplemented with 100 μg/mL Ampicillin until an OD600 of 0.8 was reached. Cultures were transferred to 18° C. and protein expression induced by the addition of Isopropyl β-D-1-thiogalactopyranoside to a final concentration of 0.5 mM and the cultures shaken overnight for further 16 hours. Following expression, cell cultures were centrifuged at 5000×g for 20 min and cell pellet stored frozen at −20° C.

Protein Purification: Protein purification was initiated by thawing the cell pellet (40 g wet weight) in Lysis buffer (25 mM Tris-HCl pH 7.8, 500 mM NaCl, 5 mM DTT, 0.01% [v/v] Triton-X 100, 5% [v/v] glycerol, 2 mM MgCl₂, 10 mM Imidazole, 0.5 mg/mL lysozyme, benzonase endonuclease [EMD Millipore], 1 mM PMSF, complete protease inhibitor tablets EDTA-free [Roche]) using a ratio of 5 mL of buffer per 1 g of cells. Cells were further lysed by 3 passes (at 15000 psi) through an ice cooled Avestin C₅ cell crusher and then centrifuged at 48,000×g at 4° C. Supernatant (cell lysate) was filtered through a 5 μm filter and applied onto 5 mL HiTrap IMAC Sepharose FF column (GE Healthcare) pre-equilibrated with IMAC wash buffer (25 mM Tris-HCl pH 7.8, 500 mM NaCl, 5 mM DTT, 0.01% [v/v] Triton-X 100, 5% [v/v] glycerol, 20 mM Imidazole) using a Profinia Affinity chromatography purification system (Bio-Rad). The IMAC column was then washed with IMAC Wash buffer and bound KAT6A protein eluted with IMAC Elution buffer (25 mM Tris-HCl pH 7.8, 500 mM NaCl, 5% [v/v] glycerol, 5 mM DTT, 250 mM Imidazole). IMAC-eluted protein was further purified by passing through a HiLoad 26/60 Superdex 200 column pre-equilibrated in Storage buffer (25 mM Tris-HCl pH 7.8, 500 mM NaCl, 5 mM DTT, 5% [v/v] glycerol). Finally, KAT6A protein was concentrated to 1 mg/mL using Amicon Ultra centrifugal filter unit (Utra-15 MWCO 10 kDa), flash-frozen in liquid nitrogen and stored in −70° C. freezer.

KAT6B was obtained from SignalChem, catalog ID: K315-381BG

KAT7

Molecular Biology: A codon optimized DNA sequence encoding amino acid residues 325 to 611 (Uniprot 095251-1) of human KAT7 was synthesised by GenScript USA Inc (Piscataway, N.J., USA). This was ligated into a modified pET43a E. coli expression vector designed to encode an N-terminal hexahistidine tag followed by a tobacco etch virus protease (TEV) cleavage site and by the KAT7 sequence. The resulting protein sequence is listed below.

MGHHHHHHGTENLYFQGSRLQGQITEGSNMIKTIAFGRYELDTWYHSPYP
EEYARLGRLYMCEFCLKYMKSQTILRRHMAKCVWKHPPGDEIYRKGSISV
FEVDGKKNKIYCQNLCLLAKLFLDHKTLYYDVEPFLFYVMTEADNTGCHL
IGYFSKEKNSFLNYNVSCILTMPQYMRQGYGKMLIDFSYLLSKVEEKVGS
PERPLSDLGLISYRSYWKEVLLRYLHNFQGKEISIKEISQETAVNPVDIV
STLQALQMLKYWKGKHLVLKRQDLIDEWIAKEAKRSNSNKTMDPSCLKWT
PPKGTAS

Protein Expression: To produce recombinant KAT7 protein, expression plasmid was transformed into E. coli BL21 DE3 RIL strain and grown with shaking at 37° C. in 1 L volumes of Terrific broth (TB) supplemented with 100 μg/mL Ampicillin and 50 μM zinc until an OD600 of 0.8 was reached. Cultures were transferred to 18° C. and protein expression induced by the addition of Isopropyl β-D-1-thiogalactopyranoside to a final concentration of 0.5 mM and the cultures shaken overnight for further 16 hours. Following expression, cell cultures were centrifuged at 5000×g for 20 min and cell pellet stored frozen at −20° C.

Protein Purification: Protein purification was initiated by thawing the cell pellet (10 g wet weight) in Lysis buffer (50 mM Hepes pH 7.5, 300 mM NaCl, 5 mM DTT, 5 mM Imidazole, 0.05% [v/v] Brij 35, 10% [v/v] glycerol, 3 mM MgCl₂, 0.5 mg/mL lysozyme, benzonase endonuclease [EMD Millipore], 1 mM PMSF, complete protease inhibitor tablets EDTA-free [Roche]) using a ratio of 10 mL of buffer per 1 g of cells. Cells were further lysed by sonication using a Misonix Liquid Processor (6×30 second pulses, amplitude 60 [70 watts]) and then centrifuged at 48,000×g at 4° C. Supernatant (cell lysate) was incubated with 1 mL of cOmplete His-Tag Purification Resin (Roche), pre-equilibrated with IMAC Wash Buffer 1 (25 mM Hepes pH 7.5, 800 mM NaCl, 5 mM imidazole, 10% [v/v] glycerol, 5 mM DTT, 0.01% [v/v] Brij 35, 50 mM arginine, 50 mM glutamic acid). The resin was sequentially washed with IMAC Wash buffer 1 and IMAC Wash buffer 2 (25 mM hepes pH 7.5, 300 mM NaCl, 20 mM imidazole, 10% [v/v] glycerol, 5 mM DTT, 0.01% [v/v] Brij 35, 50 mM arginine, 50 mM glutamic acid). Bound KAT7 protein was eluted with IMAC Elution buffer (25 mM hepes pH 7.5, 200 mM NaCl, 500 mM imidazole, 10% [v/v] glycerol, 5 mM DTT 0.01% [v/v] Brij 35, 50 mM arginine, 50 mM glutamic acid). The eluting protein was collected directly into 4 volumes of Desalt Buffer (50 mM Na citrate pH 6.5, 200 mM NaCl, 0.01% [v/v] Brij 35, 10% [v/v] glycerol, 5 mM DTT) to bring the final imidazole concentration to 100 mM. IMAC-eluted protein was immediately desalted into Desalt buffer using 2× HiPrep 26/10 desalting columns (GE Healthcare) in series. Desalted protein was further purified by passing through a HiLoad 26/60 Superdex 75 column pre-equilibrated in Storage Buffer (50 mM Na citrate pH 6.5, 200 mM NaCl, 10% [v/v] glycerol, 5 mM DTT). Finally, KAT7 protein was concentrated to 3.5 mg/mL using Amicon Ultra centrifugal filter unit (Utra-15 MWCO 10 kDa), flash-frozen in liquid nitrogen and stored in −70° C. freezer.

KAT8

Molecular Biology: A codon optimized DNA sequence (for expression in E. coli) encoding amino acid residues 177 to 447 (Uniprot Q9H7Z6-1) of human KAT8 was synthesised by Thermo Fisher Scientific GENEART GmbH (Regensberg, Germany). This was ligated into pPROEX Hta E. coli expression vector designed to encode an N-terminal hexahistidine tag followed by a tobacco etch virus protease (TEV) cleavage site and by the KAT8 sequence. The resulting protein sequence is listed below.

MSYYHHHHHHDYDIPTTENLYFQGAKYVDKIHIGNYEIDAWYFSPFPEDY
GKQPKLWLCEYCLKYMKYEKSYRFHLGQCQWRQPPGKEIYRKSNISVYEV
DGKDHKIYCQNLCLLAKLFLDHKTLYFDVEPFVFYILTEVDRQGAHIVGY
FSKEKESPDGNNVACILTLPPYQRRGYGKFLIAFSYELSKLESTVGSPEK
PLSDLGKLSYRSYWSWVLLEILRDFRGTLSIKDLSQMTSITQNDIISTLQ
SLNMVKYWKGQHVICVTPKLVEEHLKSAQYKKPPITVDSVCLKWAP*

Protein Expression: To produce recombinant KAT8 protein, expression plasmid was transformed into E. coli BL21 DE3 strain and grown with shaking at 37° C. in 1 L volumes of

Terrific broth (TB) supplemented with 100 μg/mL Ampicillin until an OD600 of 0.8 was reached. Cultures were transferred to 18° C. and protein expression induced by the addition of Isopropyl β-D-1-thiogalactopyranoside to a final concentration of 0.5 mM and the cultures shaken overnight for further 16 hours. Following expression, cell cultures were centrifuged at 5000×g for 20 min and cell pellet stored frozen at −20° C.

Protein Purification: Protein purification was initiated by thawing the cell pellet (34 g wet weight) in Lysis buffer (20 mM Hepes pH 7.5, 500 mM NaCl, 5 mM Imidazole, 5% [v/v] glycerol, 0.01% [v/v] Triton-X 100, 5 mM 2-mercaptoethanol, 2 mM MgCl₂, 0.5 mg/mL lysozyme, benzonase endonuclease [EMD Millipore], 1 mM PMSF, complete protease inhibitor tablets EDTA-free [Roche]) using a ratio of 3 mL of buffer per 1 g of cells. Cells were further lysed by 3 passes (at 15000 psi) through an ice cooled Avestin C₅ cell crusher and then centrifuged at 48,000×g at 4° C. Supernatant (cell lysate) was filtered through a 0.2 μm filter and applied onto 5 mL HiTrap IMAC Sepharose FF column (GE Healthcare) pre-equilibrated with IMAC wash buffer 1 (20 mM Hepes pH 7.5, 500 mM NaCl, 0.5 mM TCEP, 5 mM Imidazole) using a Profinia Affinity chromatography purification system (Bio-Rad). The IMAC column was then sequentially washed with IMAC Wash buffer 1 and IMAC Wash buffer 2 (20 mM Hepes pH 7.5, 500 mM NaCl, 0.5 mM TCEP, 10 mM Imidazole) and bound KAT8 protein eluted with IMAC Elution buffer (20 mM Hepes pH 7.5, 500 mM NaCl, 0.5 mM TCEP, 500 mM Imidazole). IMAC-eluted protein was further purified by passing through a HiLoad 26/60 Superdex 200 column pre-equilibrated in Storage buffer (20 mM Hepes pH 7.5, 500 mM NaCl, 1 mM TCEP). Finally, KAT8 protein was concentrated to 0.2 mg/mL using Amicon Ultra centrifugal filter unit (Utra-15 MWCO 10 kDa), flash-frozen in liquid nitrogen and stored in −70° C. freezer.

Revised Acetyltransferase Biochemical Assay

To determine the inhibition of KAT enzymatic activity by test compounds, assay reactions were conducted in a volume of 8 μL in 384-well low volume assay plates. The reactions were performed in assay buffer (100 mM Tris-HCl, pH 7.8, 15 mM NaCl, 1 mM EDTA, 0.01% Tween-20, 1 mM Dithiothreitol, and 0.01% m/v chicken egg white albumin).

Reactions were set up with 1 μM Acetyl coenzyme A, 100 nM of full-length recombinant histone labelled by limited biotinylation (KAT6A, KAT6B, KAT7: H3.1, KAT5, KAT8: H4), 10/5/8/40/20 nM of KAT5/KAT6A/KAT6B/KAT7/KAT8 enzyme respectively, and an acetyl-lysine specific antibody (H3.1: Cell Signaling Technology, H4: Abcam). 11-point dilution series of the test compounds were prepared in DMSO; a volume of 100 nL was transferred using a pin tool into assay plates containing substrates, before adding enzyme to start the reaction. Positive (no compound, DMSO only) and negative (AcCoA omitted) control reactions were included on the same plates and received the same amount of DMSO as the compound treated wells. After adding all reagents, the plates were sealed with adhesive seals and incubated for 90 min at room temperature. An additional 4 μL of assay buffer containing AlphaScreen® Protein A acceptor beads and Streptavidin donor beads (PerkinElmer, Waltham, Mass.) to a final concentration of 8 μg/mL was then added. After incubation for 2 hours the plates were read using an EnVision 2103 multi label plate reader (PerkinElmer) in HTS AlphaScreen® mode. IC₅₀ values were obtained from the raw readings by calculating percent inhibition (% I) for each reaction relative to controls on the same plate (% I=(I−CN)/(CP−CN) where CN/CP are the averages of the negative/positive reactions, respectively), then fitting the %1 data vs. compound concentration [I] to % I=(A+((B−A)/(1+((C/[I])\hat{ }D)))) where A is the lower asymptote, B is the upper asymptote, C is the IC₅₀ value, and D is the slope.

The results are shown in tables 8 to 12 below:

TABLE 8
(MOZ-KAT6A)

| Example | IC50 (μM) |
|---|---|
| 1 | 0.005 |
| 2 | 5.139 |
| 3 | 4.954 |
| 5 | 0.069 |
| 6 | 18.658 |
| 7 | 0.316 |
| 8 | 0.011 |
| 13 | 0.010 |
| 14 | 0.010 |
| 15 | 0.682 |
| 19 | 0.270 |
| 20 | 0.490 |
| 23 | 0.120 |
| 24 | 0.110 |
| 25 | 0.064 |
| 26 | 0.041 |
| 32 | 0.030 |
| 33 | 0.047 |
| 34 | 5.782 |
| 36 | 0.074 |
| 39 | 0.032 |
| 41 | 0.005 |
| 43 | 0.014 |
| 46 | 0.064 |
| 49 | 1.685 |
| 50 | 6.186 |
| 56 | 0.010 |
| 57 | 0.403 |
| 59 | 0.032 |
| 60 | 0.010 |
| 68 | 0.108 |
| 75 | 0.308 |
| 78 | 0.203 |
| 81 | 0.552 |
| 84 | 0.017 |
| 86 | 0.096 |
| 91 | 0.024 |
| 93 | 0.098 |
| 96 | 0.149 |
| 97 | 0.417 |
| 113 | 98.977 |
| 118 | 0.046 |
| 120 | 0.017 |
| 129 | 0.250 |
| 134 | 0.024 |
| 135 | 0.047 |
| 139 | 0.100 |
| 144 | 0.021 |
| 145 | 0.649 |
| 146 | 0.002 |
| 147 | 0.029 |
| 150 | 1.022 |
| 153 | 0.054 |
| 155 | 0.595 |
| 157 | 8.797 |
| 158 | 1.732 |
| 159 | 0.371 |
| 160 | 0.471 |
| 161 | 0.269 |
| 162 | 0.029 |
| 163 | 0.017 |
| 164 | 0.017 |
| 165 | 0.031 |
| 166 | 0.007 |
| 167 | 0.004 |
| 168 | 0.008 |
| 169 | 0.023 |
| 170 | 0.143 |
| 171 | 0.024 |
| 172 | 0.005 |
| 173 | 0.011 |
| 174 | 0.573 |
| 175 | 0.013 |
| 176 | 0.076 |
| 177 | 0.004 |
| 178 | 0.021 |
| 179 | 0.005 |
| 180 | 0.229 |
| 181 | 0.032 |
| 182 | 0.006 |
| 183 | 0.044 |
| 184 | 0.008 |
| 185 | 0.042 |
| 186 | 0.024 |
| 187 | 0.015 |
| 188 | 0.041 |
| 189 | 0.075 |
| 190 | 0.008 |
| 191 | 0.043 |
| 192 | 0.613 |
| 193 | 0.493 |
| 194 | 5.564 |
| 195 | 0.209 |
| 196 | 0.080 |
| 197 | 0.290 |
| 198 | 0.351 |
| 199 | 0.838 |
| 200 | 9.800 |
| 201 | 0.268 |
| 202 | 1.043 |
| 203 | 0.427 |
| 204 | 0.122 |
| 205 | 0.970 |
| 206 | 1.391 |
| 208 | 1.597 |
| 209 | 0.378 |
| 210 | 0.303 |
| 211 | 2.180 |
| 212 | 0.241 |
| 213 | 0.002 |
| 214 | 0.009 |
| 215 | 0.004 |
| 216 | 0.028 |
| 217 | 0.265 |
| 218 | 0.153 |
| 219 | 3.586 |
| 220 | 0.020 |
| 221 | 0.572 |
| 222 | 0.131 |
| 223 | 0.216 |
| 224 | 0.165 |
| 225 | 0.447 |
| 226 | 0.075 |
| 227 | 1.362 |
| 228 | 0.007 |
| 230 | 0.761 |
| 231 | 0.100 |
| 232 | 0.252 |
| 233 | 0.013 |
| 234 | 0.072 |
| 235 | 0.009 |
| 236 | 0.010 |
| 237 | 0.010 |
| 238 | 0.188 |
| 239 | 0.017 |
| 240 | 0.021 |
| 241 | 0.082 |
| 242 | 2.774 |
| 243 | 12.281 |
| 244 | 6.828 |

TABLE 9
(HBO-KAT7)

| Example | IC50 (μM) |
|---|---|
| 1 | 0.076 |
| 2 | 28.029 |
| 3 | 49.934 |
| 6 | 21.294 |
| 7 | 1.176 |
| 8 | 0.134 |
| 13 | 0.128 |
| 14 | 0.083 |
| 15 | 0.874 |
| 19 | 1.003 |
| 20 | 1.253 |
| 23 | 2.884 |
| 24 | 0.583 |
| 25 | 12.045 |
| 26 | 5.071 |
| 32 | 0.356 |
| 33 | 0.551 |
| 34 | 11.469 |
| 36 | 3.380 |
| 39 | 0.299 |
| 41 | 0.059 |
| 43 | 0.086 |
| 46 | 1.078 |
| 49 | 3.133 |
| 50 | 49.069 |
| 56 | 0.063 |
| 57 | 0.840 |
| 59 | 0.403 |
| 60 | 0.201 |
| 68 | 0.601 |
| 75 | 1.148 |
| 78 | 3.526 |
| 81 | 4.600 |
| 84 | 0.062 |
| 86 | 0.787 |
| 91 | 0.074 |
| 93 | 1.794 |
| 96 | 1.114 |
| 113 | 6.411 |
| 118 | 0.412 |
| 120 | 0.140 |
| 129 | 24.812 |
| 134 | 0.720 |
| 135 | 0.419 |
| 139 | 0.184 |
| 144 | 1.387 |
| 146 | 0.036 |
| 147 | 0.057 |
| 150 | 3.594 |
| 153 | 0.672 |
| 155 | 9.516 |
| 157 | 22.305 |
| 158 | 5.465 |
| 159 | 0.295 |
| 160 | 1.662 |
| 161 | 4.387 |
| 162 | 0.512 |
| 163 | 0.115 |
| 164 | 0.242 |
| 165 | 1.768 |
| 166 | 0.183 |
| 167 | 0.062 |
| 168 | 0.621 |
| 169 | 0.386 |
| 170 | 1.635 |
| 171 | 0.785 |
| 172 | 0.041 |
| 173 | 0.161 |
| 174 | 4.150 |
| 175 | 0.478 |
| 176 | 0.869 |
| 177 | 0.124 |
| 178 | 0.112 |
| 179 | 0.028 |
| 180 | 0.557 |
| 181 | 0.320 |
| 182 | 0.237 |
| 183 | 0.718 |
| 184 | 0.114 |
| 185 | 0.264 |
| 186 | 1.962 |
| 187 | 0.115 |
| 188 | 0.215 |
| 189 | 0.214 |
| 190 | 0.414 |
| 191 | 0.243 |
| 192 | 2.304 |
| 193 | 1.937 |
| 194 | 26.048 |
| 195 | 2.215 |
| 196 | 0.025 |
| 197 | 7.530 |
| 198 | 8.374 |
| 199 | 6.566 |
| 200 | >125 |
| 201 | 56.499 |
| 202 | >125 |
| 203 | 1.671 |
| 204 | 59.533 |
| 205 | 2.728 |
| 206 | 1.207 |
| 208 | 4.509 |
| 209 | 1.675 |
| 210 | 1.121 |
| 211 | 6.072 |
| 212 | 1.091 |
| 213 | 0.111 |
| 214 | 0.050 |
| 215 | 0.020 |
| 216 | 0.152 |
| 217 | 1.189 |
| 218 | 9.410 |
| 219 | 104.980 |
| 220 | 0.214 |
| 221 | 0.072 |
| 222 | 0.023 |
| 223 | 1.008 |
| 224 | 0.204 |
| 225 | 1.460 |
| 226 | 1.926 |
| 227 | 4.485 |
| 228 | 0.092 |
| 230 | 2.300 |
| 231 | 0.143 |
| 232 | 0.393 |
| 233 | 0.014 |
| 234 | 0.089 |
| 235 | 0.115 |
| 236 | 0.073 |
| 237 | 0.121 |
| 238 | 0.881 |
| 239 | 0.686 |
| 240 | 0.134 |
| 241 | 0.948 |
| 242 | 32.984 |
| 243 | 77.338 |
| 244 | 3.835 |

TABLE 10
(TIP60-KAT5)

| Example | IC50 (μM) |
|---|---|
| 1 | 0.068 |
| 2 | 60.736 |
| 3 | 99.577 |
| 5 | 0.493 |
| 6 | >125 |
| 7 | 5.922 |
| 8 | 2.009 |
| 13 | 0.111 |
| 14 | 0.156 |
| 15 | 5.547 |
| 19 | 14.646 |
| 20 | 13.769 |
| 23 | 1.733 |
| 24 | 5.402 |
| 25 | 5.914 |
| 26 | 5.936 |
| 32 | 5.330 |
| 33 | 3.780 |
| 34 | 70.321 |
| 36 | 5.471 |
| 39 | 3.060 |
| 41 | 0.032 |
| 43 | 0.266 |
| 46 | 4.050 |
| 49 | >125 |
| 50 | >125 |
| 56 | 0.061 |
| 57 | 0.725 |
| 59 | 0.721 |
| 60 | 0.058 |
| 68 | 7.215 |
| 75 | 14.078 |
| 78 | 4.541 |
| 81 | 6.652 |
| 84 | 0.426 |
| 86 | 0.521 |
| 91 | 0.090 |
| 93 | 1.999 |
| 96 | 13.329 |
| 97 | 26.114 |
| 113 | >125 |
| 118 | 0.208 |
| 120 | 0.224 |
| 129 | 58.315 |
| 134 | 0.648 |
| 135 | 0.646 |
| 139 | 0.707 |
| 144 | 1.061 |
| 145 | 7.455 |
| 146 | 0.013 |
| 147 | 0.132 |
| 150 | 1.375 |
| 153 | 3.374 |
| 155 | 2.685 |
| 157 | >125 |
| 158 | 26.795 |
| 159 | 3.201 |
| 160 | 13.225 |
| 161 | 9.163 |
| 162 | 1.541 |
| 163 | 0.221 |
| 164 | 0.781 |
| 165 | 6.015 |
| 166 | 0.714 |
| 167 | 0.056 |
| 168 | 0.458 |
| 169 | 0.412 |
| 170 | 13.255 |
| 171 | 1.161 |
| 172 | 0.025 |
| 173 | 0.651 |
| 174 | 15.259 |
| 175 | 0.311 |
| 176 | 5.114 |
| 177 | 0.023 |
| 178 | 0.852 |
| 179 | 0.029 |
| 180 | 0.249 |
| 181 | 0.123 |
| 182 | 0.284 |
| 183 | 0.068 |
| 184 | 0.099 |
| 185 | 0.994 |
| 186 | 0.734 |
| 187 | 0.242 |
| 188 | 1.439 |
| 189 | 2.845 |
| 190 | 0.303 |
| 191 | 0.919 |
| 192 | 11.112 |
| 193 | 4.167 |
| 194 | 125.000 |
| 195 | 1.847 |
| 196 | 0.818 |
| 197 | 23.574 |
| 198 | 42.346 |
| 199 | 15.551 |
| 200 | >125 |
| 201 | 43.711 |
| 202 | >125 |
| 203 | 3.750 |
| 204 | >125 |
| 205 | 30.020 |
| 206 | 13.658 |
| 208 | 13.297 |
| 209 | 8.447 |
| 210 | 10.867 |
| 211 | 24.658 |
| 212 | 4.003 |
| 213 | 0.193 |
| 214 | 0.070 |
| 215 | 0.025 |
| 216 | 0.506 |
| 217 | 1.458 |
| 218 | 16.764 |
| 219 | >125 |
| 220 | 1.432 |
| 221 | 1.573 |
| 222 | 0.149 |
| 223 | 3.325 |
| 224 | 9.008 |
| 225 | 5.124 |
| 226 | 3.728 |
| 227 | 98.725 |
| 228 | 0.111 |
| 230 | 4.899 |
| 231 | 0.306 |
| 232 | 2.741 |
| 233 | 0.154 |
| 234 | 1.368 |
| 235 | 0.034 |
| 236 | 0.113 |
| 237 | 0.163 |
| 238 | 1.815 |
| 239 | 0.597 |
| 240 | 0.309 |
| 241 | 1.011 |
| 242 | 122.908 |
| 243 | 39.941 |
| 244 | 14.557 |

TABLE 11
(MOF-KAT8)

| Example | IC50 (μM) |
|---|---|
| 1 | 4.541 |
| 2 | >125 |
| 3 | 12.168 |
| 7 | 81.608 |
| 8 | 10.526 |
| 13 | 36.448 |
| 14 | 37.823 |
| 19 | >125 |
| 20 | 62.808 |
| 25 | >125 |
| 26 | 39.893 |
| 32 | >125 |
| 33 | >125 |
| 41 | 9.785 |
| 43 | 71.630 |
| 46 | 99.430 |
| 56 | 1.303 |
| 57 | 11.346 |
| 59 | 26.833 |
| 60 | 23.981 |
| 68 | 16.547 |
| 75 | >125 |
| 78 | >125 |
| 84 | 77.003 |
| 86 | 42.366 |
| 91 | 21.080 |
| 93 | >125 |
| 96 | >125 |
| 113 | >125 |
| 118 | >125 |
| 120 | 41.456 |
| 129 | >125 |
| 134 | 15.671 |
| 139 | 75.833 |
| 144 | 46.671 |
| 146 | 2.857 |
| 147 | 28.611 |
| 153 | 20.085 |
| 157 | >125 |
| 158 | 30.651 |
| 159 | 16.307 |
| 160 | 4.889 |
| 161 | 22.952 |
| 162 | 34.488 |
| 163 | 14.704 |
| 164 | 34.379 |
| 165 | >125 |
| 166 | 36.777 |
| 167 | 8.402 |
| 168 | 26.451 |
| 169 | 43.737 |
| 170 | >125 |
| 171 | >125 |
| 172 | 6.098 |
| 173 | 30.359 |
| 175 | 30.171 |
| 176 | 30.179 |
| 177 | 8.206 |
| 178 | 60.964 |
| 179 | 9.661 |
| 181 | 31.222 |
| 182 | 24.460 |
| 183 | 30.515 |
| 184 | 10.244 |
| 187 | 14.120 |
| 188 | 54.274 |
| 189 | 28.697 |
| 190 | 68.365 |
| 196 | 78.602 |
| 203 | 114.969 |
| 204 | >125 |
| 213 | 15.171 |
| 214 | 20.058 |
| 215 | 5.724 |
| 216 | 58.551 |
| 218 | >125 |
| 219 | >125 |
| 220 | 26.838 |
| 225 | >125 |
| 226 | >125 |
| 227 | >125 |
| 228 | 9.660 |
| 231 | 6.533 |
| 232 | 34.952 |
| 233 | 9.251 |
| 234 | 23.550 |
| 235 | 3.227 |
| 236 | 19.618 |
| 237 | 15.260 |
| 238 | 25.625 |
| 239 | 75.640 |
| 240 | 62.623 |

TABLE 12
(QKF-KAT6B)

| Example | IC50 (μM) |
|---|---|
| 1 | 0.060 |
| 8 | 0.210 |
| 14 | 0.058 |
| 25 | 0.610 |
| 26 | 0.120 |
| 32 | 0.155 |
| 36 | 0.724 |
| 41 | 0.028 |
| 46 | 0.589 |
| 60 | 0.039 |
| 91 | 0.350 |
| 93 | 1.782 |
| 113 | >125 |
| 144 | 0.459 |
| 146 | 0.019 |
| 147 | 0.311 |
| 159 | 4.049 |
| 163 | 0.117 |
| 166 | 0.072 |
| 167 | 0.027 |
| 168 | 0.037 |
| 172 | 0.281 |
| 179 | 0.088 |
| 181 | 0.077 |
| 182 | 0.059 |
| 196 | 0.991 |
| 197 | 0.780 |
| 198 | 1.383 |
| 199 | 6.172 |
| 201 | 5.259 |
| 202 | >125 |
| 203 | 3.313 |
| 204 | >125 |
| 213 | 0.022 |
| 215 | 0.065 |
| 220 | 0.134 |
| 221 | 4.335 |
| 231 | 3.239 |
| 233 | 0.254 |
| 238 | 5.869 |

Histone H3 Lysine 14 Acetylation Biomarker Assay

Compounds may be tested for their ability to inhibit acetylation of the histone H3 Lysine 14 (which is HBO1 mediated) marker in the following assay:

The cell line U2OS was seeded at a density of 3,000 cells per well in 384-well optical quality tissue culture plates in RPMI medium supplemented with 10% foetal bovine serum and 10 mM Hepes. The cells were allowed to adhere for 24 hours under standard culture conditions (37 degree Celsius, 5% CO2). At the end of this period the cells were washed with serum free medium. Compound dilutions prepared in DMSO were added to the serum free medium, with negative control wells reserved for treatment with DMSO only and 100% inhibition positive controls receiving a potent inhibitor compound (e.g. (Z)-4-fluoro-N-((3-hydroxyphenyl)sulfonyl)-5-methyl-[1,1′-biphenyl]-3-carbohydrazonic acid) at 10 μM concentration. After incubation for 24 hours, the cells were fixed with 4% formaldehyde in PBS for 15 minutes at room temperature, washed with phosphate buffer saline and blocked with blocking buffer containing 0.2% TritonX100 and 2% BSA. Anti-H3K14ac specific antibody (Cell Signalling Technologies) in blocking buffer was added and incubated overnight at 4 degree Celsius. After washing, a secondary antibody labelled with AlexaFluor 488 dye (ThermoFisher) and Hoechst 33342 (1 μg/mL, Life Technologies) were added for 2 hours incubation at room temperature. Plates were washed and read on a

PerkinElmer Opera HCS high content imaging platform. Using a Columbus image analysis pipeline, individual nuclei were located by Hoechst 33342 stain and the acetylation level was calculated from the AlexaFluor 488-related intensity in the same area. The resulting mean intensity per cell was converted to percent inhibition relative to controls on the same plate and the data fitted against a four-parameter logistic model to determine the 50% inhibitory concentration (IC50).

The results are shown in Table 13 below:

TABLE 13

| Example | IC50 (μM) |
|---|---|
| 162 | 6.52 |
| 163 | 0.892 |
| 164 | 2.08 |
| 166 | 0.611 |
| 167 | 0.349 |
| 168 | 6.44 |
| 169 | 1.30 |
| 171 | 10.4 |
| 172 | 2.87 |
| 175 | 1.84 |
| 176 | 4.43 |
| 177 | 1.03 |
| 179 | 0.219 |
| 181 | 35.3 |
| 182 | 0.488 |
| 186 | >40.0 |
| 187 | 0.491 |
| 188 | 0.427 |
| 189 | >20.0 |
| 190 | 4.95 |
| 193 | 31.2 |
| 196 | 0.095 |
| 201 | >40.0 |
| 203 | 2.26 |
| 212 | 4.15 |
| 213 | 4.94 |
| 214 | 3.60 |
| 215 | 0.221 |
| 216 | 14.1 |
| 217 | 1.29 |
| 220 | 0.917 |
| 221 | 1.66 |
| 222 | 0.437 |
| 225 | >40.0 |
| 226 | 24.4 |
| 228 | 3.25 |
| 231 | 3.88 |
| 233 | 3.20 |
| 237 | 0.498 |
| 238 | 13.7 |
| 239 | 19.2 |
| 240 | 3.32 |
| 244 | >40.0 |

H₂A.Z Lysine 7 Acetylation Biomarker Assay

Compounds may be tested for their ability to inhibit the histone H₂A.Z Lysine 7 acetylation marker (which is TIP60 mediated) in the following assay:

The cell line U2OS was seeded at a density of 3,000 cells per well in 384-well optical quality tissue culture plates in RPMI medium supplemented with 10% foetal bovine serum and 10 mM Hepes. The cells were allowed to adhere for 24 hours under standard culture conditions (37 degree Celsius, 5% CO₂). At the end of this period the cells were washed with serum free medium. Compound dilutions prepared in DMSO were added to the serum free medium, with negative control wells reserved for treatment with DMSO only and 100% inhibition positive controls receiving a potent inhibitor compound enantiomer 1 of 7-iodo-N-(2-(oxazol-2-yl)-2-phenylethyl)-2H-benzo[e][1,2,4]thiadiazine-3-carboxamide 1,1-dioxide, which is compound 146, at 30 μM concentration. After incubation for 24 hours, the cells were fixed with 4% formaldehyde in PBS for 15 minutes at room temperature, washed with phosphate buffer saline and blocked with blocking buffer containing 0.2% TritonX100 and 2% BSA. Anti-H₂A.ZK7ac specific antibody (Abcam) in blocking buffer was added and incubated overnight at 4 degree Celsius. After washing, a secondary antibody labelled with AlexaFluor 488 dye (ThermoFisher) and Hoechst 33342 (1 μg/mL, Life Technologies) were added for 2 hours incubation at room temperature. Plates were washed and read on a PerkinElmer Opera HCS high content imaging platform. Using a Columbus image analysis pipeline, individual nuclei were located by Hoechst 33342 stain and the acetylation level was calculated from the AlexaFluor 488-related intensity in the same area. The resulting mean intensity per cell was converted to percent inhibition relative to controls on the same plate and the data fitted against a four-parameter logistic model to determine the 50% inhibitory concentration (IC₅₀).

The results are shown in Table 14 below:

TABLE 14

| Example | IC50 (μM) |
|---|---|
| 162 | 10.9 |
| 163 | 1.52 |
| 164 | 2.82 |
| 166 | 5.35 |
| 167 | 0.516 |
| 168 | 8.85 |
| 169 | 9.88 |
| 171 | >40.0 |
| 172 | 2.09 |
| 175 | 11.9 |
| 176 | 15.4 |
| 177 | 1.18 |
| 178 | 20.5 |
| 179 | 1.10 |
| 180 | 37.5 |
| 181 | >40.0 |
| 182 | 8.10 |
| 183 | 33.2 |
| 184 | 8.28 |
| 186 | >40.0 |
| 187 | 5.46 |
| 189 | >40.0 |
| 190 | 31.1 |
| 191 | >40.0 |
| 193 | >40.0 |
| 196 | 0.882 |
| 201 | >40.0 |
| 203 | >40.0 |
| 204 | >40.0 |
| 213 | 29.5 |
| 214 | 8.78 |
| 215 | 4.44 |
| 216 | 15.9 |
| 217 | 32.1 |
| 220 | 7.81 |
| 221 | 6.87 |
| 222 | 1.28 |
| 225 | >40.0 |
| 226 | >40.0 |
| 228 | 5.71 |
| 231 | 4.52 |
| 233 | 1.99 |
| 234 | 6.15 |
| 235 | 1.08 |
| 236 | 7.85 |
| 237 | 3.41 |
| 240 | 11.21 |
| 241 | >40.0 |

Histone H3 Lysine 23 Acetylation Biomarker Assay

Compounds may be tested for their ability to inhibit acetylation of the histone H3K23 marker, which is KATE mediated, in the following assay:

The cell line U2OS was seeded at a density of 9,000 cells per well in 96 well optical quality tissue culture plates in RPMI medium and 10% foetal bovine serum, and allowed to adhere for 24 hours under standard culture conditions (37 degree Celsius, 5% CO₂). At the end of this period the medium was aspirated. Compound dilutions prepared in DMSO were added to medium, with negative control wells reserved for treatment with DMSO only and 100% inhibition positive controls receiving a potent inhibitor compound (e.g. cas 2055397-28-7, benzoic acid, 3-fluoro-5-(2-pyridinyl)-, 2-[(2-fluorophenyl)sulfonyl]hydrazide) (Baell, J., Nguyen, H. N., Leaver, D. J., Cleary, B. L., Lagiakos, H. R., Sheikh, B. N., Thomas. T. J., Aryl sulfonohydrazides, WO2016198507A1, 2016) at 10 μM concentration and 200 μL transferred to the cells. After incubation for 24 hours, the cells were fixed with 3.7% formaldehyde in PBS for 20 minutes at room temperature, washed (5×5 minutes) with phosphate buffer saline containing 0.1% Tween 20 and blocked with Odyssey blocking buffer (LI-COR, Lincoln, Nebr.) containing 0.1% TritonX100. Anti-H3K23ac specific antibody (Abcam ab177275) in Odyssey blocking buffer containing 0.1% Tween 20 was added and incubated for 16 hours at 4 degree Celsius. After washing (as above), a secondary antibody labelled with Alexa647 dye (LifeTechnologies) and Hoechst 33342 (1 μg/mL, SigmaAldrich) were added for 1 hour incubation. Plates were washed as previously and read on a PerkinElmer Phenix high content imaging platform. Using a Columbus image analysis pipeline, individual nuclei were located by Hoechst 33342 stain and the acetylation level was calculated from the Alexa647-related intensity in the same area. The resulting mean intensity per cell was directly converted to percent inhibition relative to controls on the same plate and the data fitted against a four-parameter logistic model to determine the 50% inhibitory concentration (IC50).

The results are shown in Table 15 below:

TABLE 15

| Example | IC50 (μM) |
|---|---|
| 1 | 0.064 |
| 8 | 5.865 |
| 14 | 1.063 |
| 25 | 3.822 |
| 26 | 1.078 |
| 32 | >10 |
| 36 | 0.263 |
| 41 | 0.035 |
| 46 | 0.178 |
| 57 | >10 |
| 60 | 1.418 |
| 91 | 7.687 |
| 93 | >10 |
| 97 | >10 |
| 113 | >10 |
| 144 | 0.104 |
| 146 | 0.016 |
| 147 | 0.482 |
| 159 | 5.089 |
| 163 | 0.453 |
| 166 | 0.093 |
| 167 | 0.057 |
| 168 | 0.525 |
| 172 | >10 |
| 175 | 0.154 |
| 177 | 0.195 |
| 179 | 0.112 |
| 181 | >10 |
| 182 | 0.084 |
| 186 | >10 |
| 193 | 9.078 |
| 196 | 1.009 |
| 197 | 3.040 |
| 198 | 5.198 |
| 199 | 10.000 |
| 201 | >10 |
| 202 | >10 |
| 203 | >10 |
| 204 | >10 |
| 213 | 0.116 |
| 215 | 0.953 |
| 220 | 0.540 |
| 221 | >10 |
| 222 | 7.148 |
| 231 | >10 |

### REFERENCES

Aggarwal and Calvi, Nature, 2004, 430, 372-376 doi:10.1038/nature02694
Avvakumov et al., Oncogene, 2007, 26, 5395-5407 doi:10.1038/sj.onc.1210608
Berge et al., J. Pharm. Sci., 1977, 66, 1-19 doi:10.1002/jps.2600660104
Borrow et al., Nat. Genet., 1996, 14, 33-41 doi:10.1038/ng0996-33
Dekker et al., Drug, Discov. Today, 2014, 19, 654-660 doi:10.1016/j.drudis.2013.11.012
Doyon et al., Mol. Cell., 2006, 21, 51-64 doi:10.1016/j.molcel.2005.12.007
Dhuban et al., Sci. Immunol., 2017, 2, 9297 doi:10.1126/sciimmunol.aai9297
Duong et al., Cancer Res., 2013, 73, 5556-5568 doi:10.1158/0008-5472.CAN-13-0013
Ghizzoni et al., Eur. J. Med. Chem., 2012, 47, 337-344 doi:10.1016/j.ejmech.2011.11.001
Gil et al., J. Proteomics, 2017, 150, 297-309 doi:10.1016/j.jprot.2016.10.003
Gobert, M. et al., Cancer Research, 2009, 69, 2000-2009 doi:10.1158/0008-5472.CAN-08-2360
Holbert et al., J. Biol. Chem., 2007, 282, 36603-36613 doi:10.1074/jbc.M705812200
Iizuka et al., Mol. Cell. Biol., 2006, 26, 1098-1108 doi:10.1128/MCB.26.3.1098-1108.2006
Iizuka et al., Cancer Sci., 2013, 104, 1647-1655 doi:10.1111/cas.12303
Jeong, et al., Blood Res 2016 51(3), 152-154 doi:10.5045/br.2016.51.3.152
Joshi, et al., Immunity 2015, 43, 579-590 doi:10.1016/j.immuni.2015.08.006
Li, B. et al., PNAS, 2007, 104, 4571-4576 doi:10.1073/pnas.0700298104
Melero, et al. Nature Reviews Cancer, 2015, 15, 457-472 doi:10.1038/nrc3973
Merson et al., J. Neurosci., 2006, 26, 11359-11370 doi:10.1523/JNEUROSCI.2247-06.2006
Miller, A. M. et al. J. Immunol., 2006, 177, 7398-7405 doi:10.4049/jimmunol.177.10.7398
Persa, E. et al. Cancer Letters, 2015 368(2), 252-261 doi:10.1016/j.canlet.2015.03.003
Sheikh et al., Blood, 2015, 125(12), 1910-21 doi:10.1182/blood-2014-08-594655
Shi et al, Nature Biotech, 2015, 33, 661-667 doi:10.1038/nbt.3235
Su et al., Int. J. Mol. Sci., 2016, 17, 1-18 doi:10.3390/ijms17101594
Stern et al., Crit. Rev. Oncol. Hematol., 2005, 54, 11-29 doi:10.1016/j.critrevonc.2004.10.011
Thomas et al., Development, 2000, 127, 2537-2548 PMID:10821753
Tao, H. et al., Lung Cancer, 2012, 75, 95-101 doi:10.1016/j.lungcan.2011.06.002
Turner-Ivey et al., Neoplasia, 2014, 16(8): 644-655 doi:10.1016/j.neo.2014.07.007
Valerio et al., Cancer Research, 2017, 77(7), 1753-62 doi:10.1158/0008-5472.CAN-16-2374
Vizmanos et al., Genes Chromosomes Cancer, 2003, 36(4), 402-405 doi:10.1002/gcc.10174
Voss et al., BioEssays, 2009, 31(10), 1050-1061 doi:10.1002/bies.200900051
Wang, L., et al. EBioMedicine, 2016, 13, 99-156 doi:10.1016/j.ebiom.2016.10.018
Wang, X. et al., Oncogene, 2017, 36, 3048-3058 doi:10.1038/onc.2016.458
Xiao, Y. et al., Cell reports, 2014, 7, 1471-1480 doi:10.1016/j.celrep.2014.04.021
Yan, M. et al., Breast Cancer Research, 2011, 13, R47 doi:10.1186/bcr2869
Zack et al., Nature Genetics 2013 45, 1134-1140 doi:10.1038/ng.2760
Zhang et al., Mini. Rev. Med. Chem., 2017, 17, 1-8 doi:10.2174/1389557516666160923125031

1. A compound of formula I: 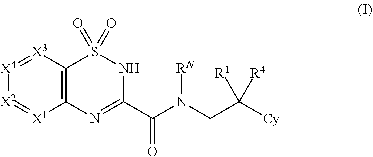
wherein:
R^{N} is H or Me;
X⁴ is selected from CY and N;
X¹, X² and X³ are each selected from CH and N, where none or one of X¹, X², X³ and X⁴ are N;
Y is selected from the group consisting of: H; halo; cyano; R², where R² is selected from CH₃, CH₂F, CHF₂ and CF₃; ethynyl; cyclopropyl; OR³, where R³ is selected from H, CH₃, CH₂F, CHF₂ and CF₃; NR^{N1}R^{N2}, where R^{N1} and R^{N2} are independently selected from H and CH₃; COQ¹, where Q¹ is selected from C₁₋₄ alkyl, OH, OC₁₋₄ alkyl and NR^{N1}R^{N2}; NHSO₂Q³, where Q³ is C₁₋₃ alkyl; pyridyl; C₅ heteroaryl, which may be substituted by a group selected from C₁₋₃ alkyl, which itself may be substituted by OH or CONR^{N1}R^{N2}; SO₂Me; C₁₋₃ alkyl, substituted by NHZ, where Z is H, Me, SO₂Me, or COMe; and C₁₋₃ alkyl, substituted by OH;
Cy is selected from pyridyl, oxazolyl, cyclohexyl, and optionally substituted phenyl, where the optional substituents are selected from the group consisting of: R²; OR⁵, where R⁵ is selected from H, CH₃, CH₂F, CHF₂, CF₃ and cyclopropyl; benzyloxy; halo; cyano; amino; C₅ heteroaryl, optionally substituted by methyl, CH₂OH, CH₂OCH₃ or ═O; phenyl; pyridyl, optionally substituted with methyl; COQ⁵, where Q⁵ is selected from OH and NR^{N1}R^{N2}; and CH₂OQ⁶, where Q⁶ is H or Me;
R¹ is selected from the group consisting of: F; phenyl; pyridyl; C₅ heteroaryl, optionally substituted by methyl, CH₂OCH₃, CH₂CF₃, CHF₂, NH₂, or ═O; C₉ heteroaryl; OH; OMe; OPh; COQ⁴, where Q⁴ is selected from OH, C₁₋₃ alkyloxy, NR^{N5}R^{N6}, where R^{N5} is selected from H and Me, and R^{N6} is selected from C₁₋₄ alkyl, which itself may be substituted by CONHMe, or where R^{N5} and R^{N6} together with the N atom to which they are bound form a C₄₋₆ N-containing heterocyclyl group, (CH₂)ₙ₁CONR^{N7}R^{N8}, where n1 is 1 to 3, and R^{N7} and R^{N8} are independently selected from H and Me, and O(CH₂)ₙ₂CONR^{N9}R^{N10}, where n2 is 1 or 3 And and R^{N9} and R^{N10} are independently selected from H and Me; (CH₂)ₙ OQ⁷, where n is 1 or 2 and Q⁷ is H or Me; NHCO₂Q⁸, where Q⁸ is C₁₋₃ alkyl; and OCONR^{N5}R^{N6};
R⁴ is selected from H, F and methyl; or
R¹ and R⁴ together with the carbon atom to which they are bound may form a C₄₋₆ cycloalkyl; and
when Cy is pyridyl, cyclohexyl or substituted phenyl, R¹ may additional be selected from H.

2. A compound according to claim 1, wherein X¹, X² and X³ are CH and X⁴ is Cy.

3. A compound according to claim 1, wherein:
(a) X¹ is N; or
(b) X² is N; or
(c) X³ is N; or
(d) X⁴ is N.

4. A compound according to claim 1, wherein Y is selected from the group consisting of:
(a) H;
(b) halo;
(c) I;
(d) F;
(e) cyano;
(f) CH₃;
(g) CH₂F;
(h) CHF₂;
(i) CF₃;
(j) ethynyl; and
(k) cyclopropyl.

5-16. (canceled)

17. A compound according to claim 1, wherein Y is OR³.

18-22. (canceled)

23. A compound according to claim 1, wherein Y is NR^{N1}R^{N2}, and
(a) R^{N1} and R^{N2} are both H;
(b) R^{N1} and R^{N2} are both Me; or
(c) R^{N1} is H and R^{N2} is Me.

24-26. (canceled)

27. A compound according to claim 1, wherein Y is COQ¹.

28-35. (canceled)

36. A compound according to claim 1, wherein Y is NHSO₂Q³.

37. (canceled)

38. A compound according to claim 1, wherein Y is pyridyl.

39. A compound according to claim 1, wherein Y is C₅ heteroaryl, which is optionally substituted, wherein the substituent group on the C₅ heteroaryl is selected from unsubstituted C₁₋₃ alkyl, C₁₋₃ alkyl substituted by OH, and C₁₋₃ alkyl substituted by CONR^{N1}R^{N2}.

40-44. (canceled)

45. A compound according to claim 1, wherein Y is SO₂Me.

46. A compound according to claim 1, wherein Y is C₁₋₃ alkyl, substituted by NHZ, where Z is H, Me, SO₂Me, or COMe, or Y is C₁₋₃ alkyl, substituted by OH.

47-52. (canceled)

53. A compound according to claim 1, wherein R¹ is selected from the group consisting of:
(a) H;
(b) F;
(c) phenyl;
(d) pyridyl;
(e) C₅ heteroaryl, optionally substituted by methyl, CH₂OCH₃, CH₂CF₃, CHF₂, NH₂, or ═O;
(f) C₉ heteroaryl;
(g) OH;
(h) OMe;
(i) OPh;
(j) COQ⁴;
(k) (CH₂)ₙOQ⁷;
(l) NHCO₂Q⁸, where Q⁸ is C₁₋₃ alkyl; and
(m) OCONR^{N5}R^{N6}.

54-88. (canceled)

88. A compound according to claim 1, wherein R⁴ is H.

89. (canceled)

90. (canceled)

91. A compound according to claim 1, wherein R¹ and R⁴ together with the carbon atom to which they are bound form a C₄₋₆ cycloalkyl.

92-94. (canceled)

95. A compound according to claim 1, wherein Cy is selected from the group consisting of:
(a) pyridyl;
(b) oxazolyl;
(c) cyclohexyl; and
(d) unsubstituted phenyl.

96-98. (canceled)

99. A compound according to claim 1, wherein Cy is phenyl bearing a single substituent.

100-102. (canceled)

103. A compound according to claim 99, wherein the phenyl substituent is selected from the group consisting of:
a) CH₃;
b) CH₂F;
c) CHF₂;
d) CF₃;
e) OCH₃;
f) OCH₂F;
g) OCHF₂;
h) OCF₃; and
i) O-cyclopropyl.

104. (canceled)

105. A compound according to claim 99, wherein the phenyl substituent is selected from the group consisting of:
(a) benzyloxy;
(b) halo;
(c) cyano;
(d) NH₂;
(e) C₅ heteroaryl, optionally substituted by methyl, CH₂OH, CH₂OCH₃ or ═O;
(f) phenyl;
(g) pyridyl, optionally substituted with methyl;
(h) CO₂H;
(i) CO₂Me;
(j) CONR^{N1}R^{N2}, wherein:
i) R^{N1} and R^{N2} are both H; or
ii) R^{N1} and R^{N2} are both Me; or
iii) R^{N1} is H and R^{N2} is Me;
(k) CH₂OH; and
(l) CH₂OMe.

106-124. (canceled)

125. A compound according to claim 1, wherein R¹ is H and Cy has a substituent in the 2-position, selected from OCHF₂ and a C₅ heteroaryl group selected from oxazolyl, pyrazolyl and triazolyl.

126. A compound according to claim 1, wherein R¹ is selected from oxazolyl, methyl-oxadiazolyl and pyrazolyl and Cy bears no substituent in the 2-position.

127-129. (canceled)

128. (canceled)

130. A pharmaceutical composition comprising a compound according to claim 1 and a pharmaceutically acceptable excipient.

131. A method of treatment of cancer, comprising administering to a patient in need of treatment, a compound according to claim 1.

132. A method according to claim 131, wherein the compound is administered simultaneously or sequentially with radiotherapy and/or chemotherapy

133-136. (canceled)
